(19) United States
(12) Patent Application Publication
(10) Pub. No.: US 2017/0037028 A1
(43) Pub. Date: Feb. 9, 2017

(54) GLUCOSE TRANSPORT INHIBITORS

(71) Applicant: BAYER PHARMA AKTIENGESELLSCHAFT, Berlin (DE)

(72) Inventors: Iring HEISLER, Wuppertal (DE); Thomas MÜLLER, Langenfeld (DE); Holger SIEBENEICHER, Berlin (DE); Bernd BUCHMANN, Hohen Neuendorf (DE); Arwed CLEVE, Berlin (DE); Judith GÜNTHER, Berlin (DE); Marcus KOPPITZ, Berlin (DE); Melanie HEROULT, Düsseldorf (DE); Roland NEUHAUS, Berlin (DE); Heike PETRUL, Berlin (DE); Maria QUANZ-SCHÖFFEL, Berlin (DE)

(21) Appl. No.: 15/106,814

(22) PCT Filed: Dec. 16, 2014
(86) PCT No.: PCT/EP2014/077879

§ 371 Date: Jun. 20, 2016

(30) Foreign Application Priority Data
Dec. 20, 2013 (EP) 13198787.7

(51) Int. Cl. C07D 401/12; A61K 31/473; C07D 401/14; A61K 31/5377; C07D 471/04; C07D 413/14; C07D 417/14; C07D 495/10; C07D 405/14; A61K 31/496; A61K 31/4709; C07D 409/14

(52) U.S. Cl. CPC C07D 401/12; A61K 31/4709; A61K 31/473; C07D 401/14; A61K 31/5377; C07D 409/14; C07D 413/14; C07D 417/14; C07D 495/10; C07D 405/14; A61K 31/496; C07D 471/04

(57) ABSTRACT

The present invention relates to chemical compounds that selectively inhibit glucose transporter 1 (GLUT1), to methods of preparing said compounds, to pharmaceutical compositions and combinations comprising said compounds, to the use of said compounds for manufacturing a pharmaceutical composition for the treatment or prophylaxis of a disease, as well as to intermediate compounds useful in the preparation of said compounds.

The present invention relates to chemical compounds that selectively inhibit glucose transporter 1 (GLUT1), to methods of preparing said compounds, to pharmaceutical compositions and combinations comprising said compounds, to the use of said compounds for manufacturing a pharmaceutical composition for the treatment or prophylaxis of a disease, as well as to intermediate compounds useful in the preparation of said compounds.

### BACKGROUND OF THE INVENTION

Glucose is an essential substrate for metabolism in most cells. Because glucose is a polar molecule, transport through biological membranes requires specific transport proteins. Transport of glucose through the apical membrane of intestinal and kidney epithelial cells depends on the presence of secondary active Na⁺/glucose symporters, SGLT-1 and SGLT-2, which concentrate glucose inside the cells, using the energy provided by co-transport of Na⁺ ions down their electrochemical gradient. Facilitated diffusion of glucose through the cellular membrane is otherwise catalyzed by glucose carriers (protein symbol GLUT, gene symbol SLC2 for Solute Carrier Family 2) that belong to a superfamily of transport facilitators (major facilitator superfamily) including organic anion and cation transporters, yeast hexose transporter, plant hexose/proton symporters, and bacterial sugar/proton symporters.

Basal glucose transporters (GLUTs) function as glucose channels and are required for maintaining the basic glucose needs of cells. These GLUTs are constitutively expressed and functional in cells and are not regulated by (or sensitive to) insulin. ALL cells use both glycolysis and oxidative phosphorylation in mitochondria but rely overwhelmingly on oxidative phosphorylation when oxygen is abundant, switching to glycolysis at times of oxygen deprivation (hypoxia), as it occurs in cancer. In glycolysis, glucose is converted to pyruvate and two ATP molecules are generated in the process. Cancer cells, because of their faster proliferation rates, are predominantly in a hypoxic (Low oxygen) state. Therefore, cancer cells use glycolysis (lactate formation) as their predominant glucose metabolism pathway. Such a glycolytic switch not only gives cancer higher potentials for metastasis and invasiveness, but also increases cancer's vulnerability to external interference in glycolysis. The reduction of basal glucose transport is likely to restrict glucose supply to cancer cells, leading to glucose deprivation that forces cancer cells to slow down growth or to starve.

All known GLUT proteins contain 12 transmembrane domains and transport glucose by facilitating diffusion, an energy-independent process. GLUT1 transports glucose into cells probably by alternating its conformation. According to this model, GLUT1 exposes a single substrate-binding site toward either the outside or the inside of the cell. Binding of glucose to one site triggers a conformational change, releasing glucose to the other side of the membrane. Results of transgenic and knockout animal studies support an important role for these transporters in the control of glucose utilization, glucose storage and glucose sensing. The GLUT proteins differ in their kinetics and are tailored to the needs of the cell types they serve. Although more than one GLUT protein may be expressed by a particular cell type, cancers frequently overexpress GLUT1, which is a high affinity glucose transporter, and its expression Level is correlated with invasiveness and metastasis potentials of cancers, indicating the importance of upregulation of glucose transport in cancer cell growth and in the severity of cancer malignancy. GLUT1 expression was also found to be significantly higher than that of any other glucose transporters.

Evidence indicates that cancer cells are more sensitive to glucose deprivation than normal cells. Numerous studies strongly suggest that basal glucose transport inhibition induces apoptosis and blocks cancer cell growth. Anti-angiogenesis has been shown to be a very effective way to restrict cancer growth and cause cancer ablation.

Reduced GLUT1 expression following transfection of GLUT1 antisense cDNA into cancer cell lines has been shown to suppress cell growth in vitro and tumor growth in vivo, and to reduce in vitro invasiveness of cells (Noguchi Y. et al. Cancer Lett 154(2), 2000, 175-182; Ito S. et al. J Natl Cancer Inst 94(14), 2002, 1080-1091).

It has been demonstrated that GLUT1 is the most highly expressed hexose transporter in ErbB2- and PyVMT-induced mouse mammary carcinoma models, and that reducing the Level of GLUT1 using shRNA or Cre/lox results in reduced glucose usage, reduced growth on plastic and in soft agar, and impaired tumor growth in nude mice (Christian D. Young et al., PLoS ONE, August 2011, Volume 6, Issue 8, e23205, 1-12).

Therefore, inhibition of GLUT1 represents a promising approach for the treatment of proliferative disorders including solid tumours such as carcinomas and sarcomas and Leukaemias and lymphoid malignancies or other disorders associated with uncontrolled cellular proliferation.

Different compounds have been disclosed in prior art which show an inhibitory effect on GLUT1. For example, WO2011/119866(A1) discloses composition and methods for glucose transport inhibition; WO2012/051117(A2) and WO2013/155338(A2) disclose substituted benzamides as GLUT1 inhibitors.

Compounds showing a certain structural similarity to the compounds of the present invention are disclosed in prior art. WO97/36881(A1) discloses arylheteroaryl-containing compounds which inhibit farnesyl-protein transferase. WO00/07996(A2) discloses pyrazole estrogen receptor agonist and antagonist compounds. WO01/21160(A2) discloses carboxamide derivatives as inhibitors of herpesviridae. WO03/037274(A2) and WO2004/099154(A2) disclose pyrazole-amides as inhibitors of sodium channels. WO2004/098528(A2) discloses pyrazole derived compounds as inhibitors of p38 kinase. WO2006/132197(A1) discloses heterocyclic compounds as inhibitors of 11 β-hydroxysteroid dehydrogenase type 1. WO2006/062249(A1) discloses compounds for the prevention, therapy or improvement of a disease to which the activation of a thrombopoietin receptor is effective. WO2008/126899(A1) discloses 5-membered heterocyclic compounds as inhibitors of xanthine oxidase. WO2008/008286(A2) discloses substituted pyrazoles as ghrelin receptor antagonists. WO2009/025793(A2) discloses compounds that function as bitter taste blockers. WO2009/027393(A2) and WO2010/034737(A1) disclose pyrazole compounds for controlling invertebrate pests. WO2009/099193(A1) discloses compounds having inhibitory action on melanin production. WO2009/119880(A1) discloses pyrazole derivatives having an androgen receptor antagonistic action. WO2011/050305(A1) and WO2011/050316(A1) disclose pyrazole compounds as allosteric modulators of mGluR⁴ receptor activity. WO2011/126903(A2) discloses multisubstituted aromatic compounds including substituted pyrazolyl as thrombin inhibitors. WO2004/110350(A2) discloses compounds modulating amyloid beta. WO2009/055917(A1) discloses inhibitors of histone deacetylase. WO02/23986(A1) discloses 4-acylaminopyrazole derivatives exhibiting fungicidal activities. WO03/051833(A2) discloses heteroaryl substituted pyrazole compounds as mGluR⁵ modulators. WO2009/076454(A2) discloses compounds which modulate the activity of store-operated calcium channels. WO99/32454(A1) discloses nitrogen containing heteroaromatics with ortho-substituted P1 groups as factor Xa inhibitors. WO2004/037248(A2) and WO2004/043951(A1) disclose compounds as modulators of the peroxisome proliferator activated receptors. WO 2013/109991(A1) discloses various heterocyclic compounds for the treatment of neurodegenerative diseases. WO 2014031936(A2) discloses heteroaromatic compounds as α7β1 Integrin modulators.

However, the state of the art described above does not specifically disclose the compounds of general formula (I) of the present invention, or a tautomer, a stereoisomer, an N-oxide, a hydrate, a solvate, or a salt thereof, or a mixture of same, as described and defined herein, and as hereinafter referred to as “compounds of the present invention”, or their pharmacological activity.

### SUMMARY of the INVENTION

The present invention covers compounds of general formula (I):

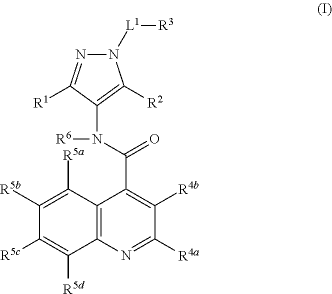

in which:
R¹ represents a C₁-C₃-alkyl-, halo-C₁-C₃-alkyl-, cyano-, —C(═O)O—R¹⁰ or —C(═O)N(R^{10a})R^{10b} group;
R² represents a C₁-C₃-alkyl-, halo-C₁-C₃-alkyl-, cyano-, —C(═O)O—R¹⁰ or —C(═O)N(R^{10a})R^{10b} group;
R³ represents a group selected from: aryl-, heteroaryl-, C₅-C₆-cycloalkyl-, and 5- to 6-membered heterocycloalkyl-;
   wherein said 5- to 6-membered heterocycloalkyl- group is optionally benzocondensed;
   wherein said aryl-, heteroaryl-, C₅-C₆-cycloalkyl-, and 5- to 6-membered heterocycloalkyl- group is optionally substituted, one or more times, identically or differently, with -(L²)ₚ-R⁷;
   and wherein two -(L²)ₚ-R⁷ groups, if being present ortho to each other on an aryl- or heteroaryl- group optionally form a bridge selected from: *—C₃-C₈-alkylene-*, *—O(CH₂)₂O—*, *—O(CH₂)O—*, *—O(CF₂)O—*, *—CH₂C(R^{10a})(R^{10b})O—*, *—C(═O)N(R^{10a})CH₂—*, *—N(R^{10a})C(═O)CH₂O—*, *—NHC(═O)NH—*; wherein each * represents the point of attachment to said aryl- or heteroaryl- group;
R^{4a} represents a hydrogen atom or a halogen atom or a group selected from: cyano-, hydroxy-, C₁-C₃-alkyl-, halo-C₁-C₃-alkyl-, C₁-C₃-alkoxy-, halo-C₁-C₃-alkoxy-, C₃—C-cycloalkyl-, 4- to 7-membered heterocycloalkyl-, —C(═O)—OR¹⁰, —C(═O)N(R^{10a})R^{10b}, —C(═O)—N(R^{10a})—S(═O)₂—R¹⁰, —SR¹⁰, —S(═O)—R¹⁰, —S(═NR¹¹)—R¹⁰, —S(═O)₂—R¹⁰, —S(═O)₂—N(R^{10a})R^{10b}, —S(═O)(═NR¹¹)—R¹⁰, —N(R^{10a})R^{10b};
R^{4b} represents a hydrogen atom or a group selected from: C₁-C₃-alkoxy-, C₁-C₃-alkyl-, cyano-;
or
R^{4a} and together R^{4b} form a —C₃-C₅-alkylene- group;
R^{5a}, R^{5b}, R^{5c}, R^{5d}
   independently from each other represent a hydrogen atom, a halogen atom or a group selected from:
   cyano-, —NO₂, C₁-C₃-alkyl-, halo-C₁-C₃-alkyl-, C₁-C₃-alkoxy-, halo-C₁-C₃-alkoxy-, phenyl-, heteroaryl-, —C(═O)R¹⁰, —C(═O)N(H)R¹⁰, —C(═O)N(R^{10a})R^{10b}, —C(═O)O—R¹⁰, —N(R^{10a})R^{10b}, —N(H)C(═O)R¹⁰, —N(R^{10a})C(═O)R^{10b}, —N(H)C(═O)N(R^{10a})R^{10b}, —N(R^{10a})C(═O)N(R^{10b})R^{10c}, —N(R^{10a})C(═O)C(═O)N(R^{10b})R^{10c}, —N(H)C(═O)OR¹⁰, —N(R^{10a})C(═O)OR^{10b}, —N(H)S(═O)₂R¹⁰, —N(R^{10a})S(═O)₂R^{10b}, —OR¹⁰, —O(C═O)R¹⁰, —O(C═O)N(R^{10a})R^{10b}, —O(C═O)OR¹⁰, —SR¹⁰, —S(═O)R¹⁰, —S(═O)₂R¹⁰, —S(═O)₂N(H)R¹⁰, —S(═O)₂N(R^{10a})R^{10b} or —S(═O)(═NR^{10a})R^{10b},
   said phenyl- or heteroaryl- group being optionally substituted one or more times, identically or differently, with a group selected from: halo-, cyano-, C₁-C₃-alkyl-, halo-C₁-C₃-alkyl-, C₁-C₃-alkoxy-;
R⁶ represents a hydrogen atom or group selected from: C₁-C₃-alkyl-, C₁-C₃-alkoxy-(L²)-, hydroxy-C₁-C₃-alkyl-, aryl-(L²)-, heteroaryl-(L²)-;
R⁷ represents a group selected from: oxo, C₁-C₆-alkyl-, C₃-C₇-cycloalkyl-, 4- to 7-membered heterocycloalkyl-, halo-C₁-C₄-alkyl-, hydroxy-C₁-C₄-alkyl-, cyano-C₁-C₄-alkyl-, C₂-C₄-alkenyl-, C₂-C₄-alkynyl-, C₁-C₄-alkoxy-, halo-C₁-C₄-alkoxy-, —OH, —CN, halo-, —C(═O)R⁸, —C(═O)—O—R⁸, —C(═O)N(R^{8a})R^{8b}, —N(R^{10a})R^{10b}, —S(═O)₂R⁸, —S(═O)(═NR¹¹)—R¹⁰, phenyl-, 5- to 6-membered heteroaryl-;
R⁸ represents a hydrogen atom or a C₁-C₆-alkyl-, halo-C₁-C₃-alkyl-, cyano-C₁-C₄-alkyl-, C₁-C₃-alkoxy-C₁-C₃-alkyl-, C₃-C₇-cycloalkyl-, phenyl-, 5- to 6-membered heteroaryl- or benzyl- group;
R^{8a}, R^{8b}
   represent, independently from each other, a hydrogen atom, or a C₁-C₁₀-alkyl-, C₃-C₇-cycloalkyl-, (C₃-C₇-cycloalkyl)-(L³)-, C₃-C₆-alkenyl-, C₃-C₆-alkynyl-, 4- to 10-membered heterocycloalkyl-,
   (4- to 10-membered heterocycloalkyl)-(L³)-, phenyl-, heteroaryl-, phenyl-(L³)-, (phenyl)-O-(L³)-, heteroaryl-(L³)-, or
   (aryl)-(4- to 10-membered heterocycloalkyl)- group;
   said C₁-C₁₀-alkyl-, C₃-C₇-cycloalkyl-, (C₃-C₇-cycloalkyl)-(L³)-, C₃-C₆-alkenyl-, C₃-C₆-alkynyl-, 4- to 10-membered heterocycloalkyl-, (4- to 10-membered heterocycloalkyl)-(L³)-, phenyl-, heteroaryl-, phenyl-(L³)-, (phenyl)-O-(L³)-, heteroaryl-(L³)-, and (aryl)-(4- to 10-membered heterocycloalkyl)- group being optionally substituted one or more times, identically or differently, with R⁹;
or
R^{8a} and R^{8b}, together with the nitrogen atom they are attached to,
   represent a 4- to 10-membered heterocycloalkyl-group, said 4- to 10-membered heterocycloalkyl- group being optionally substituted one or more times, identically or differently, with R⁹;
R⁹ represents a halogen atom, or a oxo, C₁-C₃-alkyl-, halo-C₁-C₃-alkyl-, hydroxy-C₁-C₃-alkyl-, —CN, —C(═O)R¹⁰, —C(═O)N(H)R¹⁰, —C(═O)N(R^{10a})R^{10b}, —C(═O)O—R¹⁰, —N(R^{10a})R^{10b}, —NO₂, —N(H)C(═O)R¹⁰, —N(R^{10a})C(═O)R^{10b}, —N(H)C(═O)N(R^{10a})R^{10b}, —N(R^{10a})C(═O)N(R^{10b})R^{10c}, —N(H)C(═O)OR¹⁰, —N(R^{10a})C(═O)OR^{10b}, —N(H)S(═O)₂R¹⁰, —N(R^{10a})S(═O)₂R^{10b}, —OR¹⁰, —O(C═O)R¹⁰, —O(C═O)N(R^{10a})R^{10b}, —O(C═O)OR¹⁰, —SR¹⁰, —S(═O)R¹⁰, —S(═O)₂R¹⁰, —S(═O)₂N(H)R¹⁰, —S(═O)₂N(R^{10a})R^{10b}, —S(═O)(═NR^{10a})R^{10b} or a tetrazolyl- group;
or
   two R⁹ groups present ortho to each other on a phenyl- or heteroaryl-ring form a bridge selected from: *—C₃-C₅-alkylene-*, *—O(CH₂)₂O—*, *—O(CH₂)O—*, *—O(CF₂)O—*, *—CH₂C(R^{10a})(R^{10b})O—*, *—C(═O)N(R^{10a})CH₂—*, *—N(R^{10a})C(═O)CH₂O—*, *—NHC(═O)NH—*; wherein each * represents the point of attachment to said phenyl- or heteroaryl- ring;
R¹⁰, R^{10a}, R^{10b}, R^{10c}
   represent, independently from each other, a hydrogen atom or a group selected from: C₁-C₃-alkyl-, halo-C₁-C₃-alkyl-, hydroxy-C₁-C₃-alkyl-, C₁-C₃-alkoxy-C₁-C₃-alkyl-, C₃-C₇-cycloalkyl-, said C₁-C₃-alkyl- group being optionally substituted once with —N(R¹²)R^{12a};
or
R^{10a} and R^{10b}, together with the nitrogen atom they are attached to,
   represent a 4- to 7-membered heterocycloalkyl- group, said 4- to 7-membered heterocycloalkyl-group being optionally substituted one or more times, identically or differently, with R¹³;
R¹¹ represents a hydrogen atom or a cyano-, C₁-C₃-alkyl-, —C(═O)R¹⁰, —C(═O)N(H)R¹⁰, —C(═O)N(R^{10a})R^{10b} or —C(═O)O—R¹⁰ group;
R¹², R^{12a}
   represent, independently from each other, a hydrogen atom or a C₁-C₃-alkyl- group,
   or,
R¹², R^{12a},
   together with the nitrogen atom they are attached to, represent a 4- to 7-membered heterocycloalkyl- group;
R¹³ represents a halogen atom or a cyano, hydroxy, oxo, C₁-C₃-alkyl-, trifluoromethyl-, —C(═O)R¹⁰ or —C(═O)O—R¹⁰ group;
L¹ represents a group selected from: —C₁-C₄-alkylene-, —CH₂—CH═CH—, —C(phenyl)(H)—, —CH₂—CH₂—O—, —CH₂—C(═O)—N(H)—, —CH₂—C(═O)—N(R^{10a})—;
L² represents a group selected from: —CH₂—, —CH₂—CH₂—, —CH₂—CH₂—CH₂—;
L³ represents a —C₁-C₆-alkylene- group;
p is an integer of 0 or 1;
   or a tautomer, a stereoisomer, an N-oxide, a hydrate, a solvate, or a salt thereof, or a mixture of same.

The present invention further relates to methods of preparing compounds of general formula (I), to pharmaceutical compositions and combinations comprising said compounds, to the use of said compounds for manufacturing a pharmaceutical composition for the treatment or prophylaxis of a disease, as well as to intermediate compounds useful in the preparation of said compounds.

### DETAILED DESCRIPTION OF THE INVENTION

The terms as mentioned in the present text have preferably the following meanings:

The term “halogen atom” or “halo-” is to be understood as meaning a fluorine, chlorine, bromine or iodine atom.

The term “oxo” is to be understood as preferably meaning an oxygen atom attached to an atom featuring suitable bonding valence, such as a saturated carbon atom or a sulfur atom, by a double bond, resulting in the formation e.g. of a carbonyl group —C(═O)— or a sulfonyl group —S(═O)₂—.

The term “C₁-C₁₀-alkyl-” is to be understood as preferably meaning a linear or branched, saturated, monovalent hydrocarbon group having 1, 2, 3, 4, 5, 6, 7, 8, 9 or 10 carbon atoms, e.g. a methyl-, ethyl-, propyl-, butyl-, pentyl-, hexyl-, iso-propyl-, iso-butyl-, sec-butyl-, tert-butyl-, iso-pentyl-, 2-methylbutyl-, 1-methylbutyl-, 1-ethylpropyl-, 1,2-dimethylpropyl-, neo-pentyl-, 1,1-dimethylpropyl-, 4-methylpentyl-, 3-methylpentyl-, 2-methylpentyl-, 1-methylpentyl-, 2-ethylbutyl-, 1-ethylbutyl-, 3,3-dimethylbutyl-, 2,2-dimethylbutyl-, 1,1-dimethylbutyl-, 2,3-dimethylbutyl-, 1,3-dimethylbutyl-, or 1,2-dimethylbutyl-, heptyl-, octyl-, nonyl- or decyl- group, or an isomer thereof. Particularly, said group has 1, 2, 3, 4, 5 or 6 carbon atoms (“C₁-C₆-alkyl-”), more particularly 1, 2, 3 or 4 carbon atoms (“C₁-C₄-alkyl-”), e.g. a methyl-, ethyl-, propyl-, butyl-, iso-propyl-, iso-butyl-, sec-butyl-, tert-butyl-group, even more particularly 1, 2 or 3 carbon atoms (“C₁-C₃-alkyl-”), e.g. a methyl-, ethyl-, n-propyl- or iso-propyl- group.

The term “—C₁-C₈-alkylene-” is understood as preferably meaning a linear or branched, saturated, divalent hydrocarbon chain (or “tether”) having 1, 2, 3, 4, 5, 6, 7 or 8 carbon atoms, e.g. —CH₂— (“methylene” or “—C₁-alkylene-”) or, for example —CH₂—CH₂— (“ethylene” or “—C₂-alkylene-”), —CH₂—CH₂—CH₂—, —C(H)(CH₃)—CH₂— or —C(CH₃)₂—) (“propylene” or “—C₃-alkylene-”), or, for example —CH₂—C(H)(CH₃)—CH₂—, —CH₂—C(CH₃)₂—), —CH₂—CH₂—CH₂—CH₂— (“butylene” or “—C₄-alkylene-”), “—C₅-alkylene-”, e.g. —CH₂—CH₂—CH₂—CH₂—CH₂— (“n-pentylene”), or “—C₆-alkylene-”, e.g. —CH₂—CH₂—CH₂—CH₂—CH₂—CH₂— (“n-hexylene”) group. Particularly, said alkylene tether has 1, 2, 3, 4, or 5 carbon atoms (“—C₁-C₅-alkylene-”), more particularly 1 or 2 carbon atoms (“—C₁-C₂-alkylene-”), or, 3, 4, or 5 carbon atoms (“—C₃-C₅-alkylene-”).

The term “halo-C₁-C₄-alkyl-” is to be understood as preferably meaning a linear or branched, saturated, monovalent hydrocarbon group in which the term “C₁-C₄-alkyl-” is defined supra, and in which one or more of the hydrogen atoms is replaced, identically or differently, by a halogen atom. Preferred are halo-C₁-C₃-alkyl- groups. Particularly, said halogen atom is F, resulting in a group also referred to as “fluoro-C₁-C₃-alkyl-”. Said halo-C₁-C₃-alkyl- group or fluoro-C₁-C₃-alkyl- group is, for example, —CF₃, —CHF₂, —CH₂F, —CF₂CF₃, or —CH₂CF₃.

The term “cyano-C₁-C₄-alkyl-” is to be understood as preferably meaning a linear or branched, saturated, monovalent hydrocarbon group in which the term “C₁-C₄-alkyl-” is defined supra, and in which one or more of the hydrogen atoms is replaced by a cyano group. Said cyano-C₁-C₄-alkyl- group is, for example, —CH₂CN, —CH₂CH₂—CN, —C(CN)H—CH₃, —C(CN)H—CH₂CN, or —CH₂CH₂CH₂CH₂—CN.

The term “hydroxy-C₁-C₄-alkyl-” is to be understood as preferably meaning a linear or branched, saturated, monovalent hydrocarbon group in which the term “C₁-C₄-alkyl-” is defined supra, and in which one or more of the hydrogen atoms is replaced by a hydroxy group with the proviso that not more than one hydrogen atom attached to a single carbon atom is being replaced. Preferred are hydroxy-C₁-C₃-alkyl- groups. Said hydroxy-C₁-C₄-alkyl- group, or, preferably, hydroxy-C₁-C₃-alkyl- group is, for example, —CH₂OH, —CH₂CH₂—OH, —C(OH)H—CH₃, or —C(OH)H—CH₂OH.

The term “C₁-C₄-alkoxy-” is to be understood as preferably meaning a linear or branched, saturated, monovalent group of formula —O—(C₁-C₄-alkyl-), in which the term “C₁-C₄-alkyl-” is defined supra, e.g. a methoxy-, ethoxy-, n-propoxy-, iso-propoxy-, n-butoxy-, tert-butoxy. Preferred are C₁-C₃-alkoxy-groups.

The term “halo-C₁-C₄-alkoxy-” is to be understood as preferably meaning a linear or branched, saturated, monovalent C₁-C₄-alkoxy- group, as defined supra, in which one or more of the hydrogen atoms is replaced, identically or differently, by a halogen atom. Preferred are halo-C₁-C₃-alkoxy-groups. Particularly, said halogen atom is F, resulting in a group also referred to as “fluoro-C₁-C₄-alkoxy-”, or, preferably “fluoro-C₁-C₃-alkoxy-”. Said halo-C₁-C₄-alkoxy- group or fluoro-C₁-C₄-alkoxy- group is, for example, —OCF₃, —OCHF₂, —OCH₂F, —OCF₂CF₃, or —OCH₂CF₃.

The term “C₁-C₃-alkoxy-C₁-C₃-alkyl-” is to be understood as preferably meaning a linear or branched, saturated, monovalent C₁-C₃-alkyl- group, as defined supra, in which one or more of the hydrogen atoms is replaced, identically or differently, by a C₁-C₃-alkoxy group, as defined supra, e.g. methoxyalkyl-, ethoxyalkyl-, propyloxyalkyl- or iso-propoxyalkyl-.

The term “halo-C₁-C₃-alkoxy-C₁-C₃-alkyl-” is to be understood as preferably meaning a linear or branched, saturated, monovalent C₁-C₃-alkoxy-C₁-C₃-alkyl-group, as defined supra, in which one or more of the hydrogen atoms is replaced, in identically or differently, by a halogen atom. Particularly, said halogen atom is F, resulting in a group also referred to as “fluoro-C₁-C₃-alkoxy-C₁-C₃-alkyl-”. Said halo-C₁-C₃-alkoxy-C₁-C₃-alkyl- group or fluoro-C₁-C₃-alkoxy-C₁-C₃-alkyl- group is, for example, —CH₂CH₂OCF₃, —CH₂CH₂OCHF₂, —CH₂CH₂OCH₂F, —CH₂CH₂OCF₂CF₃, or —CH₂CH₂OCH₂CF₃.

The term “C₂-C₆-alkenyl-” is to be understood as preferably meaning a linear or branched, monovalent hydrocarbon group, which contains one or more double bonds, and which has 2, 3, 4, 5 or 6 carbon atoms, particularly 3, 4, 5 or 6 carbon atoms (“C₃-C₆-alkenyl-”), more particularly 2 or 4 carbon atoms (“C₂-C₄-alkenyl-”), or 3 or 4 carbon atoms (“C₃-C₄-alkenyl-”), it being understood that in the case in which said alkenyl- group contains more than one double bond, then said double bonds may be isolated from, or conjugated with, each other. Said alkenyl- group is, for example, a vinyl-, allyl-, (E)-2-methylvinyl-, (Z)-2-methylvinyl-, homoallyl-, (E)-but-2-enyl-, (Z)-but-2-enyl-, (E)-but-1-enyl-, (Z)-but-1-enyl-, pent-4-enyl-, (E)-pent-3-enyl-, (Z)-pent-3-enyl-, (E)-pent-2-enyl-, (Z)-pent-2-enyl-, (E)-pent-1-enyl-, (Z)-pent-1-enyl-, hex-5-enyl-, (E)-hex-4-enyl-, (Z)-hex-4-enyl-, (E)-hex-3-enyl-, (Z)-hex-3-enyl-, (E)-hex-2-enyl-, (Z)-hex-2-enyl-, (E)-hex-1-enyl-, (Z)-hex-1-enyl-, iso-propenyl-, 2-methylprop-2-enyl-, 1-methylprop-2-enyl-, 2-methylprop-1-enyl-, (E)-1-methylprop-1-enyl-, (Z)-1-methylprop-1-enyl-, 3-methylbut-3-enyl-, 2-methylbut-3-enyl-, 1-methylbut-3-enyl-, 3-methylbut-2-enyl-, (E)-2-methylbut-2-enyl-, (Z)-2-methylbut-2-enyl-, (E)-1-methylbut-2-enyl-, (Z)-1-methylbut-2-enyl-, (E)-3-methylbut-1-enyl-, (Z)-3-methylbut-1-enyl-, (E)-2-methylbut-1-enyl-, (Z)-2-methylbut-1-enyl-, (E)-1-methylbut-1-enyl-, (Z)-1-methylbut-1-enyl-, 1,1-dimethylprop-2-enyl-, 1-ethylprop-1-enyl-, 1-propylvinyl-, 1-isopropylvinyl-, 4-methylpent-4-enyl-, 3-methylpent-4-enyl-, 2-methylpent-4-enyl-, 1-methylpent-4-enyl-, 4-methylpent-3-enyl-, (E)-3-methylpent-3-enyl-, (Z)-3-methylpent-3-enyl-, (E)-2-methylpent-3-enyl-, (Z)-2-methylpent-3-enyl-, (E)-1-methylpent-3-enyl-, (Z)-1-methylpent-3-enyl-, (E)-4-methylpent-2-enyl-, (Z)-4-methylpent-2-enyl-, (E)-3-methylpent-2-enyl-, (Z)-3-methylpent-2-enyl-, (E)-2-methylpent-2-enyl-, (Z)-2-methylpent-2-enyl-, (E)-1-methylpent-2-enyl-, (Z)-1-methylpent-2-enyl-, (E)-4-methylpent-1-enyl-, (Z)-4-methylpent-1-enyl-, (E)-3-methylpent-1-enyl-, (Z)-3-methylpent-1-enyl-, (E)-2-methylpent-1-enyl-, (Z)-2-methylpent-1-enyl-, (E)-1-methylpent-1-enyl-, (Z)-1-methylpent-1-enyl-, 3-ethylbut-3-enyl-, 2-ethylbut-3-enyl-, 1-ethylbut-3-enyl-, (E)-3-ethylbut-2-enyl-, (Z)-3-ethylbut-2-enyl-, (E)-2-ethylbut-2-enyl-, (Z)-2-ethylbut-2-enyl-, (E)-1-ethylbut-2-enyl-, (Z)-1-ethylbut-2-enyl-, (E)-3-ethylbut-1-enyl-, (Z)-3-ethylbut-1-enyl-, 2-ethylbut-1-enyl-, (E)-1-ethylbut-1-enyl-, (Z)-1-ethylbut-1-enyl-, 2-propylprop-2-enyl-, 1-propylprop-2-enyl-, 2-isopropylprop-2-enyl-, 1-isopropylprop-2-enyl-, (E)-2-propylprop-1-enyl-, (Z)-2-propylprop-1-enyl-, (E)-1-propylprop-1-enyl-, (Z)-1-propylprop-1-enyl-, (E)-2-isopropylprop-1-enyl-, (Z)-2-isopropylprop-1-enyl-, (E)-1-isopropylprop-1-enyl-, (Z)-1-isopropylprop-1-enyl-, (E)-3,3-dimethylprop-1-enyl-, (Z)-3,3-dimethylprop-1-enyl-, 1-(1,1-dimethylethyl)ethenyl-, buta-1,3-dienyl-, penta-1,4-dienyl-, hexa-1,5-dienyl-, or methylhexadienyl- group. Particularly, said group is vinyl- or allyl-.

The term “C₂-C₆-alkynyl-” is to be understood as preferably meaning a linear or branched, monovalent hydrocarbon group which contains one or more triple bonds, and which contains 2, 3, 4, 5 or 6 carbon atoms, particularly 3, 4, 5 or 6 carbon atoms (“C₃-C₆-alkynyl-”), more particularly 2 or 4 carbon atoms (“C₂-C₄-alkynyl-”), or 3 or 4 carbon atoms (“C₃-C₄-alkynyl-”). Said C₂-C₆-alkynyl-group is, for example, ethynyl-, prop-1-ynyl-, prop-2-ynyl-, but-1-ynyl-, but-2-ynyl-, but-3-ynyl-, pent-1-ynyl-, pent-2-ynyl-, pent-3-ynyl-, pent-4-ynyl-, hex-1-ynyl-, hex-2-ynyl-, hex-3-ynyl-, hex-4-ynyl-, hex-5-ynyl-, 1-methylprop-2-ynyl-, 2-methylbut-3-ynyl-, 1-methylbut-3-ynyl-, 1-methylbut-2-ynyl-, 3-methylbut-1-ynyl-, 1-ethylprop-2-ynyl-, 3-methylpent-4-ynyl-, 2-methylpent-4-ynyl-, 1-methylpent-4-ynyl-, 2-methylpent-3-ynyl-, 1-methylpent-3-ynyl-, 4-methylpent-2-ynyl-, 1-methyl-pent-2-ynyl-, 4-methylpent-1-ynyl-, 3-methylpent-1-ynyl-, 2-ethylbut-3-ynyl-, 1-ethylbut-3-ynyl-, 1-ethylbut-2-ynyl-, 1-propylprop-2-ynyl-, 1-isopropylprop-2-ynyl-, 2,2-dimethylbut-3-ynyl-, 1,1-dimethylbut-3-ynyl-, 1,1-dimethylbut-2-ynyl-, or 3,3-dimethylbut-1-ynyl- group. Particularly, said alkynyl- group is ethynyl-, prop-1-ynyl-, or prop-2-ynyl-.

The term “C₃-C₇-cycloalkyl-” is to be understood as meaning a saturated, monovalent, monocyclic hydrocarbon ring which contains 3, 4, 5, 6 or 7 carbon atoms. Said C₃-C₇-cycloalkyl- group is for example a cyclopropyl-, cyclobutyl-, cyclopentyl-, cyclohexyl- or cycloheptyl- ring. Particularly, said ring contains 3, 4, 5 or 6 carbon atoms (“C₃-C₆-cycloalkyl-”), more particularly, said ring contains 5 or 6 carbon atoms (“C₅-C₆-cycloalkyl-”).

The term “4- to 10-membered heterocycloalkyl-” is to be understood as meaning a saturated, monovalent, mono- or bicyclic hydrocarbon ring which contains 3, 4, 5, 6, 7, 8 or 9 carbon atoms, and one or more heteroatom-containing groups selected from —O—, —S—, —S(═O)—, —S(═O)₂—, —NR^{a}, in which R^{a} represents a hydrogen atom or a C₁-C₆-alkyl- or C₃-C₇-cycloalkyl-group; it being possible for said heterocycloalkyl- group to be attached to the rest of the molecule via any one of the carbon atoms or, if present, a nitrogen atom. Heterospirocycloalkyl-, heterobicycloalkyl- and bridged heterocycloalkyl-, as defined infra, are also included within the scope of this definition.

The term “heterospirocycloalkyl-” is to be understood as meaning a saturated, monovalent bicyclic hydrocarbon radical in which the two rings share one common ring carbon atom, and wherein said bicyclic hydrocarbon radical contains 3, 4, 5, 6, 7, 8 or 9 carbon atoms, and one or more heteroatom-containing groups selected from —O—, —S—, —S(═O)—, —S(═O)₂—, —NR^{a}, in which R^{a} represents a hydrogen atom or a C₁-C₆-alkyl- or C₃-C₇-cycloalkyl-group; it being possible for said heterospirocycloalkyl- group to be attached to the rest of the molecule via any one of the carbon atoms or, if present, a nitrogen atom. Said heterospirocycloalkyl- group is, for example, azaspiro[2.3]hexyl-, azaspiro[3.3]heptyl-, oxaazaspiro[3.3]heptyl-, thiaazaspiro[3.3]heptyl-, oxaspiro[3.3]heptyl-, oxazaspiro[5.3]nonyl-, oxazaspiro[4.3]octyl-, oxazaspiro[5.5]undecyl-, diazaspiro[3.3]heptyl-, thiazaspiro[3.3]heptyl-, thiazaspiro[4.3]octyl-, or azaspiro[5.5]decyl-.

The term “heterobicycloalkyl-” is to be understood as meaning a saturated, monovalent bicyclic hydrocarbon radical in which the two rings share two immediately adjacent ring atoms, and wherein said bicyclic hydrocarbon radical contains 3, 4, 5, 6, 7, 8 or 9 carbon atoms, and one or more heteroatom-containing groups selected from —O—, —S—, —S(═O)—, —S(═O)₂—, —NR^{a}—, in which R^{a} represents a hydrogen atom or a C₁-C₆-alkyl- or C₃-C₇-cycloalkyl-group; it being possible for said heterobicycloalkyl- group to be attached to the rest of the molecule via any one of the carbon atoms or, if present, a nitrogen atom. Said heterobicycoalkyl- group is, for example, azabicyclo[3.3.0]octyl-, azabicyclo[4.3.0]nonyl-, diazabicyclo[4.3.0]nonyl-, oxazabicyclo[4.3.0]nonyl-, thiazabicyclo[4.3.0]nonyl-, or azabicyclo[4.4.0]decyl-.

The term “bridged heterocycloalkyl-” is to be understood as meaning a saturated, monovalent bicyclic hydrocarbon radical in which the two rings share two common ring atoms which are not immediately adjacent, and wherein said bicyclic hydrocarbon radical contains 3, 4, 5, 6, 7, 8 or 9 carbon atoms, and one or more heteroatom-containing groups selected from —O—, —S—, —S(═O)—, —S(═O)₂—, —NR^{a}, in which R^{a} represents a hydrogen atom, or a C₁-C₆-alkyl- or C₃-C₇-cycloalkyl- group; it being possible for said bridged heterocycloalkyl- group to be attached to the rest of the molecule via any one of the carbon atoms or, if present, a nitrogen atom. Said bridged heterocycloalkyl- group is, for example, azabicyclo[2.2.1]heptyl-, oxazabicyclo[2.2.1]heptyl-, thiazabicyclo[2.2.1]heptyl-, diazabicyclo[2.2.1]heptyl-, azabicyclo[2.2.2]octyl-, diazabicyclo[2.2.2]octyl-, oxazabicyclo[2.2.2]octyl-, thiazabicyclo[2.2.2]octyl-, azabicyclo[3.2.1]octyl-, diazabicyclo[3.2.1]octyl-, oxazabicyclo[3.2.1]octyl-, thiazabicyclo[3.2.1]octyl-, azabicyclo[3.3.1]nonyl-, diazabicyclo[3.3.1]nonyl-, oxazabicyclo[3.3.1]nonyl-, thiazabicyclo[3.3.1]nonyl-, azabicyclo[4.2.]nonyl-, diazabicyclo[4.2.]nonyl-, oxazabicyclo[4.2.1]nonyt, thiazabicyclo[4.2.1]nonyl-, azabicyclo[3.3.2]decyl-, diazabicyclo[3.3.2]decyl-, oxazabicyclo[3.3.2]decyl-, thiazabicyclo[3.3.2]decyl-, or azabicyclo[4.2.2]decyl-.

Particularly, said 4- to 10-membered heterocycloalkyl- can contain 3, 4, 5 or 6 carbon atoms, and one or more of the above-mentioned heteroatom-containing groups (a “4- to 7-membered heterocycloalkyl-”), more particularly said heterocycloalkyl- can contain 4 or 5 carbon atoms, and one or more of the above-mentioned heteroatom-containing groups (a “5- to 6-membered heterocycloalkyl-”).

Particularly, without being limited thereto, said heterocycloalkyl- can be a 4-membered ring, such as an azetidinyl-, oxetanyl-, or a 5-membered ring, such as tetrahydrofuranyl-, pyrrolidinyl-, imidazolidinyl-, pyrazolidinyl-, or a 6-membered ring, such as tetrahydropyranyl-, piperidinyl-, morpholinyl-, dithianyl-, thiomorpholinyl-, piperazinyl-, or trithianyl-, or a 7-membered ring, such as a diazepanyl- ring, for example.

The term “aryl-” is to be understood as preferably meaning a monovalent, aromatic, mono-, or bi- or tricyclic hydrocarbon ring system having 6, 7, 8, 9, 10, 11, 12, 13 or 14 carbon atoms (a “C₆-C₁₄-aryl-” group), particularly a group having 6 carbon atoms (a “C₆-aryl-” group), e.g. a phenyl- group; or a group having 9 carbon atoms (a “C₉-aryl-” group), e.g. an indanyl- or indenyl- group, or a group having 10 carbon atoms (a “C₁₀-aryl-” group), e.g. a tetralinyl-, dihydronaphthyl-, or naphthyl- group, or a biphenyl- group (a “C₁₂-aryl-” group), or a group having 13 carbon atoms, (a “C₁₃-aryl-” group), e.g. a fluorenyl- group, or a group having 14 carbon atoms, (a “C₁₄-aryl-” group), e.g. an anthracenyl- group. Preferably, the aryl- group is a phenyl- group.

The term “heteroaryl-” is understood as preferably meaning an “aryl-” group as defined supra, in which at least one of the carbon ring atoms is replaced by a heteroatom selected from oxygen, nitrogen, and sulphur. The “heteroaryl-” group contains 5, 6, 7, 8, 9, 10, 11, 12, 13 or 14 ring atoms (a “5- to 14-membered heteroaryl-” group), particularly 5 or 6 or 9 or 10 ring atoms (a “5- to 10-membered heteroaryl-” group), more particularly 5 or 6 ring atoms (a “5- to 6-membered heteroaryl-” group). Particularly, heteroaryl- is selected from thienyl-, furanyl-, pyrrolyl-, oxazolyl-, thiazolyl-, imidazolyl-, pyrazolyl-, isoxazolyl-, isothiazolyl-, oxadiazolyl-, triazolyl-, thiadiazolyl-, thia-4H-pyrazolyl- etc., and benzo derivatives thereof, such as, for example, benzofuranyl-, benzothienyl-, benzoxazolyl-, benzisoxazolyl-, benzimidazolyl-, benzotriazolyl-, benzothiadiazolyl-, indazolyl-, indolyl-, isoindolyl-, etc.; or pyridyl-, pyridazinyl-, pyrimidyl-, pyrazinyl-, triazinyl-, etc., and benzo derivatives thereof, such as, for example, quinolinyl-, quinazolinyl-, isoquinolinyl-, etc.; or azocinyl-, indolizinyl-, purinyl-, etc., and benzo derivatives thereof; or cinnolinyl-, phthalazinyl-, quinazolinyl-, quinoxalinyl-, naphthpyridinyl-, pteridinyl-, carbazolyl-, acridinyl-, phenazinyl-, phenothiazinyl-, phenoxazinyl-, xanthenyl-, or oxepinyl-, and further bi- or tricyclic heteroaryl- groups featuring heteroatoms in more than one rings such as pyrrolopyrazolyl-, imidazopyrazolyl-, thienopyrrolyl-, pyrrolooxazolyl-, pyrrolopyridyl-, thienopyrimidyl-, imidazopyrimidyl-, imidazopyridazinyl-, imidazopyridyl-, thiazolopyridyl-, pyrazolopyridyl-, pyrrolotriazinyl-, etc.

In general, and unless otherwise mentioned, the heteroarylic or heteroarylenic radicals include all the possible isomeric forms thereof, e.g. the positional isomers thereof. Thus, for some illustrative non-restricting example, the term pyridyl- includes pyridin-2-yl-, pyridin-3-yl-, and pyridin-4-yl-; or the term thienyl- includes thien-2-yl- and thien-3-yl-. Preferably, the heteroaryl- group is a pyridyl- group.

The term “C₁-C₆”, as used throughout this text, e.g. in the context of the definition of “C₁-C₆-alkyl-” is to be understood as meaning an alkyl- group having a finite number of carbon atoms of 1 to 6, i.e. 1, 2, 3, 4, 5, or 6 carbon atoms. It is to be understood further that said term “C₁-C₆” is to be interpreted as any sub-range comprised therein, e.g. C₁-C₆, C₂-C₅, C₃-C₄, C₁-C₂, C₁-C₃, C₁-C₄, C₁-C₅, C₁-C₆; particularly C₁-C₂, C₁-C₃, C₁-C₄, C₁-C₅, C₁-C₆; more particularly C₁-C₄; in the case of “C₁-C₃-haloalkyl-” or “halo-C₁-C₃-alkoxy-” even more particularly C₁-C₂.

Similarly, as used herein, the term “C₂-C₆”, as used throughout this text, e.g. in the context of the definitions of “C₂-C₆-alkenyl-” and “C₂-C₆-alkynyl-”, is to be understood as meaning an alkenyl- group or an alkynyl group having a finite number of carbon atoms of 2 to 6, i.e. 2, 3, 4, 5, or 6 carbon atoms. It is to be understood further that said term “C₂-C₆” is to be interpreted as any sub-range comprised therein, e.g. C₂-C₆, C₃-C₅, C₃-C₄, C₂-C₃, C₂-C₄, C₂-C₅; particularly C₂-C₃.

Further, as used herein, the term “C₃-C₇”, as used throughout this text, e.g. in the context of the definition of “C₃-C₇-cycloalkyl”, is to be understood as meaning a cycloalkyl group having a finite number of carbon atoms of 3 to 7, i.e. 3, 4, 5, 6 or 7 carbon atoms. It is to be understood further that said term “C₃-C₇” is to be interpreted as any sub-range comprised therein, e.g. C₃-C₆, C₄-C₅, C₃-C₅, C₃-C₄, C₄-C₆, C₅-C₇; particularly C₃-C₆.

As used herein, the term “leaving group” refers to an atom or a group of atoms that is displaced in a chemical reaction as stable species taking with it the bonding electrons. The leaving group as used herein is suitable for nucleophilic aliphatic and/or aromatic substitution, e.g. a halogen atom, in particular chloro-, bromo- or iodo-, or a group selected from methanesulfonyloxy-, p-toluenesulfonyloxy-, trifluoromethanesulfonyloxy-, nonafluorobutanesulfonyloxy-, (4-bromo-benzene)sulfonyloxy-, (4-nitro-benzene)sulfonyloxy-, (2-nitro-benzene)-sulfonyloxy-, (4-isopropyl-benzene)sulfonyloxy-, (2,4,6-tri-isopropyl-benzene)-sulfonyloxy-, (2,4,6-trimethyl-benzene)sulfonyloxy-, (4-tert-butyl-benzene)sulfonyloxy-, benzenesulfonyloxy-, and (4-methoxy-benzene)sulfonyloxy-.

As used herein, the term “protective group” is a protective group attached to a nitrogen in intermediates used for the preparation of compounds of the general formula (I). Such groups are introduced e.g. by chemical modification of the respective amino group in order to obtain chemoselectivity in a subsequent chemical reaction. Protective groups for amino groups are descibed for example in T. W. Greene and P. G. M. Wuts in Protective Groups in Organic Synthesis, 3^{rd} edition, Wiley 1999; more specifically, said groups can be selected from substituted sulfonyl groups, such as mesyl-, tosyl- or phenylsulfonyl-, acyl groups such as benzoyl-, acetyl- or tetrahydropyranoyl-, or carbamate based groups, such as tert.-butoxycarbonyl- (Boc), or can include silicon, as in e.g. 2-(trimethylsilyl)ethoxymethyl- (SEM).

As used herein, the term “one or more times”, e.g. in the definition of the substituents of the compounds of the general formulae of the present invention, is understood as meaning “one, two, three, four or five times, particularly one, two, three or four times, more particularly one, two or three times, even more particularly one or two times”.

Where the plural form of the word compounds, salts, polymorphs, hydrates, solvates and the like, is used herein, this is taken to mean also a single compound, salt, polymorph, isomer, hydrate, solvate or the like.

The compounds of this invention contain one or more asymmetric centres, depending upon the location and nature of the various substituents desired. Asymmetric carbon atoms may be present in the (R) or (S) configuration. In certain instances, asymmetry may also be present due to restricted rotation about a given bond, for example, the central bond adjoining two substituted aromatic rings of the specified compounds.

Substituents on a ring may also be present in either cis or trans form. It is intended that all such configurations are included within the scope of the present invention.

Preferred compounds are those which produce the more desirable biological activity. Separated, pure or partially purified isomers and stereoisomers or racemic or diastereomeric mixtures of the compounds of this invention are also included within the scope of the present invention. The purification and the separation of such materials can be accomplished by standard techniques known in the art.

The optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallisation. The optically active bases or acids are then iberated from the separated diastereomeric salts. A different process for separation of optical isomers involves the use of chiral chromatography (e.g., chiral HPLC columns), with or without conventional derivatisation, optimally chosen to maximise the separation of the enantiomers. Suitable chiral HPLC columns are manufactured by Diacel, e.g., Chiracel OD and Chiracel OJ among many others, all routinely selectable. Enzymatic separations, with or without derivatisation, are also useful. The optically active compounds of this invention can likewise be obtained by chiral syntheses utilizing optically active starting materials.

In order to limit different types of isomers from each other reference is made to IUPAC Rules Section E (Pure Appt Chem 45, 11-30, 1976).

The invention also includes all suitable isotopic variations of a compound of the invention. An isotopic variation of a compound of the invention is defined as one in which at least one atom is replaced by an atom having the same atomic number but an atomic mass different from the atomic mass usually or predominantly found in nature. Examples of isotopes that can be incorporated into a compound of the invention include isotopes of hydrogen, carbon, nitrogen, oxygen, phosphorus, sulphur, fluorine, chlorine, bromine and iodine, such as ²H (deuterium), ³H (tritium), ¹¹C, ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I, respectively. Certain isotopic variations of a compound of the invention, for example, those in which one or more radioactive isotopes such as ³H or ¹⁴C are incorporated, are useful in drug and/or substrate tissue distribution studies. Tritiated and carbon-14, i.e., ¹⁴C, isotopes are particularly preferred for their ease of preparation and detectability. Further, substitution with isotopes such as deuterium may afford certain therapeutic advantages resulting from greater metabolic stability, for example, increased in vivo half-life or reduced dosage requirements and hence may be preferred in some circumstances. Isotopic variations of a compound of the invention can generally be prepared by conventional procedures known by a person skilled in the art such as by the illustrative methods or by the preparations described in the examples hereafter using appropriate isotopic variations of suitable reagents.

The present invention includes all possible stereoisomers of the compounds of the present invention as single stereoisomers, or as any mixture of said stereoisomers, in any ratio. Isolation of a single stereoisomer, e.g. a single enantiomer or a single diastereomer, of a compound of the present invention may be achieved by any suitable state of the art method, such as chromatography, especially chiral chromatography, for example.

Further, the compounds of the present invention may exist as tautomers. For example, any compound of the present invention which contains a pyrazole moiety as a heteroaryl group for example can exist as a 1H tautomer, or a 2H tautomer, or even a mixture in any amount of the two tautomers, or a triazole moiety for example can exist as a 1H tautomer, a 2H tautomer, or a 4H tautomer, or even a mixture in any amount of said 1H, 2H and 4H tautomers, viz.:

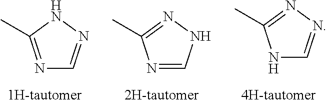

The present invention includes all possible tautomers of the compounds of the present invention as single tautomers, or as any mixture of said tautomers, in any ratio.

Further, the compounds of the present invention can exist as N-oxides, which are defined in that at least one nitrogen of the compounds of the present invention is oxidised. The present invention includes all such possible N-oxides.

The present invention also relates to useful forms of the compounds as disclosed herein, such as metabolites, hydrates, solvates, prodrugs, salts, in particular pharmaceutically acceptable salts, and co-precipitates.

The compounds of the present invention can exist as a hydrate, or as a solvate, wherein the compounds of the present invention contain polar solvents, in particular water, methanol or ethanol for example as structural element of the crystal lattice of the compounds. The amount of polar solvents, in particular water, may exist in a stoichiometric or non-stoichiometric ratio. In the case of stoichiometric solvates, e.g. a hydrate, hemi-, (semi-), mono-, sesqui-, di-, tri-, tetra-, penta- etc. solvates or hydrates, respectively, are possible. The present invention includes all such hydrates or solvates.

Further, the compounds of the present invention can exist in free form, e.g. as a free base, or as a free acid, or as a zwitterion, or can exist in the form of a salt. Said salt may be any salt, either an organic or inorganic addition salt, particularly any pharmaceutically acceptable organic or inorganic addition salt, customarily used in pharmacy.

The term “pharmaceutically acceptable salt” refers to a relatively non-toxic, inorganic or organic acid addition salt of a compound of the present invention. For example, see S. M. Berge, et al. “Pharmaceutical Salts,” J. Pharm. Sci. 1977, 66, 1-19.

The present invention includes all possible salts of the compounds of the present invention as single salts, or as any mixture of said salts, in any ratio. Furthermore, the present invention includes all possible crystalline forms, or polymorphs, of the compounds of the present invention, either as single polymorphs, or as a mixture of more than one polymorphs, in any ratio.

In accordance with a first aspect, the present invention relates to compounds of general formula (I):

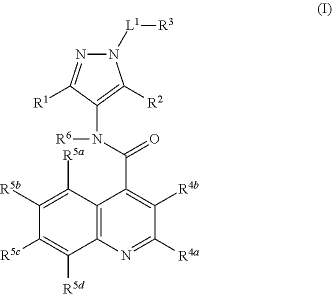

in which:
R¹ represents a C₁-C₃-alkyl-, halo-C₁-C₃-alkyl-, cyano-, —C(═O)O—R¹⁰ or —C(═O)N(R^{10a})R^{10b} group;
R² represents a C₁-C₃-alkyl-, halo-C₁-C₃-alkyl-, cyano-, —C(═O)O—R¹⁰ or —C(═O)N(R^{10a})R^{10b} group;
R³ represents a group selected from: aryl-, heteroaryl-, C₅-C₆-cycloalkyl-, and 5- to 6-membered heterocycloalkyl-;
   wherein said 5- to 6-membered heterocycloalkyl- group is optionally benzocondensed;
   wherein said aryl-, heteroaryl-, C₅-C₆-cycloalkyl-, and 5- to 6-membered heterocycloalkyl- group is optionally substituted, one or more times, identically or differently, with -(L²)ₚ-R⁷;
   and wherein two -(L²)ₚ-R⁷ groups, if being present ortho to each other on an aryl- or heteroaryl- group optionally form a bridge selected from: *—C₃-C₈-alkylene-*, *—O(CH₂)₂O—*, *—O(CH₂)O—*, *—O(CF₂)O—*, *—CH₂C(R^{10a})(R^{10b})O—*, *—C(═O)N(R^{10a})CH₂—*, *—N(R^{10a})C(═O)CH₂O—*, *—NHC(═O)NH—*; wherein each * represents the point of attachment to said aryl- or heteroaryl- group;
R^{4a} represents a hydrogen atom or a halogen atom or a group selected from: cyano-, hydroxy-, C₁-C₃-alkyl-, halo-C₁-C₃-alkyl-, C₁-C₃-alkoxy-, halo-C₁-C₃-alkoxy-, C₃—C-cycloalkyl-, 4- to 7-membered heterocycloalkyl-, —C(═O)—OR¹⁰, —C(═O)N(R^{10a})R^{10b}, —C(═O)—N(R^{10a})—S(═O)₂—R¹⁰, —SR¹⁰, —S(═O)—R¹⁰, —S(═NR¹¹)—R¹⁰, —S(═O)₂—R¹⁰, —S(═O)₂—N(R^{10a})R^{10b}, —S(═O)(═NR¹¹)—R¹⁰, —N(R^{10a})R^{10b};
R^{4b} represents a hydrogen atom or a group selected from: C₁-C₃-alkoxy-, C₁-C₃-alkyl-, cyano-;
   or
R^{4a} and together R^{4b} form a —C₃-C₅-alkylene- group;
R^{5a}, R^{5b}, R^{5c}, R^{5d}
   independently from each other represent a hydrogen atom, a halogen atom or a group selected from:
   cyano-, —NO₂, C₁-C₃-alkyl-, halo-C₁-C₃-alkyl-, C₁-C₃-alkoxy-, halo-C₁-C₃-alkoxy-, phenyl-, heteroaryl-, —C(═O)R¹⁰, —C(═O)N(H)R¹⁰, —C(═O)N(R^{10a})R^{10b}, —C(═O)O—R¹⁰, —N(R^{10a})R^{10b}, —N(H)C(═O)R¹⁰, —N(R^{10a})C(═O)R^{10b}, —N(H)C(═O)N(R^{10a})R^{10b}, —N(R^{10a})C(═O)N(R^{10b})R^{10c}, —N(R^{10a})C(═O)C(═O)N(R^{10b})R^{10c}, —N(H)C(═O)OR¹⁰, —N(R^{10a})C(═O)OR^{10b}, —N(H)S(═O)₂R¹⁰, —N(R^{10a})S(═O)₂R^{10b}, —OR¹⁰, —O(C═O)R¹⁰, —O(C═O)N(R^{10a})R^{10b}, —O(C═O)OR¹⁰, —SR¹⁰, —S(═O)R¹⁰, —S(═O)₂R¹⁰, —S(═O)₂N(H)R¹⁰, —S(═O)₂N(R^{10a})R^{10b} or —S(═O)(═NR^{10a})R^{10b},
   said phenyl- or heteroaryl- group being optionally substituted one or more times, identically or differently, with a group selected from: halo-, cyano-, C₁-C₃-alkyl-, halo-C₁-C₃-alkyl-, C₁-C₃-alkoxy-;
   R⁶ represents a hydrogen atom or group selected from: C₁-C₃-alkyl-, C₁-C₃-alkoxy-(L²)-, hydroxy-C₁-C₃-alkyl-, aryl-(L²)-, heteroaryl-(L²)-;
R⁷ represents a group selected from: oxo, C₁-C₆-alkyl-, C₃-C₇-cycloalkyl-, 4- to 7-membered heterocycloalkyl-, halo-C₁-C₄-alkyl-, hydroxy-C₁-C₄-alkyl-, cyano-C₁-C₄-alkyl-, C₂-C₄-alkenyl-, C₂-C₄-alkynyl-, C₁-C₄-alkoxy-, halo-C₁-C₄-alkoxy-, —OH, —CN, halo-, —C(═O)R⁸, —C(═O)—O—R⁸, —C(═O)N(R^{8a})R^{8b}, —N(R^{10a})R^{10b}, —S(═O)₂R⁸, —S(═O)(═NR¹¹)—R¹⁰, phenyl-, 5- to 6-membered heteroaryl-;
R⁸ represents a hydrogen atom or a C₁-C₆-alkyl-, halo-C₁-C₃-alkyl-, cyano-C₁-C₄-alkyl-, C₁-C₃-alkoxy-C₁-C₃-alkyl-, C₃-C₇-cycloalkyl-, phenyl-, 5- to 6-membered heteroaryl- or benzyl- group;
R^{8a}, R^{8b}
   represent, independently from each other, a hydrogen atom, or a C₁-C₁₀-alkyl-, C₃-C₇-cycloalkyl-, (C₃-C₇-cycloalkyl)-(L³)-, C₃-C₆-alkenyl-, C₃-C₆-alkynyl-, 4- to 10-membered heterocycloalkyl-,
   (4- to 10-membered heterocycloalkyl)-(L³)-, phenyl-, heteroaryl-, phenyl-(L³)-, (phenyl)-O-(L³)-, heteroaryl-(L³)-, or
   (aryl)-(4- to 10-membered heterocycloalkyl)- group;
   said C₁-C₁₀-alkyl-, C₃-C₇-cycloalkyl-, (C₃-C₇-cycloalkyl)-(L³)-, C₃-C₆-alkenyl-, C₃-C₆-alkynyl-, 4- to 10-membered heterocycloalkyl-, (4- to 10-membered heterocycloalkyl)-(L³)-, phenyl-, heteroaryl-, phenyl-(L³)-, (phenyl)-O-(L³)-, heteroaryl-(L³)-, and (aryl)-(4- to 10-membered heterocycloalkyl)- group being optionally substituted one or more times, identically or differently, with R⁹;
      or
R^{8a} and R^{8b}, together with the nitrogen atom they are attached to, represent a 4- to 10-membered heterocycloalkyl-group, said 4- to 10-membered heterocycloalkyl- group being optionally substituted one or more times, identically or differently, with R⁹;
R⁹ represents a halogen atom, or a oxo, C₁-C₃-alkyl-, halo-C₁-C₃-alkyl-, hydroxy-C₁-C₃-alkyl-, —CN, —C(═O)R¹⁰, —C(═O)N(H)R¹⁰, —C(═O)N(R^{10a})R^{10b}, —C(═O)O—R¹⁰, —N(R^{10a})R^{10b}, —NO₂, —N(H)C(═O)R¹⁰, —N(R^{10a})C(═O)R^{10b}, —N(H)C(═O)N(R^{10a})R^{10b}, —N(R^{10a})C(═O)N(R^{10b})R^{10c}, —N(H)C(═O)OR¹⁰, —N(R^{10a})C(═O)OR^{10b}, —N(H)S(═O)₂R¹⁰, —N(R^{10a})S(═O)₂R^{10b}, —OR¹⁰, —O(C═O)R¹⁰, —O(C═O)N(R^{10a})R^{10b}, —O(C═O)OR¹⁰, —SR¹⁰, —S(═O)R¹⁰, —S(═O)₂R¹⁰, —S(═O)₂N(H)R¹⁰, —S(═O)₂N(R^{10a})R^{10b}, —S(═O)(═NR^{10a})R^{10b} or a tetrazolyl-group;
   or
   two R⁹ groups present ortho to each other on a phenyl- or heteroaryl-ring form a bridge selected from: *—C₃-C₅-alkylene-*, *—O(CH₂)₂O—*, *—O(CH₂)O—*, *—O(CF₂)O—*, *—CH₂C(R^{10a})(R^{10b})O—*, *—C(═O)N(R^{10a})CH₂—*, *—N(R^{10a})C(═O)CH₂O—*, *—NHC(═O)NH—*; wherein each * represents the point of attachment to said phenyl- or heteroaryl- ring;
R¹⁰, R^{10a}, R^{10b}, R^{10c}
   represent, independently from each other, a hydrogen atom or a group selected from: C₁-C₃-alkyl-, halo-C₁-C₃-alkyl-, hydroxy-C₁-C₃-alkyl-, C₁-C₃-alkoxy-C₁-C₃-alkyl-, C₃-C₇-cycloalkyl-, said C₁-C₃-alkyl- group being optionally substituted once with —N(R¹²)R^{12a};
      or
R^{10a} and R^{10b}, together with the nitrogen atom they are attached to, represent a 4- to 7-membered heterocycloalkyl- group, said 4- to 7-membered heterocycloalkyl-group being optionally substituted one or more times, identically or differently, with R¹³;
   R¹¹ represents a hydrogen atom or a cyano-, C₁-C₃-alkyl-, —C(═O)R¹⁰, —C(═O)N(H)R¹⁰, —C(═O)N(R^{10a})R^{10b} or —C(═O)O—R¹⁰ group;
R¹², R^{12a}
   represent, independently from each other, a hydrogen atom or a C₁-C₃-alkyl- group,
   or,
R¹², R^{12a},
   together with the nitrogen atom they are attached to, represent a 4- to 7-membered heterocycloalkyl- group;
R¹³ represents a halogen atom or a cyano, hydroxy, oxo, C₁-C₃-alkyl-, trifluoromethyl-, —C(═O)R¹⁰ or —C(═O)O—R¹⁰ group;
L¹ represents a group selected from: —C₁-C₄-alkylene-, —CH₂—CH═CH—, —C(phenyl)(H)—, —CH₂—CH₂—O—, —CH₂—C(═O)—N(H)—, —CH₂—C(═O)—N(R^{10a})—;
L² represents a group selected from: —CH₂—, —CH₂—CH₂—, —CH₂—CH₂—CH₂—;
L³ represents a —C₁-C₆-alkylene- group;
p is an integer of 0 or 1;
   or a tautomer, a stereoisomer, an N-oxide, a hydrate, a solvate, or a salt thereof, or a mixture of same.

In a preferred embodiment, the invention relates to compounds of formula (I), supra, wherein R¹ represents a C₁-C₃-alkyl-, halo-C₁-C₃-alkyl- or cyano- group.

In another preferred embodiment, the invention relates to compounds of formula (I), supra, wherein R¹ represents a C₁-C₃-alkyl-, halo-C₁-C₃-alkyl- or cyano- group, and wherein at least one of R¹ and R² is different from iso-propyl-.

In another preferred embodiment, the invention relates to compounds of formula (I), supra, wherein at east one of R¹ and R² is different from iso-propyl-.

In another preferred embodiment, the invention relates to compounds of formula (I), supra, wherein R¹ represents a C₁-C₃-alkyl- or halo-C₁-C₃-alkyl-group.

In another preferred embodiment, the invention relates to compounds of formula (I), supra, wherein R¹ represents a C₁-C₃-alkyl- or halo-C₁-C₃-alkyl-group, and wherein at least one of R¹ and R² is different from iso-propyl-.

In another preferred embodiment, the invention relates to compounds of formula (I), supra, wherein R¹ represents a C₁-C₃-alkyl- or trifluoromethyl-group.

In another preferred embodiment, the invention relates to compounds of formula (I), supra, wherein R¹ represents a C₁-C₃-alkyl- or trifluoromethyl-group, and wherein at least one of R¹ and R² is different from iso-propyl-.

In another preferred embodiment, the invention relates to compounds of formula (I), supra, wherein R¹ represents a C₁-C₃-alkyl- group.

In another preferred embodiment, the invention relates to compounds of formula (I), supra, wherein R¹ represents a C₁-C₃-alkyl- group, and wherein at least one of R¹ and R² is different from iso-propyl-.

In another preferred embodiment, the invention relates to compounds of formula (I), supra, wherein R¹ represents a methyl-, ethyl- or trifluoromethyl-group.

In another preferred embodiment, the invention relates to compounds of formula (I), supra, wherein R¹ represents a methyl- or trifluoromethyl- group.

In another preferred embodiment, the invention relates to compounds of formula (I), supra, wherein R¹ represents a methyl- group.

In another preferred embodiment, the invention relates to compounds of formula (I), supra, wherein R¹ represents a trifluoromethyl- group.

In another preferred embodiment, the invention relates to compounds of formula (I), supra, wherein R¹ represents a C₁-C₃-alkyl-, fluoro-C₁-C₃-alkyl-, cyano-, —C(═O)O—R¹⁰ or —C(═O)N(R^{10a})R^{10b} group.

In another preferred embodiment, the invention relates to compounds of formula (I), supra, wherein R¹ represents a C₁-C₃-alkyl-, fluoro-C₁-C₃-alkyl-, cyano-, —C(═O)O—R¹⁰ or —C(═O)N(R^{10a})R^{10b} group, and wherein at least one of R¹ and R² is different from iso-propyl-.

In another preferred embodiment, the invention relates to compounds of formula (I), supra, wherein R¹ represents a C₁-C₃-alkyl-, fluoro-C₁-C₃-alkyl- or cyano- group.

In another preferred embodiment, the invention relates to compounds of formula (I), supra, wherein R¹ represents a C₁-C₃-alkyl-, fluoro-C₁-C₃-alkyl- or cyano- group, and wherein at least one of R¹ and R² is different from iso-propyl-.

In another preferred embodiment, the invention relates to compounds of formula (I), supra, wherein R¹ represents a —C(═O)N(R^{10a})R^{10b} group.

In another preferred embodiment, the invention relates to compounds of formula (I), supra, wherein R¹ represents a C₁-C₃-alkyl-, trifluoromethyl- or cyano- group.

In another preferred embodiment, the invention relates to compounds of formula (I), supra, wherein R¹ represents a C₁-C₃-alkyl-, trifluoromethyl- or cyano- group, and wherein at least one of R¹ and R² is different from iso-propyl-.

In a preferred embodiment, the invention relates to compounds of formula (I), supra, wherein R² represents a C₁-C₃-alkyl-, halo-C₁-C₃-alkyl- or cyano- group.

In a preferred embodiment, the invention relates to compounds of formula (I), supra, wherein R² represents a C₁-C₃-alkyl-, halo-C₁-C₃-alkyl- or cyano- group, and wherein at east one of R¹ and R² is different from iso-propyl-.

In another preferred embodiment, the invention relates to compounds of formula (I), supra, wherein R² represents a C₁-C₃-alkyl- or halo-C₁-C₃-alkyl-group.

In another preferred embodiment, the invention relates to compounds of formula (I), supra, wherein R² represents a C₁-C₃-alkyl- or halo-C₁-C₃-alkyl-group, and wherein at least one of R¹ and R² is different from iso-propyl-.

In another preferred embodiment, the invention relates to compounds of formula (I), supra, wherein R² represents a C₁-C₃-alkyl- or fluoro-C₁-C₃-alkyl-group.

In another preferred embodiment, the invention relates to compounds of formula (I), supra, wherein R² represents a C₁-C₃-alkyl- or fluoro-C₁-C₃-alkyl-group, and wherein at least one of R¹ and R² is different from iso-propyl-.

In another preferred embodiment, the invention relates to compounds of formula (I), supra, wherein R² represents a C₁-C₃-alkyl- or trifluoromethyl-group.

In another preferred embodiment, the invention relates to compounds of formula (I), supra, wherein R² represents a C₁-C₃-alkyl- or trifluoromethyl-group, and wherein at least one of R¹ and R² is different from iso-propyl-.

In another preferred embodiment, the invention relates to compounds of formula (I), supra, wherein R² represents a C₁-C₃-alkyl- group.

In another preferred embodiment, the invention relates to compounds of formula (I), supra, wherein R² represents a C₁-C₃-alkyl- group, and wherein at least one of R¹ and R² is different from iso-propyl-.

In another preferred embodiment, the invention relates to compounds of formula (I), supra, wherein R² represents a methyl-, ethyl- or trifluoromethyl-group.

In another preferred embodiment, the invention relates to compounds of formula (I), supra, wherein R² represents a methyl- or trifluoromethyl- group.

In another preferred embodiment, the invention relates to compounds of formula (I), supra, wherein R² represents a methyl- group.

In another preferred embodiment, the invention relates to compounds of formula (I), supra, wherein R² represents a trifluoromethyl- group.

In another preferred embodiment, the invention relates to compounds of formula (I), supra, wherein R² represents a C₁-C₃-alkyl-, fluoro-C₁-C₃-alkyl-, —C(═O)O—R¹⁰ or —C(═O)N(R^{10a})R^{10b} group.

In another preferred embodiment, the invention relates to compounds of formula (I), supra, wherein R² represents a C₁-C₃-alkyl-, fluoro-C₁-C₃-alkyl-, —C(═O)O—R¹⁰ or —C(═O)N(R^{10a})R^{10b} group, and wherein at least one of R¹ and R² is different from iso-propyl-.

In another preferred embodiment, the invention relates to compounds of formula (I), supra, wherein R² represents a C₁-C₃-alkyl-, fluoro-C₁-C₃-alkyl- or —C(═O)N(R^{10a})R^{10b} group.

In another preferred embodiment, the invention relates to compounds of formula (I), supra, wherein R² represents a C₁-C₃-alkyl-, fluoro-C₁-C₃-alkyl- or —C(═O)N(R^{10a})R^{10b} group, and wherein at least one of R¹ and R² is different from iso-propyl-.

In another preferred embodiment, the invention relates to compounds of formula (I), supra, wherein R² represents a —C(═O)N(R^{10a})R^{10b} group.

In another preferred embodiment, the invention relates to compounds of formula (I), supra, wherein R¹ represents a C₁-C₃-alkyl- group, and wherein R² represents a methyl-, ethyl- or trifluoromethyl- group.

In another preferred embodiment, the invention relates to compounds of formula (I), supra, wherein R¹ represents a methyl-, ethyl- or trifluoromethyl-group, and wherein R² represents a C₁-C₃-alkyl- group.

In another preferred embodiment, the invention relates to compounds of formula (I), supra, wherein R¹ represents a C₁-C₃-alkyl- group, and wherein R² represents a methyl- or trifluoromethyl- group.

In another preferred embodiment, the invention relates to compounds of formula (I), supra, wherein R¹ represents a methyl- or trifluoromethyl- group, and wherein R² represents a C₁-C₃-alkyl- group.

In another preferred embodiment, the invention relates to compounds of formula (I), supra, wherein R¹ represents a methyl- or trifluoromethyl- group, and wherein R² represents a methyl- group.

In another preferred embodiment, the invention relates to compounds of formula (I), supra, wherein R² represents a methyl- or trifluoromethyl- group, and wherein R¹ represents a methyl- group.

In another preferred embodiment, the invention relates to compounds of formula (I), supra, wherein R¹ and R² each represent a methyl- group.

In another preferred embodiment, the invention relates to compounds of formula (I), supra, wherein R¹ represents a C₁-C₃-alkyl-, fluoro-C₁-C₃-alkyl-, cyano-, —C(═O)O—R¹⁰ or —C(═O)N(R^{10a})R^{10b} group, and wherein R² represents a C₁-C₃-alkyl-, fluoro-C₁-C₃-alkyl- or —C(═O)N(R^{10a})R^{10b} group.

In another preferred embodiment, the invention relates to compounds of formula (I), supra, wherein R¹ represents a C₁-C₃-alkyl-, fluoro-C₁-C₃-alkyl-, cyano-, —C(═O)O—R¹⁰ or —C(═O)N(R^{10a})R^{10b} group, and wherein R² represents a C₁-C₃-alkyl-, fluoro-C₁-C₃-alkyl- or —C(═O)N(R^{10a})R^{10b} group, and wherein at east one of R¹ and R² is different from iso-propyl-.

In another preferred embodiment, the invention relates to compounds of formula (I), supra, wherein R¹ represents a C₁-C₃-alkyl-, fluoro-C₁-C₃-alkyl-, cyano-, —C(═O)O—R¹⁰ or —C(═O)N(R^{10a})R^{10b} group, and wherein R² represents a methyl-, ethyl- or trifluoromethyl- group.

In another preferred embodiment, the invention relates to compounds of formula (I), supra, wherein R¹ represents a C₁-C₃-alkyl-, fluoro-C₁-C₃-alkyl-, cyano-, —C(═O)O—R¹⁰ or —C(═O)N(R^{10a})R^{10b} group, and wherein R² represents a methyl- group.

In another preferred embodiment, the invention relates to compounds of formula (I), supra, wherein R¹ represents a C₁-C₃-alkyl-, trifluoromethyl- or cyano- group, and wherein R² represents a methyl-, ethyl- or trifluoromethyl-group.

In another preferred embodiment, the invention relates to compounds of formula (I), supra, wherein R¹ represents a C₁-C₃-alkyl-, trifluoromethyl- or cyano- group, and wherein R² represents a methyl- group.

In another preferred embodiment, the invention relates to compounds of formula (I), supra, wherein R³ represents a group selected from: aryl- and heteroaryl-; wherein said group is substituted, one or more times, identically or differently, with -(L²)ₚ-R⁷, and wherein two -(L²)ₚ-R⁷ groups, if being present ortho to each other on said aryl- or heteroaryl- group optionally form a bridge selected from: *—C₃-C₅-alkylene-*, *—O(CH₂)₂O—*, *—O(CH₂)O—*, *—O(CF₂)O—*; wherein each * represents the point of attachment to said aryl- or heteroaryl- group.

In a preferred embodiment, the invention relates to compounds of formula (I), supra, wherein R³ represents a group selected from: aryl- and heteroaryl-; wherein said group is substituted, one or more times, identically or differently, with -(L²)ₚ-R⁷.

In another preferred embodiment, the invention relates to compounds of formula (I), supra, wherein R³ represents a group selected from: phenyl-, C₅-C₆-cycloalkyl-, 5- to 6-membered heterocycloalkyl-, pyridin-3-yl- and pyridin-4-yl-, said 5- to 6-membered heterocycloalkyl- group optionally being benzocondensed; wherein said phenyl-, C₅-C₆-cycloalkyl-, 5- to 6-membered heterocycloalkyl-, pyridin-3-yl- and pyridin-4-yl- group, is substituted, one or more times, identically or differently, with -(L²)ₚ-R⁷; In another preferred embodiment, the invention relates to compounds of formula (I), supra, wherein R³ represents a group selected from: phenyl-, C₅-C₆-cycloalkyl-, 5- to 6-membered heterocycloalkyl-, pyridin-3-yl- and pyridin-4-yl-; wherein said group is substituted, one or more times, identically or differently, with -(L²)ₚ-R⁷.

In another preferred embodiment, the invention relates to compounds of formula (I), supra, wherein R³ represents a group selected from: C₅-C₆-cycloalkyl-, 5- to 6-membered heterocycloalkyl-; wherein said group is substituted, one or more times, identically or differently, with -(L²)ₚ-R⁷.

In another preferred embodiment, the invention relates to compounds of formula (I), supra, wherein R³ represents a group selected from: phenyl-, 5- or 6-membered heteroaryl-; wherein said group is substituted, one or more times, identically or differently, with -(L²)ₚ-R⁷.

In another preferred embodiment, the invention relates to compounds of formula (I), supra, wherein R³ represents a group selected from: phenyl-, pyridin-3-yl- and pyridin-4-yl-; wherein said group is substituted, one or more times, identically or differently, with -(L²)ₚ-R⁷.

In another preferred embodiment, the invention relates to compounds of formula (I), supra, wherein R³ represents a phenyl- group; wherein said phenyl-group is substituted, one or more times, identically or differently, with -(L²)ₚ-R⁷.

In another preferred embodiment, the invention relates to compounds of formula (I), supra, wherein R³ represents a C₅-C₆-cycloalkyl- group; wherein said group is substituted, one or more times, identically or differently, with -(L²)ₚ-R⁷.

In another preferred embodiment, the invention relates to compounds of formula (I), supra, wherein R³ represents a 5- to 6-membered heterocycloalkyl-group; wherein said group is substituted, one or more times, identically or differently, with -(L²)ₚ-R⁷.

In another preferred embodiment, the invention relates to compounds of formula (I), supra, wherein R³ represents a group selected from: aryl-, heteroaryl-, C₅-C₆-cycloalkyl-, and 5- to 6-membered heterocycloalkyl-; wherein said 5- to 6-membered heterocycloalkyl- group is optionally benzocondensed;

wherein said aryl-, heteroaryl-, C₅-C₆-cycloalkyl-, and 5- to 6-membered heterocycloalkyl- group is optionally substituted, one or more times, identically or differently, with -(L²)ₚ-R⁷;
and wherein two -(L²)ₚ-R⁷ groups, if being present ortho to each other on an aryl- or heteroaryl- group optionally form a bridge selected from: *—C₃-C₈-alkylene-*, *—O(CH₂)₂O—*, *—O(CH₂)O—; wherein each * represents the point of attachment to said aryl- or heteroaryl- group.

In another preferred embodiment, the invention relates to compounds of formula (I), supra, wherein R³ represents a group selected from: aryl- or 5- to 6-membered heteroaryl- or piperidinyl-;

wherein said aryl- or 5- to 6-membered heteroaryl- or piperidinyl- group is optionally substituted, one or more times, identically or differently, with -(L²)ₚ-R⁷.

In another preferred embodiment, the invention relates to compounds of formula (I), supra, wherein R³ represents an aryl- group;

wherein said aryl- group is optionally substituted, one or more times, identically or differently, with -(L²)ₚ-R⁷.

In another preferred embodiment, the invention relates to compounds of formula (I), supra, wherein R³ represents a 5- to 6-membered heteroaryl-group;

wherein said 5- to 6-membered heteroaryl- group is optionally substituted, one or more times, identically or differently, with -(L²)ₚ-R⁷.

In another preferred embodiment, the invention relates to compounds of formula (I), supra, wherein R³ represents a piperidinyl- group;

wherein said piperidinyl- group is optionally substituted, one or more times, identically or differently, with -(L²)ₚ-R⁷.

In another preferred embodiment, the invention relates to compounds of formula (I), supra, wherein R³ represents a group selected from: phenyl- or 5- to 6-membered heteroaryl-;

wherein said phenyl- or 5- to 6-membered heteroaryl- group is optionally substituted, one or more times, identically or differently, with -(L²)ₚ-R⁷, or wherein R³ represents a group

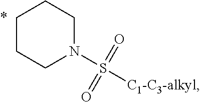

wherein * represents the point of attachment to the rest of the molecule.

In another preferred embodiment, the invention relates to compounds of formula (I), supra, wherein R³ represents a phenyl- group;

wherein said phenyl- group is optionally substituted, one or more times, identically or differently, with -(L²)ₚ-R⁷.

In another preferred embodiment, the invention relates to compounds of formula (I), supra, wherein R³ represents a group

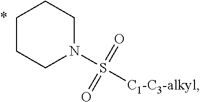

wherein * represents the point of attachment to the rest of the molecule.

In another preferred embodiment, the invention relates to compounds of formula (I), supra, wherein R³ represents a phenyl- group which is optionally substituted, one or more times, identically or differently, with a group selected from C₁-C₃-alkyl-, trifluoromethyl-, cyanomethyl-, methoxymethyl-, C₁-C₃-alkoxy-, trifluoromethoxy-, —CN, fluoro-, chloro-, —C(═O)—C₁-C₃-alkyl, —C(═O)N(R^{8a})R^{8b}, —S(═O)₂—C₁-C₃-alkyl;

or wherein R³ represents a 5- to 6-membered heteroaryl- group which is optionally substituted, one or more times, identically or differently, with a group selected from C₁-C₃-alkyl-, cyclopropyl-, C₁-C₃-alkoxy-, —CN, —C(═O)N(R^{8a})R^{8b}.

In another preferred embodiment, the invention relates to compounds of formula (I), supra, wherein R³ represents a phenyl- group which is optionally substituted, one or more times, identically or differently, with a group selected from C₁-C₃-alkyl-, trifluoromethyl-, cyanomethyl-, methoxymethyl-, C₁-C₃-alkoxy-, trifluoromethoxy-, —CN, fluoro-, chloro-, —C(═O)—C₁-C₃-alkyl, —C(═O)N(R^{8a})R^{8b}, —S(═O)₂—C₁-C₃-alkyl.

In another preferred embodiment, the invention relates to compounds of formula (I), supra, wherein R³ represents a 5- to 6-membered heteroaryl- group which is optionally substituted, one or more times, identically or differently, with a group selected from C₁-C₃-alkyl-, cyclopropyl-, C₁-C₃-alkoxy-, —CN, —C(═O)N(R^{8a})R^{8b}.

In another preferred embodiment, the invention relates to compounds of formula (I), supra, wherein R³ represents a phenyl- group which is substituted, one or more times, identically or differently, with a group selected from methoxy-, —CN, fluoro-,

or wherein R³ represents a pyridyl- or pyrimidyl- group which is substituted once with a group selected from methoxy-, —CN, or wherein R³ represents a 5-membered heteroaryl- group selected from isoxazolyl-, oxadiazolyl- and thienyl-, which is substituted once with a group selected from C₁-C₃-alkyl-, cyclopropyl-, —CN.

In another preferred embodiment, the invention relates to compounds of formula (I), supra, wherein R³ represents a phenyl- group which is substituted, one or more times, identically or differently, with a group selected from methoxy-, —CN, fluoro-.

In another preferred embodiment, the invention relates to compounds of formula (I), supra, wherein R³ represents a pyridyl- or pyrimidyl- group which is substituted once with a group selected from methoxy-, —CN.

In another preferred embodiment, the invention relates to compounds of formula (I), supra, wherein R³ represents a 5-membered heteroaryl- group selected from isoxazolyl-, oxadiazolyl- and thienyl-, which is substituted once with a group selected from C₁-C₃-alkyl-, cyclopropyl-, —CN.

In another preferred embodiment, the invention relates to compounds of formula (I), supra, wherein R³ represents a phenyl- group which is substituted, one or more times, identically or differently, with a group selected from methoxy-, —CN, fluoro-,

or wherein R³ represents a pyridyl- or pyrimidyl- group which is substituted once with a group selected from methoxy-, —CN.

In another preferred embodiment, the invention relates to compounds of formula (I), supra, wherein R³ represents a pyridyl- or pyrimidyl- group which is substituted once with a group selected from methoxy-, —CN.

In another preferred embodiment, the invention relates to compounds of formula (I), supra, wherein R³ represents an isoxazolyl-group which is substituted once with a group selected from C₁-C₃-alkyl-, cyclopropyl-.

In a preferred embodiment, the invention relates to compounds of formula (I), supra, wherein R^{4a} represents a hydrogen atom or a halogen atom or a group selected from: cyano-, hydroxy-, methyl-, ethyl-, -trifluoromethyl-, methoxy-, ethoxy-, C₃-C₇-cycloalkyl-, 4- to 7-membered heterocycloalkyl-, —C(═O)N(R^{10a})R^{10b}, —N(R^{10a})R^{10b}.

In another preferred embodiment, the invention relates to compounds of formula (I), supra, wherein R^{4a} represents a hydrogen atom or a halogen atom or a group selected from: cyano-, hydroxy-, C₁-C₃-alkyl-, halo-C₁-C₃-alkyl-, C₁-C₃-alkoxy-, —C(═O)N(R^{10a})R^{10b}, —N(R^{10a})R^{10b}.

In another preferred embodiment, the invention relates to compounds of formula (I), supra, wherein R^{4a} represents a hydrogen atom or a halogen atom or a group selected from: cyano-, hydroxy-, methyl-, ethyl-, -trifluoromethyl-, methoxy-, ethoxy-, —C(═O)N(R^{10a})R^{10b}, —N(R^{10a})R^{10b}In another preferred embodiment, the invention relates to compounds of formula (I), supra, wherein R^{4a} represents a group selected from: C₃—C-cycloalkyl-, 4- to 7-membered heterocycloalkyl-.

In another preferred embodiment, the invention relates to compounds of formula (I), supra, wherein R^{4a} represents C₃-C₄-cycloalkyl-.

In another preferred embodiment, the invention relates to compounds of formula (I), supra, wherein R^{4a} represents a hydrogen atom or a group selected from: methyl-, ethyl-, -trifluoromethyl-, methoxy-, ethoxy-, —C(═O)N(R^{10a})R^{10b}, —N(R^{10a})R^{10b}.

In another preferred embodiment, the invention relates to compounds of formula (I), supra, wherein R^{4a} represents a hydrogen atom or a group selected from: methyl-, -trifluoromethyl-, methoxy-, —C(═O)NH₂.

In another preferred embodiment, the invention relates to compounds of formula (I), supra, wherein R^{4a} represents a hydrogen atom or a halogen atom or a group selected from: cyano-, hydroxy-, C₁-C₃-alkyl-, fluoro-C₁-C₃-alkyl-, C₁-C₃-alkoxy-, fluoro-C₁-C₃-alkoxy-, C₃-C₇-cycloalkyl-, 4- to 7-membered heterocycloalkyl-, —C(═O)—OR¹⁰, —C(═O)N(R^{10a})R^{10b}, —C(═O)—N(R^{10a})—S(═O)₂—R¹⁰, —SR¹⁰, —S(═O)—R¹⁰, —S(═NR¹¹)—R¹⁰, —S(═O)₂—R¹⁰, —S(═O)₂—N(R^{10a})R^{10b}, —S(═O)(═NR¹¹)—R¹⁰, —N(R^{10a})R^{10b}.

In another preferred embodiment, the invention relates to compounds of formula (I), supra, wherein R^{4a} represents a group selected from: C₁-C₃-alkyl-, fluoro-C₁-C₃-alkyl-, C₁-C₃-alkoxy-, C₃-C₅-cycloalkyl, —C(═O)N(R^{10a})R^{10b}, —SR¹⁰, —S(═O)—R¹⁰, —S(═NR¹¹)—R¹⁰, —S(═O)₂—R¹⁰, —S(═O)₂—N(R^{10a})R^{10b}, —N(R^{10a})R^{10b}.

In another preferred embodiment, the invention relates to compounds of formula (I), supra, wherein R^{4a} represents a group selected from: C₁-C₃-alkyl-, fluoro-C₁-C₃-alkyl-, C₁-C₃-alkoxy-, C₃-C₅-cycloalkyl, —C(═O)N(R^{10a})R^{10b}.

In another preferred embodiment, the invention relates to compounds of formula (I), supra, wherein R^{4a} represents a group selected from: iso-propyl-, trifluoromethyl-, methoxy-, cyclopropyl-, —C(═O)—NH₂.

In another preferred embodiment, the invention relates to compounds of formula (I), supra, wherein R^{4a} represents an iso-propyl- group.

In another preferred embodiment, the invention relates to compounds of formula (I), supra, wherein R^{4a} represents a methoxy- group.

In another preferred embodiment, the invention relates to compounds of formula (I), supra, wherein R^{4a} represents a trifluoromethyl- group.

In another preferred embodiment, the invention relates to compounds of formula (I), supra, wherein R^{4a} represents a —C(═O)—NH₂ group.

In another preferred embodiment, the invention relates to compounds of formula (I), supra, wherein R^{4a} represents a cyclopropyl- group.

In a preferred embodiment, the invention relates to compounds of formula (I), supra, wherein R^{4b} represents a hydrogen atom.

In another preferred embodiment, the invention relates to compounds of formula (I), supra, wherein R^{4b} represents a group selected from: C₁-C₃-alkoxy-, C₁-C₃-alkyl-, cyano-.

In another preferred embodiment, the invention relates to compounds of formula (I), supra, wherein R^{4a} and together R^{4b} form a —C₃-C₅-alkylene- group.

In another preferred embodiment, the invention relates to compounds of formula (I), supra, wherein R^{4b} represents a group selected from: C₁-C₃-alkoxy-, C₁-C₃-alkyl-, cyano-, or wherein R^{4a} and together R^{4b} form a —C₃-C₅-alkylene- group.

In a preferred embodiment, the invention relates to compounds of formula (I), supra, wherein R^{5a}, R^{5b}, R^{5c}, R^{5d} independently from each other represent a hydrogen atom, a halogen atom or a group selected from:

cyano-, —NO₂, C₁-C₃-alkyl-, halo-C₁-C₃-alkyl-, C₁-C₃-alkoxy-, halo-C₁-C₃-alkoxy-, phenyl-, heteroaryl-, —C(═O)R¹⁰, —C(═O)N(H)R¹⁰, —C(═O)N(R^{10a})R^{10b}, —C(═O)O—R¹⁰, —N(R^{10a})R^{10b}, —N(H)C(═O)R¹⁰, —N(R^{10a})C(═O)R^{10b}, —N(R^{10a})C(═O)C(═O)N(R^{10b})R^{10c}, —N(H)S(═O)₂R¹⁰;
said phenyl- or heteroaryl- group being optionally substituted one or more times with a C₁-C₃-alkyl- group.

In another preferred embodiment, the invention relates to compounds of formula (I), supra, wherein R^{5a}, R^{5b}, R^{5c}, R^{5d} independently from each other represent a hydrogen atom, a halogen atom or a group selected from:

cyano-, —NO₂, C₁-C₃-alkyl-, fluoro-C₁-C₃-alkyl-, C₁-C₃-alkoxy-, fluoro-C₁-C₃-alkoxy-, phenyl-, heteroaryl-, —C(═O)R¹⁰, —C(═O)N(H)R¹⁰, —C(═O)N(R^{10a})R^{10b}, —C(═O)O—R¹⁰, —N(R^{10a})R^{10b}, —N(H)C(═O)R¹⁰, —N(R^{10a})C(═O)R^{10b}, —N(R^{10a})C(═O)C(═O)N(R^{10b})R^{10c}, —N(H)S(═O)₂R¹⁰;
said phenyl- or heteroaryl- group being optionally substituted one or more times with a C₁-C₃-alkyl- group.

In another preferred embodiment, the invention relates to compounds of formula (I), supra, wherein R^{5a}, R^{5b}, R^{5c}, R^{5d} independently from each other represent a hydrogen atom, a halogen atom or a group selected from: —NO₂, C₁-C₃-alkyl-, fluoro-C₁-C₃-alkyl-, C₁-C₃-alkoxy-, fluoro-C₁-C₃-alkoxy-, —C(═O)N(R^{10a})R^{10b}, —C(═O)O—R¹⁰, —N(R^{10a})R^{10b}, —N(H)C(═O)R¹⁰, —N(R^{10a})C(═O)R^{10b}.

In another preferred embodiment, the invention relates to compounds of formula (I), supra, wherein R^{5a}, R^{5b}, R^{5c}, R^{5d} independently from each other represent a hydrogen atom, a halogen atom or a group selected from: methyl-, trifluoromethyl-, methoxy-, trifluoromethoxy-, —C(═O)O—R¹⁰, —NH₂, —N(H)C(═O)R¹⁰, and wherein R¹⁰ represents methyl-.

In another preferred embodiment, the invention relates to compounds of formula (I), supra, wherein R^{5a}, R^{5b}, R^{5c}, R^{5d} independently from each other represent a hydrogen atom, a halogen atom or a group selected from: cyano-, C₁-C₃-alkyl-, fluoro-C₁-C₃-alkyl-, C₁-C₃-alkoxy-, fluoro-C₁-C₃-alkoxy-, phenyl-, heteroaryl-, —C(═O)R¹⁰, —C(═O)N(H)R¹⁰, —C(═O)N(R^{10a})R^{10b}, —C(═O)O—R¹⁰, —N(R^{10a})R^{10b}, —N(H)C(═O)R¹⁰, —N(R^{10a})C(═O)R^{10b}, —OR¹⁰, said phenyl- or heteroaryl- group being optionally substituted one or more times, identically or differently, with a group selected from:

halo-, cyano-, methyl-, ethyl-, trifluoromethyl-, methoxy, ethoxy-.

In another preferred embodiment, the invention relates to compounds of formula (I), supra, wherein R^{5a}, R^{5b}, R^{5c}, R^{5d} independently from each other represent a hydrogen atom, a halogen atom or a group selected from: cyano-, C₁-C₃-alkyl-, C₁-C₃-alkoxy-, —N(R^{10a})R^{10b}, —OR¹⁰.

In another preferred embodiment, the invention relates to compounds of formula (I), supra, wherein R^{5a}, R^{5b}, R^{5c}, R^{5d} independently from each other represent a hydrogen atom, a halogen atom or a group selected from: cyano-, C₁-C₃-alkyl-, C₁-C₃-alkoxy-, —N(R^{10a})R^{10b}, —OH.

In another preferred embodiment, the invention relates to compounds of formula (I), supra, wherein R^{5a}, R^{5c}, R^{5d} independently from each other represent hydrogen atom, a fluoro atom or a chloro atom, and wherein R^{5b} represents a hydrogen atom, a fluoro atom, a chloro atom, a bromo atom or a group selected from: cyano-, methyl-, methoxy-, —N(H)—CH₂—CH₂—OCH₃, and N-piperidinyl-.

In another preferred embodiment, the invention relates to compounds of formula (I), supra, wherein R^{5a}, R^{5c}, R^{5d} independently from each other represent hydrogen atom, a fluoro atom or a chloro atom.

In another preferred embodiment, the invention relates to compounds of formula (I), supra, wherein R^{5b} represents a hydrogen atom, a fluoro atom, a chloro atom, a bromo atom or a group selected from: cyano-, methyl-, methoxy-, —N(H)—CH₂—CH₂—OCH₃, and N-piperidinyl-.

In another preferred embodiment, the invention relates to compounds of formula (I), supra, wherein R^{5a}, R^{5b}, R^{5c}, R^{5d} independently from each other represent a hydrogen atom, a fluoro atom or a chloro atom.

In another preferred embodiment, the invention relates to compounds of formula (I), supra, wherein R⁶ represents a hydrogen atom or group selected from: C₁-C₃-alkyl-, C₁-C₃-alkoxy-(L²)-, hydroxy-C₁-C₃-alkyl-, aryl-(L²)-, heteroaryl-(L²)-, and wherein L² represents —CH₂— or —CH₂CH₂—.

In another preferred embodiment, the invention relates to compounds of formula (I), supra, wherein R⁶ represents a hydrogen atom or group selected from: C₁-C₃-alkyl-, C₁-C₃-alkoxy-(L²)-, hydroxy-C₁-C₃-alkyl.

In another preferred embodiment, the invention relates to compounds of formula (I), supra, wherein R⁶ represents a hydrogen atom or group selected from: C₁-C₃-alkyl-, C₁-C₃-alkoxy-(L²)-, hydroxy-C₁-C₃-alkyl, and wherein L² represents —CH₂— or —CH₂CH₂—.

In another preferred embodiment, the invention relates to compounds of formula (I), supra, wherein R⁶ represents a hydrogen atom or group selected from: aryl-(L²)-, heteroaryl-(L²)-.

In another preferred embodiment, the invention relates to compounds of formula (I), supra, wherein R⁶ represents a hydrogen atom or group selected from: aryl-(L²)-, heteroaryl-(L²)-, and wherein L² represents —CH₂— or —CH₂CH₂—.

In another preferred embodiment, the invention relates to compounds of formula (I), supra, wherein R⁶ represents a hydrogen atom.

In another preferred embodiment, the invention relates to compounds of formula (I), supra, wherein R⁷ represents a group selected from: oxo, C₁-C₃-alkyl-, fluoro-C₁-C₃-alkyl-, C₁-C₃-alkoxy-, fluoro-C₁-C₃-alkoxy-, —OH, —CN, halo-, —C(═O)R⁸, —C(═O)—O—R⁸, —C(═O)N(R^{8a})R^{8b}, —S(═O)₂R⁸, phenyl-.

In another preferred embodiment, the invention relates to compounds of formula (I), supra, wherein R⁷ represents a group selected from: oxo, C₁-C₄-alkyl-, C₃-C₇-cycloalkyl-, 4- to 7-membered heterocycloalkyl-, fluoro-C₁-C₄-alkyl-, hydroxy-C₁-C₄-alkyl-, cyano-C₁-C₄-alkyl-, C₂-C₄-alkenyl-, C₁-C₄-alkoxy-, fluoro-C₁-C₄-alkoxy-, —OH, —CN, halo-, —C(═O)R⁸, —C(═O)—O—R⁸, —C(═O)N(R^{8a})R^{8b}, —N(R^{10a})R^{10b}, —S(═O)₂R⁸, —S(═O)(═NR¹¹)—R¹⁰, phenyl-, 5- to 6-membered heteroaryl-.

In another preferred embodiment, the invention relates to compounds of formula (I), supra, wherein R⁷ represents a group selected from: C₁-C₃-alkyl-, cyclopropyl-, trifluoromethyl-, C₁-C₃-alkoxy-, trifluoromethoxy-, —CN, fluoro-, chloro-, —C(═O)—C₁-C₃-alkyl, —C(═O)N(R^{8a})R^{8b}, —S(═O)₂)—C₁-C₃-alkyl.

In another preferred embodiment, the invention relates to compounds of formula (I), supra, wherein R⁷ represents a group selected from: C₁-C₃-alkyl-, cyclopropyl-, trifluoromethyl-, C₁-C₃-alkoxy-, trifluoromethoxy-, —CN, fluoro-, chloro-, —C(═O)—C₁-C₃-alkyl, —C(═O)N(R^{8a})R^{8b}, —S(═O)₂—C₁-C₃-alkyl.

In another preferred embodiment, the invention relates to compounds of formula (I), supra, wherein R⁷ represents a group selected from: C₁-C₃-alkyl-, trifluoromethyl-, C₁-C₃-alkoxy-, trifluoromethoxy-, —CN, fluoro-, chloro-, —C(═O)—C₁-C₃-alkyl, —C(═O)N(R^{8a})R^{8b}, —S(═O)₂—C₁-C₃-alkyl.

In another preferred embodiment, the invention relates to compounds of formula (I), supra, wherein R⁷ represents a group selected from: C₁-C₃-alkyl-, cyclopropyl-, C₁-C₃-alkoxy-, —CN, —C(═O)N(R^{8a})R^{8b}.

In another preferred embodiment, the invention relates to compounds of formula (I), supra, wherein R⁷ represents a group selected from: C₁-C₃-alkyl-, cyclopropyl-, methoxy-, —CN, fluoro-.

In another preferred embodiment, the invention relates to compounds of formula (I), supra, wherein R⁷ represents a group selected from: methoxy-, —CN, fluoro-.

In another preferred embodiment, the invention relates to compounds of formula (I), supra, wherein R⁷ represents a group selected from: —CN, fluoro-.

In another preferred embodiment, the invention relates to compounds of formula (I), supra, wherein R⁷ represents a group selected from: methoxy-, —CN.

In another preferred embodiment, the invention relates to compounds of formula (I), supra, wherein R⁷ represents a group selected from: methoxy-, fluoro-.

In another preferred embodiment, the invention relates to compounds of formula (I), supra, wherein R⁷ represents a group selected from: C₁-C₃-alkyl-, cyclopropyl-.

In another preferred embodiment, the invention relates to compounds of formula (I), supra, wherein R⁸ represents a hydrogen atom or a C₁-C₆-alkyl- or benzyl- group.

In another preferred embodiment, the invention relates to compounds of formula (I), supra, wherein R⁸ represents a hydrogen atom or a C₁-C₆-alkyl-group.

In another preferred embodiment, the invention relates to compounds of formula (I), supra, wherein R⁸ represents a hydrogen atom or a C₁-C₆-alkyl-, fluoro-C₁-C₃-alkyl-, cyano-C₁-C₄-alkyl-, C₁-C₃-alkoxy-C₁-C₃-alkyl-, C₃—C-cycloalkyl-, phenyl-, 5- to 6-membered heteroaryl- or benzyl- group.

In another preferred embodiment, the invention relates to compounds of formula (I), supra, wherein R⁸ represents a hydrogen atom or a C₁-C₆-alkyl-, C₃—C-cycloalkyl-, phenyl- or benzyl- group.

In another preferred embodiment, the invention relates to compounds of formula (I), supra, wherein R^{8a} and R^{8b}, independently from each other, represent a hydrogen atom, or a C₁-C₆-alkyl-, C₃-C₇-cycloalkyl-, (C₃-C₇-cycloalkyl)-(L³)-, 4- to 10-membered heterocycloalkyl-, (4- to 10-membered heterocycloalkyl)-(L³)-, phenyl-, heteroaryl-, phenyl-(L³)-, (phenyl)-O-(L³)-, heteroaryl-(L³)-, or (aryl)-(4- to 10-membered heterocycloalkyl)- group;

said C₁-C₆-alkyl-, C₃-C₇-cycloalkyl-, (C₃-C₇-cycloalkyl)-(L³)-, 4- to 10-membered heterocycloalkyl-, (4- to 10-membered heterocycloalkyl)-(L³)-, phenyl-, heteroaryl-, phenyl-(L³)-, (phenyl)-O-(L³)-, heteroaryl-(L³)-, and (aryl)-(4- to 10-membered heterocycloalkyl)- group being optionally substituted one or more times, identically or differently, with R⁹;
or wherein R^{8a} and R^{8b}, together with the nitrogen atom they are attached to, represent a 4- to 10-membered heterocycloalkyl-group, said 4- to 10-membered heterocycloalkyl- group being optionally substituted one or more times, identically or differently, with R⁹.

In another preferred embodiment, the invention relates to compounds of formula (I), supra, wherein R^{8a} and R^{8b}, independently from each other, represent a hydrogen atom, or a C₁-C₆-alkyl-, C₃-C₇-cycloalkyl-, (C₃-C₇-cycloalkyl)-(L³)-, 4- to 10-membered heterocycloalkyl-, (4- to 10-membered heterocycloalkyl)-(L³)-, phenyl-, heteroaryl-, phenyl-(L³)-, (phenyl)-O-(L³)-, heteroaryl-(L³)-, or (aryl)-(4- to 10-membered heterocycloalkyl)- group;

said C₁-C₆-alkyl-, C₃-C₇-cycloalkyl-, (C₃-C₇-cycloalkyl)-(L³)-, 4- to 10-membered heterocycloalkyl-, (4- to 10-membered heterocycloalkyl)-(L³)-, phenyl-, heteroaryl-, phenyl-(L³)-, (phenyl)-O-(L³)-, heteroaryl-(L³)-, and (aryl)-(4- to 10-membered heterocycloalkyl)- group being optionally substituted one or more times, identically or differently, with R⁹.

In another preferred embodiment, the invention relates to compounds of formula (I), supra, wherein R^{8a} and R^{8b}, together with the nitrogen atom they are attached to, represent a 4- to 10-membered heterocycloalkyl-group, said 4- to 10-membered heterocycloalkyl- group being optionally substituted one or more times, identically or differently, with R⁹.

In another preferred embodiment, the invention relates to compounds of formula (I), supra, wherein R^{8a} and R^{8b}, independently from each other, represent a hydrogen atom, or a C₁-C₆-alkyl-, C₃-C₇-cycloalkyl-, 4- to 10-membered heterocycloalkyl-, (4- to 10-membered heterocycloalkyl)-(L³)-, phenyl-, heteroaryl-, phenyl-(L³)- or heteroaryl-(L³)- group;

said C₁-C₆-alkyl-, C₃-C₇-cycloalkyl-, 4- to 10-membered heterocycloalkyl-, (4- to 10-membered heterocycloalkyl)-(L³)-, phenyl-, heteroaryl-, phenyl-(L³)-, and heteroaryl-(L³)- group being optionally substituted one or more times, identically or differently, with R⁹;
or wherein R^{8a} and R^{8b}, together with the nitrogen atom they are attached to, represent a 4- to 7-membered heterocycloalkyl-group.

In another preferred embodiment, the invention relates to compounds of formula (I), supra, wherein R^{8a} and R^{8b}, independently from each other, represent a hydrogen atom, or a C₁-C₆-alkyl-, C₃-C₇-cycloalkyl-, 4- to 10-membered heterocycloalkyl-, (4- to 10-membered heterocycloalkyl)-(L³)-, phenyl-, heteroaryl-, phenyl-(L³)- or heteroaryl-(L³)- group;

said C₁-C₆-alkyl-, C₃-C₇-cycloalkyl-, 4- to 10-membered heterocycloalkyl-, (4- to 10-membered heterocycloalkyl)-(L³)-, phenyl-, heteroaryl-, phenyl-(L³)-, and heteroaryl-(L³)- group being optionally substituted one or more times, identically or differently, with R⁹.

In another preferred embodiment, the invention relates to compounds of formula (I), supra, wherein R^{8a} and R^{8b}, together with the nitrogen atom they are attached to, represent a 4- to 7-membered heterocycloalkyl-group.

In another preferred embodiment, the invention relates to compounds of formula (I), supra, wherein R⁸ and R^{8b}, independently from each other, represent a hydrogen atom, or a C₁-C₄-alkyl-, C₃-C₅-cycloalkyl-, 4- to 7-membered heterocycloalkyl-, (4- to 7-membered heterocycloalkyl)-(L³)-, phenyl- or heteroaryl-(L³)- group;

said C₁-C₄-alkyl-, C₃-C₅-cycloalkyl-, 4- to 7-membered heterocycloalkyl-, (4- to 7-membered heterocycloalkyl)-(L³)-, phenyl- or heteroaryl-(L³)- group being optionally substituted one or more times, identically or differently, with R⁹;
or wherein R^{8a} and R^{8b}, together with the nitrogen atom they are attached to, represent a 4- to 7-membered heterocycloalkyl-group.

In another preferred embodiment, the invention relates to compounds of formula (I), supra, wherein R^{8a} and R^{8b}, independently from each other, represent a hydrogen atom, or a C₁-C₄-alkyl-, C₃-C₅-cycloalkyl-, 4- to 7-membered heterocycloalkyl-, (4- to 7-membered heterocycloalkyl)-(L³)-, phenyl- or heteroaryl-(L³)- group;

said C₁-C₄-alkyl-, C₃-C₅-cycloalkyl-, 4- to 7-membered heterocycloalkyl-, (4- to 7-membered heterocycloalkyl)-(L³)-, phenyl- or heteroaryl-(L³)- group being optionally substituted one or more times, identically or differently, with R⁹.

In another preferred embodiment, the invention relates to compounds of formula (I), supra, wherein R⁹ represents a halogen atom, or a oxo, C₁-C₃-alkyl-, halo-C₁-C₃-alkyl-, hydroxy-C₁-C₃-alkyl-, —CN, —C(═O)R¹⁰, —C(═O)N(H)R¹⁰, —C(═O)N(R^{10a})R^{10b}, —C(═O)O—R¹⁰, —N(R^{10a})R^{10b}, —NO₂, —N(H)C(═O)R¹⁰, —N(R^{10a})C(═O)R^{10b}, —N(H)C(═O)N(R^{10a})R^{10b}, —N(R^{10a})C(═O)N(R^{10b})R^{10c}, —N(H)S(═O)₂R¹⁰, —N(R^{10a})S(═O)₂R^{10b}, —OR¹⁰, —O(C═O)R¹⁰, —O(C═O)OR¹⁰ or a tetrazolyl- group;

In another preferred embodiment, the invention relates to compounds of formula (I), supra, wherein R⁹ represents a halogen atom, or a oxo, C₁-C₃-alkyl-, halo-C₁-C₃-alkyl-, hydroxy-C₁-C₃-alkyl-, —CN, —C(═O)R¹⁰, —C(═O)N(H)R¹⁰, —C(═O)N(R^{10a})R^{10b}, —C(═O)O—R¹⁰, —N(R^{10a})R^{10b}, —N(R^{10a})C(═O)R^{10b}, —N(R^{10a})C(═O)N(R^{10b})R^{10c}, —N(R^{10a})S(═O)₂R^{10b}, —OR¹⁰, or a tetrazolyl- group;

In another preferred embodiment, the invention relates to compounds of formula (I), supra, wherein R⁹ represents a halogen atom, or a C₁-C₃-alkyl-, hydroxy-C₁-C₃-alkyl-, —CN, —C(═O)N(H)R¹⁰, —C(═O)N(R^{10a})R^{10b}, —C(═O)O—R¹⁰, —N(R^{10a})R^{10b}, —N(R^{10a})C(═O)R^{10b}, —N(R^{10a})C(═O)N(R^{10b})R^{10c}, —OR¹⁰, or a tetrazolyl-group;

In another preferred embodiment, the invention relates to compounds of formula (I), supra, wherein R⁹ represents a halogen atom, or a oxo, C₁-C₃-alkyl-, fluoro-C₁-C₃-alkyl-, hydroxy-C₁-C₃-alkyl-, —CN, —C(═O)R¹⁰, —C(═O)N(H)R¹⁰, —C(═O)N(R^{10a})R^{10b}, —C(═O)O—R¹⁰, —N(R^{10a})R^{10b}, —NO₂, —N(H)C(═O)R¹⁰, —N(R^{10a})C(═O)R^{10b}, —N(H)S(═O)₂R¹⁰, —N(R^{10a})S(═O)₂R^{10b}, —OR¹⁰, —S(═O)₂R¹⁰, —S(═O)₂N(H)R¹⁰, —S(═O)₂N(R^{10a})R^{10b} or a tetrazolyl- group.

In another preferred embodiment, the invention relates to compounds of formula (I), supra, wherein R⁹ represents a halogen atom, or a C₁-C₃-alkyl-, fluoro-C₁-C₃-alkyl-, hydroxy-C₁-C₃-alkyl-, —CN, —C(═O)R¹⁰, —C(═O)N(H)R¹⁰, —C(═O)N(R^{10a})R^{10b}, —N(RIoa)R^{10b}, —N(H)C(═O)R¹⁰, —N(R^{10a})C(═O)R^{10b}, —OR¹⁰ group.

In another preferred embodiment, the invention relates to compounds of formula (I), supra, wherein R⁹ represents a halogen atom, or a C₁-C₃-alkyl-, —CN, —C(═O)NH₂ or —OH group.

In another preferred embodiment, the invention relates to compounds of formula (I), supra, wherein R¹⁰, R^{10a}, R^{10b}, R^{10c} represent, independently from each other, a hydrogen atom or group selected from: C₁-C₃-alkyl-, hydroxy-C₁-C₃-alkyl-.

In another preferred embodiment, the invention relates to compounds of formula (I), supra, wherein R¹⁰, R^{10a}, R^{10b}, R^{10c} represent, independently from each other, a hydrogen atom or a C₁-C₃-alkyl- group.

In another preferred embodiment, the invention relates to compounds of formula (I), supra, wherein R¹⁰, R^{10a}, R^{10b}, R^{10c} represent, independently from each other, a hydrogen atom or a group selected from: C₁-C₃-alkyl-, fluoro-C₁-C₃-alkyl-, hydroxy-C₁-C₃-alkyl-, C₁-C₃-alkoxy-C₁-C₃-alkyl-, C₃-C₇-cycloalkyl-, said C₁-C₃-alkyl- group being optionally substituted once with —N(R¹²)R^{12a}; or wherein R^{10a} and R^{10b}, together with the nitrogen atom they are attached to, represent a 4- to 7-membered heterocycloalkyl- group, said 4- to 7-membered heterocycloalkyl-group being optionally substituted one or more times, identically or differently, with R¹³.

In another preferred embodiment, the invention relates to compounds of formula (I), supra, wherein R¹⁰, R^{10a}, R^{10b}, R^{10c} represent, independently from each other, a hydrogen atom or a group selected from: C₁-C₃-alkyl-, fluoro-C₁-C₃-alkyl-, hydroxy-C₁-C₃-alkyl-, C₁-C₃-alkoxy-C₁-C₃-alkyl-, C₃-C₇-cycloalkyl-, said C₁-C₃-alkyl- group being optionally substituted once with —N(R¹²)R^{12a}.

In another preferred embodiment, the invention relates to compounds of formula (I), supra, wherein R^{10a} and R^{10b}, together with the nitrogen atom they are attached to, represent a 4- to 7-membered heterocycloalkyl- group, said 4- to 7-membered heterocycloalkyl-group being optionally substituted one or more times, identically or differently, with R¹³.

In another preferred embodiment, the invention relates to compounds of formula (I), supra, wherein R¹⁰, R^{10a}, R^{10b}, R^{10c} represent, independently from each other, a hydrogen atom or a group selected from: C₁-C₃-alkyl-, hydroxy-C₁-C₃-alkyl-, C₁-C₃-alkoxy-C₁-C₃-alkyl-;

or wherein R^{10a} and R^{10b}, together with the nitrogen atom they are attached to, represent a 4- to 7-membered heterocycloalkyl- group.

In another preferred embodiment, the invention relates to compounds of formula (I), supra, wherein R¹⁰, R^{10a}, R^{10b}, R^{10c} represent, independently from each other, a hydrogen atom or a group selected from: C₁-C₃-alkyl-, hydroxy-C₁-C₃-alkyl-, C₁-C₃-alkoxy-C₁-C₃-alkyl-.

In another preferred embodiment, the invention relates to compounds of formula (I), supra, wherein R^{10a} and R^{10b}, together with the nitrogen atom they are attached to, represent a 4- to 7-membered heterocycloalkyl- group.

In another preferred embodiment, the invention relates to compounds of formula (I), supra, wherein R¹⁰, R^{10a}, R^{10b}, R^{10c} represent, independently from each other, a hydrogen atom or a C₁-C₃-alkyl- group.

In another preferred embodiment, the invention relates to compounds of formula (I), supra, wherein R¹⁰, R^{10a}, R^{10b}, R^{10c} represent, independently from each other, a hydrogen atom or a methyl- group.

In another preferred embodiment, the invention relates to compounds of formula (I), supra, wherein R¹¹ represents a hydrogen atom or a cyano-, C₁-C₃-alkyl-, —C(═O)R¹⁰, or —C(═O)O—R¹⁰ group.

In another preferred embodiment, the invention relates to compounds of formula (I), supra, wherein R¹¹ represents a hydrogen atom or a cyano-, —C(═O)R¹⁰, or —C(═O)O—R¹⁰ group.

In another preferred embodiment, the invention relates to compounds of formula (I), supra, wherein R¹¹ represents a hydrogen atom or a cyano- or —C(═O)O—R¹⁰ group.

In another preferred embodiment, the invention relates to compounds of formula (I), supra, wherein R¹¹ represents a —C(═O)O—R¹⁰ group.

In another preferred embodiment, the invention relates to compounds of formula (I), supra, wherein R¹¹ represents a cyano- group.

In another preferred embodiment, the invention relates to compounds of formula (I), supra, wherein R¹¹ represents a hydrogen atom.

In another preferred embodiment, the invention relates to compounds of formula (I), supra, wherein R¹² and R^{12a}, independently from each other, represent a hydrogen atom or a C₁-C₃-alkyl- group, or wherein R¹², R^{12a}, together with the nitrogen atom they are attached to, represent a 4- to 7-membered heterocycloalkyl- group.

In another preferred embodiment, the invention relates to compounds of formula (I), supra, wherein R¹² and R^{12a}, independently from each other, represent a hydrogen atom or a C₁-C₃-alkyl- group.

In another preferred embodiment, the invention relates to compounds of formula (I), supra, wherein R¹² and R^{12a}, together with the nitrogen atom they are attached to, represent a 4- to 7-membered heterocycloalkyl- group.

In another preferred embodiment, the invention relates to compounds of formula (I), supra, wherein R¹³ represents a halogen atom or a cyano, hydroxy, oxo, C₁-C₃-alkyl-, trifluoromethyl-, —C(═O)R¹⁰ or —C(═O)O—R¹⁰ group.

In another preferred embodiment, the invention relates to compounds of formula (I), supra, wherein R¹³ represents a fluoro atom or a cyano, hydroxy, oxo, C₁-C₃-alkyl-, trifluoromethyl-, acetyl-, methoxycarbonyl- or ethoxycarbonyl- group.

In another preferred embodiment, the invention relates to compounds of formula (I), supra, wherein L¹ represents a group selected from: —C₁-C₄-alkylene-, —CH₂—CH═CH—, —C(phenyl)(H)—, —CH₂—CH₂—O—, —CH₂—C(═O)—N(H)—, —CH₂—C(═O)—N(R^{10a})—.

In another preferred embodiment, the invention relates to compounds of formula (I), supra, wherein L¹ represents a group selected from:

—C₁-C₄-alkylene-, —C(phenyl)(H)—, —CH₂—CH₂—O—, —CH₂—C(═O)—N(H)—, —CH₂—C(═O)—N(R^{10a})—.

In another preferred embodiment, the invention relates to compounds of formula (I), supra, wherein L¹ represents a group selected from: —C₁-C₄-alkylene-, —CH₂—CH₂—O—.

In another preferred embodiment, the invention relates to compounds of formula (I), supra, wherein L¹ represents a —C₁-C₄-alkylene- group.

In another preferred embodiment, the invention relates to compounds of formula (I), supra, wherein L¹ represents a —C₁-C₃-alkylene- group.

In another preferred embodiment, the invention relates to compounds of formula (I), supra, wherein L¹ represents a group selected from: —CH₂—, —CH₂—CH₂—.

In another preferred embodiment, the invention relates to compounds of formula (I), supra, wherein L¹ represents a group selected from: —CH₂—, —C(CH₃)(H)—, —CH₂—CH₂—.

In another preferred embodiment, the invention relates to compounds of formula (I), supra, wherein L¹ represents a —CH₂— group.

In another preferred embodiment, the invention relates to compounds of formula (I), supra, wherein L¹ represents a group selected from: —CH₂—, —C(CH₃)(H)—.

In another preferred embodiment, the invention relates to compounds of formula (I), supra, wherein L¹ represents a —C(CH₃)(H)— group.

In another preferred embodiment, the invention relates to compounds of formula (I), supra, wherein L² represents a group selected from: —CH₂—, —CH₂—CH₂—.

In another preferred embodiment, the invention relates to compounds of formula (I), supra, wherein L² represents a —CH₂— group.

In another preferred embodiment, the invention relates to compounds of formula (I), supra, wherein L³ represents a —C₁-C₄-alkylene- group.

In another preferred embodiment, the invention relates to compounds of formula (I), supra, wherein L³ represents a —C₁-C₃-alkylene- group.

In another preferred embodiment, the invention relates to compounds of formula (I), supra, wherein L³ represents a —C₁-C₂-alkylene- group.

In another preferred embodiment, the invention relates to compounds of formula (I), supra, wherein p represents an integer of 0 or 1.

In another preferred embodiment, the invention relates to compounds of formula (I), supra, wherein p represents an integer of 0.

In another preferred embodiment, the invention relates to compounds of formula (I), supra, wherein p represents an integer of 1.

In another preferred embodiment, the invention relates to compounds of formula (I), supra, wherein R¹ represents a C₁-C₃-alkyl-, trifluoromethyl- or cyano- group, wherein R² represents a methyl-, ethyl- or trifluoromethyl-group, wherein R^{4b} and R⁶ represent a hydrogen atom, wherein R^{5a}, R^{5c}, R^{5d} independently from each other represent hydrogen atom, a fluoro atom or a chloro atom, and wherein L¹ represents a group selected from —CH₂—, —C(CH₃)(H)—.

It is to be understood that the present invention relates to any sub-combination within any embodiment of compounds of general formula (I), supra.

Some examples of combinations are given hereinafter. However, the invention is not limited to these combinations.

In a preferred embodiment, the present invention relates to compounds of general formula (I):

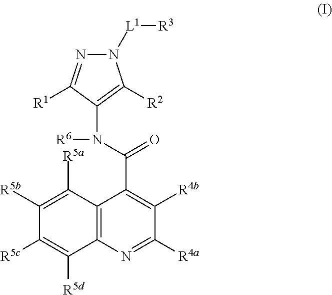

in which:
R¹ represents a C₁-C₃-alkyl-, halo-C₁-C₃-alkyl-, cyano-, —C(═O)O—R¹⁰ or —C(═O)N(R^{10a})R^{10b} group;
R² represents a C₁-C₃-alkyl-, halo-C₁-C₃-alkyl-, cyano-, —C(═O)O—R¹⁰ or —C(═O)N(R^{10a})R^{10b} group;
R³ represents a group selected from: aryl-, heteroaryl-, C₅-C₆-cycloalkyl-, and 5- to 6-membered heterocycloalkyl-;
   wherein said 5- to 6-membered heterocycloalkyl- group is optionally benzocondensed;
   wherein said aryl-, heteroaryl-, C₅-C₆-cycloalkyl-, and 5- to 6-membered heterocycloalkyl- group is substituted, one or more times, identically or differently, with -(L²)ₚ-R⁷;
   and wherein two -(L²)ₚ-R⁷ groups, if being present ortho to each other on an aryl- or heteroaryl- group optionally form a bridge selected from: *—C₃-C₅-alkylene-*, *—O(CH₂)₂O—*, *—O(CH₂)O—*, *—O(CF₂)O—*, *—CH₂C(R^{10a})(R^{10b})O—*, *—C(═O)N(R^{10a})CH₂—*, *—N(R^{10a})C(═O)CH₂O—*, *—NHC(═O)NH—*; wherein each * represents the point of attachment to said aryl- or heteroaryl- group;
R^{4a} represents a hydrogen atom or a halogen atom or a group selected from: cyano-, hydroxy-, C₁-C₃-alkyl-, halo-C₁-C₃-alkyl-, C₁-C₃-alkoxy-, C₃-C₇-cycloalkyl-, 4- to 7-membered heterocycloalkyl-, —C(═O)N(R^{10a})R^{10b}, —N(R^{10a})R^{10b};
R^{4b} represents a hydrogen atom or a group selected from: C₁-C₃-alkoxy-, C₁-C₃-alkyl-, cyano-;
   or
R^{4a} and together R^{4b} form a —C₃-C₅-alkylene- group;
R^{5a}, R^{5b}, R^{5c}, R^{5d}
   independently from each other represent a hydrogen atom, a halogen atom or a group selected from:
   cyano-, —NO₂, C₁-C₃-alkyl-, halo-C₁-C₃-alkyl-, C₁-C₃-alkoxy-, halo-C₁-C₃-alkoxy-, phenyl-, heteroaryl-, —C(═O)R¹⁰, —C(═O)N(H)R¹⁰, —C(═O)N(R^{10a})R^{10b}, —C(═O)O—R¹⁰, —N(R^{10a})R^{10b}, —N(H)C(═O)R¹⁰, —N(R^{10a})C(═O)R^{10b}, —N(H)C(═O)N(R^{10a})R^{10b}, —N(R^{10a})C(═O)N(R^{10b})R^{10c}, —N(R^{10a})C(═O)C(═O)N(R^{10b})R^{10c}, —N(H)C(═O)OR¹⁰, —N(R^{10a})C(═O)OR^{10b}, —N(H)S(═O)₂R¹⁰, —N(R^{10a})S(═O)₂R^{10b}, —OR¹⁰, —O(C═O)R¹⁰, —O(C═O)N(R^{10a})R^{10b}, —O(C═O)OR¹⁰, —SR¹⁰, —S(═O)R¹⁰, —S(═O)₂R¹⁰, —S(═O)₂N(H)R¹⁰, —S(═O)₂N(R^{10a})R^{10b} or —S(═O)(═NR^{10a})R^{10b},
   said phenyl- or heteroaryl- group being optionally substituted one or more times, identically or differently, with a group selected from: halo-, cyano-, C₁-C₃-alkyl-, halo-C₁-C₃-alkyl-, C₁-C₃-alkoxy- group;
R⁶ represents a hydrogen atom or group selected from: C₁-C₃-alkyl-, C₁-C₃-alkoxy-(L²)-, hydroxy-C₁-C₃-alkyl-, aryl-(L²)-, heteroaryl-(L²)-;
R⁷ represents a group selected from: oxo, C₁-C₃-alkyl-, C₃-C₇-cycloalkyl-, 4- to 7-membered heterocycloalkyl-, halo-C₁-C₃-alkyl-, C₁-C₃-alkoxy-, halo-C₁-C₃-alkoxy-, —OH, —CN, halo-, —C(═O)R⁸, —C(═O)—O—R⁸, —C(═O)N(R^{8a})R^{8b}, —S(═O)₂R⁸, —S(═O)(═N)R¹¹, phenyl-, 5- to 6-membered heteroaryl-;
R⁸ represents a hydrogen atom or a C₁-C₆-alkyl-, halo-C₁-C₃-alkyl-, cyano-C₁-C₄-alkyl-, C₁-C₃-alkoxy-C₁-C₃-alkyl-, C₃-C₇-cycloalkyl-, phenyl-, 5- to 6-membered heteroaryl- or benzyl- group;
R^{8a}, R^{8b}
   represent, independently from each other, a hydrogen atom, or a C₁-C₁₀-alkyl-, C₃-C₇-cycloalkyl-, (C₃-C₇-cycloalkyl)-(L³)-, C₃-C₆-alkenyl-, C₃-C₆-alkynyl-, 4- to 10-membered heterocycloalkyl-,
   (4- to 10-membered heterocycloalkyl)-(L³)-, phenyl-, heteroaryl-, phenyl-(L³)-, (phenyl)-O-(L³)-, heteroaryl-(L³)-, or
   (aryl)-(4- to 10-membered heterocycloalkyl)- group;
   said C₁-C₁₀-alkyl-, C₃-C₇-cycloalkyl-, (C₃-C₇-cycloalkyl)-(L³)-, C₃-C₆-alkenyl-, C₃-C₆-alkynyl-, 4- to 10-membered heterocycloalkyl-, (4- to 10-membered heterocycloalkyl)-(L³)-, phenyl-, heteroaryl-, phenyl-(L³)-, (phenyl)-O-(L³)-, heteroaryl-(L³)-, and (aryl)-(4- to 10-membered heterocycloalkyl)- group being optionally substituted one or more times, identically or differently, with R⁹;
      or
R^{8a} and R^{8b}, together with the nitrogen atom they are attached to, represent a 4- to 10-membered heterocycloalkyl-group, said 4- to 10-membered heterocycloalkyl-group being optionally substituted one or more times, identically or differently, with R⁹;
R⁹ represents a halogen atom, or a oxo, C₁-C₃-alkyl-, halo-C₁-C₃-alkyl-, hydroxy-C₁-C₃-alkyl-, —CN, —C(═O)R¹⁰, —C(═O)N(H)R¹⁰, —C(═O)N(R^{10a})R^{10b}, —C(═O)O—R¹⁰, —N(R^{10a})R^{10b}, —NO₂, —N(H)C(═O)R¹⁰, —N(R^{10a})C(═O)R^{10b}, —N(H)C(═O)N(R^{10a})R^{10b}, —N(R^{10a})C(═O)N(R^{10b})R^{10c}, —N(H)C(═O)OR¹⁰, —N(R^{10a})C(═O)OR^{10b}, —N(H)S(═O)₂R¹⁰, —N(R^{10a})S(═O)₂R^{10b}, —OR¹⁰, —O(C═O)R¹⁰, —O(C═O)N(R^{10a})Rob, —O(C═O)OR¹⁰, —SR¹⁰, —S(═O)R¹⁰, —S(═O)₂R¹⁰, —S(═O)₂N(H)R¹⁰, —S(═O)₂N(R^{10a})R^{10b}, —S(═O)(═NR^{10a})R^{10b} or a tetrazolyl-group;
   or
two R⁹ groups present ortho to each other on a phenyl- or heteroaryl-ring form a bridge selected from: *—C₃-C₅-alkylene-*, *—O(CH₂)₂O—*, *—O(CH₂)O—*, *—O(CF₂)O—*, *—CH₂C(R^{10a})(R^{10b})O—*, *—C(═O)N(R^{10a})CH₂—*, *—N(R^{10a})C(═O)CH₂O—*, *—NHC(═O)NH—*; wherein each * represents the point of attachment to said phenyl- or heteroaryl- ring;
R¹⁰, R^{10a}, R^{10b}, R^{10c}
   represent, independently from each other, a hydrogen atom or a group selected from: C₁-C₃-alkyl-, halo-C₁-C₃-alkyl-, hydroxy-C₁-C₃-alkyl-, C₃-C₇-cycloalkyl-;
R¹¹ represents a hydrogen atom or a cyano-, C₁-C₃-alkyl-, —C(═O)R¹⁰, —C(═O)N(H)R¹⁰, —C(═O)N(R^{10a})R^{10b} or —C(═O)O—R¹⁰ group;
L¹ represents a group selected from: —C₁-C₄-alkylene-, —CH₂—CH═CH—, —C(phenyl)(H)—, —CH₂—CH₂—O—;
L² represents a group selected from: —CH₂—, —CH₂—CH₂—, —CH₂—CH₂—CH₂—;
L³ represents a —C₁-C₆-alkylene- group;
p is an integer of 0 or 1;
   or a tautomer, a stereoisomer, an N-oxide, a hydrate, a solvate, or a salt thereof, or a mixture of same.

In a preferred embodiment, the present invention relates to compounds of general formula (I):

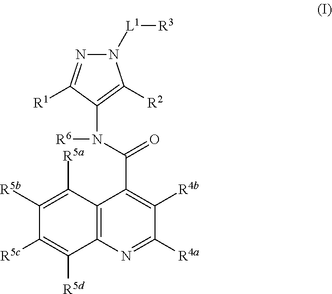

in which:
R¹ represents a C₁-C₃-alkyl-, halo-C₁-C₃-alkyl-, cyano-, —C(═O)O—R¹⁰ or —C(═O)N(R^{10a})R^{10b} group;
R² represents a C₁-C₃-alkyl-, halo-C₁-C₃-alkyl-, cyano-, —C(═O)O—R¹⁰ or —C(═O)N(R^{10a})R^{10b} group;
R³ represents a group selected from: aryl-, heteroaryl-, C₅-C₆-cycloalkyl-, and 5- to 6-membered heterocycloalkyl-;
   wherein said 5- to 6-membered heterocycloalkyl- group is optionally benzocondensed;
   wherein said aryl-, heteroaryl-, C₅-C₆-cycloalkyl-, and 5- to 6-membered heterocycloalkyl- group is substituted, one or more times, identically or differently, with -(L²)ₚ-R;
   and wherein two -(L²)ₚ-R⁷ groups, if being present ortho to each other on an aryl- or heteroaryl- group optionally form a bridge selected from: *—C₃-C₅-alkylene-*, *—O(CH₂)₂O—*, *—O(CH₂)O—*, *—O(CF₂)O—*,
   wherein each * represents the point of attachment to said aryl- or heteroaryl- group;
R^{4a} represents a hydrogen atom or a halogen atom or a group selected from: cyano-, hydroxy-, C₁-C₃-alkyl-, halo-C₁-C₃-alkyl-, C₁-C₃-alkoxy-, C₃-C₇-cycloalkyl-, 4- to 7-membered heterocycloalkyl-, —C(═O)N(R^{10a})R^{10b}, —N(R^{10a})R^{10b};
R^{4b} represents a hydrogen atom or a group selected from: C₁-C₃-alkoxy-, C₁-C₃-alkyl-, cyano-;
   or
R^{4a} and together R^{4b} form a —C₃-C₅-alkylene- group;
R^{5a}, R^{5b}, R^{5c}, R^{5d}
   independently from each other represent a hydrogen atom, a halogen atom or a group selected from:
   cyano-, —NO₂, C₁-C₃-alkyl-, halo-C₁-C₃-alkyl-, C₁-C₃-alkoxy-, halo-C₁-C₃-alkoxy-, phenyl-, heteroaryl-, —C(═O)R¹⁰, —C(═O)N(H)R¹⁰, —C(═O)N(R^{10a})R^{10b}, —C(═O)O—R¹⁰, —N(R^{10a})R^{10b}, —N(H)C(═O)R¹⁰, —N(R^{10a})C(═O)R^{10b}, —N(R^{10a})C(═O)C(═O)N(R^{10b})R^{10c}, —N(H)S(═O)₂R¹⁰, said phenyl- or heteroaryl- group being optionally substituted one or more times, identically or differently, with a C₁-C₃-alkyl- group;
R⁶ represents a hydrogen atom or group selected from: C₁-C₃-alkyl-, C₁-C₃-alkoxy-(L²)-, aryl-(L²)-;
R⁷ represents a group selected from: oxo, C₁-C₃-alkyl-, halo-C₁-C₃-alkyl-, C₁-C₃-alkoxy-, halo-C₁-C₃-alkoxy-, —OH, —CN, halo-, —C(═O)R⁸, —C(═O)—O—R⁸, —C(═O)N(R^{8a})R^{8b}, —S(═O)₂R⁸, phenyl;
R⁸ represents a hydrogen atom or a C₁-C₆-alkyl-, C₃-C₇-cycloalkyl-, phenyl- or benzyl- group;
R^{8a}, R^{8b}
   represent, independently from each other, a hydrogen atom, or a C₁-C₁₀-alkyl-, C₃-C₇-cycloalkyl-, (C₃-C₇-cycloalkyl)-(L³)-, C₃-C₆-alkenyl-, C₃-C₆-alkynyl-, 4- to 10-membered heterocycloalkyl-,
   (4- to 10-membered heterocycloalkyl)-(L³)-, phenyl-, heteroaryl-, phenyl-(L³)-, or (phenyl)-O-(L³)-, heteroaryl-(L³)- group;
   said C₁-C₁₀-alkyl-, C₃-C₇-cycloalkyl-, (C₃-C₇-cycloalkyl)-(L³)-, C₃-C₆-alkenyl-, C₃-C₆-alkynyl-, 4- to 10-membered heterocycloalkyl-, (4- to 10-membered heterocycloalkyl)-(L³)-, phenyl-, heteroaryl-, phenyl-(L³)-, (phenyl)-O-(L³)-, and heteroaryl-(L³)- group being optionally substituted one or more times, identically or differently, with R⁹;
      or
R^{8a} and R^{8b}, together with the nitrogen atom they are attached to,
   represent a 4- to 10-membered heterocycloalkyl-group, said 4- to 10-membered heterocycloalkyl-group being optionally substituted one or more times, identically or differently, with R⁹;
R⁹ represents a halogen atom, or a oxo, C₁-C₃-alkyl-, halo-C₁-C₃-alkyl-, hydroxy-C₁-C₃-alkyl-, —CN, —C(═O)R¹⁰, —C(═O)N(H)R¹⁰, —C(═O)N(R^{10a})R^{10b}, —C(═O)O—R¹⁰, —N(R^{10a})R^{10b}, —NO₂, —N(H)C(═O)R¹⁰, —N(R^{10a})C(═O)R^{10b}, —N(H)C(═O)N(R^{10a})R^{10b}, —N(R^{10a})C(═O)N(R^{10b})R^{10c}, —N(H)S(═O)₂R¹⁰, —N(R^{10a})S(═O)₂R^{10b}, —OR¹⁰, —O(C═O)R¹⁰, —O(C═O)OR¹⁰ or a tetrazolyl- group;
R¹⁰, R^{10a}, R^{10b}, R^{10c}
   represent, independently from each other, a hydrogen atom or a group selected from: C₁-C₃-alkyl-, hydroxy-C₁-C₃-alkyl-, C₃-C₇-cycloalkyl-;
R¹¹ represents a hydrogen atom or a cyano-, —C(═O)R¹⁰, or —C(═O)O—R¹⁰ group;
L¹ represents a group selected from: —C₁-C₄-alkylene-, —CH₂—CH═CH—, —C(phenyl)(H)—, —CH₂—CH₂—O—;
L² represents a group selected from: —CH₂—, —CH₂—CH₂—.
L³ represents a —C₁-C₆-alkylene- group;
p is an integer of 0 or 1;
   or a tautomer, a stereoisomer, an N-oxide, a hydrate, a solvate, or a salt thereof, or a mixture of same.

In another preferred embodiment, the present invention relates to compounds of general formula (I):

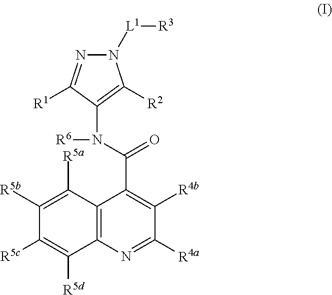

in which:
R¹ represents a C₁-C₃-alkyl-, fluoro-C₁-C₃-alkyl-, cyano-, —C(═O)O—R¹⁰ or —C(═O)N(R^{10a})R^{10b} group;
R² represents a C₁-C₃-alkyl-, fluoro-C₁-C₃-alkyl-, —C(═O)O—R¹⁰ or —C(═O)N(R^{10a})R^{10b} group;
R³ represents a group selected from: aryl-, heteroaryl-, C₅-C₆-cycloalkyl-, and 5- to 6-membered heterocycloalkyl-;
   wherein said 5- to 6-membered heterocycloalkyl- group is optionally benzocondensed;
   wherein said aryl-, heteroaryl-, C₅-C₆-cycloalkyl-, and 5- to 6-membered heterocycloalkyl- group is substituted, one or more times, identically or differently, with -(L²)ₚ-R⁷;
   and wherein two -(L²)ₚ-R⁷ groups, if being present ortho to each other on an aryl- or heteroaryl- group optionally form a *—C₃-C₅-alkylene-* bridge, wherein each * represents the point of attachment to said aryl- or heteroaryl- group;
R^{4a} represents a hydrogen atom or a halogen atom or a group selected from: cyano-, hydroxy-, C₁-C₃-alkyl-, fluoro-C₁-C₃-alkyl-, C₁-C₃-alkoxy-, C₃-C₇-cycloalkyl-, 4- to 7-membered heterocycloalkyl-, —C(═O)N(R^{10a})R^{10b}, —N(R^{10a})R^{10b};
R^{4b} represents a hydrogen atom or a group selected from: C₁-C₃-alkoxy-, C₁-C₃-alkyl-, cyano-;
   or
R^{4a} and together R^{4b} form a —C₃-C₅-alkylene- group;
R^{5a}, R^{5b}, R^{5c}, R^{5d}
   independently from each other represent a hydrogen atom, a halogen atom or a group selected from:
   cyano-, —NO₂, C₁-C₃-alkyl-, fluoro-C₁-C₃-alkyl-, C₁-C₃-alkoxy-, fluoro-C₁-C₃-alkoxy-, phenyl-, heteroaryl-, —C(═O)N(R^{10a})R^{10b}, —C(═O)O—R¹⁰, —N(R^{10a})R^{10b}, —N(H)C(═O)R¹⁰, —N(R^{10a})C(═O)R^{10b}, —N(R^{10a})C(═O)C(═O)N(R^{10b})R^{10c}, —N(H)S(═O)₂R¹⁰, said phenyl- or heteroaryl- group being optionally substituted one or more times, identically or differently, with a C₁-C₃-alkyl- group;
R⁶ represents a hydrogen atom or group selected from: C₁-C₃-alkyl-, C₁-C₃-alkoxy-(L²)-, aryl-(L²)-;
R⁷ represents a group selected from: oxo, C₁-C₃-alkyl-, halo-C₁-C₃-alkyl-, C₁-C₃-alkoxy-, halo-C₁-C₃-alkoxy-, —OH, —CN, halo-, —C(═O)R⁸, —C(═O)—O—R⁸, —C(═O)N(R^{8a})R^{8b}, —S(═O)₂R⁸, phenyl;
R⁸ represents a hydrogen atom or a C₁-C₆-alkyl- group;
R^{8a}, R^{8b}
   represent, independently from each other, a hydrogen atom, or a C₁-C₁₀-alkyl-, C₃-C₇-cycloalkyl-, (C₃-C₇-cycloalkyl)-(L³)-, C₃-C₆-alkenyl-, C₃-C₆-alkynyl-, 4- to 10-membered heterocycloalkyl-,
   (4- to 10-membered heterocycloalkyl)-(L³)-, phenyl-, heteroaryl-, phenyl-(L³)-, or (phenyl)-O-(L³)-, heteroaryl-(L³)- group;
   said C₁-C₁₀-alkyl-, C₃-C₇-cycloalkyl-, (C₃-C₇-cycloalkyl)-(L³)-, C₃-C₆-alkenyl-, C₃-C₆-alkynyl-, 4- to 10-membered heterocycloalkyl-, (4- to 10-membered heterocycloalkyl)-(L³)-, phenyl-, heteroaryl-, phenyl-(L³)-, (phenyl)-O-(L³)-, and heteroaryl-(L³)- group being optionally substituted one or more times, identically or differently, with R⁹;
      or
R^{8a} and R^{8b}, together with the nitrogen atom they are attached to, represent a 4- to 10-membered heterocycloalkyl-group, said 4- to 10-membered heterocycloalkyl-group being optionally substituted one or more times, identically or differently, with R⁹;
R⁹ represents a halogen atom, or a oxo, C₁-C₃-alkyl-, hydroxy-C₁-C₃-alkyl-, —CN, —C(═O)N(H)R¹⁰, —C(═O)N(R^{10a})R^{10b}, —C(═O)O—R¹⁰, —N(R^{10a})R^{10b}, —N(H)C(═O)R¹⁰, —N(R^{10a})C(═O)R^{10b}, —N(R^{10a})C(═O)N(R^{10b})R^{10c}, —OR¹⁰, or a tetrazolyl- group;
R¹⁰, R^{10a}, R^{10b}, R^{10c}
   represent, independently from each other, a hydrogen atom or a group selected from: C₁-C₃-alkyl-, hydroxy-C₁-C₃-alkyl-, C₃-C₇-cycloalkyl-;
R¹¹ represents a hydrogen atom or a cyano- or —C(═O)O—R¹⁰ group;
L¹ represents a group selected from: —C₁-C₃-alkylene-, —CH₂—CH═CH—, —C(phenyl)(H)—, —CH₂—CH₂—O—;
L² represents a group selected from: —CH₂—, —CH₂—CH₂—.
L³ represents a —C₁-C₆-alkylene- group;
p is an integer of 0 or 1;
   or a tautomer, a stereoisomer, an N-oxide, a hydrate, a solvate, or a salt thereof, or a mixture of same.

In another preferred embodiment, the present invention relates to compounds of general formula (I):

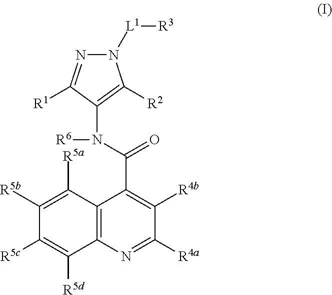

in which:
R¹ represents a methyl- or trifluoromethyl- group; and
R² represents a methyl- group;
   or
R¹ represents a methyl-; and
R² represents a methyl- or trifluoromethyl- group;
R³ represents a group selected from: aryl-, heteroaryl-, C₅-C₆-cycloalkyl-, and 5- to 6-membered heterocycloalkyl-;
   wherein said 5- to 6-membered heterocycloalkyl- group is optionally benzocondensed;
   wherein said aryl-, heteroaryl-, C₅-C₆-cycloalkyl-, and 5- to 6-membered heterocycloalkyl- group is substituted, one or more times, identically or differently, with -(L²)ₚ-R⁷;
   and wherein two -(L²)ₚ-R⁷ groups, if being present ortho to each other on an aryl- or heteroaryl- group optionally form a bridge selected from: *—C₃-C₅-alkylene-*; wherein each * represents the point of attachment to said aryl- or heteroaryl- group;
R^{4a} represents a hydrogen atom or a halogen atom or a group selected from: cyano-, hydroxy-, C₁-C₃-alkyl-, halo-C₁-C₃-alkyl-, C₁-C₃-alkoxy-, C₃-C₇-cycloalkyl-, 4- to 7-membered heterocycloalkyl-, —C(═O)N(R^{10a})R^{10b}, —N(R^{10a})R^{10b};
R^{4b} represents a hydrogen atom or a group selected from: C₁-C₃-alkoxy-, C₁-C₃-alkyl-, cyano-;
   or
R^{4a} and together R^{4b} form a —C₃-C₅-alkylene- group;
R^{5a}, R^{5b}, R^{5c}, R^{5d}
   independently from each other represent a hydrogen atom, a halogen atom or a group selected from:
   cyano-, —NO₂, C₁-C₃-alkyl-, halo-C₁-C₃-alkyl-, C₁-C₃-alkoxy-, halo-C₁-C₃-alkoxy-, phenyl-, heteroaryl-, —C(═O)R¹⁰, —C(═O)N(H)R¹⁰, —C(═O)N(R^{10a})R^{10b}, —C(═O)O—R¹⁰, —N(R^{10a})R^{10b}, —N(H)C(═O)R¹⁰, —N(R^{10a})C(═O)R^{10b}, —N(H)C(═O)N(R^{10a})R^{10b}, —N(R^{10a})C(═O)N(R^{10b})R^{10c}, —N(R^{10a})C(═O)C(═O)N(R^{10b})R^{10c}, —N(H)C(═O)OR¹⁰, —N(R^{10a})C(═O)OR^{10b}, —N(H)S(═O)₂R¹⁰, —N(R^{10a})S(═O)₂R^{10b}, —OR¹⁰, —O(C═O)R¹⁰, —O(C═O)N(R^{10a})R^{10b}, —O(C═O)OR¹⁰, —SR¹⁰, —S(═O)R¹⁰, —S(═O)₂R¹⁰, —S(═O)₂N(H)R¹⁰, —S(═O)₂N(R^{10a})R^{10b} or —S(═O)(═NR^{10a})R^{10b},
   said phenyl- or heteroaryl- group being optionally substituted one or more times, identically or differently, with a group selected from: halo-, cyano-, C₁-C₃-alkyl-, halo-C₁-C₃-alkyl-, C₁-C₃-alkoxy- group;
R⁶ represents a hydrogen atom or group selected from: C₁-C₃-alkyl-, C₁-C₃-alkoxy-(L²)-, hydroxy-C₁-C₃-alkyl-, aryl-(L²)-, heteroaryl-(L²)-;
R⁷ represents a group selected from: oxo, C₁-C₃-alkyl-, C₃-C₇-cycloalkyl-, 4- to 7-membered heterocycloalkyl-, halo-C₁-C₃-alkyl-, C₁-C₃-alkoxy-, halo-C₁-C₃-alkoxy-, —OH, —CN, halo-, —C(═O)R⁸, —C(═O)—O—R⁸, —C(═O)N(R^{8a})R^{8b}, —S(═O)₂R⁸, phenyl-, 5- to 6-membered heteroaryl-;
R⁸ represents a hydrogen atom or a C₁-C₆-alkyl-, halo-C₁-C₃-alkyl-, cyano-C₁-C₄-alkyl-, C₁-C₃-alkoxy-C₁-C₃-alkyl-, C₃-C₇-cycloalkyl-, phenyl-, 5- to 6-membered heteroaryl- or benzyl- group; R^{8a}, R^{8b}
   represent, independently from each other, a hydrogen atom, or a C₁-C₁₀-alkyl-, C₃-C₇-cycloalkyl-, (C₃-C₇-cycloalkyl)-(L³)-, C₃-C₆-alkenyl-, C₃-C₆-alkynyl-, 4- to 10-membered heterocycloalkyl-,
   (4- to 10-membered heterocycloalkyl)-(L³)-, phenyl-, heteroaryl-, phenyl-(L³)-, (phenyl)-O-(L³)-, heteroaryl-(L³)-, or
   (aryl)-(4- to 10-membered heterocycloalkyl)- group;
   said C₁-C₁₀-alkyl-, C₃-C₇-cycloalkyl-, (C₃-C₇-cycloalkyl)-(L³)-, C₃-C₆-alkenyl-, C₃-C₆-alkynyl-, 4- to 10-membered heterocycloalkyl-, (4- to 10-membered heterocycloalkyl)-(L³)-, phenyl-, heteroaryl-, phenyl-(L³)-, (phenyl)-O-(L³)-, heteroaryl-(L³)-, and (aryl)-(4- to 10-membered heterocycloalkyl)- group being optionally substituted one or more times, identically or differently, with R⁹;
      or
R^{8a} and R^{8b}, together with the nitrogen atom they are attached to,
   represent a 4- to 10-membered heterocycloalkyl-group, said 4- to 10-membered heterocycloalkyl-group being optionally substituted one or more times, identically or differently, with R⁹;
R⁹ represents a halogen atom, or a oxo, C₁-C₃-alkyl-, halo-C₁-C₃-alkyl-, hydroxy-C₁-C₃-alkyl-, —CN, —C(═O)R¹⁰, —C(═O)N(H)R¹⁰, —C(═O)N(R^{10a})R^{10b}, —C(═O)O—R¹⁰, —N(R^{10a})R^{10b}, —NO₂, —N(H)C(═O)R¹⁰, —N(R^{10a})C(═O)R^{10b}, —N(H)C(═O)N(R^{10a})R^{10b}, —N(R^{10a})C(═O)N(R^{10b})R^{10c}, —N(H)C(═O)OR¹⁰, —N(R^{10a})C(═O)OR^{10b}, —N(H)S(═O)₂R¹⁰, —N(R^{10a})S(═O)₂R^{10b}, —OR¹⁰, —O(C═O)R¹⁰, —O(C═O)N(R^{10a})R^{10b}, —O(C═O)OR¹⁰, —SR¹⁰, —S(═O)R¹⁰, —S(═O)₂R¹⁰, —S(═O)₂N(H)R¹⁰, —S(═O)₂N(R^{10a})R^{10b}, —S(═O)(═NR^{10a})R^{10b} or a tetrazolyl-group;
R¹⁰, R^{10a}, R^{10b}, R^{10c}
   represent, independently from each other, a hydrogen atom or group selected from: C₁-C₃-alkyl-, halo-C₁-C₃-alkyl-, hydroxy-C₁-C₃-alkyl-, C₃-C₇-cycloalkyl-;
L¹ represents a group selected from: —C₁-C₄-alkylene-, —CH₂—CH═CH—, —C(phenyl)(H)—, —CH₂—CH₂—O—;
L² represents a group selected from: —CH₂—, —CH₂—CH₂—, —CH₂—CH₂—CH₂—;
L³ represents a —C₁-C₆-alkylene- group;
p is an integer of 0 or 1;
   or a tautomer, a stereoisomer, an N-oxide, a hydrate, a solvate, or a salt thereof, or a mixture of same.

In another preferred embodiment, the present invention relates to compounds of general formula (I):

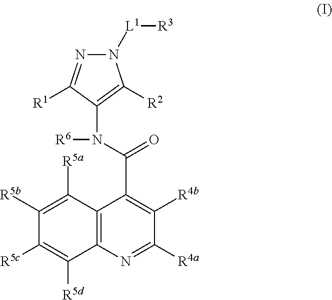

in which:
R¹ represents a C₁-C₃-alkyl-, fluoro-C₁-C₃-alkyl-, cyano-, —C(═O)O—R¹⁰ or —C(═O)N(R^{10a})R^{10b} group;
R² represents a C₁-C₃-alkyl-, fluoro-C₁-C₃-alkyl-, —C(═O)O—R¹⁰ or —C(═O)N(R^{10a})R^{10b} group;
R³ represents a group selected from: aryl-, heteroaryl-, C₅-C₆-cycloalkyl-, and 5- to 6-membered heterocycloalkyl-;
   wherein said 5- to 6-membered heterocycloalkyl- group is optionally benzocondensed;
   wherein said aryl-, heteroaryl-, C₅-C₆-cycloalkyl-, and 5- to 6-membered heterocycloalkyl- group is optionally substituted, one or more times, identically or differently, with -(L²)ₚ-R⁷;
   and wherein two -(L²)ₚ-R⁷ groups, if being present ortho to each other on an aryl- or heteroaryl- group optionally form a bridge selected from: *—C₃-C₈-alkylene-*, *—O(CH₂)₂O—*, *—O(CH₂)O—; wherein each * represents the point of attachment to said aryl- or heteroaryl- group;
R^{4a} represents a hydrogen atom or a halogen atom or a group selected from: cyano-, hydroxy-, C₁-C₃-alkyl-, fluoro-C₁-C₃-alkyl-, C₁-C₃-alkoxy-, fluoro-C₁-C₃-alkoxy-, C₃-C₇-cycloalkyl-, 4- to 7-membered heterocycloalkyl-, —C(═O)—OR¹⁰, —C(═O)N(R^{10a})R^{10b},
   —C(═O)—N(R^{10a})—S(═O)₂—R¹⁰, —SR¹⁰, —S(═O)—R¹⁰, —S(═NR¹¹)—R¹⁰, —S(═O)₂—R¹⁰, —S(═O)₂—N(R^{10a})R^{10b}, —S(═O)(═NR^{10a})—R¹⁰, —N(R^{10a})R^{10b};
R^{4b} represents a hydrogen atom or a group selected from: C₁-C₃-alkoxy-, C₁-C₃-alkyl-, cyano-;
   or
R^{4a} and together R^{4b} form a —C₃-C₅-alkylene- group;
R^{5a}, R^{5b}, R^{5c}, R^{5d}
   independently from each other represent a hydrogen atom, a halogen atom or a group selected from:
   cyano-, —NO₂, C₁-C₃-alkyl-, fluoro-C₁-C₃-alkyl-, C₁-C₃-alkoxy-, fluoro-C₁-C₃-alkoxy-, phenyl-, heteroaryl-, —C(═O)R¹⁰, —C(═O)N(H)R¹⁰, —C(═O)N(R^{10a})R^{10b}, —C(═O)O—R¹⁰, —N(R^{10a})R^{10b}, —N(H)C(═O)R¹⁰, —N(R^{10a})C(═O)R^{10b}, —N(H)S(═O)₂R¹⁰, —N(R^{10a})S(═O)₂R^{10b}, —OR¹⁰, —S(═O)₂R¹⁰, —S(═O)₂N(H)R¹⁰ or —S(═O)₂N(R^{10a})R^{10b},
   said phenyl- or heteroaryl- group being optionally substituted one or more times, identically or differently, with a group selected from: halo-, cyano-, C₁-C₃-alkyl-, fluoro-C₁-C₃-alkyl-, C₁-C₃-alkoxy-;
R⁶ represents a hydrogen atom or group selected from: C₁-C₃-alkyl-, C₁-C₃-alkoxy-(L²)-, hydroxy-C₁-C₃-alkyl-, aryl-(L²)-, heteroaryl-(L²)-;
R⁷ represents a group selected from: oxo, C₁-C₄-alkyl-, C₃-C₇-cycloalkyl-, 4- to 7-membered heterocycloalkyl-, fluoro-C, —C₄-alkyl-, hydroxy-C₁-C₄-alkyl-, cyano-C₁-C₄-alkyl-, C₂-C₄-alkenyl-, C₁-C₄-alkoxy-, fluoro-C₁-C₄-alkoxy-, —OH, —CN, halo-, —C(═O)R⁸, —C(═O)—O—R⁸, —C(═O)N(R^{8a})R^{8b}, —N(R^{10a})R^{10b}, —S(═O)₂R⁸, —S(═O)(═NR¹¹)—R¹⁰, phenyl-, 5- to 6-membered heteroaryl-;
R⁸ represents a hydrogen atom or a C₁-C₆-alkyl-, fluoro-C₁-C₃-alkyl-, cyano-C₁-C₄-alkyl-, C₁-C₃-alkoxy-C₁-C₃-alkyl-, C₃-C₇-cycloalkyl-, phenyl-, 5- to 6-membered heteroaryl- or benzyl- group;
R^{8a}, R^{8b}
   represent, independently from each other, a hydrogen atom, or a C₁-C₆-alkyl-, C₃-C₇-cycloalkyl-, (C₃-C₇-cycloalkyl)-(L³)-, 4- to 10-membered heterocycloalkyl-,
   (4- to 10-membered heterocycloalkyl)-(L³)-, phenyl-, heteroaryl-, phenyl-(L³)-, (phenyl)-O-(L³)-, heteroaryl-(L³)-, or (aryl)-(4- to 10-membered heterocycloalkyl)- group;
   said C₁-C₆-alkyl-, C₃-C₇-cycloalkyl-, (C₃-C₇-cycloalkyl)-(L³)-, 4- to 10-membered heterocycloalkyl-, (4- to 10-membered heterocycloalkyl)-(L³)-, phenyl-, heteroaryl-, phenyl-(L³)-, (phenyl)-O-(L³)-, heteroaryl-(L³)-, and (aryl)-(4- to 10-membered heterocycloalkyl)- group being optionally substituted one or more times, identically or differently, with R⁹;
      or
R^{8a} and R^{8b}, together with the nitrogen atom they are attached to,
   represent a 4- to 10-membered heterocycloalkyl-group, said 4- to 10-membered heterocycloalkyl- group being optionally substituted one or more times, identically or differently, with R⁹;
R⁹ represents a halogen atom, or a oxo, C₁-C₃-alkyl-, fluoro-C₁-C₃-alkyl-, hydroxy-C₁-C₃-alkyl-, —CN, —C(═O)R¹⁰, —C(═O)N(H)R¹⁰, —C(═O)N(R^{10a})R^{10b}, —C(═O)O—R¹⁰, —N(R^{10a})R^{10b}, —NO₂, —N(H)C(═O)R¹⁰, —N(R^{10a})C(═O)R^{10b}, —N(H)S(═O)₂R¹⁰, —N(R^{10a})S(═O)₂R^{10b}, —OR¹⁰, —S(═O)₂R¹⁰, —S(═O)₂N(H)R¹⁰, —S(═O)₂N(R^{10a})R^{10b} or a tetrazolyl- group;
R¹⁰, R^{10a}, R^{10b}, R^{10c}
   represent, independently from each other, a hydrogen atom or a group selected from: C₁-C₃-alkyl-, fluoro-C₁-C₃-alkyl-, hydroxy-C₁-C₃-alkyl-, C₁-C₃-alkoxy-C₁-C₃-alkyl-, C₃-C₇-cycloalkyl-, said C₁-C₃-alkyl- group being optionally substituted once with —N(R¹²)R^{12a};
      or
R^{10a} and R^{10b}, together with the nitrogen atom they are attached to,
   represent a 4- to 7-membered heterocycloalkyl- group, said 4- to 7-membered heterocycloalkyl-group being optionally substituted one or more times, identically or differently, with R¹³;
R¹¹ represents a hydrogen atom or a cyano-, C₁-C₃-alkyl-, —C(═O)R¹⁰ or —C(═O)O—R¹⁰ group;
R¹², R^{12a}
   represent, independently from each other, a hydrogen atom or a C₁-C₃-alkyl- group,
   or
R¹², R^{12a},
   together with the nitrogen atom they are attached to, represent a 4- to 7-membered heterocycloalkyl- group;
R¹³ represents a halogen atom or a cyano, hydroxy, oxo, C₁-C₃-alkyl-, trifluoromethyl-, —C(═O)R¹⁰ or —C(═O)O—R¹⁰ group;
L¹ represents a group selected from: —C₁-C₄-alkylene-, —CH₂—CH═CH—, —C(phenyl)(H)—, —CH₂—CH₂—O—, —CH₂—C(═O)—N(H)—, —CH₂—C(═O)—N(R^{10a})—;
L² represents a group selected from: —CH₂—, —CH₂—CH₂—, —CH₂—CH₂—CH₂—;
L³ represents a —C₁-C₄-alkylene- group;
p is an integer of 0 or 1;
   with the proviso that at least one of R¹ and R² is different from iso-propyl-,
   or a tautomer, a stereoisomer, an N-oxide, a hydrate, a solvate, or a salt thereof, or a mixture of same.

In another preferred embodiment, the present invention relates to compounds of general formula (I):

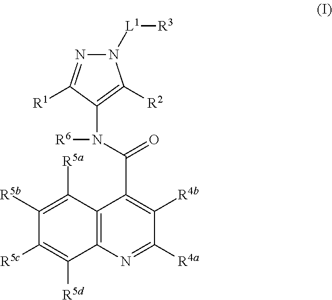

in which:
R¹ represents a C₁-C₃-alkyl-, fluoro-C₁-C₃-alkyl-, cyano-, —C(═O)O—R¹⁰ or —C(═O)N(R^{10a})R^{10b} group;
R² represents a C₁-C₃-alkyl-, fluoro-C₁-C₃-alkyl- or —C(═O)N(R^{10a})R^{10b} group;
   R³ represents a group selected from: aryl- or 5- to 6-membered heteroaryl- or piperidinyl-;
   wherein said aryl- or 5- to 6-membered heteroaryl- or piperidinyl- group is optionally substituted, one or more times, identically or differently, with -(L²)ₚ-R⁷;
R^{4a} represents a hydrogen atom or a halogen atom or a group selected from: cyano-, hydroxy-, C₁-C₃-alkyl-, fluoro-C₁-C₃-alkyl-, C₁-C₃-alkoxy-, fluoro-C₁-C₃-alkoxy-, C₃-C₇-cycloalkyl-, 4- to 7-membered heterocycloalkyl-, —C(═O)—OR¹⁰, —C(═O)N(R^{10a})R^{10b}, —C(═O)—N(R^{10a})—S(═O)₂—R¹⁰, —SR¹⁰, —S(═O)—R¹⁰, —S(═NR¹¹)—R¹, —S(═O)₂—R¹⁰, —S(═O)₂—N(R^{10a})R^{10b}, —S(═O)(═NR¹¹)_R¹¹)—R¹⁰, —N(R^{10a})R^{10b};
R^{4b} represents a hydrogen atom or a group selected from: C₁-C₃-alkoxy-, C₁-C₃-alkyl-, cyano-;
   or
R^{4a} and together R^{4b} form a —C₃-C₅-alkylene- group;
R^{5a}, R^{5b}, R^{5c}, R^{5d}
   independently from each other represent a hydrogen atom, a halogen atom or a group selected from:
   cyano-, —NO₂, C₁-C₃-alkyl-, fluoro-C₁-C₃-alkyl-, C₁-C₃-alkoxy-, fluoro-C₁-C₃-alkoxy-, phenyl-, heteroaryl-, —C(═O)R¹⁰, —C(═O)N(H)R¹⁰, —C(═O)N(R^{10a})R^{10b}, —C(═O)O—R¹⁰, —N(R^{10a})R^{10b}, —N(H)C(═O)R¹⁰, —N(R^{10a})C(═O)R^{10b}, —N(H)S(═O)₂R¹⁰, —N(R^{10a})S(═O)₂R^{10b}, —OR¹⁰, —S(═O)₂R¹⁰, —S(═O)₂N(H)R¹⁰ or —S(═O)₂N(R^{10a})R^{10b},
   said phenyl- or heteroaryl- group being optionally substituted one or more times, identically or differently, with a group selected from: halo-, cyano-, C₁-C₃-alkyl-, fluoro-C₁-C₃-alkyl-, C₁-C₃-alkoxy-;
R⁶ represents a hydrogen atom;
R⁷ represents a group selected from: oxo, C₁-C₄-alkyl-, C₃-C₇-cycloalkyl-, 4- to 7-membered heterocycloalkyl-, fluoro-C₁-C₄-alkyl-, hydroxy-C₁-C₄-alkyl-, cyano-C₁-C₄-alkyl-, C₂-C₄-alkenyl-, C₁-C₄-alkoxy-, fluoro-C₁-C₄-alkoxy-, —OH, —CN, halo-, —C(═O)R⁸, —C(═O)—O—R⁸, —C(═O)N(R^{8a})R^{8b}, —N(R^{10a})R^{10b}, —S(═O)₂R⁸, —S(═O)(═NR¹¹)—R¹⁰, phenyl-, 5- to 6-membered heteroaryl-;
R⁸ represents a hydrogen atom or a C₁-C₆-alkyl-, fluoro-C₁-C₃-alkyl-, cyano-C₁-C₄-alkyl-, C₁-C₃-alkoxy-C₁-C₃-alkyl-, C₃-C₇-cycloalkyl-, phenyl-, 5- to 6-membered heteroaryl- or benzyl- group;
R^{8a}, R^{8b}
   represent, independently from each other, a hydrogen atom, or a C₁-C₆-alkyl-, C₃-C₇-cycloalkyl-, (C₃-C₇-cycloalkyl)-(L³)-, 4- to 10-membered heterocycloalkyl-,
   (4- to 10-membered heterocycloalkyl)-(L³)-, phenyl-, heteroaryl-, phenyl-(L³)-, (phenyl)-O-(L³)-, heteroaryl-(L³)-, or
   (aryl)-(4- to 10-membered heterocycloalkyl)- group;
   said C₁-C₆-alkyl-, C₃-C₇-cycloalkyl-, (C₃-C₇-cycloalkyl)-(L³)-, 4- to 10-membered heterocycloalkyl-, (4- to 10-membered heterocycloalkyl)-(L³)-, phenyl-, heteroaryl-, phenyl-(L³)-, (phenyl)-O-(L³)-, heteroaryl-(L³)-, and (aryl)-(4- to 10-membered heterocycloalkyl)- group being optionally substituted one or more times, identically or differently, with R⁹;
      or
R^{8a} and R^{8b}, together with the nitrogen atom they are attached to,
   represent a 4- to 10-membered heterocycloalkyl-group, said 4- to 10-membered heterocycloalkyl- group being optionally substituted one or more times, identically or differently, with R⁹;
R⁹ represents a halogen atom, or a oxo, C₁-C₃-alkyl-, fluoro-C₁-C₃-alkyl-, hydroxy-C₁-C₃-alkyl-, —CN, —C(═O)R¹⁰, —C(═O)N(H)R¹⁰, —C(═O)N(R^{10a})R^{10b}, —C(═O)O—R¹⁰, —N(R^{10a})R^{10b}, —NO₂, —N(H)C(═O)R¹⁰, —N(R^{10a})C(═O)R^{10b}, —N(H)S(═O)₂R¹⁰, —N(R^{10a})S(═O)₂R^{10b}, —OR¹⁰, —S(═O)₂R¹⁰, —S(═O)₂N(H)R, —S(═O)₂N(R^{10a})R^{1b} or a tetrazolyl- group;
R¹⁰, R^{10a}, R^{10b}, R^{10c}
   represent, independently from each other, a hydrogen atom or a group selected from: C₁-C₃-alkyl-, fluoro-C₁-C₃-alkyl-, hydroxy-C₁-C₃-alkyl-, C₁-C₃-alkoxy-C₁-C₃-alkyl-, C₃-C₇-cycloalkyl-, said C₁-C₃-alkyl- group being optionally substituted once with —N(R¹²)R^{12a};
      or
R^{10a} and R^{10b}, together with the nitrogen atom they are attached to,
   represent a 4- to 7-membered heterocycloalkyl- group, said 4- to 7-membered heterocycloalkyl-group being optionally substituted one or more times, identically or differently, with R¹³;
R¹¹ represents a hydrogen atom or a cyano- or —C(═O)O—R¹⁰ group;
R¹², R^{12a}
   represent, independently from each other, a hydrogen atom or a C₁-C₃-alkyl- group,
   or
R¹², R^{12a},
   together with the nitrogen atom they are attached to, represent a 4- to 7-membered heterocycloalkyl- group;
R¹³ represents a halogen atom or a cyano, hydroxy, oxo, C₁-C₃-alkyl-, trifluoromethyl-, —C(═O)R¹⁰ or —C(═O)O—R¹⁰ group;
L¹ represents a group selected from: —C₁-C₄-alkylene-, —C(phenyl)(H)—, —CH₂—CH₂—O—, —CH₂—C(═O)—N(H)—, —CH₂—C(═O)—N(R^{10a})—;
L² represents a group selected from: —CH₂—, —CH₂—CH₂—, —CH₂—CH₂—CH₂—;
L³ represents a —C₁-C₄-alkylene- group;
p is an integer of 0 or 1;
   with the proviso that at least one of R¹ and R² is different from iso-propyl-,
   or a tautomer, a stereoisomer, an N-oxide, a hydrate, a solvate, or a salt thereof, or a mixture of same.

In a particularly preferred embodiment, the present invention relates to compounds of general formula (I):

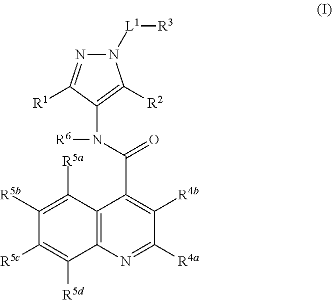

in which:
R¹ represents a C₁-C₃-alkyl-, trifluoromethyl- or cyano- group;
R² represents a methyl-, ethyl- or trifluoromethyl- group;
R³ represents a group selected from: phenyl- or 5- to 6-membered heteroaryl-;
   wherein said phenyl- or 5- to 6-membered heteroaryl- group is optionally substituted, one or more times, identically or differently, with -(L²)ₚ-R⁷,
   or
R³ represents a group

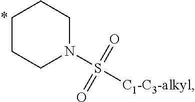

wherein * represents the point of attachment to the rest of the molecule;
R^{4a} represents a group selected from: C₁-C₃-alkyl-, fluoro-C₁-C₃-alkyl-, C₁-C₃-alkoxy-, C₃-C₅-cycloalkyl, —C(═O)N(R^{10a})R^{10b}, —SR¹⁰, —S(═O)—R¹⁰, —S(═NR¹¹)—R¹⁰, —S(═O)₂—R¹⁰, —S(═O)₂—N(R^{10a})R^{10b}, —N(R^{10a})R^{10b};
R^{4b} represents a hydrogen atom;
R^{5a}, R^{5b}, R^{5c}, R^{5d}
   independently from each other represent a hydrogen atom, a halogen atom or a group selected from:
   cyano-, C₁-C₃-alkyl-, C₁-C₃-alkoxy-, —N(R^{10a})R^{10b}, —OR¹⁰;
R⁶ represents a hydrogen atom;
R⁷ represents a group selected from: C₁-C₃-alkyl-, cyclopropyl-, trifluoromethyl-, C₁-C₃-alkoxy-, trifluoromethoxy-, —CN, fluoro-, chloro-, —C(═O)—C₁-C₃-alkyl, —C(═O)N(R^{8a})R^{8b}, —S(═O)₂)—C₁-C₃-alkyl;
R^{8a}, R^{8b}
   represent, independently from each other, a hydrogen atom, or a C₁-C₆-alkyl-, C₃-C₇-cycloalkyl-, 4- to 10-membered heterocycloalkyl-, (4- to 10-membered heterocycloalkyl)-(L³)-, phenyl-, heteroaryl-, phenyl-(L³)- or heteroaryl-(L³)- group;
   said C₁-C₆-alkyl-, C₃-C₇-cycloalkyl-, 4- to 10-membered heterocycloalkyl-, (4- to 10-membered heterocycloalkyl)-(L³)-, phenyl-, heteroaryl-, phenyl-(L³)-, and heteroaryl-(L³)- group being optionally substituted one or more times, identically or differently, with R⁹;
      or
R^{8a} and R^{8b}, together with the nitrogen atom they are attached to, represent a 4- to 7-membered heterocycloalkyl-group;
R⁹ represents a halogen atom, or a C₁-C₃-alkyl-, fluoro-C₁-C₃-alkyl-, hydroxy-C₁-C₃-alkyl-, —CN, —C(═O)R¹⁰, —C(═O)N(H)R¹⁰, —C(═O)N(R^{10a})R^{10b}, —N(R^{10a})R^{10b}, —N(H)C(═O)R¹⁰, —N(R^{10a})C(═O)R^{10b}, —OR¹⁰, group;
R¹⁰, R^{10a}, R^{10b}, R^{10c}
   represent, independently from each other, a hydrogen atom or a group selected from: C₁-C₃-alkyl-, hydroxy-C₁-C₃-alkyl-, C₁-C₃-alkoxy-C₁-C₃-alkyl-;
      or
R^{10a} and R^{10b}, together with the nitrogen atom they are attached to,
   represent a 4- to 7-membered heterocycloalkyl- group;
L¹ represents a group selected from: —C₁-C₄-alkylene-, —CH₂—CH₂—O—;
L² represents a group selected from: —CH₂—, —CH₂—CH₂—;
L³ represents a —C₁-C₄-alkylene- group;
p is an integer of 0 or 1;
   or a tautomer, a stereoisomer, an N-oxide, a hydrate, a solvate, or a salt thereof, or a mixture of same.

In another particularly preferred embodiment, the present invention relates to compounds of general formula (I):

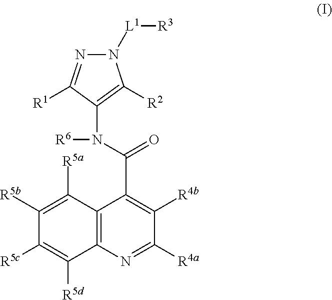

in which:
R¹ represents a C₁-C₃-alkyl-, trifluoromethyl- or cyano- group;
R² represents a methyl-, ethyl- or trifluoromethyl- group;
R³ represents a phenyl- group which is optionally substituted, one or more times, identically or differently, with a group selected from C₁-C₃-alkyl-, trifluoromethyl-, cyanomethyl-, methoxymethyl-, C₁-C₃-alkoxy-, trifluoromethoxy-, —CN, fluoro-, chloro-, —C(═O)—C₁-C₃-alkyl, —C(═O)N(R^{8a})R^{8b}, —S(═O)₂—C₁-C₃-alkyl;
   or
R³ represents a 5- to 6-membered heteroaryl- group which is optionally substituted, one or more times, identically or differently, with a group selected from C₁-C₃-alkyl-, cyclopropyl-, C₁-C₃-alkoxy-, —CN, —C(═O)N(R^{8a})R^{8b};
R^{4a} represents a group selected from: iso-propyl-, trifluoromethyl-, methoxy-, cyclopropyl-, —C(═O)—NH₂;
R^{4b} represents a hydrogen atom;
R^{5a}, R^{5c}, R^{5d}
   independently from each other represent a hydrogen atom, a fluoro atom or a chloro atom;
R^{5b} represents a hydrogen atom, a fluoro atom, a chloro atom, a bromo atom or a group selected from:
   cyano-, methyl-, methoxy-, —N(H)—CH₂—CH₂—OCH₃, and N-piperidinyl-
R⁶ represents a hydrogen atom;
R^{8a}, R^{8b}
   represent, independently from each other, a hydrogen atom, or a C₁-C₄-alkyl-, C₃-C₅-cycloalkyl-, 4- to 7-membered heterocycloalkyl-, (4- to 7-membered heterocycloalkyl)-(L³)-, phenyl- or heteroaryl-(L³)- group;
   said C₁-C₄-alkyl-, C₃-C₅-cycloalkyl-, 4- to 7-membered heterocycloalkyl-, (4- to 7-membered heterocycloalkyl)-(L³)-, phenyl- or heteroaryl-(L³)- group being optionally substituted one or more times, identically or differently, with R⁹;
      or
R^{8a} and R^{8b}, together with the nitrogen atom they are attached to,
   represent a 4- to 7-membered heterocycloalkyl-group;
R⁹ represents a halogen atom, or a C₁-C₃-alkyl-, —CN, —C(═O)NH₂ or —OH group;
L¹ represents a group selected from: —C₁-C₂-alkylene-, —CH₂—CH₂—O—;
L³ represents a —C₁-C₃-alkylene- group;
   or a tautomer, a stereoisomer, an N-oxide, a hydrate, a solvate, or a salt thereof, or a mixture of same.

In another particularly preferred embodiment, the present invention relates to compounds of general formula (I):

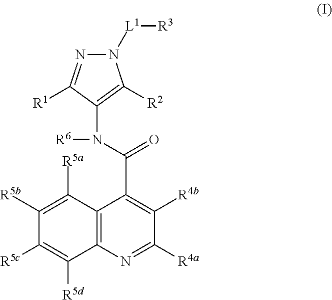

in which:
R¹ represents a methyl- or trifluoromethyl- group;
R² represents a methyl- group;
R³ represents a phenyl- group which is substituted, one or more times, identically or differently, with a group selected from
   methoxy-, —CN, fluoro-,
   or
R³ represents a pyridyl- or pyrimidyl- group which is substituted once with a group selected from
   methoxy-, —CN,
   or
R³ represents a 5-membered heteroaryl- group selected from isoxazolyl-, oxadiazolyl- and thienyl-, which is substituted once with a group selected from C₁-C₃-alkyl-, cyclopropyl-, —CN;
R^{4a} represents a —C(═O)—NH₂ group;
R^{4b} represents a hydrogen atom;
R^{5a}, R^{5b}, R^{5c}, R^{5d}
   independently from each other represent a hydrogen atom, a fluoro atom or a chloro atom;
R⁶ represents a hydrogen atom;
L¹ represents a group selected from: —C₁-C₂-alkylene-, —CH₂—CH₂—O—;
   or a tautomer, a stereoisomer, an N-oxide, a hydrate, a solvate, or a salt thereof, or a mixture of same.

In another particularly preferred embodiment, the present invention relates to compounds of general formula (I):

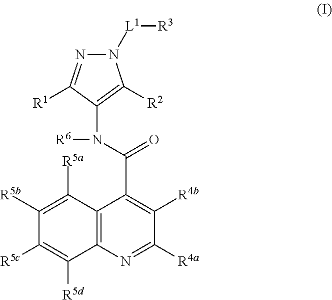

in which:
R¹ represents a methyl- or trifluoromethyl- group;
R² represents a methyl- group;
R³ represents a phenyl- group which is substituted, one or more times, identically or differently, with a group selected from methoxy-, —CN, fluoro-,
   or
R³ represents a pyridyl- or pyrimidyl- group which is substituted once with a group selected from methoxy-, —CN;
R^{4a} represents a —C(═O)—NH₂ group;
R^{4b} represents a hydrogen atom;
R^{5a}, R^{5b}, R^{5c}, R^{5d}
   independently from each other represent a hydrogen atom, a fluoro atom or a chloro atom;
R⁶ represents a hydrogen atom;
L¹ represents a —CH₂— group;
   or a tautomer, a stereoisomer, an N-oxide, a hydrate, a solvate, or a salt thereof, or a mixture of same.

In another particularly preferred embodiment, the present invention relates to compounds of general formula (I):

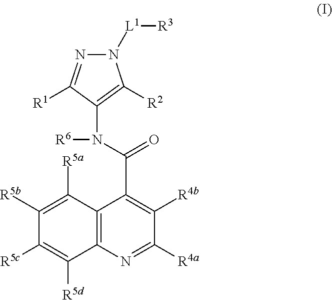

in which:
R¹ represents a methyl- or trifluoromethyl- group;
R² represents a methyl- group;
R³ represents an isoxazolyl-group which is substituted once with a group selected from C₁-C₃-alkyl-, cyclopropyl-;
R^{4a} represents a —C(═O)—NH₂ group;
R^{4b} represents a hydrogen atom;
R^{5a}, R^{5b}, R^{5c}, R^{5d}
   independently from each other represent a hydrogen atom, a fluoro atom or a chloro atom;
R⁶ represents a hydrogen atom;
L¹ represents a —CH₂— group;
   or a tautomer, a stereoisomer, an N-oxide, a hydrate, a solvate, or a salt thereof, or a mixture of same.

In accordance with another aspect, the present invention covers methods of preparing compounds of the present invention, said methods comprising the steps as described in the Experimental Section herein.

In a preferred embodiment, the present invention relates to a method of preparing compounds of general formula (I), supra, in which method an intermediate compound of general formula (II):

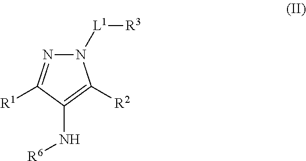

in which R¹, R², R³, R⁶ and L¹ are as defined for the compounds of general formula (I), supra;
is allowed to react with a compound of general formula (III):

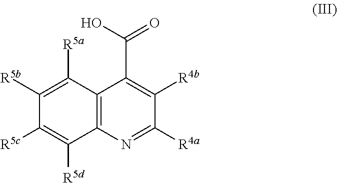

in which R^{4a}, R^{4b}, R^{5a}, R^{5b}, R^{5c}, and R^{5d} are as defined for the compounds of general formula (I), supra;
thus providing a compound of general formula (I):

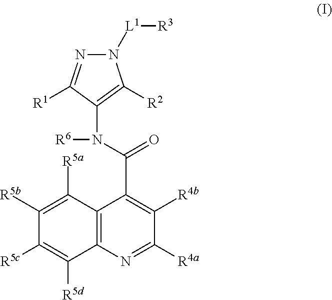

in which R¹, R², R³, R^{4a}, R^{4b}, R^{5a}, R^{5b}, R^{5b}, R^{5d}, R⁶, and L¹ are as defined for the compounds of general formula (I), supra.

In accordance with a further aspect, the present invention covers intermediate compounds which are useful in the preparation of compounds of the present invention of general formula (I), particularly in the method described herein.

In particular, the present invention covers compounds of general formula (II):

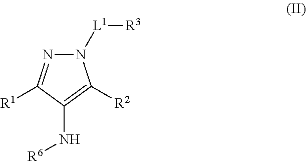

in which R¹, R², R³, R⁶ and L¹ are as defined for the compounds of general formula (I), supra.

In another preferred embodiment, the present invention covers intermediate compounds which are useful in the preparation of compounds of the present invention of general formula (I), particularly in the method described herein.

In particular, the present invention covers compounds of general formula (III):

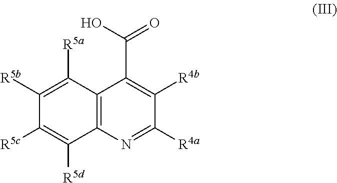

in which R^{4a}, R^{4b}, R^{5a}, R^{5b}, R^{5c}, and R^{5d} are as defined for the compounds of general formula (I), supra.

In accordance with yet another aspect, the present invention covers the use of the intermediate compounds of general formula (II):

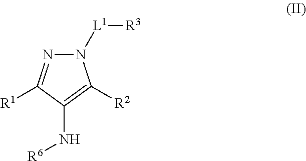

in which R¹, R², R³, R⁶ and L¹ are as defined for the compounds of general formula (I), supra;
for the preparation of a compound of general formula (I) as defined supra.

In another preferred embodiment, the present invention covers the use of the intermediate compounds of general formula (III):

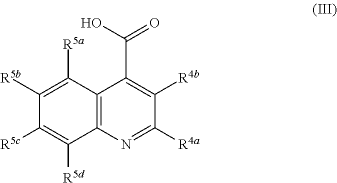

in which R^{4a}, R^{4b}, R^{5a}, R^{5b}, R^{5c}, and R^{5d} are as defined for the compounds of general formula (I), supra;
for the preparation of a compound of general formula (I) as defined supra.

As one of ordinary skill in the art is aware of, the methods described above may comprise further steps like e.g. the introduction of a protective group and the cleavage of the protective group.

This invention also relates to pharmaceutical compositions containing one or more compounds of the present invention. These compositions can be utilised to achieve the desired pharmacological effect by administration to a patient in need thereof. A patient, for the purpose of this invention, is a mammal, including a human, in need of treatment for the particular condition or disease. Therefore, the present invention includes pharmaceutical compositions that are comprised of a pharmaceutically acceptable carrier and a pharmaceutically effective amount of a compound, or salt thereof, of the present invention. A pharmaceutically acceptable carrier is preferably a carrier that is relatively non-toxic and innocuous to a patient at concentrations consistent with effective activity of the active ingredient so that any side effects ascribable to the carrier do not vitiate the beneficial effects of the active ingredient. A pharmaceutically effective amount of compound is preferably that amount which produces a result or exerts an influence on the particular condition being treated. The compounds of the present invention can be administered with pharmaceutically-acceptable carriers well known in the art using any effective conventional dosage unit forms, including immediate, slow and timed release preparations, orally, parenterally, topically, nasally, ophthalmically, optically, sublingually, rectally, vaginally, and the like.

The compounds of this invention can be administered as the sole pharmaceutical agent or in combination with one or more other pharmaceutical agents where the combination causes no unacceptable adverse effects. The present invention relates also to such combinations. For example, the compounds of this invention can be combined with known anti-hyper-proliferative or other indication agents, and the like, as well as with admixtures and combinations thereof. Other indication agents include, but are not limited to, anti-angiogenic agents, mitotic inhibitors, alkylating agents, anti-metabolites, DNA-intercalating antibiotics, growth factor inhibitors, cell cycle inhibitors, enzyme inhibitors, toposisomerase inhibitors, biological response modifiers, or anti-hormones.

Preferred additional pharmaceutical agents are: 131I-chTNT, abarelix, abiraterone, aclarubicin, aldesleukin, alemtuzumab, alitretinoin, altretamine, aminoglutethimide, amrubicin, amsacrine, anastrozole, arglabin, arsenic trioxide, asparaginase, azacitidine, basiliximab, BAY 80-6946, BAY 1000394, BAY 86-9766 (RDEA 119), belotecan, bendamustine, bevacizumab, bexarotene, bicalutamide, bisantrene, bleomycin, bortezomib, buserelin, busulfan, cabazitaxel, calcium folinate, calcium levofolinate, capecitabine, carboplatin, carmofur, carmustine, catumaxomab, celecoxib, celmoleukin, cetuximab, chlorambucit, chlormadinone, chlormethine, cisplatin, cladribine, clodronic acid, clofarabine, crisantaspase, cyclophosphamide, cyproterone, cytarabine, dacarbazine, dactinomycin, darbepoetin alfa, dasatinib, daunorubicin, decitabine, degarelix, denileukin diftitox, denosumab, destorelin, dibrospidium chloride, docetaxel, doxifluridine, doxorubicin, doxorubicin+estrone, ecutizumab, edrecolomab, elliptinium acetate, eltrombopag, endostatin, enocitabine, epirubicin, epitiostanol, epoetin atfa, epoetin beta, eptaplatin, eributin, erlotinib, estradiol, estramustine, etoposide, everolimus, exemestane, fadrozole, filgrastim, fludarabine, fluorouracil, flutamide, formestane, fotemustine, fulvestrant, gallium nitrate, ganirelix, gefitinib, gemcitabine, gemtuzumab, glutoxim, goseretin, histamine dihydrochioride, histrelin, hydroxycarbamide, I-125 seeds, ibandronic acid, ibritumomab tiuxetan, idarubicin, ifosfamide, imatinib, imiquimod, improsutfan, interferon alfa, interferon beta, interferon gamma, ipilimumab, irinotecan, ixabepilone, lanreotide, apatinib, lenalidomide, lenograstim, lentinan, letrozole, leuprorelin, evamisole, isuride, lobaplatin, tomustine, lonidamine, masoprocol, medroxyprogesterone, megestrol, melphatan, mepitiostane, mercaptopurine, methotrexate, methoxsalen, Methyl aminolevutinate, methyltestosterone, mifamurtide, miltefosine, miriplatin, mitobronitol, mitoguazone, mitolactot, mitomycin, mitotane, mitoxantrone, nedaplatin, neLarabine, nilotinib, nilutamide, nimotuzumab, nimustine, nitracrine, ofatumumab, omeprazote, oprelvekin, oxaliplatin, p53 gene therapy, pactitaxel, palifermin, palladium-103 seed, pamidronic acid, panitumumab, pazopanib, pegaspargase, PEG-epoetin beta (methoxy PEG-epoetin beta), pegfilgrastim, peginterferon alfa-2b, pemetrexed, pentazocine, pentostatin, peplomycin, perfosfamide, picibanit, pirarubicin, plerixafor, plicamycin, poliglusam, potyestradiot phosphate, polysaccharide-K, porfimer sodium, pralatrexate, prednimustine, procarbazine, quinagotide, raloxifene, raltitrexed, ranimustine, razoxane, regorafenib, risedronic acid, rituximab, romidepsin, romiplostim, sargramostim, sipuleucel-T, sizofiran, sobuzoxane, sodium glycididazote, sorafenib, streptozocin, sunitinib, talaporfin, tamibarotene, tamoxifen, tasonermin, teceleukin, tegafur, tegafur+gimeracil+oteracil, temoporfin, temozolomide, temsirolimus, teniposide, testosterone, tetrofosmin, thalidomide, thiotepa, thymalfasin, tioguanine, tociLizumab, topotecan, toremifene, tositumomab, trabectedin, trastuzumab, treosulfan, tretinoin, trilostane, triptoretin, trofosfamide, tryptophan, ubenimex, valrubicin, vandetanib, vapreotide, vemurafenib, vinblastine, vincristine, vindesine, vinflunine, vinorelbine, vorinostat, vorozote, yttrium-90 glass microspheres, zinostatin, zinostatin stimalamer, zoledronic acid, zorubicin.

Optional anti-hyper-proliferative agents which can be added to the composition include but are not limited to compounds listed on the cancer chemotherapy drug regimens in the 11^{th} Edition of the Merck Index, (1996), which is hereby incorporated by reference, such as asparaginase, bleomycin, carboplatin, carmustine, chlorambucil, cisplatin, colaspase, cyclophosphamide, cytarabine, dacarbazine, dactinomycin, daunorubicin, doxorubicin (adriamycine), epirubicin, etoposide, 5-fluorouracil, hexamethylmelamine, hydroxyurea, ifosfamide, irinotecan, leucovorin, Lomustine, mechiorethamine, 6-mercaptopurine, mesna, methotrexate, mitomycin C, mitoxantrone, prednisotone, prednisone, procarbazine, raloxifen, streptozocin, tamoxifen, thioguanine, topotecan, vinblastine, vincristine, and vindesine.

Other anti-hyper-proliferative agents suitable for use with the composition of the invention include but are not limited to those compounds acknowledged to be used in the treatment of neoplastic diseases in Goodman and Gilman's The Pharmacological Basis of Therapeutics (Ninth Edition), editor Molinoff et al., publ. by McGraw-Hill, pages 1225-1287, (1996), which is hereby incorporated by reference, such as aminoglutethimide, L-asparaginase, azathioprine, 5-azacytidine cladribine, busulfan, diethylstilbestrol, 2′,2′-difluorodeoxycytidine, docetaxel, erythrohydroxynonyl adenine, ethinyl estradiol, 5-fluorodeoxyuridine, 5-fluorodeoxyuridine monophosphate, fludarabine phosphate, fluoxymesterone, flutamide, hydroxyprogesterone caproate, idarubicin, interferon, medroxyprogesterone acetate, megestrol acetate, melphalan, mitotane, paclitaxel, pentostatin, N-phosphonoacetyl-L-aspartate (PALA), plicamycin, semustine, teniposide, testosterone propionate, thiotepa, trimethylmelamine, uridine, and vinorelbine.

Other anti-hyper-proliferative agents suitable for use with the composition of the invention include but are not limited to other anti-cancer agents such as epothilone and its derivatives, irinotecan, raloxifen and topotecan.

The compounds of the invention may also be administered in combination with protein therapeutics. Such protein therapeutics suitable for the treatment of cancer or other angiogenic disorders and for use with the compositions of the invention include, but are not limited to, an interferon (e.g., interferon .alpha., .beta., or .gamma.) supraagonistic monoclonal antibodies, Tuebingen, TRP-1 protein vaccine, Colostrinin, anti-FAP antibody, YH-16, gemtuzumab, infliximab, cetuximab, trastuzumab, denileukin diftitox, rituximab, thymosin alpha 1, bevacizumab, mecasermin, mecasermin rinfabate, oprelvekin, natalizumab, rhMBL, MFE-CP1+ZD-2767-P, ABT-828, ErbB2-specific immunotoxin, SGN-35, MT-103, rinfabate, AS-1402, B43-genistein, L-19 based radioimmunotherapeutics, AC-9301, NY-ESO-1 vaccine, IMC-1C11, CT-322, rhCC₁₀, r(m)CRP, MORAb-009, aviscumine, MDX-1307, Her-2 vaccine, APC-8024, NGR-hTNF, rhH1.3, IGN-311, Endostatin, volociximab, PRO-1762, lexatumumab, SGN-40, pertuzumab, EMD-273063, L19-IL-2 fusion protein, PRX-321, CNTO-328, MDX-214, tigapotide, CAT-3888, labetuzumab, alpha-particle-emitting radioisotope-linked lintuzumab, EM-1421, HyperAcute vaccine, tucotuzumab celmoleukin, galiximab, HPV-16-E7, Javelin—prostate cancer, Javelin—melanoma, NY-ESO-1 vaccine, EGF vaccine, CYT-004-MelQbG10, WT1 peptide, oregovomab, ofatumumab, zalutumumab, cintredekin besudotox, WX-G250, ALbuferon, aflibercept, denosumab, vaccine, CTP-37, efungumab, or 131I-chTNT-1/B. Monoclonal antibodies useful as the protein therapeutic include, but are not limited to, muromonab-CD3, abciximab, edrecolomab, daclizumab, gentuzumab, alemtuzumab, ibritumomab, cetuximab, bevicizumab, efalizumab, adalimumab, omalizumab, muromomab-CD3, rituximab, daclizumab, trastuzumab, palivizumab, basiliximab, and infliximab.

Generally, the use of cytotoxic and/or cytostatic agents in combination with a compound or composition of the present invention will serve to:
(1) yield better efficacy in reducing the growth of a tumor or even eliminate the tumor as compared to administration of either agent alone,
(2) provide for the administration of lesser amounts of the administered chemotherapeutic agents,
(3) provide for a chemotherapeutic treatment that is well tolerated in the patient with fewer deleterious pharmacological complications than observed with single agent chemotherapies and certain other combined therapies,
(4) provide for treating a broader spectrum of different cancer types in mammals, especially humans,
(5) provide for a higher response rate among treated patients,
(6) provide for a longer survival time among treated patients compared to standard chemotherapy treatments,
(7) provide a longer time for tumor progression, and/or
(8) yield efficacy and tolerability results at least as good as those of the agents used alone, compared to known instances where other cancer agent combinations produce antagonistic effects.

The compounds of formula (I), supra, as described and defined herein have surprisingly been found to effectively and selectively inhibit GLUT1 and may therefore be used for the treatment and/or prophylaxis of diseases of uncontrolled cell growth, proliferation and/or survival, inappropriate cellular immune responses, or inappropriate cellular inflammatory responses, or diseases which are accompanied with uncontrolled cell growth, proliferation and/or survival, inappropriate cellular immune responses, or inappropriate cellular inflammatory responses, such as, for example, haematological tumours, solid tumours, and/or metastases thereof, e.g. Leukaemias and myelodysplastic syndrome, malignant lymphomas, head and neck tumours including brain tumours and brain metastases, tumours of the thorax including non-small cell and small cell lung tumours, gastrointestinal tumours, endocrine tumours, mammary and other gynaecological tumours, urological tumours including renal, bladder and prostate tumours, skin tumours, and sarcomas, and/or metastases thereof.

In accordance with another aspect therefore, the present invention covers a compound of general formula (I), or a stereoisomer, a tautomer, an N-oxide, a hydrate, a solvate, or a salt thereof, particularly a pharmaceutically acceptable salt thereof, or a mixture of same, as described and defined herein, for use in the treatment or prophylaxis of a disease, as mentioned supra.

Another particular aspect of the present invention is the use of a compound of general formula (I), described supra, or a stereoisomer, a tautomer, an N-oxide, a hydrate, a solvate, or a salt thereof, particularly a pharmaceutically acceptable salt thereof, or a mixture of same, for the prophylaxis or treatment of a disease.

Another particular aspect of the present invention is the use of a compound of general formula (I) described supra for manufacturing a pharmaceutical composition for the treatment or prophylaxis of a disease.

The compounds of the present invention can be used in particular in therapy and prevention, i.e. prophylaxis, of tumour growth and metastases, especially in solid tumours of all indications and stages with or without pre-treatment of the tumour growth.

Methods of testing for a particular pharmacological or pharmaceutical property are well known to persons skilled in the art.

The present invention relates to a method for using the compounds of the present invention and compositions thereof, to treat mammalian hyper-proliferative disorders. Compounds can be utilized to inhibit, block, reduce, decrease, etc., cell proliferation and/or cell division, and/or produce apoptosis. This method comprises administering to a mammal in need thereof, including a human, an amount of a compound of this invention, or a pharmaceutically acceptable salt, isomer, polymorph, metabolite, hydrate, solvate or ester thereof; etc. which is effective to treat the disorder. Hyper-proliferative disorders include but are not limited, e.g., psoriasis, keloids, and other hyperplasias affecting the skin, benign prostate hyperplasia (BPH), solid tumors, such as cancers of the breast, respiratory tract, brain, reproductive organs, digestive tract, urinary tract, eye, liver, skin, head and neck, thyroid, parathyroid and their distant metastases. Those disorders also include Lymphomas, sarcomas, and Leukemias.

Examples of breast cancer include, but are not limited to invasive ductal carcinoma, invasive Lobular carcinoma, ductal carcinoma in situ, and lobular carcinoma in situ.

Examples of cancers of the respiratory tract include, but are not limited to small-cell and non-small-cell ung carcinoma, as wellt as bronchial adenoma and pleuropulmonary blastoma.

Examples of brain cancers include, but are not limited to brain stem and hypophtalmic glioma, cerebellar and cerebral astrocytoma, medulloblastoma, ependymoma, as well as neuroectodermal and pineal tumor.

Tumors of the male reproductive organs include, but are not limited to prostate and testicular cancer. Tumors of the female reproductive organs include, but are not limited to endometrial, cervical, ovarian, vaginal, and vulvar cancer, as well as sarcoma of the uterus.

Tumors of the digestive tract include, but are not limited to anal, colon, colorectal, esophageal, gallbladder, gastric, pancreatic, rectal, small-intestine, and salivary gland cancers.

Tumors of the urinary tract include, but are not limited to bladder, penile, kidney, renal pelvis, ureter, urethral and human papillary renal cancers.

Eye cancers include, but are not limited to intraocular melanoma and retinoblastoma.

Examples of liver cancers include, but are not limited to hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to squamous cell carcinoma, Kaposi's sarcoma, malignant melanoma, Merkel cell skin cancer, and non-melanoma skin cancer.

Head-and-neck cancers include, but are not limited to laryngeal, hypopharyngeal, nasopharyngeal, oropharyngeal cancer, lip and oral cavity cancer and squamous cell. Lymphomas include, but are not limited to AIDS-related lymphoma, non-Hodgkin's lymphoma, cutaneous T-cell lymphoma, Burkitt Lymphoma, Hodgkin's disease, and Lymphoma of the central nervous system.

Sarcomas include, but are not limited to sarcoma of the soft tissue, osteosarcoma, malignant fibrous histiocytoma, lymphosarcoma, and rhabdomyosarcoma.

Leukemias include, but are not limited to acute myeloid leukemia, acute Lymphoblastic leukemia, chronic lymphocytic leukemia, chronic myelogenous leukemia, and hairy cell leukemia.

These disorders have been well characterized in humans, but also exist with a similar etiology in other mammals, and can be treated by administering pharmaceutical compositions of the present invention.

The term “treating” or “treatment” as stated throughout this document is used conventionally, e.g., the management or care of a subject for the purpose of combating, alleviating, reducing, relieving, improving the condition of, etc., of a disease or disorder, such as a carcinoma.

Based upon standard laboratory techniques known to evaluate compounds useful for the treatment of hyper-proliferative disorders and angiogenic disorders, by standard toxicity tests and by standard pharmacological assays for the determination of treatment of the conditions identified above in mammals, and by comparison of these results with the results of known medicaments that are used to treat these conditions, the effective dosage of the compounds of this invention can readily be determined for treatment of each desired indication. The amount of the active ingredient to be administered in the treatment of one of these conditions can vary widely according to such considerations as the particular compound and dosage unit employed, the mode of administration, the period of treatment, the age and sex of the patient treated, and the nature and extent of the condition treated.

The total amount of the active ingredient to be administered will generally range from about 0.001 mg/kg to about 200 mg/kg body weight per day, and preferably from about 0.01 mg/kg to about 20 mg/kg body weight per day. Clinically useful dosing schedules will range from one to three times a day dosing to once every four weeks dosing. In addition, “drug holidays” in which a patient is not dosed with a drug for a certain period of time, may be beneficial to the overall balance between pharmacological effect and tolerability. A unit dosage may contain from about 0.5 mg to about 1500 mg of active ingredient, and can be administered one or more times per day or less than once a day. The average daily dosage for administration by injection, including intravenous, intramuscular, subcutaneous and parenteral injections, and use of infusion techniques will preferably be from 0.01 to 200 mg/kg of total body weight. The average daily rectal dosage regimen will preferably be from 0.01 to 200 mg/kg of total body weight. The average daily vaginal dosage regimen will preferably be from 0.01 to 200 mg/kg of total body weight. The average daily topical dosage regimen will preferably be from 0.1 to 200 mg administered between one to four times daily. The transdermal concentration will preferably be that required to maintain a daily dose of from 0.01 to 200 mg/kg. The average daily inhalation dosage regimen will preferably be from 0.01 to 100 mg/kg of total body weight.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

### General Synthesis of Compounds of General Formula (I) of the Present Invention

The following paragraphs outline a variety of synthetic approaches suitable to prepare compounds of the general formula (I), and intermediates useful for their synthesis.

In addition to the routes described below, also other routes may be used to synthesise the target compounds, in accordance with common general knowledge of a person skilled in the art of organic synthesis. The order of transformations exemplified in the following schemes is therefore not intended to be limiting, and suitable synthesis steps from various schemes can be combined to form additional synthesis sequences. In addition, interconversion of any of the substituents, in particular R¹, R², R^{4a}, R^{4b}, R^{5a}, R^{5b}, R^{5c}, R^{5d} or R⁶, as well as of the R⁷ group attached to R³ via -(L²)ₚ-, can be achieved before and/or after the exemplified transformations. These modifications can be such as the introduction of protective groups, cleavage of protective groups, reduction or oxidation of functional groups, halogenation, metallation, metal catalysed coupling reactions, exemplified by but not limited to Suzuki, Sonogashira and Ullmann coupling, ester saponifications, amide coupling reactions, and/or substitution or other reactions known to a person skilled in the art. These transformations include those which introduce a functionality allowing for further interconversion of substituents. Appropriate protective groups and their introduction and cleavage are well-known to a person skilled in the art (see for example T. W. Greene and P. G. M. Wuts in Protective Groups in Organic Synthesis, 3^{rd} edition, Wiley 1999).

Specific examples of said interconversions are described in the subsequent paragraphs. Exemplary reference is being made to several specific protocols in the experimental section, infra, e.g. for conversions of R^{5b}=bromo into a secondary amine (Intermediate 55A), of R^{5d} and R^{5b}=bromo into cyano-(examples 102, 103), and of R^{5b}=bromo into phenyl- (example 166), or —C(═O)—OCH₃ (example 149) by means of palladium catalysed coupling reactions, the conversion of R⁷=—C(═O)—OCH₃ into the corresponding carboxylic acid and multiple carboxamide derivatives by ester hydrolysis, followed by carboxamide coupling e.g. as described in examples 131 to 143, the conversion of R^{4a}=bromo into R^{4a}=ethyl by reaction with a Grignard reagent (example 69), and to the conversion of R⁷=cyano- into a tetrazolyl- group (example 400). Further, it is possible that two or more successive steps may be performed without work-up being performed between said steps, e.g. a “one-pot” reaction, as it is well-known to a person skilled in the art.

Compounds of general formula (I) can be assembled from 4-aminopyrazole derivatives of formula (II), in which R¹, R², R³, R⁶ and L¹ are as defined for the compounds of general formula (I), and quinoline-4-carboxylic acid derivatives of formula (III), in which R^{4a}, R^{4b}, R^{5a}, R^{5b}, R^{5c} and R^{5d} are as defined for the compounds of general formula (I), by means of carboxamide (or peptide) coupling reaction well known to the person skilled in the art, according to Scheme 1. Said coupling reaction can be performed by reaction of compounds of the formulae (II) and (III) in the presence of a suitable coupling reagent, such as HATU (O-(7-azabenzotriazol-1-yl)-N,N,N′,N′-tetramethyluronium hexafluorophosphate), TBTU (O-(benzotriazol-1-yl)-N,N,N′,N′-tetramethyluronium tetrafluoroborate), PyBOP (benzotriazol-1-yl-oxytripyrrolidinophosphonium hexafluorophosphate), or EDC (1-(3-dimethylaminopropyl)-3-ethylcarbodiimide hydrochloride) in combination with HOBt (1-hydroxy-1H-benzotriazole hydrate), in the presence of a base such as an aliphatic or aromatic tertiary amine, preferably a tertiary aliphatic amine of the formula N(C₁-C₄-alkyl)₃, in an appropriate solvent.

Preferred herein is the performance of said carboxamide coupling reaction using O-(benzotriazol-1-yl)-N,N,N′,N′-tetramethyluronium tetrafluoroborate (TBTU) as a coupling agent, in the presence of N,N-diisopropylethylamine as a base, and in tetrahydrofuran as a solvent, within a temperature range from 0° C. to 50° C.

Also preferred herein is the performance of said carboxamide coupling reaction using O-(7-azabenzotriazol-1-yl)-N,N,N′,N′-tetramethyluronium hexafluorophosphate (HATU) as a coupling agent, in the presence of N,N-diisopropylethylamine as a base, and in dimethylsulfoxide as a solvent, within a temperature range from 0° C. to 50° C.

Also preferred herein is the performance of said carboxamide coupling reaction using benzotriazol-1-yl-oxytripyrrolidinophosphonium hexafluorophosphate (PyBOP) as a coupling agent, in the presence of N,N-diisopropylethylamine as a base, and in tetrahydrofuran as a solvent, within a temperature range from 0° C. to 50° C.

The preparation of amides from 4-aminopyrazole derivatives of formula (II), in which R¹, R², R³, R⁶ and L¹ are as defined for the compounds of general formula (I), and quinoline-4-carboxylic acid derivatives of formula (III), in which R^{4a}, R^{4b}, R^{5a}, R^{5b}, R^{5c} and R^{5d} are as defined for the compounds of general formula (I), can furthermore be accomplished, as well known to the person skilled in the art, by converting carboxylic acids of the formula (III) into the corresponding acyl halides, e.g. by reacting with a halogenating agent such as thionyl chloride, oxalyl chloride, or phosphoroxy chloride, and subsequent aminotysis using said 4-aminopyrazole derivatives of formula (II).

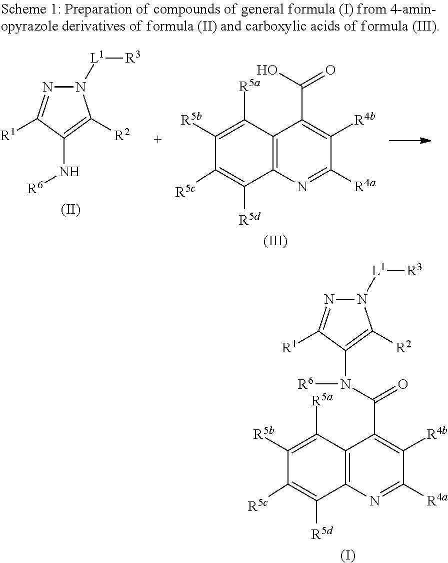

4-Aminopyrazole intermediates and quinazoline-4-carboxylic acid derivatives of formulae (II) and (III) can be prepared using synthetic methods described in more detail as according to Schemes 3, 4 and 5 shown below. Certain quinazoline-4-carboxylic acids are also commercially available in some structural variety.

If aminopyrazole derivatives of formula (II), in which R⁶ represents a hydrogen atom, have been employed in the carboxamide coupling reaction described supra, R⁶ groups different from hydrogen can also be introduced subsequently to said carboxamide coupling reaction by means of deprotonating the resulting compounds of formula (Ia), in which R¹, R², R³, R^{4a}, R^{4b}, R^{5a}, R^{5b}, R^{5c}, R^{5d} and L¹ are as defined for the compounds of general formula (I), with a base such as an alkali metal hydride, preferably sodium hydride, followed by reaction with a compound of the formula (IV), in which LG represents a leaving group, preferably chloro, bromo, or iodo, and in which R⁶ is as defined for the compounds of general formula (I) but different from hydrogen, to give compounds of formula (Ib), as outlined in Scheme 2.

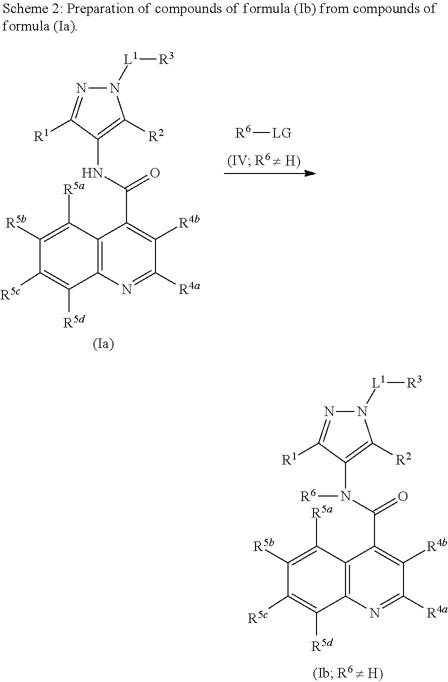

Compounds of formula (IV) are well known to the person skilled in the art and are readily commercially available.

Intermediate 4-aminopyrazole derivatives of formula (II) are available e.g. by reaction of 4-nitropyrazole derivatives of the formula (V), in which R¹ and R² are as defined for the compounds of general formula (I), with compounds of the formula (VI), in which R³ and L¹ are as defined for the compounds of general formula (I), and in which LG represents a leaving group, preferably chloro, bromo, or iodo, in the presence of a suitable base such as an alkali carbonate, preferably cesium carbonate, to give N-1-substituted nitropyrazole intermediates of formula (VII). As another suitable base, 1,8-diazabicyclo(5.4.0)undec-7-ene can be used to perform said alkylation reaction. Alternatively, the nitro group can be introduced after substitution of pyrazole N-1 with -L¹-R³ described above; for said inverse synthetic route see e.g. the protocols describing the preparation of Intermediate 30B in the experimental section, infra.

In cases where R¹ and R² are different from each other, said nitropyrazole intermediates of formula (VII) are formed as mixtures of regioisomers, as a result of the tautomery featured by the pyrazole core. Said mixtures can be separated into pure regioisomers by methods known to the person skilled in the art, such as column chromatography on silica gel, or by preparative HPLC, either directly following the reaction, or on a later or final stage.

Said compounds of formula (VII) can subsequently be reduced, using reduction methods well known to the person skilled in the art, to give primary amines of formula (IIa). Said reduction methods encompass the use of palladium catalysed hydrogenation, using elemental hydrogen or alternative hydrogen sources such as ammonium formate, and the use of zinc dust or powdered iron in the presence of acetic acid, or the use of tin (II) chloride e.g. in ethanol as a solvent. The latter reagents are preferably used if the substrate contains functional groups vulnerable to catalytic hydrogenation, such as cyano-, bromo or chloro, in particular if attached to an aromatic ring.

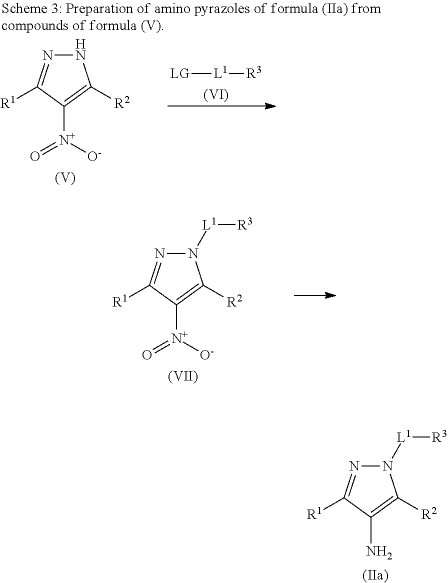

4-Nitropyrazoles of the formula (V) are well known to the person skilled in the art (see e.g. ethyl 3-methyl-4-nitro-1H-pyrazole-5-carboxylate see Journal of Organic Chemistry 1956, p. 833; 3-methyl-4-nitro-1H-pyrazole-5-carbonitrile see Journal of Heterocyclic Chemistry 1970, p. 863; 3-methyl-4-nitro-1H-pyrazole-5-carboxamide see Journal of Organic Chemistry 1956, p. 833 or U.S. Pat. No. 4,282,361 (1981); N,3-dimethyl-4-nitro-1H-pyrazole-5-carboxamide see Chinese Chemical Letters 2012, p. 669; N,N,3-trimethyl-4-nitro-1H-pyrazole-5-carboxamide see DE1945430 (1968)) and are also commercially available in certain cases (e.g. ethyl 3-methyl-4-nitro-1H-pyrazole-5-carboxylate at Fluorochem, Matrix, Oakwood; 3,5-dimethyl-4-nitro-1H-pyrazole at ABCR; 5-methyl-4-nitro-3-(trifluoromethyl)-1H-pyrazole at ABCR, Fluorochem, Matrix).

In general, the nitro group can be introduced into pyrazole derivatives lacking substitution at C-4, by treating 3,4-disubstituted pyrazoles with sulfuric and nitric acid (see e.g. Intermediates 1D-5D), to give 4-nitropyrazoles of formula (V).

R⁶ groups different from hydrogen can be either be introduced at later stage, as outlined in Scheme 2, or they may be introduced into primary amines by means of reductive amination reactions well known to the person skilled in the art, e.g. by reaction of said primary amines of formula (IIa) with suitable aldehydes or ketones, followed by reduction e.g. with sodium cyanoborohydride.

Quinoline-4-carboxylic acid derivatives of formula (III), if not commercially available, can be prepared readily from indole-2,3-dione precursors (see e.g. Monatshefte für Chemie 2013, p. 391; Chinese Chemical Letters 2010, p. 35; The Pfitzinger Reaction. (Review) in Chemistry of Heterocyclic Compounds, Vol 40 (2004), Issue 3, pp 257) of formula (VIII), in which R^{5a}, R^{5b}, R^{5c} and R^{5d} are as defined for the compounds of general formula (I), by reaction with carbonyl compounds of formula (IX), in which R^{4a} and R^{4b} are as defined for the compounds of general formula (I), in an aqueous buffered solvent e.g. comprising sodium hydroxide, sodium acetate, acetic acid and water, at an elevated temperature, to directly give compounds of formula (III), as outlined in Scheme 4.

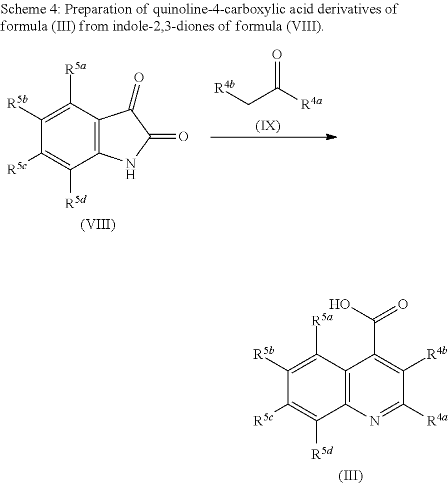

Indole-2,3-diones of formula (VIII) are well known to the person skilled in the art and are either commercially available or can be prepared by methods described e.g. in Chinese Chemical Letters, 2013, p. 929; J. Med. Chem. 2006, p. 4638. Carbonyl compounds of formula (IX) can be purchased commercially in wide structural variety.

The chemical reactivity of groups R^{4a} present in compounds of formula (III) can be modulated as a result of the neighbouring ring nitrogen atom, thus allowing for chemoselective manipulation of R^{4a}. This may be exemplified by (but is not limited to) the synthesis of a subset of said quinoline-4-carboxylic acid derivatives described by formula (IIId), in which R^{4a} is represented by a group —C(═O)N(R^{10a})R^{10b}, as outlined in Scheme 5. Diacids of the formula (IIIa), which are available e.g. by reacting pyruvic acid with an indole-2,3-dione of formula (V) according to Scheme 4, can be converted readily into the respective diesters of formula (IIIb), in which R^{4b}, R^{5a}, R^{5b}, R^{5c} and R^{5d} are as defined for the compounds of general formula (I), and in which R^{E} represents C₁-C₃-alkyl-, by conversion of the carboxy groups into acyl halides using methods well known to the person skilled in the art, e.g. by reaction with thionyl chloride, followed by solvolysis in an aliphatic alcohol of the formula C₁-C₃-alkyl-OH, preferably methanol. The resulting diesters of formula (IIIb) are then reacted with an amine of formula (X), in which R^{10a} and R^{10b} are as defined for the compounds of general formula (I), to give monoamides of formula (IIIc), which are subsequently subjected to ester hydrolysis by methods known to the person skilled in the art, preferably by an alkali hydroxide in an aqueous aliphatic alcohol of the formula C₁-C₃-alkyl-OH, to give the quinoline-4-carboxylic acid derivatives of formula (IIId). The sequence of protocols describing the preparation of Intermediate 2A in the experimental part below constitute an instructive example for this reaction sequence.

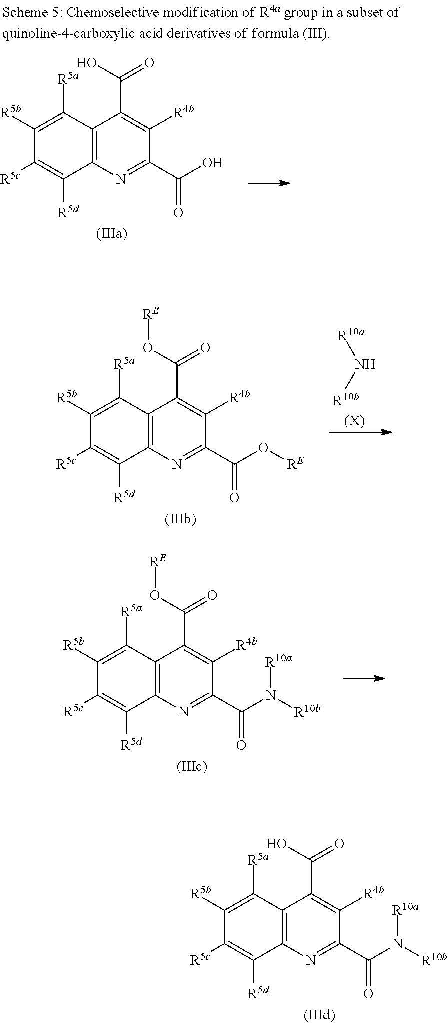

An alternative synthetic approach to the compounds of the general formula (I), which is particularly suitable for the preparation or multiple derivatives featuring different -L¹-R³ moieties by introducing said -L¹-R³ moieties on late stage, is outlined in Scheme 6. 4-Aminopyrazoles of formula (X), in which R¹, R² and R⁶ are as defined for the compounds of general formula (I), and quinoline-4-carboxylic acid derivatives of formula (III), in which R^{4a}, R^{4b}, R^{5a}, R^{5b}, R^{5c} and R^{5d} are as defined for the compounds of general formula (I), are subjected to a carboxamide (or peptide) coupling reaction well known to the person skilled in the art, as discussed supra with regard to Scheme 1, to give intermediate compounds of formula (XI). Said coupling reaction can be performed by reaction of compounds of the formulae (X) and (III) in the presence of a suitable coupling reagent, such as HATU (O-(7-azabenzotriazol-1-yl)-N,N,N′,N′-tetramethyluronium hexafluorophosphate), TBTU (O-(benzotriazol-1-yl)-N,N,N′,N′-tetramethyluronium tetrafluoroborate), PyBOP (benzotriazol-1-yl-oxytripyrrolidinophosphonium hexafluorophosphate), or EDC (1-(3-dimethylaminopropyl)-3-ethylcarbodiimide hydrochloride) in combination with HOBt (1-hydroxy-1H-benzotriazole hydrate), in the presence of a base such as an aliphatic or aromatic tertiary amine, preferably a tertiary aliphatic amine of the formula N(C₁-C₄-alkyl)₃, in an appropriate solvent.

Participation of the pyrazole ring NH in said carboxamide coupling reaction may give rise to the formation of intermediate compounds of formula (XI) as regioisomeric mixtures with the corresponding N1 amides. These can be removed by separation techniques well known to the person skilled in the art, e.g. preparative HPLC either immediately after the coupling, or, preferably, after conversion into the compounds of general formula (I).

Said intermediate compounds of formula (XI) can be converted into the compounds of general formula (I) by reaction with compounds of the formula (VI), in which R³ and L¹ are as defined for the compounds of general formula (I), and in which LG represents a Leaving group, preferably chloro, bromo, or iodo, in the presence of a suitable inorganic base, such as an alkali carbonate, preferably cesium carbonate or an alkali hydride, such as sodium hydride, or an organic base, such as potassium tert.-butoxide or 1,8-diazabicyclo[5.4.0]undec-7-ene.

4-Aminopyrazoles of formula (X) are well known to the person skilled in the art and can be purchased commercially in many cases.

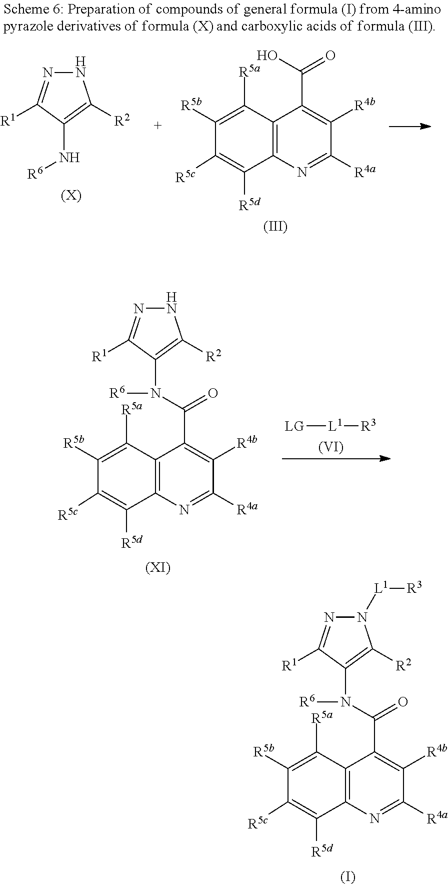

### ABBREVIATIONS

| | |
|---|---|
| DMF | N,N-dimethylformamide |
| HPLC | high performance liquid chromatography |
| HOBt | 1-hydroxy-1H-benzotriazole hydrate |
| UPLC | ultra performance liquid chromatography |
| DAD | diode array detector |
| EDC | 1-(3-dimethylaminopropyl)-3-ethylcarbodiimide hydrochloride |
| ELSD | evaporative light scattering detector |
| ESI | electrospray ionization |
| DLD1 | colorectal adenocarcinoma cells isolated by D.L. Dexter |
| CHO-K1 | chinese hamster ovary K1 cells |
| H460 | lung carcinoma cells |
| RCC | renal cell carcinoma cells |
| VHL | von Hippel-Lindau |
| DMEM | Dulbecco′s modified eagle medium |
| FCS | fetal calf serum |
| HEPES | 4-(2-hydroxyethyl)-1-piperazineethanesulfonic acid |
| HMPA | Hexamethylphosphoramide |
| KRP | Krbes-Ringer phosphate |
| HATU | O-(7-azabenzotriazol-1-yl)-N,N,N′,N′-tetramethyluronium |
| | hexafluorophosphate |
| Xphos | 2-Dicyclohexylphosphino-2′,4′,6′-triisopropylbiphenyl |
| TBTU | O-(Benzotriazol-1-yl)-N,N,N′,N′-tetramethyluronium |
| | tetrafluoroborate |
| PyBOP | Benzotriazol-1-yl-oxytripyrrolidinophosphonium |
| | hexafluorophosphate |
| KP-Sil | ready to use silica gel column |
| Xantphos | 4,5-Bis(diphenylphosphino)-9,9-dimethylxanthene |

Examples were analyzed and characterized by the following analytical methods to determine characteristic retention time and mass spectrum:

| | |
|---|---|
| System: | Waters Acquity UPLC-MS: Binary Solvent Manager, |
| | Sample Manager/Organizer, Column Manager, PDA, |
| | ELSD, SQD 3001 |
| Column: | Acquity UPLC BEH C18 1.7 50 × 2.1 mnn |
| Solvent: | A1 = H2O + 0.1% HCOOH |
| | A2 = H2O + 0.2% NH3 |
| | B1 = Acetonitrile |
| Gradient: | 0-1.6 min 1-99% B, 1.6-2.0 min 99% B |
| Flow: | 0.8 mL/min |
| Temperature: | 60° C. |
| Injection: | 2.0 μL |
| Detection: | DAD scan range 210-400 nm -> Peak table |
| | ELSD |
| Method: | MS ESI+, ESI− Switch -> diverse scan ranges possible |

### Method 1: UPLC (ACN—HCOOH)

Instrument: Waters Acquity UPLC-MS SQD 3001; column: Acquity UPLC BEH C18 1.7 50×2.1 mm; eluent A: water+0.1% formic acid, eluent B: acetonitril; gradient: 0-1.6 min 1-99% B, 1.6-2.0 min 99% B; flow 0.8 mL/min; temperature: 60° C.; injection: 2 μL; DAD scan: 210-400 nm; ELSD

### Method 2: UPLC (ACN—NH₃)

Instrument: Waters Acquity UPLC-MS SQD 3001; column: Acquity UPLC BEH C18 1.7 50×2.1 mm; eluent A: water+0.2% ammonia, eluent B: acetonitril; gradient: 0-1.6 min 1-99% B, 1.6-2.0 min 99% B; flow 0.8 mL/min; temperature: 60° C.; injection: 2 μL; DAD scan: 210-400 nm; ELSD

### Method 3:

System: Waters autopurification system: Pump 2545, Sample Manager 2767, CFO, DAD 2996, ELSD 2424, SQD; Column: XBrigde C18 5 μm 100×30 mm; Solvent: A=H2O+0.1% Vol. formic acid (99%), B=acetonitrile; Gradient: 0-8 min 10-100% B, 8-10 min 100% B; Flow: 50 mL/min; temperature: room temp.; Solution: Max. 250 mg/max. 2.5 mL DMSO o. DMF; Injection: 1×2.5 mL; Detection: DAD scan range 210-400 nm; MS ESI+, ESI−, scan range 160-1000 m/z.

### Method 4:

System: Waters autopurification system: Pump 2545, Sample Manager 2767, CFO, DAD 2996, ELSD 2424, SQD; Column: XBrigde C18 5 μm 100×30 mm; Solvent: A=H2O+0.1% Vol. ammonia (99%), B=acetonitrile; Gradient: 0-8 min 10-100% B, 8-10 min 100% B; Flow: 50 mL/min; temperature: room temp.; Solution: Max. 250 mg/max. 2.5 mL DMSO o. DMF; Injection: 1×2.5 mL; Detection: DAD scan range 210-400 nm; MS ESI+, ESI−, scan range 160-1000 m/z.

### Method 5:

(prep. HPLC) System: Labomatic, Pump: HD-5000, Fraction Collector: LABOCOL Vario-4000, UV-Detector: Knauer UVD 2.1 S; Column: Chromatorex C18 10 μm 125×30 mm; Solvent: A=water++0.1% Vol. formic acid (99%), B=Acetonitril; Flow: 150 mL/min; temperature: room temperature; Solution: Max. 250 mg/2 mL DMSO; Injektion: 2×2 mL; Detection: UV 218 nm; Software: SCPA PrepCon5. The following gradients for the preparative HPLC were used according the retention times in the analytic UPLC:

| | | | |
|---|---|---|---|
| Gradient 5a: | 0-15 min | 1-25% B | (for Rt (min): 0-0.54) |
| Gradient 5b: | 0-15 min | 10-50% B | (for Rt (min): 0.54-0.80) |
| Gradient 5c: | 0-15 min | 15-55% B | (for Rt (min): 0.80-1.10) |
| Gradient 5d: | 0-15 min | 30-70% B | (for Rt (min): 1.10-1.35) |
| Gradient 5e: | 0-15 min | 40-80% B | (for Rt (min): 1.35-1.42) |
| Gradient 5f: | 0-15 min | 65-100% B | (for Rt (min): 1.42-2.00) |

### Method 6:

Waters autopurification system: Pump 2545, Sample Manager 2767, CFO, DAD 2996, ELSD 2424, SQD; Column: XBrigde C18 5 μm 100×30 mm; Solvent: A=water+0.1% Vol. formic acid (99%), B=acetonitrile; Gradient: 0-8 min 50-90% B, 8-10 min 100% B; Flow: 50 mL/min; temperature: room temp.; Solution: Max. 250 mg/max. 2.5 mL DMSO o. DMF; Injection: 4×0.7 mL; Detection: DAD scan range 210-400 nm; MS ESI+, ESI−, scan range 160-1000 m/z.

### Method 7:

System: Agilent: Prep 1200, 2×Prep Pump, DLA, MWD, Prep FC; Column: Chiralpak IA 5 μm 250×30 mm; Solvent: Methanol/Ethanol 50:50 (v/v); Flow: 40 mL/min; temperature: room temp.; Detection: UV 254 nm

### Method 8:

System: Sepiatec: Prep SFC₁₀₀; Column: Chiralpak IC 5 μm 250×20 mm; Solvent: CO2/Ethanol+0.4% DEA 8/2; Flow: 80 mL/min; temperature: 40° C.; Detection: UV 254 nm

### Method 9:

System: Agilent: Prep 1200, 2×Prep Pump, DLA, MWD, Prep FC; Column: Chiralpak ID 5 μm 250×30 mm; Solvent: Hexan/2-Propanol 70:30 (v/v); Flow: 50 mL/min; temperature: rom temp.; Detection: UV 254 nm

### Method 10:

System: Agilent: Prep 1200, 2×Prep Pump, DLA, MWD, Gilson: Liquid Handler 215; Column: Chiralpak IC 5 μm 250×30 mm; Solvent: ACN/ethanol 90:10 (v/v); Flow: 50 mL/min; temperature: rom temp.; Detection: UV 220 nm

### Method 11:

System: Waters Acquity UPLC-MS: Binary Solvent Manager, Sample Manager/Organizer, Column Manager, PDA, ELSD, SQD 3001; Column: YMC-Triart C18, 50 mm×2.0 mm, 1.9 μm; Solvent: A=H2O+0.1% Vol. formic acid (99%), B=acetonitrile; Gradient: 0-1.6 min 1-99% B, 1.6-2.0 min 99% B; Flow: 0.8 mL/min; temperature: 60° C.; Detection: DAD scan range 210-400 nm→Peak table; method: MS ESI+, ESI− Switch→diverse scan ranges possible

Column chromatography was performed on a Biotage® Isolera™ Spektra Four Flash Purification System.

NMR peak forms of selected examples are stated as they appear in the spectra, possible higher order effects have not been considered. In cases were a signal is very broad or is partially or totally hidden by a solvent peak the total number of hydrogen atoms displayed in NMR spectra can differ from the number of hydrogen atoms present in the respective molecule.

The ¹H-NMR data of selected examples are listed in the form of ¹H-NMR peaklists. For each signal peak the 6 value in ppm is given, followed by the signal intensity, reported in round brackets. The 6 value-signal intensity pairs from different peaks are separated by semicolons. Therefore, a peaklist is described by the general form: δ₁ (intensity₁); δ₂ (intensity₂); . . . ; δᵢ (intensity₁); . . . ; δₙ (intensityₙ).

The intensity of a sharp signal correlates with the height (in cm) of the signal in a printed NMR spectrum. When compared with other signals, this data can be correlated to the real ratios of the signal intensities. In the case of broad signals, more than one peak, or the center of the signal along with their relative intensity, compared to the most intense signal displayed in the spectrum, are shown. A ¹H-NMR peaklist is similar to a classical ¹H-NMR readout, and thus usually contains all the peaks listed in a classical NMR interpretation. Moreover, similar to classical ¹H-NMR printouts, peaklists can show solvent signals, signals derived from stereoisomers of target compounds (also the subject of the invention), and/or peaks of impurities. The peaks of stereoisomers, and/or peaks of impurities are typically displayed with a lower intensity compared to the peaks of the target compounds (e.g., with a purity of >90%). Such stereoisomers and/or impurities may be typical for the particular manufacturing process, and therefore their peaks may help to identify the reproduction of our manufacturing process on the basis of “by-product fingerprints”. An expert who calculates the peaks of the target compounds by known methods (MestReC, ACD simulation, or by use of empirically evaluated expectation values), can isolate the peaks of target compounds as required, optionally using additional intensity filters. Such an operation would be similar to peak-picking in classical ¹H-NMR interpretation. A detailed description of the reporting of NMR data in the form of peaklists can be found in the publication “Citation of NMR Peaklist Data within Patent Applications” (cf. Research Disclosure Database Number 605005, 2014, 1 Aug. 2014, or http://www.researchdisclosure.com/searching-disclosures).

Yields in % reflect the purity of the desired product obtained if not stated otherwise; purities significantly below 90% were specified explicity if appropriate.

If not stated otherwise, starting materials as mentioned in the protocols were purchased from commercial suppliers.

The IUPAC names of the examples and intermediates were generated using the program ‘ACD/Name batch version 12.01’ from ACD LABS, and were adapted if needed.

### Intermediates

### Intermediate 1A

### 6-bromo-2-(trifluoromethyl)quinoline-4-carboxylic acid

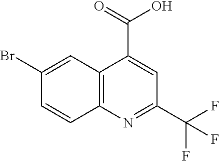 [SMILES: O=C(O)c1cc(C(F)(F)F)nc2ccc(Br)cc12]

300 mg (1.33 mmol) of 5-bromo-1H-indole-2,3-dione was suspended in 3 mL water in a microwave vial. 82 mg (1.46 mmol) potassium hydroxide, 152 μL (2.65 mmol) acetic acid and 152 mg (1.86 mmol) sodium acetate were added so that the pH was around 5. The solution was cooled to 10° C. and 238 μL (2.65 mmol) 1,1,1-trifluoroacetone was added rapidly, the microwave vial was sealed and heated in the microwave for 2 h at 120° C. The reaction was stopped by the addition of 10% aqueous hydrochloric acid solution and the resulting precipitate was isolated by filtration, washed with water and dried in a vacuum drying cabinet at 50° C. overnight to obtain 409 mg (1.28 mmol, 96%) of the desired title compound.

1H NMR (300 MHz, DMSO do): δ (ppm)=8.14 (dd, 1H), 8.21 (d, 1H), 8.32 (s, 1H), 9.09 (d, 1H), 14.50 (br. s., 1H).

### Intermediate 2A

### 6-bromo-2-carbamoylquinoline-4-carboxylic acid

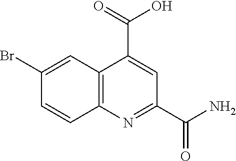 [SMILES: NC(=O)c1cc(C(=O)O)c2cc(Br)ccc2n1]

### Step 1: 6-Bromoquinoline-2,4-dicarboxylic acid

To a mixture of 1.5 g (6.64 mmol) 5-bromo-1H-indole-2,3-dione in hot 15 mL of 33% aq. potassium hydroxide solution was added 1.02 g (11.6 mmol) pyruvic acid and this mixture was heated at 40° C. for 16 hours. To the formed thicky paste 50 mL of 33% aq. potassium hydroxide solution was added and stirred. The solid was isolated by filtration and washed with 33% aq. potassium hydroxide solution and ethanol. The solid was then diluted in water and 10% aq. sulfuric acid was added (pH below 7). The formed solid was isolated by filtration and dried for 8 hours in vacuum. The solid was the desired 6-bromoquinoline-2,4-dicarboxylic acid, which was used without further purification. Yield: 1.5 g (74%)

¹H-NMR (300 MHz, DMSO d₆) δ (ppm)=8.06 (dd, 1H), 8.18 (d, 1H), 8.52 (s, 1H), 9.08 (d, 1H).

### Step 2: dimethyl 6-bromoquinoline-2,4-dicarboxylate

A mixture of 1.5 g (5.07 mmol) of the diacid of step 1) intermediate 2A) and 3.7 mL (50.7 mmol) thionyl chloride was heated at 80° C. for 16 hours. After cooling to 25° C. the resulting suspension was evaporated to dryness in vacuum. This crude product was suspended in 10 mL methanol and refluxed for 3 hours. After cooling to 25° C. the formed solid was isolated by filtration. To the filtrate water was added and the additional formed solid was isolated by filtration. The combined crude products were purified via a Biotage chromatography system (25 g snap KP-Sil column, hexane/0-100% ethylacetate then ethyl acetate/0-10% methanol). Using this methodology we obtained the desired dimethyl 6-bromoquinoline-2,4-dicarboxylate. Yield: 180 mg (10%)

¹H-NMR (300 MHz, DMSO d₆) δ (ppm)=3.98 (s, 3H), 4.02 (s, 3H), 8.10 (dd, 1H), 8.21 (d, 1H), 8.51 (s, 1H), 8.97 (d, 1H).

### Step 3: methyl 6-bromo-2-carbamoylquinoline-4-carboxylate

To a solution of 180 mg (0.56 mmol) diester of step 2) intermediate 2A) in 2.0 mL methanol was added 1.19 mL of a 7M solution of ammonia in methanol and stirred for 1 hour at 50° C. Then additional 15 equiv. of ammonia was added and stirring was continued for 2 hours at 50° C. After cooling to 25° C. the formed solid was isolated by filtration and dried. Using this methodology we obtained the desired methyl 6-bromo-2-carbamoylquinoline-4-carboxylate. Yield: 120 mg (66%)

¹H-NMR (300 MHz, DMSO d₆) δ (ppm)=4.01 (s, 3H), 7.95 (br. s., 1H), 8.04-8.18 (m, 2H), 8.41 (br. s., 1H), 8.57 (s, 1H), 8.97 (d, 1H).

### Step 4: 6-bromo-2-carbamoylquinoline-4-carboxylic acid

To a solution of 120 mg (0.39 mmol) of the compound from step 3) intermediate 2A) in 1.79 mL methanol was added a solution of 279 mg sodium hydroxide in 3.58 mL water. This mixture was stirred for 2 hours at 25° C. and then concentrated in vacuum. The residue was diluted with water and 10% aq. sulfuric acid was added up to pH 2. After stirring for additional 15 minutes the formed solid was isolated by filtration and dried in vacuum. Using this methodology we obtained the desired title compound. Yield: 106 mg (74%)

¹H-NMR (300 MHz, DMSO d₆) δ (ppm)=7.89 (br. s., 1H), 8.03 (dt, 1H), 8.07-8.18 (m, 1H), 8.36 (br. s., 1H), 8.43-8.55 (m, 1H), 9.10 (dd, 1H).

### Intermediate 3A

### 2-carbamoyl-6,7-difluoroquinoline-4-carboxylic acid

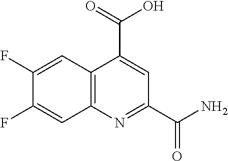 [SMILES: NC(=O)c1cc(C(=O)O)c2cc(F)c(F)cc2n1]

### Step 1: 6,7-difluoroquinoline-2,4-dicarboxylic acid

In analogy to step 1) of intermediate 2A) 1.5 g (8.19 mmol) 5,6-difluoro-1H-indole-2,3-dione (see e.g. Journal of Organic Chemistry, 1958, 1858) were reacted to give 1.04 g (48%) 6,7-difluoroquinoline-2,4-dicarboxylic acid.

¹H-NMR (300 MHz, DMSO d) δ (ppm)=8.30 (dd, 1H), 8.52 (s, 1H), 8.79 (dd, 1H).

### Step 2: dimethyl 6,7-difluoroquinoline-2,4-dicarboxylate

In analogy to step 2) of intermediate 2A) 1.04 g (4.11 mmol) 6,7-difluoroquinoline-2,4-dicarboxylic acid of step 1) of intermediate 3A) were reacted to give 640 mg (52%) dimethyl 6,7-difluoroquinoline-2,4-dicarboxylate.

¹H-NMR (300 MHz, DMSO d₆) δ (ppm)=3.98 (s, 3H), 4.01 (s, 3H), 8.37 (dd, 1H), 8.50 (s, 1H), 8.69 (dd, 1H).

### Step 3: methyl 2-carbamoyl-6,7-difluoroquinoline-4-carboxylate

In analogy to step 3) of intermediate 2A) 340 mg (1.21 mmol) dimethyl 6,7-difluoroquinoline-2,4-dicarboxylate of step 2) of intermediate 3A) were reacted to give 180 mg (53%) methyl 2-carbamoyl-6,7-difluoroquinoline-4-carboxylate.

¹H-NMR (400 MHz, DMSO d₆) δ (ppm)=1.16 (t, 1H), 3.99-4.04 (m, 3H), 7.95 (br. s., 1H), 8.16 (dd, 1H), 8.33 (br. s., 1H), 8.56 (s, 1H), 8.70 (dd, 1H).

### Step 4: 2-carbamoyl-6,7-difluoroquinoline-4-carboxylic acid

In analogy to step 4) of intermediate 2A) 173 mg (0.65 mmol) methyl 2-carbamoyl-6,7-difluoroquinoline-4-carboxylate of step 3) of intermediate 3A) were reacted to give 86 mg (52%) of the desired title compound.

¹H-NMR (300 MHz, DMSO d₆) δ (ppm)=7.95 (br. s., 1H), 8.15 (dd, 1H), 8.34 (br. s., 1H), 8.57 (s, 1H), 8.83 (d, 1H), 14.15 (br. s., 1H).

### Intermediate 4A

### 2-carbamoylquinoline-4-carboxylic acid

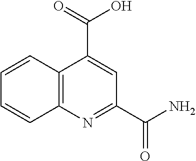 [SMILES: NC(=O)c1cc(C(=O)O)c2ccccc2n1]

### Step 1: dimethyl quinoline-2,4-dicarboxylate

In analogy to step 2) of intermediate 2A) 11.4 g (44.9 mmol) commercially available quinoline-2,4-dicarboxylic acid were reacted to give 6.44 g (59%) dimethyl quinoline-2,4-dicarboxylate.

¹H-NMR (300 MHz, DMSO d₆) δ (ppm)=3.98 (s, 3H), 4.01 (s, 3H), 7.88 (ddd, 1H), 7.96 (ddd, 1H), 8.26 (dd, 1H), 8.46 (s, 1H), 8.70 (dd, 1H).

### Step 2: methyl 2-carbamoylquinoline-4-carboxylate

In analogy to step 3) of intermediate 2A) 1.0 g (4.08 mmol) dimethyl quinoline-2,4-dicarboxylate of step 1) of intermediate 4A) were reacted to give 650 mg (66%) methyl 2-carbamoylquinoline-4-carboxylate.

¹H-NMR (400 MHz, DMSO d₆) δ (ppm)=4.01 (s, 3H), 7.85 (ddd, 1H), 7.89 (br. s., 1H), 7.95 (ddd, 1H), 8.22 (d, 1H), 8.37 (br. s., 1H), 8.53 (s, 1H), 8.71 (d, 1H).

### Step 3: 2-carbamoylquinoline-4-carboxylic acid

In analogy to step 4) of intermediate 2A) 650 mg (2.82 mmol) methyl 2-carbamoylquinoline-4-carboxylate of step 2) of intermediate 4A) were reacted to give 540 mg (86%) of the desired title compound.

¹H-NMR (400 MHz, DMSO d₆) δ (ppm)=7.82 (dt, 1H), 7.86 (br. s., 1H), 7.92 (td, 1H), 8.20 (d, 1H), 8.34 (br. s., 1H), 8.50 (s, 1H), 8.78 (d, 1H), 13.98 (br. s., 1H).

### Intermediate 5A

### 2-carbamoyl-6,8-dichloroquinoline-4-carboxylic acid

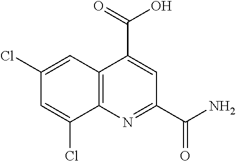 [SMILES: NC(=O)c1cc(C(=O)O)c2cc(Cl)cc(Cl)c2n1]

### Step 1: 6,8-dichloroquinoline-2,4-dicarboxylic acid

In analogy to step 1) of intermediate 2A) 1.5 g (6.94 mmol) commercially available 5,7-dichloro-1H-indole-2,3-dione were reacted to give 350 mg (17%) 6,8-dichloroquinoline-2,4-dicarboxylic acid.

1H-NMR (300 MHz, DMSO d6) δ (ppm)=8.29 (d, 1H), 8.58 (s, 1H), 8.88 (d, 1H), 13.99 (br. s., 1H).

### Step 2: dimethyl 6,8-dichloroquinoline-2,4-dicarboxylate

In analogy to step 2) of intermediate 2A) 400 mg (1.24 mmol) 6,8-dichloroquinoline-2,4-dicarboxylic acid of step 1) of intermediate 5A) were reacted to give 410 mg (83%) dimethyl 6,8-dichloroquinoline-2,4-dicarboxylate.

1H-NMR (300 MHz, DMSO d6) δ (ppm)=4.00 (s, 3H), 4.02 (s, 3H), 8.34 (d, 1H), 8.58 (s, 1H), 8.76 (d, 1H).

### Step 3: methyl 2-carbamoyl-6,8-dichloroquinoline-4-carboxylate

In analogy to step 3) of intermediate 2A) 310 mg (0.99 mmol) dimethyl 6,8-dichloroquinoline-2,4-dicarboxylate of step 2) of intermediate 5A) were reacted to give 140 mg (45%) methyl 2-carbamoyl-6,8-dichloroquinoline-4-carboxylate.

1H-NMR (400 MHz, DMSO d6) δ (ppm)=4.01 (s, 3H), 8.08 (s, 2H), 8.29 (d, 1H), 8.62 (s, 1H), 8.74 (d, 1H).

### Step 4: 2-carbamoyl-6,8-dichloroquinoline-4-carboxylic acid

In analogy to step 4) of intermediate 2A) 140 mg (0.47 mmol) methyl 2-carbamoyl-6,8-dichloroquinoline-4-carboxylate of step 3) of intermediate 5A) were reacted to give 145 mg (98%) of the desired title compound.

1H-NMR (400 MHz, DMSO d6) δ (ppm)=7.94 (br. s., 1H), 8.00 (br. s., 1H), 8.14 (d, 1H), 8.41 (s, 1H), 8.95 (d, 1H).

### Intermediate 6A

### 2-(methylcarbamoyl)quinoline-4-carboxylic acid

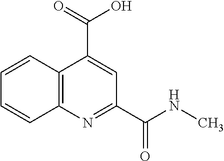 [SMILES: CNC(=O)c1cc(C(=O)O)c2ccccc2n1]

### Step 1: methyl 2-(methylcarbamoyl)quinoline-4-carboxylate

To a solution of 250 mg (1.02 mmol) dimethyl quinoline-2,4-dicarboxylate of step 1) intermediate 4A) in 2.5 mL methanol was added 5.1 mL of a 2M solution of methyl amine in THF and stirred for 1 hour at 50° C. After cooling to 25° C. Isolute® was added to the reaction mixture and then evaporated to dryness. The absorbed material was then purified using a Biotage chromatography system (25 g snap KP-Sil column, hexane/0-100% ethyl acetate, then ethyl acetate/0-75% methanol). Using this methodology we got the desired methyl 2-(methylcarbamoyl)quinoline-4-carboxylate. Yield: 156 mg (61%)

1H-NMR (300 MHz, DMSO d6) δ (ppm)=2.90 (d, 3H), 4.01 (s, 3H), 7.80-7.88 (m, 1H), 7.95 (ddd, 1H), 8.21 (d, 1H), 8.51 (s, 1H), 8.70 (dd, 1H), 9.00 (q, 1H).

### Step 2: 2-(methylcarbamoyl)quinoline-4-carboxylic acid

In analogy to step 4) of intermediate 2A) 156 mg (0.64 mmol) methyl 2-(methylcarbamoyl)quinoline-4-carboxylate of step 2) of intermediate 6A) were reacted to give 138 mg (91%) of the desired title compound.

1H-NMR (300 MHz, DMSO d6) δ (ppm)=2.90 (d, 3H), 7.78-7.86 (m, 1H), 7.93 (td, 1H), 8.20 (d, 1H), 8.50 (s, 1H), 8.78 (d, 1H), 8.98 (q, 1H), 14.03 (br. s., 1H).

### Intermediate 7A

### 2-(dimethylcarbamoyl)quinoline-4-carboxylic acid

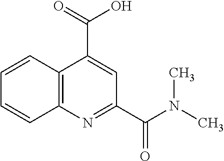 [SMILES: CN(C)C(=O)c1cc(C(=O)O)c2ccccc2n1]

### Step 1: methyl 2-(dimethylcarbamoyl)quinoline-4-carboxylate

To a solution of 500 mg (2.04 mmol) dimethyl quinoline-2,4-dicarboxylate of step 1) intermediate 4A) in 5.0 mL methanol was added 10.2 mL of a 2M solution of dimethyl amine in methanol and stirred for 1 hour at 50° C. Then additional 15 mL of a 2M solution of dimethyl amine in methanol was added and stirring was continued for 16 hours. After cooling to 25° C. Isolute® was added to the reaction mixture and then evaporated to dryness. The absorbed material was then purified using a Biotage chromatography system (25 g snap KP-Sil column, hexane/0-100% ethyl acetate, then ethyl acetate/0-75% methanol). Using this methodology we got the desired methyl 2-(dimethylcarbamoyl)quinoline-4-carboxylate. Yield: 140 mg (25%)

1H-NMR (400 MHz, DMSO d6) δ (ppm)=3.06 (s, 3H), 3.09 (s, 3H), 4.01 (s, 3H), 7.78-7.86 (m, 1H), 7.92 (ddd, 1H), 8.07 (s, 1H), 8.15 (d, 1H), 8.66 (d, 1H).

### Step 2: 2-(dimethylcarbamoyl)quinoline-4-carboxylic acid

In analogy to step 4) of intermediate 2A) 140 mg (0.54 mmol) methyl 2-(dimethylcarbamoyl)quinoline-4-carboxylate of step 2) of intermediate 7A) were reacted to give 68 mg (45%) of the desired title compound.

1H-NMR (300 MHz, DMSO d6) δ (ppm)=3.03 (s, 3H), 3.07 (s, 3H), 7.74-7.82 (m, 1H), 7.85-7.92 (m, 1H), 8.01 (s, 1H), 8.12 (d, 1H), 8.72 (d, 1H), 14.03 (br. s., 1H).

### Intermediate 8A

### 6-fluoro-2-carbamoylquinoline-4-carboxylic acid

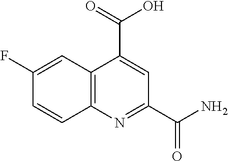 [SMILES: NC(=O)c1cc(C(=O)O)c2cc(F)ccc2n1]

### Step 1: 6-fluoroquinoline-2,4-dicarboxylic acid

In analogy to step 1) of intermediate 2A) 2.0 g (12.1 mmol) commercially available 5-fluoro-1H-indole-2,3-dione were reacted to give 1.86 g (63%) 6-fluoroquinoline-2,4-dicarboxylic acid.

¹H-NMR (300 MHz, DMSO d₆) δ (ppm)=7.83-7.92 (m, 1H), 8.33 (dd, 1H), 8.53-8.62 (m, 2H), 13.86 (br. s., 1H).

### Step 2: dimethyl 6-fluoroquinoline-2,4-dicarboxylate

In analogy to step 2) of intermediate 2A) 1.86 g (7.91 mmol) 6-fluoroquinoline-2,4-dicarboxylic acid of step 1) of intermediate 8A) were reacted to give 1.51 g (69%) dimethyl 6-fluoroquinoline-2,4-dicarboxylate.

¹H-NMR (300 MHz, DMSO d₆) δ (ppm)=3.98 (s, 3H), 4.01 (s, 3H), 7.91 (ddd, 1H), 8.37 (dd, 1H), 8.48 (dd, 1H), 8.54 (s, 1H).

### Step 3: methyl 2-carbamoyl-6-fluoroquinoline-4-carboxylate

In analogy to step 3) of intermediate 2A) 310 mg (1.18 mmol) dimethyl 6-fluoroquinoline-2,4-dicarboxylate of step 2) of intermediate 8A) were reacted to give 210 mg (68%) methyl 2-carbamoyl-6-fluoroquinoline-4-carboxylate.

¹H-NMR (300 MHz, DMSO d₆) δ (ppm)=4.01 (s, 3H), 7.84-7.95 (m, 2H), 8.28 (dd, 1H), 8.37 (br. s., 1H), 8.48 (dd, 1H), 8.59 (s, 1H).

### Step 4: 6-fluoro-2-carbamoylquinoline-4-carboxylic acid

In analogy to step 4) of intermediate 2A) 210 mg (0.85 mmol) methyl 2-carbamoyl-6-fluoroquinoline-4-carboxylate of step 3) of intermediate 8A) were reacted to give 156 mg (79%) of the desired title compound.

¹H-NMR (400 MHz, DMSO d₆) δ (ppm)=7.81-7.90 (m, 2H), 8.26 (dd, 1H), 8.32-8.37 (m, 1H), 8.55-8.62 (m, 2H), 13.75 (br. s., 1H).

### Intermediate 9A

### 6-bromo-7-fluoro-2-(trifluoromethyl)quinoline-4-carboxylic acid

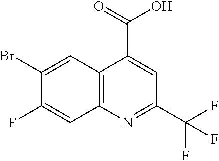 [SMILES: O=C(O)c1cc(C(F)(F)F)nc2cc(F)c(Br)cc12]

In analogy to intermediate 1A) 1.0 g (4.02 mmol) commercially available 5-bromo-6-fluoro-1H-indole-2,3-dione were reacted to give using conventional heating at 105° C. for 3 hours 990 mg (59%) of the desired title compound.

¹H-NMR (300 MHz, DMSO d₆) δ (ppm)=8.24-8.32 (m, 2H), 9.25 (d, 1H), 14.57 (br. s., 1H).

### Intermediate 10A

### 6-chloro-7-fluoro-2-(trifluoromethyl)quinoline-4-carboxylic acid

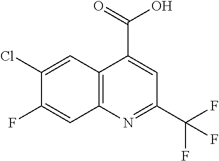 [SMILES: O=C(O)c1cc(C(F)(F)F)nc2cc(F)c(Cl)cc12]

In analogy to intermediate 1A) 1.0 g (4.91 mmol) commercially available 5-chloro-6-fluoro-1H-indole-2,3-dione were reacted to give using conventional heating at 105° C. for 3 hours 1.02 g (58%) of the desired title compound.

¹H-NMR (300 MHz, DMSO d₆) δ (ppm)=8.31 (s, 1H), 8.34 (d, 1H), 9.09 (d, 1H), 11.27 (s, 1H).

### Intermediate 11A

### 6,8-dichloro-2-(trifluoromethyl)quinoline-4-carboxylic acid

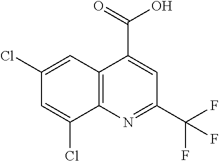 [SMILES: O=C(O)c1cc(C(F)(F)F)nc2c(Cl)cc(Cl)cc12]

In analogy to intermediate 1A, 1 g (4.63 mmol) 5,7-dichloro-1H-indole-2,3-dione was heated with 830 μL (9.26 mmol) 1,1,1-trifluoroacetone, 286 mg (5.10 mmol) potassium hydroxide, 530 μL (9.26 mmol) acetic acid and 531 mg (6.48 mmol) sodium acetate in 10 mL water for 2 h at 120° C. in the microwave to obtain 1.40 g (4.52 mmol, 98%) of the desired title compound after aqueous work-up.

1H NMR (300 MHz, DMSO d₆): δ (ppm)=8.39 (d, 1H), 8.42 (s, 1H), 8.87 (d, 1H).

### Intermediate 12A

### 6-chloro-2-(trifluoromethyl)quinoline-4-carboxylic acid

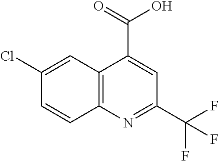 [SMILES: O=C(O)c1cc(C(F)(F)F)nc2ccc(Cl)cc12]

In analogy to intermediate 1A, 500 mg (2.75 mmol) 5-chloro-1H-indole-2,3-dione was heated with 494 μL (9.26 mmol) 1,1,1-trifluoroacetone, 170 mg (3.03 mmol) potassium hydroxide, 315 μL (5.51 mmol) acetic acid and 316 mg (3.86 mmol) sodium acetate in 5 mL water for 2 h at 120° C. in the microwave to obtain 726 mg (2.63 mmol, 96%) of the desired title compound after aqueous work-up.

1H NMR (300 MHz, DMSO d₆): δ (ppm)=8.03 (dd, 1H), 8.30 (d, 1H), 8.33 (s, 1H), 8.92 (d, 1H).

### Intermediate 13A

### 8-bromo-2-(trifluoromethyl)quinoline-4-carboxylic acid

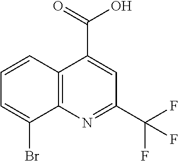 [SMILES: O=C(O)c1cc(C(F)(F)F)nc2c(Br)cccc12]

In analogy to intermediate 1A, 300 mg (1.33 mmol) 7-bromo-1H-indole-2,3-dione was heated with 238 μL (2.65 mmol) 1,1,1-trifluoroacetone, 81 mg (1.46 mmol) potassium hydroxide, 152 μL (2.65 mmol) acetic acid and 152 mg (1.86 mmol) sodium acetate in 3 mL water for 2 h at 120° C. in the microwave to obtain 373 mg (1.17 mmol, 88%) of the desired title compound after aqueous work-up.

1H NMR (300 MHz, DMSO d₆): δ (ppm)=7.81 (dd, 1H), 8.35 (s, 1H), 8.40 (dd, 1H), 8.78 (dd, 1H), 14.56 (br. s., 1H).

### Intermediate 14A

### 8-bromo-6-methyl-2-(trifluoromethyl)quinoline-4-carboxylic acid

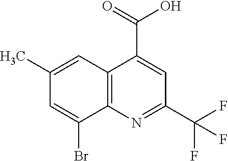 [SMILES: Cc1cc(Br)c2nc(C(F)(F)F)cc(C(=O)O)c2c1]

In analogy to intermediate 1A, 300 mg (1.25 mmol) 7-bromo-5-methyl-1H-indole-2,3-dione was heated with 224 μL (2.50 mmol) 1,1,1-trifluoroacetone, 77 mg (1.37 mmol) potassium hydroxide, 143 μL (2.50 mmol) acetic acid and 144 mg (1.75 mmol) sodium acetate in 3 mL water for 2 h at 120° C. in the microwave to obtain 352 mg (1.05 mmol, 84%) of the desired title compound after aqueous work-up.

1H NMR (300 MHz, DMSO d₆): δ (ppm)=2.58 (s, 3H), 8.25-8.32 (m, 2H), 8.53 (s, 1H), 14.49 (br. s., 1H).

### Intermediate 15A

### 5,6-dichloro-2-(trifluoromethyl)quinoline-4-carboxylic acid and 6,7-dichloro-2-(trifluoromethyl)quinoline-4-carboxylic acid

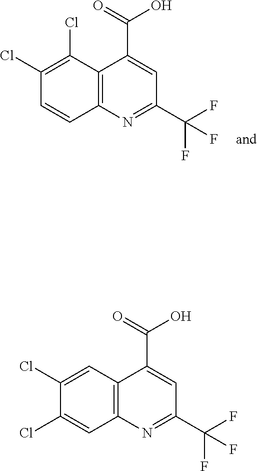 [SMILES: O=C(O)c1cc(C(F)(F)F)nc2ccc(Cl)c(Cl)c12 ; O=C(O)c1cc(C(F)(F)F)nc2cc(Cl)c(Cl)cc12]

In analogy to intermediate 1A, 300 mg (1.39 mmol) of a mixture of 4,5-dichloro-1H-indole-2,3-dione and 5,6-dichloro-1H-indole-2,3-dione (3:1) was heated with 249 μL (2.78 mmol) 1,1,1-trifluoroacetone, 86 mg (1.53 mmol) potassium hydroxide, 159 μL (2.78 mmol) acetic acid and 159 mg (1.94 mmol) sodium acetate in 3 mL water for 4 h at 120° C. in the microwave to obtain 361 mg (1.16 mmol, 84%) of the desired title compound mixture (3:1) after aqueous work-up.

### 5,6-dichloro-2-(trifluoromethyl)quinoline-4-carboxylic acid

Method 1: Rₜ=1.07 min

MS (ESIpos): m/z=310 (M+H)⁺

### 6,7-dichloro-2-(trifluoromethyl)quinoline-4-carboxylic acid

Method 1: Rₜ=1.34 min

MS (ESIpos): m/z=310 (M+H)⁺

### Intermediate 16A

### 5-fluoro-2-(trifluoromethyl)quinoline-4-carboxylic acid

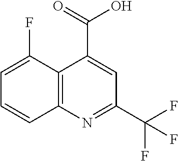 [SMILES: O=C(O)c1cc(C(F)(F)F)nc2cccc(F)c12]

In analogy to intermediate 1A, 300 mg (1.81 mmol) 4-fluoro-1H-indole-2,3-dione was heated with 326 μL (3.63 mmol) 1,1,1-trifluoroacetone, 112 mg (1.99 mmol) potassium hydroxide, 208 μL (3.63 mmol) acetic acid and 209 mg (2.54 mmol) sodium acetate in 3 mL water for 2 h at 100° C. in the microwave to obtain 315 mg (1.22 mmol, 67%) of the desired title compound after aqueous work-up.

1H NMR (400 MHz, DMSO d₆): δ (ppm)=7.83-7.92 (m, 1H), 8.09 (dd, 1H), 8.25 (s, 1H), 8.90 (dd, 1H), 14.50 (br. s., 1H).

### Intermediate 17A

### 7-fluoro-2-(trifluoromethyl)quinoline-4-carboxylic acid

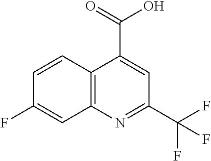 [SMILES: O=C(O)c1cc(C(F)(F)F)nc2cc(F)ccc12]

In analogy to intermediate 1A, 200 mg (1.21 mmol) 6-fluoro-1H-indole-2,3-dione was heated with 217 μL (2.42 mmol) 1,1,1-trifluoroacetone, 75 mg (1.33 mmol) potassium hydroxide, 139 μL (2.42 mmol) acetic acid and 139 mg (1.70 mmol) sodium acetate in 2 mL water for 2 h at 80° C. in the microwave. As the conversion was not complete further 217 μL (2.42 mmol) 1,1,1-trifluoroacetone was added to the reaction mixture and heated again for 1 h at 80° C. in the microwave. Once again 217 μL (2.42 mmol) 1,1,1-trifluoroacetone was added and the reaction mixture was heated a third time for 1 h at 100° C. in the microwave to obtain 227 mg (0.88 mmol, 72%) of the desired title compound after aqueous work-up.

1H NMR (300 MHz, DMSO d₆): δ (ppm)=7.83-7.93 (m, 1H), 8.10 (dd, 1H), 8.26 (s, 1H), 8.90 (dd, 1H), 14.51 (br. s., 1H).

### Intermediate 18A

### 6-fluoro-2-(trifluoromethyl)quinoline-4-carboxylic acid

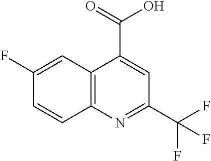 [SMILES: O=C(O)c1cc(C(F)(F)F)nc2ccc(F)cc12]

In analogy to intermediate 1A, 200 mg (1.21 mmol) 6-fluoro-1H-indole-2,3-dione was heated with 217 μL (2.42 mmol) 1,1,1-trifluoroacetone, 75 mg (1.33 mmol) potassium hydroxide, 139 μL (2.42 mmol) acetic acid and 139 mg (1.70 mmol) sodium acetate in 2 mL water for 2 h at 80° C. in the microwave to obtain 219 mg (0.85 mmol, 70%) of the desired title compound after aqueous work-up.

1H NMR (400 MHz, DMSO d₆): δ (ppm)=7.96 (ddd, 1H), 8.34 (s, 1H), 8.38 (dd, 1H), 8.59 (dd, 1H), 14.47 (br. s., 1H).

### Intermediate 19A

### 6,7-difluoro-2-(trifluoromethyl)quinoline-4-carboxylic acid

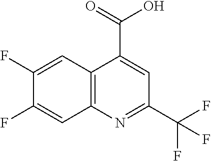 [SMILES: O=C(O)c1cc(C(F)(F)F)nc2cc(F)c(F)cc12]

In analogy to intermediate 1A, 265 mg (1.45 mmol) 5,6-difluoro-1H-indole-2,3-dione was heated with 259 μL (2.89 mmol) 1,1,1-trifluoroacetone, 89 mg (1.59 mmol) potassium hydroxide, 166 μL (2.89 mmol) acetic acid and 166 mg (2.03 mmol) sodium acetate in 2.7 mL water for 1 h at 120° C. in the microwave. As the conversion was not complete further 259 μL (2.89 mmol) 1,1,1-trifluoroacetone was added to the reaction mixture and heated again for 1 h at 120° C. in the microwave to obtain 312 mg (1.13 mmol, 78%) of the desired title compound after aqueous work-up.

1H NMR (300 MHz, DMSO d₆): δ (ppm)=8.33 (s, 1H), 8.41 (dd, 1H), 8.81 (dd, 1H), 14.62 (br. s., 1H).

### Intermediate 20A

### 2-(trifluoromethyl)quinoline-4-carboxylic acid

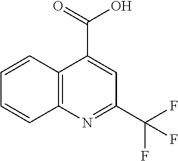 [SMILES: O=C(O)c1cc(C(F)(F)F)nc2ccccc12]

In analogy to intermediate 1A) 3.0 g (20.4 mmol) commercially available 1H-indole-2,3-dione were reacted to give 4.68 g (92%) of the desired title compound.

¹H-NMR (400 MHz, DMSO d) δ (ppm)=7.88-7.93 (m, 1H), 8.00 (ddd, 1H), 8.24 (s, 1H), 8.26 (d, 1H), 8.78 (dd, 1H), 14.39 (br. s., 1H).

### Intermediate 21A

### 6-(trifluoromethoxy)-2-(trifluoromethyl)quinoline-4-carboxylic acid

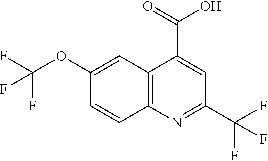 [SMILES: O=C(O)c1cc(C(F)(F)F)nc2ccc(OC(F)(F)F)cc12]

In analogy to intermediate 1A, 200 mg (0.87 mmol) 5-(trifluoromethoxy)-1H-indole-2,3-dione was heated with 388 μL (4.33 mmol) 1,1,1-trifluoroacetone, 53 mg (0.95 mmol) potassium hydroxide, 99 μL (1.73 mmol) acetic acid and 99 mg (1.21 mmol) sodium acetate in 2 mL water for 1 h at 120° C. in the microwave to obtain 269 mg (0.83 mmol, 96%) of the desired title compound after aqueous work-up.

1H NMR (400 MHz, DMSO d₆): δ (ppm)=8.01 (dd, 1H), 8.38 (s, 1H), 8.44 (d, 1H), 8.85-8.89 (m, 1H), 14.57 (br. s., 1H).

### Intermediate 22A

### 7-bromo-2-(trifluoromethyl)quinoline-4-carboxylic acid

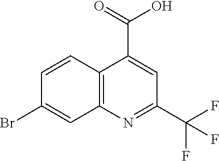 [SMILES: O=C(O)c1cc(C(F)(F)F)nc2cc(Br)ccc12]

In analogy to intermediate 1A, 300 mg (1.33 mmol) 6-bromo-1H-indole-2,3-dione was heated with 238 μL (2.65 mmol) 1,1,1-trifluoroacetone, 82 mg (1.46 mmol) potassium hydroxide, 152 μL (2.65 mmol) acetic acid and 152 mg (1.86 mmol) sodium acetate in 3 mL water for 2 h at 120° C. in the microwave to obtain 361 mg (1.13 mmol, 85%) of the desired title compound after aqueous work-up.

1H NMR (400 MHz, DMSO d): δ (ppm)=8.07 (dd, 1H), 8.30 (s, 1H), 8.54 (d, 1H), 8.76 (d, 1H), 14.49 (br. s., 1H).

### Intermediate 23A

### 2,6-bis(trifluoromethyl)quinoline-4-carboxylic acid

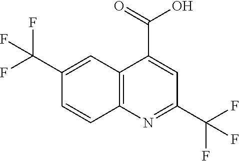 [SMILES: O=C(O)c1cc(C(F)(F)F)nc2ccc(C(F)(F)F)cc12]

In analogy to intermediate 1A, 300 mg (1.39 mmol) 5-(trifluoromethyl)-1H-indole-2,3-dione was heated with 250 μL (2.79 mmol) 1,1,1-trifluoroacetone, 86 mg (1.53 mmol) potassium hydroxide, 160 μL (2.79 mmol) acetic acid and 160 mg (1.95 mmol) sodium acetate in 3 mL water for 1 h at 120° C. in the microwave. As the conversion was not complete further 250 μL (2.79 mmol) 1,1,1-trifluoroacetone was added to the reaction mixture and heated again for 1 h at 120° C. in the microwave to obtain 302 mg (0.78 mmol, 56%) of the desired title compound after aqueous work-up.

1H NMR (300 MHz, DMSO d): δ (ppm)=8.27 (dd, 1H), 8.43 (s, 1H), 8.50 (d, 1H), 9.31 (s, 1H).

### Intermediate 24A

### 6-chloro-2-cyclopropylquinoline-4-carboxylic acid

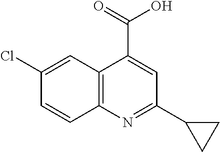 [SMILES: O=C(O)c1cc(C2CC2)nc2ccc(Cl)cc12]

In analogy to intermediate 1A) we got from 5.0 g (27.5 mmol) commercially available 5-chloro-1H-indole-2,3-dione and 4.63 g (55.1 mmol) of 1-cyclopropytethanone instead of 1,1,1-trifluoroacetone and using conventional heating at 105° C. for 5 hours a solid and a filtrate in which the desired title compound was included. Both parts were combined and 2N aq. sodium hydroxide solution was added up to pH of 10, and then extracted three times with 30 mL ethyl acetate. The aqueous phase was then acidified with 10% aq. sulfuric acid up to pH of 3. The formed solid was isolated by filtration and dried. Using this methodology we obtained the desired title compound. Yield: 150 mg (2.1%)

¹H-NMR (300 MHz, DMSO d₆) δ (ppm)=1.06-1.12 (m, 4H), 2.40 (t, 1H), 7.74 (dd, 1H), 7.87-7.96 (m, 2H), 8.71 (d, 1H), 13.83 (s, 1H).

### Intermediate 25A

### 2-cyclopropyl-5-fluoroquinoline-4-carboxylic acid

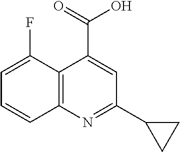 [SMILES: O=C(O)c1cc(C2CC2)nc2cccc(F)c12]

In analogy to intermediate 1A, 300 mg (1.82 mmol) 4-fluoro-1H-indole-2,3-dione was heated with 900 μL (9.08 mmol) 1-cyclopropytethanone, 112 mg (2.00 mmol) potassium hydroxide, 208 μL (3.63 mmol) acetic acid and 209 mg (2.54 mmol) sodium acetate in 3 mL water for 6 h at 150° C. in the microwave. The reaction mixture was quenched with 10% hydrochloric acid solution and filtered. The filtrate was extracted with ethyl acetate and the combined organic layers were washed with brine, dried over sodium sulfate, filtered and evaporated to obtain 120 mg (0.52 mmol, 29%) of the desired title compound after drying.

1H NMR (400 MHz, DMSO d₆): δ (ppm)=1.10-1.17 (m, 4H), 2.40-2.47 (m, 1H), 7.52-7.60 (m, 2H), 7.93 (s, 1H), 8.35-8.41 (m, 1H) 13.98 (br. s, 1H).

### Intermediate 26A

### 2-cyclopropyl-6-fluoroquinoline-4-carboxylic acid

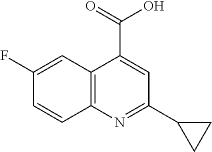 [SMILES: O=C(O)c1cc(C2CC2)nc2ccc(F)cc12]

In analogy to intermediate 25A, 300 mg (1.82 mmol) 5-fluoro-1H-indole-2,3-dione was heated with 900 μL (9.08 mmol) 1-cyclopropylethanone, 112 mg (2.00 mmol) potassium hydroxide, 208 μL (3.63 mmol) acetic acid and 209 mg (2.54 mmol) sodium acetate in 3 mL water for 24 h to reflux. The reaction mixture was filtered, the filtrate extracted with ethyl acetate and the combined organic layers were dried over sodium sulfate, filtered and evaporated to obtain 381 mg (1.65 mmol, 90%) of the desired title compound after drying.

1H NMR (400 MHz, DMSO d₆): δ (ppm)=0.54-0.64 (m, 1H), 0.66-0.83 (m, 3H), 1.92-2.04 (m, 1H), 6.09 (br. s., 1H), 6.74 (dd, 1H), 6.98 (ddd, 1H), 7.16 (dd, 1H), 10.19 (s, 1H).

### Intermediate 27A

### 8-bromo-2-cyclopropylquinoline-4-carboxylic acid

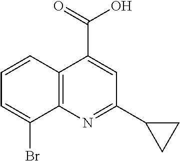 [SMILES: O=C(O)c1cc(C2CC2)nc2c(Br)cccc12]

In analogy to intermediate 25A, 300 mg (1.33 mmol) 7-bromo-1H-indole-2,3-dione was heated with 658 μL (6.64 mmol) 1-cyclopropylethanone, 81 mg (1.46 mmol) potassium hydroxide, 152 μL (2.65 mmol) acetic acid and 152 mg (1.86 mmol) sodium acetate in 3 mL water for 24 h at reflux. Because of low conversion the reaction mixture was heated for additional 6 h at 150° C. in the microwave. The reaction mixture was quenched with 10% hydrochloric acid solution and filtered. The filtrate was extracted with ethyl acetate and the combined organic layers were washed with brine, dried over sodium sulfate, filtered and evaporated. The crude reaction mixture was purified via preparative HPLC according to method 5d to obtain 100 mg (0.34 mmol, 26%) of the desired title compound after drying.

1H NMR (300 MHz, DMSO d₆): δ (ppm)=1.10-1.22 (m, 4H), 2.40-2.47 (m, 1H), 7.49 (dd, 1H), 7.94 (s, 1H), 8.13 (dd, 1H), 8.57 (dd, 1H), 14.02 (br. s, 1H).

### Intermediate 28A

### 2-cyclopropyl-8-fluoroquinoline-4-carboxylic acid

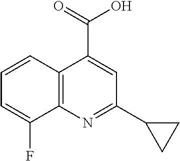 [SMILES: O=C(O)c1cc(C2CC2)nc2c(F)cccc12]

In analogy to intermediate 25A, 300 mg (1.82 mmol) 7-fluoro-1H-indole-2,3-dione was heated with 900 μL (9.08 mmol) 1-cyclopropylethanone, 112 mg (2.00 mmol) potassium hydroxide, 208 μL (3.63 mmol) acetic acid and 209 mg (2.54 mmol) sodium acetate in 3 mL water for 24 h at reflux. Because of low conversion the reaction mixture was heated for additional 6 h at 150° C. in the microwave. The reaction mixture was quenched with 10% hydrochloric acid solution and filtered. The filtrate was extracted with ethyl acetate and the combined organic layers were washed with brine, dried over sodium sulfate, filtered and evaporated. The crude reaction mixture was purified via preparative HPLC according to method 5c to obtain 94 mg (0.41 mmol, 22%) of the desired title compound after drying.

1H NMR (400 MHz, DMSO d₆): δ (ppm)=1.10-1.15 (m, 4H), 2.40-2.48 (m, 1H), 7.51-7.62 (m, 2H), 7.92 (s, 1H), 8.34-8.42 (m, 1H), 13.96 (br. s., 1H).

### Intermediate 29A

### 7-bromo-2-cyclopropylquinoline-4-carboxylic acid

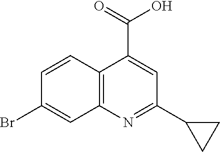 [SMILES: O=C(O)c1cc(C2CC2)nc2cc(Br)ccc12]

In analogy to intermediate 25A, 300 mg (1.33 mmol) 6-bromo-1H-indole-2,3-dione was heated with 658 μL (6.64 mmol) 1-cyclopropylethanone, 81 mg (1.50 mmol) potassium hydroxide, 152 μL (2.65 mmol) acetic acid and 152 mg (1.86 mmol) sodium acetate in 3 mL water for 4 h at 160° C. in the microwave. After aqueous work-up the crude reaction mixture was purified via preparative HPLC according to method 5c to obtain 80 mg (0.74 mmol, 21%) of the desired title compound after drying.

1H NMR (300 MHz, DMSO d₆): δ (ppm)=1.02-1.17 (m, 4H) 2.41 (quin, 1H) 7.73 (dd, 1H) 7.90 (s, 1H) 8.11 (d, 1H) 8.55 (d, 1H) 13.99 (br. s., 1H).

### Intermediate 30A

### 6-bromo-2-cyclobutylquinoline-4-carboxylic acid

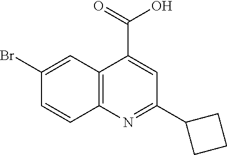 [SMILES: O=C(O)c1cc(C2CCC2)nc2ccc(Br)cc12]

In analogy to intermediate 1A) we obtained from 3.0 g (13.3 mmol) commercially available 5-bromo-1H-indole-2,3-dione and 2.61 g (26.5 mmol) of 1-cyclobutylethanone instead of 1,1,1-trifluoroacetone and using conventional heating at 100° C. for 16 hours reaction mixture which after cooling was filtered. The filtrate was extracted with ethyl acetate. The organic phase was dried over sodium sulfate, filtered and after the addition of Isolute® evaporated to dryness. The absorbed material was then purified using a Biotage chromatography system (50 g snap KP-Sil column, hexane/0-100% ethyl acetate, then ethyl acetate/0-100% methanol) getting impure material which was dissolved in 100 mL saturated aq. sodium hydrogencarbonate solution and stirred for 30 minutes at 25° C. Then this aq. mixture was extracted twice with 50 mL ethyl acetate. The aqueous phase was then acidified using 10% sulfuric acid up to pH 3. The formed solid was isolated by filtration and dried. Using this methodology we obtained the desired title compound. Yield: 90 mg (2.0%)

¹H-NMR (400 MHz, DMSO d₆) δ (ppm)=1.83-1.93 (m, 1H), 1.99-2.13 (m, 1H), 2.30-2.43 (m, 4H), 3.84-3.96 (m, 1H), 7.84 (s, 1H), 7.89 (dd, 1H), 7.98 (d, 1H), 8.91 (d, 1H), 13.42 (br. s., 1H).

### Intermediate 31A

### 8-bromo-2-methylquinoline-4-carboxylic acid

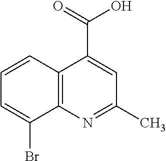

In analogy to intermediate 25A, 2 g (8.85 mmol) 7-bromo-1H-indole-2,3-dione was heated with 3.25 mL (44.24 mmol) acetone, 546 mg (9.73 mmol) potassium hydroxide, 1.01 mL (17.70 mmol) acetic acid and 1.02 g (12.39 mmol) sodium acetate in 20 mL water for 2 h at 130° C. in the microwave. As the conversion was not complete further 3.25 mL (44.24 mmol) acetone was added to the reaction mixture and heated again for 2 h at 130° C. in the microwave to obtain 2.00 g (7.51 mmol, 85%) of the desired title compound after aqueous work-up.

1H NMR (300 MHz, DMSO d₆): δ (ppm)=2.76 (s, 3H), 7.46-7.60 (m, 1H), 7.91 (s, 1H), 8.13-8.24 (m, 1H), 8.57-8.68 (m, 1H), 14.02 (br. s., 1H).

### Intermediate 32A

### 2-carbamoyl-6-chloro-7-fluoroquinoline-4-carboxylic acid

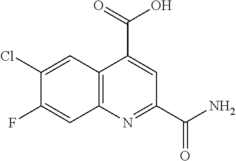 [SMILES: NC(=O)c1cc(C(=O)O)c2cc(Cl)c(F)cc2n1]

### Step 1: 6-chloro-7-fluoroquinoline-2,4-dicarboxylic acid

In analogy to step 1) of intermediate 2A) 10.0 g (50.1 mmol) commercially available 5-chloro-6-fluoro-1H-indole-2,3-dione were reacted to give 3.63 g (25%) 6-chloro-7-fluoroquinoline-2,4-dicarboxylic acid.

¹H-NMR (400 MHz, DMSO d₆) δ (ppm)=8.14 (d, 1H), 8.38 (s, 1H), 9.19 (d, 1H).

### Step 2: dimethyl 6-chloro-7-fluoroquinoline-2,4-dicarboxylate

In analogy to step 2) of intermediate 2A) 3.63 g (13.5 mmol) 6-chloro-7-fluoroquinoline-2,4-dicarboxylic acid of step 1) of intermediate 32A) were reacted to give 3.12 g (74%) dimethyl 6-chloro-7-fluoroquinoline-2,4-dicarboxylate.

¹H-NMR (500 MHz, DMSO d₆) δ (ppm)=3.98 (s, 3H), 4.01 (s, 3H), 8.29 (d, 1H), 8.46 (s, 1H), 8.94 (d, 1H).

### Step 3: methyl 2-carbamoyl-6-chloro-7-fluoroquinoline-4-carboxylate

In analogy to step 3) of intermediate 2A) 3.12 g (10.5 mmol) dimethyl 6-chloro-7-fluoroquinoline-2,4-dicarboxylate of step 2) of intermediate 32A) were reacted to give 2.52 g (77%) methyl 2-carbamoyl-6-chloro-7-fluoroquinoline-4-carboxylate.

¹H-NMR (400 MHz, DMSO d₆) δ (ppm)=4.01 (s, 3H), 7.98 (br. s., 1H), 8.11 (d, 1H), 8.36 (br. s., 1H), 8.54 (s, 1H), 8.96 (d, 1H).

### Step 4: 2-carbamoyl-6-chloro-7-fluoroquinoline-4-carboxylic acid

In analogy to step 4) of intermediate 2A) 520 mg (1.84 mmol) methyl 2-carbamoyl-6-chloro-7-fluoroquinoline-4-carboxylate of step 3) of intermediate 32A) were reacted to give 390 mg (63%) of the desired title compound.

¹H-NMR (400 MHz, DMSO d₆) δ (ppm)=7.92 (br. s., 1H), 8.06 (d, 1H), 8.31 (br. s., 1H), 8.51 (s, 1H), 9.10 (d, 1H).

### Intermediate 33A

### 5-bromo-2-(trifluoromethyl)quinoline-4-carboxylic acid

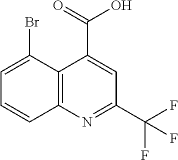 [SMILES: O=C(O)c1cc(C(F)(F)F)nc2cccc(Br)c12]

In analogy to intermediate 25A), 300 mg (1.33 mmol) 4-bromo-1H-indole-2,3-dione was heated with 238 μL (2.65 mmol) 1,1,1-trifluoroacetone, 82 mg (1.46 mmol) potassium hydroxide, 152 μL (2.65 mmol) acetic acid and 152 mg (1.86 mmol) sodium acetate in 5 mL water for 2 h at 120° C. in the microwave. The reaction mixture was quenched with 10% hydrochloric acid solution and filtered to get 309 mg (0.97 mmol, 73%) of the desired title compound.

1H NMR (300 MHz, DMSO d₆): δ (ppm)=7.90 (dd, 1H), 8.15 (s, 1H), 8.24 (dd, 1H), 8.30 (dd, 1H), 14.33 (br. s., 1H).

### Intermediate 34A

### 2-cyclopropyl-6-(trifluoromethyl)quinoline-4-carboxylic acid

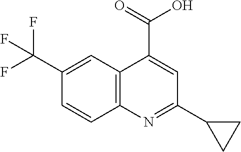 [SMILES: O=C(O)c1cc(C2CC2)nc2ccc(C(F)(F)F)cc12]

In analogy to intermediate 2A, 200 mg (0.93 mmol) 5-(trifluoromethyl)-1H-indole-2,3-dione was heated with 230 μL (2.32 mmol) 1-cyclopropylethanone, 156 mg (2.79 mmol) potassium hydroxide in 3 mL ethanol and 500 μL water for 6 h at 130° C. in the microwave. The reaction mixture was quenched with 10% hydrochloric acid solution, extracted with ethyl acetate and the combined organic layers were washed with brine, dried over sodium sulfate, filtered and evaporated. The crude reaction mixture was purified via preparative HPLC according to method 5d to obtain 18 mg (0.06 mmol, 7%) of the desired title compound after drying.

Method 1: Rₜ=1.15 min

MS (ESIpos): m/z=282 (M+H)⁺

### Intermediate 35A

### 8-fluoro-2-(trifluoromethyl)quinoline-4-carboxylic acid

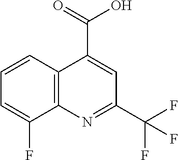 [SMILES: O=C(O)c1cc(C(F)(F)F)nc2c(F)cccc12]

In analogy to intermediate 25A, 100 mg (0.61 mmol) 7-fluoro-1H-indole-2,3-dione was heated with 543 μL (6.06 mmol) 1,1,1-trifluoroacetone, 37 mg (0.66 mmol) potassium hydroxide, 69 μL (1.21 mmol) acetic acid and 70 mg (0.85 mmol) sodium acetate in 2.5 mL water for 15 h at 70° C. The reaction mixture was quenched with 1 M hydrochloric acid solution and filtered. This crude product was purified via preparative HPLC according to method 5c to obtain 48 mg (0.19 mmol, 31%) of the desired title compound after drying.

1H NMR (300 MHz, DMSO d₆): δ (ppm)=7.82-7.95 (m, 2H), 8.35 (s, 1H), 8.60 (d, 1H), 14.52 (br. s., 1H).

### Intermediate 36A

### 2-cyclopropyl-8-(trifluoromethyl)quinoline-4-carboxylic acid

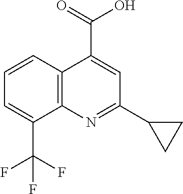 [SMILES: O=C(O)c1cc(C2CC2)nc2c(C(F)(F)F)cccc12]

In analogy to intermediate 25A, 300 mg (1.39 mmol) 7-(trifluoromethyl)-1H-indole-2,3-dione was heated with 238 μL (2.65 mmol) 1-cyclopropylethanone, 86 mg (1.53 mmol) potassium hydroxide, 160 μL (2.79 mmol) acetic acid and 160 mg (1.96 mmol) sodium acetate in 5 mL water for 4 h at 160° C. in the microwave. The reaction mixture was quenched with 1 M hydrochloric acid solution and filtered. This crude product was purified via preparative HPLC according to method 5d to obtain 85 mg (0.30 mmol, 22%) of the desired title compound after drying.

1H NMR (300 MHz, DMSO d₆): δ (ppm)=1.07-1.20 (m, 4H), 2.41-2.48 (m, 1H), 7.71 (t, 1H), 8.04 (s, 1H), 8.16 (d, 1H), 8.86 (d, 1H), 14.08 (br. s., 1H).

### Intermediate 37A

### 2-carbamoyl-7-fluoroquinoline-4-carboxylic acid

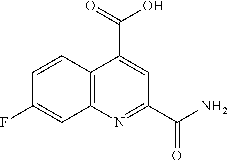 [SMILES: NC(=O)c1cc(C(=O)O)c2ccc(F)cc2n1]

### Step 1:

In analogy to step 1) of intermediate 2A) we got from 5.0 g (30.3 mmol) commercially available 6-fluoro-1H-indole-2,3-dione 6.02 g (84%) 7-fluoroquinoline-2,4-dicarboxylic acid.

¹H-NMR (300 MHz, DMSO d₆) δ (ppm)=7.78 (ddd, 1H), 7.99 (dd, 1H), 8.42 (s, 1H), 8.89 (dd, 1H).

### Step 2:

In analogy to step 2) of intermediate 2A) we got from 6.0 g (25.5 mmol) 7-fluoroquinoline-2,4-dicarboxylic acid of step 1) of intermediate 37A) 3.06 g (44%) dimethyl 7-fluoroquinoline-2,4-dicarboxylate.

¹H-NMR (300 MHz, DMSO d₆) δ (ppm)=3.98 (s, 3H), 4.01 (s, 3H), 7.85 (ddd, 1H), 8.07 (dd, 1H), 8.45 (s, 1H), 8.80 (dd, 1H).

### Step 3:

In analogy to step 3) of intermediate 2A) we got from 3.05 g (11.6 mmol) dimethyl 7-fluoroquinoline-2,4-dicarboxylate of step 2) of intermediate 37A) 2.33 g (81%) methyl 2-carbamoyl-7-fluoroquinoline-4-carboxylate.

¹H-NMR (400 MHz, DMSO d₆) δ (ppm)=4.03 (s, 3H), 7.83 (ddd, 1H), 7.94 (dd, 1H), 7.97 (s, 1H), 8.39 (s, 1H), 8.52 (s, 1H), 8.83 (dd, 1H).

### Step 4:

In analogy to step 4) of intermediate 2A) we got from 3.0 g (12.1 mmol) methyl 2-carbamoyl-7-fluoroquinoline-4-carboxylate of step 3) of intermediate 37A) 2.38 g (80%) of the desired title compound.

¹H-NMR (300 MHz, DMSO d₆) δ (ppm)=7.76 (ddd, 1H), 7.84-7.96 (m, 2H), 8.35 (br. s., 1H), 8.46 (s, 1H), 8.89 (dd, 1H), 14.02 (br. s., 1H).

### Intermediate 38A

### 2-carbamoyl-6-chloroquinoline-4-carboxylic acid

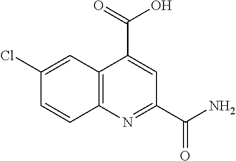 [SMILES: NC(=O)c1cc(C(=O)O)c2cc(Cl)ccc2n1]

### Step 1:

In analogy to step 1) of intermediate 2A) we got from 3.9 g (21.5 mmol) commercially available 5-chloro-1H-indole-2,3-dione 2.71 g (49%) 6-chloroquinoline-2,4-dicarboxylic acid.

¹H-NMR (300 MHz, DMSO d₆) δ (ppm)=7.96 (dd, 1H), 8.26 (d, 1H), 8.53 (s, 1H), 8.92 (d, 1H), 13.94 (br. s., 1H).

### Step 2:

In analogy to step 2) of intermediate 2A) we got from 2.7 g (10.7 mmol) 6-chloroquinoline-2,4-dicarboxylic acid of step 1) of intermediate 38A) 2.3 g (74%) dimethyl 6-chloroquinoline-2,4-dicarboxylate.

¹H-NMR (400 MHz, DMSO d₆) δ (ppm)=3.98 (s, 3H), 4.01 (s, 3H), 7.99 (dd, 1H), 8.29 (d, 1H), 8.51 (s, 1H), 8.79 (d, 1H).

### Step 3:

In analogy to step 3) of intermediate 2A) we got from 1.74 g (6.21 mmol) dimethyl 6-chloroquinoline-2,4-dicarboxylate of step 2) of intermediate 38A) 1.51 g (87%) methyl 2-carbamoyl-6-chloroquinoline-4-carboxylate.

¹H-NMR (400 MHz, DMSO d₆) δ (ppm)=4.02 (s, 3H), 7.96 (br. s., 1H), 8.00 (dd, 1H), 8.24 (d, 1H), 8.42 (br. s., 1H), 8.60 (s, 1H), 8.82 (d, 1H).

### Step 4:

In analogy to step 4) of intermediate 2A) we got from a first experiment with 0.5 g (1.9 mmol) and a second experiment with 1.0 g (3.78 mmol) methyl 2-carbamoyl-6-chloroquinoline-4-carboxylate of step 3) of intermediate 38A) a combined yield of 628 mg (60%) of the desired title compound.

¹H-NMR (400 MHz, DMSO d₆) δ (ppm)=7.90 (br. s., 1H), 7.95 (dd, 1H), 8.21 (d, 1H), 8.37 (br. s., 1H), 8.57 (s, 1H), 8.91 (d, 1H), 14.20 (br. s., 1H).

### Intermediate 39A

### 2-carbamoyl-5-fluoroquinoline-4-carboxylic acid

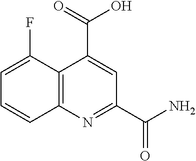 [SMILES: NC(=O)c1cc(C(=O)O)c2c(F)cccc2n1]

### Step 1:

In analogy to step 1) of intermediate 2A) we got from 5.07 g (30.7 mmol) commercially available 4-fluoro-1H-indole-2,3-dione 4.62 g (64%) 5-fluoroquinoline-2,4-dicarboxylic acid.

¹H-NMR (400 MHz, DMSO d₆) δ (ppm)=7.76 (ddd, 1H), 7.97 (dd, 1H), 8.38 (s, 1H), 8.90 (dd, 1H).

### Step 2:

In analogy to step 2) of intermediate 2A) we got from 4.62 g (19.6 mmol) 5-fluoroquinoline-2,4-dicarboxylic acid of step 1) of intermediate 39A) 4.72 g (80%) dimethyl 5-fluoroquinoline-2,4-dicarboxylate.

¹H-NMR (300 MHz, DMSO d₆) δ (ppm)=3.98 (s, 3H), 4.01 (s, 3H), 7.84 (ddd, 1H), 8.06 (dd, 1H), 8.44 (s, 1H), 8.80 (dd, 1H).

### Step 3:

In analogy to step 3) of intermediate 2A) we got from 4.70 g (17.9 mmol) dimethyl 5-fluoroquinoline-2,4-dicarboxylate of step 2) of intermediate 39A) 3.29 g (72%) methyl 2-carbamoyl-5-fluoroquinoline-4-carboxylate.

¹H-NMR (400 MHz, DMSO d₆) δ (ppm)=4.01 (s, 3H), 7.80 (ddd, 1H), 7.89-7.96 (m, 2H), 8.36 (br. s., 1H), 8.50 (s, 1H), 8.80 (dd, 1H).

### Step 4:

In analogy to step 4) of intermediate 2A) we got from 1.5 g (6.04 mmol) methyl 2-carbamoyl-5-fluoroquinoline-4-carboxylate of step 3) of intermediate 39A) 1.02 g (72%) of the desired title compound.

¹H-NMR (400 MHz, DMSO d₆) δ (ppm)=7.73-7.82 (m, 1H), 7.86-7.94 (m, 2H), 8.35 (s, 1H), 8.47 (s, 1H), 8.90 (dd, 1H), 13.97 (br. s., 1H).

### Intermediate 40A

### 2-carbamoyl-5-methylquinoline-4-carboxylic acid

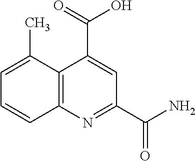 [SMILES: Cc1cccc2nc(C(N)=O)cc(C(=O)O)c12]

### Step 1:

In analogy to step 1) of intermediate 2A) we got from 2.0 g (12.4 mmol) commercially available 4-methyl-1H-indole-2,3-dione 1.64 g (49%) 5-methylquinoline-2,4-dicarboxylic acid.

¹H-NMR (400 MHz, DMSO d₆) δ (ppm)=2.67-2.75 (m, 3H), 7.62-7.70 (m, 1H), 7.82 (dd, 1H), 8.00-8.05 (m, 1H), 8.09 (d, 1H).

### Step 2:

In analogy to step 2) of intermediate 2A) we got from 1.64 g (7.09 mmol) 5-methylquinoline-2,4-dicarboxylic acid of step 1) of intermediate 40A) 2.1 g (110%, raw material) dimethyl 5-methylquinoline-2,4-dicarboxylate.

¹H-NMR (400 MHz, DMSO d₆) δ (ppm)=2.57 (s, 3H), 4.01 (s, 3H), 3.98 (s, 3H), 7.70 (d, 1H), 7.82-7.89 (m, 1H), 8.12 (d, 1H), 8.16 (s, 1H).

### Step 3:

In analogy to step 3) of intermediate 2A) we got from 2.1 g (8.1 mmol) dimethyl 5-methylquinoline-2,4-dicarboxylate of step 2) of intermediate 40A) 1.90 g (91%) methyl 2-carbamoyl-5-methylquinoline-4-carboxylate.

¹H-NMR (400 MHz, DMSO d₆) δ (ppm)=2.55 (s, 3H), 3.99 (s, 3H), 7.64 (dt, 1H), 7.82 (dd, 1H), 7.86 (br. s., 1H), 8.06 (d, 1H), 8.12 (s, 1H), 8.33 (br. s., 1H).

### Step 4:

In analogy to step 4) of intermediate 2A) we got from 1.9 g (12.1 mmol) methyl 2-carbamoyl-5-methylquinoline-4-carboxylate of step 3) of intermediate 40A) no solid, therefore the aqueous solution was extracted 3 times with ethyl acetate. The combined organic phases were dried over sodium sulfate, filtered and then the filtrate was evaporated in vacuum to dryness. This solid was stirred in a mixture of ethyl acetate and methanol to give a remaining solid, which was dried to yield 180 mg (9.4%) of the desired title compound.

¹H-NMR (400 MHz, DMSO d₆) δ (ppm)=2.67 (s, 3H), 7.58 (d, 1H), 7.78 (dd, 1H), 7.82 (br. s., 1H), 7.98 (s, 1H), 8.03 (d, 1H), 8.30 (br. s., 1H).

### Intermediate 41A

### 2-carbamoyl-6-methylquinoline-4-carboxylic acid

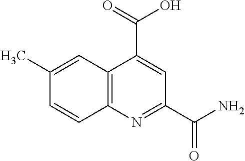 [SMILES: Cc1ccc2nc(C(N)=O)cc(C(=O)O)c2c1]

### Step 1:

In analogy to step 1) of intermediate 2A) we got from 5.13 g (31.8 mmol) commercially available 5-methyl-1H-indole-2,3-dione 4.22 g (56%) 6-methylquinoline-2,4-dicarboxylic acid.

¹H-NMR (400 MHz, DMSO d₆) δ (ppm)=2.59 (s, 3H), 2.63-2.75 (m, 1H), 7.79 (dd, 1H), 8.14 (d, 1H), 8.44 (s, 1H), 8.57 (s, 1H).

### Step 2:

In analogy to step 2) of intermediate 2A) we got from 4.2 g (18.2 mmol) 6-methylquinoline-2,4-dicarboxylic acid of step 1) of intermediate 41A) 3.17 g (54%) dimethyl 6-methylquinoline-2,4-dicarboxylate.

¹H-NMR (300 MHz, DMSO d₆) δ (ppm)=2.58 (s, 3H), 4.00 (s, 3H), 3.96 (s, 3H), 7.80 (dd, 1H), 8.15 (d, 1H), 8.41-8.51 (m, 2H).

### Step 3:

In analogy to step 3) of intermediate 2A) we got from 3.17 g (12.2 mmol) dimethyl 6-methylquinoline-2,4-dicarboxylate of step 2) of intermediate 41A) 2.72 g (88%) methyl 2-carbamoyl-6-methylquinoline-4-carboxylate.

¹H-NMR (300 MHz, DMSO d₆) δ (ppm)=2.57 (s, 3H), 4.00 (s, 3H), 7.78 (dd, 1H), 7.86 (br. s., 1H), 8.10 (d, 1H), 8.34 (br. s., 1H), 8.40-8.53 (m, 2H).

### Step 4:

In analogy to step 4) of intermediate 2A) we got from 500 mg (2.05 mmol) methyl 2-carbamoyl-6-methylquinoline-4-carboxylate of step 3) of intermediate 41A) 480 mg (102%, some moisture included) of the desired title compound.

¹H-NMR (300 MHz, DMSO d₆) δ (ppm)=2.56 (s, 3H), 7.76 (dd, 1H), 7.84 (br. s., 1H), 8.09 (d, 1H), 8.32 (br. s., 1H), 8.46 (s, 1H), 8.55 (s, 1H), 13.95 (br. s., 1H).

### Intermediate 42A

### 2-carbamoyl-7-methoxyquinoline-4-carboxylic acid

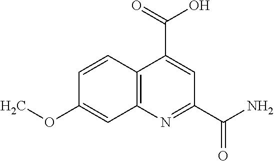 [SMILES: COc1ccc2c(C(=O)O)cc(C(N)=O)nc2c1]

### Step 1:

In analogy to step 1) of intermediate 2A) we got from 5.0 g (28.2 mmol) commercially available 6-methoxy-1H-indole-2,3-dione 2.71 g (38%) 7-methoxyquinoline-2,4-dicarboxylic acid.

¹H-NMR (300 MHz, DMSO d₆) δ (ppm)=3.96 (s, 3H), 7.49 (dd, 1H), 7.60 (d, 1H), 8.31 (s, 1H), 8.69 (d, 1H).

### Step 2:

In analogy to step 2) of intermediate 2A) we got from 2.7 g (10.9 mmol) 7-methoxyquinoline-2,4-dicarboxylic acid of step 1) of intermediate 42A) 549 mg (32%) dimethyl 7-methoxyquinoline-2,4-dicarboxylate.

¹H-NMR (400 MHz, DMSO d₆) δ (ppm)=3.98 (s, 3H), 3.99 (s, 3H), 4.01 (s, 3H), 7.54 (dd, 1H), 7.67 (d, 1H), 8.34 (s, 1H), 8.63 (d, 1H).

### Step 3:

In analogy to step 3) of intermediate 2A) we got from 545 mg (1.98 mmol) dimethyl 7-methoxyquinoline-2,4-dicarboxylate of step 2) of intermediate 42A) 408 mg (79%) methyl 2-carbamoyl-7-methoxyquinoline-4-carboxylate.

¹H-NMR (400 MHz, DMSO d₆) δ (ppm)=3.98 (s, 3H), 4.01 (s, 3H), 7.51 (dd, 1H), 7.58 (d, 1H), 7.88 (br. s., 1H), 8.34 (br. s., 1H), 8.39 (s, 1H), 8.63 (d, 1H).

### Step 4:

In analogy to step 4) of intermediate 2A) we got from 400 mg (1.54 mmol) methyl 2-carbamoyl-7-methoxyquinoline-4-carboxylate of step 3) of intermediate 42A) 349 mg (44%) of the desired title compound.

¹H-NMR (300 MHz, DMSO d₆) δ (ppm)=3.95 (s, 3H), 7.38-7.46 (m, 1H), 7.53 (dd, 1H), 7.79 (br. s., 1H), 8.18-8.30 (m, 2H), 8.67 (dd, 1H).

### Intermediate 43A

### 2-carbamoyl-8-chloroquinoline-4-carboxylic acid

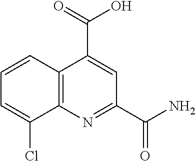 [SMILES: NC(=O)c1cc(C(=O)O)c2cccc(Cl)c2n1]

### Step 1:

In analogy to step 1) of intermediate 2A) we got from 5.0 g (27.5 mmol) commercially available 7-chloro-1H-indole-2,3-dione 3.34 g (48%) 8-chloroquinoline-2,4-dicarboxylic acid.

¹H-NMR (300 MHz, DMSO d₆) δ (ppm)=6.57 (t, 1H), 7.15 (br. s., 2H), 7.47 (ddd, 2H), 7.69-7.79 (m, 1H), 8.05-8.13 (m, 1H), 8.41 (s, 1H), 8.72 (d, 1H).

### Step 2:

In analogy to step 2) of intermediate 2A) we got from 3.34 g (13.3 mmol) 8-chloroquinoline-2,4-dicarboxylic acid of step 1) of intermediate 43A) 2.22 g (58%) dimethyl 8-chloroquinoline-2,4-dicarboxylate.

¹H-NMR (300 MHz, DMSO d₆) δ (ppm)=3.99 (s, 3H), 4.01 (s, 3H), 7.84 (dd, 1H), 8.16 (dd, 1H), 8.53 (s, 1H), 8.66 (dd, 1H).

### Step 3:

In analogy to step 3) of intermediate 2A) we got from 2.24 g (8.01 mmol) dimethyl 8-chloroquinoline-2,4-dicarboxylate of step 2) of intermediate 43A) 1.69 g (76%) methyl 2-carbamoyl-8-chloroquinoline-4-carboxylate.

¹H-NMR (400 MHz, DMSO d₆) δ (ppm)=4.02 (s, 3H), 7.81 (dd, 1H), 8.04-8.10 (m, 2H), 8.14 (dd, 1H), 8.58 (s, 1H), 8.66 (dd, 1H).

### Step 4:

In analogy to step 4) of intermediate 2A) we got from 1.68 g (6.35 mmol) methyl 2-carbamoyl-8-chloroquinoline-4-carboxylate of step 3) of intermediate 43A) 1.60 g (100%) of the desired title compound.

¹H-NMR (300 MHz, DMSO d₆) δ (ppm)=7.81 (ddd, 1H), 8.04-8.10 (m, 1H), 8.14 (d, 1H), 8.51 (s, 1H), 8.57 (s, 1H), 8.76 (d, 1H), 14.16 (s, 1H).

### Intermediate 44A

### 2-carbamoyl-5,7-difluoroquinoline-4-carboxylic acid

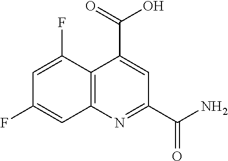 [SMILES: NC(=O)c1cc(C(=O)O)c2c(F)cc(F)cc2n1]

### Step 1:

In analogy to step 1) of intermediate 2A) we got from 1.85 g (10.1 mmol) commercially available 4,6-difluoro-1H-indole-2,3-dione 850 mg (32%) 5,7-difluoroquinoline-2,4-dicarboxylic acid.

¹H-NMR (400 MHz, DMSO d₆) δ (ppm)=7.74 (ddd, 1H), 7.80-7.87 (m, 1H), 7.91 (s, 1H).

### Step 2:

In analogy to step 2) of intermediate 2A) we got from 850 mg (3.36 mmol) 5,7-difluoroquinoline-2,4-dicarboxylic acid of step 1) of intermediate 44A) 910 mg (92%) dimethyl 5,7-difluoroquinoline-2,4-dicarboxylate.

¹H-NMR (300 MHz, DMSO d₆) δ (ppm)=3.96 (s, 3H), 3.97 (s, 3H), 7.91 (ddd, 1H), 8.00 (ddd, 1H), 8.24 (s, 1H).

### Step 3:

In analogy to step 3) of intermediate 2A) we got from 910 mg (3.24 mmol) dimethyl 5,7-difluoroquinoline-2,4-dicarboxylate of step 2) of intermediate 44A) 850 mg (92%) methyl 2-carbamoyl-5,7-difluoroquinoline-4-carboxylate.

¹H-NMR (300 MHz, DMSO d₆) δ (ppm)=3.96 (s, 3H), 7.79-7.94 (m, 2H), 8.00 (br. s., 1H), 8.21 (s, 1H), 8.38 (br. s., 1H).

### Step 4:

In analogy to step 4) of intermediate 2A) we got from 230 mg (0.86 mmol) methyl 2-carbamoyl-5,7-difluoroquinoline-4-carboxylate of step 3) of intermediate 44A) 150 mg (65%) of the desired title compound.

¹H-NMR (400 MHz, DMSO d₆) δ (ppm)=7.77-7.85 (m, 2H), 7.96 (br. s., 1H), 8.07 (s, 1H), 8.34 (br. s., 1H), 14.02 (br. s., 1H).

### Intermediate 45A

### 2-carbamoyl-7-fluoro-6-methoxyquinoline-4-carboxylic acid

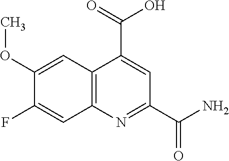 [SMILES: COc1cc2c(C(=O)O)cc(C(N)=O)nc2cc1F]

### Step 1:

In analogy to step 1) of intermediate 2A) we got from 3.11 g (15.9 mmol) commercially available 6-fluoro-5-methoxy-1H-indole-2,3-dione 1.3 g (30%) 7-fluoro-6-methoxyquinoline-2,4-dicarboxylic acid.

¹H-NMR (400 MHz, DMSO d₆) δ (ppm)=4.02 (s, 3H), 8.02 (d, 1H), 8.43 (d, 1H), 8.48 (s, 1H), 13.72 (br. s., 1H).

### Step 2:

In analogy to step 2) of intermediate 2A) we got from 1.3 g (4.9 mmol) 7-fluoro-6-methoxyquinoline-2,4-dicarboxylic acid of step 1) of intermediate 45A) 960 mg (57%) dimethyl 7-fluoro-6-methoxyquinoline-2,4-dicarboxylate.

¹H-NMR (400 MHz, DMSO d₆) δ (ppm)=3.97 (s, 3H), 4.03 (s, 3H), 4.05 (s, 3H), 8.10 (d, 1H), 8.33 (d, 1H), 8.48 (s, 1H).

### Step 3:

In analogy to step 3) of intermediate 2A) we got from 960 mg (3.27 mmol) dimethyl 7-fluoro-6-methoxyquinoline-2,4-dicarboxylate of step 2) of intermediate 45A) 520 mg (57%) crude methyl 2-carbamoyl-7-fluoro-6-methoxyquinoline-4-carboxylate which was used without further purification.

¹H-NMR (400 MHz, DMSO d₆) δ (ppm)=4.03 (s, 3H), 4.05 (s, 3H), 7.88 (br. s., 1H), 7.95 (d, 1H), 8.29 (br. s., 1H), 8.35 (d, 1H), 8.54 (s, 1H).

### Step 4:

In analogy to step 4) of intermediate 2A) we got from 520 mg (1.87 mmol) methyl 2-carbamoyl-7-fluoro-6-methoxyquinoline-4-carboxylate of step 3) of intermediate 45A) 440 mg (61%) of the desired title compound including about 31% of the corresponding diacid.

¹H-NMR (400 MHz, DMSO d₆) δ (ppm)=4.01 (s, 3H), 7.78 (br. s., 1H), 7.88 (d, 1H), 8.21 (br. s., 1H), 8.44-8.49 (m, 2H).

### Intermediate 46A

### 2-carbamoyl-6-methoxyquinoline-4-carboxylic acid

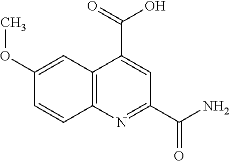 [SMILES: COc1ccc2nc(C(N)=O)cc(C(=O)O)c2c1]

### Step 1:

In analogy to step 1) of intermediate 2A) we got from 5.0 g (38.2 mmol) commercially available 5-methoxy-1H-indole-2,3-dione 3.01 g (42%) 6-methoxyquinoline-2,4-dicarboxylic acid.

¹H-NMR (400 MHz, DMSO d₆) δ (ppm)=3.94 (s, 3H), 7.57 (dd, 1H), 8.14 (d, 1H), 8.25 (d, 1H), 8.48 (s, 1H), 13.66 (br. s., 1H).

### Step 2:

In analogy to step 2) of intermediate 2A) we got from 3.0 g (12.1 mmol) 6-methoxyquinoline-2,4-dicarboxylic acid of step 1) of intermediate 46A) 2.65 g (77%) dimethyl 6-methoxyquinoline-2,4-dicarboxylate.

¹H-NMR (300 MHz, DMSO d₆) δ (ppm)=3.95 (s, 6H), 4.00 (s, 3H), 7.60 (dd, 1H), 8.13-8.20 (m, 2H), 8.48 (s, 1H).

### Step 3:

In analogy to step 3) of intermediate 2A) we got from 2.65 g (9.63 mmol) dimethyl 6-methoxyquinoline-2,4-dicarboxylate of step 2) of intermediate 46A) 1.45 g (55%) methyl 2-carbamoyl-6-methoxyquinoline-4-carboxylate.

¹H-NMR (400 MHz, DMSO d₆) δ (ppm)=3.97 (s, 3H), 4.03 (s, 3H), 7.62 (dd, 1H), 7.82 (br. s., 1H), 8.14 (d, 1H), 8.18 (d, 1H), 8.30 (br. s., 1H), 8.56 (s, 1H).

### Step 4:

In analogy to step 4) of intermediate 2A) we got from 1.45 g (5.57 mmol) methyl 2-carbamoyl-6-methoxyquinoline-4-carboxylate of step 3) of intermediate 46A) 1.33 g (92%) of the desired title compound.

¹H-NMR (400 MHz, DMSO d₆) δ (ppm)=3.93 (s, 3H), 7.57 (dd, 1H), 7.79 (s, 1H), 8.09 (d, 1H), 8.25 (d, 1H), 8.27 (br. s., 1H), 8.51 (s, 1H), 13.72 (br. s., 1H), 13.79-13.79 (m, 1H).

### Intermediate 47A

### 2-carbamoyl-8-fluoroquinoline-4-carboxylic acid

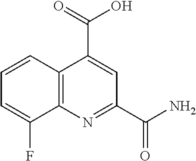 [SMILES: NC(=O)c1cc(C(=O)O)c2cccc(F)c2n1]

### Step 1:

In analogy to step 1) of intermediate 2A) we got from 5.36 g (32.5 mmol) commercially available 7-fluoro-1H-indole-2,3-dione 4.75 g (60%) 8-fluoroquinoline-2,4-dicarboxylic acid.

¹H-NMR (300 MHz, DMSO d₆) δ (ppm)=7.72-7.87 (m, 2H), 8.51 (s, 1H), 8.59 (d, 1H).

### Step 2:

In analogy to step 2) of intermediate 2A) we got from 4.70 g (20.0 mmol) 8-fluoroquinoline-2,4-dicarboxylic acid of step 1) of intermediate 47A) 4.40 g (72%) dimethyl 8-fluoroquinoline-2,4-dicarboxylate.

¹H-NMR (300 MHz, DMSO d₆) δ (ppm)=3.99 (s, 3H), 4.02 (s, 3H), 7.77-7.92 (m, 2H), 8.48-8.57 (m, 2H).

### Step 3:

In analogy to step 3) of intermediate 2A) we got from 4.40 g (16.7 mmol) dimethyl 8-fluoroquinoline-2,4-dicarboxylate of step 2) of intermediate 47A) 3.90 g (85%) methyl 2-carbamoyl-8-fluoroquinoline-4-carboxylate.

¹H-NMR (400 MHz, DMSO d₆) δ (ppm)=4.03 (s, 3H), 7.74-7.92 (m, 2H), 8.00 (br. s., 1H), 8.24 (s, 1H), 8.53 (d, 1H), 8.61 (s, 1H).

### Step 4:

In analogy to step 4) of intermediate 2A) we got from 500 mg (2.01 mmol) methyl 2-carbamoyl-8-fluoroquinoline-4-carboxylate of step 3) of intermediate 47A) 190 mg (40%) of the desired title compound.

¹H-NMR (300 MHz, DMSO d₆) δ (ppm)=7.71-7.85 (m, 2H), 7.96 (br. s., 1H), 8.20 (s, 1H), 8.53-8.62 (m, 2H).

### Intermediate 48A

### 2-carbamoyl-7-chloroquinoline-4-carboxylic acid

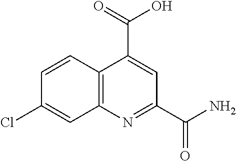 [SMILES: NC(=O)c1cc(C(=O)O)c2ccc(Cl)cc2n1]

### Step 1:

In analogy to step 1) of intermediate 2A) we got from 4.0 g (22.0 mmol) commercially available 6-chloro-1H-indole-2,3-dione 6.61 g (119%) crude 7-chloroquinoline-2,4-dicarboxylic acid which was used without further purification.

¹H-NMR (400 MHz, DMSO d₆) δ (ppm)=7.88 (dd, 1H), 8.29 (d, 1H), 8.47 (s, 1H), 8.84 (d, 1H).

### Step 2:

In analogy to step 2) of intermediate 2A) we got from 7.5 g (29.8 mmol) 7-chloroquinoline-2,4-dicarboxylic acid of step 1) of intermediate 48A) 4.60 g (55%) dimethyl 7-chloroquinoline-2,4-dicarboxylate.

¹H-NMR (300 MHz, DMSO d₆) δ (ppm)=3.98 (s, 3H), 4.01 (s, 3H), 7.92 (dd, 1H), 8.35 (d, 1H), 8.47 (s, 1H), 8.75 (d, 1H).

### Step 3:

In analogy to step 3) of intermediate 2A) we got from 4.50 g (16.1 mmol) dimethyl 7-fluoroquinoline-2,4-dicarboxylate of step 2) of intermediate 48A) 3.72 g (83%) methyl 2-carbamoyl-7-chloroquinoline-4-carboxylate.

¹H-NMR (300 MHz, DMSO d₆) δ (ppm)=4.00 (s, 3H), 7.89 (dd, 1H), 7.97 (br. s., 1H), 8.23 (d, 1H), 8.38 (br. s., 1H), 8.53 (s, 1H), 8.75 (d, 1H).

### Step 4:

In analogy to step 4) of intermediate 2A) we got from 400 mg (1.51 mmol) methyl 2-carbamoyl-7-chloroquinoline-4-carboxylate of step 3) of intermediate 48A) 363 mg (86%) of the desired title compound.

¹H-NMR (300 MHz, DMSO d₆) δ (ppm)=7.65 (dd, 1H), 7.75 (br. s., 1H), 8.06 (d, 1H), 8.18 (s, 1H), 8.22 (br. s., 1H), 8.86 (d, 1H).

### Intermediate 49A

### 2-carbamoyl-6-chloro-8-fluoroquinoline-4-carboxylic acid

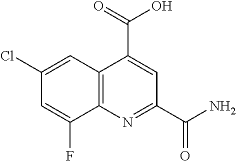 [SMILES: NC(=O)c1cc(C(=O)O)c2cc(Cl)cc(F)c2n1]

### Step 1:

In analogy to step 1) of intermediate 2A) we got from 2.5 g (12.5 mmol) commercially available 5-chloro-7-fluoro-1H-indole-2,3-dione 590 mg (17%) 6-chloro-8-fluoroquinoline-2,4-dicarboxylic acid.

¹H-NMR (400 MHz, DMSO d₆) δ (ppm)=7.99 (dd, 1H), 8.56 (s, 1H), 8.74-8.77 (m, 1H).

### Step 2:

In analogy to step 2) of intermediate 2A) we got from 590 mg (2.19 mmol) 6-chloro-8-fluoroquinoline-2,4-dicarboxylic acid of step 1) of intermediate 49A) 550 mg (76%) dimethyl 6-chloro-8-fluoroquinoline-2,4-dicarboxylate.

¹H-NMR (400 MHz, DMSO d₆) δ (ppm)=3.99 (s, 3H), 4.02 (s, 3H), 8.06 (dd, 1H), 8.57-8.62 (m, 2H).

### Step 3:

In analogy to step 3) of intermediate 2A) we got from 550 mg (1.85 mmol) dimethyl 6-chloro-8-fluoroquinoline-2,4-dicarboxylate of step 2) of intermediate 49A) 480 mg (83%) methyl 2-carbamoyl-6-chloro-8-fluoroquinoline-4-carboxylate.

¹H-NMR (400 MHz, DMSO d₆) δ (ppm)=4.01 (s, 3H), 7.98-8.07 (m, 2H), 8.23 (br. s., 1H), 8.60-8.62 (m, 1H), 8.63 (s, 1H).

### Step 4:

In analogy to step 4) of intermediate 2A) we got from 480 mg (1.70 mmol) methyl 2-carbamoyl-6-chloro-8-fluoroquinoline-4-carboxylate of step 3) of intermediate 49A) 430 mg (85%) of the desired title compound.

¹H-NMR (400 MHz, DMSO d₆) δ (ppm)=7.93-8.01 (m, 2H), 8.19 (br. s., 1H), 8.60 (s, 1H), 8.73-8.76 (m, 1H).

### Intermediate 50A

### 2-carbamoyl-7-fluoro-6-methylquinoline-4-carboxylic acid

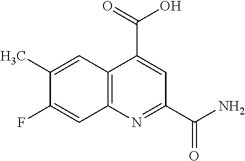 [SMILES: Cc1cc2c(C(=O)O)cc(C(N)=O)nc2cc1F]

### Step 1:

In analogy to step 1) of intermediate 2A) we got from 18.0 g (100 mmol) commercially available 6-fluoro-5-methyl-1H-indole-2,3-dione ([CAS-No. 749240-55-9] e.g. Fluorochem) 8.55 g (33%) 7-fluoro-6-methylquinoline-2,4-dicarboxylic acid.

¹H-NMR (400 MHz, DMSO d₆) δ (ppm)=2.51 (s, 3H), 7.95 (d, 1H), 8.41 (s, 1H), 8.72 (d, 1H).

### Step 2:

In analogy to step 2) of intermediate 2A) we got from 8.55 g (34.3 mmol) 7-fluoro-6-methylquinoline-2,4-dicarboxylic acid of step 1) of intermediate 50A) 6.9 g (69%) dimethyl 7-fluoro-6-methylquinoline-2,4-dicarboxylate.

¹H-NMR (400 MHz, DMSO d₆) δ (ppm)=2.51 (s, 3H), 3.98 (s, 3H), 4.02 (s, 3H), 7.99 (d, 1H), 8.40 (s, 1H), 8.61 (d, 1H).

### Step 3:

In analogy to step 3) of intermediate 2A) we got from 6.93 g (25.0 mmol) dimethyl 7-fluoro-6-methylquinoline-2,4-dicarboxylate of step 2) of intermediate 50A) 4.86 g (72%) methyl 2-carbamoyl-7-fluoro-6-methylquinoline-4-carboxylate.

¹H-NMR (400 MHz, DMSO d₆) δ (ppm)=2.50 (s, 3H), 4.00 (s, 3H), 7.85 (d, 1H), 7.89 (br. s., 1H), 8.31 (br. s., 1H), 8.45 (s, 1H), 8.60 (d, 1H).

### Step 4:

In analogy to step 4) of intermediate 2A) we got from 1.50 g (5.72 mmol) methyl 2-carbamoyl-7-fluoro-6-methylquinoline-4-carboxylate of step 3) of intermediate 50A) 1.34 g (90%) of the desired title compound.

¹H-NMR (400 MHz, DMSO d₆) δ (ppm)=2.51 (s, 3H), 7.85 (d, 1H), 7.89 (br. s., 1H), 8.32 (br. s., 1H), 8.45 (s, 1H), 8.71 (d, 1H), 13.94 (br. s., 1H).

### Intermediate 51A

### 6-fluoro-2-methoxyquinoline-4-carboxylic acid

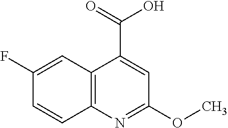 [SMILES: COc1cc(C(=O)O)c2cc(F)ccc2n1]

Starting from commercially available 6-fluoro-2-hydroxyquinoline-4-carboxylic acid ([607-40-9], e.g. ABCR) the title compound was prepared according to the described procedure in U.S. Pat. No. 6,699,879 of 2004.

¹H-NMR (400 MHz, DMSO d₆) δ (ppm)=4.01 (s, 3H), 7.49 (s, 1H), 7.64 (td, 1H), 7.91 (dd, 1H), 8.34 (dd, 1H), 14.00 (br. s., 1H).

### Intermediate 52A

### 2-(azetidin-1-ylcarbonyl)-7-fluoroquinoline-4-carboxylic acid

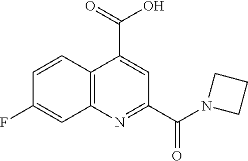 [SMILES: O=C(O)c1cc(C(=O)N2CCC2)nc2cc(F)ccc12]

### Step 1:

To a solution of 500 mg (1.90 mmol) dimethyl 7-fluoroquinoline-2,4-dicarboxylate of step 2) intermediate 37A) in 10 mL methanol was added 0.26 mL (3.80 mmol) azetidine. The reaction mixture was stirred for 14 hours at 24° C. The formed solid was isolated by filtration and dried. Using this methodology we obtained 390 mg (69%) of the desired methyl 2-(azetidin-1-ylcarbonyl)-7-fluoroquinoline-4-carboxylate.

¹H-NMR (400 MHz, DMSO d₆) δ (ppm)=2.30-2.40 (m, 2H), 4.02 (s, 3H), 4.16 (dd, 2H), 4.77 (t, 2H), 7.81 (ddd, 1H), 7.97 (dd, 1H), 8.40 (s, 1H), 8.79 (dd, 1H).

### Step 2:

In analogy to step 4) of intermediate 2A) we got from 390 mg (1.35 mmol) methyl 2-(azetidin-1-ylcarbonyl)-7-fluoroquinoline-4-carboxylate of step 1) of intermediate 52A) 210 mg (42%) of the desired title compound.

¹H-NMR (400 MHz, DMSO d₆) δ (ppm)=2.34 (quin, 2H), 4.16 (t, 2H), 4.76 (t, 2H), 7.76 (ddd, 1H), 7.93 (dd, 1H), 8.35 (s, 1H), 8.87 (dd, 1H), 14.07 (br. s., 1H).

### Intermediate 53A

### 7-fluoro-2-[(3-hydroxypropyl)carbamoyl]quinoline-4-carboxylic acid

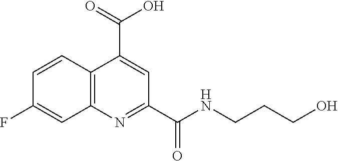 [SMILES: O=C(NCCCO)c1cc(C(=O)O)c2ccc(F)cc2n1]

### Step 1:

To a solution of 500 mg (1.90 mmol) dimethyl 7-fluoroquinoline-2,4-dicarboxylate of step 2) intermediate 37A) in 10 mL methanol was added 0.29 mL (3.80 mmol) 3-aminopropan-1-ol. The reaction mixture was stirred for 14 hours at 24° C. and then evaporated to dryness. The yielded mixture (850 mg) of the desired material methyl 7-fluoro-2-[(3-hydroxypropyl)carbamoyl]quinoline-4-carboxylate and the corresponding bisamide was used for the next step without any further purification.

### Step 2:

In analogy to step 4) of intermediate 2A) we got from 850 mg of a mixture of methyl 7-fluoro-2-[(3-hydroxypropyl)carbamoyl]quinoline-4-carboxylate and the corresponding bisamide of step 1) of intermediate 53A) 190 mg of crude material in which the desired title compound is included.

### Intermediate 54A

### 7-fluoro-2-{[2-(morpholin-4-yl)ethyl]carbamoyl}quinoline-4-carboxylic acid

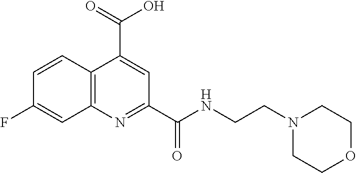 [SMILES: O=C(NCCN1CCOCC1)c1cc(C(=O)O)c2ccc(F)cc2n1]

### Step 1:

To a solution of 500 mg (1.90 mmol) dimethyl 7-fluoroquinoline-2,4-dicarboxylate of step 2) intermediate 37A) in 10 mL methanol was added 0.50 mL (3.80 mmol) 2-(morpholin-4-yl)ethanamine. The reaction mixture was stirred for 3 days at 24° C. and then evaporated to dryness. The yielded mixture (800 mg) of the desired material methyl 7-fluoro-2-{[2-(morpholin-4-yl)ethyl]carbamoyl}quinoline-4-carboxylate and the corresponding bisamide was used for the next step without any further purification.

### Step 2:

In analogy to step 4) of intermediate 2A) we got from 800 mg of a mixture of methyl 7-fluoro-2-{[2-(morpholin-4-yl)ethyl]carbamoyl}quinoline-4-carboxylate and the corresponding bisamide of step 1) of intermediate 54A) 250 mg of the a raw material in which the desired title compound is included.

### Intermediate 55A

### 2-carbamoyl-6-[(2-methoxyethyl)amino]quinoline-4-carboxylic acid

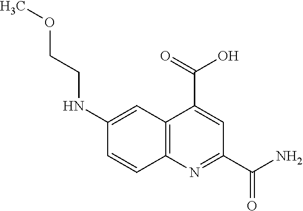 [SMILES: COCCNc1ccc2nc(C(N)=O)cc(C(=O)O)c2c1]

### Step 1:

To a solution of 3.00 g (9.71 mmol) methyl 6-bromo-2-carbamoylquinoline-4-carboxylate of step 3) intermediate 2A) in 60 mL toluene was added 2.49 g (10.2 mmol) bis(4-methoxyphenyl)methanol and 185 mg (0.97 mmol) p-toluenesulfonic acid and this mixture was heated to reflux for 5 hours using a water separator. After cooling the reaction mixture was diluted with ethyl acetate and this organic phase was extracted with water, aq. sodium bicarbonate, brine, dried over sodium sulfate and after filtration evaporated to dryness. The crude product was purified via a Biotage chromatography system (50 g snap KP-Sil column, hexane/0-100% ethylacetate) to obtain 2.47 g (41%) methyl 2-{[bis(4-methoxyphenyl)methyl]carbamoyl}-6-bromoquinoline-4-carboxylate as the desired compound.

¹H-NMR (400 MHz, DMSO d₆) δ (ppm)=3.72 (s, 6H), 4.00 (s, 3H), 6.31-6.35 (m, 1H), 6.88-6.93 (m, 4H), 7.28-7.34 (m, 4H), 8.08 (dd, 1H), 8.21 (d, 1H), 8.54 (s, 1H), 8.95 (d, 1H), 9.38-9.43 (m, 1H).

### Step 2:

A suspension of 500 mg (0.93 mmol) 17.2 g (90.0 mmol) of methyl 2-{[bis(4-methoxyphenyl)methyl]carbamoyl}-6-bromoquinoline-4-carboxylate of step 1) of intermediate 55A), 84.2 mg (1.12 mmol) 2-methoxyethanamine, 85.5 mg (0.093 mmol) tris(dibenzylideneacetone)dipalladium(0), 108 mg (0.187 mmol) Xantphos and 669 mg (2.06 mmol) cesium carbonate in 5.4 mL dioxane was heated up to 80° C. for 3 hours. After cooling to rt the mixture was diluted with ethyl acetate and the organic phase was then extracted with concentrated aq. ammonium chloride, dried over sodium sulfate and after filtration evaporated to dryness. The residue was purified via a Biotage Chromatography system (25 g snap KP-Sil column, hexane/0-100% ethylacetate, then ethyl acetate/0-25% methanol) to obtain 425 mg (69%) methyl 2-{[bis(4-methoxyphenyl)methyl]carbamoyl}-6-[(2-methoxyethyl)amino]quinoline-4-carboxylate as the desired compound.

¹H-NMR (400 MHz, DMSO d₆) δ (ppm)=3.31 (s, 3H), 3.34 (q, 2H), 3.58 (t, 2H), 3.72 (s, 6H), 3.95 (s, 3H), 6.28 (d, 1H), 6.88-6.93 (m, 4H), 6.95 (t, 1H), 7.25-7.31 (m, 4H), 7.40 (dd, 1H), 7.60 (d, 1H), 7.91 (d, 1H), 8.38 (s, 1H), 9.01 (d, 1H).

### Step 3:

To a solution of 425 mg (0.80 mmol) methyl 2-{[bis(4-methoxyphenyl)methyl]carbamoyl}-6-[(2-methoxyethyl)amino]quinoline-4-carboxylate of step 2) of intermediate 55A) in 10 mL dichloromethane was added 0.33 mL (4.32 mmol) trifluoro-acetic acid and 0.64 mL (4.01 mmol) triethylsilane and then this mixture was heated up to 40° C. for 14 hours. Again 0.64 mL (4.01 mmol) triethylsilane and 0.5 mL trifluoro-acetic acid and heated up to 40° C. for 14 hours. And again 0.38 mL (2.41 mmol) triethylsiLane and 0.2 mL trifluoro-acetic acid and heated up to 45° C. for 14 hours. After cooling to rt the mixture was evaporated to dryness and the residue was purified via a Biotage Chromatography system (10 g snap KP-Sil column, hexane/0-100% ethylacetate, then ethyl acetate/0-80% methanol) to obtain 196 mg (81%) methyl 2-carbamoyl-6-[(2-methoxyethyl)amino]quinoline-4-carboxylate as the desired compound.

¹H-NMR (400 MHz, DMSO d₆) δ (ppm)=3.31 (s, 3H), 3.34 (q, 4H), 3.59 (t, 2H), 3.95 (s, 3H), 6.88-6.95 (m, 1H), 7.41 (dd, 1H), 7.59-7.63 (m, 2H), 7.86 (d, 1H), 8.08 (br. s., 1H), 8.40 (s, 1H).

### Step 4:

In analogy to step 4) of intermediate 2A) we got from 196 mg (0.65 mmol) methyl 2-carbamoyl-6-[(2-methoxyethyl)amino]quinoline-4-carboxylate of step 3) of intermediate 55A) 71 mg (36%) of the desired title compound.

¹H-NMR (400 MHz, DMSO d₆) δ (ppm)=3.31 (s, 3H), 3.33 (t, 2H), 3.58 (t, 2H), 7.39 (dd, 1H), 7.58 (br. s., 1H), 7.67 (d, 1H), 7.85 (d, 1H), 8.06 (br. s., 1H), 8.38 (s, 1H).

### Intermediate 56A

### 2-carbamoyl-6-(piperidin-1-yl)quinoline-4-carboxylic acid

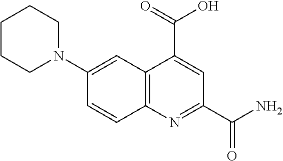 [SMILES: NC(=O)c1cc(C(=O)O)c2cc(N3CCCCC3)ccc2n1]

### Step 1:

In analogy to step 2) of intermediate 55A) we got from 196 mg (0.65 mmol) methyl 2-{[bis(4-methoxyphenyl)methyl]carbamoyl}-6-bromoquinoline-4-carboxylate of step 1) of intermediate 55A) and 70.5 mg (0.83 mmol) piperidine 260 mg (70%) methyl 2-{[bis(4-methoxyphenyl)methyl]carbamoyl}-6-(piperidin-1-yl)quinoline-4-carboxylate as the desired compound.

¹H-NMR (400 MHz, DMSO d₆) δ (ppm)=1.59-1.72 (m, 6H), 3.42-3.48 (m, 4H), 3.74 (s, 6H), 3.98 (s, 3H), 6.31 (d, 1H), 6.89-6.96 (m, 4H), 7.31 (d, 4H), 7.77-7.82 (m, 1H), 7.99 (d, 1H), 8.04 (d, 1H), 8.44 (s, 1H), 9.14 (d, 1H).

### Step 2:

In analogy to step 3) of intermediate 55A) we got from 260 mg (0.48 mmol) methyl 2-{[bis(4-methoxyphenyl)methyl]carbamoyl}-6-(piperidin-1-yl)quinoline-4-carboxylate of step 1) of intermediate 56A) 72 mg (34%) methyl 2-carbamoyl-6-(piperidin-1-yl)quinoline-4-carboxylate as the desired compound.

¹H-NMR (400 MHz, DMSO d₆) δ (ppm)=1.58-1.72 (m, 6H), 3.39-3.47 (m, 4H), 3.97 (s, 3H), 7.68 (br. s., 1H), 7.78 (dd, 1H), 7.94-8.00 (m, 2H), 8.15 (br. s., 1H), 8.44 (s, 1H).

### Step 3:

In analogy to step 4) of intermediate 2A) we got from 72 mg (0.23 mmol) methyl 2-carbamoyl-6-(piperidin-1-yl)quinoline-4-carboxylate of step 3) of intermediate 56A) 24 mg (35%) of the desired title compound.

¹H-NMR (400 MHz, DMSO d₆) δ (ppm)=1.55-1.74 (m, 6H), 3.34-3.46 (m, 4H), 7.68 (br. s., 1H), 7.77 (d, 1H), 7.96 (d, 1H), 8.06 (br. s., 1H), 8.15 (br. s., 1H), 8.43 (s, 1H), 13.65 (br. s., 1H).

### Intermediate 57A

### 2-sulfamoylquinoline-4-carboxylic acid

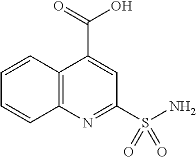 [SMILES: NS(=O)(=O)c1cc(C(=O)O)c2ccccc2n1]

This compound can be prepared starting from commercially available 2,4-dibromoquinoline via the reaction with sodium sulfite to get 4-bromoquinoline-2-sulfonic acid (analogous to US 2008/45568, Chemische Berichte 1920, vol 53, p 1021). Subsequently, the reaction with thionyl chloride followed by ammonia can obtain 4-bromoquinoline-2-sulfonamide. Finally, a carbonylation reaction using molybdenumhexacarbonyl in methanol in the presence of a palladium(II) acetate/1,3-Bis-(diphenylphosphino)-propane/triethylamine as catalyst system following by saponification of the introduced methyl ester with sodium hydroxide can provide the title compound.

### Intermediate 58A

### 2-(methylsulfamoyl)quinoline-4-carboxylic acid

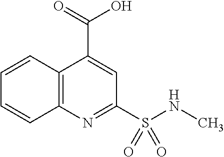 [SMILES: CNS(=O)(=O)c1cc(C(=O)O)c2ccccc2n1]

In analogy to the description of 2-sulfamoylquinoline-4-carboxylic acid (intermediate 57A) using methylamine instead of ammonia can give the title compound.

### Intermediate 59A

### 2-(dimethylsulfamoyl)quinoline-4-carboxylic acid

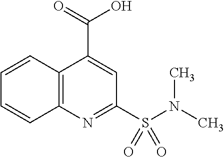 [SMILES: CN(C)S(=O)(=O)c1cc(C(=O)O)c2ccccc2n1]

In analogy to the description of 2-sulfamoylquinoline-4-carboxylic acid (intermediate 57A) using dimethylamine instead of ammonia can give the title compound.

### Intermediate 60A

### 2-[(methylsulfonyl)carbamoyl]quinoline-4-carboxylic acid

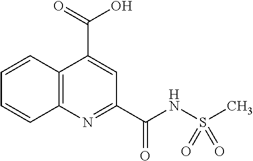 [SMILES: CS(=O)(=O)NC(=O)c1cc(C(=O)O)c2ccccc2n1]

### Step 1:

To a solution of 500 mg (2.17 mmol) methyl 2-carbamoylquinoline-4-carboxylate (step 2 of intermediate 4A) in 8.1 mL THF was given 8.7 mL of a 1M solution of lithium bis(trimethylsilylamide) between −60 and −30° C. (analogous to WO 2012/7877). After stirring for 30 minutes 0.67 mL (8.69 mmol) methanesulfonic acid chloride at −60° C. was added and stirred at −60° C. for 2 hours. The reaction mixture was poured into water. This aqueous phase was extracted three times with ethyl acetate. Then the aqueous phase was evaporated to dryness in vacuum and then stirred in a mixture of methanol and methylene chloride (ratio 1:1). This organic phase was then under evaporation adsorbed on Isolute® HM-N (Biotage) and purified via a Biotage chromatography system (25 g snap KP-Sil column, hexane/80-100% ethylacetate, then ethyl acetate/0-100% methanol) to obtain 320 mg (41%) methyl 2-[(methylsulfonyl)carbamoyl]quinoline-4-carboxylate.

¹H-NMR (400 MHz, DMSO d₆) δ (ppm)=3.15 (s, 3H), 4.03 (s, 3H), 7.83 (ddd, 1H), 7.90-7.96 (m, 1H), 8.27 (d, 1H), 8.53 (s, 1H), 8.70 (d, 1H), 12.14 (br. s., 1H).

### Step 2:

In analogy to step 4) of intermediate 2A) we got from 320 mg (1.04 mmol) methyl 2-[(methylsulfonyl)carbamoyl]quinoline-4-carboxylate of step 1) of intermediate 60A) 120 mg (35%) of the desired title compound.

¹H-NMR (400 MHz, DMSO d₆) δ (ppm)=3.43 (s, 3H), 7.89 (ddd, 1H), 7.98 (ddd, 1H), 8.31 (dd, 1H), 8.45 (s, 1H), 8.80 (dd, 1H).

### Intermediate 1B

### 1-(4-fluorobenzyl)-3,5-dimethyl-4-nitro-1H-pyrazole

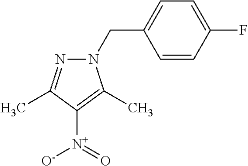 [SMILES: Cc1nn(Cc2ccc(F)cc2)c(C)c1[N+](=O)[O-]]

150 mg (1.06 mmol) 3,5-dimethyl-4-nitro-1H-pyrazole (CAS-No. 14531-55-6) was dissolved in 5 mL acetonitrile and 158 μL (1.28 mmol) 1-(bromomethyl)-4-fluorobenzene and 416 mg (1.28 mmol) cesium carbonate were added. The suspension was stirred at 60° C. for 2 h. Afterwards the reaction mixture was filtered, the filtrate was evaporated and the residue partitioned between ethyl acetate and water. The layers were separated and the aqueous layer was extracted two further times with ethyl acetate. The combined organic layers were washed with brine, dried over sodium sulfate, filtered and evaporated to obtain 259 mg (1.04 mmol, 98%) of the desired title compound after drying.

1H NMR (400 MHz, CDCl₃): δ (ppm)=2.55 (s, 3H), 2.58 (s, 3H), 5.23 (s, 2H), 7.05 (m, 2H), 7.13-7.19 (m, 2H).

### Intermediate 2B

### 1-(3-fluorobenzyl)-3,5-dimethyl-4-nitro-1H-pyrazole

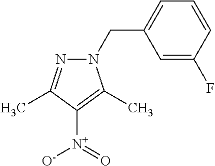 [SMILES: Cc1nn(Cc2cccc(F)c2)c(C)c1[N+](=O)[O-]]

In analogy to intermediate 1B, 400 mg (2.83 mmol) 3,5-dimethyl-4-nitro-1H-pyrazole was heated with 417 μL (3.40 mmol) 1-(bromomethyl)-3-fluorobenzene and 1.11 g (3.40 mmol) cesium carbonate in 10 mL acetonitrile for 2 h at 60° C. Afterwards the reaction mixture was filtered, the filtrate was evaporated, the residue was dissolved in dichloromethane and under evaporation adsorbed on Isolute® HM-N (Biotage). The isolute was given on a Biotage SNAP cartridge (25 g; KP-Sil) preequilibrated with hexane and purified via column chromatography on silica gel (solvent: hexane/0-40% ethyl acetate) to obtain 682 mg (2.74 mmol, 97%) of the desired title compound.

Method 1: Rₜ=1.21 min

MS (ESIpos): m/z=250 (M+H)⁺

### Intermediate 3B

### 1-(2-fluorobenzyl)-3,5-dimethyl-4-nitro-1H-pyrazole

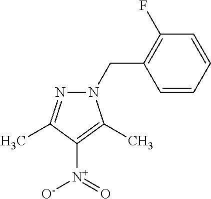 [SMILES: Cc1nn(Cc2ccccc2F)c(C)c1[N+](=O)[O-]]

In analogy to intermediate 1B, 200 mg (1.42 mmol) 3,5-dimethyl-4-nitro-1H-pyrazole was heated with 205 μL (1.70 mmol) 1-(bromomethyl)-2-fluorobenzene and 554 mg (1.70 mmol) cesium carbonate in 5 mL acetonitrile for 2 h at 60° C. Afterwards the reaction mixture was filtered, the filtrate was evaporated, the residue was dissolved in dichloromethane and under evaporation adsorbed on Isolute® HM-N (Biotage). The isolute was given on a Biotage SNAP cartridge (25 g; KP-Sil) preequilibrated with hexane and purified via column chromatography on silica gel (solvent: hexane/0-40% ethyl acetate) to obtain 336 mg (1.35 mmol, 95%) of the desired title compound.

1H NMR (300 MHz, CDCl₃): δ (ppm)=2.54 (s, 3H) 2.62 (s, 3H) 5.31 (s, 2H) 7.01-7.20 (m, 3H) 7.28-7.41 (m, 1H).

### Intermediate 4B

### 1-(3,4-difluorobenzyl)-3,5-dimethyl-4-nitro-1H-pyrazole

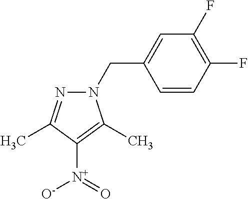 [SMILES: Cc1nn(Cc2ccc(F)c(F)c2)c(C)c1[N+](=O)[O-]]

In analogy to intermediate 1B, 400 mg (2.83 mmol) 3,5-dimethyl-4-nitro-1H-pyrazole was heated with 435 μL (3.40 mmol) 4-(bromomethyl)-1,2-difluorobenzene and 1.11 g (3.40 mmol) cesium carbonate in 10 mL acetonitrile for 2 h at 60° C. Afterwards the reaction mixture was filtered, the filtrate was evaporated to obtain 726 mg (2.72 mmol, 96%) of the desired title compound after drying.

Method 1: Rₜ=1.23 min

MS (ESIpos): m/z=268 (M+H)⁺.

### Intermediate 5B

### 1-(2,4-difluorobenzyl)-3,5-dimethyl-4-nitro-1H-pyrazole

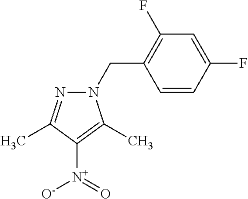 [SMILES: Cc1nn(Cc2ccc(F)cc2F)c(C)c1[N+](=O)[O-]]

In analogy to intermediate 1B, 400 mg (2.83 mmol) 3,5-dimethyl-4-nitro-1H-pyrazole was heated with 436 μL (3.40 mmol) 1-(bromomethyl)-2,4-difluorobenzene and 1.11 g (3.40 mmol) cesium carbonate in 10 mL acetonitrile for 2 h at 60° C. Afterwards, the reaction mixture was filtered, and the filtrate was evaporated to obtain 716 mg (2.68 mmol, 95%) of the desired title compound after drying.

Method 1: Rₜ=1.23 min

MS (ESIpos): m/z=268 (M+H)⁺.

### Intermediate 6B

### 1-(2,6-difluorobenzyl)-3,5-dimethyl-4-nitro-1H-pyrazole

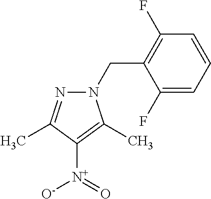 [SMILES: Cc1nn(Cc2c(F)cccc2F)c(C)c1[N+](=O)[O-]]

In analogy to intermediate 1B, 400 mg (2.83 mmol) 3,5-dimethyl-4-nitro-1H-pyrazole was heated with 436 μL (3.40 mmol) 2-(bromomethyl)-1,3-difluorobenzene and 1.11 g (3.40 mmol) cesium carbonate in 10 mL acetonitrile for 2 h at 60° C. Afterwards the reaction mixture was filtered, the filtrate was evaporated to obtain 719 mg (2.69 mmol, 95%) of the desired title compound after drying.

Method 1: Rₜ=1.19 min

MS (ESIpos): m/z=268 (M+H)⁺.

### Intermediate 7B

### 3,5-dimethyl-4-nitro-1-(2,4,6-trifluorobenzyl)-1H-pyrazole

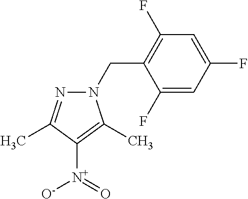 [SMILES: Cc1nn(Cc2c(F)cc(F)cc2F)c(C)c1[N+](=O)[O-]]

In analogy to intermediate 1B, 400 mg (2.83 mmol) 3,5-dimethyl-4-nitro-1H-pyrazole was heated with 446 μL (3.40 mmol) 2-(bromomethyl)-1,3,5-trifluorobenzene and 1.11 g (3.40 mmol) cesium carbonate in 10 mL acetonitrile for 2 h at 60° C. Afterwards the reaction mixture was filtered, the filtrate was evaporated to obtain 708 mg (2.48 mmol, 88%) of the desired title compound after drying.

Method 1: Rₜ=1.18 min

MS (ESIpos): m/z=286 (M+H)⁺.

### Intermediate 8B

### 4-[(3,5-dimethyl-4-nitro-1H-pyrazol-1-yl)methyl]benzonitrile

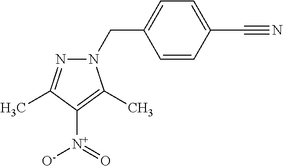 [SMILES: Cc1nn(Cc2ccc(C#N)cc2)c(C)c1[N+](=O)[O-]]

In analogy to intermediate 1B, 6.0 g (42.5 mmol) 3,5-dimethyl-4-nitro-1H-pyrazole and 10.0 g (51.0 mmol) 4-(bromomethyl)-benzonitrile were reacted to give after purification of the crude product via a Biotage chromatography system (100 g snap KP-Sil column, hexane/30-100% ethyl acetate) 9.56 g (88%) of the desired title compound.

¹H-NMR (400 MHz, DMSO d₆) δ (ppm)=2.40 (s, 3H), 2.56 (s, 3H), 5.48 (s, 2H), 7.35 (d, 2H), 7.82 (d, 2H).

### Intermediate 9B

### 3-[(3,5-dimethyl-4-nitro-1H-pyrazol-1-yl)methyl]benzonitrile

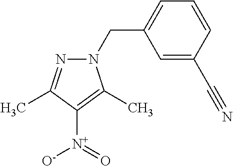 [SMILES: Cc1nn(Cc2cccc(C#N)c2)c(C)c1[N+](=O)[O-]]

In analogy to intermediate 1B, 400 mg (2.83 mmol) 3,5-dimethyl-4-nitro-1H-pyrazole was heated with 666 mg (3.40 mmol) 3-(bromomethyl)benzonitrile and 1.11 g (3.40 mmol) cesium carbonate in 10 mL acetonitrile for 2 h at 60° C. Afterwards the reaction mixture was filtered, the filtrate was evaporated, the residue was dissolved in dichloromethane and under evaporation adsorbed on Isolute® HM-N (Biotage). The isolute was given on a Biotage SNAP cartridge (25 g; KP-Sil) preequilibrated with hexane and purified via column chromatography on silica gel (solvent: hexane/0-50% ethyl acetate) to obtain 705 mg (2.75 mmol, 97%) of the desired title compound after drying.

1H NMR (400 MHz, CDCl₃): δ (ppm)=2.56 (s, 3H), 2.59 (s, 3H), 5.29 (s, 2H), 7.40 (d, 1H), 7.44-7.53 (m, 2H), 7.64 (d, 1H).

### Intermediate 10B

### 2-[(3,5-dimethyl-4-nitro-1H-pyrazol-1-yl)methyl]benzonitrile

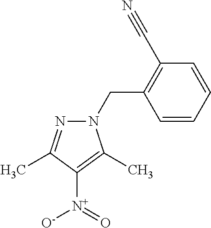 [SMILES: Cc1nn(Cc2ccccc2C#N)c(C)c1[N+](=O)[O-]]

In analogy to intermediate 1B), 1.5 g (10.6 mmol) 3,5-dimethyl-4-nitro-1H-pyrazole and 2.5 g (12.8 mmol) 2-(bromomethyl)-benzonitrile were reacted to give after purification of the crude product via a Biotage chromatography system (50 g snap KP-Sil column, hexane/15-100% ethyl acetate) 2.48 g (89%) of the desired title compound.

¹H-NMR (400 MHz, DMSO d₆) δ (ppm)=2.37 (s, 3H), 2.64 (s, 3H), 5.53 (s, 2H), 7.22 (d, 1H), 7.49-7.57 (m, 1H), 7.69 (td, 1H), 7.89 (dd, 1H).

### Intermediate 11B

### 1-(4-methoxybenzyl)-3,5-dimethyl-4-nitro-1H-pyrazole

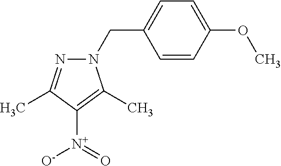

In analogy to intermediate 1B), 1.5 g (10.6 mmol) 3,5-dimethyl-4-nitro-1H-pyrazole and 2.56 g (12.8 mmol) 1-(bromomethyl)-4-methoxybenzene were reacted to give after purification of the crude product via a Biotage chromatography system (50 g snap KP-Sil column, hexane/30-100% ethyl acetate) 2.41 g (85%) of the desired title compound.

¹H-NMR (400 MHz, DMSO d₆) δ (ppm)=2.39 (s, 3H), 2.57 (s, 3H), 3.72 (s, 3H), 5.26 (s, 2H), 6.87-6.93 (m, 2H), 7.14-7.20 (m, 2H).

### Intermediate 12B

### 1-(3-methoxybenzyl)-3,5-dimethyl-4-nitro-1H-pyrazole

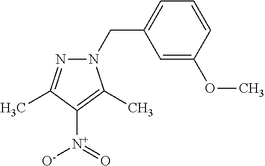

In analogy to intermediate 1B, 200 mg (1.42 mmol) 3,5-dimethyl-4-nitro-1H-pyrazole was heated with 341 mg (1.70 mmol) 1-(bromomethyl)-3-methoxybenzene and 554 mg (1.70 mmol) cesium carbonate in 10 mL acetonitrile for 2 h at 60° C. Afterwards the reaction mixture was filtered, the filtrate was evaporated, the residue was dissolved in dichloromethane and under evaporation adsorbed on Isolute® HM-N (Biotage). The isolute was given on a Biotage SNAP cartridge (25 g; KP-Sil) preequilibrated with hexane and purified via column chromatography on silica gel (solvent: hexane/0-50% ethyl acetate) to obtain 368 mg (1.41 mmol, 99%) of the desired title compound after drying.

Method 1: Rₜ=1.19 min

MS (ESIpos): m/z=262 (M+H)⁺.

### Intermediate 13B

### 3,5-dimethyl-1-(4-methylbenzyl)-4-nitro-1H-pyrazole

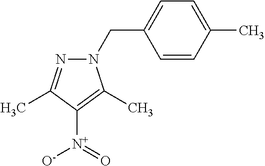

In analogy to intermediate 1B, 400 mg (2.83 mmol) 3,5-dimethyl-4-nitro-1H-pyrazole was heated with 629 mg (3.40 mmol) 1-(bromomethyl)-4-methylbenzene and 1.11 g (3.40 mmol) cesium carbonate in 10 mL acetonitrile for 2 h at 60° C. Afterwards the reaction mixture was filtered, the filtrate was evaporated to obtain 686 mg (2.54 mmol, 90%) of the desired title compound after drying.

Method 1: Rₜ=1.28 min

MS (ESIpos): m/z=246 (M+H)⁺.

### Intermediate 14B

### 3,5-dimethyl-1-(3-methylbenzyl)-4-nitro-1H-pyrazole

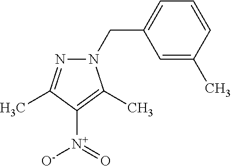

In analogy to intermediate 1B, 400 mg (2.83 mmol) 3,5-dimethyl-4-nitro-1H-pyrazole was heated with 629 mg (3.40 mmol) 1-(bromomethyl)-3-methylbenzene and 1.11 g (3.40 mmol) cesium carbonate in 10 mL acetonitrile for 2 h at 60° C. Afterwards the reaction mixture was filtered, the filtrate was evaporated to obtain 681 mg (2.50 mmol, 88%) of the desired title compound after drying.

Method 1: Rₜ=1.27 min

MS (ESIpos): m/z=246 (M+H)⁺.

### Intermediate 15B

### 3,5-dimethyl-1-(2-methylbenzyl)-4-nitro-1H-pyrazole

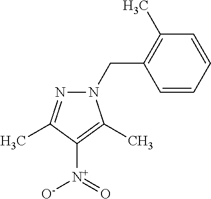 [SMILES: Cc1ccccc1Cn1nc(C)c([N+](=O)[O-])c1C]

In analogy to intermediate 1B, 400 mg (2.83 mmol) 3,5-dimethyl-4-nitro-1H-pyrazole was heated with 629 mg (3.40 mmol) 1-(bromomethyl)-2-methylbenzene and 1.11 g (3.40 mmol) cesium carbonate in 10 mL acetonitrile for 2 h at 60° C. Afterwards the reaction mixture was filtered, the filtrate was evaporated to obtain 695 mg (2.47 mmol, 87%) of the desired title compound after drying.

Method 1: Rₜ=1.26 min

MS (ESIpos): m/z=246 (M+H)⁺.

### Intermediate 16B

### 4-[(3,5-dimethyl-4-nitro-1H-pyrazol-1-yl)methyl]pyridine

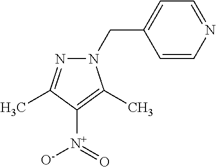 [SMILES: Cc1nn(Cc2ccncc2)c(C)c1[N+](=O)[O-]]

In analogy to intermediate 1B), 1.5 g (10.6 mmol) 3,5-dimethyl-4-nitro-1H-pyrazole and 3.23 g (12.8 mmol) 4-(bromomethyl)pyridine hydrobromide salt were reacted to give after purification of the crude product via a Biotage chromatography system (25 g snap KP-Sil column, hexane/0-100% ethyl acetate, then ethyl acetate/0-45% methanol) 1.576 g (62%) of the desired title compound.

¹H-NMR (300 MHz, DMSO d₆) δ (ppm)=2.41 (s, 3H), 2.56 (s, 3H), 5.44 (s, 2H), 7.08-7.15 (m, 2H), 8.49-8.56 (m, 2H).

### Intermediate 17B

### 3-[(3,5-dimethyl-4-nitro-1H-pyrazol-1-yl)methyl]pyridine

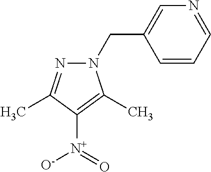 [SMILES: Cc1nn(Cc2cccnc2)c(C)c1[N+](=O)[O-]]

In analogy to intermediate 1B), 1.8 g (12.8 mmol) 3,5-dimethyl-4-nitro-1H-pyrazole and 3.87 g (15.3 mmol) 3-(bromomethyl)pyridine hydrobromide salt were reacted to give after purification of the crude product via a Biotage chromatography system (50 g snap KP-Sil column, hexane/0-100% ethyl acetate, then ethyl acetate/0-45% methanol) 2.24 g (68%) of the desired title compound.

¹H-NMR (300 MHz, DMSO d₆) δ (ppm)=2.38 (s, 3H), 2.62 (s, 3H), 5.41 (s, 2H), 7.38 (dd, 1H), 7.61 (dt, 1H), 8.48-8.53 (m, 2H).

### Intermediate 18B

### 2-[(3,5-dimethyl-4-nitro-1H-pyrazol-1-yl)methyl]pyridine

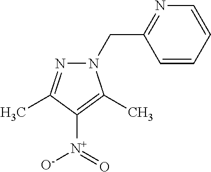 [SMILES: Cc1nn(Cc2ccccn2)c(C)c1[N+](=O)[O-]]

In analogy to intermediate 1B, 300 mg (2.13 mmol) 3,5-dimethyl-4-nitro-1H-pyrazole was heated with 645 mg (2.55 mmol) 2-(bromomethyl)pyridine hydrobromide and 1.52 g (4.68 mmol) cesium carbonate in 10 mL acetonitrile for 2 h at 60° C. Afterwards the reaction mixture was filtered, the filtrate was evaporated, the residue was dissolved in dichloromethane and under evaporation adsorbed on Isolute® HM-N (Biotage). The isolute was given on a Biotage SNAP cartridge (25 g; KP-Sil) preequilibrated with hexane and purified via column chromatography on silica gel (solvent: hexane/0-75% ethyl acetate) to obtain 467 mg (2.01 mmol, 95%) of the desired title compound after drying.

1H NMR (300 MHz, CDCl₃): δ (ppm)=2.55 (s, 3H), 2.64 (s, 3H), 5.38 (s, 2H), 7.08 (d, 1H), 7.21-7.26 (m, 1H), 7.69 (td, 1H), 8.58 (d, 1H).

### Intermediate 19B

### 3,5-dimethyl-4-nitro-1-[4-(trifluoromethoxy)benzyl]-1H-pyrazole

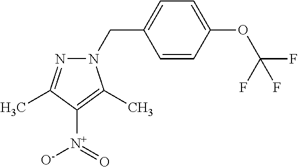 [SMILES: Cc1nn(Cc2ccc(OC(F)(F)F)cc2)c(C)c1[N+](=O)[O-]]

In analogy to intermediate 1B, 400 mg (2.83 mmol) 3,5-dimethyl-4-nitro-1H-pyrazole was heated with 544 μL (3.40 mmol) 1-(bromomethyl)-4-(trifluoromethoxy)benzene and 1.11 g (3.40 mmol) cesium carbonate in 10 mL acetonitrile for 2 h at 60° C. Afterwards the reaction mixture was filtered, the filtrate was evaporated to obtain 891 mg (2.83 mmol, 99%) of the desired title compound after drying.

Method 1: Rₜ=1.35 min

MS (ESIpos): m/z=316 (M+H)⁺.

### Intermediate 20B

### 3,5-dimethyl-4-nitro-1-[3-(trifluoromethoxy)benzyl]-1H-pyrazole

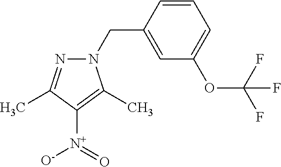 [SMILES: Cc1nn(Cc2cccc(OC(F)(F)F)c2)c(C)c1[N+](=O)[O-]]

In analogy to intermediate 1B, 400 mg (2.83 mmol) 3,5-dimethyl-4-nitro-1H-pyrazole was heated with 551 μL (3.40 mmol) 1-(bromomethyl)-3-(trifluoromethoxy)benzene and 1.11 g (3.40 mmol) cesium carbonate in 10 mL acetonitrile for 2 h at 60° C. Afterwards the reaction mixture was filtered, the filtrate was evaporated to obtain 890 mg (2.31 mmol, 82%) of the desired title compound after drying.

Method 1: Rₜ=1.35 min

MS (ESIpos): m/z=316 (M+H)⁺.

### Intermediate 21B

### 3,5-dimethyl-4-nitro-1-[2-(trifluoromethoxy)benzyl]-1H-pyrazole

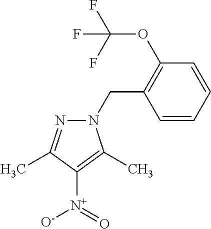 [SMILES: Cc1nn(Cc2ccccc2OC(F)(F)F)c(C)c1[N+](=O)[O-]]

In analogy to intermediate 1B, 400 mg (2.83 mmol) 3,5-dimethyl-4-nitro-1H-pyrazole was heated with 548 μL (3.40 mmol) 1-(bromomethyl)-2-(trifluoromethoxy)benzene and 1.11 g (3.40 mmol) cesium carbonate in 10 mL acetonitrile for 2 h at 60° C. Afterwards the reaction mixture was filtered, the filtrate was evaporated to obtain 893 mg (2.83 mmol, 99%) of the desired title compound after drying.

Method 1: Rₜ=1.35 min

MS (ESIpos): m/z=316 (M+H)⁺.

### Intermediate 22B

### 3,5-dimethyl-4-nitro-1-[4-(trifluoromethyl)benzyl]-1H-pyrazole

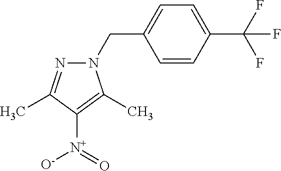 [SMILES: Cc1nn(Cc2ccc(C(F)(F)F)cc2)c(C)c1[N+](=O)[O-]]

In analogy to intermediate 1B, 400 mg (2.83 mmol) 3,5-dimethyl-4-nitro-1H-pyrazole was heated with 813 mg (3.40 mmol) 1-(bromomethyl)-4-(trifluoromethyl)benzene and 1.10 g (3.40 mmol) cesium carbonate in 10 mL acetonitrile for 2 h at 60° C. Afterwards the reaction mixture was filtered, the filtrate was evaporated, the residue was dissolved in dichloromethane and under evaporation adsorbed on Isolute® HM-N (Biotage). The isolute was given on a Biotage SNAP cartridge (25 g; KP-Sil) preequilibrated with hexane and purified via column chromatography on silica gel (solvent: hexane-ethyl acetate) to obtain 790 mg (2.64 mmol, 93%) of the desired title compound after drying.

1H NMR (300 MHz, CDCl₃): δ (ppm)=2.53-2.57 (m, 3H), 2.58 (s, 3H), 5.33 (s, 2H), 7.27 (d, 2H), 7.63 (d, 2H).

### Intermediate 23B

### 1-(3,4-difluorobenzyl)-5-methyl-4-nitro-3-(trifluoromethyl)-1H-pyrazole

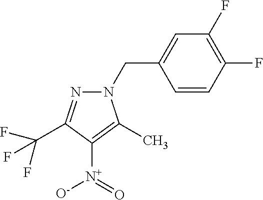 [SMILES: Cc1c([N+](=O)[O-])c(C(F)(F)F)nn1Cc1ccc(F)c(F)c1]

In analogy to intermediate 1B), 1.2 g (6.15 mmol) 5-methyl-4-nitro-3-(trifluoromethyl)-1H-pyrazole and 1.53 g (7.38 mmol) 4-(bromomethyl)-1,2-difluorobenzene were reacted to give after purification of the crude product via a Biotage chromatography system (25 g snap KP-Sil column, hexane/0-70% ethyl acetate) 1.76 g (85%) of the desired title compound.

¹H-NMR (300 MHz, DMSO d₆) δ (ppm)=2.65 (s, 3H), 5.53 (s, 2H), 7.12 (ddd, 1H), 7.38 (ddd, 1H), 7.45 (dt, 1H).

### Intermediate 24B

### 1-(2,4-difluorobenzyl)-5-methyl-4-nitro-3-(trifluoromethyl)-1H-pyrazole

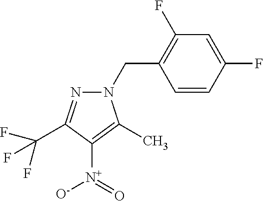 [SMILES: Cc1c([N+](=O)[O-])c(C(F)(F)F)nn1Cc1ccc(F)cc1F]

In analogy to intermediate 1B), 1.2 g (6.15 mmol) 5-methyl-4-nitro-3-(trifluoromethyl)-1H-pyrazole and 1.53 g (7.38 mmol) 1-(bromomethyl)-2,4-difluorobenzene were reacted to give after purification of the crude product via a Biotage chromatography system (25 g snap KP-Sil column, hexane/0-70% ethyl acetate) 1.73 g (83%) of the desired title compound.

¹H-NMR (300 MHz, DMSO d₆) δ (ppm)=2.68 (s, 3H), 5.54 (s, 2H), 7.12 (tdd, 1H), 7.27-7.44 (m, 2H).

### Intermediate 25B

### 1-(4-fluorobenzyl)-5-methyl-4-nitro-3-(trifluoromethyl)-1H-pyrazole

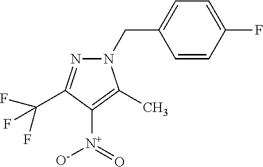 [SMILES: Cc1c([N+](=O)[O-])c(C(F)(F)F)nn1Cc1ccc(F)cc1]

In analogy to intermediate 1B), 3.51 g (18.0 mmol) 5-methyl-4-nitro-3-(trifluoromethyl)-1H-pyrazole and 4.08 g (21.6 mmol) 1-(bromomethyl)-4-fluorobenzene were reacted to give after purification of the crude product via a Biotage chromatography system (25 g snap KP-Sil column, hexane/0-70% ethyl acetate) 4.21 g (73%) of the desired title compound.

¹H-NMR (400 MHz, DMSO d₆) δ (ppm)=2.66 (s, 3H), 5.53 (s, 2H), 7.17-7.24 (m, 2H), 7.30-7.37 (m, 2H).

### Intermediate 26B

### 4-{[5-methyl-4-nitro-3-(trifluoromethyl)-1H-pyrazol-1-yl]methyl}benzonitrile

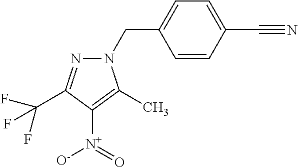 [SMILES: Cc1c([N+](=O)[O-])c(C(F)(F)F)nn1Cc1ccc(C#N)cc1]

In analogy to intermediate 1B), 4.30 g (22.0 mmol) 5-methyl-4-nitro-3-(trifluoromethyl)-1H-pyrazole and 5.19 g (26.5 mmol) 4-(bromomethyl)benzonitrile were reacted to give after purification of the crude product via a Biotage chromatography system (100 g snap KP-Sil column, hexane/0-70% ethyl acetate) 6.30 g (88%) of the desired title compound.

¹H-NMR (300 MHz, DMSO d₆) δ (ppm)=2.63 (s, 3H), 5.67 (s, 2H), 7.38-7.44 (m, 2H), 7.83-7.88 (m, 2H).

### Intermediate 27B

### 1-(4-chlorobenzyl)-3,5-dimethyl-4-nitro-1H-pyrazole

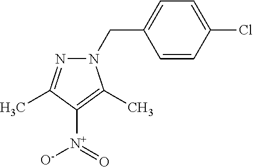 [SMILES: Cc1nn(Cc2ccc(Cl)cc2)c(C)c1[N+](=O)[O-]]

In analogy to intermediate 1B), 1.72 g (12.2 mmol) 3,5-dimethyl-4-nitro-1H-pyrazole and 3.00 g (14.6 mmol) 1-(bromomethyl)-4-chlorobenzene were reacted to give after purification of the crude product via a Biotage chromatography system (50 g snap KP-Sil column, hexane/0-100% ethyl acetate, then ethyl acetate/0-45% methanol) 3.5 g (101%, purity: 94%) of the desired title compound.

¹H-NMR (300 MHz, DMSO d₆) δ (ppm)=2.39 (s, 3H), 2.57 (s, 3H), 5.36 (s, 2H), 7.18-7.25 (m, 2H), 7.38-7.44 (m, 2H).

### Intermediate 28B

### 1-(3-chloro-4-fluorobenzyl)-3,5-dimethyl-4-nitro-1H-pyrazole

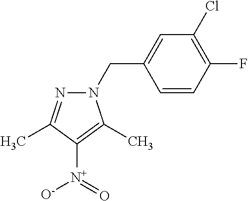 [SMILES: Cc1nn(Cc2ccc(F)c(Cl)c2)c(C)c1[N+](=O)[O-]]

In analogy to intermediate 1B), 1.58 g (11.2 mmol) 3,5-dimethyl-4-nitro-1H-pyrazole and 3.00 g (13.4 mmol) 4-(bromomethyl)-2-chloro-1-fluorobenzene were reacted to give after purification of the crude product via a Biotage chromatography system (50 g snap KP-Sil column, hexane/0-100% ethyl acetate, then ethyl acetate/0-45% methanol) 3.16 g (85%) of the desired title compound.

¹H-NMR (300 MHz, DMSO d₆) δ (ppm)=2.39 (s, 3H), 2.59 (s, 3H), 5.35 (s, 2H), 7.21 (ddd, 1H), 7.39 (t, 1H), 7.49 (dd, 1H).

### Intermediate 29B

### 5-[(3,5-dimethyl-4-nitro-1H-pyrazol-1-yl)methyl]pyridine-2-carbonitrile

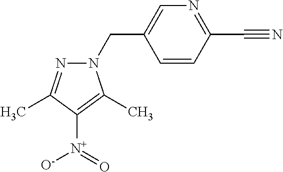 [SMILES: Cc1nn(Cc2ccc(C#N)nc2)c(C)c1[N+](=O)[O-]]

In analogy to intermediate 1B), 2.98 g (21.1 mmol) 3,5-dimethyl-4-nitro-1H-pyrazole and 5.00 g (25.4 mmol) 5-(bromomethyl)pyridine-2-carbonitrile were reacted to give after purification of the crude product via a Biotage chromatography system (100 g snap KP-Sil column, hexane/20-100% ethyl acetate) 3.69 g (64%) of the desired title compound.

¹H-NMR (300 MHz, DMSO d₆) δ (ppm)=2.39 (s, 3H), 2.61 (s, 3H), 5.53 (s, 2H), 7.81 (dd, 1H), 8.02 (dd, 1H), 8.66 (d, 1H).

### Intermediate 30B

### 1-benzyl-3,5-dimethyl-4-nitro-1H-pyrazole

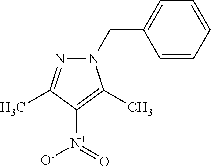 [SMILES: Cc1nn(Cc2ccccc2)c(C)c1[N+](=O)[O-]]

### Step 1: 1-benzyl-3,5-dimethyl-1H-pyrazole

In analogy to intermediate 1B, 500 mg (5.20 mmol) 3,5-dimethyl-1H-pyrazole was heated with 741 μL (6.24 mmol) benzyl bromide and 2.03 g (6.24 mmol) cesium carbonate in 10 mL acetonitrile for 2 h at 60° C. Afterwards the reaction mixture was filtered, the filtrate was evaporated, the residue was dissolved in dichloromethane and under evaporation adsorbed on Isolute® HM-N (Biotage). The isolute was given on a Biotage SNAP cartridge (25 g; KP-Sil) preequilibrated with hexane and purified via column chromatography on silica gel (solvent: hexane-ethyl acetate) to obtain 508 mg (2.73 mmol, 52%) 1-benzyl-3,5-dimethyl-1H-pyrazole after drying.

1H NMR (400 MHz, CDCl₃): δ (ppm)=2.15 (s, 3H), 2.26 (s, 3H), 5.23 (s, 2H), 5.86 (s, 1H), 7.08 (d, 2H), 7.22-7.26 (m, 1H), 7.28-7.35 (m, 2H).

### Step 2: 1-benzyl-3,5-dimethyl-4-nitro-1H-pyrazole

500 mg (2.68 mmol) 1-benzyl-3,5-dimethyl-1H-pyrazole was dissolved in 4 mL acetic anhydride and a solution of 200 μL (3.14 mmol) concentrated nitric acid in 1 mL acetic anhydride was added slowly at 0° C. The stirring was continued for 24 h at 25° C. Afterwards the reaction mixture was carefully poured in a solution of saturated sodium bicarbonate solution and the aqueous layer was extracted with ethyl acetate. The organic layer was washed with brine, dried over sodium sulfate, filtered and evaporated. The residue was dissolved in dichloromethane and under evaporation adsorbed on Isolute® HM-N (Biotage). The isolute was given on a Biotage SNAP cartridge (25 g; KP-Sil) preequilibrated with hexane and purified via column chromatography on silica gel (solvent: hexane-ethyl acetate) to obtain 610 mg (2.64 mmol, 98%) of the desired title compound after drying.

Method 1: Rₜ=1.18 min

MS (ESIpos): m/z=232 (M+H)⁺.

### Intermediate 31B

### methyl 4-[(3,5-dimethyl-4-nitro-1H-pyrazol-1-yl)methyl]benzoate

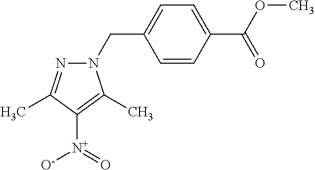

In analogy to intermediate 1B), 10.3 g (72.8 mmol) 3,5-dimethyl-4-nitro-1H-pyrazole and 20.0 g (87.3 mmol) methyl 4-(bromomethyl)benzoate were reacted to give after two subsequent purification of the crude product via a Biotage chromatography system (100 and 50 g snap KP-Sil column, hexane/0-100% ethyl acetate) 18.3 g (82%) of the desired title compound.

¹H-NMR (300 MHz, DMSO d₆) δ (ppm)=2.40 (s, 3H), 2.56 (s, 3H), 3.83 (s, 3H), 5.47 (s, 2H), 7.25-7.34 (m, 2H), 7.89-7.96 (m, 2H).

### Intermediate 32B

### methyl {4-[(3,5-dimethyl-4-nitro-1H-pyrazol-1-yl)methyl]phenyl}acetate

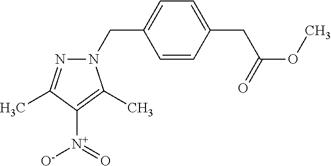

In analogy to intermediate 1B), 2.42 g (17.1 mmol) 3,5-dimethyl-4-nitro-1H-pyrazole and 5.00 g (20.6 mmol) methyl [4-(bromomethyl)phenyl]acetate were reacted to give after purification of the crude product via a Biotage chromatography system (50 g snap KP-Sil column, hexane/0-100% ethyl acetate) 3.81 g (55%) of the desired title compound.

¹H-NMR (400 MHz, DMSO d₆) δ (ppm)=2.39 (s, 3H), 2.58 (s, 3H), 3.59 (s, 3H), 3.65 (s, 2H), 5.33 (s, 2H), 7.13-7.17 (m, 2H), 7.22-7.26 (m, 2H).

### Intermediate 33B

### 1-(cyclohexylmethyl)-3,5-dimethyl-4-nitro-1H-pyrazole

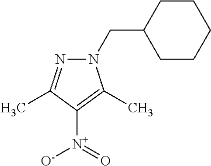 [SMILES: Cc1nn(CC2CCCCC2)c(C)c1[N+](=O)[O-]]

In analogy to intermediate 1B), 3.32 g (23.5 mmol) 3,5-dimethyl-4-nitro-1H-pyrazole and 5.00 g (28.2 mmol) (bromomethyl)cyclohexane were reacted to give after purification of the crude product via a Biotage chromatography system (100 g snap KP-Sil column, hexane/0-100% ethyl acetate) 4.76 g (81%) of the desired title compound.

¹H-NMR (400 MHz, DMSO d₆) δ (ppm)=0.89-1.04 (m, 2H), 1.06-1.22 (m, 3H), 1.50 (d, 2H), 1.55-1.69 (m, 3H), 1.72-1.85 (m, 1H), 2.38 (s, 3H), 2.55 (s, 3H), 3.90 (d, 2H).

### Intermediate 34B

### 1-[(5-chloro-2-thienyl)methyl]-3,5-dimethyl-4-nitro-1H-pyrazole

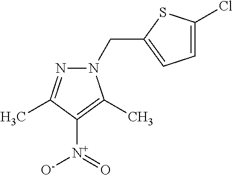 [SMILES: Cc1nn(Cc2ccc(Cl)s2)c(C)c1[N+](=O)[O-]]

In analogy to intermediate 1B), 1.41 g (9.98 mmol) 3,5-dimethyl-4-nitro-1H-pyrazole and 2.00 g (12.0 mmol) 2-chloro-5-(chloromethyl)thiophene were reacted to give after purification of the crude product via a Biotage chromatography system (25 g snap KP-Sil column, hexane/0-100% ethyl acetate, then ethyl acetate/0-50% methanol) 2.51 g (88%) of the desired title compound.

¹H-NMR (400 MHz, DMSO d₆) δ (ppm)=2.39 (s, 3H), 2.62 (s, 3H), 5.49 (s, 2H), 6.98-7.01 (m, 1H), 7.01-7.04 (m, 1H).

### Intermediate 35B

### 3,5-dimethyl-1-[(1-methyl-1H-pyrazol-3-yl)methyl]-4-nitro-1H-pyrazole

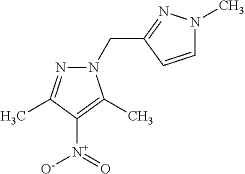

In analogy to intermediate 1B), 0.90 g (6.38 mmol) 3,5-dimethyl-4-nitro-1H-pyrazole and 1.00 g (7.66 mmol) 3-(chloromethyl)-1-methyl-1H-pyrazole were reacted to give after purification of the crude product via a Biotage chromatography system (25 g snap KP-Sil column, hexane/0-100% ethyl acetate, then ethyl acetate/0-50% methanol) 1.28 g (83%) of the desired title compound.

¹H-NMR (300 MHz, DMSO d₆) δ (ppm)=2.37 (s, 3H), 2.62 (s, 3H), 3.77 (s, 3H), 5.24 (s, 2H), 6.13 (d, 1H), 7.61 (d, 1H).

### Intermediate 36B

### 2-[(3,5-dimethyl-4-nitro-1H-pyrazol-1-yl)methyl]-3-methylpyridine

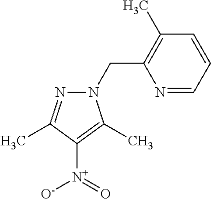 [SMILES: Cc1cccnc1Cn1nc(C)c([N+](=O)[O-])c1C]

In analogy to intermediate 1B, 200 mg (1.41 mmol) 3,5-dimethyl-4-nitro-1H-pyrazole was heated with 316 mg (1.70 mmol) 2-(bromomethyl)-6-methylpyridine hydrobromide and 554 mg (1.70 mmol) cesium carbonate in 10 mL acetonitrile for 2 h at 60° C. Afterwards the reaction mixture was filtered, the filtrate was evaporated, the residue was dissolved in dichloromethane and under evaporation adsorbed on Isolute® HM-N (Biotage). The isolute was given on a Biotage SNAP cartridge (25 g; KP-Sil) preequilibrated with hexane and purified via column chromatography on silica gel (solvent: hexane/0-75% ethyl acetate) to obtain 339 mg (1.38 mmol, 97%) of the desired title compound after drying.

1H NMR (300 MHz, CDCl₃): δ (ppm)=2.47-2.60 (m, 6H), 2.64 (s, 3H), 5.34 (s, 2H), 6.78 (d, 1H), 7.09 (d, 1H), 7.55 (t, 1H).

### Intermediate 37B

### 3,5-dimethyl-1-[4-(methylsulfonyl)benzyl]-4-nitro-1H-pyrazole

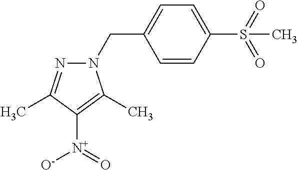

In analogy to intermediate 1B), 0.66 g (4.67 mmol) 3,5-dimethyl-4-nitro-1H-pyrazole and 1.40 g (5.62 mmol) 4-(bromomethyl)phenyl methyl sulfone were reacted to give after purification of the crude product via a Biotage chromatography system (25 g snap KP-Sil column, hexane/0-100% ethyl acetate, then ethyl acetate/0-15% methanol) 0.71 g (48%) of the desired title compound.

¹H-NMR (300 MHz, DMSO d₆) δ (ppm)=2.40 (s, 3H), 2.59 (s, 3H), 3.19 (s, 3H), 5.50 (s, 2H), 7.40-7.46 (m, 2H), 7.87-7.93 (m, 2H).

### Intermediate 38B

### 4-[(3,5-dimethyl-4-nitro-1H-pyrazol-1-yl)methyl]-2-fluorobenzonitrile

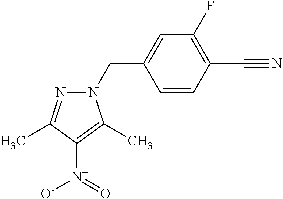 [SMILES: Cc1nn(Cc2ccc(C#N)c(F)c2)c(C)c1[N+](=O)[O-]]

In analogy to intermediate 1B), 0.66 g (4.67 mmol) 3,5-dimethyl-4-nitro-1H-pyrazole and 1.00 g (5.62 mmol) 4-(bromomethyl)-2-fluorobenzonitrile were reacted to give after purification of the crude product via a Biotage chromatography system (25 g snap KP-Sil column, hexane/30-100% ethyl acetate) 1.14 g (86%) of the desired title compound.

¹H-NMR (300 MHz, DMSO d₆) δ (ppm)=2.37 (s, 3H), 2.61 (s, 3H), 5.49 (s, 2H), 7.30 (t, 1H), 7.68 (dd, 1H), 7.91 (dd, 1H).

### Intermediate 39B

### 3-[(3,5-dimethyl-4-nitro-1H-pyrazol-1-yl)methyl]pyridine-2-carbonitrile

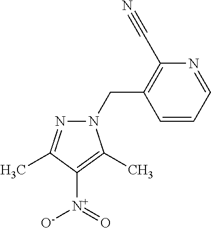 [SMILES: Cc1nn(Cc2cccnc2C#N)c(C)c1[N+](=O)[O-]]

In analogy to intermediate 1B), 1.49 g (10.6 mmol) 3,5-dimethyl-4-nitro-1H-pyrazole and 2.50 g (12.7 mmol) 3-(bromomethyl)pyridine-2-carbonitrile were reacted to give after purification of the crude product via a Biotage chromatography system (50 g snap KP-Sil column, hexane/0-100% ethyl acetate, then ethyl acetate/0-15% methanol) 2.36 g (82%) of the desired title compound.

¹H-NMR (400 MHz, DMSO d₆) δ (ppm)=2.37 (s, 3H), 2.67 (s, 3H), 5.58 (s, 2H), 7.71-7.74 (m, 2H), 8.71 (t, 1H).

### Intermediate 40B

### 2-[(3,5-dimethyl-4-nitro-1H-pyrazol-1-yl)methyl]nicotinonitrile

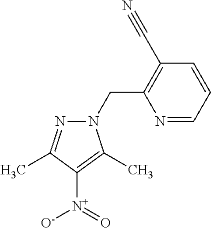 [SMILES: Cc1nn(Cc2ncccc2C#N)c(C)c1[N+](=O)[O-]]

In analogy to intermediate 1B), 1.49 g (10.6 mmol) 3,5-dimethyl-4-nitro-1H-pyrazole and 2.50 g (12.7 mmol) 2-(bromomethyl)nicotinonitrile were reacted to give after purification of the crude product via a Biotage chromatography system (50 g snap KP-Sil column, hexane/0-100% ethyl acetate, then ethyl acetate/0-15% methanol) 2.16 g (75%) of the desired title compound.

¹H-NMR (300 MHz, DMSO d₆) δ (ppm)=2.36 (s, 3H), 2.63 (s, 3H), 5.69 (s, 2H), 7.57 (dd, 1H), 8.38 (dd, 1H), 8.75 (dd, 1H).

### Intermediate 41B

### 4-[(3,5-dimethyl-4-nitro-1H-pyrazol-1-yl)methyl]-N-(2-hydroxyethyl)benzamide

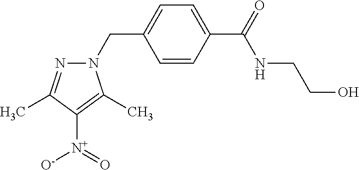 [SMILES: Cc1nn(Cc2ccc(C(=O)NCCO)cc2)c(C)c1[N+](=O)[O-]]

### Step 1: 4-[(3,5-dimethyl-4-nitro-1H-pyrazol-1-yl)methyl]benzoic acid

To a solution of 1.0 g (3.468 mmol) methyl 4-[(3,5-dimethyl-4-nitro-1H-pyrazol-1-yl)methyl]benzoate (intermediate 31B) in 10 mL methanol and 1 mL THF an aq. solution of 2.56 g (63.9 mmol) sodium hydroxide in 20 mL water was added. This mixture was heated at 40° C. for 3 hours and evaporated after cooling to 25° C. To the residue was added 10 mL water and then aq. 10% sulfuric acid up to pH 3. The resulting solid was isolated by filtration and dried yielding 890 mg (91%) of 4-[(3,5-dimethyl-4-nitro-1H-pyrazol-1-yl)methyl]benzoic acid.

¹H-NMR (400 MHz, DMSO d₆) δ (ppm)=2.40 (s, 3H), 2.56 (s, 3H), 5.45 (s, 2H), 7.28 (d, 2H), 7.91 (d, 2H), 12.83 (br. s., 1H).

### Step 2: 4-[(3,5-dimethyl-4-nitro-1H-pyrazol-1-yl)methyl]-N-(2-hydroxyethyl)benzamide

To a solution of 890 mg (3.23 mmol) of the acid prepared in step 1) of intermediate 41B) in 10 mL DMSO was added 1.84 g (4.85 mmol) HATU, 0.85 mL N,N-diisopropylethylamine and 0.23 mL (3.88 mmol) 2-aminoethanol. The reaction mixture was stirred for 2 hours at 25° C. To this mixture was added water and then it was extracted two times with 30 mL ethyl acetate. The combined organic phases were washed with brine, dried over sodium sulfate, filtered and evaporated. The crude product was purified via a Biotage chromatography system (25 g snap KP-Sil column, hexane/0-100% ethyl acetate, then ethyl acetate/0-50% methanol) yielding 1.1 g (107%) of the desired, not completely pure compound.

¹H-NMR (400 MHz, DMSO d₆) δ (ppm)=2.40 (s, 3H), 2.57 (s, 3H), 3.30 (q, 2H), 3.48 (q, 2H), 4.68 (t, 1H), 5.41 (s, 2H), 7.25 (d, 2H), 7.81 (d, 2H), 8.39 (t, 1H).

### Intermediate 42B

### 1-(3-fluoro-4-methoxybenzyl)-3,5-dimethyl-4-nitro-1H-pyrazole

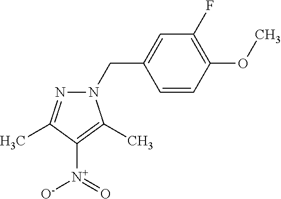

In analogy to intermediate 1B), 1.07 g (7.61 mmol) 3,5-dimethyl-4-nitro-1H-pyrazole and 2.00 g (9.13 mmol) 3-fluoro-4-methoxybenzylbromide were reacted to give after purification of the crude product via a Biotage chromatography system (25 g snap KP-Sil column, hexane/10-100% ethyl acetate, then ethyl acetate/0-25% methanol) 2.09 g (93%) of the desired title compound.

¹H-NMR (500 MHz, DMSO d₆) δ (ppm)=2.39 (s, 3H), 2.58 (s, 3H), 3.80 (s, 3H), 5.27 (s, 2H), 6.98-7.02 (m, 1H), 7.07-7.15 (m, 2H).

### Intermediate 43B

### 2-methoxy-5-{[5-methyl-4-nitro-3-(trifluoromethyl)-1H-pyrazol-1-yl]methyl}pyridine

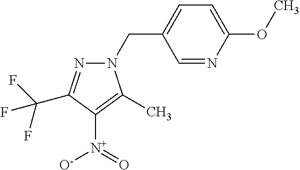 [SMILES: COc1ccc(Cn2nc(C(F)(F)F)c([N+](=O)[O-])c2C)cn1]

In analogy to intermediate 1B), 547 mg (2.80 mmol) 5-methyl-4-nitro-3-(trifluoromethyl)-1H-pyrazole and 530 mg (3.36 mmol) 5-(chloromethyl)-2-methoxypyridine (Journal of Organic Chemistry, 2011, 8336) were reacted to give after purification of the crude product via a Biotage chromatography system (25 g snap KP-Sil column, hexane/10-100% ethyl acetate, then ethyl acetate/0-25% methanol) 800 mg (81%) of the desired title compound.

¹H-NMR (400 MHz, DMSO d₆) δ (ppm)=2.69 (s, 3H), 3.83 (s, 3H), 5.48 (s, 2H), 6.82 (d, 1H), 7.63 (dd, 1H), 8.18 (d, 1H).

### Intermediate 44B

### 1-[3-(4-methoxyphenyl)propyl]-3,5-dimethyl-4-nitro-1H-pyrazole

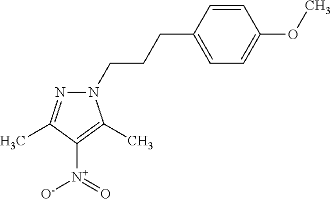

In analogy to intermediate 1B), 2.00 g (14.2 mmol) 3,5-dimethyl-4-nitro-1H-pyrazole and 3.14 g (17.0 mmol) 1-(3-chloropropyl)-4-methoxybenzene (commercially available at Ablock Pharmatech Inc. or Matrix Scientific) were reacted to give after purification of the crude product via a Biotage chromatography system (50 g snap KP-Sil column, hexane/0-70% ethyl acetate) 2.91 g (67%) of the desired title compound.

¹H-NMR (400 MHz, DMSO d₆) δ (ppm)=1.94-2.04 (m, 2H), 2.38 (s, 3H), 2.50-2.55 (m, 5H), 3.70 (s, 3H), 4.04 (t, 2H), 6.81-6.85 (m, 2H), 7.08-7.13 (m, 2H).

### Intermediate 45B

### 4-[(3,5-dimethyl-4-nitro-1H-pyrazol-1-yl)methyl]nicotinonitrile

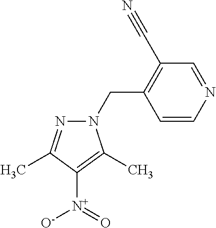 [SMILES: Cc1nn(Cc2ccncc2C#N)c(C)c1[N+](=O)[O-]]

In analogy to intermediate 1B), 1.06 g (7.50 mmol) 3,5-dimethyl-4-nitro-1H-pyrazole and 2.50 g (8.99 mmol) 4-(Bromomethyl)nicotinonitrile Hydrobromid (commercially available at Santai Labs; parent compound commercially available at e.g. Aquila Pharmatech or Ellanova Laboratories) were reacted to give after two subsequent purifications of the crude product via a Biotage chromatography system (25 g and secondly 50 g snap KP-Sil column, hexane/0-100% ethyl acetate, then ethyl acetate/0-90% methanol) 1.46 g (72%) of the desired title compound.

¹H-NMR (400 MHz, DMSO d₆) δ (ppm)=2.39 (s, 3H), 2.62 (s, 3H), 5.62 (s, 2H), 7.15 (d, 1H), 8.78 (d, 1H), 9.04 (s, 1H).

### Intermediate 46B

### 3,5-dimethyl-4-nitro-1-(2-phenoxyethyl)-1H-pyrazole

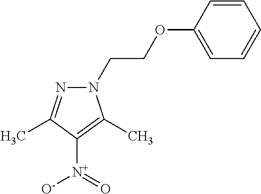 [SMILES: Cc1nn(CCOc2ccccc2)c(C)c1[N+](=O)[O-]]

In analogy to intermediate 1B), 780 mg (5.53 mmol) 3,5-dimethyl-4-nitro-1H-pyrazole and 1.50 g (6.64 mmol) 4-(2-bromoethoxy)benzonitrile ([CAS-No. 37142-39-5], commercially available at e.g. Combi-Blocks Inc. or ACC Corporation) were reacted to give after purification of the crude product via a Biotage chromatography system (50 g snap KP-Sil column, hexane/0-70% ethyl acetate) 1.41 g (86%) of the desired title compound.

¹H-NMR (400 MHz, DMSO d₆) δ (ppm)=2.39 (s, 3H), 2.64 (s, 3H), 4.43 (t, 2H), 4.52 (t, 2H), 7.07-7.14 (m, 2H), 7.73-7.80 (m, 2H).

### Intermediate 47B

### 4-[(3,5-dimethyl-4-nitro-1H-pyrazol-1-yl)methyl]-3-fluorobenzonitrile

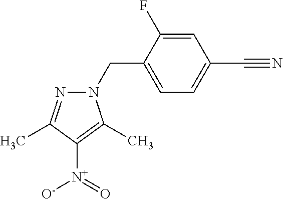 [SMILES: Cc1nn(Cc2ccc(C#N)cc2F)c(C)c1[N+](=O)[O-]]

In analogy to intermediate 1B), 934 mg (6.62 mmol) 3,5-dimethyl-4-nitro-1H-pyrazole and 1.70 g (7.94 mmol) 4-(bromomethyl)-3-fluorobenzonitrile ([CAS-No. 105942-09-4], commercially available at e.g. ABCR) were reacted to give after purification of the crude product via a Biotage chromatography system (50 g snap KP-Sil column, hexane/0-100% ethyl acetate) 1.84 g (91%) of the desired title compound.

¹H-NMR (400 MHz, DMSO d₆) δ (ppm)=2.38 (s, 3H), 2.63 (s, 3H), 5.50 (s, 2H), 7.31 (t, 1H), 7.70 (dd, 1H), 7.92 (dd, 1H).

### Intermediate 48B

### 2-{[5-methyl-4-nitro-3-(trifluoromethyl)-1H-pyrazol-1-yl]methyl}benzonitrile

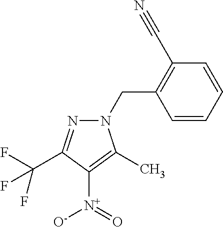 [SMILES: Cc1c([N+](=O)[O-])c(C(F)(F)F)nn1Cc1ccccc1C#N]

2.07 g (10.6 mmol) 5-methyl-4-nitro-3-(trifluoromethyl)-1H-pyrazole together with 2.50 (12.8 mmol) 2-(bromomethyl)benzonitrile were dissolved in 37 mL DMSO. After the addition of 2.4 ml (15.9 mmol) DBU the reaction mixture was stirred at RT for 18 hours. The mixture was then diluted with 150 ml ethyl acetate and the organic phase was washed with water, half-concentrated brine, dried over sodium sulfate, filtered and evaporated to obtain a crude product. This material was purified via a Biotage chromatography system (25 g snap KP-Sil column, hexane/0-100% ethyl acetate) to yield 2.5 g (70%) of the desired title compound.

¹H-NMR (400 MHz, DMSO d₆) δ (ppm)=2.72 (s, 3H), 5.75 (s, 2H), 7.32 (d, 1H), 7.55-7.61 (m, 1H), 7.73 (td, 1H), 7.94 (dd, 1H).

### Intermediate 49B

### 1-(4-methoxybenzyl)-5-methyl-4-nitro-3-(trifluoromethyl)-1H-pyrazole

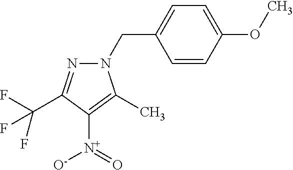 [SMILES: COc1ccc(Cn2nc(C(F)(F)F)c([N+](=O)[O-])c2C)cc1]

In analogy to intermediate 1B), 5.0 g (25.6 mmol) 5-methyl-4-nitro-3-(trifluoromethyl)-1H-pyrazole and 4.82 g (30.8 mmol) 1-(chloromethyl)-4-methoxybenzene were reacted to give after purification of the crude product via a Biotage chromatography system (50 g snap KP-Sil column, hexane/0-100% ethyl acetate) 6.7 g (79%) of the desired title compound.

¹H-NMR (300 MHz, DMSO d₆) δ (ppm)=2.65 (s, 3H), 3.73 (s, 3H), 5.45 (s, 2H), 6.93 (d, 2H), 7.22 (d, 2H).

### Intermediate 50B

### 5-[(3,5-dimethyl-4-nitro-1H-pyrazol-1-yl)methyl]-2-methoxypyridine

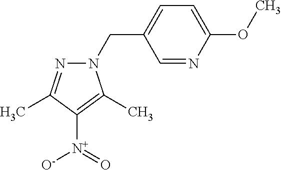

In analogy to intermediate 1B), in two experiments 187 mg (1.32 mmol)/2.31 g (16.4 mmol) 3,5-dimethyl-4-nitro-1H-pyrazole and 250 mg (1.59 mmol)/3.10 g (19.7 mmol) 5-(chloromethyl)-2-methoxypyridine (Journal of Organic Chemistry, 2011, 8336) were reacted to give after two subsequent purifications of the combined crude products via a Biotage chromatography system (50 g snap KP-Sil column, hexane/10-100% ethyl acetate, then ethyl acetate/0-25% methanol and secondly 50 g snap KP-Sil column, hexane/10-100% ethyl acetate) 3.23 g (70%) of the desired title compound.

¹H-NMR (300 MHz, DMSO d₆) δ (ppm)=2.38 (s, 3H), 2.62 (s, 3H), 3.82 (s, 3H), 5.29 (s, 2H), 6.79 (d, 1H), 7.58 (dd, 1H), 8.13 (d, 1H).

### Intermediate 51B

### 4-{[5-methyl-4-nitro-3-(trifluoromethyl)-1H-pyrazol-1-yl]methyl}nicotinonitrile

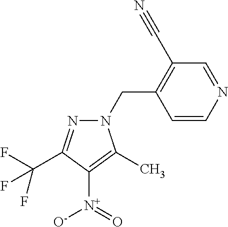 [SMILES: Cc1c([N+](=O)[O-])c(C(F)(F)F)nn1Cc1ccncc1C#N]

In analogy to intermediate 1B), in two experiment 250 mg (1.28 mmol)/1.17 g (6.0 mmol) 5-methyl-4-nitro-3-(trifluoromethyl)-1H-pyrazole and 427 mg (1.54 mmol)/2.0 g (7.20 mmol) 4-(Bromomethyl)nicotinonitrile Hydrobromid (commercially available at Santai Labs; parent compound commercially available at e.g. Aquila Pharmatech or Ellanova Laboratories) were reacted to give after two subsequent purifications of the combined crude products via a Biotage chromatography system (50 g snap KP-Sil column, hexane/10-100% ethyl acetate, then ethyl acetate/0-45% methanol and secondly 25 g snap KP-Sil column, hexane/10-100% ethyl acetate, then ethyl acetate/0-5% methanol) 1.2 g (50%) of the desired title compound.

¹H-NMR (400 MHz, DMSO d₆) δ (ppm)=2.67 (s, 3H), 5.83 (s, 2H), 7.24 (dd, 1H), 8.81 (d, 1H), 9.07 (s, 1H).

### Intermediate 52B

### 5-{[5-methyl-4-nitro-3-(trifluoromethyl)-1H-pyrazol-1-yl]methyl}pyridine-2-carbonitrile

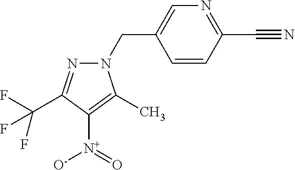 [SMILES: Cc1c([N+](=O)[O-])c(C(F)(F)F)nn1Cc1ccc(C#N)nc1]

In analogy to intermediate 1B), 2.48 g (12.7 mmol) 5-methyl-4-nitro-3-(trifluoromethyl)-1H-pyrazole and 3.00 g (15.2 mmol) 5-(bromomethyl)pyridine-2-carbonitrile ([308846-06-2], commercially available e.g. Fluorochem, Apollo Scientific) were reacted to give after two subsequent purification of the crude product via a Biotage chromatography system (50 g snap KP-Sil column, hexane/0-100% ethyl acetate, then ethyl acetate/0-25% methanol)) 2.46 g (60%) of the desired title compound.

¹H-NMR (400 MHz, DMSO d₆) δ (ppm)=2.68 (s, 3H), 5.73 (s, 2H), 7.90 (dd, 1H), 8.07 (d, 1H), 8.73 (d, 1H).

### Intermediate 53B

### 3-fluoro-4-{[5-methyl-4-nitro-3-(trifluoromethyl)-1H-pyrazol-1-yl]methyl}benzonitrile

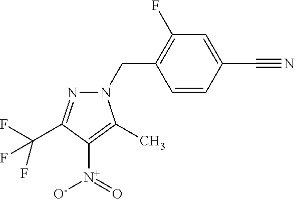 [SMILES: Cc1c([N+](=O)[O-])c(C(F)(F)F)nn1Cc1ccc(C#N)cc1F]

In analogy to intermediate 1B), 1.29 g (6.62 mmol) 5-methyl-4-nitro-3-(trifluoromethyl)-1H-pyrazole and 1.70 g (7.94 mmol) 4-(bromomethyl)-3-fluorobenzonitrile ([CAS-No. 105942-09-4], commercially available at e.g. ABCR) were reacted to give after purification of the crude product via a Biotage chromatography system (50 g snap KP-Sil column, hexane/0-100% ethyl) 1.89 g (78%) of the desired title compound.

¹H-NMR (400 MHz, DMSO d₆) δ (ppm)=2.68 (s, 3H), 5.70 (s, 2H), 7.43 (t, 1H), 7.73 (dd, 1H), 7.95 (dd, 1H).

### Intermediate 54B

### 1-(4-fluorobenzyl)-5-methyl-4-nitro-1H-pyrazole-3-carbonitrile

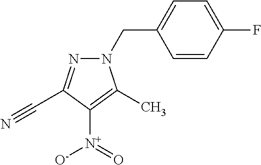 [SMILES: Cc1c([N+](=O)[O-])c(C#N)nn1Cc1ccc(F)cc1]

In analogy to intermediate 1B), 2.008 g (13.1 mmol) 5-methyl-4-nitro-1H-pyrazole-3-carbonitrile (prepared according to Journal of Heterocyclic Chemistry, 1970, p. 863; or its tautomer) and 2.98 g (15.8 mmol) 1-(bromomethyl)-4-fluorobenzene were reacted to give after three subsequent purifications of the crude product via a Biotage chromatography system (two times 50 g snap KP-Sil column, hexane/0-100% ethyl acetate, then ethyl acetate/0-10% methanol and finally 50 g snap KP-Sil column, hexane/0-100% ethyl acetate) 2.66 g (78%, purity about 90%) of the desired title compound.

¹H-NMR (400 MHz, DMSO d₆) δ (ppm)=2.64 (s, 3H), 5.54 (s, 2H), 7.16-7.24 (m, 4H).

### Intermediate 55B

### 4-[(3,5-diethyl-4-nitro-1H-pyrazol-1-yl)methyl]benzonitrile

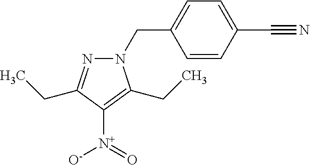 [SMILES: CCc1nn(Cc2ccc(C#N)cc2)c(CC)c1[N+](=O)[O-]]

In analogy to intermediate 48B), 800 mg (4.73 mmol) 3,5-diethyl-4-nitro-1H-pyrazole (intermediate 1D) and 1.11 g (5.67 mmol) 4-(bromomethyl)benzonitrile were reacted to give after purification of the crude product via a Biotage chromatography system (25 g snap KP-Sil column, hexane/0-99% ethyl acetate) 1.30 g (94%) of the desired title compound.

¹H-NMR (400 MHz, DMSO d₆) δ (ppm)=1.00 (t, 3H), 1.18 (t, 3H), 2.86 (q, 2H), 2.98 (d, 2H), 5.52 (s, 2H), 7.35 (d, 2H), 7.83 (d, 2H).

### Intermediate 56B

### 4-{[5-ethyl-4-nitro-3-(trifluoromethyl)-1H-pyrazol-1-yl]methyl}benzonitrile

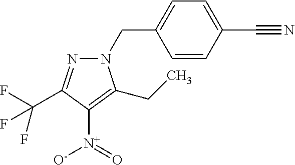 [SMILES: CCc1c([N+](=O)[O-])c(C(F)(F)F)nn1Cc1ccc(C#N)cc1]

In analogy to intermediate 48B), 1.00 g (4.78 mmol) 5-ethyl-4-nitro-3-(trifluoromethyl)-1H-pyrazole (intermediate 2D) and 1.13 g (5.74 mmol) 4-(bromomethyl)benzonitrile were reacted to give after purification of the crude product via a Biotage chromatography system (25 g snap KP-Sil column, hexane/10-90% ethyl acetate) 1.21 g (72%) of the desired title compound.

¹H-NMR (400 MHz, DMSO d₆) δ (ppm)=1.06 (t, 3H), 3.07 (q, 2H), 5.71 (s, 2H), 7.43 (d, 2H), 7.88 (d, 2H).

### Intermediate 57B and 58B

### 4-{[5-isopropyl-4-nitro-3-(trifluoromethyl)-1H-pyrazol-1-yl]methyl}benzonitrile and 4-{[3-isopropyl-4-nitro-5-(trifluoromethyl)-1H-pyrazol-1-yl]methyl}benzonitrile

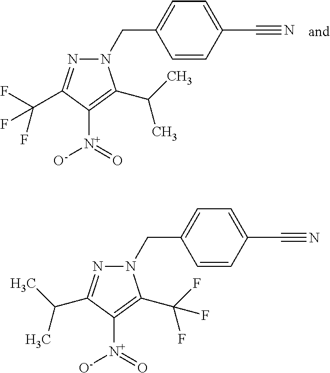 [SMILES: CC(C)c1c([N+](=O)[O-])c(C(F)(F)F)nn1Cc1ccc(C#N)cc1 ; CC(C)c1nn(Cc2ccc(C#N)cc2)c(C(F)(F)F)c1[N+](=O)[O-]]

In analogy to intermediate 48B), 1.50 g (6.72 mmol) 5-isopropyl-4-nitro-3-(trifluoromethyl)-1H-pyrazole (intermediate 3D) and 1.58 g (8.07 mmol) 4-(bromomethyl)benzonitrile were reacted to give after purification of the crude product mixture via two subsequent Biotage chromatography system (25 g snap KP-Sil column, hexane/10-90% ethyl acetate and finally 25 g snap KP-Sil column, hexane/10-60% ethyl acetate) 1.26 g (50%) of 4-{[5-isopropyl-4-nitro-3-(trifluoromethyl)-1H-pyrazol-1-yl]methyl}benzonitrile and 400 mg (17%) of the regioisomer 4-{[3-isopropyl-4-nitro-5-(trifluoromethyl)-1H-pyrazol-1-yl]methyl}benzonitrile as the desired compounds.

### 4-{[5-isopropyl-4-nitro-3-(trifluoromethyl)-1H-pyrazol-1-yl]methyl}benzonitrile (intermediate 57B)

¹H-NMR (400 MHz, DMSO d₆) δ (ppm)=1.23 (d, 5H), 3.58 (spt, 1H), 5.77 (s, 2H), 7.38 (d, 2H), 7.86 (d, 2H).

### 4-{[3-isopropyl-4-nitro-5-(trifluoromethyl)-1H-pyrazol-1-yl]methyl}benzonitrile (intermediate 58B)

¹H-NMR (400 MHz, DMSO d₆) δ (ppm)=1.29 (d, 6H), 3.47 (spt, 1H), 5.77 (s, 2H), 7.32 (d, 2H), 7.87 (d, 2H).

### Intermediate 59B and 60B

### 4-[(3-isopropyl-5-methyl-4-nitro-1H-pyrazol-1-yl)methyl]benzonitrile and 4-[(5-isopropyl-3-methyl-4-nitro-1H-pyrazol-1-yl)methyl]benzonitrile

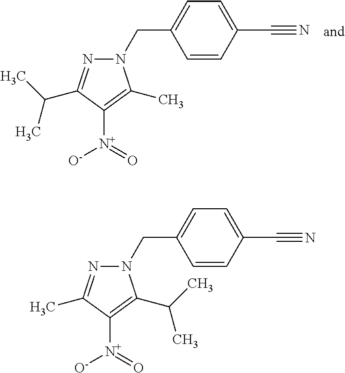 [SMILES: Cc1c([N+](=O)[O-])c(C(C)C)nn1Cc1ccc(C#N)cc1 ; Cc1nn(Cc2ccc(C#N)cc2)c(C(C)C)c1[N+](=O)[O-]]

In analogy to intermediate 48B), 2.00 g (11.8 mmol) 3-isopropyl-5-methyl-4-nitro-1H-pyrazole (intermediate 4D) and 2.78 g (14.2 mmol) 4-(bromomethyl)benzonitrile were reacted to give after purification of the crude product mixture via a Biotage chromatography system (100 g snap KP-Sil column, hexane/0-100% ethyl acetate) 3.22 g (96%) of 4-[(3-isopropyl-5-methyl-4-nitro-1H-pyrazol-1-yl)methyl]benzonitrile and its regioisomer 4-[(5-isopropyl-3-methyl-4-nitro-1H-pyrazol-1-yl)methyl]benzonitrile as a mixture of desired compounds.

NMR of 4-[(3-isopropyl-5-methyl-4-nitro-1H-pyrazol-1-yl)methyl]benzonitrile as the main product:

¹H-NMR (300 MHz, DMSO d₆) δ (ppm)=1.21 (d, 6H), 2.53 (s, 3H), 3.47 (spt, 1H), 5.52 (s, 2H), 7.30 (d, 2H), 7.83 (d, 2H).

### Intermediate 61B and 62B

### 4-[(3-ethyl-5-methyl-4-nitro-1H-pyrazol-1-yl)methyl]benzonitrile and 4-[(5-ethyl-3-methyl-4-nitro-1H-pyrazol-1-yl)methyl]benzonitrile

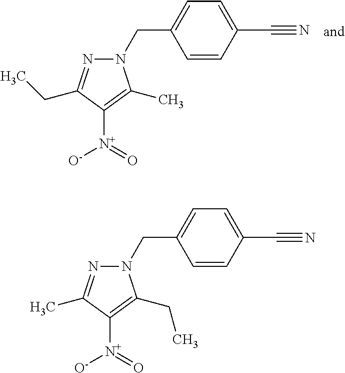 [SMILES: CCc1nn(Cc2ccc(C#N)cc2)c(C)c1[N+](=O)[O-] ; CCc1c([N+](=O)[O-])c(C)nn1Cc1ccc(C#N)cc1]

In analogy to intermediate 48B), 490 mg (3.16 mmol) 3-ethyl-5-methyl-4-nitro-1H-pyrazole (intermediate 5D) and 743 mg (3.79 mmol) 4-(bromomethyl)benzonitrile were reacted to give after purification of the crude product mixture via a Biotage chromatography system (25 g snap KP-Sil column, hexane/10-90% ethyl acetate) 656 mg (77%) of 4-[(3-ethyl-5-methyl-4-nitro-1H-pyrazol-1-yl)methyl]benzonitrile and its regioisomer 4-[(5-ethyl-3-methyl-4-nitro-1H-pyrazol-1-yl)methyl]benzonitrile as a mixture of desired compounds.

NMR of the mixture:

¹H-NMR (400 MHz, DMSO d₆) δ (ppm)=1.00/1.17 (t, 3H), 2.41/2.55 (s, 3H), 2.84/2.99 (q, 2H), 5.50 (s, 2H), 7.31-7.38 (m, 2H), 7.81-7.86 (m, 2H).

### Intermediate 63B

### (±)-4-[1-(3,5-dimethyl-4-nitro-1H-pyrazol-1-yl)ethyl]benzonitrile

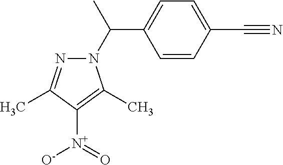 [SMILES: Cc1nn(C(C)c2ccc(C#N)cc2)c(C)c1[N+](=O)[O-]]

In analogy to intermediate 48B), 823 mg (5.83 mmol) 3,5-dimethyl-4-nitro-1H-pyrazole and 1.47 g (7.00 mmol) 4-(1-bromoethyl)benzonitrile (commercially available e.g. Bio Farma Ltd. or Enamine or prepared according to WO2012/11707 A2, Page/Page column 29-30) were reacted to give after purification of the crude product mixture via a Biotage chromatography system (25 g snap KP-Sil column, hexane/10-90% ethyl acetate) 449 mg (27%) of the desired compound.

¹H-NMR (300 MHz, DMSO d₆) δ (ppm)=1.78 (d, 3H), 2.42 (s, 3H), 2.55 (s, 3H), 5.88 (q, 1H), 7.43 (d, 2H), 7.79-7.86 (m, 2H).

### Intermediate 64B

### (±)-4-{1-[5-methyl-4-nitro-3-(trifluoromethyl)-1H-pyrazol-1-yl]ethyl}benzonitrile

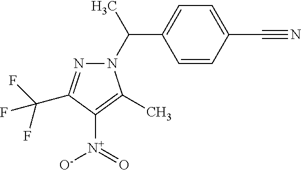 [SMILES: Cc1c([N+](=O)[O-])c(C(F)(F)F)nn1C(C)c1ccc(C#N)cc1]

In analogy to intermediate 48B), 1.54 g (7.89 mmol) 5-methyl-4-nitro-3-(trifluoromethyl)-1H-pyrazole and 1.99 g (9.47 mmol) 4-(1-bromoethyl)benzonitrile (commercially available e.g. Bio Farma Ltd. or Enamine or prepared according to WO2012/11707 A2, Page/Page column 29-30) were reacted to give after purification of the crude product mixture via a Biotage chromatography system (25 g snap KP-Sil column, hexane/10-90% ethyl acetate) 1.56 g (55%) of the desired compound.

¹H-NMR (400 MHz, DMSO d₆) δ (ppm)=1.81 (d, 3H), 2.61 (s, 3H), 6.07 (q, 1H), 7.43-7.49 (m, 2H), 7.82-7.89 (m, 2H).

### Intermediate 65B

### (±)-5-methyl-4-nitro-1-(1-phenylethyl)-3-(trifluoromethyl)-1H-pyrazole

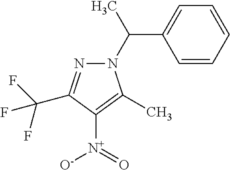 [SMILES: Cc1c([N+](=O)[O-])c(C(F)(F)F)nn1C(C)c1ccccc1]

In analogy to intermediate 48B), 1.00 g (5.13 mmol) 5-methyl-4-nitro-3-(trifluoromethyl)-1H-pyrazole and 1.04 g (5.64 mmol) (1-bromoethyl)benzene were reacted to give after purification of the crude product mixture via a Biotage chromatography system (50 g snap KP-Sil column, hexane/0-100% ethyl acetate) 1.03 g (64%) of the desired compound.

¹H-NMR (400 MHz, DMSO d₆) δ (ppm)=1.81 (d, 3H), 2.61 (s, 3H), 5.94 (q, 1H), 7.24-7.41 (m, 5H).

### Intermediate 66B

### 4-[3-(3,5-dimethyl-4-nitro-1H-pyrazol-1-yl)propyl]benzonitrile

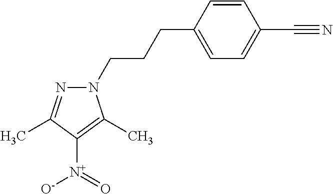 [SMILES: Cc1nn(CCCc2ccc(C#N)cc2)c(C)c1[N+](=O)[O-]]

In analogy to intermediate 48B), 750 mg (5.31 mmol) 3,5-dimethyl-4-nitro-1H-pyrazole and 1.43 g (6.38 mmol) 4-(3-bromopropyl)benzonitrile (prepared according to Pharmazie, 1982, p. 178 or WO2005/123747, 2005; Page/Page column 53-54) were reacted to give after purification of the crude product mixture via a Biotage chromatography system (25 g snap KP-Sil column, hexane/30-100% ethyl acetate) 1.06 g (68%) of the desired compound.

¹H-NMR (300 MHz, DMSO d₆) δ (ppm)=2.05 (quin, 2H), 2.37 (s, 3H), 2.53 (s, 3H), 2.68 (t, 2H), 4.08 (t, 2H), 7.42 (d, 2H), 7.73 (d, 2H).

### Intermediate 67B

### 6-[(3,5-dimethyl-4-nitro-1H-pyrazol-1-yl)methyl]nicotinonitrile

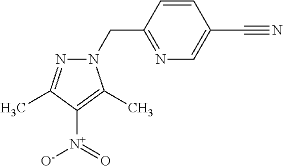 [SMILES: Cc1nn(Cc2ccc(C#N)cn2)c(C)c1[N+](=O)[O-]]

In analogy to intermediate 1B), 1.19 g (8.46 mmol) 3,5-dimethyl-4-nitro-1H-pyrazole and 2.00 g (10.2 mmol) 6-(bromomethyl)nicotinonitrile ([CAS-No. 158626-15-4], commercially available e.g. Fluorchem, BePharm, FCH Group Company) were reacted to give after purification of the crude product via a Biotage chromatography system (25 g snap KP-Sil column, hexane/0-100% ethyl acetate, then ethyl acetate/0-15% methanol) 2.11 g (87%) of the desired compound.

¹H-NMR (400 MHz, DMSO d₆) δ (ppm)=2.37 (s, 3H), 2.58 (s, 3H), 5.59 (s, 2H), 7.48 (d, 1H), 8.32 (dd, 1H), 8.95 (dd, 1H).

### Intermediate 68B

### 6-{[5-methyl-4-nitro-3-(trifluoromethyl)-1H-pyrazol-1-yl]methyl}nicotinonitrile

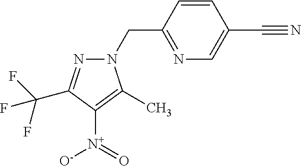 [SMILES: Cc1c([N+](=O)[O-])c(C(F)(F)F)nn1Cc1ccc(C#N)cn1]

In analogy to intermediate 1B), 1.65 g (8.46 mmol) 5-methyl-4-nitro-3-(trifluoromethyl)-1H-pyrazole and 2.00 g (10.2 mmol) 6-(bromomethyl)nicotinonitrile ([CAS-No. 158626-15-4], commercially available e.g. Fluorchem, BePharm, FCH Group Company) were reacted to give after purification of the crude product via a Biotage chromatography system (25 g snap KP-Sil column, hexane/0-100% ethyl acetate, then ethyl acetate/0-15% methanol) 2.51 g (86%) of the desired compound.

¹H-NMR (400 MHz, DMSO d₆) δ (ppm)=2.63 (s, 3H), 5.82 (s, 2H), 7.62 (dd, 1H), 8.36 (dd, 1H), 8.96 (dd, 1H).

### Intermediate 69B

### methyl 4-{[5-methyl-4-nitro-3-(trifluoromethyl)-1H-pyrazol-1-yl]methyl}benzoate

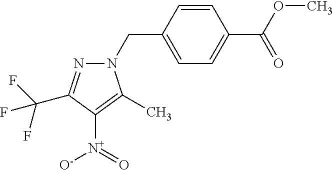 [SMILES: COC(=O)c1ccc(Cn2nc(C(F)(F)F)c([N+](=O)[O-])c2C)cc1]

In analogy to intermediate 1B), 2.50 g (12.8 mmol) 5-methyl-4-nitro-3-(trifluoromethyl)-1H-pyrazole and 3.52 g (15.4 mmol) methyl 4-(bromomethyl)benzoate were reacted to give after purification of the crude product via a Biotage chromatography system (50 g snap KP-Sil column, hexane/20-80% ethyl acetate) 3.86 g (85%) of the desired compound.

¹H-NMR (400 MHz, DMSO d₆) δ (ppm)=2.63 (s, 3H), 3.84 (s, 3H), 5.65 (s, 2H), 7.36 (d, 2H), 7.92-7.98 (m, 2H).

### Intermediate 70B

### (±)-ethyl [{4-[(3,5-dimethyl-4-nitro-1H-pyrazol-1-yl)methyl]phenyl}(methyl)oxido-λ⁶-sulfanylidene]carbamate

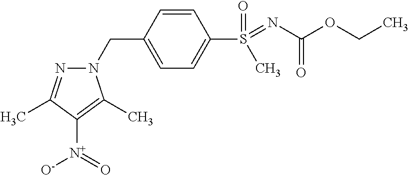 [SMILES: CCOC(=O)N=S(C)(=O)c1ccc(Cn2nc(C)c([N+](=O)[O-])c2C)cc1]

### Step 1:

In analogy to intermediate 1B), 3.30 g (23.4 mmol) 3,5-dimethyl-4-nitro-1H-pyrazole and 4.85 g (28.1 mmol) 1-(chloromethyl)-4-(methylsulfanyl)benzene were reacted to give after purification of the crude product via a Biotage chromatography system (100 g snap KP-Sil column, hexane/0-100% ethyl acetate, then ethyl acetate/0-10% methanol) 5.47 g (76%) 3,5-dimethyl-1-[4-(methylsulfanyl)benzyl]-4-nitro-1H-pyrazole.

¹H-NMR (300 MHz, DMSO d₆) δ (ppm)=2.39 (s, 3H), 2.44 (s, 3H), 2.57 (s, 3H), 5.30 (s, 2H), 7.10-7.18 (m, 2H), 7.20-7.26 (m, 2H).

### Step 2

To a stirred solution of 550 mg (1.98 mmol) of 3,5-dimethyl-1-[4-(methylsulfanyl)benzyl]-4-nitro-1H-pyrazole from step 1 intermediate 70B in 6.38 mL dichloromethane was given portionwise 323 mg (1.87 mmol) meta-chloroperoxybenzoic acid (MCPBA) at a temperature of 10-20° C. Then the mixture was stirred for an additional 30 minutes at room temperature. After diluting the mixture with 50 mL dichloromethane this organic phase was washed with 30 mL saturated aq. NaHSO3-solution, brine, dried over sodium sulfate, filtered and evaporated to dryness. This raw material together with the raw material of a analogous second experiment using 4.95 g (17.8 mmol) of 3,5-dimethyl-1-[4-(methylsulfanyl)benzyl]-4-nitro-1H-pyrazole from step 1 intermediate 70B product was purified via a Biotage chromatography system (100 g snap KP-Sil column, hexane/0-100% ethyl acetate, then ethyl acetate/0-80% methanol) to obtain 3.47 g (60%) (±)-3,5-dimethyl-1-[4-(methylsulfinyl)benzyl]-4-nitro-1H-pyrazole.

¹H-NMR (400 MHz, DMSO d₆) δ (ppm)=2.42 (s, 3H), 2.61 (s, 3H), 2.74 (s, 3H), 5.46 (s, 2H), 7.41 (d, 2H), 7.66-7.70 (m, 2H).

### Step 3

To a stirred solution of 3.48 g (11.9 mmol) of (±)-3,5-dimethyl-1-[4-(methylsulfinyl)benzyl]-4-nitro-1H-pyrazole from step 2 intermediate 70B in 5.00 mL dichloromethane was given 1.54 g (23.7 mmol) sodium azide and then carefully at 0° C. 3.16 mL conc. sulfuric acid (gas ecolution). After stirring at 25° C. for 1 hour the mixture was heated up to 40° C. and stirred at this temperature for about 1 day. After cooling to 0° C. additional 0.75 g (11.8 mmol) sodium azide was added and stirring at 40° C. was continued for 3 days. While cooling at 0° C. 60 mL of ice water was carefully added to the reaction mixture. The reaction mixture was extracted three times with dichloromethane. This first combined organic phases were washed with 25% aq. sodium hydroxide solution, dried over sodium sulfate, filtered and evaporated to dryness.

White cooling at 0° C. the pH of the aqueous phase from the first extraction was adjusted up to 9 with aq. 25% sodium hydroxide solution. Then this aqueous phase was extracted three times with dichloromethane. The combined organic phases were dried over sodium sulfate, filtered and evaporated to dryness.

The combined crude products were purified via a Biotage chromatography system (50 g snap KP-Sil column, hexane/0-100% ethyl acetate, then ethyl acetate/0-20% methanol) to obtain 2.44 g of a mixture of the starting material (±)-3,5-dimethyl-1-[4-(methylsulfinyl)benzyl]-4-nitro-1H-pyrazole and the product (±)-3,5-dimethyl-1-[4-(methylsulfonimidoyl)benzyl]-4-nitro-1H-pyrazole in a ratio of 1:2.

To a solution of 2.44 g of this mixture in 64.0 mL pyridine was added 3.78 mL (39.6 mmol) ethyl chloroformate at room temperature and stirred at this tempareture for 16 hours. The reaction mixture was poured into brine and then extracted three times with ethyl acetate. The combined organic phases were evaporated to dryness then toluene was added and the mixture was evaporated to dryness again. The toluene addition and evaporation process was repeated two times more and then the crude product was purified via a Biotage chromatography system (50 g snap KP-Sil column, hexane/0-100% ethyl acetate, then ethyl acetate/0-75% methanol) to obtain 2.15 g (69%) of the desired compound.

¹H-NMR (300 MHz, DMSO d₆) δ (ppm)=1.06 (t, 3H), 2.40 (s, 3H), 2.60 (s, 3H), 3.43 (s, 3H), 3.79-3.97 (m, 2H), 5.51 (s, 2H), 7.46 (d, 2H), 7.91 (d, 2H).

### Intermediate 71B

### 1-{4-[(3,5-dimethyl-4-nitro-1H-pyrazol-1-yl)methyl]phenyl}-N,N-dimethylmethanamine

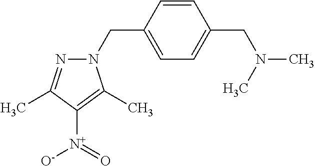

### Step 1:

In analogy to intermediate 1B), 2.67 g (18.9 mmol) 3,5-dimethyl-4-nitro-1H-pyrazole and 15.0 g (56.8 mmol) 1,4-bis(bromomethyl)benzene were reacted to give after four subsequent purifications of the crude product via a Biotage chromatography system (first 100 g, all others 50 g snap KP-Sil column, hexane/10-90% ethyl acetate) 3.02 g (45%) of 1-[4-(bromomethyl)benzyl]-3,5-dimethyl-4-nitro-1H-pyrazole.

¹H-NMR (400 MHz, DMSO d₆) δ (ppm)=2.39 (s, 3H), 2.57 (s, 3H), 4.67 (s, 2H), 5.35 (s, 2H), 7.18 (d, 2H), 7.42 (d, 2H).

### Step 2

To a stirred solution of 100 mg (0.31 mmol) of 1-[4-(bromomethyl)benzyl]-3,5-dimethyl-4-nitro-1H-pyrazole from step 1 intermediate 71B in 2.0 mL DMSO was given 0.19 mL (0.37 mmol) of a 2M solution of dimethylamine in THF followed by 70.4 mg (0.46 mmol) DBU. This mixture was stirred for 20 hours at room temperature. A second experiment was done similar starting with 400 mg (1.23 mmol) 1-[4-(bromomethyl)benzyl]-3,5-dimethyl-4-nitro-1H-pyrazole from step 1 intermediate 71B and 0.74 mL (1.48 mmol) of a 2M solution of dimethylamine in THF. The combined reaction mixtures were diluted with 200 mL ethyl acetate and this resulting organic phase was washed with twice with water, brine, dried over sodium sulfate, filtered and evaporated to dryness. The raw material was purified via a Biotage chromatography system (25 g snap KP-Sil column, ethyl acetate/0-50% methanol) to obtain 340 mg (76%) of the desired title compound.

¹H-NMR (400 MHz, DMSO d₆) δ (ppm)=2.10 (s, 6H), 2.39 (s, 3H), 2.57 (s, 3H), 3.33 (s, 2H), 5.33 (s, 2H), 7.14 (d, 2H), 7.25 (d, 2H).

### Intermediate 72B

### 4-{[3-methyl-4-nitro-5-(trifluoromethyl)-1H-pyrazol-1-yl]methyl}benzonitrile

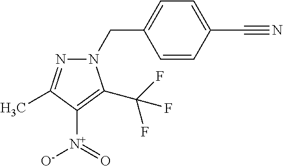 [SMILES: Cc1nn(Cc2ccc(C#N)cc2)c(C(F)(F)F)c1[N+](=O)[O-]]

In analogy to intermediate 1B), 10.0 g (51.3 mmol) 5-methyl-4-nitro-3-(trifluoromethyl)-1H-pyrazole and 12.1 g (61.5 mmol) 4-(bromomethyl)benzonitrile were reacted to give after purification of the crude product via a Biotage chromatography system (100 g snap KP-Sil column, hexane/0-70% ethyl acetate) 4-{[5-methyl-4-nitro-3-(trifluoromethyl)-1H-pyrazol-1-yl]methyl}benzonitrile (intermediate 26B) as the main component and 690 mg (4.3%) of the desired title compound.

¹H-NMR (500 MHz, DMSO d₆) δ (ppm)=2.49 (s, 3H), 5.74 (s, 2H), 7.34 (d, 2H), 7.83-7.87 (m, 2H).

### Intermediate 73B

### 4-[(3,5-dimethyl-4-nitro-1H-pyrazol-1-yl)methyl]-3,5-difluorobenzonitrile

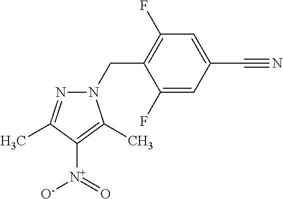 [SMILES: Cc1nn(Cc2c(F)cc(C#N)cc2F)c(C)c1[N+](=O)[O-]]

### Step 1:

To a solution of 1.00 g (6.53 mmol) 3,5-difluoro-4-methylbenzonitrile in 8.0 mL tetrachloromethane was added 1.28 g (7.18 mmol)N-bromosuccinimide and 54 mg (0.33 mmol) azobisisobutyronitrile. The mixture was heated to reflux for 16 hours. After cooling to room temperature this mixture was diluted with ethyl acetate and the resulting organic phase was washed with aq. 1M NaS₂O₃-solution, dried over sodium sulfate, filtered and evaporated to dryness. The resulting raw material was purified via a Biotage chromatography system (25 g snap KP-Sil column, hexane/0-100% ethyl acetate, then ethyl acetate/0-90% methanol) to give 1.6 g (95%) of 4-(bromomethyl)-3,5-difluorobenzonitrile as the desired compound.

¹H-NMR (400 MHz, DMSO d₆) δ (ppm)=4.68 (s, 2H), 7.86-7.93 (m, 2H).

### Step 2:

In analogy to intermediate 1B), 558 mg (3.95 mmol) 3,5-dimethyl-4-nitro-1H-pyrazole and 1.10 g (4.74 mmol) 4-(bromomethyl)-3,5-difluorobenzonitrile from step 1 intermediate 72B were reacted to give after purification of the crude product via a Biotage chromatography system (25 g snap KP-Sil column, hexane/0-100% ethyl acetate, then ethyl acetate/0-80% methanol) 1.03 g (87%) of the desired title compound.

¹H-NMR (400 MHz, DMSO d₆) δ (ppm)=2.31 (s, 3H), 2.68 (s, 3H), 5.42 (s, 2H), 7.83-7.89 (m, 2H).

### Intermediate 74B

### 3,5-difluoro-4-{[5-methyl-4-nitro-3-(trifluoromethyl)-1H-pyrazol-1-yl]methyl}benzonitrile

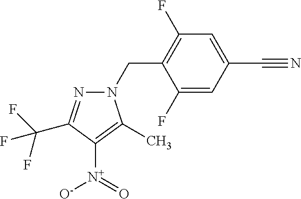 [SMILES: Cc1c([N+](=O)[O-])c(C(F)(F)F)nn1Cc1c(F)cc(C#N)cc1F]

In analogy to intermediate 1B), 1.04 g (5.35 mmol) 5-methyl-4-nitro-3-(trifluoromethyl)-1H-pyrazole and 1.49 g (6.42 mmol) 4-(bromomethyl)-3,5-difluorobenzonitrile from step 1 intermediate 72B were reacted to give after purification of the crude product via a Biotage chromatography system (25 g snap KP-Sil column, hexane/0-100% ethyl acetate, then ethyl acetate/0-80% methanol) 1.90 g (87%) of the desired title compound.

¹H-NMR (400 MHz, DMSO d₆) δ (ppm)=2.74 (s, 3H), 5.64 (s, 2H), 7.88-7.97 (m, 2H).

### Intermediate 75B

### 1-[4-(methoxymethyl)benzyl]-5-methyl-4-nitro-3-(trifluoromethyl)-1H-pyrazole

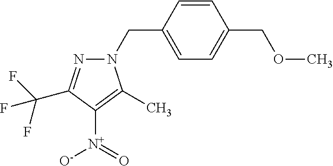 [SMILES: COCc1ccc(Cn2nc(C(F)(F)F)c([N+](=O)[O-])c2C)cc1]

To 500 mg (1.32 mmol) of 1-[4-(bromomethyl)benzyl]-3,5-dimethyl-4-nitro-1H-pyrazole from step 1 intermediate 71B was given a solution of 179 mg (3.31 mmol) sodium methylate in 17 mL methanol. This mixture was stirred for 15 hours at room temperature. The reaction mixtures was diluted with 50 mL ethyl acetate and this resulting organic phase was washed with water. After separation of the phases the aqueous phase was extracted once with ethyl acetate and the combined organic phases were dried over sodium sulfate, filtered and evaporated to dryness. The raw material was purified via a Biotage chromatography system (25 g snap KP-Sil column, hexane/0-100% ethyl acetate, then ethyl acetate/0-100% methanol) to obtain 380 mg (85%) of the desired title compound.

¹H-NMR (400 MHz, DMSO d₆) δ (ppm)=2.65 (s, 3H), 3.28 (s, 3H), 4.40 (s, 2H), 5.55 (s, 2H), 7.24 (d, 2H), 7.33 (d, 2H).

### Intermediate 76B

### (4-{[5-methyl-4-nitro-3-(trifluoromethyl)-1H-pyrazol-1-yl]methyl}phenyl)acetonitrile

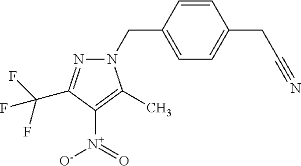 [SMILES: Cc1c([N+](=O)[O-])c(C(F)(F)F)nn1Cc1ccc(CC#N)cc1]

To a solution of 1.00 g (2.64 mmol) of 1-[4-(bromomethyl)benzyl]-3,5-dimethyl-4-nitro-1H-pyrazole from step 1 intermediate 71B in 15 mL DMSO was given 156 mg (3.17 mmol) sodium cyanide. This mixture was stirred for 2 hours at 40° C. After cooling to room temperature the reaction mixtures was diluted with 150 mL ethyl acetate and this resulting organic phase was washed with water. After separation of the phases the aqueous phase was extracted once with ethyl acetate and the combined organic phases were dried over sodium sulfate, filtered and evaporated to dryness. The raw material was purified via a Biotage chromatography system (25 g snap KP-Sil column, hexane/0-100% ethyl acetate, then ethyl acetate/0-100% methanol) to obtain 780 mg (86%) of the desired title compound.

¹H-NMR (400 MHz, DMSO d₆) δ (ppm)=2.66 (s, 3H), 4.04 (s, 2H), 5.56 (s, 2H), 7.29 (d, 2H), 7.37 (d, 2H).

### Intermediate 77B

### 3-[(3,5-dimethyl-4-nitro-1H-pyrazol-1-yl)methyl]-N-methyl-1,2,4-oxadiazole-5-carboxamide

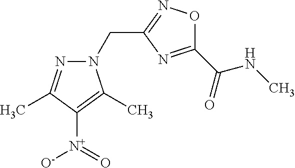

In analogy to intermediate 1B, 549 mg (3.89 mmol) 3,5-dimethyl-4-nitro-1H-pyrazole was heated with 820 mg (4.67 mmol) 3-(chloromethyl)-N-methyl-1,2,4-oxadiazole-5-carboxamide (CAS-No. 1123169-42-5) and 1.90 g (5.84 mmol) cesium carbonate in 20 mL acetonitrile for 2 h at 60° C. Afterwards the reaction mixture was filtered, the filtrate was evaporated, the residue was dissolved in dichloromethane and evaporated. The residue was purified by flash chromatography to yield 621 mg (2.11 mmol, 54%) of the desired title compound.

¹H NMR (300 MHz, DMSO-d₆): δ (ppm)=2.39 (s, 3H), 2.67 (s, 3H), 2.77 (s, 3H), 5.69 (s, 2H), 9.27 (br. s., 1H).

### Intermediate 78B

### tert-butyl 4-[(3,5-dimethyl-4-nitro-1H-pyrazol-1-yl)methyl]piperidine-1-carboxylate

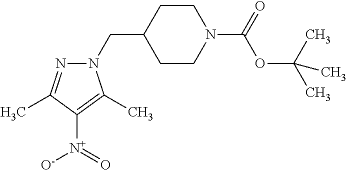 [SMILES: Cc1nn(CC2CCN(C(=O)OC(C)(C)C)CC2)c(C)c1[N+](=O)[O-]]

2.11 g (14.98 mmol) 3,5-Dimethyl-4-nitro-1H-pyrazole were heated with 5 g (17.97 mmol) tert-butyl 4-(bromomethyl)piperidine-1-carboxylate (CAS-No. 158407-04-6) and 3.35 mL (22.47 mmol) 1,8-diazabicyclo(5.4.0)undec-7-ene in 50 mL DMSO to 60° C. overnight. Water was added to the reaction mixture, and extracted with ethyl acetate. The combined organic phase was washed with water and brine, dried, filtered, and evaporated. The crude title compound (5.07 g, 94%) was used without further purification.

¹H NMR (400 MHz, DMSO-d₆): δ (ppm)=1.11 (dddd, 2H), 1.38 (s, 9H), 1.46 (br. d., 2H), 1.99 (m, 1H), 2.39 (s, 3H), 2.57 (s, 3H), 2.65 (m, 2H), 3.92 (br. d., 2H), 3.98 (d, 2H).

### Intermediate 79B

### 5-methyl-3-{[5-methyl-4-nitro-3-(trifluoromethyl)-1H-pyrazol-1-yl]methyl}-1,2-oxazole

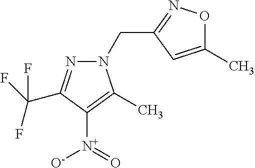 [SMILES: Cc1cc(Cn2nc(C(F)(F)F)c([N+](=O)[O-])c2C)no1]

In analogy to intermediate 78B, 6.15 g (31.5 mmol) 5-methyl-4-nitro-3-(trifluoromethyl)-1H-pyrazole and 4.98 g (37.9 mmol) 3-(chloromethyl)-5-methyl-1,2-oxazole (CAS-No. 35166-37-1) were reacted to give after purification of the crude product by flash chromatography 8.55 g (26.5 mmol, 84%) of the desired title compound.

¹H NMR (400 MHz, DMSO-d₆): δ (ppm)=2.39 (s, 3H), 2.67 (s, 3H), 5.64 (s, 2H), 6.25 (s, 1H).

### Intermediate 80B

### 5-ethyl-3-{[5-methyl-4-nitro-3-(trifluoromethyl)-1H-pyrazol-1-yl]methyl}-1,2,4-oxadiazole

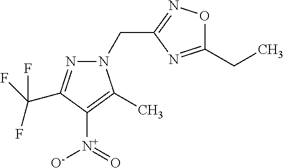 [SMILES: CCc1nc(Cn2nc(C(F)(F)F)c([N+](=O)[O-])c2C)no1]

In analogy to intermediate 78B, 2.22 g (11.4 mmol) 5-methyl-4-nitro-3-(trifluoromethyl)-1H-pyrazole and 2.00 g (13.6 mmol) 3-(chloromethyl)-5-ethyl-1,2,4-oxadiazole (CAS-No. 83227-01-4) were reacted to give after purification of the crude product by flash chromatography 2.35 g (7.30 mmol, 64%) of the desired title compound.

¹H NMR (400 MHz, DMSO-d₆): δ (ppm)=1.25 (t, 3H), 2.71 (s, 3H), 2.95 (q, 2H), 5.80 (s, 2H).

### Intermediate 81B

### 3-ethyl-5-{[5-methyl-4-nitro-3-(trifluoromethyl)-1H-pyrazol-1-yl]methyl}-1,2-oxazole

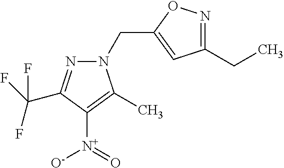 [SMILES: CCc1cc(Cn2nc(C(F)(F)F)c([N+](=O)[O-])c2C)on1]

In analogy to intermediate 78B, 1.12 g (5.72 mmol) 5-methyl-4-nitro-3-(trifluoromethyl)-1H-pyrazole and 1.00 g (6.87 mmol) 5-(chloromethyl)-3-ethyl-1,2-oxazole (CAS-No. 64988-69-8) were reacted to give after purification of the crude product by flash chromatography 690 mg (2.15 mmol, 38%) of the desired title compound.

¹H NMR (300 MHz, DMSO-d₆): δ (ppm)=1.16 (t, 3H), 2.61 (q, 2H), 2.70 (s, 3H), 5.78 (s, 2H), 6.52 (s, 1H).

### Intermediate 82B

### 5-[(3,5-dimethyl-4-nitro-1H-pyrazol-1-yl)methyl]-3-methyl-1,2-oxazole

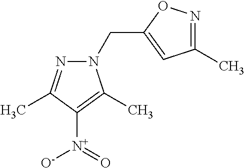

In analogy to intermediate 78B, 393 mg (2.79 mmol) 3,5-dimethyl-4-nitro-1H-pyrazole and 440 mg (3.34 mmol) 5-(chloromethyl)-3-methyl-1,2-oxazole (CAS-No. 40340-41-8) were reacted to give after purification of the crude product by flash chromatography 525 mg (2.11 mmol, 76%) of the desired title compound.

¹H NMR (300 MHz, DMSO-d₆): δ (ppm)=2.20 (s, 3H), 2.39 (s, 3H), 2.64 (s, 3H), 5.56 (s, 2H), 6.36 (s, 1H).

### Intermediate 83B

### 3-[(3,5-dimethyl-4-nitro-1H-pyrazol-1-yl)methyl]-5-methyl-1,2-oxazole

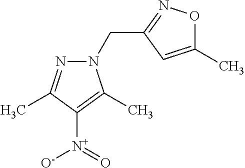

In analogy to intermediate 78B, 8.94 g (63.3 mmol) 3,5-dimethyl-4-nitro-1H-pyrazole and 10 g (76.0 mmol) 3-(chloromethyl)-5-methyl-1,2-oxazole (CAS-No. 35166-37-1) were reacted to give after purification of the crude product by flash chromatography 9.38 g (37.7 mmol, 60%) of the desired title compound.

¹H NMR (400 MHz, DMSO-d₆): δ (ppm)=2.37 (s, 3H), 2.39 (s, 3H), 2.61 (s, 3H), 5.42 (s, 2H), 6.16 (s, 1H).

### Intermediate 84B

### tert-butyl 4-{[5-methyl-4-nitro-3-(trifluoromethyl)-1H-pyrazol-1-yl]methyl}piperidine-1-carboxylate

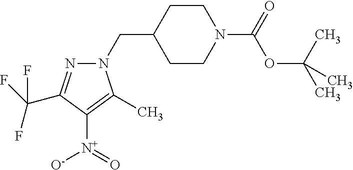 [SMILES: Cc1c([N+](=O)[O-])c(C(F)(F)F)nn1CC1CCN(C(=O)OC(C)(C)C)CC1]

In analogy to intermediate 78B, 2.38 g (11.98 mmol) 5-methyl-4-nitro-3-(trifluoromethyl)-1H-pyrazole and 4 g (14.38 mmol) tert-butyl 4-(bromomethyl)piperidine-1-carboxylate (CAS-No. 158407-04-6) were reacted to give after purification of the crude product by flash chromatography 3.67 g (8.89 mmol, 74%) of the desired title compound.

¹H NMR (300 MHz, CDCl₃): δ (ppm)=1.24 (dddd, 2H), 1.46 (s, 9H), 1.57 (br. d., 2H), 2.13 (m, 1H), 2.68 (s, 3H), 2.69 (m, 2H), 4.03 (d, 2H), 4.16 (m, 2H).

### Intermediate 85B

### 5-[(3,5-dimethyl-4-nitro-1H-pyrazol-1-yl)methyl]-3-ethyl-1,2-oxazole

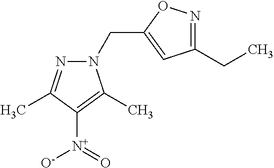 [SMILES: CCc1cc(Cn2nc(C)c([N+](=O)[O-])c2C)on1]

In analogy to intermediate 78B, 808 mg (5.72 mmol) 3,5-dimethyl-4-nitro-1H-pyrazole and 1.00 g (6.87 mmol) 5-(chloromethyl)-3-ethyl-1,2-oxazole (CAS-No. 64988-69-8) were reacted to give after purification of the crude product by flash chromatography 1.34 g (4.82 mmol, 84%) of the desired title compound.

¹H NMR (300 MHz, DMSO-d₆): δ (ppm)=1.16 (t, 3H), 2.39 (s, 3H), 2.60 (q, 2H), 2.64 (s, 3H), 5.56 (s, 2H), 6.41 (s, 1H).

### Intermediate 86B

### 4-[(3,5-dimethyl-4-nitro-1H-pyrazol-1-yl)methyl]piperidine

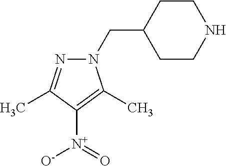 [SMILES: Cc1nn(CC2CCNCC2)c(C)c1[N+](=O)[O-]]

A solution of 5.28 g (14.0 mmol) tert-butyl 4-[(3,5-dimethyl-4-nitro-1H-pyrazol-1-yl)methyl]piperidine-1-carboxylate (intermediate 78B) in 125 mL dichloromethane was stirred with 10.8 mL (140.4 mmol) trifluoroacetic acid for 4.5 hours. The reaction mixture was filtered over NH₂ derivatized silica gel, and the filtrate was evaporated yielding 3.36 g of the desired title compound as crude product which was used without further purification.

¹H NMR (400 MHz, DMSO-d₆): δ (ppm)=1.11 (dddd, 2H), 1.42 (d, 2H), 1.88 (m, 1H), 2.39 (s, 3H), 2.57 (s, 3H), 2.40 (m, 2H), 2.92 (m, 2H), 3.94 (d, 2H).

### Intermediate 87B

### 4-[(3,5-dimethyl-4-nitro-1H-pyrazol-1-yl)methyl]-1-(ethylsulfonyl)piperidine

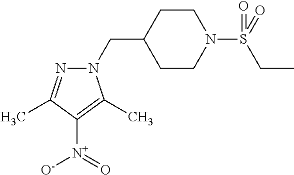 [SMILES: CCS(=O)(=O)N1CCC(Cn2nc(C)c([N+](=O)[O-])c2C)CC1]

A solution of 3.36 g (crude, −12.7 mmol) 4-[(3,5-dimethyl-4-nitro-1H-pyrazol-1-yl)methyl]piperidine (intermediate 86B) in 30 mL DMF was stirred with 1.68 mL (17.8 mmol) ethanesulfonyl chloride and 10.6 mL (76.1 mmol) triethylamine overnight. Saturated aqueous sodium bicarbonate and ethyl acetate were added to the reaction. The mixture was extracted with butanol, and the combined organic phase was washed with brine, dried, filtered, and evaporated. Purification by flash chromatography yielded 2.53 g (57%) of the desired title compound.

¹H NMR (400 MHz, DMSO-d₆): δ (ppm)=1.19 (t, 3H), 1.26 (dddd, 2H), 1.57 (m, 2H), 1.97 (m, 1H), 2.40 (s, 3H), 2.58 (s, 3H), 2.75 (m, 2H), 3.00 (q, 2H), 3.58 (m, 2H), 3.90 (d, 2H), 4.02 (s, 2H).

### Intermediate 88B

### 5-[(3,5-dimethyl-4-nitro-1H-pyrazol-1-yl)methyl]-3-(propan-2-yl)-1,2-oxazole

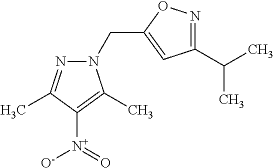 [SMILES: Cc1nn(Cc2cc(C(C)C)no2)c(C)c1[N+](=O)[O-]]

In analogy to intermediate 78B, 737 mg (5.22 mmol) 3,5-dimethyl-4-nitro-1H-pyrazole and 1.00 g (6.27 mmol) 5-(chloromethyl)-3-(propan-2-yl)-1,2-oxazole (CAS-No. 64988-71-2) were reacted to give after purification of the crude product by flash chromatography 1.07 g (3.64 mmol, 70%) of the desired title compound.

¹H NMR (300 MHz, DMSO-d₆): δ (ppm)=1.19 (d, 6H), 2.39 (s, 3H), 2.65 (s, 3H), 2.96 (sept, 1H), 5.56 (s, 2H), 6.47 (s, 1H).

### Intermediate 89B

### 5-cyclopropyl-3-[(3,5-dimethyl-4-nitro-1H-pyrazol-1-yl)methyl]-1,2-oxazole

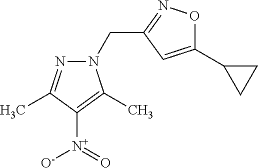 [SMILES: Cc1nn(Cc2cc(C3CC3)on2)c(C)c1[N+](=O)[O-]]

In analogy to intermediate 78B, 746 mg (5.29 mmol) 3,5-dimethyl-4-nitro-1H-pyrazole and 1 g (6.35 mmol) 3-(chloromethyl)-5-cyclopropyl-1,2-oxazole (CAS-No. 1060817-59-5) were reacted to give after purification of the crude product by flash chromatography 1.31 g (4.81 mmol, 91%) of the desired title compound.

¹H NMR (400 MHz, DMSO-d₆): δ (ppm)=0.86 (m, 2H), 1.04 (m, 2H), 2.11 (m, 1H), 2.39 (s, 3H), 2.60 (s, 3H), 5.39 (s, 2H), 6.12 (s, 1H).

### Intermediate 90B

### 3-methyl-5-{[5-methyl-4-nitro-3-(trifluoromethyl)-1H-pyrazol-1-yl]methyl}-1,2-oxazole

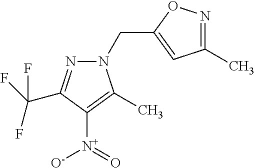 [SMILES: Cc1cc(Cn2nc(C(F)(F)F)c([N+](=O)[O-])c2C)on1]

In analogy to intermediate 78B, 1.17 mg (6.02 mmol) 5-methyl-4-nitro-3-(trifluoromethyl)-1H-pyrazole and 1 g (7.22 mmol) 5-(chloromethyl)-3-methyl-1,2-oxazole (CAS-No. 40340-41-8) were reacted to give after purification of the crude product by flash chromatography 1.40 g (4.58 mmol, 76%) of the desired title compound.

¹H NMR (300 MHz, DMSO-d₆): δ (ppm)=2.21 (s, 3H), 2.70 (s, 3H), 5.77 (s, 2H), 6.45 (s, 1H).

### Intermediate 91B

### 5-[(3,5-dimethyl-4-nitro-1H-pyrazol-1-yl)methyl]thiophene-2-carbonitrile

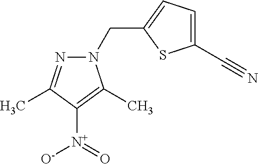 [SMILES: Cc1nn(Cc2ccc(C#N)s2)c(C)c1[N+](=O)[O-]]

In analogy to intermediate 78B, 291 mg (2.06 mmol) 3,5-dimethyl-4-nitro-1H-pyrazole and 500 mg (2.47 mmol) 5-(bromomethyl)thiophene-2-carbonitrile (CAS-No. 134135-41-4) were reacted to give after purification of the crude product by flash chromatography 472 mg (1.44 mmol, 58%) of the desired title compound.

¹H NMR (400 MHz, DMSO-d₆): δ (ppm)=2.41 (s, 3H), 2.63 (s, 3H), 5.67 (s, 2H), 7.26 (d, 1H), 7.86 (d, 1H).

### Intermediate 92B

### 5-{[5-methyl-4-nitro-3-(trifluoromethyl)-1H-pyrazol-1-yl]methyl}thiophene-2-carbonitrile

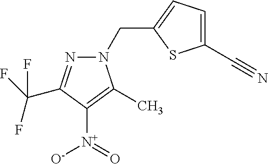 [SMILES: Cc1c([N+](=O)[O-])c(C(F)(F)F)nn1Cc1ccc(C#N)s1]

In analogy to intermediate 78B, 402 mg (2.06 mmol) 5-methyl-4-nitro-3-(trifluoromethyl)-1H-pyrazole and 500 mg (2.47 mmol) 5-(bromomethyl)thiophene-2-carbonitrile (CAS-No. 134135-41-4) were reacted to give after purification of the crude product by flash chromatography 630 mg (1.79 mmol, 72%) of the desired title compound.

¹H NMR (400 MHz, DMSO-d₆): δ (ppm)=2.70 (s, 3H), 5.87 (s, 2H), 7.31 (d, 1H), 7.90 (d, 1H).

### Intermediate 93B

### 2-{[5-methyl-4-nitro-3-(trifluoromethyl)-1H-pyrazol-1-yl]methyl}pyrimidine-5-carbonitrile

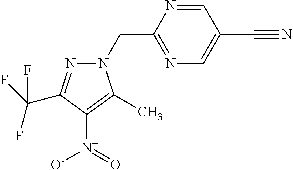 [SMILES: Cc1c([N+](=O)[O-])c(C(F)(F)F)nn1Cc1ncc(C#N)cn1]

In analogy to intermediate 1B), 2.05 g (10.5 mmol) 5-methyl-4-nitro-3-(trifluoromethyl)-1H-pyrazole and 2.50 g (12.6 mmol) 2-(bromomethyl)pyrimidine-5-carbonitrile (commercially available e.g. ABCR) were reacted to give after purification of the crude product mixture via a Biotage chromatography system (25 g snap KP-Sil column, hexane/0-100% ethyl acetate, then ethyl acetate/0-25% methanol) 2.9 g (86%) of the desired compound.

¹H-NMR (400 MHz, DMSO d₆) δ (ppm)=2.61 (s, 3H), 5.97 (s, 2H), 9.30 (s, 2H).

### Intermediate 94B

### 6-{[5-methyl-4-nitro-3-(trifluoromethyl)-1H-pyrazol-1-yl]methyl}pyridazine-3-carbonitrile

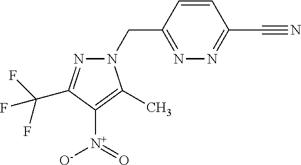 [SMILES: Cc1c([N+](=O)[O-])c(C(F)(F)F)nn1Cc1ccc(C#N)nn1]

Step 1: 6-(chloromethyl)pyridazine-3-carbonitrile

To a solution of 250 mg (2.10 mmol) 6-methylpyridazine-3-carbonitrile in 20 mL dichloromethane was added 161 mg (0.69 mmol) trichloroisocyanuric acid and this reaction mixture was heated up to 90° C. for 5 hours. After cooling to 25° C. this mixture was evaporated to dryness, getting a raw 6-(chloromethyl)pyridazine-3-carbonitrile, which was used without any further purification in the following step. In a second experiment this reaction was repeated using 1.75 g (14.7 mmol) 6-methylpyridazine-3-carbonitrile yielding 2.5 g raw 6-(chloromethyl)pyridazine-3-carbonitrile.

### Step 2:

In analogy to intermediate 1B), in a first experiment 322 mg and in a second experiment 2.5 g of the raw material of step 1) were reacted with 341 mg (1.78 mmol) and in the second experiment with 1.68 (8.58 mmol) 5-methyl-4-nitro-3-(trifluoromethyl)-1H-pyrazole to give after three subsequent purifications of the combined crude products via a Biotage chromatography system (1. 50 g snap KP-Sil column, hexane/0-100% ethyl acetate, then ethyl acetate/0-100% methanol/2. 25 g snap KP-Sil column, hexane/0-100% ethyl acetate, then ethyl acetate/0-25% methanol/3. 25 g snap KP-Sil column, hexane/0-100% ethyl acetate) 0.63 g (9.6%, purity about 40%) of the desired title compound.

¹H NMR (400 MHz, CDCl₃): δ (ppm)=6.06 (s, 2H), 8.00 (d, 1H), 8.42 (d, 1H).

### Intermediate 1C

### 1-(4-fluorobenzyl)-3,5-dimethyl-1H-pyrazol-4-amine

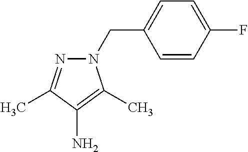 [SMILES: Cc1nn(Cc2ccc(F)cc2)c(C)c1N]

9.33 g (37.43 mmol) 1-(4-fluorobenzyl)-3,5-dimethyl-4-nitro-1H-pyrazole (intermediate 1B) was dissolved in 250 mL methanol and 1.99 g (1.87 mmol) palladium on carbon (10 wt. %) and 23.6 g (374.3 mmol) ammonium formate were added. The reaction mixture was heated for 1 h at 80° C. Afterwards the suspension was filtered through Celite and the filtrate was evaporated. The residue was partitioned between water and ethyl acetate. The layers were separated and the organic layer was washed with brine, dried over sodium sulfate, filtered and evaporated to obtain 7.29 g (33.25 mmol, 89%) of the desired title compound after drying.

1H NMR (300 MHz, CDCl₃): δ (ppm)=2.05 (s, 3H), 2.20 (s, 3H), 2.56 (br. s., 2H), 5.13 (s, 2H), 6.86-7.12 (m, 4H).

### Intermediate 2C

### 1-(3-fluorobenzyl)-3,5-dimethyl-1H-pyrazol-4-amine

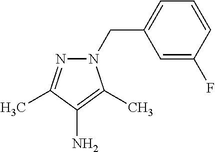 [SMILES: Cc1nn(Cc2cccc(F)c2)c(C)c1N]

In analogy to intermediate 1C), 682 mg (2.74 mmol) 1-(3-fluorobenzyl)-3,5-dimethyl-4-nitro-1H-pyrazole (intermediate 2B) was heated with 145 mg (0.14 mmol) palladium on carbon (10 wt. %) and 1.38 g (21.90 mmol) ammonium formate in 5 mL methanol for 1 h at 80° C. to obtain 454 mg (2.07 mmol, 76%) of the desired title compound after filtration and aqueous work-up.

1H NMR (400 MHz, CDCl₃): δ (ppm)=2.06 (s, 3H), 2.21 (s, 3H), 2.54 (br. s., 2H), 5.16 (s, 2H), 6.72 (d, 1H), 6.84 (d, 1H), 6.93 (td, 1H), 7.22-7.26 (m, 1H).

### Intermediate 3C

### 1-(2-fluorobenzyl)-3,5-dimethyl-1H-pyrazol-4-amine

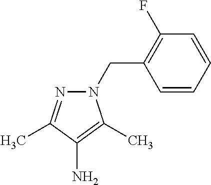 [SMILES: Cc1nn(Cc2ccccc2F)c(C)c1N]

In analogy to intermediate 1C), 336 mg (1.35 mmol) 1-(2-fluorobenzyl)-3,5-dimethyl-4-nitro-1H-pyrazole (intermediate 3B) was heated with 71 mg (0.07 mmol) palladium on carbon (10 wt. %) and 680 mg (10.78 mmol) ammonium formate in 25 mL methanol for 1 h at 80° C. to obtain 275 mg (1.25 mmol, 93%) of the desired title compound after filtration and aqueous work-up.

1H NMR (400 MHz, CDCl₃): δ (ppm)=2.09 (s, 3H), 2.20 (s, 3H), 5.22 (s, 2H), 6.79-6.86 (m, 1H), 7.03-7.09 (m, 2H), 7.19-7.26 (m, 1H).

### Intermediate 4C

### 1-(3,4-difluorobenzyl)-3,5-dimethyl-1H-pyrazol-4-amine

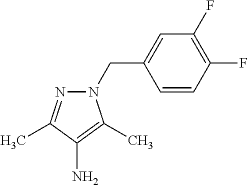 [SMILES: Cc1nn(Cc2ccc(F)c(F)c2)c(C)c1N]

In analogy to intermediate 1C), 726 mg (2.72 mmol) 1-(3,4-difluorobenzyl)-3,5-dimethyl-4-nitro-1H-pyrazole (intermediate 4B) was heated with 144 mg (0.14 mmol) palladium on carbon (10 wt. %) and 1.37 g (21.73 mmol) ammonium formate in 25 mL methanol for 1 h at 80° C. to obtain 644 mg (2.20 mmol, 81%) of the desired title compound after filtration and aqueous work-up.

Method 1: Rₜ=0.65 min

MS (ESIpos): m/z=238 (M+H)⁺.

### Intermediate 5C

### 1-(2,4-difluorobenzyl)-3,5-dimethyl-1H-pyrazol-4-amine

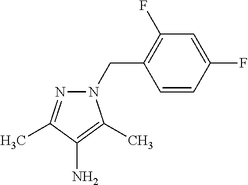 [SMILES: Cc1nn(Cc2ccc(F)cc2F)c(C)c1N]

In analogy to intermediate 1C), 716 mg (2.68 mmol) 1-(2,4-difluorobenzyl)-3,5-dimethyl-4-nitro-1H-pyrazole (intermediate 5B) was heated with 142 mg (0.13 mmol) palladium on carbon (10 wt. %) and 1.35 g (21.43 mmol) ammonium formate in 25 mL methanol for 1 h at 80° C. to obtain 635 mg (2.67 mmol, 99%) of the desired title compound after filtration and aqueous work-up.

Method 1: Rₜ=0.64 min

MS (ESIpos): m/z=238 (M+H)⁺.

### Intermediate 6C

### 1-(2,6-difluorobenzyl)-3,5-dimethyl-1H-pyrazol-4-amine

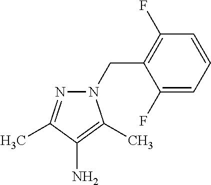 [SMILES: Cc1nn(Cc2c(F)cccc2F)c(C)c1N]

In analogy to intermediate 1C), 719 mg (2.69 mmol) 1-(2,6-difluorobenzyl)-3,5-dimethyl-4-nitro-1H-pyrazole (intermediate 6B) was heated with 143 mg (0.13 mmol) palladium on carbon (10 wt. %) and 1.36 g (21.52 mmol) ammonium formate in 20 mL methanol for 1 h at 80° C. to obtain 644 mg (2.20 mmol, 81%) of the desired title compound after filtration and aqueous work-up.

Method 1: Rₜ=0.63 min

MS (ESIpos): m/z=238 (M+H)⁺.

### Intermediate 7C

### 3,5-dimethyl-1-(2,4,6-trifluorobenzyl)-1H-pyrazol-4-amine

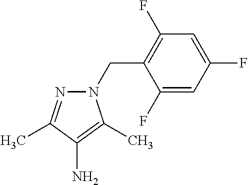 [SMILES: Cc1nn(Cc2c(F)cc(F)cc2F)c(C)c1N]

In analogy to intermediate 1C), 708 mg (2.48 mmol) 3,5-dimethyl-4-nitro-1-(2,4,6-trifluorobenzyl)-1H-pyrazole (intermediate 7B) was heated with 132 mg (0.12 mmol) palladium on carbon (10 wt. %) and 1.25 g (19.86 mmol) ammonium formate in 25 mL methanol for 1 h at 80° C. to obtain 602 mg (2.33 mmol, 94%) of the desired title compound after filtration and aqueous work-up.

Method 1: Rₜ=0.64 min

MS (ESIpos): m/z=256 (M+H)⁺.

### Intermediate 8C

### 4-[(4-amino-3,5-dimethyl-1H-pyrazol-1-yl)methyl]benzonitrile

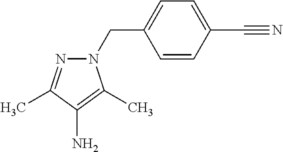 [SMILES: Cc1nn(Cc2ccc(C#N)cc2)c(C)c1N]

To a solution of 2.25 g (8.78 mmol) 4-[(3,5-dimethyl-4-nitro-1H-pyrazol-1-yl)methyl]benzonitrile (intermediate 8B) in 100 mL ethanol was added 50 mL water, 10 mL acetic acid and 2.01 g (30.7 mmol) zinc dust. This reaction mixture was stirred at 60° C. for 2 hours. After cooling to 25° C. the suspension was filtered through Celite, washed with ethyl acetate and the complete filtrate was evaporated. To the residue was added 100 mL water and 30 mL of conc. aq. sodium carbonate. This aqueous phase was extracted three times with 100 mL ethyl acetate. The combined organic layer was washed with brine, dried over sodium sulfate, filtered and evaporated to obtain a crude product, which was purified via a Biotage chromatography system (50 g snap KP-Sil column, ethyl acetate/0-50% methanol) to obtain 1.77 g (89%) of the desired title compound.

¹H-NMR (300 MHz, DMSO d₆) δ (ppm)=1.97 (s, 3H), 1.99 (s, 3H), 3.43 (br. s., 2H), 5.17 (s, 2H), 7.10-7.21 (m, 2H), 7.77 (d, 2H).

### Intermediate 9C

### 3-[(4-amino-3,5-dimethyl-1H-pyrazol-1-yl)methyl]benzonitrile

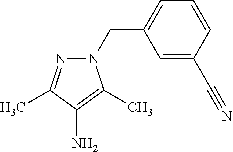 [SMILES: Cc1nn(Cc2cccc(C#N)c2)c(C)c1N]

To a solution of 500 mg (1.95 mmol) 3-[(3,5-dimethyl-4-nitro-1H-pyrazol-1-yl)methyl]benzonitrile (intermediate 9B) in 10 mL ethanol was added 1.85 g (9.76 mmol) tin(II) chloride. This reaction mixture was stirred at 78° C. for 8 hours. After cooling to room temperature the reaction mixture was brought to pH 8 via addition of 2 M sodium hydroxide solution. The resulting precipitate was isolated by filtration and the filtrate extracted with dichloromethane. The combined organic layers were washed with brine, dried over sodium sulfate, filtered and evaporated to obtain 424 mg (1.59 mmol, 82%) of the desired title compound.

### Intermediate 10C

### 2-[(4-amino-3,5-dimethyl-1H-pyrazol-1-yl)methyl]benzonitrile

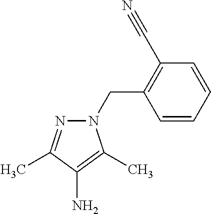 [SMILES: Cc1nn(Cc2ccccc2C#N)c(C)c1N]

To a solution of 2.48 g (9.68 mmol) 2-[(3,5-dimethyl-4-nitro-1H-pyrazol-1-yl)methyl]benzonitrile (intermediate 10B) in 51 mL ethanol was added 10.9 g (48.4. mmol) stannous chloride dihydrate. This reaction mixture was stirred at reflux for 5 hours and then at 70° C. for 20 hours. After cooling to 25° C. the mixture was evaporated. To the residue was added 5M aq. sodium hydroxide solution to get a basic pH. This aqueous phase was extracted three times with 80 mL ethyl acetate. The combined organic layer was washed with water, brine, dried over sodium sulfate, filtered and evaporated to obtain a crude product, which was purified via a Biotage chromatography system (50 g snap KP-Sil column, ethyl acetate/0-35% methanol) to obtain 1.91 g (83%) of the desired title compound.

¹H-NMR (400 MHz, DMSO d₆) δ (ppm)=1.98 (s, 3H), 2.04 (s, 3H), 3.42 (br. s., 2H), 5.24 (s, 2H), 6.86 (d, 1H), 7.42-7.50 (m, 1H), 7.62 (td, 1H), 7.83 (dd, 1H).

### Intermediate 11C

### 1-(4-methoxybenzyl)-3,5-dimethyl-1H-pyrazol-4-amine

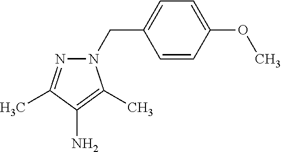

In analogy to intermediate 1C), 2.41 g (9.22 mmol) 1-(4-methoxybenzyl)-3,5-dimethyl-4-nitro-1H-pyrazole (intermediate 11B) were reacted to give after purification of the crude product via a Biotage chromatography system (50 g snap KP-Sil column, hexane/20-70% ethyl acetate) 2.35 g (105%) of the desired, not completely pure compound, which was used without any further purification.

¹H-NMR (400 MHz, DMSO d₆) δ (ppm)=1.98 (s, 6H), 3.65 (br. s., 2H), 3.70 (s, 3H), 4.97 (s, 2H), 6.84 (d, 2H), 6.99 (d, 2H).

### Intermediate 12C

### 1-(3-methoxybenzyl)-3,5-dimethyl-1H-pyrazol-4-amine

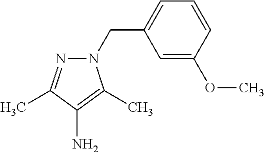

In analogy to intermediate 1C), 339 mg (1.30 mmol) 1-(3-methoxybenzyl)-3,5-dimethyl-4-nitro-1H-pyrazole (intermediate 12B) was heated with 69 mg (0.07 mmol) palladium on carbon (10 wt. %) and 654 mg (10.38 mmol) ammonium formate in 5 mL methanol for 1 h at 80° C. to obtain 294 mg (1.27 mmol, 98%) of the desired title compound after filtration and aqueous work-up.

1H NMR (300 MHz, CDCl₃): δ (ppm)=2.06 (s, 3H), 2.20 (s, 3H), 2.51 (br. s., 2H), 3.76 (s, 3H), 5.15 (s, 2H), 6.55-6.71 (m, 2H), 6.73-6.84 (m, 1H), 7.21 (t, 1H).

### Intermediate 13C

### 3,5-dimethyl-1-(4-methylbenzyl)-1H-pyrazol-4-amine

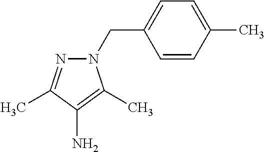

In analogy to intermediate 1C), 686 mg (2.80 mmol) 3,5-dimethyl-1-(4-methylbenzyl)-4-nitro-1H-pyrazole (intermediate 13B) was heated with 149 mg (0.14 mmol) palladium on carbon (10 wt. %) and 1.41 g (22.37 mmol) ammonium formate in 25 mL methanol for 1 h at 80° C. to obtain 550 mg (2.53 mmol, 90%) of the desired title compound after filtration and aqueous work-up.

Method 1: Rₜ=0.69 min

MS (ESIpos): m/z=216 (M+H)⁺.

### Intermediate 14C

### 3,5-dimethyl-1-(3-methylbenzyl)-1H-pyrazol-4-amine

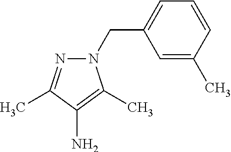

In analogy to intermediate 1C), 681 mg (2.78 mmol) 3,5-dimethyl-1-(3-methylbenzyl)-4-nitro-1H-pyrazole (intermediate 14B) was heated with 147 mg (0.14 mmol) palladium on carbon (10 wt. %) and 1.40 g (22.21 mmol) ammonium formate in 25 mL methanol for 1 h at 80° C. to obtain 541 mg (2.49 mmol, 90%) of the desired title compound after filtration and aqueous work-up.

Method 1: Rₜ=0.69 min

MS (ESIpos): m/z=216 (M+H)⁺.

### Intermediate 15C

### 3,5-dimethyl-1-(2-methylbenzyl)-1H-pyrazol-4-amine

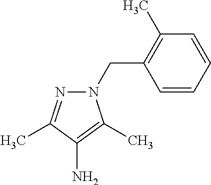 [SMILES: Cc1ccccc1Cn1nc(C)c(N)c1C]

In analogy to intermediate 1C), 695 mg (2.83 mmol) 3,5-dimethyl-1-(2-methylbenzyl)-4-nitro-1H-pyrazole (intermediate 15B) was heated with 151 mg (0.14 mmol) palladium on carbon (10 wt. %) and 1.43 g (22.67 mmol) ammonium formate in 25 mL methanol for 1 h at 80° C. to obtain 600 mg (2.60 mmol, 91%) of the desired title compound after filtration and aqueous work-up.

Method 1: Rₜ=0.69 min

MS (ESIpos): m/z=216 (M+H)⁺.

### Intermediate 16C

### 3,5-dimethyl-1-(pyridin-4-ylmethyl)-1H-pyrazol-4-amine

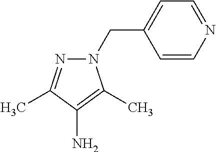

In analogy to intermediate 1C), 1.57 g (6.76 mmol) 4-[(3,5-dimethyl-4-nitro-1H-pyrazol-1-yl)methyl]pyridine (intermediate 16B) were reacted to give 0.38 g (22%) of the desired title compound.

¹H-NMR (300 MHz, DMSO d₆) δ (ppm)=1.97 (s, 3H), 1.99 (s, 3H), 5.12 (s, 2H), 6.94 (d, 2H), 8.44-8.49 (m, 2H).

### Intermediate 17C

### 3,5-dimethyl-1-(pyridin-3-ylmethyl)-1H-pyrazol-4-amine

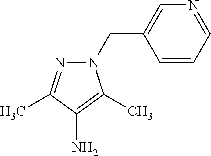

In analogy to intermediate 10C), 1.00 g (4.31 mmol) 3-[(3,5-dimethyl-4-nitro-1H-pyrazol-1-yl)methyl]pyridine (intermediate 17B) were reacted to give without purification 0.27 g (28%) of the desired title compound.

¹H-NMR (300 MHz, DMSO d₆) δ (ppm)=1.97-1.99 (m, 3H), 2.02 (s, 3H), 3.40 (br. s., 2H), 5.10 (s, 2H), 7.29-7.34 (m, 1H), 7.40 (dt, 1H), 8.31 (d, 1H), 8.44 (dd, 1H).

### Intermediate 18C

### 3,5-dimethyl-1-(pyridin-2-ylmethyl)-1H-pyrazol-4-amine

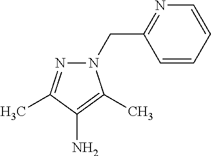

In analogy to intermediate 1C), 467 mg (2.01 mmol) 2-[(3,5-dimethyl-4-nitro-1H-pyrazol-1-yl)methyl]pyridine (intermediate 18B) was heated with 107 mg (0.10 mmol) palladium on carbon (10 wt. %) and 1.01 g (16.09 mmol) ammonium formate in 5 mL methanol for 1 h at 80° C. to obtain 327 mg (1.62 mmol, 80%) of the desired title compound after filtration and aqueous work-up.

1H NMR (300 MHz, CDCl₃): δ (ppm)=2.08 (s, 3H), 2.22 (s, 3H), 2.46 (br. s., 2H), 5.30 (s, 2H), 6.77 (d, 1H), 7.10-7.22 (m, 1H), 7.59 (td, 1H), 8.48-8.60 (m, 1H).

### Intermediate 19C

### 3,5-dimethyl-1-[4-(trifluoromethoxy)benzyl]-1H-pyrazol-4-amine

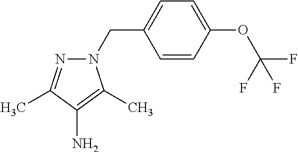 [SMILES: Cc1nn(Cc2ccc(OC(F)(F)F)cc2)c(C)c1N]

In analogy to intermediate 1C), 837 mg (2.66 mmol) 3,5-dimethyl-4-nitro-1-[4-(trifluoromethoxy)benzyl]-1H-pyrazole (intermediate 19B) was heated with 141 mg (0.13 mmol) palladium on carbon (10 wt. %) and 1.34 g (21.24 mmol) ammonium formate in 20 mL methanol for 1 h at 80° C. to obtain 750 mg (2.10 mmol, 79%) of the desired title compound after filtration and aqueous work-up.

Method 1: Rₜ=0.79 min

MS (ESIpos): m/z=286 (M+H)⁺.

### Intermediate 20C

### 3,5-dimethyl-1-[3-(trifluoromethoxy)benzyl]-1H-pyrazol-4-amine

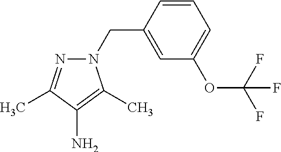 [SMILES: Cc1nn(Cc2cccc(OC(F)(F)F)c2)c(C)c1N]

In analogy to intermediate 1C), 707 mg (2.24 mmol) 3,5-dimethyl-4-nitro-1-[3-(trifluoromethoxy)benzyl]-1H-pyrazole (intermediate 20B) was heated with 119 mg (0.11 mmol) palladium on carbon (10 wt. %) and 1.13 g (17.94 mmol) ammonium formate in 20 mL methanol for 1 h at 80° C. to obtain 639 mg (1.43 mmol, 64%) of the desired title compound after filtration and aqueous work-up.

Method 1: Rₜ=0.79 min

MS (ESIpos): m/z=286 (M+H)⁺.

### Intermediate 21C

### 3,5-dimethyl-1-[2-(trifluoromethoxy)benzyl]-1H-pyrazol-4-amine

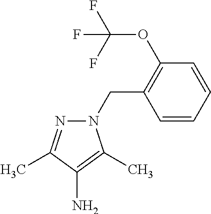 [SMILES: Cc1nn(Cc2ccccc2OC(F)(F)F)c(C)c1N]

In analogy to intermediate 1C), 893 mg (2.83 mmol) 3,5-dimethyl-4-nitro-1-[2-(trifluoromethoxy)benzyl]-1H-pyrazole (intermediate 21B) was heated with 150 mg (0.14 mmol) palladium on carbon (10 wt. %) and 1.43 g (22.66 mmol) ammonium formate in 20 mL methanol for 1 h at 80° C. to obtain 688 mg (1.33 mmol, 47%) of the desired title compound after filtration and aqueous work-up.

Method 1: Rₜ=0.78 min

MS (ESIpos): m/z=286 (M+H)⁺.

### Intermediate 22C

### 3,5-dimethyl-1-[4-(trifluoromethyl)benzyl]-1H-pyrazol-4-amine

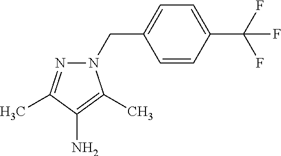 [SMILES: Cc1nn(Cc2ccc(C(F)(F)F)cc2)c(C)c1N]

In analogy to intermediate 1C), 790 mg (2.64 mmol) 3,5-dimethyl-4-nitro-1-[4-(trifluoromethyl)benzyl]-1H-pyrazole (intermediate 22B) was heated with 140 mg (0.13 mmol) palladium on carbon (10 wt. %) and 1.33 g (21.12 mmol) ammonium formate in 5 mL methanol for 1 h at 80° C. to obtain 653 mg (2.42 mmol, 92%) of the desired title compound after filtration and aqueous work-up.

1H NMR (300 MHz, CDCl₃): δ (ppm)=2.07 (s, 3H), 2.17-2.26 (m, 3H), 2.76 (br. s., 2H), 5.22 (s, 2H), 7.15 (m, 2H), 7.56 (m, 2H).

### Intermediate 23C

### 1-(3,4-difluorobenzyl)-5-methyl-3-(trifluoromethyl)-1H-pyrazol-4-amine

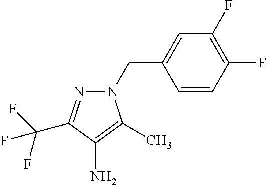 [SMILES: Cc1c(N)c(C(F)(F)F)nn1Cc1ccc(F)c(F)c1]

In analogy to intermediate 1C), in a first experiment 0.20 g (0.623 mmol) and in a second experiment 1.56 g (4.86 mmol) 1-(3,4-difluorobenzyl)-5-methyl-4-nitro-3-(trifluoromethyl)-1H-pyrazole (intermediate 23B) were reacted to give after combined purification of both crude products via a Biotage chromatography system (50 g snap KP-Sil column, hexane/40-100% ethyl acetate) 0.90 g (57%) of the desired title compound.

¹H-NMR (400 MHz, DMSO d₆) δ (ppm)=2.07 (s, 3H), 4.02 (s, 2H), 5.25 (s, 2H), 6.91 (ddd, 1H), 7.17 (ddd, 1H), 7.40 (dt, 1H).

### Intermediate 24C

### 1-(2,4-difluorobenzyl)-5-methyl-3-(trifluoromethyl)-1H-pyrazol-4-amine

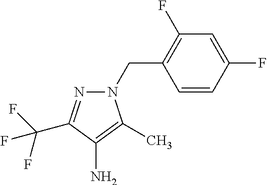 [SMILES: Cc1c(N)c(C(F)(F)F)nn1Cc1ccc(F)cc1F]

In analogy to intermediate 1C), 1.73 g (5.39 mmol) 1-(2,4-difluorobenzyl)-5-methyl-4-nitro-3-(trifluoromethyl)-1H-pyrazole (intermediate 24B) were reacted to give after purification of the crude products via a Biotage chromatography system (50 g snap KP-Sil column, hexane/40-100% ethyl acetate) 1.91 g (113%) of the desired, not completely pure compound, which was used without any further purification.

¹H-NMR (300 MHz, DMSO d₆) δ (ppm)=2.11 (s, 3H), 4.02 (s, 2H), 5.26 (s, 2H), 6.99-7.12 (m, 2H), 7.28 (td, 1H).

### Intermediate 25C

### 1-(4-fluorobenzyl)-5-methyl-3-(trifluoromethyl)-1H-pyrazol-4-amine

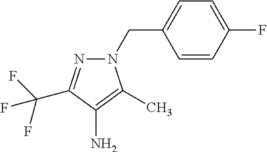 [SMILES: Cc1c(N)c(C(F)(F)F)nn1Cc1ccc(F)cc1]

In analogy to intermediate 1C), 4.21 g (13.9 mmol) 1-(4-fluorobenzyl)-5-methyl-4-nitro-3-(trifluoromethyl)-1H-pyrazole (intermediate 25B) were reacted to give after purification of the crude products via a Biotage chromatography system (100 g snap KP-Sil column, hexane/10-70% ethyl acetate) 3.37 g (76%) of the desired title compound.

¹H-NMR (400 MHz, DMSO d₆) δ (ppm)=2.06 (s, 3H), 3.99 (s, 2H), 5.24 (s, 2H), 7.13-7.21 (m, 4H).

### Intermediate 26C

### 4-{[4-amino-5-methyl-3-(trifluoromethyl)-1H-pyrazol-1-yl]methyl}benzonitrile

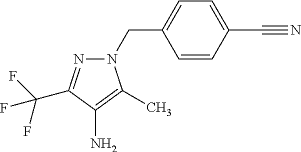 [SMILES: Cc1c(N)c(C(F)(F)F)nn1Cc1ccc(C#N)cc1]

In analogy to intermediate 8C), 3.20 g (10.3 mmol) 4-{[5-methyl-4-nitro-3-(trifluoromethyl)-1H-pyrazol-1-yl]methyl}benzonitrile (intermediate 26B) were reacted to give after purification of the crude products via a Biotage chromatography system (50 g snap KP-Sil column, hexane/30-80% ethyl acetate) 2.66 g (87%) of the desired title compound.

¹H-NMR (300 MHz, DMSO d₆) δ (ppm)=2.05 (s, 3H), 4.06 (s, 2H), 5.38 (s, 2H), 7.19-7.26 (m, 2H), 7.79-7.85 (m, 2H).

### Intermediate 27C

### 1-(4-chlorobenzyl)-3,5-dimethyl-1H-pyrazol-4-amine

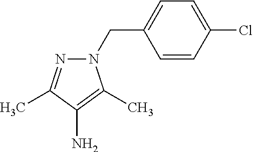 [SMILES: Cc1nn(Cc2ccc(Cl)cc2)c(C)c1N]

In analogy to intermediate 10C), 3.50 g (13.2 mmol) 1-(4-chlorobenzyl)-3,5-dimethyl-4-nitro-1H-pyrazole (intermediate 27B) were reacted to give without further purification 2.85 g (83%) of the desired, not completely pure compound.

¹H-NMR (300 MHz, DMSO d₆) δ (ppm)=1.97 (s, 3H), 1.98 (s, 3H), 3.38 (s, 2H), 5.06 (s, 2H), 7.03 (d, 2H), 7.33-7.38 (m, 2H).

### Intermediate 28C

### 1-(3-chloro-4-fluorobenzyl)-3,5-dimethyl-1H-pyrazol-4-amine

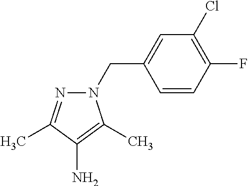 [SMILES: Cc1nn(Cc2ccc(F)c(Cl)c2)c(C)c1N]

In analogy to intermediate 10C), 1.50 g (5.29 mmol) 1-(3-chloro-4-fluorobenzyl)-3,5-dimethyl-4-nitro-1H-pyrazole (intermediate 28B) were reacted to give without further purification 1.24 g (74%) of the desired, not completely pure compound.

¹H-NMR (300 MHz, DMSO d₆) δ (ppm)=1.99 (s, 6H), 3.40 (s, 2H), 5.06 (s, 2H), 7.01 (ddd, 1H), 7.20 (dd, 1H), 7.30-7.39 (m, 1H).

### Intermediate 29C

### 5-[(4-amino-3,5-dimethyl-1H-pyrazol-1-yl)methyl]pyridine-2-carbonitrile

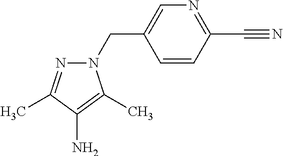 [SMILES: Cc1nn(Cc2ccc(C#N)nc2)c(C)c1N]

In analogy to intermediate 8C), 3.69 g (14.3 mmol) 5-[(3,5-dimethyl-4-nitro-1H-pyrazol-1-yl)methyl]pyridine-2-carbonitrile (intermediate 29B) were reacted to give after purification of the crude products via a Biotage chromatography system (50 g snap KP-Sil column, ethyl acetate/0-50% methanol) 1.00 g (28%) of the desired title compound.

¹H-NMR (300 MHz, DMSO d₆) δ (ppm)=1.98 (s, 3H), 2.02 (s, 3H), 3.71 (br. s., 2H), 5.23 (s, 2H), 7.55 (dd, 1H), 7.97 (d, 1H), 8.45 (d, 1H).

### Intermediate 30C

### 1-benzyl-3,5-dimethyl-1H-pyrazol-4-amine

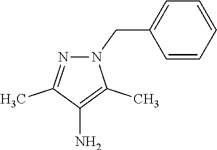

In analogy to intermediate 1C), 610 mg (2.64 mmol) 1-benzyl-3,5-dimethyl-4-nitro-1H-pyrazole (intermediate 30B) was heated with 140 mg (0.13 mmol) palladium on carbon (10 wt. %) and 1.33 g (21.12 mmol) ammonium formate in 5 mL methanol for 1 h at 80° C. to obtain 525 mg (2.61 mmol, 98%) of the desired title compound after filtration and aqueous work-up.

1H NMR (300 MHz, CDCl₃): δ (ppm)=2.06 (s, 3H), 2.21 (s, 3H), 2.43 (br. s., 2H), 5.18 (s, 2H), 7.06 (d, 2H), 7.20-7.26 (m, 1H), 7.28-7.40 (m, 2H).

### Intermediate 31C

### methyl 4-[(4-amino-3,5-dimethyl-1H-pyrazol-1-yl)methyl]benzoate

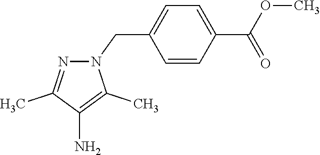

In analogy to intermediate 10C), 7.00 g (24.2 mmol) methyl 4-[(3,5-dimethyl-4-nitro-1H-pyrazol-1-yl)methyl]benzoate (intermediate 31B) were reacted to give after purification of the crude products via a Biotage chromatography system (50 g snap KP-Sil column, ethyl acetate/0-40% methanol) 5.02 g (72%) of the desired title compound, which contained small amounts of the corresponding ethyl ester.

¹H-NMR (300 MHz, DMSO d₆) δ (ppm)=1.97 (s, 3H), 1.99 (s, 3H), 3.40 (s, 2H), 3.82 (s, 3H), 5.16 (s, 2H), 7.10-7.16 (m, 2H), 7.86-7.92 (m, 2H).

### Intermediate 32C

### methyl {4-[(4-amino-3,5-dimethyl-1H-pyrazol-1-yl)methyl]phenyl}acetate

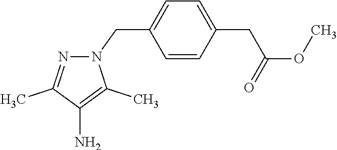

In analogy to intermediate 1C), 3.81 g (12.7 mmol) methyl {4-[(3,5-dimethyl-4-nitro-1H-pyrazol-1-yl)methyl]phenyl}acetate (intermediate 32B) were reacted to give after purification of the crude products via a Biotage chromatography system (50 g snap KP-Sil column, hexane/80-100% ethyl acetate, then ethyl acetate/0-75% methanol) 1.91 g (73%) of the desired title compound.

¹H-NMR (400 MHz, DMSO d₆) δ (ppm)=1.98 (s, 3H), 1.99 (s, 3H), 3.33 (br. s., 2H), 3.58 (s, 3H), 3.62 (s, 2H), 5.03 (s, 2H), 6.95-7.01 (m, 2H), 7.15-7.19 (m, 2H).

### Intermediate 33C

### 1-(cyclohexylmethyl)-3,5-dimethyl-1H-pyrazol-4-amine

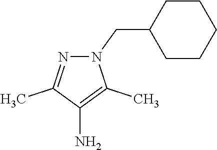

In analogy to intermediate 1C), 1.00 g (4.21 mmol) 1-(cyclohexylmethyl)-3,5-dimethyl-4-nitro-1H-pyrazole (intermediate 33B) were reacted to give after purification of the crude products via a Biotage chromatography system (25 g snap KP-Sil column, ethyl acetate/0-40% methanol) 0.68 g (78%) of the desired title compound.

¹H-NMR (300 MHz, DMSO d₆) δ (ppm)=0.80-0.98 (m, 2H), 1.06-1.20 (m, 3H), 1.47 (d, 2H), 1.54-1.71 (m, 4H), 1.95 (s, 3H), 2.02 (s, 3H), 3.24 (s, 2H), 3.61 (d, 2H).

### Intermediate 34C

### 1-[(5-chloro-2-thienyl)methyl]-3,5-dimethyl-1H-pyrazol-4-amine

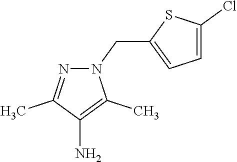

In analogy to intermediate 8C), 100 mg (0.37 mmol) 1-[(5-chloro-2-thienyl)methyl]-3,5-dimethyl-4-nitro-1H-pyrazole (intermediate 34B) were reacted to give without any further purification 150 mg (167%) of a crude product which contained the desired title compound and is used in the next step.

¹H-NMR (300 MHz, DMSO d₆) δ (ppm)=1.97 (s, 3H), 2.05 (s, 3H), 5.15 (s, 2H), 6.83 (d, 1H), 6.93 (d, 1H).

### Intermediate 35C

### 3,5-dimethyl-1-[(1-methyl-1H-pyrazol-3-yl)methyl]-1H-pyrazol-4-amine

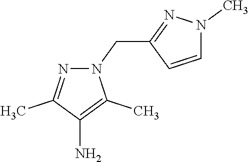

In analogy to intermediate 8C), 100 mg (0.43 mmol) 3,5-dimethyl-1-[(1-methyl-1H-pyrazol-3-yl)methyl]-4-nitro-1H-pyrazole (intermediate 35B) were reacted to give after purification of the crude products via a Biotage chromatography system (10 g snap KP-Sil column, ethyl acetate/0-100% methanol) 210 mg (241%) of a crude product which contained the desired title compound and is used in the next step.

¹H-NMR (300 MHz, DMSO d₆) δ (ppm)=1.94 (s, 3H), 2.05 (s, 3H), 3.74 (s, 3H), 4.92 (s, 2H), 5.88 (d, 1H), 7.53 (d, 2H).

### Intermediate 36C

### 3,5-dimethyl-1-[(3-methylpyridin-2-yl)methyl]-1H-pyrazol-4-amine

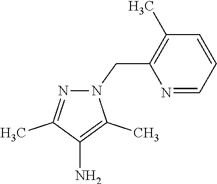 [SMILES: Cc1cccnc1Cn1nc(C)c(N)c1C]

In analogy to intermediate 1C), 339 mg (1.38 mmol) 2-[(3,5-dimethyl-4-nitro-1H-pyrazol-1-yl)methyl]-3-methylpyridine (intermediate 36B) was heated with 73 mg (0.07 mmol) palladium on carbon (10 wt. %) and 694 mg (11.01 mmol) ammonium formate in 5 mL methanol for 1 h at 80° C. to obtain 212 mg (0.98 mmol, 71%) of the desired title compound after filtration and aqueous work-up.

1H NMR (400 MHz, CDCl₃): δ (ppm)=2.07 (s, 3H), 2.22 (s, 3H), 2.42-2.67 (m, 5H), 5.27 (s, 2H), 6.46 (d, 1H), 7.01 (d, 1H), 7.46 (t, 1H).

### Intermediate 37C

### 3,5-dimethyl-1-[4-(methylsulfonyl)benzyl]-1H-pyrazol-4-amine

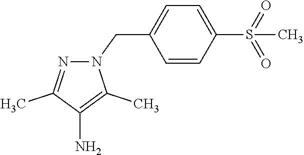

In analogy to intermediate 8C), in a first experiment 0.10 g (0.323 mmol) and in a second experiment 0.61 g (1.97 mmol) 3,5-dimethyl-1-[4-(methylsulfonyl)benzyl]-4-nitro-1H-pyrazole (intermediate 37B) were reacted to give after combined purification of both crude products via a Biotage chromatography system (25 g snap KP-Sil column, hexane/0-100% ethyl acetate, then ethyl acetate/0-25% methanol) 0.45 g (70%) of the desired title compound.

¹H-NMR (500 MHz, DMSO d₆) δ (ppm)=1.99 (s, 3H), 2.00 (s, 3H), 3.16 (s, 3H), 3.49 (s, 2H), 5.19 (s, 2H), 7.23-7.27 (m, 2H), 7.83-7.87 (m, 2H).

### Intermediate 38C

### 4-[(4-amino-3,5-dimethyl-1H-pyrazol-1-yl)methyl]-2-fluorobenzonitrile

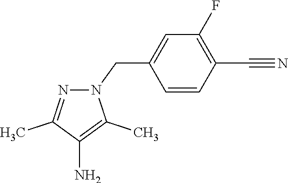 [SMILES: Cc1nn(Cc2ccc(C#N)c(F)c2)c(C)c1N]

In analogy to intermediate 8C), 1.14 g (4.16 mmol) 4-[(3,5-dimethyl-4-nitro-1H-pyrazol-1-yl)methyl]-2-fluorobenzonitrile (intermediate 38B) were reacted to give after purification of the crude product via a Biotage chromatography system (25 g snap KP-Sil column, ethyl acetate/0-40% methanol) 1.02 g (95%) of the desired title compound.

¹H-NMR (300 MHz, DMSO d₆) δ (ppm)=1.98 (s, 3H), 2.02 (s, 3H), 3.62 (br. s., 2H), 5.19 (s, 2H), 6.85 (t, 1H), 7.63 (dd, 1H), 7.85 (dd, 1H).

### Intermediate 39C

### 3-[(4-amino-3,5-dimethyl-1H-pyrazol-1-yl)methyl]pyridine-2-carbonitrile

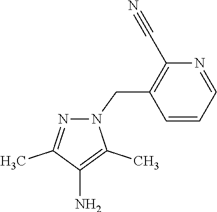 [SMILES: Cc1nn(Cc2cccnc2C#N)c(C)c1N]

In analogy to intermediate 8C), in a first experiment 0.50 g (1.94 mmol) and in a second experiment 1.86 g (7.23 mmol) 3-[(3,5-dimethyl-4-nitro-1H-pyrazol-1-yl)methyl]pyridine-2-carbonitrile (intermediate 39B) were reacted to give after combined purification of both crude products via a Biotage chromatography system (50 g snap KP-Sil column, hexane/60-100% ethyl acetate, then ethyl acetate/0-90% methanol) 0.61 g (29%) of the desired title compound.

¹H-NMR (400 MHz, DMSO d₆) δ (ppm)=1.97 (s, 3H), 2.08 (s, 3H), 3.94 (br. s., 2H), 5.28 (s, 2H), 7.36 (dd, 1H), 7.67 (dd, 1H), 8.64 (dd, 1H).

### Intermediate 40C

### 2-[(4-amino-3,5-dimethyl-1H-pyrazol-1-yl)methyl]nicotinonitrile

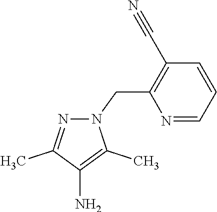 [SMILES: Cc1nn(Cc2ncccc2C#N)c(C)c1N]

In analogy to intermediate 8C), 2.16 g (8.40 mmol) 2-[(3,5-dimethyl-4-nitro-1H-pyrazol-1-yl)methyl]nicotinonitrile (intermediate 40B) were reacted to give after purification of the crude product via a Biotage chromatography system (50 g snap KP-Sil column, hexane/60-100% ethyl acetate, then ethyl acetate/0-90% methanol) 1.11 g (52%) of the desired title compound.

¹H-NMR (400 MHz, DMSO d₆) δ (ppm)=1.91 (s, 3H), 2.13 (s, 3H), 3.61 (br. s., 2H), 5.31 (s, 2H), 7.51 (dd, 1H), 8.30 (dd, 1H), 8.74 (dd, 1H).

### Intermediate 41C

### 4-[(4-amino-3,5-dimethyl-1H-pyrazol-1-yl)methyl]-N-(2-hydroxyethyl)benzamide

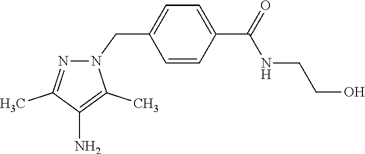 [SMILES: Cc1nn(Cc2ccc(C(=O)NCCO)cc2)c(C)c1N]

In analogy to intermediate 1C), in a first experiment of 100 mg (0.31 mmol) and in a second experiment 1.0 g (3.14 mmol) 4-[(3,5-dimethyl-4-nitro-1H-pyrazol-1-yl)methyl]-N-(2-hydroxyethyl)benzamide (intermediate 41B) were reacted to give after purification of the combined crude product via a Biotage chromatography system (25 g snap KP-Sil column, ethyl acetate/10-100% methanol) 1.04 g (104%) of the desired, not completely pure compound.

¹H-NMR (300 MHz, DMSO d₆) δ (ppm)=1.99 (s, 3H), 3.16 (s, 3H), 3.24-3.34 (m, 2H), 3.44-3.52 (m, 2H), 5.11/5.23 (s, 2H), 7.07/7.16 (d, 2H), 7.71-7.82 (m, 2H), 8.34-8.47 (m, 3H).

### Intermediate 42C

### 1-(3-fluoro-4-methoxybenzyl)-3,5-dimethyl-1H-pyrazol-4-amine

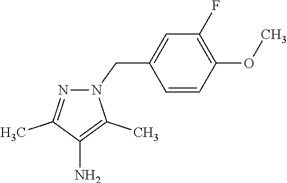

In analogy to intermediate 8C), in a first experiment of 100 mg (0.36 mmol) and in a second experiment 1.80 g (6.45 mmol) 1-(3-fluoro-4-methoxybenzyl)-3,5-dimethyl-4-nitro-1H-pyrazole (intermediate 42B) were reacted to give after purification of the combined crude product via a Biotage chromatography system (100 g snap KP-Sil column, hexane/80-100% ethyl acetate, then ethyl acetate/0-50% methanol) 1.17 g (77%) of the desired title compound.

¹H-NMR (400 MHz, DMSO d₆) δ (ppm)=1.98 (s, 3H), 1.99 (s, 3H), 3.60 (br. s., 2H), 3.78 (s, 3H), 4.99 (s, 2H), 6.79-6.89 (m, 2H), 7.07 (t, 1H).

### Intermediate 43C

### 1-[(6-methoxypyridin-3-yl)methyl]-5-methyl-3-(trifluoromethyl)-1H-pyrazol-4-amine

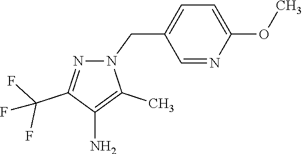 [SMILES: COc1ccc(Cn2nc(C(F)(F)F)c(N)c2C)cn1]

In analogy to intermediate 8C), 800 mg (2.53 mmol) 2-methoxy-5-{[5-methyl-4-nitro-3-(trifluoromethyl)-1H-pyrazol-1-yl]methyl}pyridine (intermediate 43B) were reacted to give after purification of the crude product via a Biotage chromatography system (25 g snap KP-Sil column, hexane/50-100% ethyl acetate, then ethyl acetate/0-50% methanol) 610 mg (79%) of the desired title compound.

¹H-NMR (400 MHz, DMSO d₆) δ (ppm)=2.11 (s, 3H), 3.81 (s, 3H), 3.98 (s, 2H), 5.19 (s, 2H), 6.79 (d, 1H), 7.46 (dd, 1H), 8.03 (d, 1H).

### Intermediate 44C

### 1-[3-(4-methoxyphenyl)propyl]-3,5-dimethyl-1H-pyrazol-4-amine

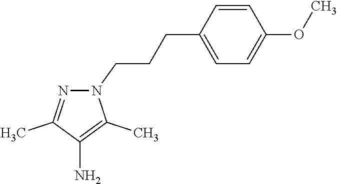

In analogy to intermediate 9C), 500 mg (1.73 mmol) 1-[3-(4-methoxyphenyl)propyl]-3,5-dimethyl-4-nitro-1H-pyrazole (intermediate 44B) were reacted to give 430 mg (93%) of a crude product of the title compound which was used without any further purification.

¹H-NMR (300 MHz, DMSO ds) δ (ppm)=1.86 (tt, 2H), 1.96 (s, 3H), 2.00 (s, 3H), 2.45 (t, 2H), 3.29 (br. s., 2H), 3.70 (s, 3H), 3.77 (t, 2H), 6.83 (d, 2H), 7.10 (d, 2H).

### Intermediate 45C

### 4-[(4-amino-3,5-dimethyl-1H-pyrazol-1-yl)methyl]nicotinonitrile

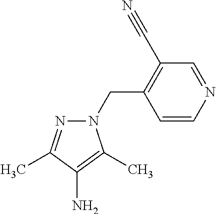 [SMILES: Cc1nn(Cc2ccncc2C#N)c(C)c1N]

In analogy to intermediate 9C), in a first experiment of 500 mg (1.94 mmol) and in a second experiment 960 mg (3.73 mmol) 4-[(3,5-dimethyl-4-nitro-1H-pyrazol-1-yl)methyl]nicotinonitrile (intermediate 45B) were reacted to give after purification of the combined crude product via a Biotage chromatography system (50 g snap KP-Sil column, ethyl acetate/0-35% methanol) 630 mg (49%) of the desired title compound.

¹H-NMR (300 MHz, DMSO d₆) δ (ppm)=1.99 (s, 3H), 2.04 (s, 3H), 3.82 (br. s., 2H), 5.30 (s, 2H), 6.75 (d, 1H), 8.73 (d, 1H), 8.99 (s, 1H).

### Intermediate 46C

### 3,5-dimethyl-1-(2-phenoxyethyl)-1H-pyrazol-4-amine

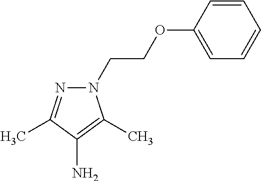 [SMILES: Cc1nn(CCOc2ccccc2)c(C)c1N]

In analogy to intermediate 9C), 1.39 g (4.86 mmol) 3,5-dimethyl-4-nitro-1-(2-phenoxyethyl)-1H-pyrazole (intermediate 46B) were reacted to give 938 mg (73%) of a crude product of the desired title compound which was used without further purification.

¹H-NMR (300 MHz, DMSO d₆) δ (ppm)=1.95 (s, 3H), 2.08 (s, 3H), 3.30 (s, 2H), 4.16-4.34 (m, 4H), 7.05 (d, 2H), 7.73 (d, 2H).

### Intermediate 47C

### 4-[(4-amino-3,5-dimethyl-1H-pyrazol-1-yl)methyl]-3-fluorobenzonitrile

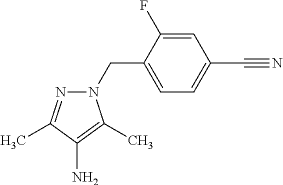 [SMILES: Cc1nn(Cc2ccc(C#N)cc2F)c(C)c1N]

In analogy to intermediate 8C), 1.84 g (6.71 mmol) 4-[(3,5-dimethyl-4-nitro-1H-pyrazol-1-yl)methyl]-3-fluorobenzonitrile (intermediate 47B) were reacted to give after purification of the crude product via a Biotage chromatography system (50 g snap KP-Sil column, hexane/25-100% ethyl acetate, then ethyl acetate/0-100% methanol) 1.24 g (72%) of the desired title compound.

¹H-NMR (400 MHz, DMSO d₆) δ (ppm)=1.98 (s, 3H), 2.02 (s, 3H), 3.47 (br. s., 2H), 5.19 (s, 2H), 6.86 (t, 1H), 7.62 (dd, 1H), 7.84 (dd, 1H).

### Intermediate 48C

### 2-{[4-amino-5-methyl-3-(trifluoromethyl)-1H-pyrazol-1-yl]methyl}benzonitrile

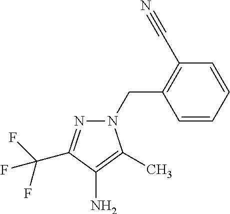 [SMILES: Cc1c(N)c(C(F)(F)F)nn1Cc1ccccc1C#N]

In analogy to intermediate 8C), 2.50 g (8.06 mmol) 2-{[5-methyl-4-nitro-3-(trifluoromethyl)-1H-pyrazol-1-yl]methyl}benzonitrile (intermediate 48B) were reacted to give after purification of the crude product via a Biotage chromatography system (50 g snap KP-Sil column, hexane/0-100% ethyl acetate, then ethyl acetate/0-35% methanol) 1.97 g (81%) of the desired title compound.

¹H-NMR (400 MHz, DMSO d₆) δ (ppm)=2.14 (s, 3H), 4.09 (s, 2H), 5.45 (s, 2H), 6.99 (d, 1H), 7.48-7.59 (m, 1H), 7.70 (td, 1H), 7.90 (dd, 1H).

### Intermediate 49C

### 1-(4-methoxybenzyl)-5-methyl-3-(trifluoromethyl)-1H-pyrazol-4-amine

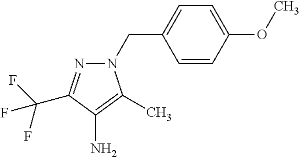 [SMILES: COc1ccc(Cn2nc(C(F)(F)F)c(N)c2C)cc1]

In analogy to intermediate 8C), in a first experiment 500 mg (1.59 mmol) and in a second experiment 6.20 g (19.7 mmol) 1-(4-methoxybenzyl)-5-methyl-4-nitro-3-(trifluoromethyl)-1H-pyrazole (intermediate 49B) were reacted to give after purification of the combined crude products via a Biotage chromatography system (100 g snap KP-Sil column, hexane/80-100% ethyl acetate, then ethyl acetate/0-50% methanol) 5.65 g (93%) of the desired title compound.

¹H-NMR (400 MHz, DMSO d₆) δ (ppm)=2.06 (s, 3H), 3.71 (s, 3H), 3.96 (br. s., 2H), 5.16 (s, 2H), 6.83-6.93 (m, 2H), 7.04-7.10 (m, 2H).

### Intermediate 50C

### 1-[(6-methoxypyridin-3-yl)methyl]-3,5-dimethyl-1H-pyrazol-4-amine

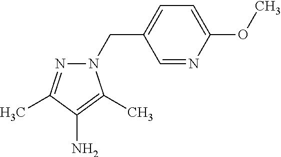

In analogy to intermediate 1C), 500 mg (1.91 mmol) 5-[(3,5-dimethyl-4-nitro-1H-pyrazol-1-yl)methyl]-2-methoxypyridine (intermediate 50B) were reacted to give after purification of the crude product via a Biotage chromatography system (10 g snap KP-Sil column, hexane/80-100% ethyl acetate, then ethyl acetate/0-75% methanol) 214 mg (46%) of the desired title compound.

¹H-NMR (400 MHz, DMSO d₆) δ (ppm)=1.97 (s, 3H), 2.02 (s, 3H), 3.35 (s, 2H), 3.80 (s, 3H), 5.00 (s, 2H), 6.74 (d, 1H), 7.38 (dd, 1H), 7.94 (d, 1H).

### Intermediate 51C

### 4-{[4-amino-5-methyl-3-(trifluoromethyl)-1H-pyrazol-1-yl]methyl}nicotinonitrile

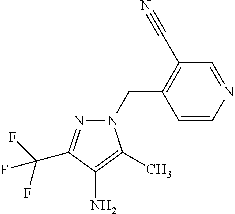 [SMILES: Cc1c(N)c(C(F)(F)F)nn1Cc1ccncc1C#N]

In analogy to intermediate 10C), 1.20 g (3.86 mmol) 4-{[5-methyl-4-nitro-3-(trifluoromethyl)-1H-pyrazol-1-yl]methyl}nicotinonitrile (intermediate 51B) were reacted to give after purification of the crude product via a Biotage chromatography system (50 g snap KP-Sil column, ethyl acetate/0-35% methanol) 690 mg (62%) of the desired title compound.

¹H-NMR (400 MHz, DMSO d₆) δ (ppm)=2.12 (s, 3H), 4.13 (s, 2H), 5.52 (s, 2H), 6.86 (d, 1H), 8.78 (d, 1H), 9.03 (s, 1H).

### Intermediate 52C

### 5-{[4-amino-5-methyl-3-(trifluoromethyl)-1H-pyrazol-1-yl]methyl}pyridine-2-carbonitrile

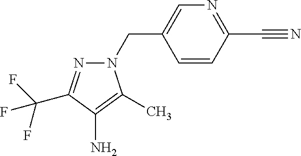 [SMILES: Cc1c(N)c(C(F)(F)F)nn1Cc1ccc(C#N)nc1]

In analogy to intermediate 8C), 2.60 g (8.25 mmol) 5-{[5-methyl-4-nitro-3-(trifluoromethyl)-1H-pyrazol-1-yl]methyl}pyridine-2-carbonitrile (intermediate 52B) were reacted to give after purification of the crude product via a Biotage chromatography system (50 g snap KP-Sil column, hexane/20-100% ethyl acetate, then ethyl acetate/0-40% methanol) 670 mg (26%) of the desired title compound.

¹H-NMR (400 MHz, DMSO d₆) δ (ppm)=2.12 (s, 3H), 4.09 (s, 2H), 5.45 (s, 2H), 7.66 (dd, 1H), 8.03 (d, 1H), 8.57 (d, 1H).

### Intermediate 53C

### 4-{[4-amino-5-methyl-3-(trifluoromethyl)-1H-pyrazol-1-yl]methyl}-3-fluorobenzonitrile

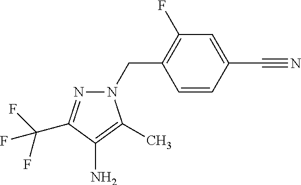 [SMILES: Cc1c(N)c(C(F)(F)F)nn1Cc1ccc(C#N)cc1F]

In analogy to intermediate 8C), in a first experiment 250 mg (0.76 mmol) and in a second experiment 1.64 g (5.00 mmol) 3-fluoro-4-{[5-methyl-4-nitro-3-(trifluoromethyl)-1H-pyrazol-1-yl]methyl}benzonitrile (intermediate 53B) were reacted to give after purification of the combined crude products via a Biotage chromatography system (50 g snap KP-Sil column, hexane/25-100% ethyl acetate, then ethyl acetate/0-100% methanol) 1.65 g (96%) of the desired title compound.

¹H-NMR (400 MHz, DMSO d₆) δ (ppm)=2.10 (s, 3H), 4.06 (s, 2H), 5.39 (s, 2H), 7.03 (t, 1H), 7.68 (dd, 1H), 7.89 (dd, 1H).

### Intermediate 54C

### 4-amino-1-(4-fluorobenzyl)-5-methyl-1H-pyrazole-3-carbonitrile

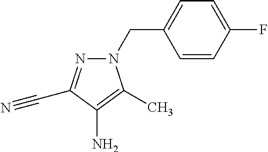 [SMILES: Cc1c(N)c(C#N)nn1Cc1ccc(F)cc1]

To a mixture of 40 ml ethanol, 20 mL water and 0.25 mL acetic acid 2.0 g (36 mmol) iron turnings was added. This mixture was heated up to 75° C. and then 1.00 g (3.84 mmol) 1-(4-fluorobenzyl)-5-methyl-4-nitro-1H-pyrazole-3-carbonitrile (intermediate 54B) was added and stirred for one hour. After cooling to 25° C. the suspension was filtered through Celite, washed with ethyl acetate and the complete filtrate was evaporated. To the residue was added 50 mL water and 40 mL of conc. aq. sodium carbonate. This aqueous phase was extracted three times with 100 mL ethyl acetate. The combined organic layer was washed with brine, dried over sodium sulfate, filtered and evaporated to obtain a crude product, which was purified via a Biotage chromatography system (50 g snap KP-Sil column, hexane/25-100% ethyl acetate then ethyl acetate/0-100% methanol) to obtain 320 mg (34%) of the desired title compound.

¹H-NMR (300 MHz, DMSO d) δ (ppm)=2.06 (s, 3H), 4.54 (s, 2H), 5.25 (s, 2H), 7.09-7.26 (m, 4H).

### Intermediate 55C

### 4-[(4-amino-3,5-diethyl-1H-pyrazol-1-yl)methyl]benzonitrile

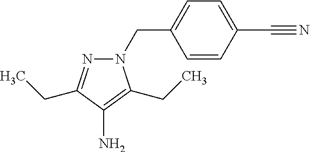 [SMILES: CCc1nn(Cc2ccc(C#N)cc2)c(CC)c1N]

In analogy to intermediate 8C), 1.29 g (4.54 mmol) 4-[(3,5-diethyl-4-nitro-1H-pyrazol-1-yl)methyl]benzonitrile (intermediate 55B) were reacted to give 1.16 g (91%) of the desired title compound, which was used without any further purification.

¹H-NMR (400 MHz, DMSO d) δ (ppm)=0.87 (t, 3H), 1.10 (t, 3H), 2.40-2.47 (m, 4H), 3.42 (s, 2H), 5.21 (s, 2H), 7.14 (d, 2H), 7.76 (d, 2H).

### Intermediate 56C

### 4-{[4-amino-5-ethyl-3-(trifluoromethyl)-1H-pyrazol-1-yl]methyl}benzonitrile

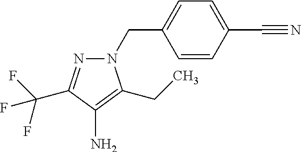 [SMILES: CCc1c(N)c(C(F)(F)F)nn1Cc1ccc(C#N)cc1]

In analogy to intermediate 8C), in a first experiment 200 mg (0.62 mmol) and in a second experiment 1.00 g (3.08 mmol) 4-{[5-ethyl-4-nitro-3-(trifluoromethyl)-1H-pyrazol-1-yl]methyl}benzonitrile (intermediate 56B) were reacted to give after purification of the combined crude products via a Biotage chromatography system (25 g snap KP-Sil column, hexane/50-100% ethyl acetate, then ethyl acetate/0-30% methanol) 1.02 g (94%) of the desired title compound.

¹H-NMR (400 MHz, DMSO d₆) δ (ppm)=0.87 (t, 3H), 2.55 (q, 2H), 4.05 (s, 2H), 5.39 (s, 2H), 7.23 (d, 2H), 7.82 (d, 2H).

### Intermediate 57C

### 4-{[4-amino-5-isopropyl-3-(trifluoromethyl)-1H-pyrazol-1-yl]methyl}benzonitrile

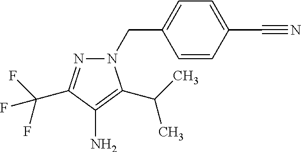 [SMILES: CC(C)c1c(N)c(C(F)(F)F)nn1Cc1ccc(C#N)cc1]

In analogy to intermediate 8C), 400 mg (1.18 mmol) 4-{[5-isopropyl-4-nitro-3-(trifluoromethyl)-1H-pyrazol-1-yl]methyl}benzonitrile (intermediate 57B) were reacted to give 380 mg (103%, purity about 80%) of the desired title compound, which was used without any further purification.

¹H-NMR (400 MHz, DMSO d₆) δ (ppm)=1.11 (d, 6H), 3.04 (spt, 1H), 3.90 (s, 2H), 5.45 (s, 2H), 7.20 (d, 2H), 7.84 (d, 2H).

### Intermediate 58C

### 4-{[4-amino-3-isopropyl-5-(trifluoromethyl)-1H-pyrazol-1-yl]methyl}benzonitrile

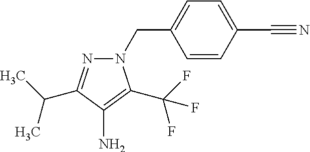 [SMILES: CC(C)c1nn(Cc2ccc(C#N)cc2)c(C(F)(F)F)c1N]

In analogy to intermediate 8C), 440 mg (1.30 mmol) 4-{[3-isopropyl-4-nitro-5-(trifluoromethyl)-1H-pyrazol-1-yl]methyl}benzonitrile (intermediate 58B) were reacted to give after purification of the crude product via a Biotage chromatography system (25 g snap KP-Sil column, hexane/0-100% ethyl acetate, then ethyl acetate/0-50% methanol) 350 mg (85%) of the desired title compound.

¹H-NMR (300 MHz, DMSO d₆) δ (ppm)=1.17 (d, 6H), 3.00 (spt, 1H), 4.44 (s, 2H), 5.33 (s, 2H), 7.13 (d, 2H), 7.78 (d, 2H).

### Intermediate 59C and 60C

### 4-[(4-amino-3-isopropyl-5-methyl-1H-pyrazol-1-yl)methyl]benzonitrile and 4-[(4-amino-5-isopropyl-3-methyl-1H-pyrazol-1-yl)methyl]benzonitrile

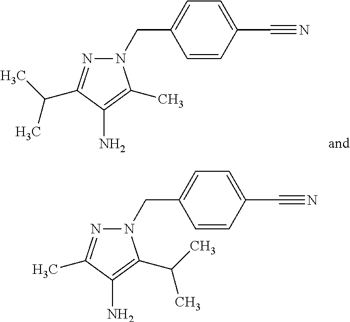 [SMILES: Cc1c(N)c(C(C)C)nn1Cc1ccc(C#N)cc1 ; Cc1nn(Cc2ccc(C#N)cc2)c(C(C)C)c1N]

In analogy to intermediate 8C), in a first experiment 1.00 (3.52 mmol) and in a second experiment 2.22 g (7.81 mmol) 4-[(3-isopropyl-5-methyl-4-nitro-1H-pyrazol-1-yl)methyl]benzonitrile (intermediate 59B) were reacted to give after purification of the combined crude products via a Biotage chromatography system (50 g snap KP-Sil column, hexane/0-100% ethyl acetate, then ethyl acetate/0-80% methanol) 2.32 g (48%) 4-[(4-amino-3-isopropyl-5-methyl-1H-pyrazol-1-yl)methyl]benzonitrile as the main product together with a small amount of its regioisomer 4-[(4-amino-5-isopropyl-3-methyl-1H-pyrazol-1-yl)methyl]benzonitrile as desired title compounds.

NMR of 4-[(4-amino-3-isopropyl-5-methyl-1H-pyrazol-1-yl)methyl]benzonitrile as the main product:

¹H-NMR (300 MHz, DMSO d₆) δ (ppm)=1.15 (d, 6H), 1.95 (s, 3H), 2.89 (spt, 1H), 3.76 (s, 2H), 5.20 (s, 2H), 7.12 (d, 2H), 7.76 (d, 2H).

### Intermediate 61C and 62C

### 4-[(4-amino-3-ethyl-5-methyl-1H-pyrazol-1-yl)methyl]benzonitrile and 4-[(4-amino-5-ethyl-3-methyl-1H-pyrazol-1-yl)methyl]benzonitrile

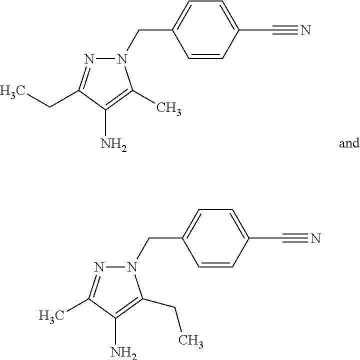 [SMILES: CCc1nn(Cc2ccc(C#N)cc2)c(C)c1N ; CCc1c(N)c(C)nn1Cc1ccc(C#N)cc1]

In analogy to intermediate 8C), 650 mg (2.41 mmol) of the mixture of 4-[(3-ethyl-5-methyl-4-nitro-1H-pyrazol-1-yl)methyl]benzonitrile and 4-[(5-ethyl-3-methyl-4-nitro-1H-pyrazol-1-yl)methyl]benzonitrile (intermediate 61 and 62B) were reacted to give 571 mg (96%) of a mixture 4-[(4-amino-3-ethyl-5-methyl-1H-pyrazol-1-yl)methyl]benzonitrile and 4-[(4-amino-5-ethyl-3-methyl-1H-pyrazol-1-yl)methyl]benzonitrile as desired title compounds, which were used without any further purification.

NMR of the mixture:

¹H-NMR (400 MHz, DMSO d₆) δ (ppm)=0.89/1.11 (t, 3H), 1.98/2.01 (s, 3H), 2.40-2.49 (m, 2H), 5.21 (s, 2H), 7.12-7.20 (m, 2H), 7.76-7.82 (m, 2H).

### Intermediate 63C

### (±)-4-[1-(4-amino-3,5-dimethyl-1H-pyrazol-1-yl)ethyl]benzonitrile

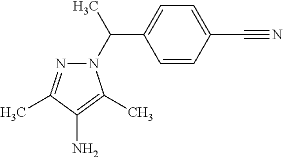 [SMILES: Cc1nn(C(C)c2ccc(C#N)cc2)c(C)c1N]

In analogy to intermediate 8C), 440 mg (1.63 mmol) of (±)-4-[1-(3,5-dimethyl-4-nitro-1H-pyrazol-1-yl)ethyl]benzonitrile (intermediate 63B) were reacted to give 410 mg (91%) of the desired title compound, which was used without any further purification.

¹H-NMR (400 MHz, DMSO d₆) δ (ppm)=1.69 (d, 3H), 1.93 (s, 3H), 2.02 (s, 3H), 3.41 (br. s., 2H), 5.44 (q, 1H), 7.21 (d, 2H), 7.71-7.77 (m, 2H).

### Intermediate 64C

### (±)-4-{1-[4-amino-5-methyl-3-(trifluoromethyl)-1H-pyrazol-1-yl]ethyl}benzonitrile

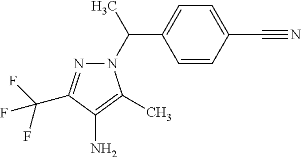 [SMILES: Cc1c(N)c(C(F)(F)F)nn1C(C)c1ccc(C#N)cc1]

In analogy to intermediate 8C), 1.54 g (4.75 mmol) of (±)-4-{1-[5-methyl-4-nitro-3-(trifluoromethyl)-1H-pyrazol-1-yl]ethyl}benzonitrile (intermediate 64B) were reacted to give after purification of the crude product via a Biotage chromatography system (25 g snap KP-Sil column, hexane/30-100% ethyl acetate) 1.03 g (66%) of the desired title compound.

¹H-NMR (400 MHz, DMSO d₆) δ (ppm)=1.74 (d, 3H), 2.02 (s, 3H), 4.03 (s, 2H), 5.70 (q, 1H), 7.28 (d, 2H), 7.80-7.84 (m, 2H).

### Intermediate 65C

### (±)-5-methyl-1-(1-phenylethyl)-3-(trifluoromethyl)-1H-pyrazol-4-amine

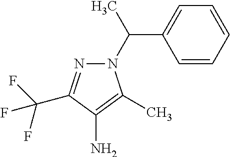 [SMILES: Cc1c(N)c(C(F)(F)F)nn1C(C)c1ccccc1]

In analogy to intermediate 8C), in a first experiment 200 mg (0.67 mmol) and in a second experiment 800 mg (2.67 mmol) of (±)-5-methyl-4-nitro-1-(1-phenylethyl)-3-(trifluoromethyl)-1H-pyrazole (intermediate 65B) were reacted to give after two subsequent purifications of the combined crude products via a Biotage chromatography system (both 50 g snap KP-Sil column, hexane/0-100% ethyl acetate, then ethyl acetate/0-100% methanol) 760 mg (95%) of the desired title compound.

¹H-NMR (400 MHz, DMSO d₆) δ (ppm)=1.72 (d, 3H), 2.01 (s, 3H), 3.94 (s, 2H), 5.55 (q, 1H), 7.07-7.12 (m, 2H), 7.19-7.38 (m, 3H).

### Intermediate 66C

### 4-[3-(4-amino-3,5-dimethyl-1H-pyrazol-1-yl)propyl]benzonitrile

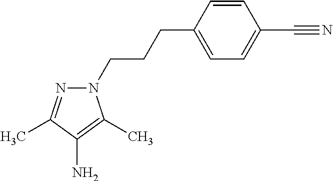 [SMILES: Cc1nn(CCCc2ccc(C#N)cc2)c(C)c1N]

In analogy to intermediate 10C), 1.05 g (3.67 mmol) of 34-[3-(3,5-dimethyl-4-nitro-1H-pyrazol-1-yl)propyl]benzonitrile (intermediate 66B) were reacted to give 931 mg (86%) of the desired title compound, which was used without any further purification.

¹H-NMR (300 MHz, DMSO d₆) δ (ppm)=1.86-1.97 (m, 5H), 2.01 (s, 3H), 2.61 (t, 2H), 3.36 (br. s., 2H), 3.80 (t, 2H), 7.41 (d, 2H), 7.73 (d, 2H).

### Intermediate 67C

### 6-[(4-amino-3,5-dimethyl-1H-pyrazol-1-yl)methyl]nicotinonitrile

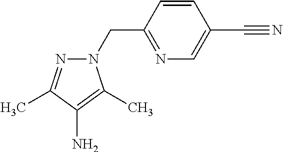 [SMILES: Cc1nn(Cc2ccc(C#N)cn2)c(C)c1N]

In analogy to intermediate 8C), 2.11 g (8.20 mmol) of 6-[(3,5-dimethyl-4-nitro-1H-pyrazol-1-yl)methyl]nicotinonitrile (intermediate 67B) were reacted to give after purification of the crude product via a Biotage chromatography system (25 g snap KP-Sil column, hexane/25-100% ethyl acetate, then ethyl acetate/0-100% methanol) 760 mg (44%) of the desired title compound.

¹H-NMR (400 MHz, DMSO d₆) δ (ppm)=2.00 (s, 3H), 2.03 (s, 3H), 3.65 (br. s., 2H), 5.26 (s, 2H), 6.89 (d, 1H), 8.24 (dd, 1H), 8.97 (d, 1H).

### Intermediate 68C

### 6-{[4-amino-5-methyl-3-(trifluoromethyl)-1H-pyrazol-1-yl]methyl}nicotinonitrile

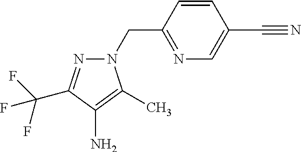 [SMILES: Cc1c(N)c(C(F)(F)F)nn1Cc1ccc(C#N)cn1]

In analogy to intermediate 8C), 2.15 g (8.07 mmol) of 6-{[5-methyl-4-nitro-3-(trifluoromethyl)-1H-pyrazol-1-yl]methyl}nicotinonitrile (intermediate 68B) were reacted to give after purification of the crude product via a Biotage chromatography system (25 g snap KP-Sil column, hexane/25-100% ethyl acetate, then ethyl acetate/0-100% methanol) 1.94 g (77%) of the desired title compound.

¹H-NMR (400 MHz, DMSO d) δ (ppm)=2.11 (s, 3H), 4.07 (s, 2H), 5.48 (s, 2H), 7.20 (d, 1H), 8.31 (dd, 1H), 8.99 (d, 1H).

### Intermediate 69C

### methyl 4-{[4-amino-5-methyl-3-(trifluoromethyl)-1H-pyrazol-1-yl]methyl}benzoate

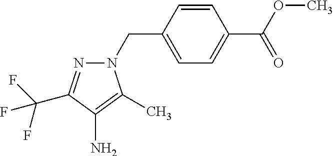 [SMILES: COC(=O)c1ccc(Cn2nc(C(F)(F)F)c(N)c2C)cc1]

In analogy to intermediate 10C), 3.00 g (8.74 mmol) of methyl 4-{[5-methyl-4-nitro-3-(trifluoromethyl)-1H-pyrazol-1-yl]methyl}benzoate (intermediate 69B) were reacted to give after purification of the crude product via a Biotage chromatography system (100 g snap KP-Sil column, hexane/0-100% ethyl acetate) 2.62 g (86%) of the desired title compound.

¹H-NMR (300 MHz, DMSO d₆) δ (ppm)=2.05 (s, 3H), 3.83 (s, 3H), 4.05 (s, 2H), 5.36 (s, 2H), 7.20 (d, 2H), 7.93 (d, 2H).

### Intermediate 70C

### (±)-ethyl [{4-[(4-amino-3,5-dimethyl-1H-pyrazol-1-yl)methyl]phenyl}(methyl)oxido-λ⁶-sulfanylidene]carbamate

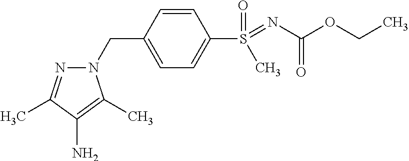 [SMILES: CCOC(=O)N=S(C)(=O)c1ccc(Cn2nc(C)c(N)c2C)cc1]

In analogy to intermediate 8C), in a first experiment 500 mg (1.31 mmol) and in a second experiment 1.60 g (4.21 mmol) of (±)-ethyl [{4-[(3,5-dimethyl-4-nitro-1H-pyrazol-1-yl)methyl]phenyl}(methyl)oxido-λ⁶-sulfanylidene]carbamate (intermediate 70B) were reacted to give after purification of the combined crude products via a Biotage chromatography system (100 g snap KP-Sil column, hexane/0-100% ethyl acetate, then ethyl acetate/0-25% methanol) 650 mg (34%) of the desired title compound.

¹H-NMR (400 MHz, DMSO d₆) δ (ppm)=1.09 (t, 3H), 2.02 (s, 3H), 2.03 (s, 3H), 3.18 (s, 2H), 3.43 (s, 3H), 3.86-3.96 (m, 2H), 5.22 (s, 2H), 7.30 (d, 2H), 7.87-7.91 (m, 2H).

### Intermediate 71C

### 1-{4-[(dimethylamino)methyl]benzyl}-3,5-dimethyl-1H-pyrazol-4-amine

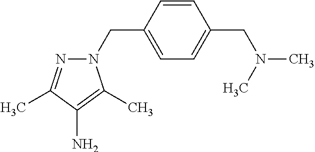

In analogy to intermediate 10C), 100 mg (0.35 mmol) of 1-{4-[(3,5-dimethyl-4-nitro-1H-pyrazol-1-yl)methyl]phenyl}-N,N-dimethylmethanamine (intermediate 71B) were reacted to give 79 mg (83%) of the desired title compound, which was use without any further purification.

¹H-NMR (300 MHz, DMSO d₆) δ (ppm)=1.98 (s, 6H), 2.09 (s, 6H), 3.42 (br. s., 2H), 5.04 (s, 2H), 6.98 (d, 2H), 7.19 (d, 2H).

### Intermediate 72C

### 4-{[4-amino-3-methyl-5-(trifluoromethyl)-1H-pyrazol-1-yl]methyl}benzonitrile

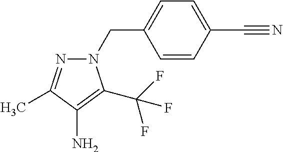 [SMILES: Cc1nn(Cc2ccc(C#N)cc2)c(C(F)(F)F)c1N]

In analogy to intermediate 8C), in a first experiment 150 mg (0.48 mmoL) and in a second experiment 540 mg (8.26 mmol) of 4-{[3-methyl-4-nitro-5-(trifluoromethyl)-1H-pyrazol-1-yl]methyl}benzonitrile (intermediate 72B) were reacted to give after purification of the combined crude products via a Biotage chromatography system (25 g snap KP-Sil column, hexane/20-70% ethyl acetate) 536 mg (86%) of the desired title compound.

¹H-NMR (400 MHz, DMSO d₆) δ (ppm)=2.09 (s, 3H), 4.49 (s, 2H), 5.32 (s, 2H), 7.19 (d, 2H), 7.77-7.84 (m, 2H).

### Intermediate 73C

### 4-[(4-amino-3,5-dimethyl-1H-pyrazol-1-yl)methyl]-3,5-difluorobenzonitrile

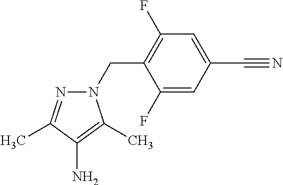 [SMILES: Cc1nn(Cc2c(F)cc(C#N)cc2F)c(C)c1N]

In analogy to intermediate 8C), 1.03 g (3.52 mmol) of 4-[(3,5-dimethyl-4-nitro-1H-pyrazol-1-yl)methyl]-3,5-difluorobenzonitrile (intermediate 73B) were reacted to give after purification of the crude product via a Biotage chromatography system (25 g snap KP-Sil column, hexane/50-100% ethyl acetate, then ethyl acetate/0-100% methanol) 620 mg (62%) of the desired title compound.

¹H-NMR (400 MHz, DMSO d₆) δ (ppm)=1.90 (s, 3H), 2.15 (s, 3H), 3.75 (s, 2H), 5.11 (s, 2H), 7.76-7.86 (m, 2H).

### Intermediate 74C

### 4-{[4-amino-5-methyl-3-(trifluoromethyl)-1H-pyrazol-1-yl]methyl}-3,5-difluorobenzonitrile

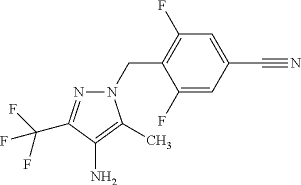 [SMILES: Cc1c(N)c(C(F)(F)F)nn1Cc1c(F)cc(C#N)cc1F]

In analogy to intermediate 8C), 1.90 g (4.67 mmol) of 3,5-difluoro-4-{[5-methyl-4-nitro-3-(trifluoromethyl)-1H-pyrazol-1-yl]methyl}benzonitrile (intermediate 74B) were reacted to give after purification of the crude product via a Biotage chromatography system (25 g snap KP-Sil column, hexane/50-100% ethyl acetate, then ethyl acetate/0-100% methanol) 1.46 g (79%) of the desired title compound.

¹H-NMR (400 MHz, DMSO d₆) δ (ppm)=2.20 (s, 3H), 3.99 (s, 2H), 5.31 (s, 2H), 7.80-7.87 (m, 2H).

### Intermediate 75C

### 1-[4-(methoxymethyl)benzyl]-5-methyl-3-(trifluoromethyl)-1H-pyrazol-4-amine

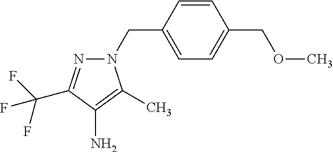 [SMILES: COCc1ccc(Cn2nc(C(F)(F)F)c(N)c2C)cc1]

In analogy to intermediate 1C), 380 mg (1.15 mmol) of 1-[4-(methoxymethyl)benzyl]-5-methyl-4-nitro-3-(trifluoromethyl)-1H-pyrazole (intermediate 75B) were reacted to give after purification of the crude product via a Biotage chromatography system (10 g snap KP-Sil column, hexane/0-100% ethyl acetate, then ethyl acetate/0-100% methanol) 280 mg (77%) of the desired title compound.

¹H-NMR (400 MHz, DMSO d₆) δ (ppm)=2.07 (s, 3H), 3.27 (s, 3H), 4.01 (s, 2H), 4.38 (s, 2H), 5.26 (s, 2H), 7.09 (d, 2H), 7.29 (d, 2H).

### Intermediate 76C

### (4-{[4-amino-5-methyl-3-(trifluoromethyl)-1H-pyrazol-1-yl]methyl}phenyl)acetonitrile

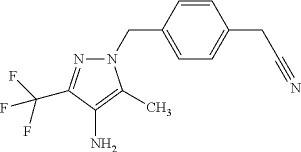 [SMILES: Cc1c(N)c(C(F)(F)F)nn1Cc1ccc(CC#N)cc1]

In analogy to intermediate 8C), 780 mg (2.41 mmol) of (4-{[5-methyl-4-nitro-3-(trifluoromethyl)-1H-pyrazol-1-yl]methyl}phenyl)acetonitrile (intermediate 76B) were reacted to give after purification of the crude product via a Biotage chromatography system (25 g snap KP-Sil column, hexane/0-100% ethyl acetate, then ethyl acetate/0-100% methanol) 580 mg (78%) of the desired title compound.

¹H-NMR (400 MHz, DMSO d₆) δ (ppm)=2.08 (s, 3H), 3.99-4.03 (m, 4H), 5.26 (s, 2H), 7.13 (d, 2H), 7.33 (d, 2H).

### Intermediate 77C

### 3-[(4-amino-3,5-dimethyl-1H-pyrazol-1-yl)methyl]-N-methyl-1,2,4-oxadiazole-5-carboxamide

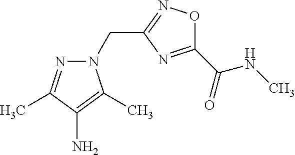

In analogy to intermediate 10C), 621 mg (2.11 mmol) 3-[(3,5-dimethyl-4-nitro-1H-pyrazol-1-yl)methyl]-N-methyl-1,2,4-oxadiazole-5-carboxamide (intermediate 77B) were reacted to give 380 mg (58%) of the desired title compound without purification.

¹H-NMR (400 MHz, CDCl₃) δ (ppm)=2.17 (s, 3H), 2.20 (s, 3H), 2.55 (br. s., 2H), 3.01 (d, 3H), 5.30 (s, 2H), 7.09 (br. s., 1H).

### Intermediate 78C

### tert-butyl 4-[(4-amino-3,5-dimethyl-1H-pyrazol-1-yl)methyl]piperidine-1-carboxylate

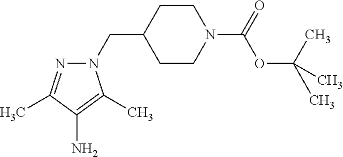 [SMILES: Cc1nn(CC2CCN(C(=O)OC(C)(C)C)CC2)c(C)c1N]

In analogy to intermediate 1C), 3.54 g (8.37 mmol) tert-butyl 4-[(3,5-dimethyl-4-nitro-1H-pyrazol-1-yl)methyl]piperidine-1-carboxylate (intermediate 78B) were reacted to give 1.68 g (62%) of the desired title compound after flash chromatography.

¹H NMR (300 MHz, CDCl₃): δ (ppm)=1.14 (dddd, 2H), 1.44 (s, 9H), 1.54 (br. d., 2H), 2.01 (m, 1H), 2.16 (s, 3H), 2.18 (s, 3H), 2.64 (dd, 2H), 3.77 (d, 2H), 4.09 (m, 2H).

### Intermediate 79C

### 5-methyl-1-[(5-methyl-1,2-oxazol-3-yl)methyl]-3-(trifluoromethyl)-1H-pyrazol-4-amine

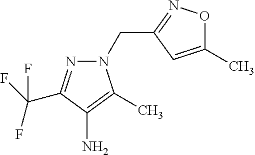 [SMILES: Cc1cc(Cn2nc(C(F)(F)F)c(N)c2C)no1]

In analogy to intermediate 10C), 4.55 g (15.7 mmol) 5-methyl-3-{[5-methyl-4-nitro-3-(trifluoromethyl)-1H-pyrazol-1-yl]methyl}-1,2-oxazole (intermediate 79B) were reacted to give 3.08 g (68%) of the desired title compound without purification.

¹H NMR (400 MHz, DMSO-d₆): δ (ppm)=2.14 (s, 3H), 2.36 (s, 3H), 4.02 (br. s., 2H), 5.29 (s, 2H), 6.03 (s, 1H).

### Intermediate 80C

### 1-[(5-ethyl-1,2,4-oxadiazol-3-yl)methyl]-5-methyl-3-(trifluoromethyl)-1H-pyrazol-4-amine

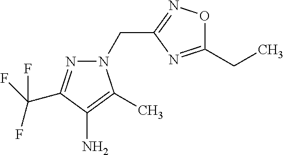 [SMILES: CCc1nc(Cn2nc(C(F)(F)F)c(N)c2C)no1]

In analogy to intermediate 10C), 1.94 g (6.04 mmol) 5-methyl-3-{[5-methyl-4-nitro-3-(trifluoromethyl)-1H-pyrazol-1-yl]methyl}-1,2-oxazole (intermediate 80B) were reacted to give 1.49 g (81%) of the desired title compound without purification.

¹H NMR (400 MHz, DMSO-d₆): δ (ppm)=1.25 (t, 3H), 2.18 (s, 3H), 2.93 (q, 2H), 4.04 (br. s., 2H), 5.42 (s, 2H).

### Intermediate 81C

### 1-[(3-ethyl-1,2-oxazol-5-yl)methyl]-5-methyl-3-(trifluoromethyl)-1H-pyrazol-4-amine

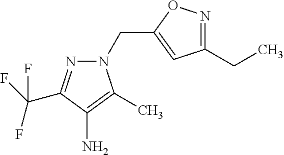 [SMILES: CCc1cc(Cn2nc(C(F)(F)F)c(N)c2C)on1]

In analogy to intermediate 10C), 690 mg (2.29 mmol) 3-ethyl-5-{[5-methyl-4-nitro-3-(trifluoromethyl)-1H-pyrazol-1-yl]methyl}-1,2-oxazole (intermediate 81B) were reacted to give 269 mg (41%) of the desired title compound without purification.

¹H NMR (400 MHz, DMSO-d₆): δ (ppm)=1.16 (t, 3H), 2.18 (s, 3H), 2.59 (q, 2H), 4.06 (br. s., 2H), 5.43 (s, 2H), 6.32 (s, 1H).

### Intermediate 82C

### 3,5-dimethyl-1-[(3-methyl-1,2-oxazol-5-yl)methyl]-1H-pyrazol-4-amine

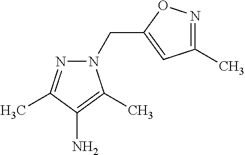

In analogy to intermediate 10C), 630 mg (2.67 mmol) 5-[(3,5-dimethyl-4-nitro-1H-pyrazol-1-yl)methyl]-3-methyl-1,2-oxazole (intermediate 82B) were reacted to give 442 mg (76%) of the desired title compound without purification.

¹H NMR (400 MHz, DMSO-d₆): δ (ppm)=1.97 (s, 3H), 2.09 (s, 3H), 2.17 (s, 3H), 3.62 (br. s., 2H), 5.19 (s, 2H), 6.09 (s, 1H).

### Intermediate 83C

### 3,5-dimethyl-1-[(5-methyl-1,2-oxazol-3-yl)methyl]-1H-pyrazol-4-amine

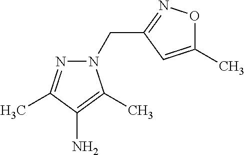

In analogy to intermediate 8C), 200 mg (847 μmol) 3-[(3,5-dimethyl-4-nitro-1H-pyrazol-1-yl)methyl]-5-methyl-1,2-oxazole (intermediate 83B) were reacted to give 67 mg (31%) of the desired title compound without purification.

¹H NMR (400 MHz, DMSO-d₆): δ (ppm)=1.98 (s, 3H), 2.06 (s, 3H), 2.33 (s, 3H), 3.97 (br. s., 2H), 5.07 (s, 2H), 5.90 (s, 1H).

### Intermediate 84C

### tert-butyl 4-{[4-amino-5-methyl-3-(trifluoromethyl)-1H-pyrazol-1-yl]methyl}piperidine-1-carboxylate

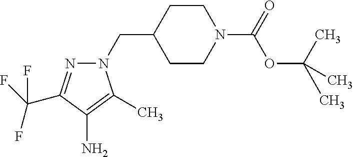 [SMILES: Cc1c(N)c(C(F)(F)F)nn1CC1CCN(C(=O)OC(C)(C)C)CC1]

In analogy to intermediate 1C), 1.89 g (4.58 mmol) tert-butyl 4-{[5-methyl-4-nitro-3-(trifluoromethyl)-1H-pyrazol-1-yl]methyl}piperidine-1-carboxylate (intermediate 84B) were reacted to give 1.62 g (59%) of the desired title compound which was used without further purification.

¹H NMR (400 MHz, CDCl₃): δ (ppm)=1.17 (dddd, 2H), 1.46 (s, 9H), 1.54 (m, 2H), 2.07 (m, 1H), 2.17 (s, 3H), 2.66 (m, 2H), 3.87 (d, 2H), 4.12 (m, 2H).

### Intermediate 85C

### 5-methyl-1-(piperidin-4-ylmethyl)-3-(trifluoromethyl)-1H-pyrazol-4-amine

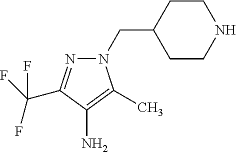 [SMILES: Cc1c(N)c(C(F)(F)F)nn1CC1CCNCC1]

A solution of 800 mg (1.88 mmol) tert-butyl 4-{[4-amino-5-methyl-3-(trifluoromethyl)-1H-pyrazol-1-yl]methyl}piperidine-1-carboxylate (intermediate 84C) in 11 mL dichloromethane was stirred with 1.45 mL (18.8 mmol) trifluoroacetic acid for 4 hours. The reaction mixture was filtered over NH₂ derivatized silica gel, and the filtrate was evaporated yielding 1.0 g of the desired title compound as crude product which was used without further purification.

¹H NMR (300 MHz, DMSO-d₆): δ (ppm)=1.33 (dddd, 2H), 1.62 (m, 2H), 2.08 (m, 1H), 2.18 (s, 3H), 2.83 (m, 2H), 3.25 (m, 2H), 3.94 (d, 2H).

### Intermediate 86C

### 1-{[1-(ethylsulfonyl)piperidin-4-yl]methyl}-5-methyl-3-(trifluoromethyl)-1H-pyrazol-4-amine

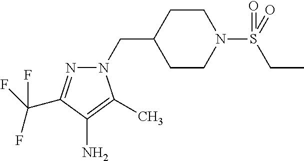 [SMILES: CCS(=O)(=O)N1CCC(Cn2nc(C(F)(F)F)c(N)c2C)CC1]

A solution of 500 mg (crude,−800 μmol) 5-methyl-1-(piperidin-4-ylmethyl)-3-(trifluoromethyl)-1H-pyrazol-4-amine (intermediate 85C) in 3 mL DMF was stirred with 106 μL (1.12 mmol) ethanesulfonyl chloride and 670 μL (4.80 mmol) triethylamine for 30 minutes. Saturated aqueous sodium bicarbonate and ethyl acetate were added to the reaction. The mixture was extracted with butanol, and the combined organic phase was washed with brine, dried, filtered, and evaporated. Purification by flash chromatography yielded 144 mg (48%) of the desired title compound.

¹H NMR (400 MHz, DMSO-d₆): δ (ppm)=1.19 (t, 3H), 1.21 (dddd, 2H), 1.53 (m, 2H), 1.91 (m, 1H), 2.14 (s, 3H), 2.75 (m, 2H), 3.00 (q, 2H), 3.58 (m, 2H), 3.90 (d, 2H), 3.94 (s, 2H).

### Intermediate 87C

### 1-[(3-ethyl-1,2-oxazol-5-yl)methyl]-3,5-dimethyl-1H-pyrazol-4-amine

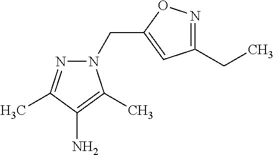 [SMILES: CCc1cc(Cn2nc(C)c(N)c2C)on1]

In analogy to intermediate 10C), 1.34 g (4.82 mmol) 5-[(3,5-dimethyl-4-nitro-1H-pyrazol-1-yl)methyl]-3-ethyl-1,2-oxazole (intermediate 85B) were reacted to give 1.09 g (95%) of the desired title compound without purification.

¹H NMR (400 MHz, DMSO-d₆): δ (ppm)=1.14 (t, 3H), 1.96 (s, 3H), 2.09 (s, 3H), 2.57 (q, 2H), 3.41 (br. s., 2H), 5.19 (s, 2H), 6.15 (s, 1H).

### Intermediate 88C

### 1-{[1-(ethylsulfonyl)piperidin-4-yl]methyl}-3,5-dimethyl-1H-pyrazol-4-amine

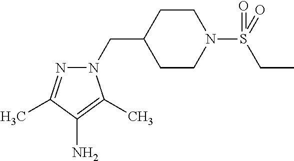 [SMILES: CCS(=O)(=O)N1CCC(Cn2nc(C)c(N)c2C)CC1]

In analogy to intermediate 10C), 2.53 g (7.27 mmol) 4-[(3,5-dimethyl-4-nitro-1H-pyrazol-1-yl)methyl]-1-(ethylsulfonyl)piperidine (intermediate 87B) were reacted to give 2.20 g (96%) of the desired title compound after flash chromatography.

¹H NMR (400 MHz, DMSO-d₆): δ (ppm)=1.17 (m, 2H), 1.19 (t, 3H), 1.52 (m, 2H), 1.84 (m, 1H), 1.96 (s, 3H), 2.05 (s, 3H), 2.72 (m, 2H), 2.99 (q, 2H), 3.27 (s, 2H), 3.56 (m, 2H), 3.71 (d, 2H).

### Intermediate 89C

### 3,5-dimethyl-1-{[3-(propan-2-yl)-1,2-oxazol-5-yl]methyl}-1H-pyrazol-4-amine

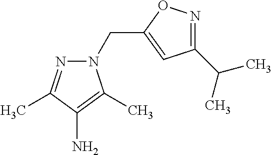 [SMILES: Cc1nn(Cc2cc(C(C)C)no2)c(C)c1N]

In analogy to intermediate 10C), 1.06 g (3.61 mmol) 5-[(3,5-dimethyl-4-nitro-1H-pyrazol-1-yl)methyl]-3-(propan-2-yl)-1,2-oxazole (intermediate 88B) were reacted to give 877 mg (99%) of the desired title compound without purification.

¹H NMR (400 MHz, DMSO-d₆): δ (ppm)=1.17 (d, 6H), 1.97 (s, 3H), 2.10 (s, 3H), 2.94 (sept, 1H), 3.41 (br. s., 2H), 5.19 (s, 2H), 6.21 (s, 1H).

### Intermediate 90C

### 1-[(5-cyclopropyl-1,2-oxazol-3-yl)methyl]-3,5-dimethyl-1H-pyrazol-4-amine

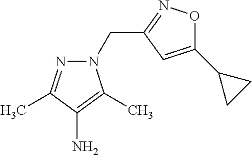 [SMILES: Cc1nn(Cc2cc(C3CC3)on2)c(C)c1N]

In analogy to intermediate 10C), 1.30 g (3.77 mol) 5-cyclopropyl-3-[(3,5-dimethyl-4-nitro-1H-pyrazol-1-yl)methyl]-1,2-oxazole (intermediate 89B) were reacted to give 1.07 g (98%) of the desired title compound without purification.

¹H NMR (300 MHz, DMSO-d₆): δ (ppm)=0.84 (m, 2H), 1.01 (m, 2H), 1.97 (s, 3H), 2.05 (s, 3H), 2.08 (m, 1H), 3.39 (br. s., 2H), 5.03 (s, 2H), 5.88 (s, 1H).

### Intermediate 91C

### 5-methyl-1-[(3-methyl-1,2-oxazol-5-yl)methyl]-3-(trifluoromethyl)-1H-pyrazol-4-amine

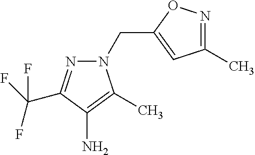 [SMILES: Cc1cc(Cn2nc(C(F)(F)F)c(N)c2C)on1]

In analogy to intermediate 10C), 1.40 g (4.58 mmol) 3-methyl-5-{[5-methyl-4-nitro-3-(trifluoromethyl)-1H-pyrazol-1-yl]methyl}-1,2-oxazole (intermediate 90B) were reacted to give 1.00 g (75%) of the desired title compound without purification.

¹H NMR (400 MHz, DMSO-d₆): δ (ppm)=2.17 (s, 3H), 2.20 (s, 3H), 4.04 (br. s., 2H), 5.43 (s, 2H), 6.25 (s, 1H).

### Intermediate 92C

### 5-[(4-amino-3,5-dimethyl-1H-pyrazol-1-yl)methyl]thiophene-2-carbonitrile

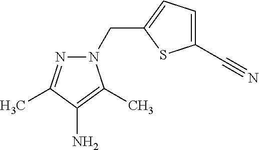 [SMILES: Cc1nn(Cc2ccc(C#N)s2)c(C)c1N]

In analogy to intermediate 8C), 468 mg (1.43 mmol) 5-[(3,5-dimethyl-4-nitro-1H-pyrazol-1-yl)methyl]thiophene-2-carbonitrile (intermediate 91B) were reacted to give 188 mg (37%) of the desired title compound without purification.

¹H NMR (400 MHz, DMSO-d₆): δ (ppm)=1.99 (s, 3H), 2.06 (s, 3H), 3.43 (br. s., 2H), 5.33 (s, 2H), 7.10 (d, 1H), 7.80 (d, 1H).

### Intermediate 93C

### 5-{[4-amino-5-methyl-3-(trifluoromethyl)-1H-pyrazol-1-yl]methyl}thiophene-2-carbonitrile

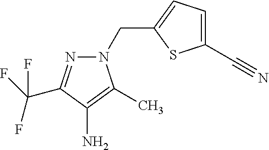 [SMILES: Cc1c(N)c(C(F)(F)F)nn1Cc1ccc(C#N)s1]

In analogy to intermediate 8C), 630 mg (1.79 mmol) 5-{[5-methyl-4-nitro-3-(trifluoromethyl)-1H-pyrazol-1-yl]methyl}thiophene-2-carbonitrile (intermediate 92B) were reacted to give 514 mg (65%) of the desired title compound without purification.

¹H NMR (400 MHz, DMSO-d₆): δ (ppm)=2.15 (s, 3H), 4.06 (br. s., 2H), 5.56 (s, 2H), 7.19 (d, 1H), 7.85 (d, 1H).

### Intermediate 94C

### 2-{[4-amino-5-methyl-3-(trifluoromethyl)-1H-pyrazol-1-yl]methyl}pyrimidine-5-carbonitrile

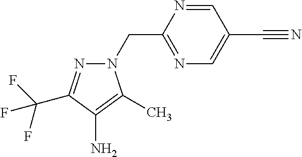 [SMILES: Cc1c(N)c(C(F)(F)F)nn1Cc1ncc(C#N)cn1]

In analogy to intermediate 10C), 2.40 g (7.69 mmol) of 2-{[5-methyl-4-nitro-3-(trifluoromethyl)-1H-pyrazol-1-yl]methyl}pyrimidine-5-carbonitrile (intermediate 93B) were reacted to give after purification of the crude product via a Biotage chromatography system (25 g snap KP-Sil column, hexane/0-100% ethyl acetate, then ethyl acetate/0-100% methanol) 490 mg (11%, purity 50%) of the desired title compound.

¹H-NMR (400 MHz, DMSO d₆) δ (ppm)=2.09 (s, 3H), 4.03 (s, 2H), 5.62 (s, 2H), 9.28 (s, 2H).

### Intermediate 95C

### 6-{[4-amino-5-methyl-3-(trifluoromethyl)-1H-pyrazol-1-yl]methyl}pyridazine-3-carbonitrile

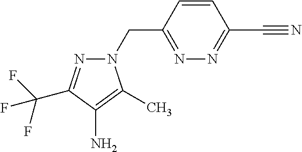 [SMILES: Cc1c(N)c(C(F)(F)F)nn1Cc1ccc(C#N)nn1]

In analogy to intermediate 10C), 400 mg (1.28 mmol) of 6-{[5-methyl-4-nitro-3-(trifluoromethyl)-1H-pyrazol-1-yl]methyl}pyridazine-3-carbonitrile (intermediate 94B) were reacted to give 400 mg (110%) crude product, which was used without any further purification

¹H-NMR (400 MHz, DMSO d₆) δ (ppm)=2.15 (s, 3H), 4.12 (s, 2H), 5.73 (s, 2H), 7.63 (d, 1H), 8.34 (d, 1H).

### Intermediate 1D

### 3,5-diethyl-4-nitro-1H-pyrazole

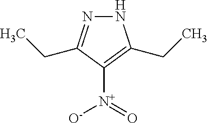

To a solution of 200 mg (1.61 mmol) commercially available 3,5-diethyl-1H-pyrazole in 0.71 mL conc. sulfuric acid was carefully added dropwise at 0° C. 0.26 mL 65% nitric acid. After stirring for 10 minutes the reaction mixture was heated up to 115° C. and stirring was continued for 4 hours at this temperature. After cooling to 25° C. the mixture was pured to 20 ml ice-water and extracted three times with ethyl acetate. The combined organic layer was washed with conc. aq. sodium bicarbonate, brine, dried over sodium sulfate, filtered and evaporated to obtain a crude product. An second experiment was done in the same manner with 1.23 g (9.90 mmol) 3,5-diethyl-1H-pyrazole. The combined crude products were purified via a Biotage chromatography system (25 g snap KP-Sil column, hexane/20-80% ethyl acetate) to obtain 1.61 g (83%) of the desired title compound.

¹H-NMR (400 MHz, DMSO d₆) δ (ppm)=1.20 (t, 6H), 2.89 (q, 4H), 13.37 (br. s., 1H).

### Intermediate 2D

### 5-ethyl-4-nitro-3-(trifluoromethyl)-1H-pyrazole

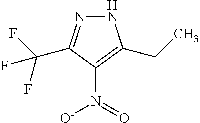

In analogy to intermediate 1D), 3.50 g (21.3 mmol) 5-ethyl-3-(trifluoromethyl)-1H-pyrazole (commercially available e.g. Aldrich, Princeton BioMolecular Research) were reacted to give after purification of the crude product via a Biotage chromatography system (50 g snap KP-Sil column, hexane/10-70% ethyl acetate) 4.27 g (96%) of the desired title compound.

¹H-NMR (400 MHz, DMSO d₆) δ (ppm)=1.25 (t, 3H), 3.00 (q, 2H).

### Intermediate 3D

### 5-isopropyl-4-nitro-3-(trifluoromethyl)-1H-pyrazole

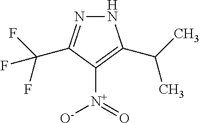

In analogy to intermediate 1D), 3.50 g (19.6 mmol) 5-isopropyl-3-(trifluoromethyl)-1H-pyrazole (commercially available e.g. Bellen Chemistry Co. or prepared according to US2011/105429, column 27) were reacted to give after purification of the crude product via a Biotage chromatography system (50 g snap KP-Sil column, hexane/10-100% ethyl acetate) 3.77 g (82%) of the desired title compound.

¹H-NMR (300 MHz, DMSO d₆) δ (ppm)=1.31 (d, 6H), 3.65 (spt, 1H), 14.43 (br. s., 1H).

### Intermediate 4D

### 3-isopropyl-5-methyl-4-nitro-1H-pyrazole

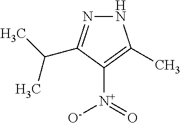

In analogy to intermediate 1D), in a first experiment 520 mg (4.19 mmol) and in two other experiments each with 1.62 g (13.0 mmol) 3-isopropyl-5-methyl-1H-pyrazole or its tautomer (commercially available e.g. Fluorochem) were reacted to give after purification of the crude product via a Biotage chromatography system (50 g snap KP-Sil column, hexane/20-70% ethyl acetate) 4.48 g (88%) of the desired title compound.

¹H-NMR (400 MHz, DMSO d₆) δ (ppm)=1.23 (d, 6H), 2.45 (br. s., 3H), 3.50 (br. s., 1H), 13.31 (br. s., 1H).

### Intermediate 5D

### 3-ethyl-5-methyl-4-nitro-1H-pyrazole

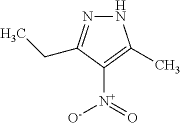

In analogy to intermediate 1D), 2.20 g (20.0 mmol) 3-ethyl-5-methyl-1H-pyrazole or its tautomer (preparation described in Chemische Berichte, 1928, p. 2406, 2410 or Journal fuer Praktische Chemie (Leipzig), 1930, p. 150) were reacted to give after purification of the crude product via a Biotage chromatography system (25 g snap KP-Sil column, hexane/10-70% ethyl acetate) 495 mg (15%) of the desired title compound.

¹H-NMR (300 MHz, DMSO d₆) δ (ppm)=1.18 (t, 3H), 2.44 (s, 3H), 2.86 (q, 2H), 13.34 (br. s., 1H).

### EXAMPLES

### Example 1

### 2-methoxy-N-[1-(4-methoxybenzyl)-3,5-dimethyl-1H-pyrazol-4-yl]quinoline-4-carboxamide

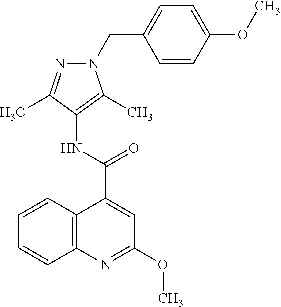 [SMILES: COc1ccc(Cn2nc(C)c(NC(=O)c3cc(OC)nc4ccccc34)c2C)cc1]

50 mg (0.22 mmol) 1-(4-methoxybenzyl)-3,5-dimethyl-1H-pyrazol-4-amine (intermediate 11C) was dissolved in 5 mL tetrahydrofuran and 52 mg (0.26 mmol) 2-methoxyquinoline-4-carboxylic acid, 56 μL (0.32 mmol) N,N-diisopropylethylamine and 104 mg (0.32 mmol) TBTU were added. The reaction mixture was stirred for 24 h at 25° C. After evaporation the residue was dissolved in 2.5 mL dimethylformamide and purified via preparative HPLC (method 3) to obtain 61 mg (0.14 mmol, 66%) of the desired title compound after drying.

1H NMR (300 MHz, DMSO d₆): δ (ppm)=2.12 (s, 3H), 2.16 (s, 3H), 3.73 (s, 3H), 4.04 (s, 3H), 5.16 (s, 2H), 6.91 (d, 2H), 7.14 (d, 2H), 7.21 (s, 1H), 7.47-7.55 (m, 1H), 7.69-7.77 (m, 1H), 7.83-7.89 (m, 1H), 8.01-8.08 (m, 1H), 9.88 (s, 1H).

### Example 2

### 6-bromo-N-[1-(4-fluorobenzyl)-3,5-dimethyl-1H-pyrazol-4-yl]-2-(propan-2-yl)quinoline-4-carboxamide

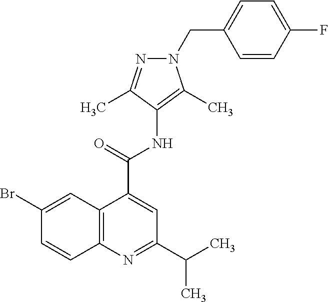 [SMILES: Cc1nn(Cc2ccc(F)cc2)c(C)c1NC(=O)c1cc(C(C)C)nc2ccc(Br)cc12]

In analogy to example 1), 75 mg (0.34 mmol) 1-(4-fluorobenzyl)-3,5-dimethyl-1H-pyrazol-4-amine (intermediate 1C) was stirred with 120 mg (0.41 mmol) 6-bromo-2-isopropylquinoline-4-carboxylic acid, 89 μL (0.51 mmol) N,N-diisopropylethylamine and 164 mg (0.51 mmol) TBTU in 5 mL tetrahydrofuran for 24 h at 25° C. The reaction mixture was evaporated and the residue partitioned between ethyl acetate and water. The layers were separated and the aqueous layer was extracted two further times with ethyl acetate. The combined organic layers were washed with brine, dried over sodium sulfate, filtered and evaporated. The residue was dissolved in dichloromethane and under evaporation adsorbed on Isolute® HM-N (Biotage). The isolute was given on a Biotage SNAP cartridge (25 g; KP-Sil) preequilibrated with hexane and purified via column chromatography on silica gel (solvent: hexane/0-100% ethyl acetate) to obtain 152 mg (0.31 mmol, 90%) of the desired title compound

1H NMR (300 MHz, DMSO d₆): δ (ppm)=1.35 (s, 3H), 1.38 (s, 3H), 2.14 (s, 3H), 2.18 (s, 3H), 3.23-3.32 (m, 1H), 5.25 (s, 2H), 7.15-7.27 (m, 4H), 7.75 (s, 1H), 7.88-7.94 (m, 1H), 7.98 (d, 1H), 8.33 (d, 1H), 9.97 (s, 1H).

### Example 3

### 6-bromo-N-[1-(4-methoxybenzyl)-3,5-dimethyl-1H-pyrazol-4-yl]-2-(trifluoromethyl)quinoline-4-carboxamide

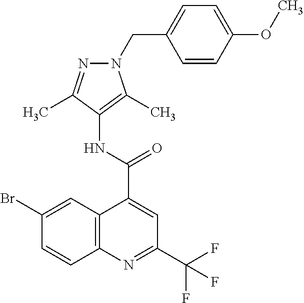 [SMILES: COc1ccc(Cn2nc(C)c(NC(=O)c3cc(C(F)(F)F)nc4ccc(Br)cc34)c2C)cc1]

In analogy to example 1), 50 mg (0.22 mmol) 1-(4-methoxybenzyl)-3,5-dimethyl-1H-pyrazol-4-amine (intermediate 11C) was stirred with 83 mg (0.26 mmol) 6-bromo-2-(trifluoromethyl)quinoline-4-carboxylic acid (intermediate 1A), 56 μL (0.32 mmol) N,N-diisopropylethylamine and 104 mg (0.32 mmol) TBTU in 5 mL tetrahydrofuran for 24 h at 25° C. to obtain 78 mg (0.14 mmol, 67%) of the desired title compound after preparative HPLC (method 3).

1H NMR (300 MHz, DMSO d₆): δ (ppm)=2.14 (s, 3H), 2.19 (s, 3H), 3.71-3.77 (m, 3H), 5.18 (s, 2H), 6.92 (d, 2H), 7.15 (d, 2H), 8.11-8.19 (m, 1H), 8.23 (d, 1H), 8.28 (s, 1H), 8.50 (d, 1H), 10.13 (s, 1H).

### Example 4

### N-[1-(4-methoxybenzyl)-3,5-dimethyl-1H-pyrazol-4-yl]-2,6-dimethylquinoline-4-carboxamide

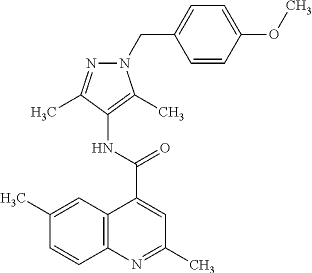

In analogy to example 1), 50 mg (0.22 mmol) 1-(4-methoxybenzyl)-3,5-dimethyl-1H-pyrazol-4-amine (intermediate 11C) was stirred with 52 mg (0.26 mmol) 2,6-dimethylquinoline-4-carboxylic acid, 56 μL (0.32 mmol) N,N-diisopropylethylamine and 104 mg (0.32 mmol) TBTU in 5 mL tetrahydrofuran for 24 h at 25° C. to obtain 47 mg (0.11 mmol, 52%) of the desired title compound after preparative HPLC (method 3).

1H NMR (300 MHz, DMSO d₆): δ (ppm)=2.13 (s, 3H), 2.17 (s, 3H), 2.69 (s, 3H), 3.73 (s, 3H), 5.16 (s, 2H), 6.91 (d, 2H), 7.16 (d, 2H), 7.56 (s, 1H), 7.61 (dd, 1H), 7.85-7.92 (m, 2H), 9.84 (s, 1H).

### Example 5

### 6-bromo-N-[1-(4-cyanobenzyl)-3,5-dimethyl-1H-pyrazol-4-yl]-2-(trifluoromethyl)quinoline-4-carboxamide

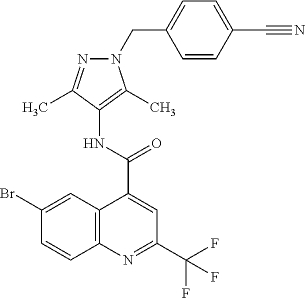 [SMILES: Cc1nn(Cc2ccc(C#N)cc2)c(C)c1NC(=O)c1cc(C(F)(F)F)nc2ccc(Br)cc12]

In analogy to example 1), 50 mg (0.22 mmol) 4-[(4-amino-3,5-dimethyl-1H-pyrazol-1-yl)methyl]benzonitrile (intermediate 8C) was stirred with 84 mg (0.27 mmol) 6-bromo-2-(trifluoromethyl)quinoline-4-carboxylic acid (intermediate 1A), 58 μL (0.33 mmol) N,N-diisopropylethylamine and 106 mg (0.33 mmol) TBTU in 5 mL tetrahydrofuran for 24 h at 25° C. to obtain 26 mg (0.04 mmol, 21%) of the desired title compound after preparative HPLC (method 3).

1H NMR (300 MHz, DMSO d₆): δ (ppm)=2.16 (s, 3H), 2.19 (s, 3H), 5.39 (s, 2H), 7.32 (d, 2H), 7.85 (d, 2H), 8.11-8.19 (m, 1H), 8.23 (d, 1H), 8.30 (s, 1H), 8.51 (d, 1H), 10.19 (s, 1H).

### Example 6

### 6-bromo-N-[1-(2-cyanobenzyl)-3,5-dimethyl-1H-pyrazol-4-yl]-2-(trifluoromethyl)quinoline-4-carboxamide

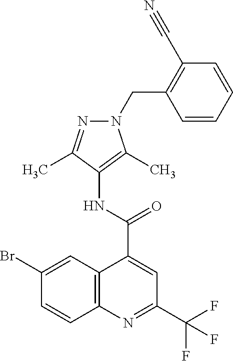 [SMILES: Cc1nn(Cc2ccccc2C#N)c(C)c1NC(=O)c1cc(C(F)(F)F)nc2ccc(Br)cc12]

In analogy to example 1), 50 mg (0.22 mmol) 2-[(4-amino-3,5-dimethyl-1H-pyrazol-1-yl)methyl]benzonitrile (intermediate 10C) was stirred with 84 mg (0.27 mmol) 6-bromo-2-(trifluoromethyl)quinoline-4-carboxylic acid (intermediate 1A), 58 μL (0.33 mmol) N,N-diisopropylethylamine and 106 mg (0.33 mmol) TBTU in 5 mL tetrahydrofuran for 24 h at 25° C. to obtain 67 mg (0.13 mmol, 57%) of the desired title compound after preparative HPLC (method 3).

1H NMR (400 MHz, DMSO d₆): δ (ppm)=2.14 (s, 3H), 2.27 (s, 3H), 5.45 (s, 2H), 7.11 (d, 1H), 7.50-7.56 (m, 1H), 7.72 (td, 1H), 7.90 (dd, 1H), 8.15 (dd, 1H), 8.23 (d, 1H), 8.31 (s, 1H), 8.52 (d, 1H), 10.22 (s, 1H).

### Example 7

### 2-cyclopropyl-N-[1-(4-fluorobenzyl)-3,5-dimethyl-1H-pyrazol-4-yl]-6-methylquinoline-4-carboxamide

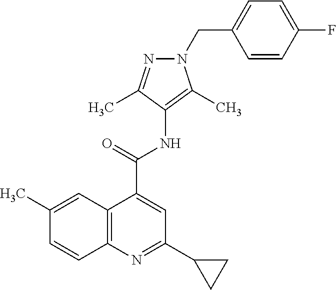 [SMILES: Cc1ccc2nc(C3CC3)cc(C(=O)Nc3c(C)nn(Cc4ccc(F)cc4)c3C)c2c1]

In analogy to example 1), 100 mg (0.45 mmol) 1-(4-fluorobenzyl)-3,5-dimethyl-1H-pyrazol-4-amine (intermediate 1C) was stirred with 124 mg (0.54 mmol) 2-cyclopropyl-6-methylquinoline-4-carboxylic acid, 119 μL (0.68 mmol) N,N-diisopropylethylamine and 220 mg (0.68 mmol) TBTU in 5 mL tetrahydrofuran for 24 h at 25° C. The reaction mixture was evaporated and the residue partitioned between ethyl acetate and water. The layers were separated and the aqueous layer was extracted two further times with ethyl acetate. The combined organic layers were washed with brine, dried over sodium sulfate, filtered and evaporated. The residue was dissolved in dichloromethane and under evaporation adsorbed on Isolute® HM-N (Biotage). The isolute was given on a Biotage SNAP cartridge (25 g; KP-Sil) preequilibrated with hexane and purified via column chromatography on silica gel (solvent: hexane/0-100% ethyl acetate). The resulting crude product was again purified via preparative HPLC (method 3) to obtain 100 mg (0.43 mmol, 51%) of the desired title compound.

1H NMR (400 MHz, DMSO d₆): δ (ppm)=1.04-1.10 (m, 2H), 1.12 (dt, 2H), 2.14 (s, 3H), 2.18 (s, 3H), 2.31-2.39 (m, 1H), 2.47 (s, 3H), 5.24 (s, 2H), 7.16-7.28 (m, 4H), 7.54-7.59 (m, 2H), 7.81 (d, 1H), 7.85 (s, 1H), 9.83 (s, 1H).

### Example 8

### 6,8-dichloro-N-[1-(4-methoxybenzyl)-3,5-dimethyl-1H-pyrazol-4-yl]-2-(trifluoromethyl)quinoline-4-carboxamide

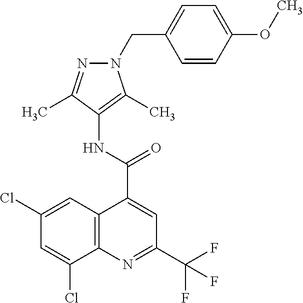 [SMILES: COc1ccc(Cn2nc(C)c(NC(=O)c3cc(C(F)(F)F)nc4c(Cl)cc(Cl)cc34)c2C)cc1]

In analogy to example 1), 50 mg (0.22 mmol) 1-(4-methoxybenzyl)-3,5-dimethyl-1H-pyrazol-4-amine (intermediate 11C) was stirred with 80 mg (0.26 mmol) 6,8-dichloro-2-(trifluoromethyl)quinoline-4-carboxylic acid (intermediate 11A), 56 μL (0.33 mmol) N,N-diisopropylethylamine and 104 mg (0.32 mmol) TBTU in 5 mL tetrahydrofuran for 24 h at 25° C. to obtain 59 mg (0.11 mmol, 52%) of the desired title compound after preparative HPLC (method 3).

1H NMR (300 MHz, DMSO d₆): δ (ppm)=2.14 (s, 3H), 2.18 (s, 3H), 3.73 (s, 3H), 5.17 (s, 2H), 6.91 (m, 2H), 7.15 (m, 2H), 8.29 (d, 1H), 8.39-8.43 (m, 2H), 10.19 (s, 1H).

### Example 9

### 6-bromo-N-[1-(4-fluorobenzyl)-3,5-dimethyl-1H-pyrazol-4-yl]-2-(trifluoromethyl)quinoline-4-carboxamide

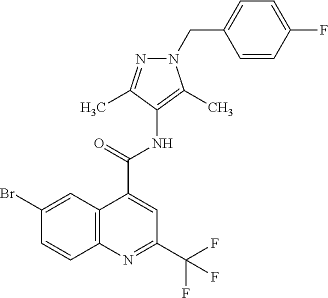 [SMILES: Cc1nn(Cc2ccc(F)cc2)c(C)c1NC(=O)c1cc(C(F)(F)F)nc2ccc(Br)cc12]

In analogy to example 1), 150 mg (0.68 mmol) 1-(4-fluorobenzyl)-3,5-dimethyl-1H-pyrazol-4-amine (intermediate 1C) was stirred with 262 mg (0.82 mmol) 6-bromo-2-(trifluoromethyl)quinoline-4-carboxylic acid (intermediate 1A), 179 μL (1.03 mmol) N,N-diisopropylethylamine and 329 mg (1.03 mmol) TBTU in 15 mL tetrahydrofuran for 24 h at 25° C. The reaction mixture was evaporated and the residue was dissolved in dichloromethane and under evaporation adsorbed on Isolute® HM-N (Biotage). The isolute was given on a Biotage SNAP cartridge (25 g; KP-Sil) preequilibrated with hexane and purified via column chromatography on silica gel (solvent: hexane/0-80% ethyl acetate) to obtain 286 mg (0.55 mmol, 80%) of the desired title compound.

1H NMR (300 MHz, DMSO d₆): δ (ppm)=2.15 (s, 3H), 2.19 (s, 3H), 5.25 (s, 2H), 7.13-7.29 (m, 4H), 8.15 (dd, 1H), 8.23 (d, 1H), 8.29 (s, 1H), 8.50 (d, 1H), 10.17 (s, 1H).

### Example 10

### 2-cyclopropyl-6-fluoro-N-[1-(4-fluorobenzyl)-3,5-dimethyl-1H-pyrazol-4-yl]quinoline-4-carboxamide

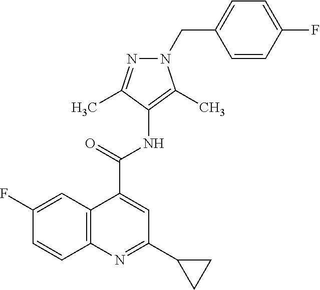 [SMILES: Cc1nn(Cc2ccc(F)cc2)c(C)c1NC(=O)c1cc(C2CC2)nc2ccc(F)cc12]

In analogy to example 1), 75 mg (0.34 mmol) 1-(4-fluorobenzyl)-3,5-dimethyl-1H-pyrazol-4-amine (intermediate 1C) was stirred with 94 mg (0.41 mmol) 2-cyclopropyl-6-fluoroquinoline-4-carboxylic acid (intermediate 26A), 89 μL (0.51 mmol) N,N-diisopropylethylamine and 164 mg (0.51 mmol) TBTU in 5 mL tetrahydrofuran for 24 h at 25° C. to obtain 72 mg (0.16 mmol, 48%) of the desired title compound after preparative HPLC (method 3).

1H NMR (300 MHz, DMSO d₆): δ (ppm)=1.07-1.19 (m, 4H), 2.13 (s, 3H), 2.17 (s, 3H), 2.33-2.44 (m, 1H), 5.24 (s, 2H), 7.10-7.32 (m, 4H), 7.61-7.70 (m, 1H), 7.72 (s, 1H), 7.81 (dd, 1H), 7.99 (dd, 1H), 9.92 (s, 1H).

### Example 11

### 6,8-dichloro-N-[1-(4-cyanobenzyl)-3,5-dimethyl-1H-pyrazol-4-yl]-2-(trifluoromethyl)quinoline-4-carboxamide

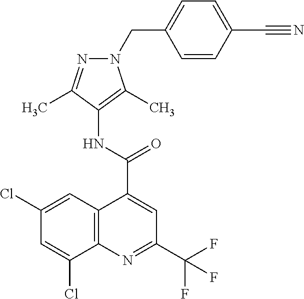 [SMILES: Cc1nn(Cc2ccc(C#N)cc2)c(C)c1NC(=O)c1cc(C(F)(F)F)nc2c(Cl)cc(Cl)cc12]

In analogy to example 1), 50 mg (0.22 mmol) 4-[(4-amino-3,5-dimethyl-1H-pyrazol-1-yl)methyl]benzonitrile (intermediate 8C) was stirred with 82 mg (0.27 mmol) 6,8-dichloro-2-(trifluoromethyl)quinoline-4-carboxylic acid (intermediate 11A), 58 μL (0.33 mmol) N,N-diisopropylethylamine and 106 mg (0.33 mmol) TBTU in 5 mL tetrahydrofuran for 24 h at 25° C. to obtain 32 mg (0.06 mmol, 28%) of the desired title compound after preparative HPLC (method 3).

1H NMR (300 MHz, DMSO d₆): δ (ppm)=2.16 (s, 3H), 2.18 (s, 3H), 5.38 (s, 2H), 7.32 (d, 2H), 7.85 (d, 2H), 8.30 (d, 1H), 8.40 (d, 1H), 8.42 (s, 1H), 10.23 (s, 1H).

### Example 12

### 6-bromo-2-cyclopropyl-N-[1-(4-fluorobenzyl)-3,5-dimethyl-1H-pyrazol-4-yl]quinoline-4-carboxamide

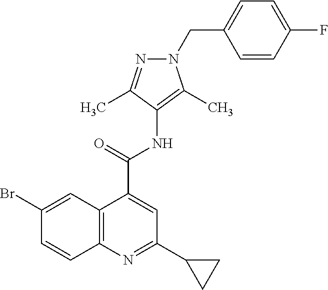 [SMILES: Cc1nn(Cc2ccc(F)cc2)c(C)c1NC(=O)c1cc(C2CC2)nc2ccc(Br)cc12]

In analogy to example 1), 100 mg (0.45 mmol) 1-(4-fluorobenzyl)-3,5-dimethyl-1H-pyrazol-4-amine (intermediate 1C) was stirred with 159 mg (0.54 mmol) 6-bromo-2-cyclopropylquinoline-4-carboxylic acid, 119 μL (0.68 mmol) N,N-diisopropylethylamine and 220 mg (0.68 mmol) TBTU in 5 mL tetrahydrofuran for 24 h at 25° C. The reaction mixture was evaporated and the residue partitioned between ethyl acetate and water. The layers were separated and the aqueous layer was extracted two further times with ethyl acetate. The combined organic layers were washed with brine, dried over sodium sulfate, filtered and evaporated. The resulting crude product was purified via preparative HPLC (method 3) to obtain 135 mg (0.28 mmol, 60%) of the desired title compound.

1H NMR (400 MHz, DMSO d₆): δ (ppm)=1.09-1.19 (m, 4H), 2.13 (s, 3H), 2.17 (s, 3H), 2.35-2.44 (m, 1H), 5.24 (s, 2H), 7.15-7.28 (m, 4H), 7.72 (s, 1H), 7.87 (d, 2H), 8.29 (t, 1H), 9.95 (s, 1H).

### Example 13

### 6-bromo-N-[1-(2, 6-difluorobenzyl)-3,5-dimethyl-1H-pyrazol-4-yl]-2-(trifluoromethyl)quinoline-4-carboxamide

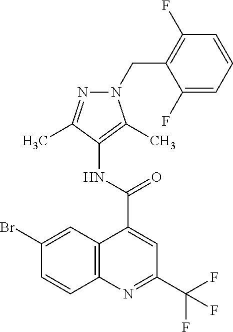 [SMILES: Cc1nn(Cc2c(F)cccc2F)c(C)c1NC(=O)c1cc(C(F)(F)F)nc2ccc(Br)cc12]

In analogy to example 1), 50 mg (0.21 mmol) 1-(2,6-difluorobenzyl)-3,5-dimethyl-1H-pyrazol-4-amine (intermediate 6C) was stirred with 80 mg (0.25 mmol) 6-bromo-2-(trifluoromethyl)quinoline-4-carboxylic acid (intermediate 1A), 55 μL (0.32 mmol) N,N-diisopropylethylamine and 102 mg (0.32 mmol) TBTU in 3 mL tetrahydrofuran for 24 h at 25° C. The reaction mixture was diluted with dichloromethane, filtered, washed with dichloromethane and dried in high vacuum to obtain 57 mg (0.11 mmol, 50%) of the desired title compound.

1H NMR (300 MHz, DMSO d₆): δ (ppm)=2.07 (s, 3H), 2.32 (s, 3H), 5.25 (s, 2H), 7.15 (t, 2H), 7.41-7.54 (m, 1H), 8.15 (dd, 1H), 8.23 (d, 1H), 8.30 (s, 1H), 8.49 (d, 1H), 10.15 (s, 1H).

### Example 14

### 6-chloro-N-[1-(4-fluorobenzyl)-3,5-dimethyl-1H-pyrazol-4-yl]-2-(trifluoromethyl)quinoline-4-carboxamide

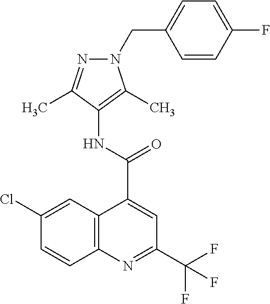 [SMILES: Cc1nn(Cc2ccc(F)cc2)c(C)c1NC(=O)c1cc(C(F)(F)F)nc2ccc(Cl)cc12]

In analogy to example 1), 60 mg (0.27 mmol) 1-(4-fluorobenzyl)-3,5-dimethyl-1H-pyrazol-4-amine (intermediate 1C) was stirred with 91 mg (0.33 mmol) 6-chloro-2-(trifluoromethyl)quinoline-4-carboxylic acid (intermediate 12A), 71 μL (0.41 mmol) N,N-diisopropylethylamine and 132 mg (0.41 mmol) TBTU in 5 mL tetrahydrofuran for 24 h at 25° C. to obtain 112 mg (0.23 mmol, 86%) of the desired title compound after preparative HPLC (method 3).

1H NMR (300 MHz, DMSO d₆): δ (ppm)=2.15 (s, 3H), 2.19 (s, 3H), 5.25 (s, 2H), 7.14-7.29 (m, 4H), 8.04 (dd, 1H) 8.27-8.37 (m, 3H), 10.16 (s, 1H).

### Example 15

### N-[1-(4-cyanobenzyl)-3,5-dimethyl-1H-pyrazol-4-yl]-2-methoxyquinoline-4-carboxamide

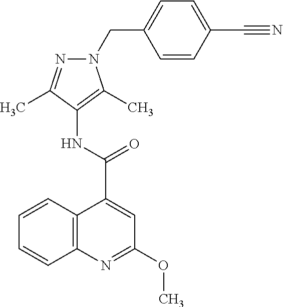 [SMILES: COc1cc(C(=O)Nc2c(C)nn(Cc3ccc(C#N)cc3)c2C)c2ccccc2n1]

In analogy to example 1), 50 mg (0.22 mmol) 4-[(4-amino-3,5-dimethyl-1H-pyrazol-1-yl)methyl]benzonitrile (intermediate 8C) was stirred with 54 mg (0.27 mmol) 2-methoxyquinoline-4-carboxylic acid, 58 μL (0.33 mmol) N,N-diisopropylethylamine and 106 mg (0.33 mmol) TBTU in 5 mL tetrahydrofuran for 24 h at 25° C. to obtain 29 mg (0.07 mmol, 31%) of the desired title compound after preparative HPLC (method 3).

1H NMR (300 MHz, DMSO d₆): δ (ppm)=2.14 (s, 3H), 2.16 (s, 3H), 4.04 (s, 3H), 5.37 (s, 2H), 7.23 (s, 1H), 7.31 (d, 2H), 7.51 (td, 1H), 7.73 (td, 1H), 7.80-7.90 (m, 3H), 8.06 (d, 1H), 9.91 (s, 1H).

### Example 16

### 8-bromo-2-cyclopropyl-N-[1-(4-fluorobenzyl)-3,5-dimethyl-1H-pyrazol-4-yl]quinoline-4-carboxamide

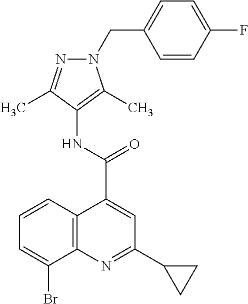 [SMILES: Cc1nn(Cc2ccc(F)cc2)c(C)c1NC(=O)c1cc(C2CC2)nc2c(Br)cccc12]

In analogy to example 1), 60 mg (0.27 mmol) 1-(4-fluorobenzyl)-3,5-dimethyl-1H-pyrazol-4-amine (intermediate 1C) was stirred with 96 mg (0.33 mmol) 8-bromo-2-cyclopropylquinoline-4-carboxylic acid (intermediate 27A), 71 μL (0.41 mmol) N,N-diisopropylethylamine and 132 mg (0.41 mmol) TBTU in 5 mL tetrahydrofuran for 24 h at 25° C. to obtain 75 mg (0.15 mmol, 56%) of the desired title compound after preparative HPLC (method 3).

1H NMR (400 MHz, DMSO d₆): δ (ppm)=1.13-1.23 (m, 4H), 2.14 (s, 3H), 2.18 (s, 3H), 2.41-2.47 (m, 1H), 5.24 (s, 2H), 7.15-7.26 (m, 4H), 7.47 (dd, 1H), 7.74 (s, 1H), 8.08 (dd, 1H), 8.13 (dd, 1H), 9.92 (s, 1H).

### Example 17

### N-[1-(2,4-difluorobenzyl)-3,5-dimethyl-1H-pyrazol-4-yl]-2-methoxyquinoline-4-carboxamide

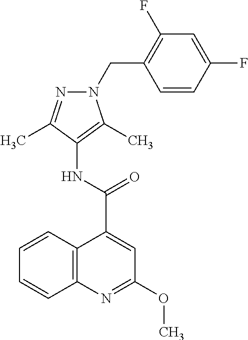 [SMILES: COc1cc(C(=O)Nc2c(C)nn(Cc3ccc(F)cc3F)c2C)c2ccccc2n1]

In analogy to example 1), 50 mg (0.21 mmol) 1-(2,4-difluorobenzyl)-3,5-dimethyl-1H-pyrazol-4-amine (intermediate 5C) was stirred with 51 mg (0.25 mmol) 2-methoxyquinoline-4-carboxylic acid, 55 μL (0.31 mmol) N,N-diisopropylethylamine and 101 mg (0.32 mmol) TBTU in 5 mL tetrahydrofuran for 24 h at 25° C. to obtain 10 mg (0.02 mmol, 11%) of the desired title compound after preparative HPLC (method 3).

1H NMR (300 MHz, DMSO d₆): δ (ppm)=2.11 (s, 3H), 2.21 (s, 3H), 4.04 (s, 3H), 5.25 (s, 2H), 7.05-7.18 (m, 2H), 7.22 (s, 1H), 7.23-7.33 (m, 1H), 7.47-7.55 (m, 1H), 7.73 (td, 1H), 7.83-7.89 (m, 1H), 8.05 (d, 1H), 9.89 (s, 1H).

### Example 18

### 8-bromo-N-[1-(4-fluorobenzyl)-3,5-dimethyl-1H-pyrazol-4-yl]-2-(trifluoromethyl)quinoline-4-carboxamide

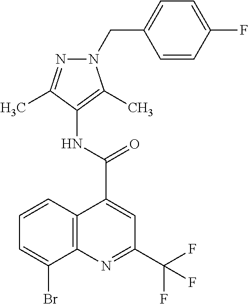 [SMILES: Cc1nn(Cc2ccc(F)cc2)c(C)c1NC(=O)c1cc(C(F)(F)F)nc2c(Br)cccc12]

In analogy to example 1), 150 mg (0.68 mmol) 1-(4-fluorobenzyl)-3,5-dimethyl-1H-pyrazol-4-amine (intermediate 1C) was stirred with 263 mg (0.82 mmol) 8-bromo-2-(trifluoromethyl)quinoline-4-carboxylic acid (intermediate 13A), 179 μL (1.03 mmol) N,N-diisopropylethylamine and 329 mg (1.03 mmol) TBTU in 5 mL tetrahydrofuran for 24 h at 25° C. The reaction mixture was evaporated, dissolved in dichloromethane and under evaporation adsorbed on Isolute® HM-N (Biotage). The isolute was given on a Biotage SNAP cartridge (25 g; KP-Sil) preequilibrated with hexane and purified via column chromatography on silica gel (solvent: hexane/0-80% ethyl acetate). The resulting crude product was again purified via preparative HPLC (method 3) to obtain 280 mg (0.54 mmol, 79%) of the desired title compound.

1H NMR (300 MHz, DMSO d₆): δ (ppm)=2.16 (s, 3H), 2.20 (s, 3H), 5.25 (s, 2H), 7.15-7.27 (m, 4H), 7.76-7.84 (m, 1H), 8.27 (dd, 1H), 8.32 (s, 1H), 8.41 (dd, 1H), 10.14 (s, 1H).

### Example 19

### 2-cyclopropyl-N-[1-(4-fluorobenzyl)-3,5-dimethyl-1H-pyrazol-4-yl]quinoline-4-carboxamide

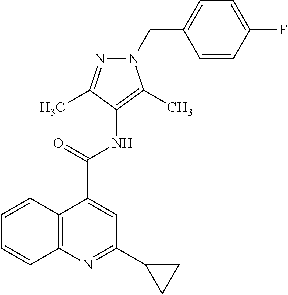 [SMILES: Cc1nn(Cc2ccc(F)cc2)c(C)c1NC(=O)c1cc(C2CC2)nc2ccccc12]

In analogy to example 1), 100 mg (0.46 mmol) 1-(4-fluorobenzyl)-3,5-dimethyl-1H-pyrazol-4-amine (intermediate 1C) was stirred with 117 mg (0.55 mmol) 2-cyclopropylquinoline-4-carboxylic acid, 119 μL (0.68 mmol) N,N-diisopropylethylamine and 220 mg (0.68 mmol) TBTU in 5 mL tetrahydrofuran for 3 h at 25° C. The reaction mixture was evaporated and the residue partitioned between ethyl acetate and water. The layers were separated and the aqueous layer was extracted two further times with ethyl acetate. The combined organic layers were washed with brine, dried over sodium sulfate, filtered and evaporated. The residue was dissolved in dichloromethane and under evaporation adsorbed on Isolute® HM-N (Biotage). The isolute was given on a Biotage SNAP cartridge (25 g; KP-Sil) preequilibrated with hexane and purified via column chromatography on silica gel (solvent: hexane/0 100% ethyl acetate). The resulting crude product was again purified via preparative HPLC (method 3) to obtain 100 mg (0.43 mmol, 51%) of the desired title compound.

1H NMR (400 MHz, DMSO ds): δ (ppm)=1.07-1.13 (m, 2H), 1.13-1.18 (m, 2H), 2.14 (s, 3H), 2.18 (s, 3H), 2.34-2.43 (m, 1H), 5.24 (s, 2H), 7.15-7.27 (m, 4H), 7.56 (ddd, 1H), 7.62 (s, 1H), 7.73 (ddd, 1H), 7.91 (d, 1H), 8.06-8.11 (m, 1H), 9.86 (s, 1H).

### Example 20

### 6,8-dichloro-N-[1-(2, 6-difluorobenzyl)-3,5-dimethyl-1H-pyrazol-4-yl]-2-(trifluoromethyl)quinoline-4-carboxamide

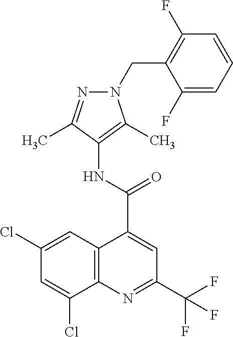 [SMILES: Cc1nn(Cc2c(F)cccc2F)c(C)c1NC(=O)c1cc(C(F)(F)F)nc2c(Cl)cc(Cl)cc12]

In analogy to example 1), 50 mg (0.21 mmol) 1-(2,6-difluorobenzyl)-3,5-dimethyl-1H-pyrazol-4-amine (intermediate 6C) was stirred with 78 mg (0.25 mmol) 6,8-dichloro-2-(trifluoromethyl)quinoline-4-carboxylic acid (intermediate 1A), 55 μL (0.31 mmol) N,N-diisopropylethylamine and 101 mg (0.31 mmol) TBTU in 5 mL tetrahydrofuran for 24 h at 25° C. to obtain 75 mg (0.13 mmol, 64%) of the desired title compound after preparative HPLC (method 3).

1H NMR (300 MHz, DMSO d₆): δ (ppm)=2.06 (s, 3H), 2.32 (s, 3H), 5.24 (s, 2H), 7.15 (t, 2H), 7.41-7.54 (m, 1H), 8.28 (d, 1H), 8.38-8.45 (m, 2H), 10.20 (s, 1H).

### Example 21

### 8-bromo-N-[1-(4-fluorobenzyl)-3,5-dimethyl-1H-pyrazol-4-yl]-6-methyl-2-(trifluoromethyl)quinoline-4-carboxamide

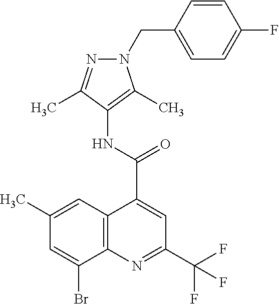 [SMILES: Cc1cc(Br)c2nc(C(F)(F)F)cc(C(=O)Nc3c(C)nn(Cc4ccc(F)cc4)c3C)c2c1]

In analogy to example 1), 150 mg (0.68 mmol) 1-(4-fluorobenzyl)-3,5-dimethyl-1H-pyrazol-4-amine (intermediate 1C) was stirred with 274 mg (0.82 mmol) 8-bromo-6-methyl-2-(trifluoromethyl)quinoline-4-carboxylic acid (intermediate 14A), 179 μL (1.03 mmol) N,N-diisopropylethylamine and 329 mg (1.03 mmol) TBTU in 5 mL tetrahydrofuran for 24 h at 25° C. The reaction mixture was evaporated, dissolved in dichloromethane and under evaporation adsorbed on Isolute® HM-N (Biotage). The isolute was given on a Biotage SNAP cartridge (25 g; KP-Sil) preequilibrated with hexane and purified via column chromatography on silica gel (solvent: hexane/0-80% ethyl acetate) to obtain 289 mg (0.54 mmol, 79%) of the desired title compound.

1H NMR (400 MHz, DMSO d₆): δ (ppm)=1.35 (s, 3H), 1.39 (s, 3H), 1.75 (s, 3H), 4.44 (s, 2H), 6.31-6.50 (m, 4H), 7.17-7.26 (m, 1H), 7.44 (s, 1H), 7.48 (d, 1H), 9.28 (s, 1H).

### Example 22

### 5,6-dichloro-N-[1-(4-fluorobenzyl)-3,5-dimethyl-1H-pyrazol-4-yl]-2-(trifluoromethyl)quinoline-4-carboxamide

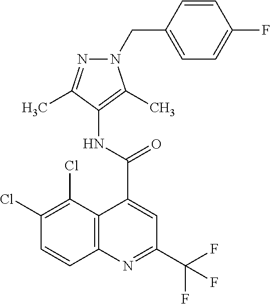 [SMILES: Cc1nn(Cc2ccc(F)cc2)c(C)c1NC(=O)c1cc(C(F)(F)F)nc2ccc(Cl)c(Cl)c12]

In analogy to example 1), 50 mg (0.23 mmol) 1-(4-fluorobenzyl)-3,5-dimethyl-1H-pyrazol-4-amine (intermediate 1C) was stirred with 84 mg (0.27 mmol) of a mixture of 5,6-dichloro-2-(trifluoromethyl)quinoline-4-carboxylic acid and 6,7-dichloro-2-(trifluoromethyl)quinoline-4-carboxylic acid (3:1) (intermediate 15A), 60 μL (0.34 mmol) N,N-diisopropylethylamine and 109 mg (0.34 mmol) TBTU in 5 mL tetrahydrofuran for 24 h at 25° C. to obtain 32 mg (0.06 mmol, 27%) of the desired title compound after preparative HPLC (method 6). Furthermore 23 mg (0.04 mmol, 20%) of 6,7-dichloro-N-[1-(4-fluorobenzyl)-3,5-dimethyl-1H-pyrazol-4-yl]-2-(trifluoromethyl)quinoline-4-carboxamide (example 106) were isolated after preparative HPLC.

1H NMR (400 MHz, DMSO d₆): δ (ppm)=2.17 (s, 3H), 2.22 (s, 3H), 5.23 (s, 2H), 7.14-7.27 (m, 4H), 8.20 (s, 1H), 8.24 (d, 1H), 8.31 (d, 1H), 10.01 (s, 1H).

### Example 23

### N-{3,5-dimethyl-1-[4-(trifluoromethoxy)benzyl]-1H-pyrazol-4-yl}-2-methoxyquinoline-4-carboxamide

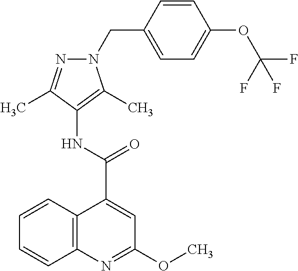 [SMILES: COc1cc(C(=O)Nc2c(C)nn(Cc3ccc(OC(F)(F)F)cc3)c2C)c2ccccc2n1]

In analogy to example 1), 100 mg (0.18 mmol, 50%) 3,5-dimethyl-1-[4-(trifluoromethoxy)benzyl]-1H-pyrazol-4-amine (intermediate 19C) was stirred with 43 mg (0.21 mmol) 2-methoxyquinoline-4-carboxylic acid, 46 μL (0.26 mmol) N,N-diisopropylethylamine and 84 mg (0.26 mmol) TBTU in 3 mL tetrahydrofuran for 24 h at 25° C. to obtain 68 mg (0.14 mmol, 81%) of the desired title compound after preparative HPLC (method 3).

1H NMR (300 MHz, DMSO d₆): δ (ppm)=2.13 (s, 3H), 2.18 (s, 3H), 4.04 (s, 3H), 5.29 (s, 2H), 7.22 (s, 1H), 7.30 (d, 2H), 7.37 (d, 2H), 7.48-7.55 (m, 1H), 7.70-7.77 (m, 1H), 7.86 (d, 1H), 8.05 (d, 1H), 9.91 (s, 1H).

### Example 24

### 6-bromo-N-[3,5-dimethyl-1-(2-methylbenzyl)-1H-pyrazol-4-yl]-2-(trifluoromethyl)quinoline-4-carboxamide

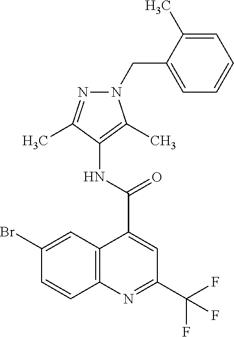 [SMILES: Cc1ccccc1Cn1nc(C)c(NC(=O)c2cc(C(F)(F)F)nc3ccc(Br)cc23)c1C]

In analogy to example 1), 50 mg (0.23 mmol, 50%) 3,5-dimethyl-1-(2-methylbenzyl)-1H-pyrazol-4-amine (intermediate 15C) was stirred with 89 mg (0.28 mmol) 6-bromo-2-(trifluoromethyl)quinoline-4-carboxylic acid (intermediate 1A), 49 μL (0.35 mmol) N,N-diisopropylethylamine and 112 mg (0.35 mmol) TBTU in 3 mL tetrahydrofuran for 24 h at 25° C. to obtain 48 mg (0.09 mmol, 38%) of the desired title compound after preparative HPLC (method 3).

1H NMR (300 MHz, DMF): δ (ppm)=2.27 (d, 6H), 2.42 (s, 3H), 5.36 (s, 2H), 6.74 (d, 1H), 7.13-7.28 (m, 3H), 8.20 (dd, 1H), 8.27 (d, 1H), 8.43 (s, 1H), 8.71 (d, 1H), 10.29 (s, 1H).

### Example 25

### 6,8-dichloro-N-[1-(4-fluorobenzyl)-3,5-dimethyl-1H-pyrazol-4-yl]-2-(trifluoromethyl)quinoline-4-carboxamide

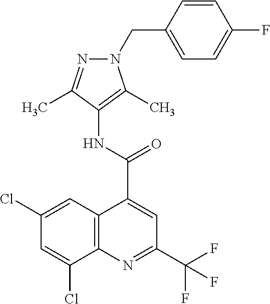 [SMILES: Cc1nn(Cc2ccc(F)cc2)c(C)c1NC(=O)c1cc(C(F)(F)F)nc2c(Cl)cc(Cl)cc12]

In analogy to example 1), 50 mg (0.23 mmol) 1-(4-fluorobenzyl)-3,5-dimethyl-1H-pyrazol-4-amine (intermediate 1C) was stirred with 85 mg (0.27 mmol) 6,8-dichloro-2-(trifluoromethyl)quinoline-4-carboxylic acid (intermediate 11A), 60 μL (0.34 mmol) N,N-diisopropylethylamine and 110 mg (0.34 mmol) TBTU in 3 mL tetrahydrofuran for 24 h at 25° C. to obtain 75 mg (0.15 mmol, 64%) of the desired title compound after preparative HPLC (method 3).

1H NMR (300 MHz, DMSO d₆): δ (ppm)=2.15 (s, 3H), 2.19 (s, 3H), 5.25 (s, 2H), 7.14-7.29 (m, 4H), 8.29 (d, 1H), 8.41 (d, 1H), 8.42 (s, 1H), 10.21 (s, 1H).

### Example 26

### 5-fluoro-N-[1-(4-fluorobenzyl)-3,5-dimethyl-1H-pyrazol-4-yl]-2-(trifluoromethyl)quinoline-4-carboxamide

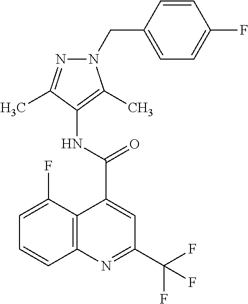 [SMILES: Cc1nn(Cc2ccc(F)cc2)c(C)c1NC(=O)c1cc(C(F)(F)F)nc2cccc(F)c12]

In analogy to example 1), 50 mg (0.23 mmol) 1-(4-fluorobenzyl)-3,5-dimethyl-1H-pyrazol-4-amine (intermediate 1C) was stirred with 71 mg (0.27 mmol) 5-fluoro-2-(trifluoromethyl)quinoline-4-carboxylic acid (intermediate 16A), 60 μL (0.34 mmol) N,N-diisopropylethylamine and 110 mg (0.34 mmol) TBTU in 3 mL tetrahydrofuran for 24 h at 25° C. to obtain 98 mg (0.21 mmol, 92%) of the desired title compound after preparative HPLC (method 3).

1H NMR (300 MHz, DMSO d₆): δ (ppm)=2.15 (s, 3H), 2.19 (s, 3H), 5.25 (s, 2H), 7.13-7.27 (m, 4H), 7.87 (td, 1H), 8.09 (dd, 1H), 8.20 (s, 1H), 8.38 (dd, 1H), 10.12 (s, 1H).

### Example 27

### N-[1-(4-fluorobenzyl)-3,5-dimethyl-1H-pyrazol-4-yl]-2-methoxyquinoline-4-carboxamide

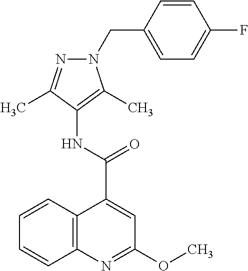 [SMILES: COc1cc(C(=O)Nc2c(C)nn(Cc3ccc(F)cc3)c2C)c2ccccc2n1]

In analogy to example 1), 150 mg (0.68 mmol) 1-(4-fluorobenzyl)-3,5-dimethyl-1H-pyrazol-4-amine (intermediate 1C) was stirred with 167 mg (0.82 mmol) 2-methoxyquinoline-4-carboxylic acid, 179 μL (1.03 mmol) N,N-diisopropylethylamine and 329 mg (1.03 mmol) TBTU in 5 mL tetrahydrofuran for 2 h at 25° C. The reaction mixture was evaporated and the residue partitioned between ethyl acetate and water. The layers were separated and the aqueous layer was extracted two further times with ethyl acetate. The combined organic layers were washed with brine, dried over sodium sulfate, filtered and evaporated. The residue was dissolved in dichloromethane and under evaporation adsorbed on Isolute® HM-N (Biotage). The isolute was given on a Biotage SNAP cartridge (25 g; KP-Sil) preequilibrated with hexane and purified via column chromatography on silica gel (solvent: hexane/0-100% ethyl acetate). The resulting crude product was again purified via preparative HPLC (method 3) to obtain 228 mg (0.56 mmol, 82%) of the desired title compound.

1H NMR (300 MHz, DMSO d₆): δ (ppm)=2.13 (s, 3H), 2.17 (s, 3H), 4.04 (s, 3H), 5.24 (s, 2H), 7.11-7.30 (m, 5H), 7.44-7.56 (m, 1H), 7.66-7.78 (m, 1H), 7.82-7.91 (m, 1H), 8.00-8.10 (m, 1H), 9.91 (s, 1H).

### Example 28

### 6,8-dichloro-N-[3,5-dimethyl-1-(3-methylbenzyl)-1H-pyrazol-4-yl]-2-(trifluoromethyl)quinoline-4-carboxamide

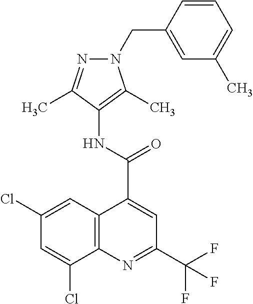 [SMILES: Cc1cccc(Cn2nc(C)c(NC(=O)c3cc(C(F)(F)F)nc4c(Cl)cc(Cl)cc34)c2C)c1]

In analogy to example 1), 50 mg (0.23 mmol) 3,5-dimethyl-1-(3-methylbenzyl)-1H-pyrazol-4-amine (intermediate 14C) was stirred with 86 mg (0.28 mmol) 6,8-dichloro-2-(trifluoromethyl)quinoline-4-carboxylic acid (intermediate 11A), 61 μL (0.35 mmol) N,N-diisopropylethylamine and 112 mg (0.35 mmol) TBTU in 5 mL tetrahydrofuran for 24 h at 25° C. to obtain 95 mg (0.19 mmol, 81%) of the desired title compound after preparative HPLC (method 3).

1H NMR (300 MHz, DMSO d₆): δ (ppm)=2.15 (s, 3H), 2.19 (s, 3H), 2.29 (s, 3H), 5.21 (s, 2H), 6.95 (d, 1H), 7.03 (s, 1H), 7.10 (d, 1H), 7.24 (t, 1H), 8.30 (d, 1H), 8.37-8.43 (m, 2H), 10.19 (s, 1H).

### Example 29

### 7-fluoro-N-[1-(4-fluorobenzyl)-3,5-dimethyl-1H-pyrazol-4-yl]-2-(trifluoromethyl)quinoline-4-carboxamide

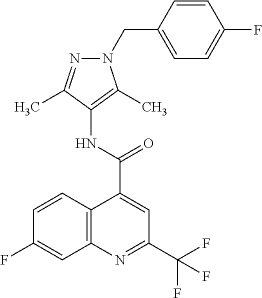 [SMILES: Cc1nn(Cc2ccc(F)cc2)c(C)c1NC(=O)c1cc(C(F)(F)F)nc2cc(F)ccc12]

In analogy to example 1), 50 mg (0.23 mmol) 1-(4-fluorobenzyl)-3,5-dimethyl-1H-pyrazol-4-amine (intermediate 1C) was stirred with 71 mg (0.27 mmol) 7-fluoro-2-(trifluoromethyl)quinoline-4-carboxylic acid (intermediate 17A), 60 μL (0.34 mmol) N,N-diisopropylethylamine and 109 mg (0.34 mmol) TBTU in 3 mL tetrahydrofuran for 24 h at 25° C. to obtain 93 mg (0.20 mmol, 89%) of the desired title compound after preparative HPLC (method 3).

1H NMR (300 MHz, DMSO d₆): δ (ppm)=2.15 (s, 3H), 2.19 (s, 3H), 5.25 (s, 2H), 7.10-7.29 (m, 4H), 7.87 (td, 1H), 8.09 (dd, 1H), 8.20 (s, 1H), 8.38 (dd, 1H), 10.12 (s, 1H).

### Example 30

### 6-fluoro-N-[1-(4-fluorobenzyl)-3,5-dimethyl-1H-pyrazol-4-yl]-2-(trifluoromethyl)quinoline-4-carboxamide

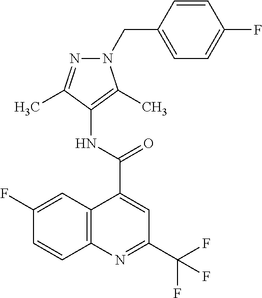 [SMILES: Cc1nn(Cc2ccc(F)cc2)c(C)c1NC(=O)c1cc(C(F)(F)F)nc2ccc(F)cc12]

In analogy to example 1), 50 mg (0.23 mmol) 1-(4-fluorobenzyl)-3,5-dimethyl-1H-pyrazol-4-amine (intermediate 1C) was stirred with 71 mg (0.27 mmol) 6-fluoro-2-(trifluoromethyl)quinoline-4-carboxylic acid (intermediate 18A), 60 μL (0.34 mmol) N,N-diisopropylethylamine and 110 mg (0.34 mmol) TBTU in 3 mL tetrahydrofuran for 3 h at 25° C. to obtain 95 mg (0.19 mmol, 81%) of the desired title compound after preparative HPLC (method 3).

1H NMR (300 MHz, CDCl₃): δ (ppm)=2.23 (s, 3H), 2.31 (s, 3H), 5.25 (s, 2H), 6.99-7.10 (m, 2H), 7.15 (dd, 3H), 7.62-7.74 (m, 1H), 7.93 (s, 1H), 8.09 (dd, 1H), 8.33 (dd, 1H).

### Example 31

### 6,8-dichloro-N-[3,5-dimethyl-1-(4-methylbenzyl)-1H-pyrazol-4-yl]-2-(trifluoromethyl)quinoline-4-carboxamide

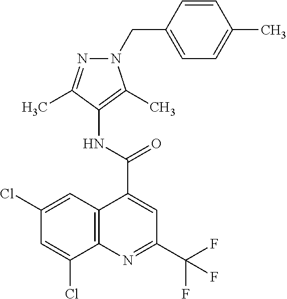 [SMILES: Cc1ccc(Cn2nc(C)c(NC(=O)c3cc(C(F)(F)F)nc4c(Cl)cc(Cl)cc34)c2C)cc1]

In analogy to example 1), 50 mg (0.23 mmol) 3,5-dimethyl-1-(4-methylbenzyl)-1H-pyrazol-4-amine (intermediate 13C) was stirred with 86 mg (0.29 mmol) 6,8-dichloro-2-(trifluoromethyl)quinoline-4-carboxylic acid (intermediate 11A), 61 μL (0.35 mmol) N,N-diisopropylethylamine and 112 mg (0.35 mmol) TBTU in 5 mL tetrahydrofuran for 24 h at 25° C. The reaction mixture was evaporated, dissolved in dichloromethane and under evaporation adsorbed on Isolute® HM-N (Biotage). The isolute was given on a Biotage SNAP cartridge (25 g; KP-Sil) preequilibrated with hexane and purified via column chromatography on silica gel (solvent: hexane/0-40% ethyl acetate) to obtain 50 mg (0.09 mmol, 42%) of the desired title compound.

1H NMR (300 MHz, DMSO ds): δ (ppm)=2.14 (s, 3H), 2.17 (s, 3H), 2.28 (s, 3H), 5.20 (s, 2H), 7.07 (m, 2H), 7.16 (m, 2H), 8.29 (d, 1H), 8.37-8.45 (m, 2H), 10.20 (s, 1H).

### Example 32

### 6-bromo-N-[1-(4-fluorobenzyl)-5-methyl-3-(trifluoromethyl)-1H-pyrazol-4-yl]-2-(trifluoromethyl)quinoline-4-carboxamide

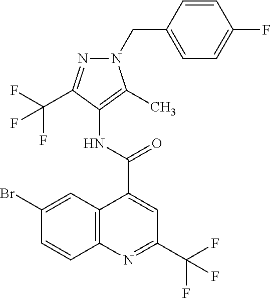 [SMILES: Cc1c(NC(=O)c2cc(C(F)(F)F)nc3ccc(Br)cc23)c(C(F)(F)F)nn1Cc1ccc(F)cc1]

In analogy to example 1), 75 mg (0.20 mmol, 74%) 1-(4-fluorobenzyl)-5-methyl-3-(trifluoromethyl)-1H-pyrazol-4-amine (intermediate 25C) was stirred with 78 mg (0.24 mmol) 6-bromo-2-(trifluoromethyl)quinoline-4-carboxylic acid (intermediate 1A), 53 μL (0.30 mmol) N,N-diisopropylethylamine and 98 mg (0.30 mmol) TBTU in 3 mL tetrahydrofuran for 24 h at 25° C. to obtain 62 mg (0.10 mmol, 49%) of the desired title compound after preparative HPLC (method 3).

1H NMR (300 MHz, DMSO d₆): δ (ppm)=2.31 (s, 3H), 5.47 (s, 2H), 7.19-7.36 (m, 4H), 8.11-8.20 (m, 1H), 8.20-8.30 (m, 2H), 8.42 (d, 1H), 10.52 (s, 1H).

### Example 33

### N-(1-benzyl-3,5-dimethyl-1H-pyrazol-4-yl)-6-bromo-2-(trifluoromethyl)quinoline-4-carboxamide

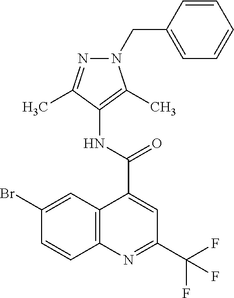 [SMILES: Cc1nn(Cc2ccccc2)c(C)c1NC(=O)c1cc(C(F)(F)F)nc2ccc(Br)cc12]

In analogy to example 1), 50 mg (0.25 mmol) 1-benzyl-3,5-dimethyl-1H-pyrazol-4-amine (intermediate 30C) was stirred with 95 mg (0.30 mmol) 6-bromo-2-(trifluoromethyl)quinoline-4-carboxylic acid (intermediate 1A), 65 μL (0.37 mmol) N,N-diisopropylethylamine and 120 mg (0.37 mmol) TBTU in 5 mL tetrahydrofuran for 24 h at 25° C. to obtain 72 mg (0.14 mmol, 57%) of the desired title compound after preparative HPLC (method 3).

1H NMR (400 MHz, DMSO d₆): δ (ppm)=2.15 (s, 3H), 2.19 (s, 3H), 5.26 (s, 2H), 7.16-7.21 (m, 2H), 7.26-7.32 (m, 1H), 7.33-7.39 (m, 2H), 8.12-8.17 (m, 1H), 8.23 (d, 1H), 8.29 (s, 1H), 8.51 (d, 1H), 10.16 (s, 1H).

### Example 34

### 5-bromo-N-[1-(4-fluorobenzyl)-3,5-dimethyl-1H-pyrazol-4-yl]-2-(trifluoromethyl)quinoline-4-carboxamide

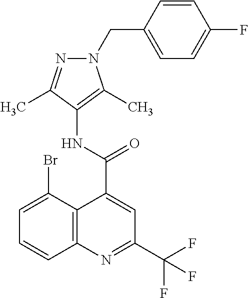 [SMILES: Cc1nn(Cc2ccc(F)cc2)c(C)c1NC(=O)c1cc(C(F)(F)F)nc2cccc(Br)c12]

In analogy to example 1), 50 mg (0.23 mmol) 1-(4-fluorobenzyl)-3,5-dimethyl-1H-pyrazol-4-amine (intermediate 1C) was stirred with 88 mg (0.27 mmol) 5-bromo-2-(trifluoromethyl)quinoline-4-carboxylic acid (intermediate 33A), 60 μL (0.34 mmol) N,N-diisopropylethylamine and 109 mg (0.34 mmol) TBTU in 5 mL tetrahydrofuran for 24 h at 25° C. to obtain 48 mg (0.09 mmol, 40%) of the desired title compound after preparative HPLC (method 3).

1H NMR (300 MHz, DMSO d₆): δ (ppm)=2.18 (s, 3H), 2.23 (s, 3H), 5.22 (s, 2H), 7.13-7.29 (m, 4H), 7.86-7.94 (m, 1H), 8.12 (s, 1H), 8.25 (dd, 1H), 8.33 (dd, 1H), 10.07 (s, 1H).

### Example 35

### 6,8-dichloro-N-[1-(3,4-difluorobenzyl)-3,5-dimethyl-1H-pyrazol-4-yl]-2-(trifluoromethyl)quinoline-4-carboxamide

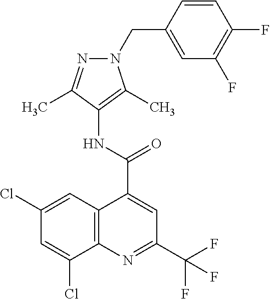 [SMILES: Cc1nn(Cc2ccc(F)c(F)c2)c(C)c1NC(=O)c1cc(C(F)(F)F)nc2c(Cl)cc(Cl)cc12]

In analogy to example 1), 50 mg (0.21 mmol) 1-(3,4-difluorobenzyl)-3,5-dimethyl-1H-pyrazol-4-amine (intermediate 4C) was stirred with 78 mg (0.25 mmol) 6,8-dichloro-2-(trifluoromethyl)quinoline-4-carboxylic acid (intermediate 11A), 55 μL (0.32 mmol) N,N-diisopropylethylamine and 102 mg (0.32 mmol) TBTU in 5 mL tetrahydrofuran for 24 h at 25° C. to obtain 35 mg (0.05 mmol, 28%) of the desired title compound after preparative HPLC (method 3).

1H NMR (300 MHz, DMSO d₆): δ (ppm)=2.15 (s, 3H), 2.20 (s, 3H), 5.26 (s, 2H), 7.02 (br. s., 1H), 7.20 (d, 1H), 7.37-7.51 (m, 1H), 8.30 (d, 1H), 8.37-8.44 (m, 2H), 10.21 (s, 1H).

### Example 36

### 2-bromo-N-[1-(4-fluorobenzyl)-3,5-dimethyl-1H-pyrazol-4-yl]quinoline-4-carboxamide

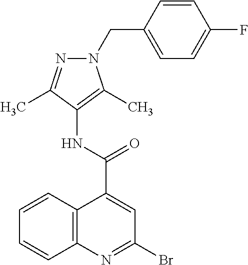 [SMILES: Cc1nn(Cc2ccc(F)cc2)c(C)c1NC(=O)c1cc(Br)nc2ccccc12]

In analogy to example 1), 750 mg (3.42 mmol) 1-(4-fluorobenzyl)-3,5-dimethyl-1H-pyrazol-4-amine (intermediate 1C) was stirred with 1.03 g (4.10 mmol) 2-bromoquinoline-4-carboxylic acid, 894 μL (5.13 mmol) N,N-diisopropylethylamine and 1.65 g (5.13 mmol) TBTU in 5 mL tetrahydrofuran for 2 h at 25° C. The reaction mixture was evaporated and the residue partitioned between ethyl acetate and water. The layers were separated and the aqueous layer was extracted two further times with ethyl acetate. The combined organic layers were washed with brine, dried over sodium sulfate, filtered and evaporated. The residue was dissolved in dichloromethane and under evaporation adsorbed on Isolute® HM-N (Biotage). The isolute was given on a Biotage SNAP cartridge (100 g; KP-Sil) preequilibrated with hexane and purified via column chromatography on silica gel (solvent: hexane/0-100% ethyl acetate) to obtain 1.47 g (3.24 mmol, 95%) of the desired title compound

1H NMR (400 MHz, DMSO d₆): δ (ppm)=2.14 (s, 3H), 2.18 (s, 3H), 5.24 (s, 2H), 7.15-7.27 (m, 4H), 7.77 (ddd, 1H), 7.90 (ddd, 1H), 7.94 (s, 1H), 8.06 (d, 1H), 8.16 (dd, 1H), 10.02 (s, 1H).

### Example 37

### 7-bromo-N-[1-(4-fluorobenzyl)-3,5-dimethyl-1H-pyrazol-4-yl]-1,2,3,4-tetrahydroacridine-9-carboxamide

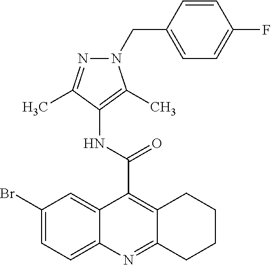 [SMILES: Cc1nn(Cc2ccc(F)cc2)c(C)c1NC(=O)c1c2c(nc3ccc(Br)cc13)CCCC2]

50 mg (0.23 mmol) 1-(4-fluorobenzyl)-3,5-dimethyl-1H-pyrazol-4-amine (intermediate 1C) was stirred with 105 mg (0.34 mmol) 7-bromo-1,2,3,4-tetrahydroacridine-9-carboxylic acid, 60 μL (0.34 mmol) N,N-diisopropylethylamine, 52 mg (0.34 mmol) HOBt and 219 mg (1.14 mmol) EDC in 5 mL N,N-dimethylformamide for 24 h at 25° C. The reaction mixture was partitioned between dichloromethane and water. The organic layer was washed with brine, dried over sodium sulfate, filtered and evaporated. The residue purified via preparative HPLC (method 3) to obtain 59 mg (0.11 mmol, 51%) of the desired title compound.

1H NMR (300 MHz, CDCl₃): δ (ppm)=1.90-2.04 (m, 4H), 2.27 (s, 3H), 2.35 (s, 3H), 3.02-3.17 (m, 4H), 5.23 (s, 2H), 6.97-7.10 (m, 3H), 7.10-7.21 (m, 2H), 7.73 (dd, 1H), 7.87 (d, 1H), 8.05 (d, 1H).

### Example 38

### 6,8-dichloro-N-[1-(4-fluorobenzyl)-5-methyl-3-(trifluoromethyl)-1H-pyrazol-4-yl]-2-(trifluoromethyl)quinoline-4-carboxamide

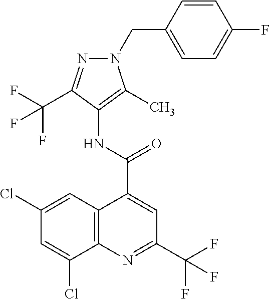 [SMILES: Cc1c(NC(=O)c2cc(C(F)(F)F)nc3c(Cl)cc(Cl)cc23)c(C(F)(F)F)nn1Cc1ccc(F)cc1]

In analogy to example 1), 75 mg (0.20 mmol, 74%) 1-(4-fluorobenzyl)-5-methyl-3-(trifluoromethyl)-1H-pyrazol-4-amine (intermediate 25C) was stirred with 76 mg (0.24 mmol) 6,8-dichloro-2-(trifluoromethyl)quinoline-4-carboxylic acid (intermediate 11A), 53 μL (0.30 mmol) N,N-diisopropylethylamine and 98 mg (0.30 mmol) TBTU in 5 mL tetrahydrofuran for 24 h at 25° C. to obtain 71 mg (0.12 mmol, 61%) of the desired title compound after preparative HPLC (method 3).

1H NMR (300 MHz, DMSO d₆): δ (ppm)=2.31 (s, 3H), 5.47 (s, 2H), 7.18-7.37 (m, 4H), 8.20 (d, 1H), 8.36-8.45 (m, 2H), 10.56 (s, 1H).

### Example 39

### 6,7-difluoro-N-[1-(4-fluorobenzyl)-3,5-dimethyl-1H-pyrazol-4-yl]-2-(trifluoromethyl)quinoline-4-carboxamide

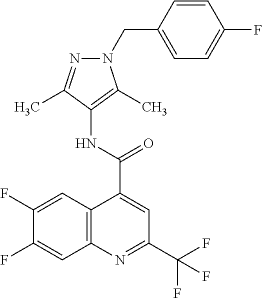 [SMILES: Cc1nn(Cc2ccc(F)cc2)c(C)c1NC(=O)c1cc(C(F)(F)F)nc2cc(F)c(F)cc12]

In analogy to example 1), 50 mg (0.23 mmol) 1-(4-fluorobenzyl)-3,5-dimethyl-1H-pyrazol-4-amine (intermediate 1C) was stirred with 76 mg (0.27 mmol) 6,7-difluoro-2-(trifluoromethyl)quinoline-4-carboxylic acid (intermediate 19A), 60 μL (0.34 mmol) N,N-diisopropylethylamine and 110 mg (0.34 mmol) TBTU in 5 mL tetrahydrofuran for 24 h at 25° C. to obtain 50 mg (0.11 mmol, 46%) of the desired title compound after preparative HPLC (method 3).

1H NMR (300 MHz, DMSO d₆): δ (ppm)=2.14 (s, 3H), 2.18 (s, 3H), 5.25 (s, 2H), 7.14-7.28 (m, 4H), 8.26 (dd, 1H), 8.31 (s, 1H), 8.42 (dd, 1H), 10.19 (s, 1H).

### Example 40

### 2-cyclopropyl-8-fluoro-N-[1-(4-fluorobenzyl)-3,5-dimethyl-1H-pyrazol-4-yl]quinoline-4-carboxamide

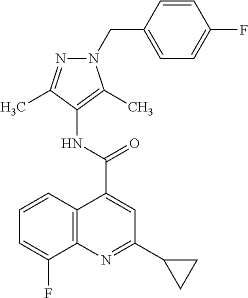 [SMILES: Cc1nn(Cc2ccc(F)cc2)c(C)c1NC(=O)c1cc(C2CC2)nc2c(F)cccc12]

In analogy to example 1), 75 mg (0.34 mmol) 1-(4-fluorobenzyl)-3,5-dimethyl-1H-pyrazol-4-amine (intermediate 1C) was stirred with 95 mg (0.41 mmol) 2-cyclopropyl-8-fluoroquinoline-4-carboxylic acid (intermediate 28A), 89 μL (0.51 mmol) N,N-diisopropylethylamine and 165 mg (0.51 mmol) TBTU in 5 mL tetrahydrofuran for 24 h at 25° C. to obtain 103 mg (0.23 mmol, 68%) of the desired title compound after preparative HPLC (method 3).

1H NMR (400 MHz, DMSO d₆): δ (ppm)=1.10-1.20 (m, 4H), 2.14 (s, 3H), 2.18 (s, 3H), 2.39-2.48 (m, 1H), 5.24 (s, 2H), 7.10-7.30 (m, 4H), 7.48-7.64 (m, 2H), 7.72 (s, 1H), 7.84-7.94 (m, 1H), 9.91 (s, 1H).

### Example 41

### 6-bromo-N-[1-(2,4-difluorobenzyl)-3,5-dimethyl-1H-pyrazol-4-yl]-2-(trifluoromethyl)quinoline-4-carboxamide

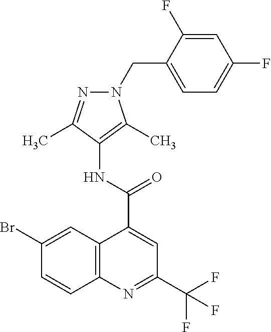 [SMILES: Cc1nn(Cc2ccc(F)cc2F)c(C)c1NC(=O)c1cc(C(F)(F)F)nc2ccc(Br)cc12]

50 mg (0.21 mmol) 1-(2,4-difluorobenzyl)-3,5-dimethyl-1H-pyrazol-4-amine (intermediate 5C) was dissolved in 5 mL tetrahydrofuran and 81 mg (0.25 mmol) 2 6-bromo-2-(trifluoromethyl)quinoline-4-carboxylic acid (intermediate 1A), 55 μL (0.32 mmol) N,N-diisopropylethylamine and 164 mg (0.32 mmol) PyBOP in 5 mL tetrahydrofuran were added. The reaction mixture was stirred for 24 h at 25° C. After evaporation the residue was dissolved in 2.5 mL dimethylformamide and purified via preparative HPLC (method 3) to obtain 44 mg (0.08 mmol, 37%) of the desired title compound after preparative HPLC (method 3).

1H NMR (300 MHz, DMSO d₆): δ (ppm)=2.13 (s, 3H), 2.24 (s, 3H), 5.27 (s, 2H), 7.05-7.23 (m, 2H), 7.24-7.35 (m, 1H), 8.15 (dd, 1H), 8.23 (d, 1H), 8.30 (s, 1H), 8.50 (d, 1H), 10.19 (s, 1H).

### Example 42

### N-[1-(2, 6-difluorobenzyl)-3,5-dimethyl-1H-pyrazol-4-yl]-2-methoxyquinoline-4-carboxamide

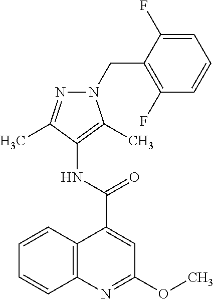 [SMILES: COc1cc(C(=O)Nc2c(C)nn(Cc3c(F)cccc3F)c2C)c2ccccc2n1]

In analogy to example 1), 50 mg (0.21 mmol) 1-(2,6-difluorobenzyl)-3,5-dimethyl-1H-pyrazol-4-amine (intermediate 6C) was stirred with 51 mg (0.25 mmol) 2-methoxyquinoline-4-carboxylic acid, 55 μL (0.32 mmol) N,N-diisopropylethylamine and 102 mg (0.32 mmol) TBTU in 5 mL tetrahydrofuran for 24 h at 25° C. to obtain 59 mg (0.13 mmol, 62%) of the desired title compound after preparative HPLC (method 3).

1H NMR (300 MHz, DMSO d₆): δ (ppm)=2.04 (s, 3H), 2.30 (s, 3H), 4.04 (s, 3H), 5.23 (s, 2H), 7.15 (t, 2H), 7.22 (s, 1H), 7.40-7.56 (m, 2H), 7.69-7.77 (m, 1H), 7.86 (d, 1H), 8.05 (d, 1H), 9.88 (s, 1H).

### Example 43

### 6-bromo-N-[1-(4-fluorobenzyl)-3,5-dimethyl-1H-pyrazol-4-yl]-2-methylquinoline-4-carboxamide

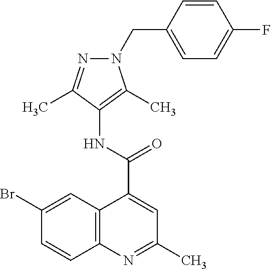 [SMILES: Cc1cc(C(=O)Nc2c(C)nn(Cc3ccc(F)cc3)c2C)c2cc(Br)ccc2n1]

In analogy to example 1), 750 mg (3.42 mmol) 1-(4-fluorobenzyl)-3,5-dimethyl-1H-pyrazol-4-amine (intermediate 1C) was stirred with 1.09 g (4.10 mmol) 6-bromo-2-methylquinoline-4-carboxylic acid, 894 μL (5.13 mmol) N,N-diisopropylethylamine and 1.65 g (5.13 mmol) TBTU in 20 mL tetrahydrofuran for 2 h at 25° C. The reaction mixture was evaporated and the residue partitioned between ethyl acetate and water. The layers were separated and the aqueous layer was extracted two further times with ethyl acetate. The combined organic layers were washed with brine, dried over sodium sulfate, filtered and evaporated. The residue was dissolved in dichloromethane and under evaporation adsorbed on Isolute® HM-N (Biotage). The isolute was given on a Biotage SNAP cartridge (100 g; KP-Sil) preequilibrated with hexane and purified via column chromatography on silica gel (solvent: hexane/0-100% ethyl acetate) to obtain 847 mg (1.81 mmol, 53%) of the desired title compound

1H NMR (400 MHz, DMSO d₆): δ (ppm)=2.13 (s, 3H), 2.17 (s, 3H), 2.72 (s, 3H), 5.24 (s, 2H), 7.16-7.27 (m, 4H), 7.72 (s, 1H), 7.89-7.93 (m, 1H), 7.94-7.98 (m, 1H), 8.32 (d, 1H), 9.96 (s, 1H).

### Example 44

### N-[1-(4-cyanobenzyl)-3,5-dimethyl-1H-pyrazol-4-yl]-2,6-dimethylquinoline-4-carboxamide

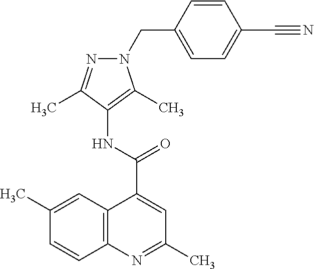 [SMILES: Cc1ccc2nc(C)cc(C(=O)Nc3c(C)nn(Cc4ccc(C#N)cc4)c3C)c2c1]

In analogy to example 1), 50 mg (0.22 mmol) 4-[(4-amino-3,5-dimethyl-1H-pyrazol-1-yl)methyl]benzonitrile (intermediate 8C) was stirred with 53 mg (0.27 mmol) 2,6-dimethylquinoline-4-carboxylic acid, 58 μL (0.33 mmol) N,N-diisopropylethylamine and 106 mg (0.33 mmol) TBTU in 5 mL tetrahydrofuran for 24 h at 25° C. to obtain 32 mg (0.08 mmol, 35%) of the desired title compound after preparative HPLC (method 3).

1H NMR (300 MHz, DMSO d₆): δ (ppm)=2.15 (s, 3H), 2.17 (s, 3H), 2.69 (s, 3H), 5.37 (s, 2H), 7.33 (d, 2H), 7.57 (s, 1H), 7.61 (dd, 1H), 7.82-7.92 (m, 4H), 9.87 (s, 1H).

### Example 45

### N-[3,5-dimethyl-1-(4-methylbenzyl)-1H-pyrazol-4-yl]-2,6-dimethylquinoline-4-carboxamide

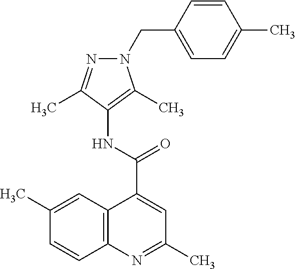

In analogy to example 1), 50 mg (0.23 mmol) 3,5-dimethyl-1-(4-methylbenzyl)-1H-pyrazol-4-amine (intermediate 13C) was stirred with 56 mg (0.28 mmol) 2,6-dimethylquinoline-4-carboxylic acid, 60 μL (0.35 mmol) N,N-diisopropylethylamine and 112 mg (0.35 mmol) TBTU in 5 mL tetrahydrofuran for 24 h at 25° C. to obtain 48 mg (0.12 mmol, 51%) of the desired title compound after preparative HPLC (method 3).

1H NMR (400 MHz, DMSO d₆): δ (ppm)=2.14 (s, 3H), 2.16 (s, 3H), 2.28 (s, 3H), 2.48 (s, 3H), 2.69 (s, 3H), 5.19 (s, 2H), 7.08 (d, 2H), 7.16 (d, 2H), 7.56 (s, 1H), 7.61 (dd, 1H), 7.85-7.91 (m, 2H), 9.83 (s, 1H).

### Example 46

### 2-cyclopropyl-5-fluoro-N-[1-(4-fluorobenzyl)-3,5-dimethyl-1H-pyrazol-4-yl]quinoline-4-carboxamide

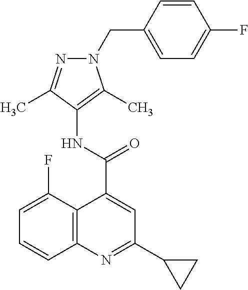 [SMILES: Cc1nn(Cc2ccc(F)cc2)c(C)c1NC(=O)c1cc(C2CC2)nc2cccc(F)c12]

In analogy to example 1), 75 mg (0.34 mmol) 1-(4-fluorobenzyl)-3,5-dimethyl-1H-pyrazol-4-amine (intermediate 1C) was stirred with 95 mg (0.41 mmol) 2-cyclopropyl-5-fluoroquinoline-4-carboxylic acid (intermediate 25A), 89 μL (0.51 mmol) N,N-diisopropylethylamine and 165 mg (0.51 mmol) TBTU in 5 mL tetrahydrofuran for 24 h at 25° C. to obtain 75 mg (0.17 mmol, 51%) of the desired title compound after preparative HPLC (method 3).

1H NMR (400 MHz, DMSO d₆): δ (ppm)=1.10-1.21 (m, 4H), 2.14 (s, 3H), 2.18 (s, 3H), 2.40-2.48 (m, 1H), 5.24 (s, 2H), 7.13-7.27 (m, 4H), 7.49-7.62 (m, 2H), 7.72 (s, 1H), 7.84-7.93 (m, 1H), 9.91 (s, 1H).

### Example 47

### 6-bromo-N-[1-(3,4-difluorobenzyl)-3,5-dimethyl-1H-pyrazol-4-yl]-2-(trifluoromethyl)quinoline-4-carboxamide

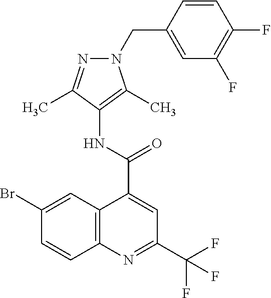 [SMILES: Cc1nn(Cc2ccc(F)c(F)c2)c(C)c1NC(=O)c1cc(C(F)(F)F)nc2ccc(Br)cc12]

In analogy to example 41), 50 mg (0.22 mmol) 1-(3,4-difluorobenzyl)-3,5-dimethyl-1H-pyrazol-4-amine (intermediate 4C) was stirred with 81 mg (0.25 mmol) 6-bromo-2-(trifluoromethyl)quinoline-4-carboxylic acid (intermediate 1A), 55 μL (0.32 mmol) N,N-diisopropylethylamine and 165 mg (0.32 mmol) PyBOP in 5 mL tetrahydrofuran for 24 h at 25° C. to obtain 25 mg (0.04 mmol, 21%) of the desired title compound after preparative HPLC (method 3).

1H NMR (300 MHz, DMSO d₆): δ (ppm)=2.16 (s, 3H), 2.20 (s, 3H), 5.26 (s, 2H), 7.03 (dd, 1H), 7.17-7.29 (m, 1H), 7.44 (dt, 1H), 8.15 (dd, 1H), 8.23 (d, 1H), 8.30 (s, 1H), 8.50 (d, 1H), 10.19 (s, 1H).

### Example 48

### 6,8-dichloro-N-[1-(2,4-difluorobenzyl)-3,5-dimethyl-1H-pyrazol-4-yl]-2-(trifluoromethyl)quinoline-4-carboxamide

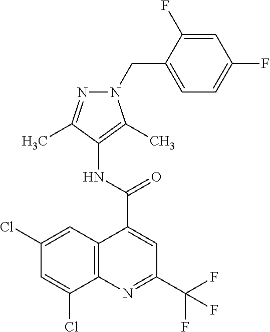 [SMILES: Cc1nn(Cc2ccc(F)cc2F)c(C)c1NC(=O)c1cc(C(F)(F)F)nc2c(Cl)cc(Cl)cc12]

In analogy to example 1), 50 mg (0.22 mmol) 1-(2,4-difluorobenzyl)-3,5-dimethyl-1H-pyrazol-4-amine (intermediate 5C) was stirred with 78 mg (0.25 mmol) 6,8-dichloro-2-(trifluoromethyl)quinoline-4-carboxylic acid (intermediate 11A), 53 μL (0.32 mmol) N,N-diisopropylethylamine and 102 mg (0.32 mmol) TBTU in 5 mL tetrahydrofuran for 24 h at 25° C. to obtain 48 mg (0.09 mmol, 41%) of the desired title compound after preparative HPLC (method 3).

1H NMR (300 MHz, DMSO d₆): δ (ppm)=2.11 (d, 3H), 2.23 (d, 3H), 5.25 (d, 2H), 7.11 (br. s., 2H), 7.28 (d, 1H), 8.28 (d, 1H), 8.42 (d, 2H), 10.22 (d, 1H).

### Example 49

### 6-bromo-N-[3,5-dimethyl-1-(2,4,6-trifluorobenzyl)-1H-pyrazol-4-yl]-2-(trifluoromethyl)quinoline-4-carboxamide

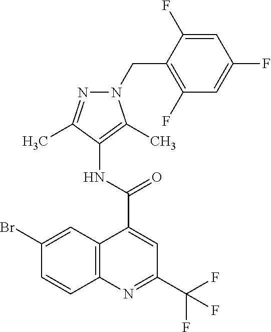 [SMILES: Cc1nn(Cc2c(F)cc(F)cc2F)c(C)c1NC(=O)c1cc(C(F)(F)F)nc2ccc(Br)cc12]

In analogy to example 41), 50 mg (0.20 mmol) 3,5-dimethyl-1-(2,4,6-trifluorobenzyl)-1H-pyrazol-4-amine (intermediate 7C) was stirred with 75 mg (0.24 mmol) 6-bromo-2-(trifluoromethyl)quinoline-4-carboxylic acid (intermediate 1A), 51 μL (0.29 mmol) N,N-diisopropylethylamine and 153 mg (0.29 mmol) PyBOP in 5 mL tetrahydrofuran for 24 h at 25° C. to obtain 71 mg (0.11 mmol, 59%) of the desired title compound after preparative HPLC (method 3).

1H NMR (300 MHz, DMSO d₆): δ (ppm)=2.07 (s, 3H), 2.31 (s, 3H), 5.16-5.32 (m, 2H), 7.26 (t, 2H), 8.15 (dd, 1H), 8.23 (d, 1H), 8.30 (s, 1H), 8.46-8.54 (m, 1H), 10.16 (s, 1H).

### Example 50

### N-[3,5-dimethyl-1-(2,4,6-trifluorobenzyl)-1H-pyrazol-4-yl]-2-methoxyquinoline-4-carboxamide

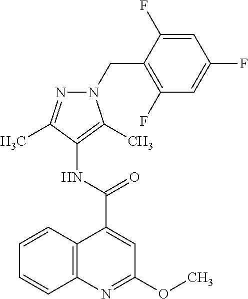 [SMILES: COc1cc(C(=O)Nc2c(C)nn(Cc3c(F)cc(F)cc3F)c2C)c2ccccc2n1]

In analogy to example 1), 50 mg (0.20 mmol) 3,5-dimethyl-1-(2,4,6-trifluorobenzyl)-1H-pyrazol-4-amine (intermediate 7C) was stirred with 48 mg (0.24 mmol) 2-methoxyquinoline-4-carboxylic acid, 51 μL (0.29 mmol) N,N-diisopropylethylamine and 165 mg (0.51 mmol) TBTU in 5 mL tetrahydrofuran for 24 h at 25° C. to obtain 6 mg (0.01 mmol, 7%) of the desired title compound after preparative HPLC (method 5d).

1H NMR (400 MHz, DMSO d₆): δ (ppm)=2.05 (s, 2H), 2.11 (s, 1H), 2.21 (s, 1H), 2.30 (s, 2H), 4.04 (s, 3H), 5.19 (s, 2H), 7.19-7.31 (m, 3H), 7.51 (td, 1H), 7.73 (td, 1H), 7.86 (d, 1H), 8.05 (d, 1H), 9.77-9.93 (m, 1H).

### Example 51

### N-[3,5-dimethyl-1-(2-methylbenzyl)-1H-pyrazol-4-yl]-2-methoxyquinoline-4-carboxamide

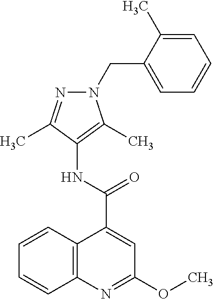 [SMILES: COc1cc(C(=O)Nc2c(C)nn(Cc3ccccc3C)c2C)c2ccccc2n1]

In analogy to example 1), 50 mg (0.23 mmol) 3,5-dimethyl-1-(2-methylbenzyl)-1H-pyrazol-4-amine (intermediate 15C) was stirred with 56 mg (0.28 mmol) 2-methoxyquinoline-4-carboxylic acid, 61 μL (0.35 mmol) N,N-diisopropylethylamine and 112 mg (0.35 mmol) TBTU in 5 mL tetrahydrofuran for 24 h at 25° C. to obtain 43 mg (0.10 mmol, 44%) of the desired title compound after preparative HPLC (method 3).

1H NMR (300 MHz, DMSO d₆): δ (ppm)=2.14 (s, 6H), 2.35 (s, 3H), 4.05 (s, 3H), 5.25 (s, 2H), 6.60 (d, 1H), 7.09-7.26 (m, 4H), 7.52 (td, 1H), 7.74 (td, 1H), 7.83-7.90 (m, 1H), 8.07 (d, 1H), 9.91 (s, 1H).

### Example 52

### 6-bromo-N-[3,5-dimethyl-1-(3-methylbenzyl)-1H-pyrazol-4-yl]-2-(trifluoromethyl)quinoline-4-carboxamide

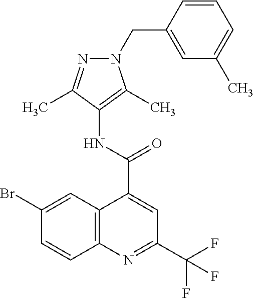 [SMILES: Cc1cccc(Cn2nc(C)c(NC(=O)c3cc(C(F)(F)F)nc4ccc(Br)cc34)c2C)c1]

In analogy to example 1), 50 mg (0.23 mmol) 3,5-dimethyl-1-(3-methylbenzyl)-1H-pyrazol-4-amine (intermediate 14C) was stirred with 89 mg (0.28 mmol) 6-bromo-2-(trifluoromethyl)quinoline-4-carboxylic acid (intermediate 1A), 61 μL (0.35 mmol) N,N-diisopropylethylamine and 112 mg (0.35 mmol) TBTU in 5 mL tetrahydrofuran for 24 h at 25° C. to obtain 84 mg (0.16 mmol, 69%) of the desired title compound after preparative HPLC (method 3).

1H NMR (300 MHz, DMSO d₆): δ (ppm)=2.15 (s, 3H), 2.19 (s, 3H), 2.28 (s, 3H), 5.21 (s, 2H), 6.95 (d, 1H), 7.03 (s, 1H), 7.10 (d, 1H), 7.24 (t, 1H), 8.11-8.18 (m, 1H), 8.23 (d, 1H), 8.30 (s, 1H), 8.50 (d, 1H), 10.18 (s, 1H).

### Example 53

### 8-bromo-N-[1-(4-fluorobenzyl)-3,5-dimethyl-1H-pyrazol-4-yl]-2-methylquinoline-4-carboxamide

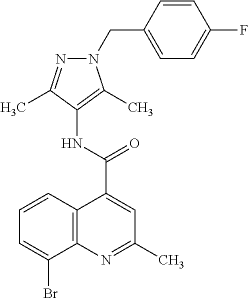 [SMILES: Cc1cc(C(=O)Nc2c(C)nn(Cc3ccc(F)cc3)c2C)c2cccc(Br)c2n1]

In analogy to example 1), 750 mg (3.42 mmol) 1-(4-fluorobenzyl)-3,5-dimethyl-1H-pyrazol-4-amine (intermediate 1C) was stirred with 1.09 g (4.10 mmol) 8-bromo-2-methylquinoline-4-carboxylic acid (intermediate 13A), 894 μL (5.13 mmol) N,N-diisopropylethylamine and 1.65 g (5.13 mmol) TBTU in 5 mL tetrahydrofuran for 2 h at 25° C. The reaction mixture was evaporated, the residue was dissolved in dichloromethane and under evaporation adsorbed on Isolute® HM-N (Biotage). The isolute was given on a Biotage SNAP cartridge (100 g; KP-Sil) preequilibrated with hexane and purified via column chromatography on silica gel (solvent: hexane/0-100% ethyl acetate) to obtain 1.38 g (2.95 mmol, 86%) of the desired title compound

1H NMR (300 MHz, DMSO d₆): δ (ppm)=2.14 (s, 3H), 2.18 (s, 3H), 2.78 (s, 3H), 5.24 (s, 2H), 7.10-7.29 (m, 4H), 7.53 (t, 1H), 7.73 (s, 1H), 8.05-8.23 (m, 2H), 9.93 (s, 1H).

### Example 54

### 6-bromo-N-[3,5-dimethyl-1-(4-methylbenzyl)-1H-pyrazol-4-yl]-2-(trifluoromethyl)quinoline-4-carboxamide

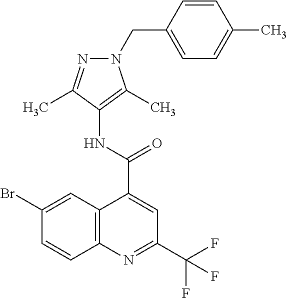 [SMILES: Cc1ccc(Cn2nc(C)c(NC(=O)c3cc(C(F)(F)F)nc4ccc(Br)cc34)c2C)cc1]

In analogy to example 1), 50 mg (0.23 mmol) 3,5-dimethyl-1-(4-methylbenzyl)-1H-pyrazol-4-amine (intermediate 13C) was stirred with 89 mg (0.28 mmol) 6-bromo-2-(trifluoromethyl)quinoline-4-carboxylic acid (intermediate 1A), 61 μL (0.35 mmol) N,N-diisopropylethylamine and 112 mg (0.35 mmol) TBTU in 5 mL tetrahydrofuran for 24 h at 25° C. The reaction mixture was evaporated and dissolved in acetonitrile. The precipitated product was isolated by filtration, washed with acetonitrile and dried in high vacuum to obtain 75 mg (0.14 mmol, 59%) of the desired title compound after preparative HPLC (method 3).

1H NMR (300 MHz, DMSO d₆): δ (ppm)=2.15 (s, 3H), 2.17 (s, 3H), 2.28 (s, 3H), 5.21 (s, 2H), 7.08 (m, 2H), 7.16 (m, 2H), 8.15 (dd, 1H), 8.23 (d, 1H), 8.29 (s, 1H), 8.50 (d, 1H), 10.16 (s, 1H).

### Example 55

### N-[1-(3,4-difluorobenzyl)-3,5-dimethyl-1H-pyrazol-4-yl]-2-methoxyquinoline-4-carboxamide

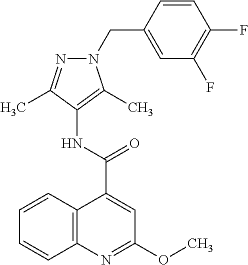 [SMILES: COc1cc(C(=O)Nc2c(C)nn(Cc3ccc(F)c(F)c3)c2C)c2ccccc2n1]

In analogy to example 1), 50 mg (0.21 mmol) 1-(3,4-difluorobenzyl)-3,5-dimethyl-1H-pyrazol-4-amine (intermediate 4C) was stirred with 51 mg (0.25 mmol) 2-methoxyquinoline-4-carboxylic acid, 55 μL (0.32 mmol) N,N-diisopropylethylamine and 102 mg (0.32 mmol) TBTU in 5 mL tetrahydrofuran for 24 h at 25° C. to obtain 19 mg (0.04 mmol, 20%) of the desired title compound after preparative HPLC (method 3).

1H NMR (400 MHz, DMSO d₆): δ (ppm)=2.14 (s, 3H), 2.16-2.20 (m, 3H), 4.04 (s, 3H), 5.25 (s, 2H), 7.02 (ddd, 1H), 7.18-7.27 (m, 2H), 7.43 (dt, 1H), 7.51 (ddd, 1H), 7.73 (ddd, 1H), 7.84-7.89 (m, 1H), 8.05 (dd, 1H), 9.90 (s, 1H).

### Example 56

### 6-bromo-N-{3,5-dimethyl-1-[4-(trifluoromethoxy)benzyl]-1H-pyrazol-4-yl}-2-(trifluoromethyl)quinoline-4-carboxamide

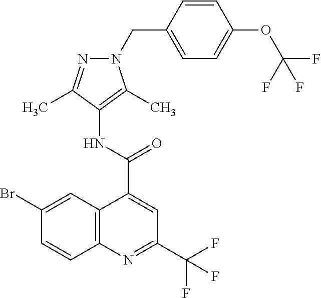 [SMILES: Cc1nn(Cc2ccc(OC(F)(F)F)cc2)c(C)c1NC(=O)c1cc(C(F)(F)F)nc2ccc(Br)cc12]

In analogy to example 1), 100 mg (0.18 mmol, 50%) 3,5-dimethyl-1-[4-(trifluoromethoxy)benzyl]-1H-pyrazol-4-amine (intermediate 19c) was stirred with 67 mg (0.21 mmol) 6-bromo-2-(trifluoromethyl)quinoline-4-carboxylic acid (intermediate 1A), 46 μL (0.26 mmol) N,N-diisopropylethylamine and 84 mg (0.26 mmol) TBTU in 3 mL tetrahydrofuran for 24 h at 25° C. to obtain 84 mg (0.14 mmol, 82%) of the desired title compound after preparative HPLC (method 3).

1H NMR (300 MHz, DMSO d₆): δ (ppm)=2.15 (s, 3H), 2.20 (s, 3H), 5.31 (s, 2H), 7.30 (d, 2H), 7.38 (d, 2H), 8.15 (dd, 1H), 8.23 (d, 1H), 8.30 (s, 1H), 8.50 (d, 1H), 10.19 (s, 1H).

### Example 57

### 6,8-dichloro-N-[3,5-dimethyl-1-(2,4,6-trifluorobenzyl)-1H-pyrazol-4-yl]-2-(trifluoromethyl)quinoline-4-carboxamide

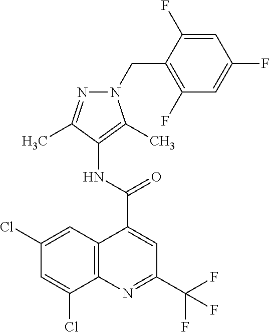 [SMILES: Cc1nn(Cc2c(F)cc(F)cc2F)c(C)c1NC(=O)c1cc(C(F)(F)F)nc2c(Cl)cc(Cl)cc12]

In analogy to example 1), 50 mg (0.20 mmol) 3,5-dimethyl-1-(2,4,6-trifluorobenzyl)-1H-pyrazol-4-amine (intermediate 7C) was stirred with 73 mg (0.24 mmol) 6,8-dichloro-2-(trifluoromethyl)quinoline-4-carboxylic acid (intermediate 11A), 51 μL (0.29 mmol) N,N-diisopropylethylamine and 94 mg (0.29 mmol) TBTU in 5 mL tetrahydrofuran for 24 h at 25° C. The reaction mixture was evaporated and dissolved in acetonitrile. The precipitated product was isolated by filtration, washed with acetonitrile and dried in high vacuum to obtain 60 mg (0.11 mmol, 56%) of the desired title compound after preparative HPLC (method 3).

1H NMR (300 MHz, DMSO d₆): δ (ppm)=2.06 (s, 3H), 2.31 (s, 3H), 5.21 (s, 2H), 7.26 (t, 2H), 8.26-8.33 (m, 1H), 8.37-8.45 (m, 2H), 10.20 (s, 1H).

### Example 58

### N-{3,5-dimethyl-1-[4-(trifluoromethoxy)benzyl]-1H-pyrazol-4-yl}-2, 6-dimethylquinoline-4-carboxamide

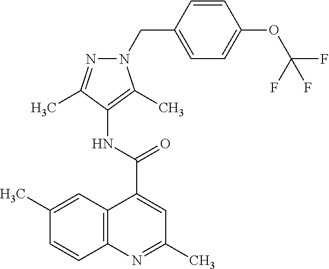 [SMILES: Cc1ccc2nc(C)cc(C(=O)Nc3c(C)nn(Cc4ccc(OC(F)(F)F)cc4)c3C)c2c1]

In analogy to example 1), 100 mg (0.18 mmol, 50%) 3,5-dimethyl-1-[4-(trifluoromethoxy)benzyl]-1H-pyrazol-4-amine (intermediate 19C) was stirred with 42 mg (0.21 mmol) 2,6-dimethylquinoline-4-carboxylic acid, 46 μL (0.26 mmol) N,N-diisopropylethylamine and 84 mg (0.26 mmol) TBTU in 3 mL tetrahydrofuran for 24 h at 25° C. to obtain 77 mg (0.16 mmol, 94%) of the desired title compound after preparative HPLC (method 3).

1H NMR (400 MHz, DMSO d₆): δ (ppm)=2.14 (s, 3H), 2.19 (s, 3H), 2.69 (s, 3H), 5.29 (s, 2H), 7.31 (d, 2H), 7.37 (d, 2H), 7.55-7.64 (m, 2H), 7.85-7.92 (m, 2H), 9.85 (s, 1H).

### Example 59

### N-(1-benzyl-3,5-dimethyl-1H-pyrazol-4-yl)-6,8-dichloro-2-(trifluoromethyl)quinoline-4-carboxamide

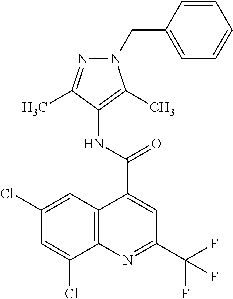 [SMILES: Cc1nn(Cc2ccccc2)c(C)c1NC(=O)c1cc(C(F)(F)F)nc2c(Cl)cc(Cl)cc12]

In analogy to example 1), 50 mg (0.25 mmol) 1-benzyl-3,5-dimethyl-1H-pyrazol-4-amine (intermediate 30C) was stirred with 92 mg (0.30 mmol) 6,8-dichloro-2-(trifluoromethyl)quinoline-4-carboxylic acid (intermediate 11A), 65 μL (0.37 mmol) N,N-diisopropylethylamine and 120 mg (0.37 mmol) TBTU in 5 mL tetrahydrofuran for 24 h at 25° C. to obtain 79 mg (0.16 mmol, 63%) of the desired title compound after preparative HPLC (method 3).

1H NMR (400 MHz, DMSO d₆): δ (ppm)=2.14 (s, 3H), 2.18 (s, 3H), 5.25 (s, 2H), 7.14-7.20 (m, 2H), 7.24-7.31 (m, 1H), 7.32-7.38 (m, 2H), 8.29 (d, 1H), 8.38 (d, 1H), 8.40 (s, 1H), 10.19 (s, 1H).

### Example 60

### 6,8-dichloro-N-{3,5-dimethyl-1-[4-(trifluoromethoxy)benzyl]-1H-pyrazol-4-yl}-2-(trifluoromethyl)quinoline-4-carboxamide

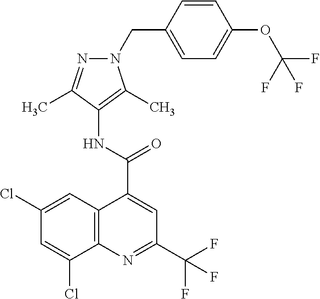 [SMILES: Cc1nn(Cc2ccc(OC(F)(F)F)cc2)c(C)c1NC(=O)c1cc(C(F)(F)F)nc2c(Cl)cc(Cl)cc12]

In analogy to example 1), 100 mg (0.18 mmol, 50%) 3,5-dimethyl-1-[4-(trifluoromethoxy)benzyl]-1H-pyrazol-4-amine (intermediate 19C) was stirred with 65 mg (0.21 mmol) 6,8-dichloro-2-(trifluoromethyl)quinoline-4-carboxylic acid (intermediate 11A), 46 μL (0.26 mmol) N,N-diisopropylethylamine and 84 mg (0.26 mmol) TBTU in 3 mL tetrahydrofuran for 24 h at 25° C. to obtain 69 mg (0.12 mmol, 68%) of the desired title compound after preparative HPLC (method 3).

1H NMR (300 MHz, DMSO d₆): δ (ppm)=2.15 (s, 3H), 2.20 (s, 3H), 5.31 (s, 2H), 7.30 (d, 2H), 7.38 (d, 2H), 8.30 (d, 1H), 8.38-8.45 (m, 2H), 10.23 (s, 1H).

### Example 61

### N-[3,5-dimethyl-1-(4-methylbenzyl)-1H-pyrazol-4-yl]-2-methoxyquinoline-4-carboxamide

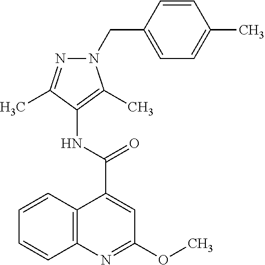 [SMILES: COc1cc(C(=O)Nc2c(C)nn(Cc3ccc(C)cc3)c2C)c2ccccc2n1]

In analogy to example 1), 50 mg (0.23 mmol) 3,5-dimethyl-1-(4-methylbenzyl)-1H-pyrazol-4-amine (intermediate 13C) was stirred with 56 mg (0.28 mmol) 2-methoxyquinoline-4-carboxylic acid, 61 μL (0.35 mmol) N,N-diisopropylethylamine and 112 mg (0.35 mmol) TBTU in 5 mL tetrahydrofuran for 24 h at 25° C. to obtain 79 mg (0.16 mmol, 63%) of the desired title compound after preparative HPLC (method 3).

1H NMR (400 MHz, DMSO d₆): δ (ppm)=2.13 (s, 3H), 2.15 (s, 3H), 2.28 (s, 3H), 4.04 (s, 3H), 5.19 (s, 2H), 7.07 (d, 2H), 7.16 (d, 2H), 7.21 (s, 1H), 7.51 (ddd, 1H), 7.73 (ddd, 1H), 7.86 (d, 1H), 8.02-8.08 (m, 1H), 9.87 (s, 1H).

### Example 62

### N-[1-(4-fluorobenzyl)-3,5-dimethyl-1H-pyrazol-4-yl]-2,6-dimethylquinoline-4-carboxamide

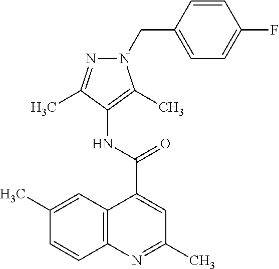 [SMILES: Cc1ccc2nc(C)cc(C(=O)Nc3c(C)nn(Cc4ccc(F)cc4)c3C)c2c1]

In analogy to example 1), 80 mg (0.36 mmol) 1-(4-fluorobenzyl)-3,5-dimethyl-1H-pyrazol-4-amine (intermediate 1C) was stirred with 88 mg (0.44 mmol) 2,6-dimethylquinoline-4-carboxylic acid, 95 μL (0.55 mmol) N,N-diisopropylethylamine and 176 mg (0.55 mmol) TBTU in 5 mL tetrahydrofuran for 3 h at 25° C. The reaction mixture was evaporated and the residue partitioned between ethyl acetate and water. The layers were separated and the aqueous layer was extracted two further times with ethyl acetate. The combined organic layers were washed with brine, dried over sodium sulfate, filtered and evaporated. The residue was dissolved in dichloromethane and under evaporation adsorbed on Isolute® HM-N (Biotage). The isolute was given on a Biotage SNAP cartridge (25 g; KP-Sil) preequilibrated with hexane and purified via column chromatography on silica gel (solvent: hexane/0-100% ethyl acetate) to obtain 119 mg (0.30 mmol, 81%) of the desired title compound

1H NMR (400 MHz, CDCl₃): δ (ppm)=2.21 (s, 3H), 2.29 (s, 3H), 2.51 (s, 3H), 2.71 (s, 3H), 5.21 (s, 2H), 7.02 (t, 2H), 7.14 (dd, 2H), 7.36 (s, 1H), 7.43 (s, 1H), 7.55 (dd, 1H), 7.92 (d, 1H), 7.98 (s, 1H).

### Example 63

### N-[1-(4-fluorobenzyl)-3,5-dimethyl-1H-pyrazol-4-yl]-6-(trifluoromethoxy)-2-(trifluoromethyl)quinoline-4-carboxamide

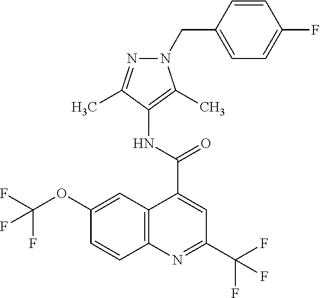 [SMILES: Cc1nn(Cc2ccc(F)cc2)c(C)c1NC(=O)c1cc(C(F)(F)F)nc2ccc(OC(F)(F)F)cc12]

In analogy to example 1), 50 mg (0.23 mmol) 1-(4-fluorobenzyl)-3,5-dimethyl-1H-pyrazol-4-amine (intermediate 1C) was stirred with 89 mg (0.27 mmol) 6-(trifluoromethoxy)-2-(trifluoromethyl)quinoline-4-carboxylic acid (intermediate 21A), 60 μL (0.34 mmol) N,N-diisopropylethylamine and 110 mg (0.34 mmol) TBTU in 3 mL tetrahydrofuran for 3 h at 25° C. to obtain 108 mg (0.21 mmol, 90%) of the desired title compound after preparative HPLC (method 3).

1H NMR (300 MHz, DMSO d₆): δ (ppm)=2.14 (s, 3H), 2.19 (s, 3H), 5.25 (s, 2H), 7.14-7.29 (m, 4H), 8.02 (dd, 1H), 8.22 (s, 1H), 8.36 (s, 1H), 8.45 (d, 1H), 10.19 (s, 1H).

### Example 64

### N-[1-(3,4-difluorobenzyl)-3,5-dimethyl-1H-pyrazol-4-yl]-2,6-dimethylquinoline-4-carboxamide

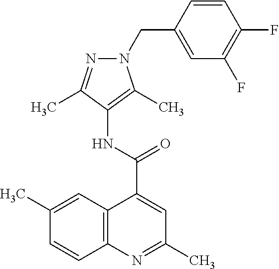 [SMILES: Cc1ccc2nc(C)cc(C(=O)Nc3c(C)nn(Cc4ccc(F)c(F)c4)c3C)c2c1]

In analogy to example 41), 50 mg (0.21 mmol) 1-(3,4-difluorobenzyl)-3,5-dimethyl-1H-pyrazol-4-amine (intermediate 4C) was stirred with 51 mg (0.25 mmol) 2,6-dimethylquinoline-4-carboxylic acid, 55 μL (0.32 mmol) N,N-diisopropylethylamine and 165 mg (0.32 mmol) PyBOP in 5 mL tetrahydrofuran for 24 h at 25° C. to obtain 35 mg (0.08 mmol, 39%) of the desired title compound after preparative HPLC (method 3).

1H NMR (400 MHz, DMSO d₆): δ (ppm)=2.15 (s, 3H), 2.19 (s, 3H), 2.70 (s, 3H), 5.25 (s, 2H), 7.03 (ddd, 1H), 7.23 (ddd, 1H), 7.44 (dt, 1H), 7.57-7.66 (m, 2H), 7.86-7.94 (m, 2H), 9.87 (s, 1H).

### Example 65

### N-(1-benzyl-3,5-dimethyl-1H-pyrazol-4-yl)-2-methoxyquinoline-4-carboxamide

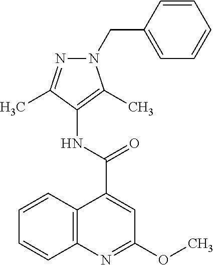 [SMILES: COc1cc(C(=O)Nc2c(C)nn(Cc3ccccc3)c2C)c2ccccc2n1]

In analogy to example 1), 50 mg (0.25 mmol) 1-benzyl-3,5-dimethyl-1H-pyrazol-4-amine (intermediate 30C) was stirred with 61 mg (0.30 mmol) 2-methoxyquinoline-4-carboxylic acid, 65 μL (0.37 mmol) N,N-diisopropytethylamine and 120 mg (0.37 mmol) TBTU in 5 mL tetrahydrofuran for 24 h at 25° C. to obtain 45 mg (0.12 mmol, 46%) of the desired title compound after preparative HPLC (method 3).

1H NMR (300 MHz, DMSO d₆): δ (ppm)=2.13 (s, 3H), 2.16 (s, 3H), 4.04 (s, 3H), 5.25 (s, 2H), 7.14-7.20 (m, 2H), 7.22 (s, 1H), 7.25-7.32 (m, 1H), 7.32-7.40 (m, 2H), 7.47-7.55 (m, 1H), 7.69-7.77 (m, 1H), 7.86 (d, 1H), 8.05 (d, 1H), 9.90 (s, 1H).

### Example 66

### 6,8-dichloro-N-[3,5-dimethyl-1-(pyridin-2-ylmethyl)-1H-pyrazol-4-yl]-2-(trifluoromethyl)quinoline-4-carboxamide

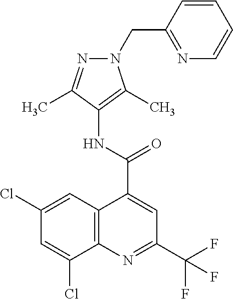 [SMILES: Cc1nn(Cc2ccccn2)c(C)c1NC(=O)c1cc(C(F)(F)F)nc2c(Cl)cc(Cl)cc12]

In analogy to example 1), 50 mg (0.25 mmol) 3,5-dimethyl-1-(pyridin-2-ylmethyl)-1H-pyrazol-4-amine (intermediate 18C) was stirred with 92 mg (0.30 mmol) 6,8-dichloro-2-(trifluoromethyl)quinoline-4-carboxylic acid (intermediate 11A), 65 μL (0.37 mmol) N,N-diisopropylethylamine and 120 mg (0.37 mmol) TBTU in 5 mL tetrahydrofuran for 24 h at 25° C. to obtain 35 mg (0.07 mmol, 28%) of the desired title compound after preparative HPLC (method 3).

1H NMR (300 MHz, DMSO d₆): δ (ppm)=2.14 (s, 3H), 2.24 (s, 3H), 5.34 (s, 2H), 7.04 (d, 1H), 7.28-7.35 (m, 1H), 7.80 (td, 1H), 8.31 (d, 1H), 8.40 (d, 1H), 8.42 (s, 1H), 8.51-8.57 (m, 1H), 10.22 (s, 1H).

### Example 67

### 6,8-dichloro-N-[1-(3-methoxybenzyl)-3,5-dimethyl-1H-pyrazol-4-yl]-2-(trifluoromethyl)quinoline-4-carboxamide

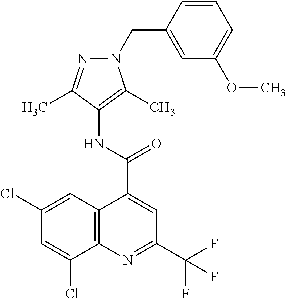 [SMILES: COc1cccc(Cn2nc(C)c(NC(=O)c3cc(C(F)(F)F)nc4c(Cl)cc(Cl)cc34)c2C)c1]

In analogy to example 1), 50 mg (0.22 mmol) 1-(3-methoxybenzyl)-3,5-dimethyl-1H-pyrazol-4-amine (intermediate 12C) was stirred with 80 mg (0.26 mmol) 6,8-dichloro-2-(trifluoromethyl)quinoline-4-carboxylic acid (intermediate 11A), 56 μL (0.32 mmol) N,N-diisopropylethylamine and 104 mg (0.32 mmol) TBTU in 5 mL tetrahydrofuran for 24 h at 25° C. to obtain 71 mg (0.13 mmol, 62%) of the desired title compound after preparative HPLC (method 3).

1H NMR (300 MHz, DMSO d₆): δ (ppm)=2.15 (s, 3H), 2.18 (s, 3H), 3.73 (s, 3H), 5.23 (s, 2H), 6.68-6.74 (m, 2H), 6.82-6.89 (m, 1H), 7.27 (t, 1H), 8.30 (d, 1H), 8.41 (d, 1H), 8.43 (s, 1H), 10.22 (s, 1H).

### Example 68

### 6,8-dichloro-N-[1-(2-cyanobenzyl)-3,5-dimethyl-1H-pyrazol-4-yl]-2-(trifluoromethyl)quinoline-4-carboxamide

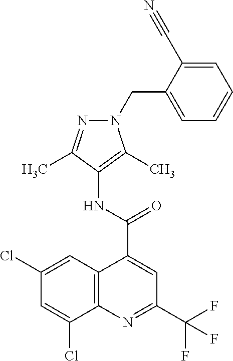 [SMILES: Cc1nn(Cc2ccccc2C#N)c(C)c1NC(=O)c1cc(C(F)(F)F)nc2c(Cl)cc(Cl)cc12]

In analogy to example 1), 50 mg (0.22 mmol) 2-[(4-amino-3,5-dimethyl-1H-pyrazol-1-yl)methyl]benzonitrile (intermediate 10C) was stirred with 82 mg (0.27 mmol) 6,8-dichloro-2-(trifluoromethyl)quinoline-4-carboxylic acid (intermediate 11A), 58 μL (0.33 mmol) N,N-diisopropylethylamine and 106 mg (0.33 mmol) TBTU in 5 mL tetrahydrofuran for 24 h at 25° C. to obtain 68 mg (0.13 mmol, 58%) of the desired title compound after preparative HPLC (method 3).

1H NMR (400 MHz, DMSO d₆): δ (ppm)=2.14 (s, 3H), 2.27 (s, 3H), 5.44 (s, 2H), 7.11 (d, 1H), 7.50-7.56 (m, 1H), 7.71 (td, 1H), 7.90 (dd, 1H), 8.31 (d, 1H), 8.40 (d, 1H), 8.43 (s, 1H), 10.25 (s, 1H).

### Example 69

### 2-ethyl-N-[1-(4-fluorobenzyl)-3,5-dimethyl-1H-pyrazol-4-yl]quinoline-4-carboxamide

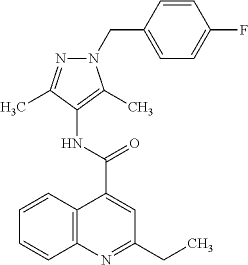 [SMILES: CCc1cc(C(=O)Nc2c(C)nn(Cc3ccc(F)cc3)c2C)c2ccccc2n1]

100 mg (0.22 mmol) 2-Bromo-N-[1-(4-fluorobenzyl)-3,5-dimethyl-1H-pyrazol-4-yl]quinoline-4-carboxamide (example 36) were dissolved in 5 mL dry tetrahydrofuran and cooled to 0° C. To this solution was added 110 μL (0.33 mmol, 3 M in tetrahydrofuran) ethyl magnesium bromide solution and the reaction mixture was stirred for 4 h at 25° C. The reaction mixture was evaporated and the residue partitioned between water and ethyl acetate. After two further extractions with ethyl acetate, the combined organic layers were washed with brine, dried over sodium sulfate, filtered and evaporated. The crude mixture was pre-purified via preparative HPLC (method 3) and another preparative HPLC (method 5c) was done to obtain 68 mg (0.13 mmol, 58%) of the desired title compound.

1H NMR (300 MHz, CDCl₃): δ (ppm)=1.41 (t, 3H), 2.27 (s, 3H), 2.34 (s, 3H), 3.18 (q, 2H), 5.36 (s, 2H), 7.00-7.11 (m, 2H), 7.11-7.21 (m, 2H), 7.82-7.92 (m, 2H), 7.98 (t, 1H), 8.26 (d, 1H), 8.41 (d, 1H), 9.39 (s, 1H).

### Example 70

### 6-bromo-N-[3,5-dimethyl-1-(pyridin-2-ylmethyl)-1H-pyrazol-4-yl]-2-(trifluoromethyl)quinoline-4-carboxamide

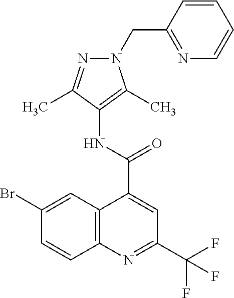 [SMILES: Cc1nn(Cc2ccccn2)c(C)c1NC(=O)c1cc(C(F)(F)F)nc2ccc(Br)cc12]

In analogy to example 1), 50 mg (0.25 mmol) 3,5-dimethyl-1-(pyridin-2-ylmethyl)-1H-pyrazol-4-amine (intermediate 18C) was stirred with 95 mg (0.30 mmol) 6-bromo-2-(trifluoromethyl)quinoline-4-carboxylic acid (intermediate 1A), 65 μL (0.37 mmol) N,N-diisopropylethylamine and 119 mg (0.37 mmol) TBTU in 5 mL tetrahydrofuran for 24 h at 25° C. to obtain 52 mg (0.10 mmol, 41%) of the desired title compound after preparative HPLC (method 3).

1H NMR (300 MHz, DMSO d₆): δ (ppm)=2.14 (s, 3H), 2.24 (s, 3H), 5.34 (s, 2H), 7.05 (d, 1H), 7.28-7.36 (m, 1H), 7.80 (td, 1H), 8.15 (dd, 1H), 8.24 (d, 1H), 8.30 (s, 1H), 8.51 (d, 1H), 8.53-8.57 (m, 1H), 10.18 (s, 1H).

### Example 71

### 6-bromo-N-[1-(3-cyanobenzyl)-3,5-dimethyl-1H-pyrazol-4-yl]-2-(trifluoromethyl)quinoline-4-carboxamide

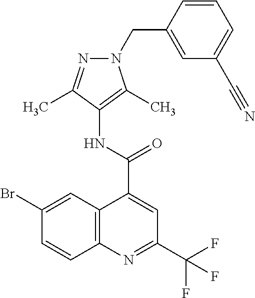 [SMILES: Cc1nn(Cc2cccc(C#N)c2)c(C)c1NC(=O)c1cc(C(F)(F)F)nc2ccc(Br)cc12]

In analogy to example 1), 50 mg (0.22 mmol) 3-[(4-amino-3,5-dimethyl-1H-pyrazol-1-yl)methyl]benzonitrile (intermediate 9C) was stirred with 85 mg (0.27 mmol) 6-bromo-2-(trifluoromethyl)quinoline-4-carboxylic acid (intermediate 1A), 58 μL (0.33 mmol) N,N-diisopropylethylamine and 106 mg (0.33 mmol) TBTU in 5 mL tetrahydrofuran for 24 h at 25° C. to obtain 86 mg (0.16 mmol, 73%) of the desired title compound after preparative HPLC (method 3).

1H NMR (400 MHz, DMSO d₆): δ (ppm)=2.16 (s, 3H), 2.21 (s, 3H), 5.34 (s, 2H), 7.50 (d, 1H), 7.57-7.64 (m, 2H), 7.79 (d, 1H), 8.15 (dd, 1H), 8.23 (d, 1H), 8.30 (s, 1H), 8.51 (d, 1H), 10.19 (s, 1H).

### Example 72

### 6-bromo-N-[1-(3-fluorobenzyl)-3,5-dimethyl-1H-pyrazol-4-yl]-2-(trifluoromethyl)quinoline-4-carboxamide

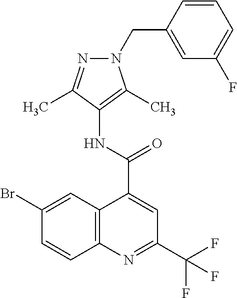 [SMILES: Cc1nn(Cc2cccc(F)c2)c(C)c1NC(=O)c1cc(C(F)(F)F)nc2ccc(Br)cc12]

In analogy to example 1), 50 mg (0.23 mmol) 1-(3-fluorobenzyl)-3,5-dimethyl-1H-pyrazol-4-amine (intermediate 2C) was stirred with 88 mg (0.27 mmol) 6-bromo-2-(trifluoromethyl)quinoline-4-carboxylic acid (intermediate 1A), 60 μL (0.34 mmol) N,N-diisopropylethylamine and 110 mg (0.34 mmol) TBTU in 5 mL tetrahydrofuran for 24 h at 25° C. to obtain 72 mg (0.13 mmol, 59%) of the desired title compound after preparative HPLC (method 3).

1H NMR (300 MHz, DMSO d₆): δ (ppm)=2.17 (s, 3H), 2.20 (s, 3H), 5.30 (s, 2H), 6.89-7.07 (m, 2H), 7.07-7.22 (m, 1H), 7.34-7.49 (m, 1H), 8.15 (dd, 1H), 8.24 (d, 1H), 8.30 (s, 1H), 8.52 (d, 1H), 10.18 (s, 1H).

### Example 73

### N-[1-(4-fluorobenzyl)-5-methyl-3-(trifluoromethyl)-1H-pyrazol-4-yl]-2,6-dimethylquinoline-4-carboxamide

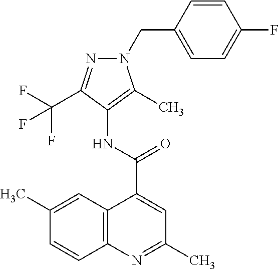 [SMILES: Cc1ccc2nc(C)cc(C(=O)Nc3c(C(F)(F)F)nn(Cc4ccc(F)cc4)c3C)c2c1]

In analogy to example 1), 75 mg (0.20 mmol, 75%) 1-(4-fluorobenzyl)-5-methyl-3-(trifluoromethyl)-1H-pyrazol-4-amine (intermediate 25C) was stirred with 49 mg (0.24 mmol) 2,6-dimethylquinoline-4-carboxylic acid, 53 μL (0.30 mmol) N,N-diisopropylethylamine and 98 mg (0.30 mmol) TBTU in 3 mL tetrahydrofuran for 24 h at 25° C. to obtain 55 mg (0.12 mmol, 58%) of the desired title compound after preparative HPLC (method 3).

1H NMR (300 MHz, DMSO d₆): δ (ppm)=2.27 (s, 3H), 2.70 (s, 3H), 5.46 (s, 2H), 7.20-7.36 (m, 4H), 7.52 (s, 1H), 7.62 (dd, 1H), 7.82 (s, 1H), 7.90 (d, 1H), 10.20 (s, 1H).

### Example 74

### 6-bromo-N-[1-(2-fluorobenzyl)-3,5-dimethyl-1H-pyrazol-4-yl]-2-(trifluoromethyl)quinoline-4-carboxamide

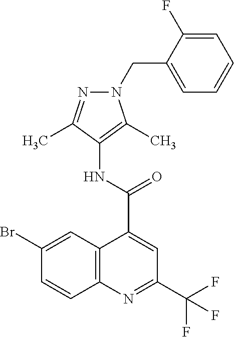 [SMILES: Cc1nn(Cc2ccccc2F)c(C)c1NC(=O)c1cc(C(F)(F)F)nc2ccc(Br)cc12]

In analogy to example 1), 50 mg (0.23 mmol) 1-(2-fluorobenzyl)-3,5-dimethyl-1H-pyrazol-4-amine (intermediate 3C) was stirred with 88 mg (0.27 mmol) 6-bromo-2-(trifluoromethyl)quinoline-4-carboxylic acid (intermediate 1A), 60 μL (0.34 mmol) N,N-diisopropylethylamine and 110 mg (0.34 mmol) TBTU in 5 mL tetrahydrofuran for 24 h at 25° C. to obtain 77 mg (0.14 mmol, 60%) of the desired title compound after preparative HPLC (method 3).

1H NMR (400 MHz, DMSO d₆): δ (ppm)=2.14 (s, 3H), 2.23 (s, 3H), 5.30 (s, 2H), 7.03-7.10 (m, 1H), 7.16-7.28 (m, 2H), 7.34-7.41 (m, 1H), 8.15 (dd, 1H), 8.23 (d, 1H), 8.30 (s, 1H), 8.51 (d, 1H), 10.17 (s, 1H).

### Example 75

### N-[1-(4-fluorobenzyl)-3,5-dimethyl-1H-pyrazol-4-yl]-2-hydroxy-6-methoxyquinoline-4-carboxamide

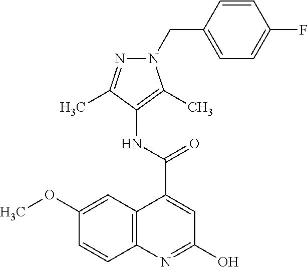 [SMILES: COc1ccc2nc(O)cc(C(=O)Nc3c(C)nn(Cc4ccc(F)cc4)c3C)c2c1]

In analogy to example 1), 100 mg (0.46 mmol) 1-(4-fluorobenzyl)-3,5-dimethyl-1H-pyrazol-4-amine (intermediate 1C) was stirred with 120 mg (0.55 mmol) 2-hydroxy-6-methoxyquinoline-4-carboxylic acid, 119 μL (0.68 mmol) N,N-diisopropylethylamine and 220 mg (0.68 mmol) TBTU in 5 mL tetrahydrofuran for 3 h at 25° C. The reaction mixture was evaporated and the residue partitioned between ethyl acetate and water. The layers were separated and the aqueous layer was extracted two further times with ethyl acetate. The combined organic layers were washed with brine, dried over sodium sulfate, filtered and evaporated. The residue was dissolved in dichloromethane and under evaporation adsorbed on Isolute® HM-N (Biotage). The isolute was given on a Biotage SNAP cartridge (25 g; KP-Sil) preequilibrated with hexane and purified via column chromatography on silica gel (solvent: dichloromethane/0-10% methanol). The resulting crude product was dissolved in N,N-dimethylformamide and the resulting precipitate was isolated by filtration, washed with tert-butyl methyl ether and dried under high vacuum to obtain 119 mg (0.28 mmol, 62%) of the desired title compound.

1H NMR (400 MHz, DMSO d₆): δ (ppm)=2.12 (s, 3H), 2.16 (s, 3H), 3.75 (s, 3H), 5.23 (s, 2H), 6.71 (s, 1H), 7.15-7.28 (m, 6H), 7.34 (d, 1H), 9.88 (s, 1H), 11.89 (s, 1H).

### Example 76

### N-[3,5-dimethyl-1-(3-methylbenzyl)-1H-pyrazol-4-yl]-2-methoxyquinoline-4-carboxamide

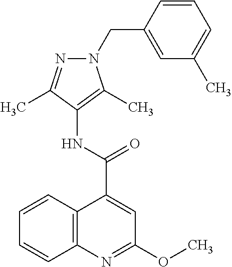 [SMILES: COc1cc(C(=O)Nc2c(C)nn(Cc3cccc(C)c3)c2C)c2ccccc2n1]

In analogy to example 1), 50 mg (0.23 mmol) 3,5-dimethyl-1-(3-methylbenzyl)-1H-pyrazol-4-amine (intermediate 14C) was stirred with 57 mg (0.28 mmol) 2-methoxyquinoline-4-carboxylic acid, 61 μL (0.35 mmol) N,N-diisopropylethylamine and 112 mg (0.35 mmol) TBTU in 5 mL tetrahydrofuran for 24 h at 25° C. to obtain 68 mg (0.17 mmol, 72%) of the desired title compound after preparative HPLC (method 3).

1H NMR (300 MHz, DMSO d₆): δ (ppm)=2.12 (s, 3H), 2.15 (s, 3H), 2.28 (s, 3H), 4.03 (s, 3H), 5.19 (s, 2H), 6.94 (d, 1H), 7.02 (s, 1H), 7.09 (d, 1H), 7.19-7.27 (m, 2H), 7.50 (td, 1H), 7.72 (td, 1H), 7.85 (d, 1H), 8.04 (d, 1H), 9.89 (s, 1H).

### Example 77

### N-[1-(2-fluorobenzyl)-3,5-dimethyl-1H-pyrazol-4-yl]-2-methoxyquinoline-4-carboxamide

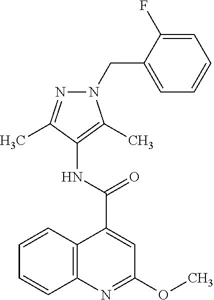 [SMILES: COc1cc(C(=O)Nc2c(C)nn(Cc3ccccc3F)c2C)c2ccccc2n1]

In analogy to example 41), 100 mg (0.46 mmol) 1-(2-fluorobenzyl)-3,5-dimethyl-1H-pyrazol-4-amine (intermediate 3C) was stirred with 111 mg (0.55 mmol) 2-methoxyquinoline-4-carboxylic acid, 119 μL (0.68 mmol) N,N-diisopropylethylamine and 356 mg (0.68 mmol) PyBOP in 5 mL tetrahydrofuran for 24 h at 25° C. to obtain 115 mg (0.28 mmol, 62%) of the desired title compound after preparative HPLC (method 3).

1H NMR (300 MHz, DMSO d₆): δ (ppm)=2.11 (s, 3H), 2.21 (s, 3H), 4.04 (s, 3H), 5.29 (s, 2H), 6.99-7.10 (m, 1H), 7.15-7.29 (m, 3H), 7.32-7.42 (m, 1H), 7.47-7.57 (m, 1H), 7.69-7.79 (m, 1H), 7.86 (d, 1H), 8.05 (d, 1H), 9.92 (s, 1H).

### Example 78

### 2-cyano-N-[1-(4-fluorobenzyl)-3,5-dimethyl-1H-pyrazol-4-yl]quinoline-4-carboxamide

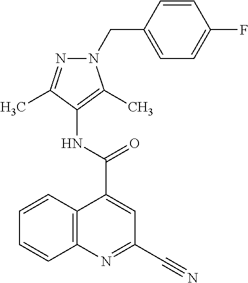 [SMILES: Cc1nn(Cc2ccc(F)cc2)c(C)c1NC(=O)c1cc(C#N)nc2ccccc12]

To a solution of 100 mg (0.17 mmol) 2-bromo-N-[1-(4-fluorobenzyl)-3,5-dimethyl-1H-pyrazol-4-yl]quinoline-4-carboxamide (example 36) in degassed 2.0 mL N,N-dimethylformamide was added 28 mg (0.24 mmol) zinc cyanide and 25 mg (0.02 mmol) tetrakis(triphenylphospine)palladium (0) and the reaction mixture was heated for 10 minutes at 150° C. in a microwave. The reaction suspension was poured on a biphasic mixture of water and ethyl acetate and the aqueous layer was extracted with ethyl acetate two further times. The combined organic layers were washed with brine, dried over sodium sulfate, filtered and evaporated. The residue was dissolved in 2.5 mL N,N-dimethylformamide and purified via preparative HPLC (method 3) to obtain 54 mg (0.14 mmol, 61%) of the desired title compound after drying.

1H NMR (300 MHz, DMSO d₆): δ (ppm)=2.15 (s, 3H), 2.19 (s, 3H), 5.24 (s, 2H), 7.15-7.27 (m, 4H), 7.88-7.95 (m, 1H), 7.97-8.05 (m, 1H), 8.20-8.29 (m, 2H), 8.36 (s, 1H), 10.07 (s, 1H).

### Example 79

### 2-cyclopropyl-N-[1-(4-fluorobenzyl)-3,5-dimethyl-1H-pyrazol-4-yl]-6-(trifluoromethyl)quinoline-4-carboxamide

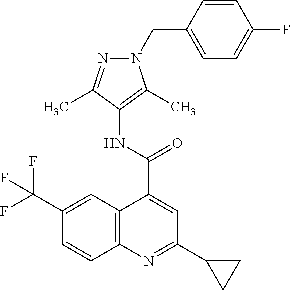 [SMILES: Cc1nn(Cc2ccc(F)cc2)c(C)c1NC(=O)c1cc(C2CC2)nc2ccc(C(F)(F)F)cc12]

In analogy to example 1), 13 mg (0.06 mmol) 1-(4-fluorobenzyl)-3,5-dimethyl-1H-pyrazol-4-amine (intermediate 1C) was stirred with 20 mg (0.07 mmol) 2-cyclopropyl-6-(trifluoromethyl)quinoline-4-carboxylic acid (intermediate 34A), 15 μL (0.09 mmol) N,N-diisopropylethylamine and 29 mg (0.09 mmol) TBTU in 5 mL tetrahydrofuran for 24 h at 25° C. to obtain 25 mg (0.05 mmol, 85%) of the desired title compound after preparative HPLC (method 3).

1H NMR (400 MHz, DMSO d₆): δ (ppm)=1.14-1.24 (m, 4H), 2.14 (s, 3H), 2.18 (s, 3H), 2.42-2.48 (m, 1H), 5.25 (s, 2H), 7.14-7.31 (m, 4H), 7.84 (s, 1H), 8.00 (dd, 1H), 8.12 (d, 1H), 8.50 (s, 1H), 10.02 (s, 1H).

### Example 80

### N-[1-(2-cyanobenzyl)-3,5-dimethyl-1H-pyrazol-4-yl]-2-methoxyquinoline-4-carboxamide

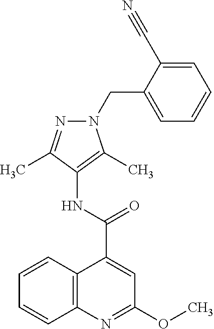 [SMILES: COc1cc(C(=O)Nc2c(C)nn(Cc3ccccc3C#N)c2C)c2ccccc2n1]

In analogy to example 1), 50 mg (0.22 mmol) 2-[(4-amino-3,5-dimethyl-1H-pyrazol-1-yl)methyl]benzonitrile (intermediate 10C) was stirred with 54 mg (0.27 mmol) 2-methoxyquinoline-4-carboxylic acid, 58 μL (0.33 mmol) N,N-diisopropylethylamine and 106 mg (0.33 mmol) TBTU in 5 mL tetrahydrofuran for 24 h at 25° C. to obtain 15 mg (0.03 mmol, 16%) of the desired title compound after preparative HPLC (method 3).

1H NMR (400 MHz, DMSO d₆): δ (ppm)=2.12 (s, 3H), 2.24 (s, 3H), 4.05 (s, 3H), 5.43 (s, 2H), 7.09 (d, 1H), 7.24 (s, 1H), 7.49-7.56 (m, 2H), 7.68-7.77 (m, 2H), 7.84-7.93 (m, 2H), 8.06 (d, 1H), 9.94 (s, 1H).

### Example 81

### 2-methoxy-N-[1-(3-methoxybenzyl)-3,5-dimethyl-1H-pyrazol-4-yl]quinoline-4-carboxamide

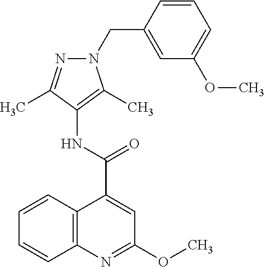 [SMILES: COc1cccc(Cn2nc(C)c(NC(=O)c3cc(OC)nc4ccccc34)c2C)c1]

In analogy to example 1), 50 mg (0.22 mmol) 1-(3-methoxybenzyl)-3,5-dimethyl-1H-pyrazol-4-amine (intermediate 12C) was stirred with 53 mg (0.26 mmol) 2-methoxyquinoline-4-carboxylic acid, 60 μL (0.35 mmol) N,N-diisopropylethylamine and 111 mg (0.35 mmol) TBTU in 5 mL tetrahydrofuran for 24 h at 25° C. to obtain 35 mg (0.07 mmol, 29%) of the desired title compound after preparative HPLC (method 3).

1H NMR (400 MHz, DMSO d₆): δ (ppm)=2.14 (s, 3H), 2.16 (s, 3H), 3.73 (s, 3H), 4.04 (s, 3H), 5.22 (s, 2H), 6.69-6.74 (m, 2H), 6.83-6.88 (m, 1H), 7.22 (s, 1H), 7.27 (t, 1H), 7.51 (ddd, 1H), 7.73 (ddd, 1H), 7.83-7.89 (m, 1H), 8.06 (dd, 1H), 9.88 (s, 1H).

### Example 82

### 6,8-dichloro-N-{3,5-dimethyl-1-[(3-methylpyridin-2-yl)methyl]-1H-pyrazol-4-yl}-2-(trifluoromethyl)quinoline-4-carboxamide

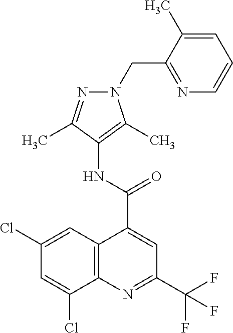 [SMILES: Cc1cccnc1Cn1nc(C)c(NC(=O)c2cc(C(F)(F)F)nc3c(Cl)cc(Cl)cc23)c1C]

In analogy to example 1), 50 mg (0.23 mmol) 3,5-dimethyl-1-[(3-methylpyridin-2-yl)methyl]-1H-pyrazol-4-amine (intermediate 36C) was stirred with 86 mg (0.28 mmol) 6,8-dichloro-2-(trifluoromethyl)quinoline-4-carboxylic acid (intermediate 11A), 60 μL (0.35 mmol) N,N-diisopropylethylamine and 111 mg (0.35 mmol) TBTU in 5 mL tetrahydrofuran for 24 h at 25° C. to obtain 35 mg (0.07 mmol, 29%) of the desired title compound after preparative HPLC (method 3).

1H NMR (400 MHz, DMSO d₆): δ (ppm)=2.14 (s, 3H), 2.24 (s, 3H), 2.47 (s, 3H), 5.28 (s, 2H), 6.72 (d, 1H), 7.17 (d, 1H), 7.66 (t, 1H), 8.31 (d, 1H), 8.40 (d, 1H), 8.43 (s, 1H), 10.23 (s, 1H).

### Example 83

### 6-bromo-N-{3,5-dimethyl-1-[2-(trifluoromethoxy)benzyl]-1H-pyrazol-4-yl}-2-(trifluoromethyl)quinoline-4-carboxamide

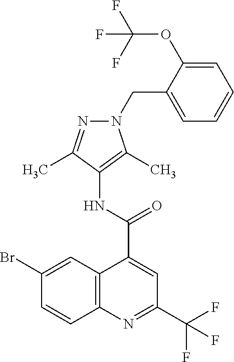 [SMILES: Cc1nn(Cc2ccccc2OC(F)(F)F)c(C)c1NC(=O)c1cc(C(F)(F)F)nc2ccc(Br)cc12]

In analogy to example 1), 100 mg (0.18 mmol, 50%) 3,5-dimethyl-1-[2-(trifluoromethoxy)benzyl]-1H-pyrazol-4-amine (intermediate 21C) was stirred with 67 mg (0.21 mmol) 6-bromo-2-(trifluoromethyl)quinoline-4-carboxylic acid (intermediate 1A), 45 μL (0.26 mmol) N,N-diisopropylethylamine and 84 mg (0.26 mmol) TBTU in 3 mL tetrahydrofuran for 24 h at 25° C. to obtain 68 mg (0.11 mmol, 60%) of the desired title compound after preparative HPLC (method 3).

1H NMR (300 MHz, DMSO d₆): δ (ppm)=2.15 (s, 3H), 2.18-2.22 (m, 3H), 5.32 (s, 2H), 6.96 (d, 1H), 7.33-7.50 (m, 3H), 8.15 (dd, 1H), 8.23 (d, 1H), 8.32 (s, 1H), 8.51 (d, 1H), 10.22 (s, 1H).

### Example 84

### N-[1-(2,6-difluorobenzyl)-3,5-dimethyl-1H-pyrazol-4-yl]-2,6-dimethylquinoline-4-carboxamide

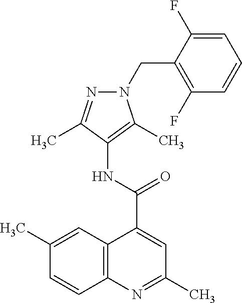 [SMILES: Cc1ccc2nc(C)cc(C(=O)Nc3c(C)nn(Cc4c(F)cccc4F)c3C)c2c1]

In analogy to example 1), 50 mg (0.21 mmol) 1-(2,6-difluorobenzyl)-3,5-dimethyl-1H-pyrazol-4-amine (intermediate 6C) was stirred with 51 mg (0.25 mmol) 2,6-dimethylquinoline-4-carboxylic acid, 55 μL (0.32 mmol) N,N-diisopropylethylamine and 101 mg (0.32 mmol) TBTU in 3 mL tetrahydrofuran for 24 h at 25° C. to obtain 59 mg (0.14 mmol, 66%) of the desired title compound after preparative HPLC (method 3).

1H NMR (300 MHz, DMSO d₆): δ (ppm)=2.06 (s, 3H), 2.30 (s, 3H), 2.70 (s, 3H), 5.24 (s, 2H), 7.15 (t, 2H), 7.41-7.54 (m, 1H), 7.55-7.65 (m, 2H), 7.84-7.93 (m, 2H), 9.84 (s, 1H).

### Example 85

### 8-fluoro-N-[1-(4-fluorobenzyl)-3,5-dimethyl-1H-pyrazol-4-yl]-2-(trifluoromethyl)quinoline-4-carboxamide

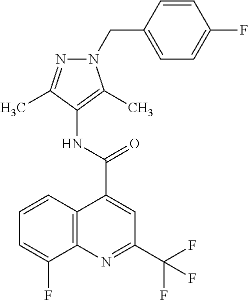 [SMILES: Cc1nn(Cc2ccc(F)cc2)c(C)c1NC(=O)c1cc(C(F)(F)F)nc2c(F)cccc12]

In analogy to example 1), 43 mg (0.20 mmol) 1-(4-fluorobenzyl)-3,5-dimethyl-1H-pyrazol-4-amine (intermediate 1C) was stirred with 61 mg (0.24 mmol) 8-fluoro-2-(trifluoromethyl)quinoline-4-carboxylic acid (intermediate 35A), 51 μL (0.29 mmol) N,N-diisopropylethylamine and 94 mg (0.29 mmol) TBTU in 3 mL tetrahydrofuran for 2 h at 25° C. to obtain 65 mg (0.14 mmol, 66%) of the desired title compound after preparative HPLC (method 3).

1H NMR (400 MHz, DMSO d₆): δ (ppm)=2.15 (s, 3H), 2.19 (s, 3H), 5.25 (s, 2H), 7.15-7.26 (m, 4H), 7.83-7.94 (m, 2H), 8.07-8.12 (m, 1H), 8.30 (s, 1H), 10.12 (s, 1H).

### Example 86

### N-[1-(4-fluorobenzyl)-3,5-dimethyl-1H-pyrazol-4-yl]-2,8-dimethylquinoline-4-carboxamide

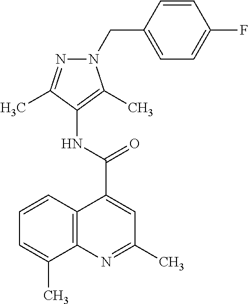 [SMILES: Cc1cc(C(=O)Nc2c(C)nn(Cc3ccc(F)cc3)c2C)c2cccc(C)c2n1]

In analogy to example 1), 100 mg (0.46 mmol) 1-(4-fluorobenzyl)-3,5-dimethyl-1H-pyrazol-4-amine (intermediate 1C) was stirred with 110 mg (0.55 mmol) 2,8-dimethylquinoline-4-carboxylic acid, 119 μL (0.68 mmol) N,N-diisopropylethylamine and 219 mg (0.68 mmol) TBTU in 5 mL tetrahydrofuran for 3 h at 25° C. The reaction mixture was evaporated and the residue partitioned between ethyl acetate and water. The layers were separated and the aqueous layer was extracted two further times with ethyl acetate. The combined organic layers were washed with brine, dried over sodium sulfate, filtered and evaporated. The residue was dissolved in dichloromethane and under evaporation adsorbed on Isolute® HM-N (Biotage). The isolute was given on a Biotage SNAP cartridge (25 g; KP-Sil) preequilibrated with hexane and purified via column chromatography on silica gel (solvent: hexane/0-100% ethyl acetate). The resulting crude product was again purified via preparative HPLC (method 3) to obtain 136 mg (0.33 mmol, 73%) of the desired title compound.

1H NMR (400 MHz, CDCl₃): δ (ppm)=2.22 (s, 3H), 2.30 (s, 3H), 2.80 (s, 3H), 2.82 (s, 3H), 5.23 (s, 2H), 6.98-7.08 (m, 3H), 7.11-7.18 (m, 2H), 7.42-7.51 (m, 2H), 7.60 (d, 1H), 8.07 (d, 1H).

### Example 87

### N-[1-(3-fluorobenzyl)-3,5-dimethyl-1H-pyrazol-4-yl]-2-methoxyquinoline-4-carboxamide

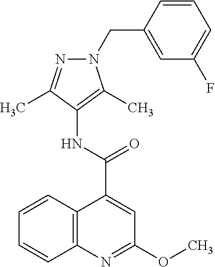 [SMILES: COc1cc(C(=O)Nc2c(C)nn(Cc3cccc(F)c3)c2C)c2ccccc2n1]

In analogy to example 1), 50 mg (0.23 mmol) 1-(4-fluorobenzyl)-3,5-dimethyl-1H-pyrazol-4-amine (intermediate 2C) was stirred with 56 mg (0.27 mmol) 2-methoxyquinoline-4-carboxylic acid, 60 μL (0.34 mmol) N,N-diisopropylethylamine and 110 mg (0.34 mmol) TBTU in 5 mL tetrahydrofuran for 24 h at 25° C. to obtain 56 mg (0.13 mmol, 59%) of the desired title compound after preparative HPLC (method 3).

1H NMR (400 MHz, DMSO d₆): δ (ppm)=2.14 (s, 3H), 2.17 (s, 3H), 4.04 (s, 3H), 5.28 (s, 2H), 6.91-6.98 (m, 1H), 7.00 (d, 1H), 7.08-7.16 (m, 1H), 7.22 (s, 1H), 7.37-7.45 (m, 1H), 7.48-7.55 (m, 1H), 7.70-7.77 (m, 1H), 7.86 (d, 1H), 8.03-8.08 (m, 1H), 9.90 (s, 1H).

### Example 88

### 7-bromo-N-[1-(4-fluorobenzyl)-3,5-dimethyl-1H-pyrazol-4-yl]-2-(trifluoromethyl)quinoline-4-carboxamide

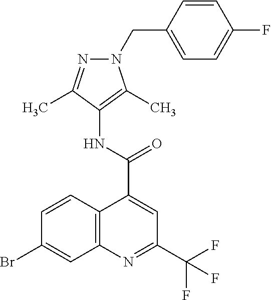 [SMILES: Cc1nn(Cc2ccc(F)cc2)c(C)c1NC(=O)c1cc(C(F)(F)F)nc2cc(Br)ccc12]

In analogy to example 1), 50 mg (0.23 mmol) 1-(4-fluorobenzyl)-3,5-dimethyl-1H-pyrazol-4-amine (intermediate 1C) was stirred with 88 mg (0.27 mmol) 7-bromo-2-(trifluoromethyl)quinoline-4-carboxylic acid (intermediate 22A), 60 μL (0.34 mmol) N,N-diisopropylethylamine and 110 mg (0.34 mmol) TBTU in 3 mL tetrahydrofuran for 24 h at 25° C. to obtain 83 mg (0.16 mmol, 70%) of the desired title compound after preparative HPLC (method 3).

1H NMR (300 MHz, DMSO d₆): δ (ppm)=2.14 (s, 3H), 2.19 (s, 3H), 5.25 (s, 2H), 7.13-7.28 (m, 4H), 8.06 (dd, 1H), 8.24 (d, 1H), 8.27 (s, 1H), 8.55 (d, 1H), 10.14 (s, 1H).

### Example 89

### 6,8-dichloro-N-[3,5-dimethyl-1-(2-methylbenzyl)-1H-pyrazol-4-yl]-2-(trifluoromethyl)quinoline-4-carboxamide

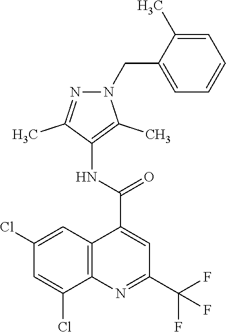 [SMILES: Cc1ccccc1Cn1nc(C)c(NC(=O)c2cc(C(F)(F)F)nc3c(Cl)cc(Cl)cc23)c1C]

In analogy to example 1), 50 mg (0.23 mmol) 3,5-dimethyl-1-(2-methylbenzyl)-1H-pyrazol-4-amine (intermediate 15C) was stirred with 86 mg (0.28 mmol) 6,8-dichloro-2-(trifluoromethyl)quinoline-4-carboxylic acid (intermediate 11A), 61 μL (0.35 mmol) N,N-diisopropylethylamine and 111 mg (0.35 mmol) TBTU in 5 mL tetrahydrofuran for 24 h at 25° C. The reaction mixture was evaporated and dissolved in water. The precipitate was isolated by filtration, washed with tetrahydrofuran and dried in high vacuum to obtain 13 mg (0.03 mmol, 11%) of the desired title compound.

1H NMR (300 MHz, DMSO d₆): δ (ppm)=2.16 (s, 6H), 2.35 (s, 3H), 5.26 (s, 2H), 6.59 (d, 1H), 7.09-7.25 (m, 3H), 8.32 (d, 1H), 8.38-8.50 (m, 2H), 10.25 (s, 1H).

### Example 90

### 6-fluoro-N-[1-(4-fluorobenzyl)-3,5-dimethyl-1H-pyrazol-4-yl]-2-methylquinoline-4-carboxamide

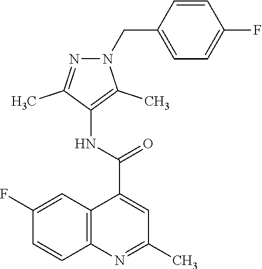 [SMILES: Cc1cc(C(=O)Nc2c(C)nn(Cc3ccc(F)cc3)c2C)c2cc(F)ccc2n1]

In analogy to example 1), 100 mg (0.46 mmol) 1-(4-fluorobenzyl)-3,5-dimethyl-1H-pyrazol-4-amine (intermediate 1C) was stirred with 113 mg (0.55 mmol) 6-fluoro-2-methylquinoline-4-carboxylic acid, 119 μL (0.68 mmol) N,N-diisopropylethylamine and 220 mg (0.68 mmol) TBTU in 5 mL tetrahydrofuran for 3 h at 25° C. The reaction mixture was evaporated and the residue partitioned between ethyl acetate and water. The layers were separated and the aqueous layer was extracted two further times with ethyl acetate. The combined organic layers were washed with brine, dried over sodium sulfate, filtered and evaporated. The residue was dissolved in dichloromethane and under evaporation adsorbed on Isolute® HM-N (Biotage). The isolute was given on a Biotage SNAP cartridge (25 g; KP-Sil) preequilibrated with hexane and purified via column chromatography on silica gel (solvent: hexane/0-100% ethyl acetate). The resulting crude product was again purified via preparative HPLC (method 3) to obtain 75 mg (0.18 mmol, 40%) of the desired title compound.

1H NMR (400 MHz, DMSO d₆): δ (ppm)=2.13 (s, 3H), 2.17 (s, 3H), 2.72 (s, 3H), 5.24 (s, 2H), 7.15-7.28 (m, 4H), 7.67-7.74 (m, 2H), 7.83 (dd, 1H), 8.08 (dd, 1H), 9.92 (s, 1H).

### Example 91

### 6,8-dichloro-N-[1-(3-cyanobenzyl)-3,5-dimethyl-1H-pyrazol-4-yl]-2-(trifluoromethyl)quinoline-4-carboxamide

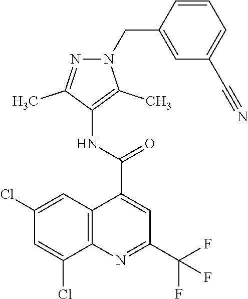 [SMILES: Cc1nn(Cc2cccc(C#N)c2)c(C)c1NC(=O)c1cc(C(F)(F)F)nc2c(Cl)cc(Cl)cc12]

In analogy to example 1), 50 mg (0.22 mmol) 3-[(4-amino-3,5-dimethyl-1H-pyrazol-1-yl)methyl]benzonitrile (intermediate 9C) was stirred with 82 mg (0.27 mmol) 6,8-dichloro-2-(trifluoromethyl)quinoline-4-carboxylic acid (intermediate 11A), 58 μL (0.33 mmol) N,N-diisopropylethylamine and 106 mg (0.33 mmol) TBTU in 5 mL tetrahydrofuran for 24 h at 25° C. to obtain 85 mg (0.16 mmol, 73%) of the desired title compound after preparative HPLC (method 3).

1H NMR (400 MHz, DMSO d₆): δ (ppm)=2.16 (s, 3H), 2.21 (s, 3H), 5.34 (s, 2H), 7.47-7.52 (m, 1H), 7.57-7.63 (m, 2H), 7.76-7.81 (m, 1H), 8.31 (d, 1H), 8.40 (d, 1H), 8.42 (s, 1H), 10.22 (s, 1H).

### Example 92

### N-(1-benzyl-3,5-dimethyl-1H-pyrazol-4-yl)-2, 6-dimethylquinoline-4-carboxamide

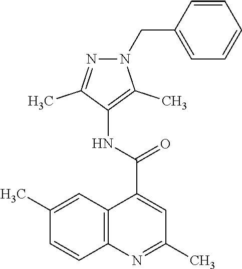 [SMILES: Cc1ccc2nc(C)cc(C(=O)Nc3c(C)nn(Cc4ccccc4)c3C)c2c1]

In analogy to example 1), 50 mg (0.25 mmol) 1-benzyl-3,5-dimethyl-1H-pyrazol-4-amine (intermediate 30C) was stirred with 60 mg (0.30 mmol) 2,6-dimethylquinoline-4-carboxylic acid, 65 μL (0.37 mmol) N,N-diisopropylethylamine and 120 mg (0.37 mmol) TBTU in 5 mL tetrahydrofuran for 3 h at 25° C. to obtain 56 mg (0.15 mmol, 59%) of the desired title compound after preparative HPLC (method 3).

1H NMR (300 MHz, DMSO d₆): δ (ppm)=2.14 (s, 3H), 2.16 (s, 3H), 2.68 (s, 3H), 5.24 (s, 2H), 7.11-7.22 (m, 2H), 7.24-7.40 (m, 3H), 7.55 (s, 1H), 7.60 (dd, 1H), 7.85-7.92 (m, 2H), 9.83 (s, 1H).

### Example 93

### 6,8-dichloro-N-{3,5-dimethyl-1-[3-(trifluoromethoxy)benzyl]-1H-pyrazol-4-yl}-2-(trifluoromethyl)quinoline-4-carboxamide

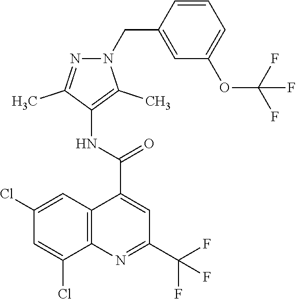 [SMILES: Cc1nn(Cc2cccc(OC(F)(F)F)c2)c(C)c1NC(=O)c1cc(C(F)(F)F)nc2c(Cl)cc(Cl)cc12]

In analogy to example 1), 50 mg (0.18 mmol) 3,5-dimethyl-1-[3-(trifluoromethoxy)benzyl]-1H-pyrazol-4-amine (intermediate 20C) was stirred with 65 mg (0.21 mmol) 6,8-dichloro-2-(trifluoromethyl)quinoline-4-carboxylic acid (intermediate 11A), 46 μL (0.26 mmol) N,N-diisopropylethylamine and 84 mg (0.26 mmol) TBTU in 3 mL tetrahydrofuran for 24 h at 25° C. to obtain 59 mg (0.10 mmol, 58%) of the desired title compound after preparative HPLC (method 3).

1H NMR (300 MHz, DMSO d₆): δ (ppm)=2.15 (s, 3H), 2.19 (s, 3H), 5.34 (s, 2H), 7.14-7.21 (m, 2H), 7.30 (d, 1H), 7.51 (t, 1H), 8.30 (d, 1H), 8.38-8.45 (m, 2H), 10.24 (s, 1H).

### Example 94

### 6,8-dichloro-N-[1-(2-fluorobenzyl)-3,5-dimethyl-1H-pyrazol-4-yl]-2-(trifluoromethyl)quinoline-4-carboxamide

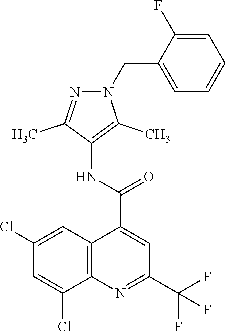 [SMILES: Cc1nn(Cc2ccccc2F)c(C)c1NC(=O)c1cc(C(F)(F)F)nc2c(Cl)cc(Cl)cc12]

In analogy to example 1), 50 mg (0.23 mmol) 1-(2-fluorobenzyl)-3,5-dimethyl-1H-pyrazol-4-amine (intermediate 3C) was stirred with 85 mg (0.27 mmol) 6,8-dichloro-2-(trifluoromethyl)quinoline-4-carboxylic acid (intermediate 11A), 60 μL (0.34 mmol) N,N-diisopropylethylamine and 110 mg (0.34 mmol) TBTU in 5 mL tetrahydrofuran for 24 h at 25° C. to obtain 74 mg (0.14 mmol, 63%) of the desired title compound after preparative HPLC (method 3).

1H NMR (400 MHz, DMSO d₆): δ (ppm)=2.13 (s, 3H), 2.23 (s, 3H), 5.30 (s, 2H), 7.06 (td, 1H), 7.16-7.25 (m, 2H), 7.33-7.41 (m, 1H), 8.30 (d, 1H), 8.40 (d, 1H), 8.42 (s, 1H), 10.21 (s, 1H).

### Example 95

### N-[1-(4-fluorobenzyl)-3,5-dimethyl-1H-pyrazol-4-yl]-3-methoxy-2-methylquinoline-4-carboxamide

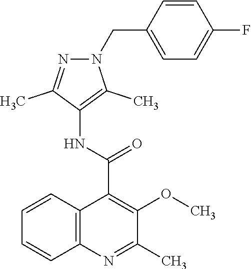 [SMILES: COc1c(C)nc2ccccc2c1C(=O)Nc1c(C)nn(Cc2ccc(F)cc2)c1C]

50 mg (0.23 mmol) 1-(4-fluorobenzyl)-3,5-dimethyl-1H-pyrazol-4-amine (intermediate 1C) were dissolved in 2 mL N,N-dimethylformamide. 64 mg (0.29 mmol) 3-methoxy-2-methylquinoline-4-carboxylic acid (J. Chem. Soc. 1963, p. 491-497), 60 μL (0.34 mmol) N,N-diisopropylethylamine, 218 mg (1.14 mmol) 1-(3-Dimethylaminopropyl)-3-ethylcarbodiimidhydrochlorid and 52 mg (0.34 mmol) 1-Hydroxy-1H-benzotriazole (mono)hydrate were added and the reaction mixture was stirred 24 h at 25° C. The reaction mixture was partitioned between dichloromethane and water. The layers were separated the organic layer was washed with brine, dried over sodium sulfate, filtered and evaporated. The residue was purified via preparative HPLC (method 3) to obtain 42 mg (0.10 mmol, 44%) of the desired title compound.

1H NMR (300 MHz, DMSO d₆): δ (ppm)=2.17 (s, 3H), 2.21 (s, 3H), 2.67 (s, 3H), 3.93 (s, 3H), 5.24 (s, 2H), 7.14-7.30 (m, 4H), 7.57-7.66 (m, 1H), 7.66-7.74 (m, 1H), 7.76-7.82 (m, 1H), 7.98 (d, 1H), 9.92 (s, 1H).

### Example 96

### N-[1-(2,4-difluorobenzyl)-3,5-dimethyl-1H-pyrazol-4-yl]-2,6-dimethylquinoline-4-carboxamide

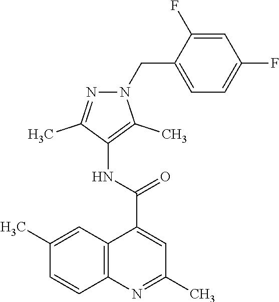 [SMILES: Cc1ccc2nc(C)cc(C(=O)Nc3c(C)nn(Cc4ccc(F)cc4F)c3C)c2c1]

In analogy to example 41), 50 mg (0.21 mmol) 1-(2,4-difluorobenzyl)-3,5-dimethyl-1H-pyrazol-4-amine (intermediate 5C) was stirred with 51 mg (0.25 mmol) 2,6-dimethylquinoline-4-carboxylic acid, 55 μL (0.32 mmol) N,N-diisopropylethylamine and 165 mg (0.32 mmol) PyBOP in 5 mL tetrahydrofuran for 24 h at 25° C. to obtain 34 mg (0.08 mmol, 38%) of the desired title compound after preparative HPLC (method 3).

1H NMR (400 MHz, DMSO d₆): δ (ppm)=2.12 (s, 3H), 2.22 (s, 3H), 2.71 (s, 3H), 5.25 (s, 2H), 7.05-7.22 (m, 2H), 7.25-7.33 (m, 1H), 7.58-7.66 (m, 2H), 7.85-7.94 (m, 2H), 9.87 (s, 1H).

### Example 97

### 6-bromo-N-[1-(3-methoxybenzyl)-3,5-dimethyl-1H-pyrazol-4-yl]-2-(trifluoromethyl)quinoline-4-carboxamide

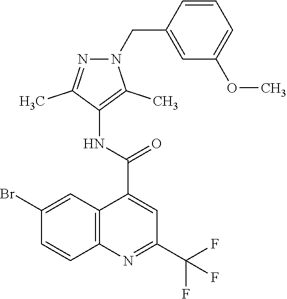 [SMILES: COc1cccc(Cn2nc(C)c(NC(=O)c3cc(C(F)(F)F)nc4ccc(Br)cc34)c2C)c1]

In analogy to example 1), 50 mg (0.21 mmol) 1-(3-methoxybenzyl)-3,5-dimethyl-1H-pyrazol-4-amine (intermediate 12C) was stirred with 83 mg (0.26 mmol) 6-bromo-2-(trifluoromethyl)quinoline-4-carboxylic acid (intermediate 1A), 56 μL (0.32 mmol) N,N-diisopropylethylamine and 104 mg (0.32 mmol) TBTU in 5 mL tetrahydrofuran for 24 h at 25° C. to obtain 77 mg (0.14 mmol, 65%) of the desired title compound after preparative HPLC (method 3).

1H NMR (300 MHz, DMSO d₆): δ (ppm)=2.16 (s, 3H), 2.18 (s, 3H), 3.73 (s, 3H), 5.23 (s, 2H), 6.68-6.75 (m, 2H), 6.82-6.89 (m, 1H), 7.27 (t, 1H), 8.12-8.19 (m, 1H), 8.23 (d, 1H), 8.30 (s, 1H), 8.50 (d, 1H), 10.18 (s, 1H).

### Example 98

### N-[1-(4-fluorobenzyl)-3,5-dimethyl-1H-pyrazol-4-yl]-2,6-bis(trifluoromethyl)quinoline-4-carboxamide

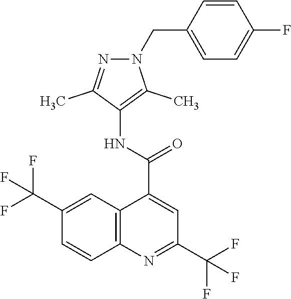 [SMILES: Cc1nn(Cc2ccc(F)cc2)c(C)c1NC(=O)c1cc(C(F)(F)F)nc2ccc(C(F)(F)F)cc12]

In analogy to example 1), 50 mg (0.23 mmol 1-(4-fluorobenzyl)-3,5-dimethyl-1H-pyrazol-4-amine (intermediate 1C) was stirred with 106 mg (0.27 mmol, 80%) 2,6-bis(trifluoromethyl)quinoline-4-carboxylic acid (intermediate 23A), 60 μL (0.34 mmol) N,N-diisopropylethylamine and 110 mg (0.34 mmol) TBTU in 3 mL tetrahydrofuran for 24 h at 25° C. to obtain 59 mg (0.11 mmol, 49%) of the desired title compound after preparative HPLC (method 3).

1H NMR (300 MHz, DMSO d₆): δ (ppm)=2.15 (s, 3H), 2.20 (s, 3H), 5.25 (s, 2H), 7.10-7.30 (m, 4H), 8.28 (dd, 1H), 8.42 (s, 1H), 8.51 (d, 1H), 8.70 (s, 1H), 10.22 (s, 1H).

### Example 99

### 2-chloro-N-[1-(4-fluorobenzyl)-3,5-dimethyl-1H-pyrazol-4-yl]quinoline-4-carboxamide

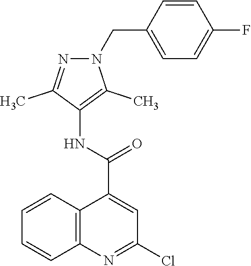 [SMILES: Cc1nn(Cc2ccc(F)cc2)c(C)c1NC(=O)c1cc(Cl)nc2ccccc12]

In analogy to example 1), 100 mg (0.45 mmol) 1-(4-fluorobenzyl)-3,5-dimethyl-1H-pyrazol-4-amine (intermediate 1C) was stirred with 114 mg (0.55 mmol) 2-chloroquinoline-4-carboxylic acid, 119 μL (0.68 mmol) N,N-diisopropylethylamine and 220 mg (0.68 mmol) TBTU in 5 mL tetrahydrofuran for 3 h at 25° C. The reaction mixture was evaporated and the residue partitioned between ethyl acetate and water. The layers were separated and the aqueous layer was extracted two further times with ethyl acetate. The combined organic layers were washed with brine, dried over sodium sulfate, filtered and evaporated. The residue was dissolved in dichloromethane and under evaporation adsorbed on Isolute® HM-N (Biotage). The isolute was given on a Biotage SNAP cartridge (25 g; KP-Sil) preequilibrated with hexane and purified via column chromatography on silica gel (solvent: hexane/0-100% ethyl acetate) to obtain 65 mg (0.15 mmol, 33%) of the desired title compound.

1H NMR (400 MHz, DMSO d₆): δ (ppm)=2.14 (s, 3H), 2.19 (s, 3H), 5.24 (s, 2H), 7.15-7.27 (m, 4H), 7.76 (ddd, 1H), 7.84 (s, 1H), 7.90 (ddd, 1H), 8.05 (d, 1H), 8.14-8.19 (m, 1H), 10.02 (s, 1H).

### Example 100

### 7-bromo-2-cyclopropyl-N-[1-(4-fluorobenzyl)-3,5-dimethyl-1H-pyrazol-4-yl]quinoline-4-carboxamide

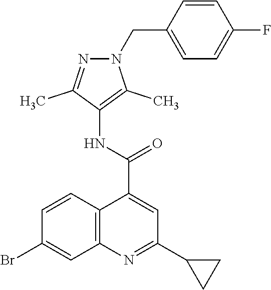 [SMILES: Cc1nn(Cc2ccc(F)cc2)c(C)c1NC(=O)c1cc(C2CC2)nc2cc(Br)ccc12]

In analogy to example 1), 50 mg (0.23 mmol 1-(4-fluorobenzyl)-3,5-dimethyl-1H-pyrazol-4-amine (intermediate 1C) was stirred with 80 mg (0.27 mmol) 7-bromo-2-cyclopropylquinoline-4-carboxylic acid (intermediate 29A), 60 μL (0.34 mmol) N,N-diisopropylethylamine and 110 mg (0.34 mmol) TBTU in 3 mL tetrahydrofuran for 24 h at 25° C. to obtain 75 mg (0.11 mmol, 66%) of the desired title compound after preparative HPLC (method 3).

1H NMR (400 MHz, DMSO d₆): δ (ppm)=1.08-1.19 (m, 4H), 2.12 (s, 3H), 2.16 (s, 3H), 2.36-2.44 (m, 1H), 5.24 (s, 2H), 7.08-7.30 (m, 4H), 7.63-7.77 (m, 2H), 8.04 (d, 1H), 8.11 (d, 1H), 9.91 (s, 1H).

### Example 101

### N-[3,5-dimethyl-1-(3-methylbenzyl)-1H-pyrazol-4-yl]-2,6-dimethylquinoline-4-carboxamide

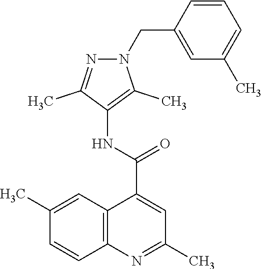

In analogy to example 1), 50 mg (0.23 mmol 3,5-dimethyl-1-(3-methylbenzyl)-1H-pyrazol-4-amine (intermediate 14C) was stirred with 56 mg (0.28 mmol) 2,6-dimethylquinoline-4-carboxylic acid, 61 μL (0.35 mmol) N,N-diisopropylethylamine and 112 mg (0.35 mmol) TBTU in 5 mL tetrahydrofuran for 24 h at 25° C. to obtain 43 mg (0.11 mmol, 46%) of the desired title compound after preparative HPLC (method 3).

1H NMR (400 MHz, DMSO d₆): δ (ppm)=2.14 (s, 3H), 2.17 (s, 3H), 2.29 (s, 3H), 2.69 (s, 3H), 5.20 (s, 2H), 6.96 (d, 1H), 7.04 (s, 1H), 7.10 (d, 1H), 7.24 (t, 1H), 7.56 (s, 1H), 7.61 (dd, 1H), 7.86-7.91 (m, 2H), 9.83 (s, 1H).

### Example 102

### 8-cyano-N-[1-(4-fluorobenzyl)-3,5-dimethyl-1H-pyrazol-4-yl]-6-methyl-2-(trifluoromethyl)quinoline-4-carboxamide

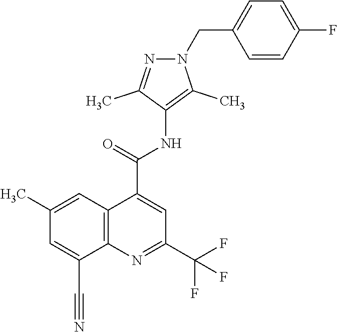 [SMILES: Cc1cc(C#N)c2nc(C(F)(F)F)cc(C(=O)Nc3c(C)nn(Cc4ccc(F)cc4)c3C)c2c1]

In analogy to example 80, 100 mg (0.19 mmol) 8-bromo-N-[1-(4-fluorobenzyl)-3,5-dimethyl-1H-pyrazol-4-yl]-6-methyl-2-(trifluoromethyl)quinoline-4-carboxamide (example 21) in degassed 2.3 mL N,N-dimethylformamide was added 24 mg (0.21 mmol) zinc cyanide and 22 mg (0.02 mmol) tetrakis(triphenylphospine)palladium(0) and the reaction mixture was heated for 10 minutes at 150° C. in a microwave. The reaction suspension was poured on a biphasic mixture of water and ethyl acetate and the aqueous layer was extracted with ethyl acetate two further times. The combined organic layers were washed with brine, dried over sodium sulfate, filtered and evaporated. The residue was dissolved in 2.5 mL N,N-dimethylformamide and purified via preparative HPLC (method 3) to obtain 20 mg (0.04 mmol, 22%) of the desired title compound after drying.

1H NMR (400 MHz, DMSO d₆): δ (ppm)=2.15 (s, 3H), 2.20 (s, 3H), 2.60 (s, 3H), 5.25 (s, 2H), 7.16-7.28 (m, 4H), 8.35 (s, 1H), 8.37 (s, 1H), 8.53 (d, 1H), 10.16 (s, 1H).

### Example 103

### 6-cyano-N-[1-(4-fluorobenzyl)-3,5-dimethyl-1H-pyrazol-4-yl]-2-(trifluoromethyl)quinoline-4-carboxamide

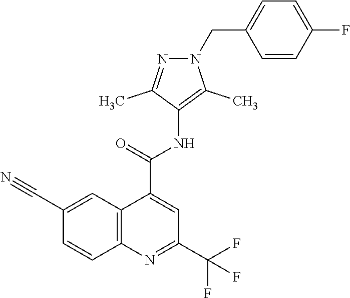 [SMILES: Cc1nn(Cc2ccc(F)cc2)c(C)c1NC(=O)c1cc(C(F)(F)F)nc2ccc(C#N)cc12]

In analogy to example 80, 100 mg (0.19 mmol) 6-bromo-N-[1-(4-fluorobenzyl)-3,5-dimethyl-1H-pyrazol-4-yl]-2-(trifluoromethyl)quinoline-4-carboxamide (example 9) in degassed 2.4 mL N,N-dimethylformamide was added 25 mg (0.21 mmol) zinc cyanide and 22 mg (0.02 mmol) tetrakis(triphenylphospine)palladium(0) and the reaction mixture was heated for 10 minutes at 150° C. in a microwave. The reaction suspension was poured on a biphasic mixture of water and ethyl acetate and the aqueous layer was extracted with ethyl acetate two further times. The combined organic layers were washed with brine, dried over sodium sulfate, filtered and evaporated. The residue was dissolved in 2.5 mL N,N-dimethylformamide and purified via preparative HPLC (method 3) to obtain 45 mg (0.10 mmol, 50%) of the desired title compound after drying.

1H NMR (400 MHz, DMSO d₆): δ (ppm)=2.16 (s, 3H), 2.20 (s, 3H), 5.26 (s, 2H), 7.15-7.28 (m, 4H), 8.31 (dd, 1H), 8.40 (s, 1H), 8.45 (d, 1H), 8.83 (d, 1H), 10.22 (s, 1H).

### Example 104

### 6,8-dichloro-N-[1-(3-fluorobenzyl)-3,5-dimethyl-1H-pyrazol-4-yl]-2-(trifluoromethyl)quinoline-4-carboxamide

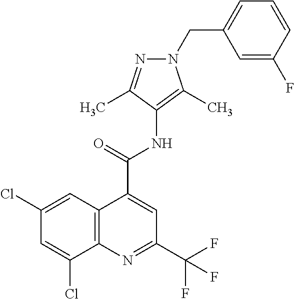 [SMILES: Cc1nn(Cc2cccc(F)c2)c(C)c1NC(=O)c1cc(C(F)(F)F)nc2c(Cl)cc(Cl)cc12]

In analogy to example 1), 50 mg (0.23 mmol 1-(3-fluorobenzyl)-3,5-dimethyl-1H-pyrazol-4-amine (intermediate 2C) was stirred with 85 mg (0.27 mmol) 6,8-dichloro-2-(trifluoromethyl)quinoline-4-carboxylic acid (intermediate 11A), 60 μL (0.34 mmol) N,N-diisopropylethylamine and 110 mg (0.34 mmol) TBTU in 5 mL tetrahydrofuran for 24 h at 25° C. to obtain 77 mg (0.15 mmol, 65%) of the desired title compound after preparative HPLC (method 3).

1H NMR (400 MHz, DMSO d₆): δ (ppm)=2.16 (s, 3H), 2.19 (s, 3H), 5.29 (s, 2H), 6.90-6.98 (m, 1H), 7.01 (d, 1H), 7.13 (td, 1H), 7.41 (td, 1H), 8.31 (d, 1H), 8.40 (d, 1H), 8.42 (s, 1H), 10.22 (s, 1H).

### Example 105

### N-[1-(4-fluorobenzyl)-3,5-dimethyl-1H-pyrazol-4-yl]-2-methylquinoline-4-carboxamide

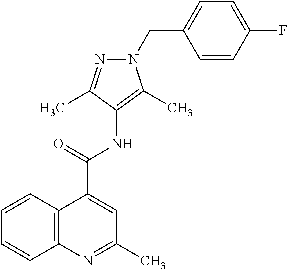 [SMILES: Cc1cc(C(=O)Nc2c(C)nn(Cc3ccc(F)cc3)c2C)c2ccccc2n1]

In analogy to example 1), 150 mg (0.68 mmol) 1-(4-fluorobenzyl)-3,5-dimethyl-1H-pyrazol-4-amine (intermediate 1C) was stirred with 154 mg (0.82 mmol) 2-methylquinoline-4-carboxylic acid, 179 μL (1.03 mmol) N,N-diisopropylethylamine and 329 mg (1.03 mmol) TBTU in 5 mL tetrahydrofuran for 2 h at 25° C. The reaction mixture was evaporated and the residue partitioned between ethyl acetate and water. The layers were separated and the aqueous layer was extracted two further times with ethyl acetate. The combined organic layers were washed with brine, dried over sodium sulfate, filtered and evaporated. The residue was dissolved in dichloromethane and under evaporation adsorbed on Isolute® HM-N (Biotage). The isolute was given on a Biotage SNAP cartridge (25 g; KP-Sil) preequilibrated with hexane and purified via column chromatography on silica gel (solvent: ethyl acetate/0-20% methanol) to obtain 237 mg (0.61 mmol, 89%) of the desired title compound.

1H NMR (300 MHz, CDCl₃): δ (ppm)=2.23 (s, 3H), 2.31 (s, 3H), 2.81 (s, 3H), 5.24 (s, 2H), 6.98-7.08 (m, 2H), 7.11-7.23 (m, 3H), 7.48 (s, 1H), 7.55-7.64 (m, 1H), 7.72-7.81 (m, 1H), 8.07-8.15 (m, 1H), 8.25 (d, 1H).

### Example 106

### 6,7-dichloro-N-[1-(4-fluorobenzyl)-3,5-dimethyl-1H-pyrazol-4-yl]-2-(trifluoromethyl)quinoline-4-carboxamide

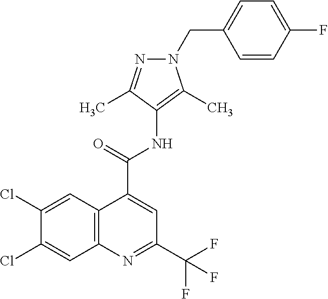 [SMILES: Cc1nn(Cc2ccc(F)cc2)c(C)c1NC(=O)c1cc(C(F)(F)F)nc2cc(Cl)c(Cl)cc12]

In analogy to example 1), 50 mg (0.23 mmol) 1-(4-fluorobenzyl)-3,5-dimethyl-1H-pyrazol-4-amine (intermediate 1C) was stirred with 84 mg (0.27 mmol) of a mixture of 5,6-dichloro-2-(trifluoromethyl)quinoline-4-carboxylic acid and 6,7-dichloro-2-(trifluoromethyl)quinoline-4-carboxylic acid (3:1) (intermediate 15A), 60 μL (0.34 mmol) N,N-diisopropylethylamine and 109 mg (0.34 mmol) TBTU in 5 mL tetrahydrofuran for 24 h at 25° C. to obtain 23 mg (0.04 mmol, 20%) of the desired title compound after preparative HPLC (method 6). Furthermore 32 mg (0.06 mmol, 27%) of 5,6-dichloro-N-[1-(4-fluorobenzyl)-3,5-dimethyl-1H-pyrazol-4-yl]-2-(trifluoromethyl)quinoline-4-carboxamide (example 22) were isolated after preparative HPLC.

1H NMR (400 MHz, DMSO d₆): δ (ppm)=2.14 (s, 3H), 2.19 (s, 3H), 5.25 (s, 2H), 7.13-7.28 (m, 4H), 8.33 (s, 1H), 8.57 (s, 1H), 8.65 (s, 1H), 10.19 (s, 1H).

### Example 107

### N-{3,5-dimethyl-1-[4-(trifluoromethyl)benzyl]-1H-pyrazol-4-yl}-2, 6-dimethylquinoline-4-carboxamide

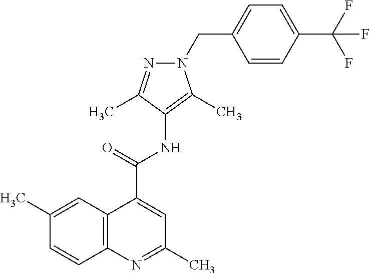 [SMILES: Cc1ccc2nc(C)cc(C(=O)Nc3c(C)nn(Cc4ccc(C(F)(F)F)cc4)c3C)c2c1]

In analogy to example 1), 50 mg (0.19 mmol) 3,5-dimethyl-1-[4-(trifluoromethyl)benzyl]-1H-pyrazol-4-amine (intermediate 22C) was stirred with 44 mg (0.22 mmol) 2,6-dimethylquinoline-4-carboxylic acid, 49 μL (0.28 mmol) N,N-diisopropylethylamine and 89 mg (0.28 mmol) TBTU in 5 mL tetrahydrofuran for 3 h at 25° C. to obtain 58 mg (0.12 mmol, 67%) of the desired title compound after preparative HPLC (method 3).

1H NMR (400 MHz, DMSO d₆): δ (ppm)=2.14 (s, 3H), 2.17 (s, 3H), 2.68 (s, 3H), 5.36 (s, 2H), 7.37 (d, 2H), 7.56 (s, 1H), 7.60 (dd, 1H), 7.74 (d, 2H), 7.85-7.91 (m, 2H), 9.86 (s, 1H).

### Example 108

### 2-cyclopropyl-N-[1-(4-fluorobenzyl)-3,5-dimethyl-1H-pyrazol-4-yl]-8-(trifluoromethyl)quinoline-4-carboxamide

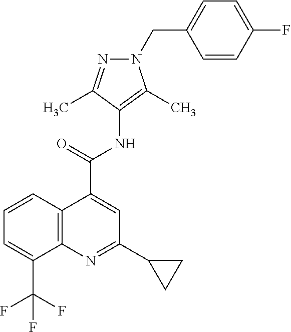 [SMILES: Cc1nn(Cc2ccc(F)cc2)c(C)c1NC(=O)c1cc(C2CC2)nc2c(C(F)(F)F)cccc12]

In analogy to example 1), 50 mg (0.23 mmol) 1-(4-fluorobenzyl)-3,5-dimethyl-1H-pyrazol-4-amine (intermediate 1C) was stirred with 77 mg (0.27 mmol) 2-cyclopropyl-8-(trifluoromethyl)quinoline-4-carboxylic acid (intermediate 36A), 60 μL (0.34 mmol) N,N-diisopropylethylamine and 110 mg (0.34 mmol) TBTU in 5 mL tetrahydrofuran for 24 h at 25° C. to obtain 92 mg (0.18 mmol, 80%) of the desired title compound after preparative HPLC (method 3).

1H NMR (400 MHz, DMSO d): δ (ppm)=1.11-1.22 (m, 4H), 2.15 (s, 3H), 2.19 (s, 3H), 2.40-2.47 (m, 1H), 5.25 (s, 2H), 7.13-7.29 (m, 4H), 7.69 (t, 1H), 7.84 (s, 1H), 8.16 (d, 1H), 8.35 (d, 1H), 9.94 (s, 1H).

### Example 109

### N-[1-(3-methoxybenzyl)-3,5-dimethyl-1H-pyrazol-4-yl]-2,6-dimethylquinoline-4-carboxamide

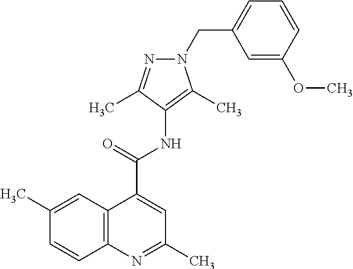

In analogy to example 1), 50 mg (0.22 mmol) 1-(3-methoxybenzyl)-3,5-dimethyl-1H-pyrazol-4-amine (intermediate 12C) was stirred with 52 mg (0.26 mmol) 2,6-dimethylquinoline-4-carboxylic acid, 56 μL (0.32 mmol) N,N-diisopropylethylamine and 104 mg (0.32 mmol) TBTU in 5 mL tetrahydrofuran for 24 h at 25° C. to obtain 52 mg (0.12 mmol, 57%) of the desired title compound after preparative HPLC (method 3).

1H NMR (400 MHz, DMSO d₆): δ (ppm)=2.15 (s, 3H), 2.17 (s, 3H), 2.69 (s, 3H), 3.73 (s, 3H), 5.22 (s, 2H), 6.70-6.76 (m, 2H), 6.83-6.89 (m, 1H), 7.27 (t, 1H), 7.57 (s, 1H), 7.61 (dd, 1H), 7.86-7.92 (m, 2H), 9.84 (s, 1H).

### Example 110

### N-[3,5-dimethyl-1-(pyridin-2-ylmethyl)-1H-pyrazol-4-yl]-2-methoxyquinoline-4-carboxamide

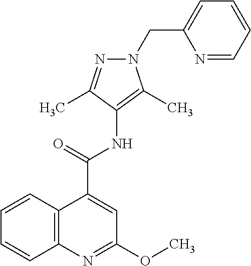 [SMILES: COc1cc(C(=O)Nc2c(C)nn(Cc3ccccn3)c2C)c2ccccc2n1]

In analogy to example 1), 50 mg (0.25 mmol) 3,5-dimethyl-1-(pyridin-2-ylmethyl)-1H-pyrazol-4-amine (intermediate 18C) was stirred with 60 mg (0.30 mmol) 2-methoxyquinoline-4-carboxylic acid, 65 μL (0.37 mmol) N,N-diisopropylethylamine and 119 mg (0.37 mmol) TBTU in 5 mL tetrahydrofuran for 24 h at 25° C. to obtain 64 mg (0.16 mmol, 66%) of the desired title compound after preparative HPLC (method 3).

1H NMR (400 MHz, DMSO d₆): δ (ppm)=2.12 (s, 3H), 2.21 (s, 3H), 4.05 (s, 3H), 5.33 (s, 2H), 7.03 (d, 1H), 7.23 (s, 1H), 7.29-7.34 (m, 1H), 7.52 (td, 1H), 7.74 (td, 1H), 7.79 (td, 1H), 7.86 (d, 1H) 8.06 (d, 1H), 8.52-8.56 (m, 1H), 9.91 (s, 1H).

### Example 111

### N-[3,5-dimethyl-1-(2-methylbenzyl)-1H-pyrazol-4-yl]-2,6-dimethylquinoline-4-carboxamide

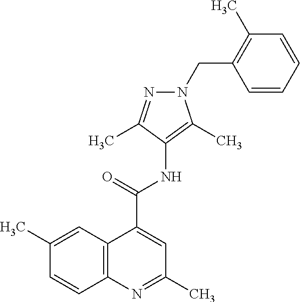 [SMILES: Cc1ccc2nc(C)cc(C(=O)Nc3c(C)nn(Cc4ccccc4C)c3C)c2c1]

In analogy to example 1), 50 mg (0.23 mmol) 3,5-dimethyl-1-(2-methylbenzyl)-1H-pyrazol-4-amine (intermediate 15C) was stirred with 56 mg (0.28 mmol) 2,6-dimethylquinoline-4-carboxylic acid, 61 μL (0.35 mmol) N,N-diisopropylethylamine and 112 mg (0.35 mmol) TBTU in 5 mL tetrahydrofuran for 24 h at 25° C. The reaction mixture was evaporated and acetonitrile was added. The precipitated product was isolated by filtration, washed with acetonitrile and dried in high vacuum to obtain 18 mg (0.04 mmol, 18%) of the desired title compound after preparative HPLC (method 3).

1H NMR (300 MHz, DMSO d₆): δ (ppm)=2.15 (s, 6H), 2.35 (s, 3H), 2.70 (s, 3H), 5.25 (s, 2H), 6.61 (d, 1H), 7.09-7.24 (m, 3H), 7.57-7.66 (m, 2H), 7.85-7.94 (m, 2H), 9.89 (s, 1H).

### Example 112

### N-[1-(2-fluorobenzyl)-3,5-dimethyl-1H-pyrazol-4-yl]-2,6-dimethylquinoline-4-carboxamide

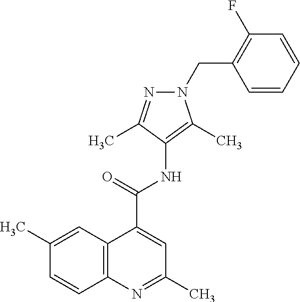 [SMILES: Cc1ccc2nc(C)cc(C(=O)Nc3c(C)nn(Cc4ccccc4F)c3C)c2c1]

In analogy to example 1), 50 mg (0.23 mmol) 1-(2-fluorobenzyl)-3,5-dimethyl-1H-pyrazol-4-amine (intermediate 3C) was stirred with 55 mg (0.27 mmol) 2,6-dimethylquinoline-4-carboxylic acid, 60 μL (0.34 mmol) N,N-diisopropylethylamine and 110 mg (0.34 mmol) TBTU in 5 mL tetrahydrofuran for 24 h at 25° C. to obtain 28 mg (0.06 mmol, 30%) of the desired title compound after preparative HPLC (method 3).

1H NMR (300 MHz, DMSO d₆): δ (ppm)=2.13 (s, 3H), 2.22 (s, 3H), 2.70 (s, 3H), 5.29 (s, 2H), 7.03-7.11 (m, 1H), 7.14-7.30 (m, 2H), 7.33-7.43 (m, 1H), 7.57 (s, 1H), 7.61 (dd, 1H), 7.85-7.93 (m, 2H), 9.85 (s, 1H).

### Example 113

### 6-bromo-N-{3,5-dimethyl-1-[3-(trifluoromethoxy)benzyl]-1H-pyrazol-4-yl}-2-(trifluoromethyl)quinoline-4-carboxamide

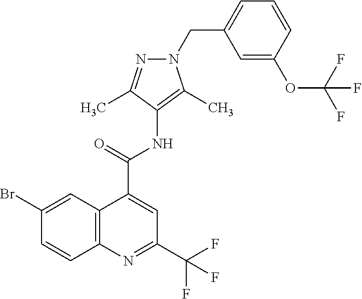 [SMILES: Cc1nn(Cc2cccc(OC(F)(F)F)c2)c(C)c1NC(=O)c1cc(C(F)(F)F)nc2ccc(Br)cc12]

In analogy to example 1), 100 mg (0.18 mmol, 50%) 3,5-dimethyl-1-[3-(trifluoromethoxy)benzyl]-1H-pyrazol-4-amine (intermediate 20C) was stirred with 67 mg (0.21 mmol) 6-bromo-2-(trifluoromethyl)quinoline-4-carboxylic acid (intermediate 1A), 46 μL (0.26 mmol) N,N-diisopropylethylamine and 84 mg (0.26 mmol) TBTU in 3 mL tetrahydrofuran for 24 h at 25° C. to obtain 76 mg (0.13 mmol, 73%) of the desired title compound after preparative HPLC (method 3).

1H NMR (300 MHz, DMSO d₆): δ (ppm)=2.16 (s, 3H), 2.20 (s, 3H), 5.34 (s, 2H), 7.13-7.22 (m, 2H), 7.32 (s, 1H), 7.47-7.56 (m, 1H), 8.11-8.19 (m, 1H), 8.23 (d, 1H), 8.31 (s, 1H), 8.50 (d, 1H), 10.20 (s, 1H).

### Example 114

### N-[1-(2-cyanobenzyl)-3,5-dimethyl-1H-pyrazol-4-yl]-2,6-dimethylquinoline-4-carboxamide

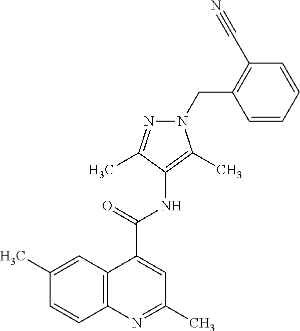 [SMILES: Cc1ccc2nc(C)cc(C(=O)Nc3c(C)nn(Cc4ccccc4C#N)c3C)c2c1]

In analogy to example 1), 50 mg (0.22 mmol) 2-[(4-amino-3,5-dimethyl-1H-pyrazol-1-yl)methyl]benzonitrile (intermediate 10C) was stirred with 53 mg (0.27 mmol) 2,6-dimethylquinoline-4-carboxylic acid, 58 μL (0.33 mmol) N,N-diisopropylethylamine and 106 mg (0.33 mmol) TBTU in 5 mL tetrahydrofuran for 24 h at 25° C. to obtain 54 mg (0.13 mmol, 59%) of the desired title compound after preparative HPLC (method 3).

1H NMR (400 MHz, DMSO d₆): δ (ppm)=2.13 (s, 3H), 2.25 (s, 3H), 2.70 (s, 3H), 5.44 (s, 2H), 7.11 (d, 1H), 7.50-7.56 (m, 1H), 7.58 (s, 1H), 7.61 (dd, 1H), 7.72 (td, 1H), 7.87-7.93 (m, 3H), 9.90 (s, 1H).

### Example 115

### N-{3,5-dimethyl-1-[3-(trifluoromethoxy)benzyl]-1H-pyrazol-4-yl}-2-methoxyquinoline-4-carboxamide

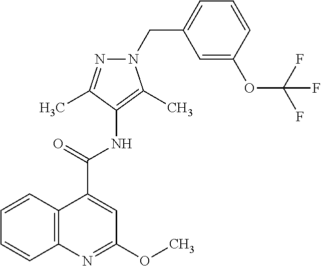 [SMILES: COc1cc(C(=O)Nc2c(C)nn(Cc3cccc(OC(F)(F)F)c3)c2C)c2ccccc2n1]

In analogy to example 1), 100 mg (0.18 mmol, 50%) 3,5-dimethyl-1-[3-(trifluoromethoxy)benzyl]-1H-pyrazol-4-amine (intermediate 20C) was stirred with 43 mg (0.21 mmol) 2-methoxyquinoline-4-carboxylic acid, 46 μL (0.26 mmol) N,N-diisopropylethylamine and 84 mg (0.26 mmol) TBTU in 3 mL tetrahydrofuran for 24 h at 25° C. to obtain 68 mg (0.14 mmol, 81%) of the desired title compound after preparative HPLC (method 3).

1H NMR (300 MHz, DMSO d₆): δ (ppm)=2.14 (s, 3H), 2.17 (s, 3H), 4.04 (s, 3H), 5.33 (s, 2H), 7.13-7.20 (m, 2H), 7.23 (s, 1H), 7.30 (d, 1H), 7.46-7.55 (m, 2H), 7.69-7.77 (m, 1H), 7.86 (d, 1H), 8.05 (d, 1H), 9.92 (s, 1H).

### Example 116

### 6-bromo-N-{3,5-dimethyl-1-[(3-methylpyridin-2-yl)methyl]-1H-pyrazol-4-yl}-2-(trifluoromethyl)quinoline-4-carboxamide

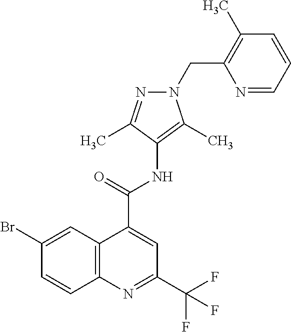 [SMILES: Cc1cccnc1Cn1nc(C)c(NC(=O)c2cc(C(F)(F)F)nc3ccc(Br)cc23)c1C]

In analogy to example 1), 50 mg (0.23 mmol) 3,5-dimethyl-1-[(3-methylpyridin-2-yl)methyl]-1H-pyrazol-4-amine (intermediate 36C) was stirred with 89 mg (0.28 mmol) 6-bromo-2-(trifluoromethyl)quinoline-4-carboxylic acid (intermediate 1A), 60 μL (0.35 mmol) N,N-diisopropylethylamine and 111 mg (0.35 mmol) TBTU in 5 mL tetrahydrofuran for 24 h at 25° C. The reaction mixture was evaporated and dissolved in 2.5 mL N,N-dimethylformamide, whereupon the desired product precipitated out. The suspension was filtered, and the precipitate was washed with tetrahydrofuran and dried in high vacuum to obtain 11 mg (0.02 mmol, 9%) of the desired title compound.

1H NMR (400 MHz, DMSO d₆): δ (ppm)=2.15 (s, 3H), 2.24 (s, 3H), 2.47 (s, 3H), 5.29 (s, 2H), 6.73 (d, 1H), 7.17 (d, 1H), 7.67 (t, 1H), 8.15 (dd, 1H), 8.23 (d, 1H), 8.30 (s, 1H), 8.52 (d, 1H), 10.19 (s, 1H).

### Example 117

### N-{3,5-dimethyl-1-[3-(trifluoromethoxy)benzyl]-1H-pyrazol-4-yl}-2,6-dimethylquinoline-4-carboxamide

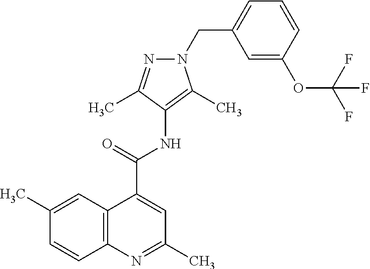 [SMILES: Cc1ccc2nc(C)cc(C(=O)Nc3c(C)nn(Cc4cccc(OC(F)(F)F)c4)c3C)c2c1]

In analogy to example 1), 100 mg (0.18 mmol, 50%) 3,5-dimethyl-1-[3-(trifluoromethoxy)benzyl]-1H-pyrazol-4-amine (intermediate 20C) was stirred with 42 mg (0.21 mmol) 2,6-dimethylquinoline-4-carboxylic acid, 46 μL (0.26 mmol) N,N-diisopropylethylamine and 84 mg (0.26 mmol) TBTU in 3 mL tetrahydrofuran for 24 h at 25° C. to obtain 65 mg (0.14 mmol, 79%) of the desired title compound after preparative HPLC (method 3).

1H NMR (400 MHz, DMSO d₆): δ (ppm)=2.15 (s, 3H), 2.18 (s, 3H), 2.69 (s, 3H), 5.33 (s, 2H), 7.15-7.21 (m, 2H), 7.30 (d, 1H), 7.51 (t, 1H), 7.55-7.63 (m, 2H), 7.86-7.92 (m, 2H), 9.86 (s, 1H).

### Example 118

### 6-bromo-N-[1-(2-cyanobenzyl)-3,5-dimethyl-1H-pyrazol-4-yl]-2-(propan-2-yl)quinoline-4-carboxamide

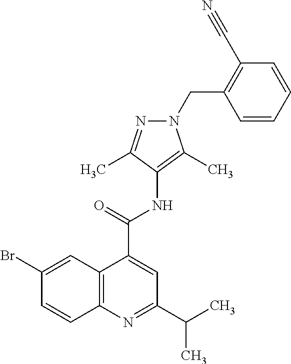 [SMILES: Cc1nn(Cc2ccccc2C#N)c(C)c1NC(=O)c1cc(C(C)C)nc2ccc(Br)cc12]

To a solution of 75 mg (0.26 mmol) 2-[(4-amino-3,5-dimethyl-1H-pyrazol-1-yl)methyl]benzonitrile (intermediate 10C) in 2.1 mL DMSO was added 145 mg (0.31 mmol) HATU, 67 μL N,N-diisopropytethylamine and 69 mg (0.31 mmol) 6-bromo-2-isopropylquinoline-4-carboxylic acid. The reaction mixture was stirred for 20 hours at 25° C. This mixture was directly purified via preparative HPLC (method 4) to obtain 115 mg (85%) of the desired title compound.

¹H-NMR (400 MHz, DMSO d₆) δ (ppm)=1.36 (d, 6H), 2.12 (s, 3H), 2.24 (s, 3H), 3.28 (q, 1H), 5.43 (s, 2H), 7.09 (d, 1H), 7.49-7.55 (m, 1H), 7.71 (td, 1H), 7.76 (s, 1H), 7.87-7.93 (m, 2H), 7.95-8.00 (m, 1H), 8.33 (d, 1H), 10.00 (s, 1H).

### Example 119

### 6-bromo-N⁴-[1-(4-fluorobenzyl)-3,5-dimethyl-1H-pyrazol-4-yl]quinoline-2,4-dicarboxamide

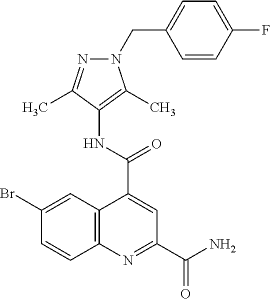 [SMILES: Cc1nn(Cc2ccc(F)cc2)c(C)c1NC(=O)c1cc(C(N)=O)nc2ccc(Br)cc12]

In analogy to example 118), 85.4 mg (0.39 mmol) 1-(4-fluorobenzyl)-3,5-dimethyl-1H-pyrazol-4-amine (intermediate 1C) and 106 mg (0.36 mmol) 6-bromo-2-carbamoylquinoline-4-carboxylic acid (intermediate 2A) were reacted to give after purification via preparative HPLC (method 3) 26 mg (15%) of the desired title compound.

¹H-NMR (300 MHz, DMSO d₆) δ (ppm)=2.13 (s, 3H), 2.17 (s, 3H), 5.24 (s, 2H), 7.14-7.30 (m, 4H), 7.94 (s, 1H), 8.04-8.11 (m, 1H), 8.14 (d, 1H), 8.35 (s, 1H), 8.42 (s, 1H), 8.47 (d, 1H), 10.16 (s, 1H).

### Example 120

### 6,7-difluoro-N⁴-[1-(4-fluorobenzyl)-3,5-dimethyl-1H-pyrazol-4-yl]quinoline-2,4-dicarboxamide

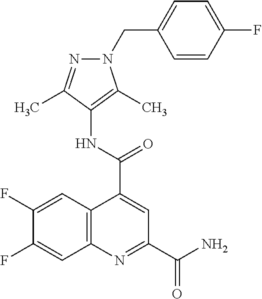 [SMILES: Cc1nn(Cc2ccc(F)cc2)c(C)c1NC(=O)c1cc(C(N)=O)nc2cc(F)c(F)cc12]

In analogy to example 118), 49.6 mg (0.23 mmol) 1-(4-fluorobenzyl)-3,5-dimethyl-1H-pyrazol-4-amine (intermediate 1C) and 190 mg (0.19 mmol, purity 25%) 2-carbamoyl-6,7-difluoroquinoline-4-carboxylic acid (intermediate 3A) were reacted to give after purification via preparative HPLC (method 4) 6.4 mg (7%) of the desired title compound.

¹H-NMR (300 MHz, DMSO d₆) δ (ppm)=2.12 (s, 3H), 2.16 (s, 3H), 5.24 (s, 2H), 7.12-7.30 (m, 4H), 7.96 (br. s., 1H), 8.11-8.29 (m, 2H), 8.32-8.42 (m, 2H), 10.17 (s, 1H).

### Example 121

### 6-chloro-7-fluoro-N-[1-(4-fluorobenzyl)-3,5-dimethyl-1H-pyrazol-4-yl]-2-(trifluoromethyl)quinoline-4-carboxamide

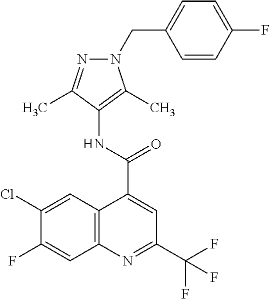 [SMILES: Cc1nn(Cc2ccc(F)cc2)c(C)c1NC(=O)c1cc(C(F)(F)F)nc2cc(F)c(Cl)cc12]

In analogy to example 118), 110 mg (0.50 mmol) 1-(4-fluorobenzyl)-3,5-dimethyl-1H-pyrazol-4-amine (intermediate 1C) and 150 mg (0.42 mmol, purity 825%) 6-chloro-7-fluoro-2-(trifluoromethyl)quinoline-4-carboxylic acid (intermediate 10A) were reacted to give after purification via preparative HPLC (method 3) 87 mg (40%) of the desired title compound.

¹H-NMR (300 MHz, DMSO d₆) δ (ppm)=2.13 (s, 3H), 2.18 (s, 3H), 5.24 (s, 2H), 7.13-7.28 (m, 4H), 8.30 (s, 1H), 8.36 (d, 1H), 8.52 (d, 1H), 10.20 (s, 1H).

### Example 122

### N⁴-[1-(4-fluorobenzyl)-3,5-dimethyl-1H-pyrazol-4-yl]quinoline-2,4-dicarboxamide

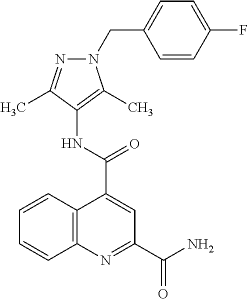 [SMILES: Cc1nn(Cc2ccc(F)cc2)c(C)c1NC(=O)c1cc(C(N)=O)nc2ccccc12]

In analogy to example 118), 121 mg (0.56 mmol) 1-(4-fluorobenzyl)-3,5-dimethyl-1H-pyrazol-4-amine (intermediate 1C) and 100 mg (0.42 mmol, purity 82%) 2-carbamoylquinoline-4-carboxylic acid (intermediate 4A) were reacted to give after purification via preparative HPLC (method 3) 64 mg (30%) of the desired title compound.

¹H-NMR (300 MHz, DMSO d₆) δ (ppm)=2.14 (s, 3H), 2.18 (s, 3H), 5.24 (s, 2H), 7.13-7.29 (m, 4H), 7.76-7.85 (m, 1H), 7.88-7.97 (m, 2H), 8.16-8.29 (m, 3H), 8.39 (br. s., 1H), 10.06 (s, 1H).

### Example 123

### 6-bromo-2-cyclopropyl-N-[1-(3,4-difluorobenzyl)-3,5-dimethyl-1H-pyrazol-4-yl]quinoline-4-carboxamide

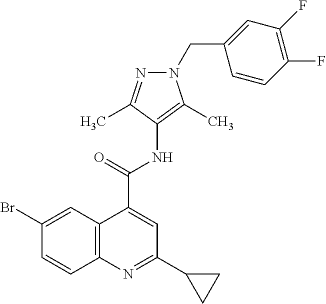 [SMILES: Cc1nn(Cc2ccc(F)c(F)c2)c(C)c1NC(=O)c1cc(C2CC2)nc2ccc(Br)cc12]

In analogy to example 118), 97 mg (0.41 mmol) 1-(3,4-difluorobenzyl)-3,5-dimethyl-1H-pyrazol-4-amine (intermediate 4C) and 100 mg (0.34 mmol) 6-bromo-2-cyclopropylquinoline-4-carboxylic acid were reacted to give after purification via preparative HPLC (method 3) 103 mg (56%) of the desired title compound.

¹H-NMR (300 MHz, DMSO d₆) δ (ppm)=1.07-1.20 (m, 4H), 2.13 (s, 3H), 2.17 (s, 3H), 2.34-2.45 (m, 1H), 5.25 (s, 2H), 7.01 (dd, 1H), 7.17-7.28 (m, 1H), 7.43 (dt, 1H), 7.72 (s, 1H), 7.86 (d, 2H), 8.28 (s, 1H), 9.98 (s, 1H).

### Example 124

### 6-bromo-7-fluoro-N-[1-(4-fluorobenzyl)-3,5-dimethyl-1H-pyrazol-4-yl]-2-(trifluoromethyl)quinoline-4-carboxamide

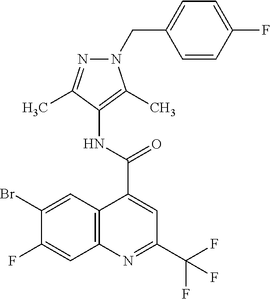 [SMILES: Cc1nn(Cc2ccc(F)cc2)c(C)c1NC(=O)c1cc(C(F)(F)F)nc2cc(F)c(Br)cc12]

In analogy to example 118), 93.4 mg (0.43 mmol) 1-(4-fluorobenzyl)-3,5-dimethyl-1H-pyrazol-4-amine (intermediate 1C) and 150 mg (0.36 mmol, purity 80%) 6-bromo-7-fluoro-2-(trifluoromethyl)quinoline-4-carboxylic acid (intermediate 9A) were reacted to give after purification via preparative HPLC (method 4) 89.8 mg (45%) of the desired title compound.

¹H-NMR (300 MHz, DMSO d₆) δ (ppm)=2.14 (s, 3H), 2.18 (s, 3H), 5.24 (s, 2H), 7.12-7.28 (m, 4H), 8.25-8.34 (m, 2H), 8.67 (d, 1H), 10.20 (s, 1H).

### Example 125

### 6-chloro-2-cyclopropyl-N-[1-(4-fluorobenzyl)-3,5-dimethyl-1H-pyrazol-4-yl]quinoline-4-carboxamide

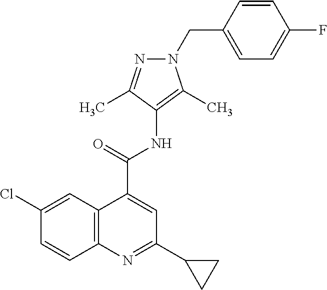 [SMILES: Cc1nn(Cc2ccc(F)cc2)c(C)c1NC(=O)c1cc(C2CC2)nc2ccc(Cl)cc12]

In analogy to example 118), 73 mg (0.33 mmol) 1-(4-fluorobenzyl)-3,5-dimethyl-1H-pyrazol-4-amine (intermediate 1C) and 75 mg (0.30 mmol, purity 80%) 6-chloro-2-cyclopropylquinoline-4-carboxylic acid (intermediate 24A) were reacted to give after purification via preparative HPLC (method 3) 71 mg (48%) of the desired title compound.

¹H-NMR (400 MHz, DMSO d₆) δ (ppm)=1.08-1.18 (m, 4H), 2.12 (s, 3H), 2.16 (s, 3H), 2.35-2.43 (m, 1H), 5.23 (s, 2H), 7.15-7.26 (m, 4H), 7.72 (s, 1H), 7.75 (dd, 1H), 7.93 (d, 1H), 8.12 (d, 1H), 9.94 (s, 1H).

### Example 126

### N⁴-[1-(4-cyanobenzyl)-5-methyl-3-(trifluoromethyl)-1H-pyrazol-4-yl]quinoline-2,4-dicarboxamide

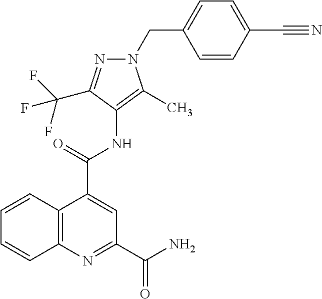 [SMILES: Cc1c(NC(=O)c2cc(C(N)=O)nc3ccccc23)c(C(F)(F)F)nn1Cc1ccc(C#N)cc1]

In analogy to example 118), 109 mg (0.39 mmol) 4-{[4-amino-5-methyl-3-(trifluoromethyl)-1H-pyrazol-1-yl]methyl}benzonitrile (intermediate 26C) and 70 mg (0.32 mmol) 2-carbamoylquinoline-4-carboxylic acid (intermediate 4A) were reacted to give after purification via preparative HPLC (method 4) 42 mg (26%) of the desired title compound.

¹H-NMR (300 MHz, DMSO d₆) δ (ppm)=2.27 (s, 3H), 5.61 (s, 2H), 7.38 (d, 2H), 7.77-7.86 (m, 1H), 7.86-7.99 (m, 4H), 8.20 (dd, 2H), 8.26 (s, 1H), 8.40 (s, 1H), 10.43 (s, 1H).

### Example 127

### 6,8-dichloro-N⁴-[1-(4-fluorobenzyl)-3,5-dimethyl-1H-pyrazol-4-yl]quinoline-2,4-dicarboxamide

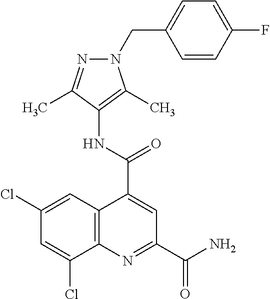 [SMILES: Cc1nn(Cc2ccc(F)cc2)c(C)c1NC(=O)c1cc(C(N)=O)nc2c(Cl)cc(Cl)cc12]

In analogy to example 118), 120 mg (0.55 mmol) 1-(4-fluorobenzyl)-3,5-dimethyl-1H-pyrazol-4-amine (intermediate 1C) and 145 mg (0.46 mmol, purity 90%) 2-carbamoyl-6,8-dichloroquinoline-4-carboxylic acid (intermediate 5A) were reacted to give after purification via preparative HPLC (method 3) 7.6 mg (3%) of the desired title compound.

¹H-NMR (300 MHz, DMSO d₆) δ (ppm)=2.12 (s, 3H), 2.17 (s, 3H), 5.24 (s, 2H), 7.13-7.29 (m, 4H), 8.13 (d, 2H), 8.24-8.28 (m, 1H), 8.28-8.32 (m, 1H), 8.44 (s, 1H), 10.23 (s, 1H).

### Example 128

### N⁴-[1-(3,4-difluorobenzyl)-5-methyl-3-(trifluoromethyl)-1H-pyrazol-4-yl]quinoline-2,4-dicarboxamide

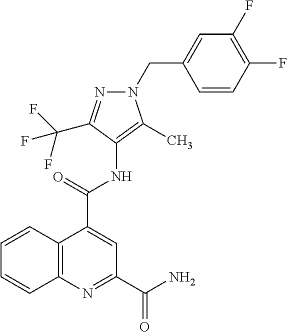 [SMILES: Cc1c(NC(=O)c2cc(C(N)=O)nc3ccccc23)c(C(F)(F)F)nn1Cc1ccc(F)c(F)c1]

In analogy to example 118), 113 mg (0.39 mmol) 1-(3,4-difluorobenzyl)-5-methyl-3-(trifluoromethyl)-1H-pyrazol-4-amine (intermediate 23C) and 70 mg (0.32 mmol) 2-carbamoylquinoline-4-carboxylic acid (intermediate 4A) were reacted to give after purification via preparative HPLC (method 4) 50 mg (29%) of the desired title compound.

¹H-NMR (400 MHz, DMSO d₆) δ (ppm)=2.32 (s, 3H), 5.48 (s, 2H), 7.15 (tt, 1H), 7.27-7.37 (m, 2H), 7.77-7.86 (m, 1H), 7.90 (s, 1H), 7.91-7.97 (m, 1H), 8.20 (t, 2H), 8.26 (s, 1H), 8.38 (s, 1H), 10.38 (s, 1H).

### Example 129

### 6-bromo-N-[1-(3,4-difluorobenzyl)-3,5-dimethyl-1H-pyrazol-4-yl]-2-(propan-2-yl)quinoline-4-carboxamide

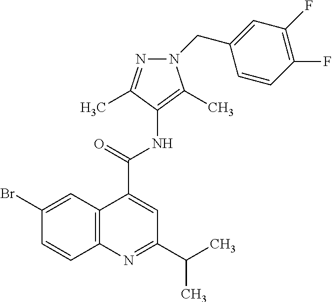 [SMILES: Cc1nn(Cc2ccc(F)c(F)c2)c(C)c1NC(=O)c1cc(C(C)C)nc2ccc(Br)cc12]

In analogy to example 118), 96.8 mg (0.41 mmol) 1-(3,4-difluorobenzyl)-3,5-dimethyl-1H-pyrazol-4-amine (intermediate 4C) and 100 mg (0.34 mmol) 6-bromo-2-isopropylquinoline-4-carboxylic acid were reacted to give after purification via preparative HPLC (method 3) 124 mg (64%) of the desired title compound.

¹H-NMR (400 MHz, DMSO d₆) δ (ppm)=1.36 (d, 6H), 2.14 (s, 3H), 2.17 (s, 3H), 3.28 (q, 1H), 5.25 (s, 2H), 7.01 (ddd, 1H), 7.17-7.25 (m, 1H), 7.43 (dt, 1H), 7.74 (s, 1H), 7.90 (dd, 1H), 7.97 (d, 1H), 8.32 (d, 1H), 9.96 (s, 1H).

### Example 130

### N⁴-[1-(4-fluorobenzyl)-5-methyl-3-(trifluoromethyl)-1H-pyrazol-4-yl]quinoline-2,4-dicarboxamide

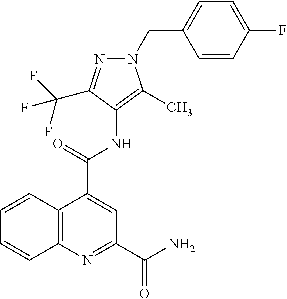 [SMILES: Cc1c(NC(=O)c2cc(C(N)=O)nc3ccccc23)c(C(F)(F)F)nn1Cc1ccc(F)cc1]

In analogy to example 118), 114 mg (0.42 mmol) 1-(4-fluorobenzyl)-5-methyl-3-(trifluoromethyl)-1H-pyrazol-4-amine (intermediate 25C) and 75 mg (0.35 mmol) 2-carbamoylquinoline-4-carboxylic acid (intermediate 4A) were reacted to give after purification via preparative HPLC (method 4) 50 mg (27%) of the desired title compound.

¹H-NMR (300 MHz, DMSO d₆) δ (ppm)=2.28 (s, 3H), 5.46 (s, 2H), 7.19-7.37 (m, 4H), 7.77-7.86 (m, 1H), 7.88-7.99 (m, 2H), 8.15-8.24 (m, 2H), 8.25 (s, 1H), 8.40 (s, 1H), 10.39 (s, 1H).

### Example 131

### methyl 4-{[4-({[6-bromo-2-(trifluoromethyl)quinolin-4-yl]carbonyl}amino)-3,5-dimethyl-1H-pyrazol-1-yl]methyl}benzoate

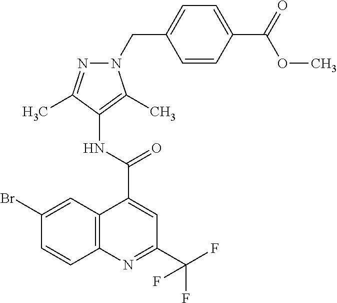 [SMILES: COC(=O)c1ccc(Cn2nc(C)c(NC(=O)c3cc(C(F)(F)F)nc4ccc(Br)cc34)c2C)cc1]

In analogy to example 118), 1.00 g (3.86 mmol) methyl 4-[(4-amino-3,5-dimethyl-1H-pyrazol-1-yl)methyl]benzoate (intermediate 31C) and 1.03 g (3.21 mmol) 6-bromo-2-(trifluoromethyl)quinoline-4-carboxylic acid (intermediate 1A) were reacted to give after stirring a reaction mixture which was diluted with water and ethyl acetate. A formed solid was isolated by filtration and were reacted to give 870 mg (45%) of the desired title compound. The filtrate was evaporated and purified via a Biotage chromatography system (25 g snap KP-Sil column, hexane/0 100% ethyl acetate, then ethyl acetate/0-60% methanol) and were reacted to give additional 660 mg (33%) of the desired title compound.

¹H-NMR (300 MHz, DMSO d₆) δ (ppm)=2.15 (s, 3H), 2.17 (s, 3H), 3.84 (s, 3H), 5.36 (s, 2H), 7.29 (d, 2H), 7.95 (d, 2H), 8.14 (dd, 1H), 8.22 (d, 1H), 8.29 (s, 1H), 8.50 (d, 1H), 10.19 (s, 1H).

### Example 132

### 4-{[4-({[6-bromo-2-(trifluoromethyl)quinolin-4-yl]carbonyl}amino)-3,5-dimethyl-1H-pyrazol-1-yl]methyl}benzoic acid

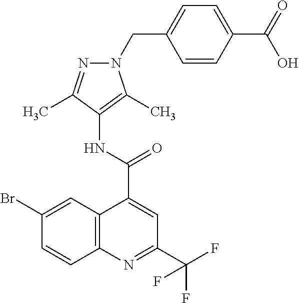 [SMILES: Cc1nn(Cc2ccc(C(=O)O)cc2)c(C)c1NC(=O)c1cc(C(F)(F)F)nc2ccc(Br)cc12]

To a solution of 720 mg (1.28 mmol) methyl 4-{[4-({[6-bromo-2-(trifluoromethyl)quinolin-4-yl]carbonyl}amino)-3,5-dimethyl-1H-pyrazol-1-yl]methyl}benzoate (example 131) in 11 mL methanol and 1 mL THF an aq. solution of 950 mg (23.7 mmol) sodium hydroxide in 21.8 mL water was added. This mixture was heated at 40° C. for 3 hours and evaporated after cooling to 25° C. To the residue was added 10 mL water and then aq. 10% sulfuric acid up to pH 3. The resulting solid was isolated by filtration and dried yielding 620 mg (84%) of the desired title compound.

¹H-NMR (300 MHz, DMSO d₆) δ (ppm)=2.15 (s, 3H), 2.17 (s, 3H), 5.35 (s, 2H), 7.22-7.32 (m, 2H), 7.89-7.97 (m, 2H), 8.14 (dd, 1H), 8.22 (d, 1H), 8.29 (s, 1H), 8.50 (d, 1H), 10.19 (s, 1H), 11.28 (s, 1H).

### Example 133

### 6-bromo-N-[1-(4-carbamoylbenzyl)-3,5-dimethyl-1H-pyrazol-4-yl]-2-(trifluoromethyl)quinoline-4-carboxamide

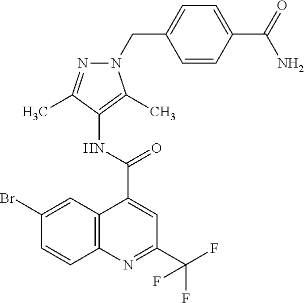 [SMILES: Cc1nn(Cc2ccc(C(N)=O)cc2)c(C)c1NC(=O)c1cc(C(F)(F)F)nc2ccc(Br)cc12]

To a solution of 200 mg (0.36 mmol) of the acid from example 132) in 1.32 mL DMSO was added 146 mg (0.38 mmol) HATU, 74 μL N,N-diisopropylethylamine and 0.61 mL 0.5M solution of ammonia in diaxane. The reaction mixture was stirred for 1 hour at 25° C. This mixture was directly purified via preparative HPLC (method 3) to obtain 43 mg (29%) of the desired title compound.

¹H-NMR (400 MHz, DMSO d₆) δ (ppm)=2.15 (s, 3H), 2.18 (s, 3H), 5.31 (s, 2H), 7.21 (d, 2H), 7.33 (br. s., 1H), 7.84 (d, 2H), 7.91 (br. s., 1H), 8.14 (dd, 1H), 8.22 (d, 1H), 8.28 (s, 1H), 8.50 (d, 1H), 10.17 (s, 1H).

### Example 134

### 6-bromo-N-{3,5-dimethyl-1-[4-(phenylcarbamoyl)benzyl]-1H-pyrazol-4-yl}-2-(trifluoromethyl)quinoline-4-carboxamide

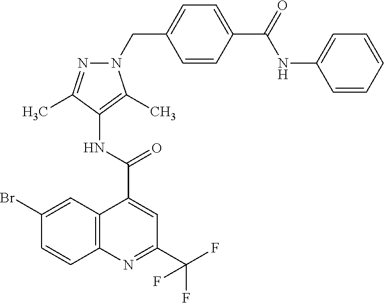 [SMILES: Cc1nn(Cc2ccc(C(=O)Nc3ccccc3)cc2)c(C)c1NC(=O)c1cc(C(F)(F)F)nc2ccc(Br)cc12]

In analogy to example 133), 88 mg (0.16 mmol) of the acid from example 132) and 18 mg (0.19 mmol) aniline were reacted to give after purification via preparative HPLC (method 3) 59 mg (56%) of the desired title compound.

¹H-NMR (300 MHz, DMSO ds) δ (ppm)=2.16 (s, 3H), 2.20 (s, 3H), 5.36 (s, 2H), 7.04-7.13 (m, 1H), 7.27-7.39 (m, 4H), 7.75 (d, 2H), 7.92 (d, 2H), 8.14 (dd, 1H), 8.23 (d, 1H), 8.30 (s, 1H), 8.50 (d, 1H), 10.19 (s, 1H), 10.21 (s, 1H).

### Example 135

### 6-bromo-N-{3,5-dimethyl-1-[4-(methylcarbamoyl)benzyl]-1H-pyrazol-4-yl}-2-(trifluoromethyl)quinoline-4-carboxamide

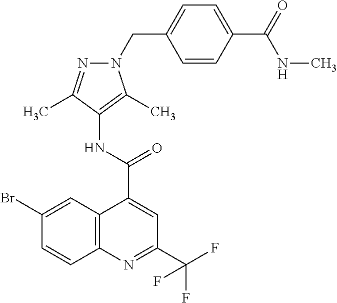 [SMILES: CNC(=O)c1ccc(Cn2nc(C)c(NC(=O)c3cc(C(F)(F)F)nc4ccc(Br)cc34)c2C)cc1]

In analogy to example 133), 88 mg (0.16 mmol) of the acid from example 132) and 96 μL (0.19 mmol) of a 2M solution of methylamine in THF were reacted to give after purification via preparative HPLC (method 3) 60 mg (63%) of the desired title compound.

¹H-NMR (300 MHz, DMSO d₆) δ (ppm)=2.15 (s, 3H), 2.18 (s, 3H), 2.76 (d, 3H), 5.31 (s, 2H), 7.22 (d, 2H), 7.79 (d, 2H), 8.14 (dd, 1H), 8.22 (d, 1H), 8.29 (s, 1H), 8.39 (q, 1H), 8.50 (d, 1H), 10.18 (s, 1H).

### Example 136

### 6-bromo-N-[3,5-dimethyl-1-(4-{[2-(morpholin-4-yl)ethyl]carbamoyl}benzyl)-1H-pyrazol-4-yl]-2-(trifluoromethyl)quinoline-4-carboxamide

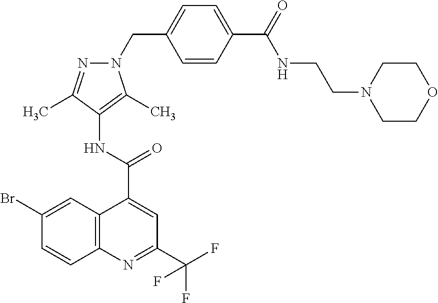 [SMILES: Cc1nn(Cc2ccc(C(=O)NCCN3CCOCC3)cc2)c(C)c1NC(=O)c1cc(C(F)(F)F)nc2ccc(Br)cc12]

In analogy to example 133), 150 mg (0.27 mmol) of the acid from example 132) and 30 mg (0.23 mmol) 2-(morpholin-4-yl)ethanamine were reacted to give after purification via preparative HPLC (method 3) 49 mg (63%) of the desired title compound.

¹H-NMR (300 MHz, DMSO d₆) δ (ppm)=2.15 (s, 3H), 2.18 (s, 3H), 3.03-3.24 (m, 2H), 3.46-3.72 (m, 6H), 4.00 (d, 2H), 5.33 (s, 2H), 7.29 (d, 2H), 7.84 (d, 2H), 8.15 (dd, 1H), 8.23 (d, 1H), 8.28 (s, 1H), 8.48 (d, 1H), 10.19 (s, 1H).

### Example 137

### 6-bromo-N-(1-{4-[(2-methoxyethyl)carbamoyl]benzyl}-3,5-dimethyl-1H-pyrazol-4-yl)-2-(trifluoromethyl)quinoline-4-carboxamide

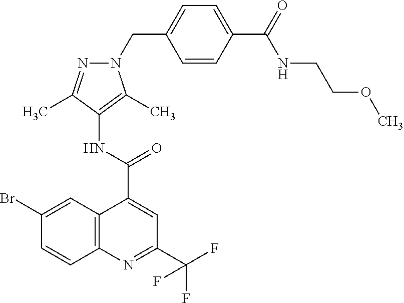 [SMILES: COCCNC(=O)c1ccc(Cn2nc(C)c(NC(=O)c3cc(C(F)(F)F)nc4ccc(Br)cc34)c2C)cc1]

In analogy to example 133), 88 mg (0.16 mmol) of the acid from example 132) and 15 mg (0.19 mmol) 2-methoxyethanamine were reacted to give after purification via preparative HPLC (method 3) 63 mg (61%) of the desired title compound.

¹H-NMR (300 MHz, DMSO d₆) δ (ppm)=2.15 (s, 3H), 2.18 (s, 3H), 3.25 (s, 3H), 3.37-3.48 (m, 4H), 5.31 (s, 2H), 7.19-7.27 (m, 2H), 7.77-7.85 (m, 2H), 8.14 (dd, 1H), 8.22 (d, 1H), 8.29 (s, 1H), 8.44-8.52 (m, 1H), 10.18 (s, 1H).

### Example 138

### 6-bromo-N-(3,5-dimethyl-1-{4-[(pyridin-3-ylmethyl)carbamoyl]benzyl}-1H-pyrazol-4-yl)-2-(trifluoromethyl)quinoline-4-carboxamide

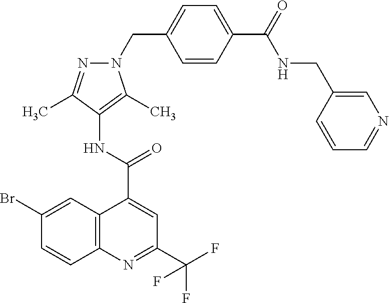 [SMILES: Cc1nn(Cc2ccc(C(=O)NCc3cccnc3)cc2)c(C)c1NC(=O)c1cc(C(F)(F)F)nc2ccc(Br)cc12]

In analogy to example 133), 150 mg (0.27 mmol) of the acid from example 132) and 25 mg (0.23 mmol) 1-(pyridin-3-yl)methanamine were reacted to give after purification via preparative HPLC (method 3) 47 mg (36%) of the desired title compound.

¹H-NMR (300 MHz, DMSO d₆) δ (ppm)=2.15 (s, 3H), 2.18 (s, 3H), 4.51 (d, 2H), 5.32 (s, 2H), 7.25 (d, 2H), 7.44 (dd, 1H), 7.77-7.89 (m, 3H), 8.14 (dd, 1H), 8.22 (d, 1H), 8.29 (s, 1H), 8.49 (d, 2H), 8.58 (s, 1H), 9.08 (t, 1H), 10.18 (s, 1H).

### Example 139

### 6-bromo-N-(1-{4-[(2-hydroxyethyl)carbamoyl]benzyl}-3,5-dimethyl-1H-pyrazol-4-yl)-2-(trifluoromethyl)quinoline-4-carboxamide

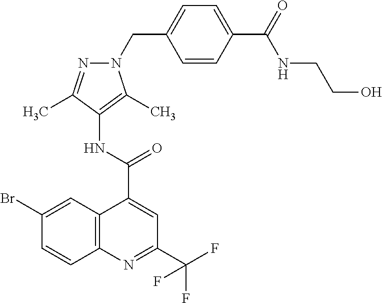 [SMILES: Cc1nn(Cc2ccc(C(=O)NCCO)cc2)c(C)c1NC(=O)c1cc(C(F)(F)F)nc2ccc(Br)cc12]

In analogy to example 133), 88 mg (0.16 mmol) of the acid from example 132) and 12 mg (0.19 mmol) 2-amino-ethanol were reacted to give after purification via preparative HPLC (method 3) 61 mg (61%) of the desired title compound.

¹H-NMR (400 MHz, DMSO d₆) δ (ppm)=2.15 (s, 3H), 2.17 (s, 3H), 3.45-3.53 (m, 2H), 4.72 (br. s., 1H), 5.31 (s, 2H), 7.20-7.25 (m, 2H), 7.79-7.85 (m, 2H), 8.11-8.16 (m, 1H), 8.22 (d, 1H), 8.28 (s, 1H), 8.40 (t, 1H), 8.47-8.51 (m, 1H), 10.20 (s, 1H).

### Example 140

### N-{1-[4-(benzylcarbamoyl)benzyl]-3,5-dimethyl-1H-pyrazol-4-yl}-6-bromo-2-(trifluoromethyl)quinoline-4-carboxamide

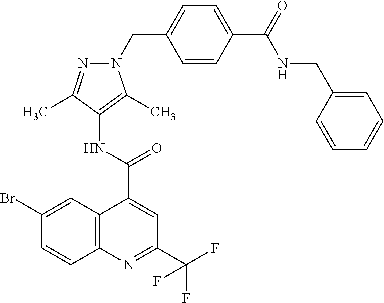 [SMILES: Cc1nn(Cc2ccc(C(=O)NCc3ccccc3)cc2)c(C)c1NC(=O)c1cc(C(F)(F)F)nc2ccc(Br)cc12]

In analogy to example 133), 88 mg (0.16 mmol) of the acid from example 132) and 21 mg (0.19 mmol) benzylamine were reacted to give after purification via preparative HPLC (method 3) 58 mg (54%) of the desired title compound.

¹H-NMR (300 MHz, DMSO d₆) δ (ppm)=2.15 (s, 3H), 2.18 (s, 3H), 4.47 (d, 2H), 5.32 (s, 2H), 7.19-7.36 (m, 7H), 7.87 (d, 2H), 8.14 (dd, 1H), 8.22 (d, 1H), 8.29 (s, 1H), 8.50 (d, 1H), 9.01 (t, 1H), 10.18 (s, 1H).

### Example 141

### 6-bromo-N-{3,5-dimethyl-1-[4-(morpholin-4-ylcarbonyl)benzyl]-1H-pyrazol-4-yl}-2-(trifluoromethyl)quinoline-4-carboxamide

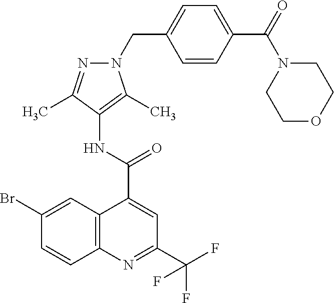 [SMILES: Cc1nn(Cc2ccc(C(=O)N3CCOCC3)cc2)c(C)c1NC(=O)c1cc(C(F)(F)F)nc2ccc(Br)cc12]

In analogy to example 133), 150 mg (0.27 mmol) of the acid from example 132) and 20 mg (0.23 mmol) morpholine were reacted to give after purification via preparative HPLC (method 3) 54 mg (44%) of the desired title compound.

¹H-NMR (300 MHz, DMSO d₆) δ (ppm)=2.15 (s, 3H), 2.19 (s, 3H), 3.36-3.69 (m, 8H), 5.30 (s, 2H), 7.19-7.25 (m, 2H), 7.37-7.44 (m, 2H), 8.14 (dd, 1H), 8.23 (d, 1H), 8.29 (s, 1H), 8.50 (d, 1H), 10.18 (s, 1H).

### Example 142

### 6-bromo-N-[1-(4-{[2-(dimethylamino)ethyl]carbamoyl}benzyl)-3,5-dimethyl-1H-pyrazol-4-yl]-2-(trifluoromethyl)quinoline-4-carboxamide

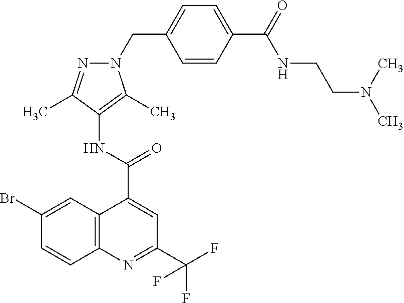 [SMILES: Cc1nn(Cc2ccc(C(=O)NCCN(C)C)cc2)c(C)c1NC(=O)c1cc(C(F)(F)F)nc2ccc(Br)cc12]

In analogy to example 133), 88 mg (0.16 mmol) of the acid from example 132) and 17 mg (0.19 mmol) N,N-dimethylethane-1,2-diamine were reacted to give after purification via preparative HPLC (method 3) 58 mg (56%) of the desired title compound.

¹H-NMR (300 MHz, DMSO d₆) δ (ppm)=2.15 (s, 3H), 2.18 (s, 3H), 2.82 (s, 6H), 3.22 (t, 2H), 3.58 (q, 2H), 5.33 (s, 2H), 7.24-7.32 (m, 2H), 7.79-7.86 (m, 2H), 8.14 (dd, 1H), 8.23 (d, 1H), 8.28 (s, 1H), 8.48 (d, 1H), 8.63 (t, 1H), 10.18 (s, 1H).

### Example 143

### 6-bromo-N-{1-[4-(dimethylcarbamoyl)benzyl]-3,5-dimethyl-1H-pyrazol-4-yl}-2-(trifluoromethyl)quinoline-4-carboxamide

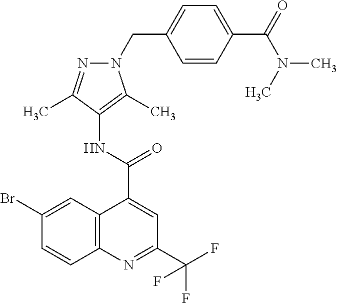 [SMILES: Cc1nn(Cc2ccc(C(=O)N(C)C)cc2)c(C)c1NC(=O)c1cc(C(F)(F)F)nc2ccc(Br)cc12]

In analogy to example 133), 88 mg (0.16 mmol) of the acid from example 132) and 96 μL (0.19 mmol) of a 2M solution of dimethylamine in tetrahydrofuran were reacted to give after purification via preparative HPLC (method 3) 64 mg (65%) of the desired title compound.

¹H-NMR (300 MHz, DMSO d₆) δ (ppm)=2.15 (s, 3H), 2.19 (s, 3H), 2.89 (s, 3H), 2.96 (s, 3H), 5.30 (s, 2H), 7.17-7.23 (m, 2H), 7.35-7.42 (m, 2H), 8.14 (dd, 1H), 8.22 (d, 1H), 8.29 (s, 1H), 8.50 (d, 1H), 10.18 (s, 1H).

### Example 144

### 6-bromo-2-cyclobutyl-N-[1-(3,4-difluorobenzyl)-3,5-dimethyl-1H-pyrazol-4-yl]quinoline-4-carboxamide

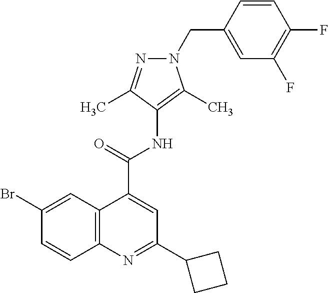 [SMILES: Cc1nn(Cc2ccc(F)c(F)c2)c(C)c1NC(=O)c1cc(C2CCC2)nc2ccc(Br)cc12]

In analogy to example 118), 93 mg (0.39 mmol) 1-(3,4-difluorobenzyl)-3,5-dimethyl-1H-pyrazol-4-amine (intermediate 4C) and 100 mg (0.33 mmol) 6-bromo-2-cyclobutylquinoline-4-carboxylic acid (intermediate 30A) were reacted to give after purification via preparative HPLC (method 3) 53 mg (28%) of the desired title compound.

¹H-NMR (400 MHz, DMSO d₆) δ (ppm)=1.87-1.97 (m, 1H), 2.03-2.12 (m, 1H), 2.13 (s, 3H), 2.17 (s, 3H), 2.29-2.47 (m, 4H), 3.90 (q, 1H), 5.25 (s, 2H), 7.01 (ddd, 1H), 7.21 (ddd, 1H), 7.43 (dt, 1H), 7.69 (s, 1H), 7.91 (dd, 1H), 7.99 (d, 1H), 8.32 (d, 1H), 9.96 (s, 1H).

### Example 145

### 6-chloro-2-cyclopropyl-N-[1-(3,4-difluorobenzyl)-3,5-dimethyl-1H-pyrazol-4-yl]quinoline-4-carboxamide

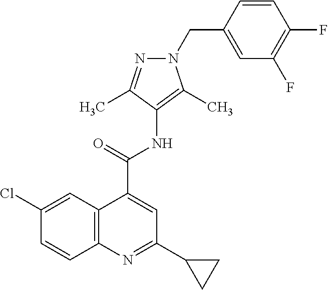 [SMILES: Cc1nn(Cc2ccc(F)c(F)c2)c(C)c1NC(=O)c1cc(C2CC2)nc2ccc(Cl)cc12]

In analogy to example 118), 86 mg (0.36 mmol) 1-(3,4-difluorobenzyl)-3,5-dimethyl-1H-pyrazol-4-amine (intermediate 4C) and 75 mg (0.30 mmol) 6-chloro-2-cyclopropylquinoline-4-carboxylic acid (intermediate 24A) were reacted to give after purification via preparative HPLC (method 3) 66 mg (42%) of the desired title compound.

¹H-NMR (400 MHz, DMSO d) δ (ppm)=1.06-1.20 (m, 4H), 2.13 (s, 3H), 2.17 (s, 3H), 2.36-2.43 (m, 1H), 5.25 (s, 2H), 6.97-7.05 (m, 1H), 7.22 (ddd, 1H), 7.43 (dt, 1H), 7.71-7.78 (m, 2H), 7.93 (d, 1H), 8.12 (d, 1H), 9.98 (s, 1H).

### Example 146

### 6-bromo-2-cyclobutyl-N-[1-(4-fluorobenzyl)-3,5-dimethyl-1H-pyrazol-4-yl]quinoline-4-carboxamide

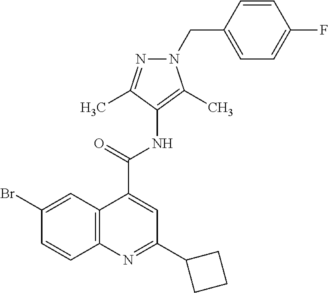 [SMILES: Cc1nn(Cc2ccc(F)cc2)c(C)c1NC(=O)c1cc(C2CCC2)nc2ccc(Br)cc12]

In analogy to example 118), 67 mg (0.31 mmol) 1-(4-fluorobenzyl)-3,5-dimethyl-1H-pyrazol-4-amine (intermediate 1C) and 85 mg (0.28 mmol) 6-bromo-2-cyclobutylquinoline-4-carboxylic acid (intermediate 30A) were reacted to give after purification via preparative HPLC (method 3) 82 mg (53%) of the desired title compound.

¹H-NMR (400 MHz, DMSO d₆) δ (ppm)=1.84-1.95 (m, 1H), 2.03-2.11 (m, 1H), 2.11-2.14 (m, 3H), 2.16 (s, 3H), 2.30-2.47 (m, 4H), 3.90 (q, 1H), 5.24 (s, 2H), 7.14-7.27 (m, 4H), 7.68 (s, 1H), 7.90 (dd, 1H), 7.99 (d, 1H), 8.32 (d, 1H), 9.95 (s, 1H).

### Example 147

### 6-bromo-N-{1-[(6-cyanopyridin-3-yl)methyl]-3,5-dimethyl-1H-pyrazol-4-yl}-2-(trifluoromethyl)quinoline-4-carboxamide

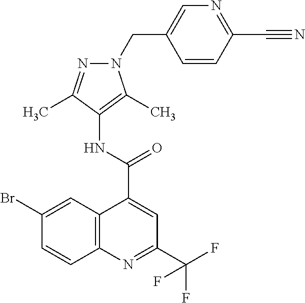 [SMILES: Cc1nn(Cc2ccc(C#N)nc2)c(C)c1NC(=O)c1cc(C(F)(F)F)nc2ccc(Br)cc12]

In analogy to example 118), 100 mg (0.38 mmol) 5-[(4-amino-3,5-dimethyl-1H-pyrazol-1-yl)methyl]pyridine-2-carbonitrile (intermediate 29C) and 100 mg (0.31 mmol) 6-bromo-2-(trifluoromethyl)quinoline-4-carboxylic acid (intermediate 1A) were reacted to give after purification via preparative HPLC (method 4) 8 mg (4.6%) of the desired title compound.

¹H-NMR (400 MHz, DMSO d₆) δ (ppm)=2.14 (s, 3H), 2.23 (s, 3H), 5.43 (s, 2H), 7.75 (dd, 1H), 8.05 (d, 1H), 8.14 (dd, 1H), 8.22 (d, 1H), 8.29 (s, 1H), 8.50 (d, 1H), 8.60 (d, 1H), 10.19 (s, 1H).

### Example 148

### 6-bromo-N-[1-(4-chlorobenzyl)-3,5-dimethyl-1H-pyrazol-4-yl]-2-(trifluoromethyl)quinoline-4-carboxamide

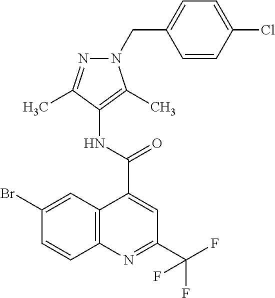 [SMILES: Cc1nn(Cc2ccc(Cl)cc2)c(C)c1NC(=O)c1cc(C(F)(F)F)nc2ccc(Br)cc12]

In analogy to example 118), 111 mg (0.47 mmol) 1-(4-chlorobenzyl)-3,5-dimethyl-1H-pyrazol-4-amine (intermediate 27C) and 100 mg (0.31 mmol) 6-bromo-2-(trifluoromethyl)quinoline-4-carboxylic acid (intermediate 1A) were reacted to give after purification via preparative HPLC (method 3) 121 mg (68%) of the desired title compound.

¹H-NMR (400 MHz, DMSO d₆) δ (ppm)=2.14 (s, 3H), 2.18 (s, 3H), 5.26 (s, 2H), 7.17-7.22 (m, 2H), 7.39-7.45 (m, 2H), 8.14 (dd, 1H), 8.22 (d, 1H), 8.28 (s, 1H), 8.49 (d, 1H), 10.16 (s, 1H).

### Example 149

### methyl 4-{[1-(4-fluorobenzyl)-3,5-dimethyl-1H-pyrazol-4-yl]carbamoyl}-2-(trifluoromethyl)quinoline-6-carboxylate

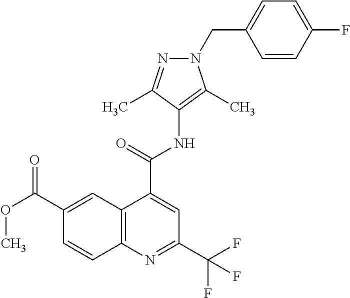 [SMILES: COC(=O)c1ccc2nc(C(F)(F)F)cc(C(=O)Nc3c(C)nn(Cc4ccc(F)cc4)c3C)c2c1]

A solution of 150 mg (0.29 mmol) 6-bromo-N-[1-(4-fluorobenzyl)-3,5-dimethyl-1H-pyrazol-4-yl]-2-(trifluoromethyl)quinoline-4-carboxamide (example 9), 35 μL (0.86 mmol) methanol, 114 mg (0.43 mmol) Mo(CO)₆ (Molybdenum hexacarbonyl), 8.35 (0.029 mmol) Tri-tert.-butylphosphonium tetrafluoroborate, 27.0 mg (0.029 mmol) trans-Di(mu-acetato)bis o-(di-o-tolylphosphino)benzyl dipalladium (II), and 131 mg (0.86 mmol) 1,8-Diazabicclo[5.4.0]undec-7-en in 2.0 mL THF was heated at 125° C. for 20 minutes in a microwave reactor. This mixture together with a second one resulting from a second experiment starting with 500 mg (0.96 mmol) 6-bromo-N-[1-(4-fluorobenzyl)-3,5-dimethyl-1H-pyrazol-4-yl]-2-(trifluoromethyl)quinoline-4-carboxamide (example 9) were absorbed on Isolute and purified via a Biotage chromatography system (10 g snap KP-Sil column, hexane/10-70% ethyl acetate) were reacted to give a 306 mg of crude product. This crude product was purified via two successive HPLC runs yielding 21 mg (4.2%) of the desired title compound.

¹H-NMR (300 MHz, DMSO d₆) δ (ppm)=2.17 (s, 3H), 2.21 (s, 3H), 3.94 (s, 3H), 5.25 (s, 2H), 7.13-7.29 (m, 4H), 8.34 (s, 1H), 8.35-8.44 (m, 2H), 8.97 (s, 1H), 10.19 (s, 1H).

### Example 150

### methyl (4-{[4-({[6-bromo-2-(trifluoromethyl)quinolin-4-yl]carbonyl}amino)-3,5-dimethyl-1H-pyrazol-1-yl]methyl}phenyl)acetate

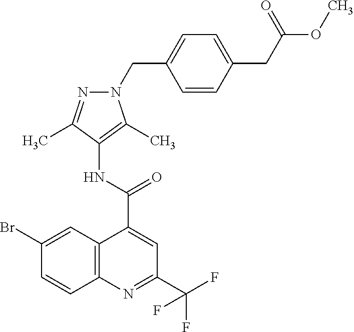 [SMILES: COC(=O)Cc1ccc(Cn2nc(C)c(NC(=O)c3cc(C(F)(F)F)nc4ccc(Br)cc34)c2C)cc1]

In analogy to example 118), 1.00 g (3.66 mmol) methyl {4-[(4-amino-3,5-dimethyl-1H-pyrazol-1-yl)methyl]phenyl}acetate (intermediate 32C) and 0.98 g (3.05 mmol) 6-bromo-2-(trifluoromethyl)quinoline-4-carboxylic acid (intermediate 1A) were reacted to give after stirring a reaction mixture which was diluted with water and ethyl acetate. After phase separation the aqueous phase was extracted with ethyl acetate. Then the combined organic phases were washed with brine, dried over sodium sulfate, filtered and evaporated to dryness. The crude product was purified via a Biotage chromatography system (25 g snap KP-Sil column, hexane/50-100% ethyl acetate, then ethyl acetate/0-90% methanol) to give 960 mg (50%) of the desired title compound.

¹H-NMR (400 MHz, DMSO d₆) δ (ppm)=2.14 (s, 3H), 2.18 (s, 3H), 3.59 (s, 3H), 3.65 (s, 2H), 5.23 (s, 2H), 7.13 (d, 2H), 7.24 (d, 2H), 8.14 (dd, 1H), 8.22 (d, 1H), 8.27 (s, 1H), 8.50 (d, 1H), 10.14 (s, 1H).

### Example 151

### (4-{[4-({[6-bromo-2-(trifluoromethyl)quinolin-4-yl]carbonyl}amino)-3,5-dimethyl-1H-pyrazol-1-yl]methyl}phenyl)acetic acid

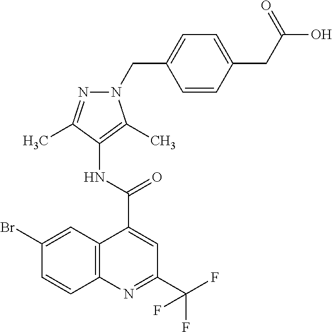 [SMILES: Cc1nn(Cc2ccc(CC(=O)O)cc2)c(C)c1NC(=O)c1cc(C(F)(F)F)nc2ccc(Br)cc12]

In analogy to example 132), 960 mg (1.67 mmol) methyl (4-{[4-({[6-bromo-2-(trifluoromethyl)quinolin-4-yl]carbonyl}amino)-3,5-dimethyl-1H-pyrazol-1-yl]methyl}phenyl)acetate (example 150) were reacted to give 1.02 g (103%, purity 95%) of the desired title compound.

¹H-NMR (300 MHz, DMSO d₆) δ (ppm)=2.14 (s, 3H), 2.18 (s, 3H), 3.54 (s, 2H), 5.23 (s, 2H), 7.08-7.15 (m, 2H), 7.20-7.27 (m, 2H), 8.14 (dd, 1H), 8.22 (d, 1H), 8.28 (s, 1H), 8.50 (d, 1H), 10.16 (s, 1H), 11.83 (br. s., 1H).

### Example 152

### N-{1-[4-(2-amino-2-oxoethyl)benzyl]-3,5-dimethyl-1H-pyrazol-4-yl}-6-bromo-2-(trifluoromethyl)quinoline-4-carboxamide

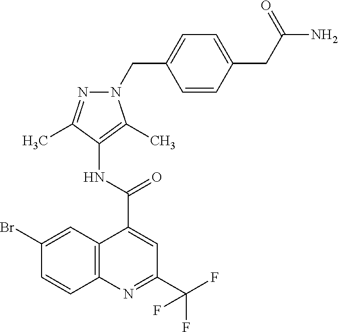 [SMILES: Cc1nn(Cc2ccc(CC(N)=O)cc2)c(C)c1NC(=O)c1cc(C(F)(F)F)nc2ccc(Br)cc12]

In analogy to example 133), 100 mg (0.18 mmol) (4-{[4-({[6-bromo-2-(trifluoromethyl)quinolin-4-yl]carbonyl}amino)-3,5-dimethyl-1H-pyrazol-1-yl]methyl}phenyl)acetic acid (example 151) were reacted to give after purification via preparative HPLC (method 3) 44 mg (43%) of the desired title compound.

¹H-NMR (400 MHz, DMSO d₆) δ (ppm)=2.14 (s, 3H), 2.18 (s, 3H), 3.34 (s, 2H), 5.21 (s, 2H), 6.83 (br. s., 1H), 7.10 (d, 2H), 7.22 (d, 2H), 7.41 (br. s., 1H), 8.14 (dd, 1H), 8.22 (d, 1H), 8.27 (s, 1H), 8.50 (d, 1H), 10.13 (s, 1H).

### Example 153

### 6-bromo-N-(3,5-dimethyl-1-{4-[2-(methylamino)-2-oxoethyl]benzyl}-1H-pyrazol-4-yl)-2-(trifluoromethyl)quinoline-4-carboxamide

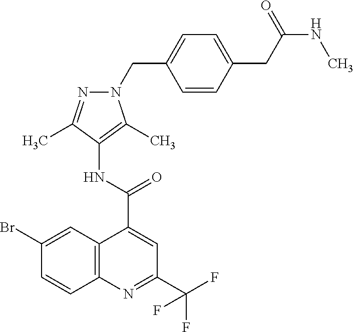 [SMILES: CNC(=O)Cc1ccc(Cn2nc(C)c(NC(=O)c3cc(C(F)(F)F)nc4ccc(Br)cc34)c2C)cc1]

In analogy to example 135), 100 mg (0.18 mmol) (4-{[4-({[6-bromo-2-(trifluoromethyl)quinolin-4-yl]carbonyl}amino)-3,5-dimethyl-1H-pyrazol-1-yl]methyl}phenyl)acetic acid (example 151) were reacted to give after purification via preparative HPLC (method 3) 42 mg (40%) of the desired title compound.

¹H-NMR (400 MHz, DMSO d₆) δ (ppm)=2.14 (s, 3H), 2.18 (s, 3H), 2.55 (d, 3H), 3.35 (s, 2H), 5.21 (s, 2H), 7.07-7.13 (m, 2H), 7.18-7.25 (m, 2H), 7.90 (q, 1H), 8.14 (dd, 1H), 8.22 (d, 1H), 8.27 (s, 1H), 8.49 (d, 1H), 10.13 (s, 1H).

### Example 154

### N-(1-{4-[2-(benzylamino)-2-oxoethyl]benzyl}-3,5-dimethyl-1H-pyrazol-4-yl)-6-bromo-2-(trifluoromethyl)quinoline-4-carboxamide

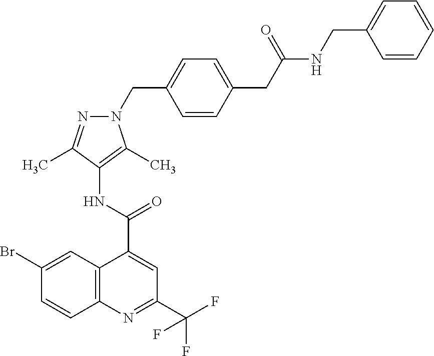 [SMILES: Cc1nn(Cc2ccc(CC(=O)NCc3ccccc3)cc2)c(C)c1NC(=O)c1cc(C(F)(F)F)nc2ccc(Br)cc12]

In analogy to example 140), 100 mg (0.18 mmol) (4-{[4-({[6-bromo-2-(trifluoromethyl)quinolin-4-yl]carbonyl}amino)-3,5-dimethyl-1H-pyrazol-1-yl]methyl}phenyl)acetic acid (example 151) were reacted to give after purification via preparative HPLC (method 3) 15 mg (12%) of the desired title compound.

¹H-NMR (300 MHz, DMSO d₆) δ (ppm)=2.14 (s, 3H), 2.18 (s, 3H), 3.45 (s, 2H), 4.24 (d, 2H), 5.22 (s, 2H), 7.11 (d, 2H), 7.17-7.33 (m, 7H), 8.14 (dd, 1H), 8.22 (d, 1H), 8.28 (s, 1H), 8.48-8.56 (m, 2H), 10.15 (s, 1H).

### Example 155

### 6-bromo-N-(3,5-dimethyl-1-{4-[2-oxo-2-(phenylamino)ethyl]benzyl}-1H-pyrazol-4-yl)-2-(trifluoromethyl)quinoline-4-carboxamide

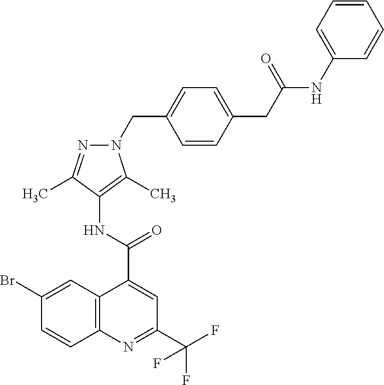 [SMILES: Cc1nn(Cc2ccc(CC(=O)Nc3ccccc3)cc2)c(C)c1NC(=O)c1cc(C(F)(F)F)nc2ccc(Br)cc12]

In analogy to example 134), 100 mg (0.18 mmol) (4-{[4-({[6-bromo-2-(trifluoromethyl)quinolin-4-yl]carbonyl}amino)-3,5-dimethyl-1H-pyrazol-1-yl]methyl}phenyl)acetic acid (example 151) were reacted to give after purification via preparative HPLC (method 3) 51 mg (44%) of the desired title compound.

¹H-NMR (300 MHz, DMSO d₆) δ (ppm)=2.14 (s, 3H), 2.18 (s, 3H), 3.61 (s, 2H), 5.22 (s, 2H), 6.97-7.06 (m, 1H), 7.14 (d, 2H), 7.22-7.33 (m, 4H), 7.57 (d, 2H), 8.14 (dd, 1H), 8.22 (d, 1H), 8.28 (s, 1H), 8.49 (d, 1H), 10.10-10.18 (m, 2H).

### Example 156

### 6-bromo-N-(1-{4-[2-(dimethylamino)-2-oxoethyl]benzyl}-3,5-dimethyl-1H-pyrazol-4-yl)-2-(trifluoromethyl)quinoline-4-carboxamide

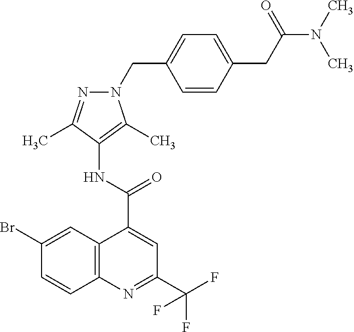 [SMILES: Cc1nn(Cc2ccc(CC(=O)N(C)C)cc2)c(C)c1NC(=O)c1cc(C(F)(F)F)nc2ccc(Br)cc12]

In analogy to example 143), 100 mg (0.18 mmol) (4-{[4-({[6-bromo-2-(trifluoromethyl)quinolin-4-yl]carbonyl}amino)-3,5-dimethyl-1H-pyrazol-1-yl]methyl}phenyl)acetic acid (example 151) were reacted to give after purification via preparative HPLC (method 3) 57 mg (53%) of the desired title compound.

¹H-NMR (400 MHz, DMSO d₆) δ (ppm)=2.14 (s, 3H), 2.19 (s, 3H), 2.81 (s, 3H), 2.98 (s, 3H), 3.65 (s, 2H), 5.22 (s, 2H), 7.11 (d, 2H), 7.19 (d, 2H), 8.14 (dd, 1H), 8.22 (d, 1H), 8.28 (s, 1H), 8.50 (d, 1H), 10.14 (s, 1H).

### Example 157

### 6-bromo-N-[3,5-dimethyl-1-(4-{2-oxo-2-[(pyridin-3-ylmethyl)amino]ethyl}benzyl)-1H-pyrazol-4-yl]-2-(trifluoromethyl)quinoline-4-carboxamide

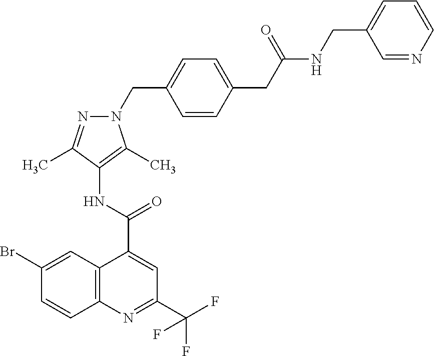 [SMILES: Cc1nn(Cc2ccc(CC(=O)NCc3cccnc3)cc2)c(C)c1NC(=O)c1cc(C(F)(F)F)nc2ccc(Br)cc12]

In analogy to example 138), 100 mg (0.18 mmol) (4-{[4-({[6-bromo-2-(trifluoromethyl)quinolin-4-yl]carbonyl}amino)-3,5-dimethyl-1H-pyrazol-1-yl]methyl}phenyl)acetic acid (example 151) were reacted to give after purification via preparative HPLC (method 3) 44 mg (37%) of the desired title compound.

¹H-NMR (300 MHz, DMSO d₆) δ (ppm)=2.14 (s, 3H), 2.18 (s, 3H), 3.48 (s, 2H), 4.35 (d, 2H), 5.22 (s, 2H), 7.09-7.16 (m, 2H), 7.20-7.27 (m, 2H), 7.64 (dd, 1H), 7.97 (d, 1H), 8.14 (dd, 1H), 8.22 (d, 1H), 8.27 (s, 1H), 8.49 (d, 1H), 8.57-8.67 (m, 3H), 10.14 (s, 1H).

### Example 158

### 6-bromo-N-(3,5-dimethyl-1-{4-[2-(morpholin-4-yl)-2-oxoethyl]benzyl}-1H-pyrazol-4-yl)-2-(trifluoromethyl)quinoline-4-carboxamide

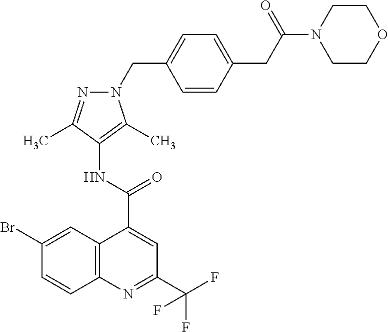 [SMILES: Cc1nn(Cc2ccc(CC(=O)N3CCOCC3)cc2)c(C)c1NC(=O)c1cc(C(F)(F)F)nc2ccc(Br)cc12]

In analogy to example 141), 100 mg (0.18 mmol) (4-{[4-({[6-bromo-2-(trifluoromethyl)quinolin-4-yl]carbonyl}amino)-3,5-dimethyl-1H-pyrazol-1-yl]methyl}phenyl)acetic acid (example 151) were reacted to give after purification via preparative HPLC (method 3) 57 mg (49%) of the desired title compound.

¹H-NMR (300 MHz, DMSO d₆) δ (ppm)=2.14 (s, 3H), 2.18 (s, 3H), 3.38-3.57 (m, 8H), 3.68 (s, 2H), 5.22 (s, 2H), 7.07-7.15 (m, 2H), 7.16-7.23 (m, 2H), 8.14 (dd, 1H), 8.22 (d, 1H), 8.27 (s, 1H), 8.50 (d, 1H), 10.14 (s, 1H).

### Example 159

### 6-bromo-N-[1-(4-{2-[(2-hydroxyethyl)amino]-2-oxoethyl}benzyl)-3,5-dimethyl-1H-pyrazol-4-yl]-2-(trifluoromethyl)quinoline-4-carboxamide

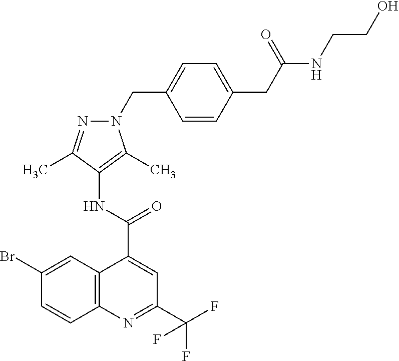 [SMILES: Cc1nn(Cc2ccc(CC(=O)NCCO)cc2)c(C)c1NC(=O)c1cc(C(F)(F)F)nc2ccc(Br)cc12]

In analogy to example 139), 100 mg (0.18 mmol) (4-{[4-({[6-bromo-2-(trifluoromethyl)quinolin-4-yl]carbonyl}amino)-3,5-dimethyl-1H-pyrazol-1-yl]methyl}phenyl)acetic acid (example 151) were reacted to give after purification via preparative HPLC (method 3) 48 mg (44%) of the desired title compound.

¹H-NMR (300 MHz, DMSO d₆) δ (ppm)=2.14 (s, 3H), 2.18 (s, 3H), 3.09 (q, 2H), 3.33-3.43 (m, 4H), 4.66 (t, 1H), 5.21 (s, 2H), 7.06-7.13 (m, 2H), 7.19-7.26 (m, 2H), 8.03 (t, 1H), 8.14 (dd, 1H), 8.22 (d, 1H), 8.28 (s, 1H), 8.49 (d, 1H), 10.15 (s, 1H).

### Example 160

### 6-bromo-N-[3,5-dimethyl-1-(pyridin-4-ylmethyl)-1H-pyrazol-4-yl]-2-(trifluoromethyl)quinoline-4-carboxamide

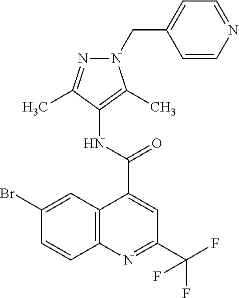 [SMILES: Cc1nn(Cc2ccncc2)c(C)c1NC(=O)c1cc(C(F)(F)F)nc2ccc(Br)cc12]

In analogy to example 118), 94.8 mg (0.47 mmol) 3,5-dimethyl-1-(pyridin-4-ylmethyl)-1H-pyrazol-4-amine (intermediate 16C) and 100 mg (0.31 mmol) 6-bromo-2-(trifluoromethyl)quinoline-4-carboxylic acid (intermediate 1A) were reacted to give after purification via preparative HPLC (method 3) 80 mg (47%) of the desired title compound.

¹H-NMR (400 MHz, DMSO d₆) δ (ppm)=2.16 (s, 3H), 2.19 (s, 3H), 5.46 (s, 2H), 7.32 (d, 2H), 8.15 (dd, 1H), 8.23 (d, 1H), 8.30 (s, 1H), 8.50 (d, 1H), 8.69 (d, 2H), 10.22 (s, 1H).

### Example 161

### 6-bromo-N-[1-(4-cyanobenzyl)-5-methyl-3-(trifluoromethyl)-1H-pyrazol-4-yl]-2-(trifluoromethyl)quinoline-4-carboxamide

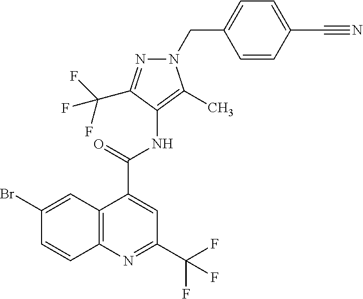 [SMILES: Cc1c(NC(=O)c2cc(C(F)(F)F)nc3ccc(Br)cc23)c(C(F)(F)F)nn1Cc1ccc(C#N)cc1]

In analogy to example 118), 84.1 mg (0.30 mmol) 4-{[4-amino-5-methyl-3-(trifluoromethyl)-1H-pyrazol-1-yl]methyl}benzonitrile (intermediate 26C) and 90 mg (0.25 mmol) 6-bromo-2-(trifluoromethyl)quinoline-4-carboxylic acid (intermediate 1A) were reacted to give after purification via preparative HPLC (method 4) 38 mg (25%) of the desired title compound.

¹H-NMR (400 MHz, DMSO d₆) δ (ppm)=2.29 (s, 3H), 5.60 (s, 2H), 7.35-7.40 (m, 2H), 7.86-7.92 (m, 2H), 8.15 (dd, 1H), 8.20-8.27 (m, 2H), 8.42 (d, 1H), 10.52 (s, 1H).

### Example 162

### 2-cyclopropyl-N-[1-(3,4-difluorobenzyl)-3,5-dimethyl-1H-pyrazol-4-yl]-6-fluoroquinoline-4-carboxamide

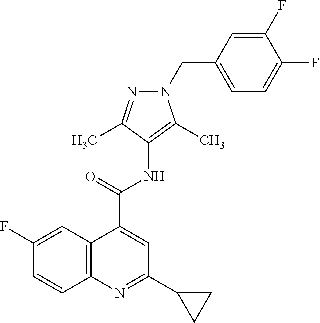 [SMILES: Cc1nn(Cc2ccc(F)c(F)c2)c(C)c1NC(=O)c1cc(C2CC2)nc2ccc(F)cc12]

In analogy to example 118), 84.1 mg (0.30 mmol) 1-(3,4-difluorobenzyl)-3,5-dimethyl-1H-pyrazol-4-amine (intermediate 4C) and 90 mg (0.25 mmol) 2-cyclopropyl-6-fluoroquinoline-4-carboxylic acid (intermediate 26A) were reacted to give after purification via preparative HPLC (method 3) 142 mg (66%) of the desired title compound.

¹H-NMR (300 MHz, DMSO d₆) δ (ppm)=1.05-1.19 (m, 4H), 2.13 (s, 3H), 2.17 (s, 3H), 2.35-2.42 (m, 1H), 5.25 (s, 2H), 6.96-7.05 (m, 1H), 7.17-7.27 (m, 1H), 7.43 (dt, 1H), 7.66 (td, 1H), 7.72 (s, 1H), 7.80 (dd, 1H), 7.98 (dd, 1H), 9.95 (s, 1H).

### Example 163

### 2-(dimethylamino)-N-[1-(4-fluorobenzyl)-3,5-dimethyl-1H-pyrazol-4-yl]quinoline-4-carboxamide

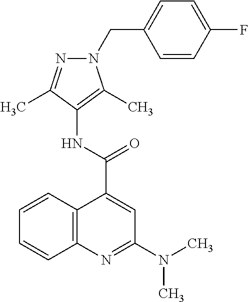 [SMILES: Cc1nn(Cc2ccc(F)cc2)c(C)c1NC(=O)c1cc(N(C)C)nc2ccccc12]

In analogy to example 118), 84.1 mg (0.30 mmol) 1-(4-fluorobenzyl)-3,5-dimethyl-1H-pyrazol-4-amine (intermediate 1C) and 90 mg (0.25 mmol) 2-(dimethylamino)quinoline-4-carboxylic acid were reacted to give after purification via preparative HPLC (method 4) 90 mg (44%) of the desired title compound.

¹H-NMR (300 MHz, DMSO d₆) δ (ppm)=2.12 (s, 3H), 2.16 (s, 3H), 3.20 (s, 6H), 5.23 (s, 2H), 7.13-7.27 (m, 6H), 7.48-7.64 (m, 2H), 7.87 (d, 1H), 9.78 (s, 1H).

### Example 164

### 6-bromo-N-[1-(4-hydroxybenzyl)-3,5-dimethyl-1H-pyrazol-4-yl]-2-(trifluoromethyl)quinoline-4-carboxamide

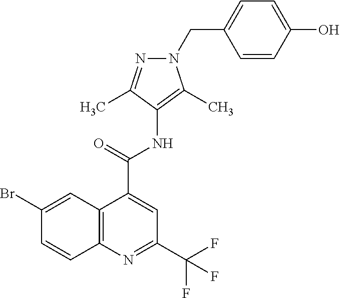 [SMILES: Cc1nn(Cc2ccc(O)cc2)c(C)c1NC(=O)c1cc(C(F)(F)F)nc2ccc(Br)cc12]

To a solution of 100 mg (0.19 mmol) 6-bromo-N-[1-(4-methoxybenzyl)-3,5-dimethyl-1H-pyrazol-4-yl]-2-(trifluoromethyl)quinoline-4-carboxamide (example 3) was added dropwise at 25° C. 6.8 mL of a 1M solution of BBr₃ in dichloromethane. After stirring for 20 hours the mixture was cooled with ice and 1 mL methanol was carefully added to the mixture. Afterwards, 8.2 mL conc. aq. sodium bicarbonate was added and stirring was continued for 30 minutes. After the addition of 50 mL water the mixture was extracted three times with 80 mL ethyl acetate. The combined organic phases were washed with brine, dried over sodium sulfate, filtered and evaporated. The crude product was purified via HPLC (method 4) yielding 45 mg (44%) of the desired title compound.

¹H-NMR (400 MHz, DMSO d₆) δ (ppm)=2.13 (s, 3H), 2.17 (s, 3H), 5.10 (s, 2H), 6.68-6.75 (m, 2H), 7.00-7.06 (m, 2H), 8.14 (dd, 1H), 8.22 (d, 1H), 8.26 (s, 1H), 8.49 (d, 1H), 9.37 (s, 1H), 10.12 (s, 1H).

### Example 165

### 6-chloro-N-[1-(3,4-difluorobenzyl)-3,5-dimethyl-1H-pyrazol-4-yl]-2-(trifluoromethyl)quinoline-4-carboxamide

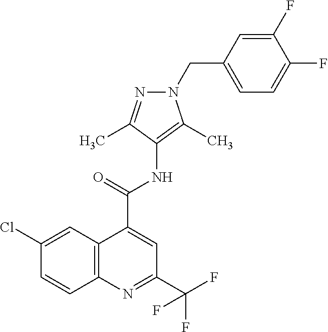 [SMILES: Cc1nn(Cc2ccc(F)c(F)c2)c(C)c1NC(=O)c1cc(C(F)(F)F)nc2ccc(Cl)cc12]

In analogy to example 118), 103 mg (0.44 mmol) 1-(3,4-difluorobenzyl)-3,5-dimethyl-1H-pyrazol-4-amine (intermediate 4C) and 100 mg (0.36 mmol) 6-chloro-2-(trifluoromethyl)quinoline-4-carboxylic acid (intermediate 12A) were reacted to give after purification via preparative HPLC (method 4) 89 mg (47%) of the desired title compound.

¹H-NMR (300 MHz, DMSO d₆) δ (ppm)=2.14 (s, 3H), 2.19 (s, 3H), 5.25 (s, 2H), 7.01 (ddd, 1H), 7.22 (ddd, 1H), 7.43 (dt, 1H), 8.04 (dd, 1H), 8.28-8.35 (m, 3H), 10.18 (s, 1H).

### Example 166

### N-[1-(4-fluorobenzyl)-3,5-dimethyl-1H-pyrazol-4-yl]-6-phenyl-2-(trifluoromethyl)quinoline-4-carboxamide

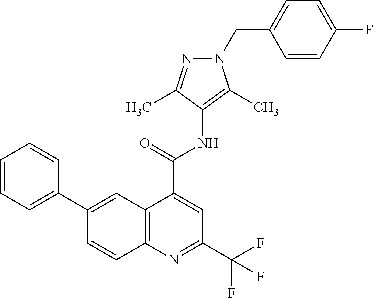 [SMILES: Cc1nn(Cc2ccc(F)cc2)c(C)c1NC(=O)c1cc(C(F)(F)F)nc2ccc(-c3ccccc3)cc12]

A mixture of 150 mg (0.29 mmol) 6-bromo-N-[1-(4-fluorobenzyl)-3,5-dimethyl-1H-pyrazol-4-yl]-2-(trifluoromethyl)quinoline-4-carboxamide (example 9), 70 mg (0.58 mmol) phenylboronic acid, 210 mg (0.29 mmol) [1,1′-bis (diphenylphosphino)ferrocene]dichloropalladium(II), 91.5 mg (0.86 mmol) sodium carbonate in a 0.38 mL water and 2.95 mL dioxan was heated for 90 minutes at 105° C. in a microwave reactor. After cooling the mixture was purified via a Biotage chromatography system (10 g snap KP-Sil column, hexane/50-100% ethyl acetate) yielding 72 mg (46%) of the desired title compound.

¹H-NMR (400 MHz, DMSO d₆) δ (ppm)=2.15 (s, 3H), 2.19 (s, 3H), 5.24 (s, 2H), 7.14-7.27 (m, 4H), 7.44-7.51 (m, 1H), 7.52-7.59 (m, 2H), 7.77-7.82 (m, 2H), 8.22 (s, 1H), 8.31-8.38 (m, 2H), 8.50 (s, 1H), 10.14 (s, 1H).

### Example 167

### 6,8-dichloro-N-[1-(4-cyanobenzyl)-5-methyl-3-(trifluoromethyl)-1H-pyrazol-4-yl]-2-(trifluoromethyl)quinoline-4-carboxamide

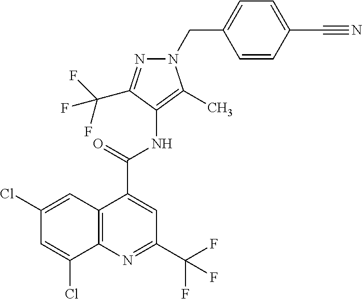 [SMILES: Cc1c(NC(=O)c2cc(C(F)(F)F)nc3c(Cl)cc(Cl)cc23)c(C(F)(F)F)nn1Cc1ccc(C#N)cc1]

In analogy to example 118), 87 mg (0.31 mmol) 4-{[4-amino-5-methyl-3-(trifluoromethyl)-1H-pyrazol-1-yl]methyl}benzonitrile (intermediate 26C) and 80 mg (0.26 mmol) 6,8-dichloro-2-(trifluoromethyl)quinoline-4-carboxylic acid (intermediate 11A) were reacted to give after purification via preparative HPLC (method 4) 68 mg (44%) of the desired title compound.

¹H-NMR (300 MHz, DMSO d) δ (ppm)=2.29 (s, 3H), 5.60 (s, 2H), 7.33-7.40 (m, 2H), 7.85-7.92 (m, 2H), 8.19 (d, 1H), 8.38 (s, 1H), 8.42 (d, 1H), 10.58 (s, 1H).

### Example 168

### N-[1-(3,4-difluorobenzyl)-3,5-dimethyl-1H-pyrazol-4-yl]-7-fluoro-2-(trifluoromethyl)quinoline-4-carboxamide

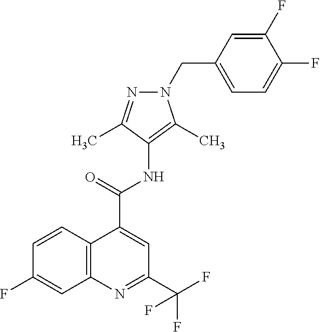 [SMILES: Cc1nn(Cc2ccc(F)c(F)c2)c(C)c1NC(=O)c1cc(C(F)(F)F)nc2cc(F)ccc12]

In analogy to example 118), 82 mg (0.35 mmol) 1-(3,4-difluorobenzyl)-3,5-dimethyl-1H-pyrazol-4-amine (intermediate 4C) and 75 mg (0.29 mmol) 7-fluoro-2-(trifluoromethyl)quinoline-4-carboxylic acid (intermediate 17A) were reacted to give after purification via preparative HPLC (method 3) 88 mg (57%) of the desired title compound.

¹H-NMR (400 MHz, DMSO d₆) δ (ppm)=2.15 (s, 3H), 2.19 (s, 3H), 5.25 (s, 2H), 6.97-7.04 (m, 1H), 7.16-7.24 (m, 1H), 7.43 (dt, 1H), 7.86 (td, 1H), 8.08 (dd, 1H), 8.20 (s, 1H), 8.37 (dd, 1H), 10.13 (s, 1H).

### Example 169

### N-[1-(4-cyanobenzyl)-3,5-dimethyl-1H-pyrazol-4-yl]-6,7-difluoro-2-(trifluoromethyl)quinoline-4-carboxamide

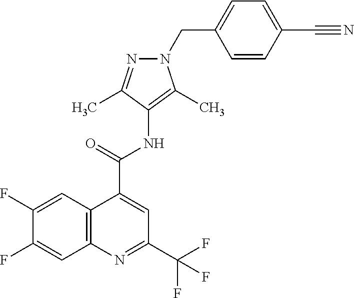 [SMILES: Cc1nn(Cc2ccc(C#N)cc2)c(C)c1NC(=O)c1cc(C(F)(F)F)nc2cc(F)c(F)cc12]

In analogy to example 118), 98 mg (0.43 mmol) 4-[(4-amino-3,5-dimethyl-1H-pyrazol-1-yl)methyl]benzonitrile (intermediate 8C) and 100 mg (0.36 mmol) 6,7-difluoro-2-(trifluoromethyl)quinoline-4-carboxylic acid (intermediate 19A) were reacted to give after purification via preparative HPLC (method 3) 121 mg (66%) of the desired title compound.

¹H-NMR (300 MHz, DMSO d₆) δ (ppm)=2.14 (s, 3H), 2.17 (s, 3H), 5.38 (s, 2H), 7.23-7.33 (m, 2H), 7.80-7.88 (m, 2H), 8.26 (dd, 1H), 8.31 (s, 1H), 8.41 (dd, 1H), 10.21 (s, 1H).

### Example 170

### 6-bromo-N-[1-(cyclohexylmethyl)-3,5-dimethyl-1H-pyrazol-4-yl]-2-(trifluoromethyl)quinoline-4-carboxamide

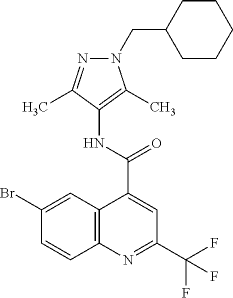 [SMILES: Cc1nn(CC2CCCCC2)c(C)c1NC(=O)c1cc(C(F)(F)F)nc2ccc(Br)cc12]

In analogy to example 118), 77 mg (0.31 mmol) 1-(cyclohexylmethyl)-3,5-dimethyl-1H-pyrazol-4-amine (intermediate 33C) and 100 mg (0.31 mmol) 6-bromo-2-(trifluoromethyl)quinoline-4-carboxylic acid (intermediate 1A) were reacted to give after purification via preparative HPLC (method 3) 87 mg (52%) of the desired title compound.

¹H-NMR (300 MHz, DMSO d₆) δ (ppm)=0.97 (q, 2H), 1.06-1.28 (m, 3H), 1.49-1.87 (m, 6H), 2.11 (s, 3H), 2.19 (s, 3H), 3.80 (d, 2H), 8.14 (dd, 1H), 8.22 (d, 1H), 8.28 (s, 1H), 8.49 (d, 1H), 10.12 (s, 1H).

### Example 171

### 6-bromo-N-[3,5-dimethyl-1-(pyridin-3-ylmethyl)-1H-pyrazol-4-yl]-2-(trifluoromethyl)quinoline-4-carboxamide

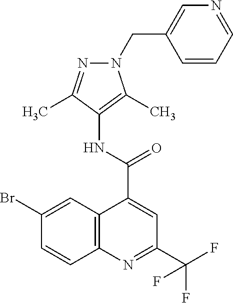 [SMILES: Cc1nn(Cc2cccnc2)c(C)c1NC(=O)c1cc(C(F)(F)F)nc2ccc(Br)cc12]

In analogy to example 118), 90 mg (0.45 mmol) 3,5-dimethyl-1-(pyridin-2-ylmethyl)-1H-pyrazol-4-amine (intermediate 18C) and 119 mg (0.37 mmol) 6-bromo-2-(trifluoromethyl)quinoline-4-carboxylic acid (intermediate 1A) were reacted to give after purification via preparative HPLC (method 3) 74 mg (38%) of the desired title compound.

¹H-NMR (300 MHz, DMSO d₆) δ (ppm)=2.14 (s, 3H), 2.23 (s, 3H), 5.32 (s, 2H), 7.43 (dd, 1H), 7.62 (d, 1H), 8.14 (dd, 1H), 8.22 (d, 1H), 8.29 (s, 1H), 8.45-8.56 (m, 3H), 10.18 (s, 1H).

### Example 172

### N⁴-[1-(4-fluorobenzyl)-3,5-dimethyl-1H-pyrazol-4-yl]-N²-methylquinoline-2,4-dicarboxamide

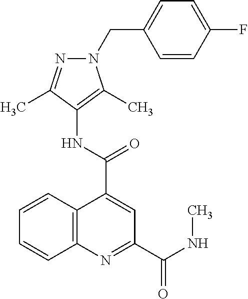 [SMILES: CNC(=O)c1cc(C(=O)Nc2c(C)nn(Cc3ccc(F)cc3)c2C)c2ccccc2n1]

In analogy to example 118), 79 mg (0.36 mmol) 1-(4-fluorobenzyl)-3,5-dimethyl-1H-pyrazol-4-amine (intermediate 1C) and 69 mg (0.30 mmol) 2-(methylcarbamoyl)quinoline-4-carboxylic acid (intermediate 6A) were reacted to give after purification via preparative HPLC (method 3) 98 mg (70%) of the desired title compound.

¹H-NMR (300 MHz, DMSO d₆) δ (ppm)=2.14 (s, 3H), 2.18 (s, 3H), 2.91 (d, 3H), 5.24 (s, 2H), 7.13-7.29 (m, 4H), 7.76-7.85 (m, 1H), 7.94 (td, 1H), 8.16-8.28 (m, 3H), 9.00 (q, 1H), 10.06 (s, 1H).

### Example 173

### N-[1-(3,4-difluorobenzyl)-3,5-dimethyl-1H-pyrazol-4-yl]-5-fluoro-2-(trifluoromethyl)quinoline-4-carboxamide

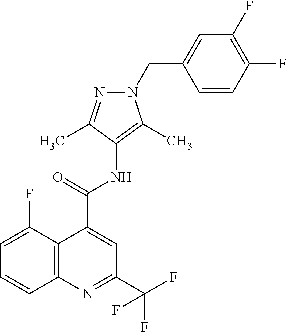 [SMILES: Cc1nn(Cc2ccc(F)c(F)c2)c(C)c1NC(=O)c1cc(C(F)(F)F)nc2cccc(F)c12]

In analogy to example 118), 110 mg (0.46 mmol) 1-(3,4-difluorobenzyl)-3,5-dimethyl-1H-pyrazol-4-amine (intermediate 4C) and 100 mg (0.39 mmol) 5-fluoro-2-(trifluoromethyl)quinoline-4-carboxylic acid (intermediate 16A) were reacted to give after purification via preparative HPLC a material, which was solved in 30 mL ethyl acetate. This organic phase was washed with water, conc. aq. sodium bicarbonate, brine, dried over sodium sulfate, filtered and evaporated to give 134 mg (69%) of the desired title compound.

¹H-NMR (300 MHz, DMSO d₆) δ (ppm)=2.15 (s, 3H), 2.19 (s, 3H), 5.25 (s, 2H), 6.96-7.05 (m, 1H), 7.21 (ddd, 1H), 7.43 (dt, 1H), 7.86 (td, 1H), 8.09 (dd, 1H), 8.20 (s, 1H), 8.37 (dd, 1H), 10.14 (s, 1H).

### Example 174

### N-[1-(3,4-difluorobenzyl)-3,5-dimethyl-1H-pyrazol-4-yl]-6-fluoro-2-(trifluoromethyl)quinoline-4-carboxamide

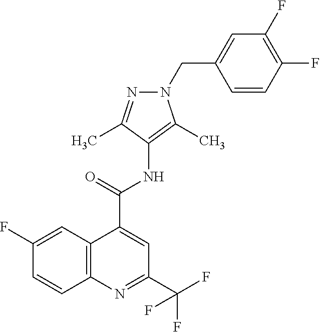 [SMILES: Cc1nn(Cc2ccc(F)c(F)c2)c(C)c1NC(=O)c1cc(C(F)(F)F)nc2ccc(F)cc12]

In analogy to example 118), 79 mg (0.36 mmol) 1-(3,4-difluorobenzyl)-3,5-dimethyl-1H-pyrazol-4-amine (intermediate 4C) and 69 mg (0.30 mmol) 6-fluoro-2-(trifluoromethyl)quinoline-4-carboxylic acid (intermediate 18A) were reacted to give after purification via preparative HPLC (method 3) 118 mg (57%) of the desired title compound.

¹H-NMR (400 MHz, DMSO d₆) δ (ppm)=2.14 (s, 3H), 2.19 (s, 3H), 5.25 (s, 2H), 6.97-7.05 (m, 1H), 7.17-7.26 (m, 1H), 7.43 (dt, 1H), 7.91-8.03 (m, 2H), 8.28 (s, 1H), 8.37 (dd, 1H), 10.14 (s, 1H).

### Example 175

### N-[1-(2-cyanobenzyl)-3,5-dimethyl-1H-pyrazol-4-yl]-6,7-difluoro-2-(trifluoromethyl)quinoline-4-carboxamide

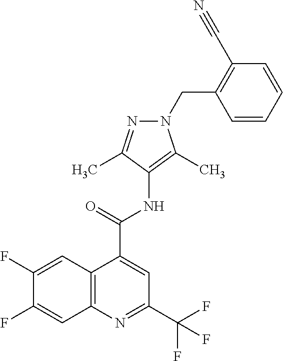 [SMILES: Cc1nn(Cc2ccccc2C#N)c(C)c1NC(=O)c1cc(C(F)(F)F)nc2cc(F)c(F)cc12]

In analogy to example 118), 74 mg (0.33 mmol) 2-[(4-amino-3,5-dimethyl-1H-pyrazol-1-yl)methyl]benzonitrile (intermediate 10C) and 75 mg (0.27 mmol) 6,7-difluoro-2-(trifluoromethyl)quinoline-4-carboxylic acid (intermediate 19A) were reacted to give after purification via preparative HPLC (method 4) 96 mg (70%) of the desired title compound.

¹H-NMR (400 MHz, DMSO d₆) δ (ppm)=2.12 (s, 3H), 2.25 (s, 3H), 5.43 (s, 2H), 7.09 (d, 1H), 7.52 (td, 1H), 7.67-7.73 (m, 1H), 7.89 (dd, 1H), 8.26 (dd, 1H), 8.31 (s, 1H), 8.40 (dd, 1H), 10.22 (s, 1H).

### Example 176

### 6-bromo-N-ethyl-N-[1-(4-fluorobenzyl)-3,5-dimethyl-1H-pyrazol-4-yl]-2-(trifluoromethyl)quinoline-4-carboxamide

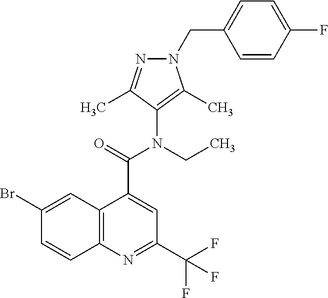 [SMILES: CCN(C(=O)c1cc(C(F)(F)F)nc2ccc(Br)cc12)c1c(C)nn(Cc2ccc(F)cc2)c1C]

To a solution of 200 mg (0.38 mmol) 6-bromo-N-[1-(4-fluorobenzyl)-3,5-dimethyl-1H-pyrazol-4-yl]-2-(trifluoromethyl)quinoline-4-carboxamide (example 9) in 2.0 mL N,N-dimethylformamide was added at 0° C. 16 mg (0.40, 60% in mineral oil). After stirring for 1 hour at 0° C. 37 μL ethyl iodide was added and stirring was continued for 3 hours at 25° C. This mixture was purified via HPLC (method 4) yielding 126 mg (56%) of the desired title compound.

¹H-NMR (300 MHz, DMSO ds) δ (ppm)=1.16 (t, 3H), 1.84 (s, 3H), 2.03 (s, 3H), 3.66 (dq, 1H), 4.04 (dq, 1H), 4.85-5.05 (m, 2H), 6.19 (dd, 2H), 6.86 (t, 2H), 7.95 (s, 1H), 8.04 (dd, 1H), 8.13 (d, 1H), 8.21 (d, 1H).

### Example 177

### N-[1-(3,4-difluorobenzyl)-3,5-dimethyl-1H-pyrazol-4-yl]-6,7-difluoro-2-(trifluoromethyl)quinoline-4-carboxamide

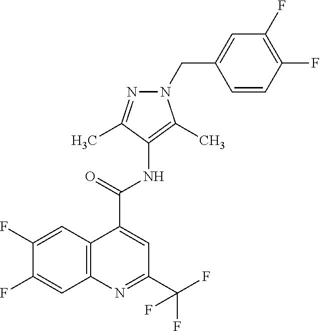 [SMILES: Cc1nn(Cc2ccc(F)c(F)c2)c(C)c1NC(=O)c1cc(C(F)(F)F)nc2cc(F)c(F)cc12]

In analogy to example 118), 103 mg (0.36 mmol) 1-(3,4-difluorobenzyl)-3,5-dimethyl-1H-pyrazol-4-amine (intermediate 4C) and 100 mg (0.43 mmol) 6,7-difluoro-2-(trifluoromethyl)quinoline-4-carboxylic acid (intermediate 19A) were reacted to give after purification via preparative HPLC (method 3) 85 mg (43%) of the desired title compound.

¹H-NMR (400 MHz, DMSO d₆) δ (ppm)=2.14 (s, 3H), 2.18-2.19 (m, 3H), 5.25 (s, 2H), 7.01 (ddd, 1H), 7.20 (ddd, 1H), 7.43 (dt, 1H), 8.25 (dd, 1H), 8.29 (s, 1H), 8.39 (dd, 1H), 10.18 (s, 1H).

### Example 178

### N⁴-[1-(4-fluorobenzyl)-5-methyl-3-(trifluoromethyl)-1H-pyrazol-4-yl]-N2-methylquinoline-2,4-dicarboxamide

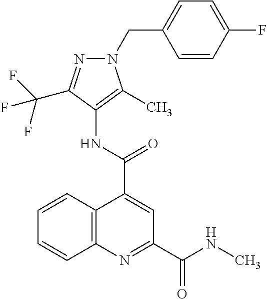 [SMILES: CNC(=O)c1cc(C(=O)Nc2c(C(F)(F)F)nn(Cc3ccc(F)cc3)c2C)c2ccccc2n1]

In analogy to example 118), 98 mg (0.36 mmol) 1-(4-fluorobenzyl)-5-methyl-3-(trifluoromethyl)-1H-pyrazol-4-amine (intermediate 25C) and 69 mg (0.30 mmol) 2-(methylcarbamoyl)quinoline-4-carboxylic acid (intermediate 6A) were reacted to give after purification via preparative HPLC (method 3) 75 mg (48%) of the desired title compound.

¹H-NMR (300 MHz, DMSO d₆) δ (ppm)=2.28 (s, 3H), 2.91 (d, 3H), 5.46 (s, 2H), 7.20-7.35 (m, 4H), 7.78-7.85 (m, 1H), 7.94 (ddd, 1H), 8.20 (dd, 2H), 8.24 (s, 1H), 9.01 (q, 1H), 10.40 (s, 1H).

### Example 179

### 6-bromo-N-[1-(2,4-difluorobenzyl)-5-methyl-3-(trifluoromethyl)-1H-pyrazol-4-yl]-2-(trifluoromethyl)quinoline-4-carboxamide

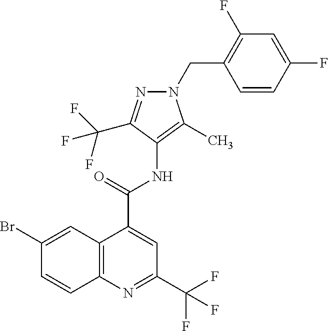 [SMILES: Cc1c(NC(=O)c2cc(C(F)(F)F)nc3ccc(Br)cc23)c(C(F)(F)F)nn1Cc1ccc(F)cc1F]

In analogy to example 118), 87 mg (0.30 mmol) 1-(2,4-difluorobenzyl)-5-methyl-3-(trifluoromethyl)-1H-pyrazol-4-amine (intermediate 24C) and 80 mg (0.25 mmol) 6-bromo-2-(trifluoromethyl)quinoline-4-carboxylic acid (intermediate 1A) were reacted to give after purification via preparative HPLC (method 4) 112 mg (74%) of the desired title compound.

¹H-NMR (400 MHz, DMSO d₆) δ (ppm)=2.34 (s, 3H), 5.48 (s, 2H), 7.15 (td, 1H), 7.27-7.37 (m, 2H), 8.15 (dd, 1H), 8.21-8.27 (m, 2H), 8.41 (d, 1H), 10.49 (s, 1H).

### Example 180

### 6-bromo-N-{1-[(5-chlorothiophen-2-yl)methyl]-3,5-dimethyl-1H-pyrazol-4-yl}-2-(trifluoromethyl)quinoline-4-carboxamide

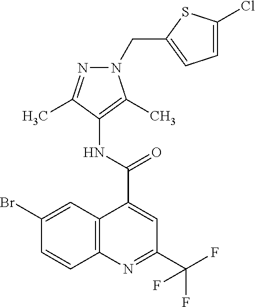 [SMILES: Cc1nn(Cc2ccc(Cl)s2)c(C)c1NC(=O)c1cc(C(F)(F)F)nc2ccc(Br)cc12]

In analogy to example 118), 90 mg (0.37 mmol) 1-[(5-chloro-2-thienyl)methyl]-3,5-dimethyl-1H-pyrazol-4-amine (intermediate 34C) and 99 mg (0.31 mmol) 6-bromo-2-(trifluoromethyl)quinoline-4-carboxylic acid (intermediate 1A) were reacted to give after purification via preparative HPLC (method 3) 19 mg (11%) of the desired title compound.

¹H-NMR (300 MHz, DMSO d₆) δ (ppm)=2.14 (s, 3H), 2.24 (s, 3H), 5.37 (s, 2H), 6.95-7.02 (m, 2H), 8.14 (dd, 1H), 8.22 (d, 1H), 8.28 (s, 1H), 8.48 (d, 1H), 10.16 (s, 1H).

### Example 181

### N-[1-(4-fluorobenzyl)-3,5-dimethyl-1H-pyrazol-4-yl]-2-(trifluoromethyl)quinoline-4-carboxamide

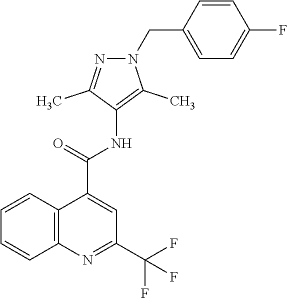 [SMILES: Cc1nn(Cc2ccc(F)cc2)c(C)c1NC(=O)c1cc(C(F)(F)F)nc2ccccc12]

In analogy to example 118), 81 mg (0.37 mmol) 1-(4-fluorobenzyl)-3,5-dimethyl-1H-pyrazol-4-amine (intermediate 1C) and 75 mg (0.31 mmol) 2-(trifluoromethyl)quinoline-4-carboxylic acid were reacted to give after purification via preparative HPLC (method 3) 84 mg (56%) of the desired title compound.

¹H-NMR (300 MHz, DMSO d₆) δ (ppm)=2.15 (s, 3H), 2.19 (s, 3H), 5.24 (s, 2H), 7.13-7.28 (m, 4H), 7.86-7.94 (m, 1H), 7.96-8.05 (m, 1H), 8.18 (s, 1H), 8.27 (d, 2H), 10.09 (s, 1H).

### Example 182

### N-[1-(4-cyanobenzyl)-5-methyl-3-(trifluoromethyl)-1H-pyrazol-4-yl]-6,7-difluoro-2-(trifluoromethyl)quinoline-4-carboxamide

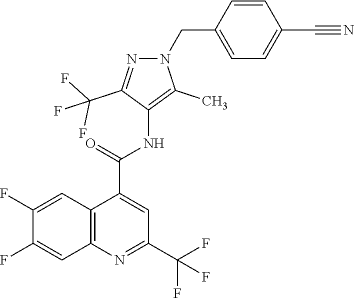 [SMILES: Cc1c(NC(=O)c2cc(C(F)(F)F)nc3cc(F)c(F)cc23)c(C(F)(F)F)nn1Cc1ccc(C#N)cc1]

In analogy to example 118), 97 mg (0.35 mmol) 4-{[4-amino-5-methyl-3-(trifluoromethyl)-1H-pyrazol-1-yl]methyl}benzonitrile (intermediate 26C) and 80 mg (0.29 mmol) 6,7-difluoro-2-(trifluoromethyl)quinoline-4-carboxylic acid (intermediate 19A) were reacted to give after purification via preparative HPLC (method 4) 102 mg (63%) of the desired title compound.

¹H-NMR (400 MHz, DMSO d₆) δ (ppm)=2.28 (s, 3H), 5.60 (s, 2H), 7.34-7.40 (m, 2H), 7.85-7.92 (m, 2H), 8.15 (dd, 1H), 8.25 (s, 1H), 8.42 (dd, 1H), 10.54 (s, 1H).

### Example 183

### N-{1-[(6-cyanopyridin-3-yl)methyl]-3,5-dimethyl-1H-pyrazol-4-yl}-6,7-difluoro-2-(trifluoromethyl)quinoline-4-carboxamide

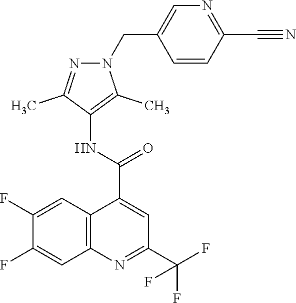 [SMILES: Cc1nn(Cc2ccc(C#N)nc2)c(C)c1NC(=O)c1cc(C(F)(F)F)nc2cc(F)c(F)cc12]

In analogy to example 118), 115 mg (0.43 mmol) 5-[(4-amino-3,5-dimethyl-1H-pyrazol-1-yl)methyl]pyridine-2-carbonitrile (intermediate 29C) and 100 mg (0.36 mmol) 6,7-difluoro-2-(trifluoromethyl)quinoline-4-carboxylic acid (intermediate 19A) were reacted to give after purification via preparative HPLC (method 4) 10 mg (5.1%) of the desired title compound.

¹H-NMR (600 MHz, DMSO d₆) δ (ppm)=2.13 (s, 3H), 2.22 (s, 3H), 5.43 (s, 2H), 7.75 (dd, 1H), 8.05 (d, 1H), 8.25 (dd, 1H), 8.30 (s, 1H), 8.40 (dd, 1H), 8.59 (d, 1H), 10.21 (s, 1H).

### Example 184

### 6-bromo-N-[1-(4-fluorobenzyl)-3,5-dimethyl-1H-pyrazol-4-yl]quinoline-4-carboxamide

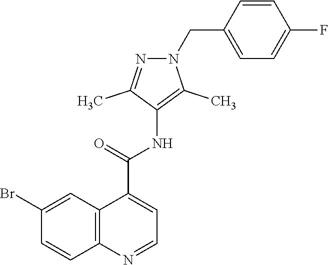 [SMILES: Cc1nn(Cc2ccc(F)cc2)c(C)c1NC(=O)c1ccnc2ccc(Br)cc12]

In analogy to example 118), 72 mg (0.33 mmol) 1-(4-fluorobenzyl)-3,5-dimethyl-1H-pyrazol-4-amine (intermediate 1C) and 75 mg (0.30 mmol) 6-bromoquinoline-4-carboxylic acid were reacted to give after purification via preparative HPLC (method 3) 73 mg (50%) of the desired title compound.

¹H-NMR (300 MHz, DMSO d₆) δ (ppm)=2.13 (s, 3H), 2.17 (s, 3H), 5.24 (s, 2H), 7.14-7.28 (m, 4H), 7.81 (d, 1H), 7.97 (dd, 1H), 8.07 (d, 1H), 8.39 (d, 1H), 9.07 (d, 1H), 10.00 (s, 1H).

### Example 185

### 6-bromo-N-[1-(3,4-difluorobenzyl)-3,5-dimethyl-1H-pyrazol-4-yl]-2-methylquinoline-4-carboxamide

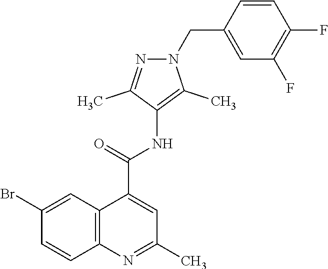 [SMILES: Cc1cc(C(=O)Nc2c(C)nn(Cc3ccc(F)c(F)c3)c2C)c2cc(Br)ccc2n1]

In analogy to example 118), 107 mg (0.45 mmol) 1-(3,4-difluorobenzyl)-3,5-dimethyl-1H-pyrazol-4-amine (intermediate 4C) and 100 mg (0.38 mmol) 6-bromo-2-methylquinoline-4-carboxylic acid were reacted to give after purification via preparative HPLC (method 3) 107 mg (53%) of the desired title compound.

¹H-NMR (300 MHz, DMSO d₆) δ (ppm)=2.13 (s, 3H), 2.17 (s, 3H), 2.71 (s, 3H), 5.25 (s, 2H), 7.01 (ddd, 1H), 7.23 (ddd, 1H), 7.43 (dt, 1H), 7.73 (s, 1H), 7.87-7.93 (m, 1H), 7.93-7.98 (m, 1H), 8.31 (d, 1H), 9.98 (s, 1H).

### Example 186

### N-[1-(3-chloro-4-fluorobenzyl)-3,5-dimethyl-1H-pyrazol-4-yl]-2,6-dimethylquinoline-4-carboxamide

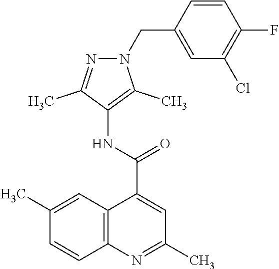 [SMILES: Cc1ccc2nc(C)cc(C(=O)Nc3c(C)nn(Cc4ccc(F)c(Cl)c4)c3C)c2c1]

In analogy to example 118), 151 mg (0.60 mmol) 1-(3-chloro-4-fluorobenzyl)-3,5-dimethyl-1H-pyrazol-4-amine (intermediate 28C) and 100 mg (0.50 mmol) 2,6-dimethylquinoline-4-carboxylic acid were reacted to give after purification via preparative HPLC (method 3) 168 mg (74%) of the desired title compound.

¹H-NMR (400 MHz, DMSO d₆) δ (ppm)=2.14 (s, 3H), 2.18 (s, 3H), 2.48 (s, 3H), 2.70 (s, 3H), 5.25 (s, 2H), 7.19 (ddd, 1H), 7.38-7.45 (m, 2H), 7.59-7.65 (m, 2H), 7.86-7.92 (m, 2H), 9.87 (s, 1H).

### Example 187

### 6-bromo-N-{3,5-dimethyl-1-[(1-methyl-1H-pyrazol-3-yl)methyl]-1H-pyrazol-4-yl}-2-(trifluoromethyl)quinoline-4-carboxamide

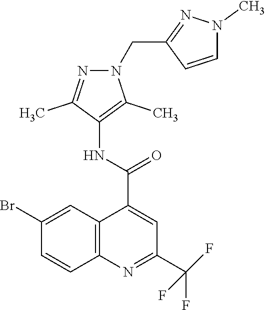 [SMILES: Cc1nn(Cc2ccn(C)n2)c(C)c1NC(=O)c1cc(C(F)(F)F)nc2ccc(Br)cc12]

In analogy to example 118), 210 mg (1.02 mmol) 3,5-dimethyl-1-[(1-methyl-1H-pyrazol-3-yl)methyl]-1H-pyrazol-4-amine (intermediate 35C) and 273 mg (0.85 mmol) 6-bromo-2-(trifluoromethyl)quinoline-4-carboxylic acid (intermediate 1A) were reacted to give after purification via preparative HPLC (method 3) 36 mg (8.1%) of the desired title compound.

¹H-NMR (400 MHz, DMSO d₆) δ (ppm)=2.11 (s, 3H), 2.24 (s, 3H), 3.79 (s, 3H), 5.12 (s, 2H), 6.07 (d, 1H), 7.60 (d, 1H), 8.14 (dd, 1H), 8.22 (d, 1H), 8.27 (s, 1H), 8.48 (d, 1H), 10.11 (s, 1H).

### Example 188

### N-[1-(4-cyanobenzyl)-5-methyl-3-(trifluoromethyl)-1H-pyrazol-4-yl]-2,6-dimethylquinoline-4-carboxamide

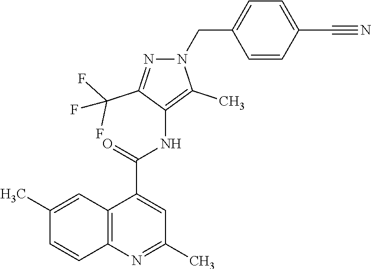 [SMILES: Cc1ccc2nc(C)cc(C(=O)Nc3c(C(F)(F)F)nn(Cc4ccc(C#N)cc4)c3C)c2c1]

In analogy to example 118), 134 mg (0.48 mmol) 4-{[4-amino-5-methyl-3-(trifluoromethyl)-1H-pyrazol-1-yl]methyl}benzonitrile (intermediate 26C) and 80 mg (0.40 mmol) 2,6-dimethylquinoline-4-carboxylic acid were reacted to give after purification via preparative HPLC (method 4) 132 mg (70%) of the desired title compound.

¹H-NMR (300 MHz, DMSO d₆) δ (ppm)=2.25 (s, 3H), 2.47 (s, 3H), 2.69 (s, 3H), 5.59 (s, 2H), 7.38 (d, 2H), 7.52 (s, 1H), 7.62 (dd, 1H), 7.81 (s, 1H), 7.86-7.93 (m, 3H), 10.22 (s, 1H).

### Example 189

### 7-bromo-N-[1-(3,4-difluorobenzyl)-3,5-dimethyl-1H-pyrazol-4-yl]-2-(trifluoromethyl)quinoline-4-carboxamide

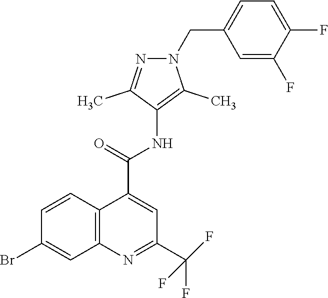 [SMILES: Cc1nn(Cc2ccc(F)c(F)c2)c(C)c1NC(=O)c1cc(C(F)(F)F)nc2cc(Br)ccc12]

In analogy to example 118), 89 mg (0.36 mmol) 1-(3,4-difluorobenzyl)-3,5-dimethyl-1H-pyrazol-4-amine (intermediate 4C) and 100 mg (0.31 mmol) 7-bromo-2-methylquinoline-4-carboxylic acid (intermediate 22A) were reacted to give after purification via preparative HPLC (method 3) 78 mg (42%) of the desired title compound.

¹H-NMR (400 MHz, DMSO d₆) δ (ppm)=2.14 (s, 3H), 2.19 (s, 3H), 5.25 (s, 2H), 7.00 (ddd, 1H), 7.20 (ddd, 1H), 7.43 (dt, 1H), 8.05 (dd, 1H), 8.20-8.27 (m, 2H), 8.53 (d, 1H), 10.13 (s, 1H).

### Example 190

### 6-bromo-N-[1-(3-chloro-4-fluorobenzyl)-3,5-dimethyl-1H-pyrazol-4-yl]-2-(trifluoromethyl)quinoline-4-carboxamide

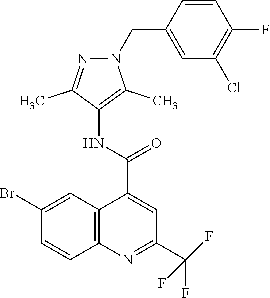 [SMILES: Cc1nn(Cc2ccc(F)c(Cl)c2)c(C)c1NC(=O)c1cc(C(F)(F)F)nc2ccc(Br)cc12]

In analogy to example 118), 95 mg (0.38 mmol) 1-(3-chloro-4-fluorobenzyl)-3,5-dimethyl-1H-pyrazol-4-amine (intermediate 28C) and 100 mg (0.31 mmol) 6-bromo-2-methylquinoline-4-carboxylic acid (intermediate 1A) were reacted to give after purification via preparative HPLC (method 3) 106 mg (58%) of the desired title compound.

¹H-NMR (400 MHz, DMSO d₆) δ (ppm)=2.15 (s, 3H), 2.20 (s, 3H), 5.26 (s, 2H), 7.14-7.21 (m, 1H), 7.36-7.45 (m, 2H), 8.11-8.16 (m, 1H), 8.22 (d, 1H), 8.28 (s, 1H), 8.50 (d, 1H), 10.17 (s, 1H).

### Example 191

### N-[1-(3-chloro-4-fluorobenzyl)-3,5-dimethyl-1H-pyrazol-4-yl]-6,7-difluoro-2-(trifluoromethyl)quinoline-4-carboxamide

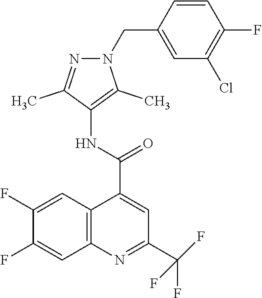 [SMILES: Cc1nn(Cc2ccc(F)c(Cl)c2)c(C)c1NC(=O)c1cc(C(F)(F)F)nc2cc(F)c(F)cc12]

In analogy to example 118), 110 mg (0.43 mmol) 1-(3-chloro-4-fluorobenzyl)-3,5-dimethyl-1H-pyrazol-4-amine (intermediate 28C) and 100 mg (0.36 mmol) 6,7-difluoro-2-(trifluoromethyl)quinoline-4-carboxylic acid (intermediate 19A) were reacted to give after purification via preparative HPLC (method 3) 119 mg (60%) of the desired title compound.

¹H-NMR (400 MHz, DMSO d₆) δ (ppm)=2.14 (s, 3H), 2.19 (s, 3H), 5.26 (s, 2H), 7.18 (ddd, 1H), 7.35-7.45 (m, 2H), 8.26 (dd, 1H), 8.30 (s, 1H), 8.40 (dd, 1H), 10.18 (s, 1H).

### Example 192

### N-[1-(4-fluorobenzyl)-3,5-dimethyl-1H-pyrazol-4-yl]-6-methoxyquinoline-4-carboxamide

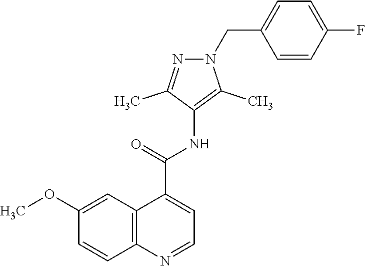 [SMILES: COc1ccc2nccc(C(=O)Nc3c(C)nn(Cc4ccc(F)cc4)c3C)c2c1]

In analogy to example 118), 130 mg (0.59 mmol) 1-(4-fluorobenzyl)-3,5-dimethyl-1H-pyrazol-4-amine (intermediate 1C) and 100 mg (0.49 mmol) 6-methoxyquinoline-4-carboxylic acid were reacted to give after purification via preparative HPLC (method 3) 106 mg (52%) of the desired title compound.

¹H-NMR (300 MHz, DMSO d₆) δ (ppm)=2.14 (s, 3H), 2.17 (s, 3H), 3.86 (s, 3H), 5.23 (s, 2H), 7.13-7.29 (m, 4H), 7.46-7.53 (m, 2H), 7.67 (d, 1H), 8.02 (d, 1H), 8.85 (d, 1H), 9.89 (s, 1H).

### Example 193

### 6,7-difluoro-N-[1-(4-fluorobenzyl)-5-methyl-3-(trifluoromethyl)-1H-pyrazol-4-yl]-2-(trifluoromethyl)quinoline-4-carboxamide

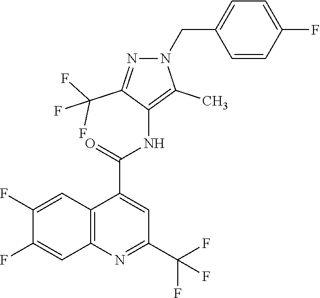 [SMILES: Cc1c(NC(=O)c2cc(C(F)(F)F)nc3cc(F)c(F)cc23)c(C(F)(F)F)nn1Cc1ccc(F)cc1]

In analogy to example 118), 95 mg (0.35 mmol) 1-(4-fluorobenzyl)-5-methyl-3-(trifluoromethyl)-1H-pyrazol-4-amine (intermediate 25C) and 80 mg (0.29 mmol) 6,7-difluoro-2-(trifluoromethyl)quinoline-4-carboxylic acid (intermediate 19A) were reacted to give after purification via preparative HPLC (method 4) 110 mg (66%) of the desired title compound.

¹H-NMR (300 MHz, DMSO d) δ (ppm)=2.29 (s, 3H), 5.46 (s, 2H), 7.17-7.35 (m, 4H), 8.14 (dd, 1H), 8.25 (s, 1H), 8.43 (dd, 1H), 10.52 (s, 1H).

### Example 194

### 6-bromo-N-[1-(4-fluorobenzyl)-3,5-dimethyl-1H-pyrazol-4-yl]-N-(2-methoxyethyl)-2-(trifluoromethyl)quinoline-4-carboxamide

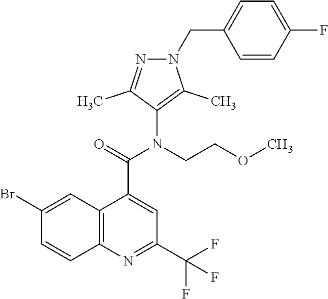 [SMILES: COCCN(C(=O)c1cc(C(F)(F)F)nc2ccc(Br)cc12)c1c(C)nn(Cc2ccc(F)cc2)c1C]

In analogy to example 176), 200 mg (0.38 mmol) 6-bromo-N-[1-(4-fluorobenzyl)-3,5-dimethyl-1H-pyrazol-4-yl]-2-(trifluoromethyl)quinoline-4-carboxamide (example 9) and 1.50 mL (0.46 mmol) 1-bromo-2-methoxyethane were reacted to give after purification via preparative HPLC (method 4) 56 mg (23%) of the desired title compound.

¹H-NMR (400 MHz, DMSO d₆) δ (ppm)=1.77 (s, 3H), 2.03 (s, 3H), 3.31 (s, 3H), 3.44-3.58 (m, 2H), 3.68-3.77 (m, 1H), 4.23-4.32 (m, 1H), 4.86-5.01 (m, 2H), 6.22 (dd, 2H), 6.83-6.91 (m, 2H), 7.82 (s, 1H), 8.04 (dd, 1H), 8.12 (d, 1H), 8.22 (d, 1H).

### Example 195

### N-[1-(cyclohexylmethyl)-3,5-dimethyl-1H-pyrazol-4-yl]-6,7-difluoro-2-(trifluoromethyl)quinoline-4-carboxamide

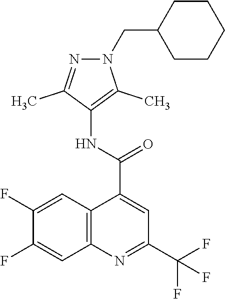 [SMILES: Cc1nn(CC2CCCCC2)c(C)c1NC(=O)c1cc(C(F)(F)F)nc2cc(F)c(F)cc12]

In analogy to example 118), 90 mg (0.43 mmol) 1-(cyclohexylmethyl)-3,5-dimethyl-1H-pyrazol-4-amine (intermediate 33C) and 100 mg (0.36 mmol) 6,7-difluoro-2-(trifluoromethyl)quinoline-4-carboxylic acid (intermediate 19A) were reacted to give after purification via preparative HPLC (method 4) 53 mg (30%) of the desired title compound.

¹H-NMR (300 MHz, DMSO d₆) δ (ppm)=0.88-1.27 (m, 5H), 1.47-1.85 (m, 6H), 2.10 (s, 3H), 2.19 (s, 3H), 3.80 (d, 2H), 8.20-8.30 (m, 2H), 8.41 (dd, 1H), 10.12 (s, 1H).

### Example 196

### N-[3,5-dimethyl-1-(pyridin-3-ylmethyl)-1H-pyrazol-4-yl]-6,7-difluoro-2-(trifluoromethyl)quinoline-4-carboxamide

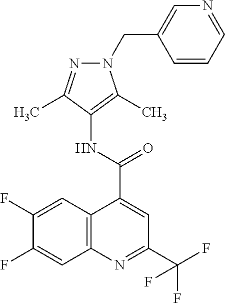 [SMILES: Cc1nn(Cc2cccnc2)c(C)c1NC(=O)c1cc(C(F)(F)F)nc2cc(F)c(F)cc12]

In analogy to example 118), 90 mg (0.45 mmol) 3,5-dimethyl-1-(pyridin-3-ylmethyl)-1H-pyrazol-4-amine (intermediate 17C) and 103 mg (0.37 mmol) 6,7-difluoro-2-(trifluoromethyl)quinoline-4-carboxylic acid (intermediate 19A) were reacted to give after purification via preparative HPLC (method 3) 25 mg (14%) of the desired title compound.

¹H-NMR (300 MHz, DMSO d₆) δ (ppm)=2.13 (s, 3H), 2.22 (s, 3H), 5.32 (s, 2H), 7.45 (dd, 1H), 7.64 (d, 1H), 8.20-8.32 (m, 2H), 8.41 (dd, 1H), 8.45-8.49 (m, 1H), 8.51-8.57 (m, 1H), 10.19 (s, 1H).

### Example 197

### 6-bromo-N-[1-(3,4-difluorobenzyl)-5-methyl-3-(trifluoromethyl)-1H-pyrazol-4-yl]-2-(trifluoromethyl)quinoline-4-carboxamide

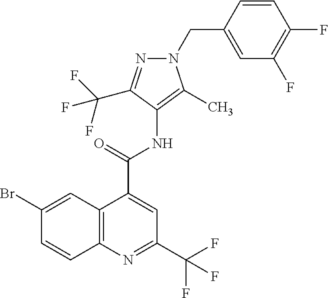 [SMILES: Cc1c(NC(=O)c2cc(C(F)(F)F)nc3ccc(Br)cc23)c(C(F)(F)F)nn1Cc1ccc(F)c(F)c1]

In analogy to example 118), 87 mg (0.30 mmol) 1-(3,4-difluorobenzyl)-5-methyl-3-(trifluoromethyl)-1H-pyrazol-4-amine (intermediate 23C) and 80 mg (0.25 mmol) 6-bromo-2-methylquinoline-4-carboxylic acid (intermediate 1A) were reacted to give after purification via preparative HPLC (method 4) 80 mg (52%) of the desired title compound.

¹H-NMR (400 MHz, DMSO d₆) δ (ppm)=2.31 (s, 3H), 5.47 (s, 2H), 7.08 (ddd, 1H), 7.34 (ddd, 1H), 7.48 (dt, 1H), 8.12-8.18 (m, 1H), 8.21-8.27 (m, 2H), 8.41 (d, 1H), 10.50 (s, 1H).

### Example 198

### N-[1-(4-fluorobenzyl)-3,5-dimethyl-1H-pyrazol-4-yl]-2-(morpholin-4-yl)quinoline-4-carboxamide

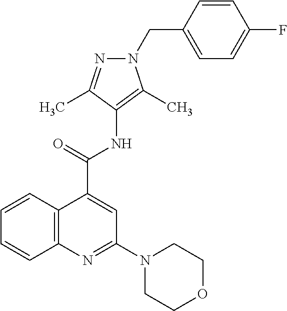 [SMILES: Cc1nn(Cc2ccc(F)cc2)c(C)c1NC(=O)c1cc(N2CCOCC2)nc2ccccc12]

In analogy to example 118), 93 mg (0.43 mmol) 1-(4-fluorobenzyl)-3,5-dimethyl-1H-pyrazol-4-amine (intermediate 1C) and 100 mg (0.39 mmol) 2-(morpholin-4-yl)quinoline-4-carboxylic acid were reacted to give after purification via preparative HPLC (method 3) 63 mg (33%) of the desired title compound.

¹H-NMR (300 MHz, DMSO d₆) δ (ppm)=2.12 (s, 3H), 2.16 (s, 3H), 3.72 (d, 8H), 5.23 (s, 2H), 7.14-7.26 (m, 4H), 7.30 (t, 1H), 7.39 (s, 1H), 7.54-7.67 (m, 2H), 7.89 (d, 1H), 9.80 (s, 1H).

### Example 199

### 6,8-dichloro-N-[1-(3,4-difluorobenzyl)-5-methyl-3-(trifluoromethyl)-1H-pyrazol-4-yl]-2-(trifluoromethyl)quinoline-4-carboxamide

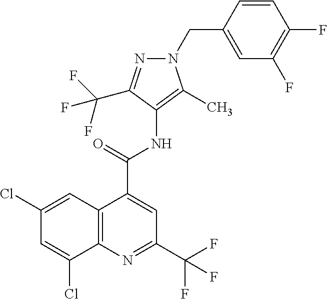 [SMILES: Cc1c(NC(=O)c2cc(C(F)(F)F)nc3c(Cl)cc(Cl)cc23)c(C(F)(F)F)nn1Cc1ccc(F)c(F)c1]

In analogy to example 118), 90 mg (0.31 mmol) 1-(3,4-difluorobenzyl)-5-methyl-3-(trifluoromethyl)-1H-pyrazol-4-amine (intermediate 23C) and 80 mg (0.26 mmol) 6,8-dichloro-2-(trifluoromethyl)quinoline-4-carboxylic acid (intermediate 11A) were reacted to give after purification via preparative HPLC (method 4) 68 mg (44%) of the desired title compound.

¹H-NMR (400 MHz, DMSO d₆) δ (ppm)=2.31 (s, 3H), 5.47 (s, 2H), 7.08 (ddd, 1H), 7.33 (ddd, 1H), 7.48 (dt, 1H), 8.19 (d, 1H), 8.37 (s, 1H), 8.40 (d, 1H), 10.54 (s, 1H).

### Example 200

### N-benzyl-6-bromo-N-[1-(4-fluorobenzyl)-3,5-dimethyl-1H-pyrazol-4-yl]-2-trifluoromethyl)quinoline-4-carboxamide

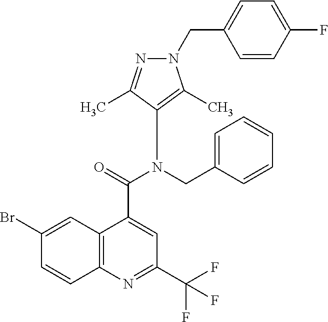 [SMILES: Cc1nn(Cc2ccc(F)cc2)c(C)c1N(Cc1ccccc1)C(=O)c1cc(C(F)(F)F)nc2ccc(Br)cc12]

In analogy to example 176), 200 mg (0.38 mmol) 6-bromo-N-[1-(4-fluorobenzyl)-3,5-dimethyl-1H-pyrazol-4-yl]-2-(trifluoromethyl)quinoline-4-carboxamide (example 9) and 55 μL (0.46 mmol) benzyl bromide were reacted to give after purification via preparative HPLC (method 4) 152 mg (61%) of the desired title compound.

¹H-NMR (300 MHz, DMSO d₆) δ (ppm)=1.42 (s, 3H), 1.81 (s, 3H), 4.77 (d, 1H), 4.85 (d, 2H), 5.21 (d, 1H), 6.15 (dd, 2H), 6.87 (t, 2H), 7.29-7.40 (m, 5H), 7.95 (s, 1H), 8.04 (dd, 1H), 8.12 (d, 1H), 8.22 (d, 1H).

### Example 201

### N-{1-[(6-cyanopyridin-3-yl)methyl]-3,5-dimethyl-1H-pyrazol-4-yl}-2,6-dimethylquinoline-4-carboxamide

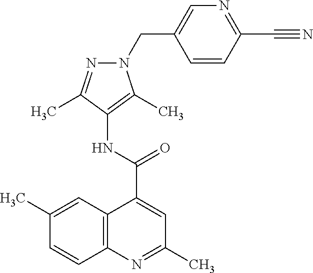 [SMILES: Cc1ccc2nc(C)cc(C(=O)Nc3c(C)nn(Cc4ccc(C#N)nc4)c3C)c2c1]

In analogy to example 118), 159 mg (0.60 mmol) 5-[(4-amino-3,5-dimethyl-1H-pyrazol-1-yl)methyl]pyridine-2-carbonitrile (intermediate 29C) and 100 mg (0.506 mmol) 2,6-dimethylquinoline-4-carboxylic acid were reacted to give after purification via preparative HPLC (method 3) 13 mg (5.7%) of the desired title compound.

¹H-NMR (600 MHz, DMSO d₆) δ (ppm)=2.14 (s, 3H), 2.21 (s, 3H), 2.50 (s, 3H), 2.75 (s, 3H), 5.43 (s, 2H), 7.66-7.73 (m, 2H), 7.76 (dd, 1H), 7.91 (s, 1H), 7.95 (d, 1H), 8.05 (d, 1H), 8.59 (d, 1H), 9.97 (s, 1H).

### Example 202

### N-(1-{4-[(2-hydroxyethyl)carbamoyl]benzyl}-3,5-dimethyl-1H-pyrazol-4-yl)-2, 6-dimethylquinoline-4-carboxamide

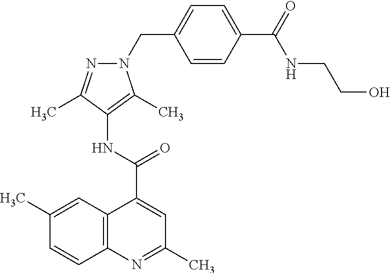 [SMILES: Cc1ccc2nc(C)cc(C(=O)Nc3c(C)nn(Cc4ccc(C(=O)NCCO)cc4)c3C)c2c1]

In analogy to example 118), 100 mg (0.35 mmol) 4-[(4-amino-3,5-dimethyl-1H-pyrazol-1-yl)methyl]-N-(2-hydroxyethyl)benzamide (intermediate 41C) and 58 mg (0.29 mmol) 2,6-dimethylquinoline-4-carboxylic acid were reacted to give after purification via preparative HPLC (method 3) 3.9 mg (2.7%) of the desired title compound.

¹H-NMR (300 MHz, DMSO d₆) δ (ppm)=2.14 (s, 3H), 2.16 (s, 3H), 2.48 (s, 3H), 2.69 (s, 3H), 3.49 (t, 3H), 5.30 (s, 2H), 7.19-7.27 (m, 2H), 7.52-7.64 (m, 2H), 7.77-7.84 (m, 2H), 7.85-7.93 (m, 2H), 8.37 (t, 1H), 9.85 (s, 1H).

### Example 203

### N-[1-(3,4-difluorobenzyl)-5-methyl-3-(trifluoromethyl)-1H-pyrazol-4-yl]-2,6-dimethylquinoline-4-carboxamide

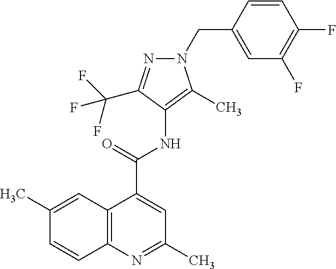 [SMILES: Cc1ccc2nc(C)cc(C(=O)Nc3c(C(F)(F)F)nn(Cc4ccc(F)c(F)c4)c3C)c2c1]

In analogy to example 118), 139 mg (0.48 mmol) 1-(3,4-difluorobenzyl)-5-methyl-3-(trifluoromethyl)-1H-pyrazol-4-amine (intermediate 23C) and 80 mg (0.40 mmol) 2,6-dimethylquinoline-4-carboxylic acid were reacted to give after purification via preparative HPLC (method 4) 135 mg (68%) of the desired title compound.

¹H-NMR (400 MHz, DMSO d₆) δ (ppm)=2.27 (s, 3H), 2.47 (s, 3H), 2.69 (s, 3H), 5.46 (s, 2H), 7.08 (ddd, 1H), 7.34 (ddd, 1H), 7.44-7.52 (m, 2H), 7.61 (dd, 1H), 7.82 (s, 1H), 7.89 (d, 1H), 10.18 (s, 1H).

### Example 204

### N-[1-(2,4-difluorobenzyl)-5-methyl-3-(trifluoromethyl)-1H-pyrazol-4-yl]-2,6-dimethylquinoline-4-carboxamide

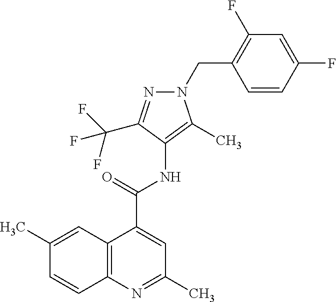 [SMILES: Cc1ccc2nc(C)cc(C(=O)Nc3c(C(F)(F)F)nn(Cc4ccc(F)cc4F)c3C)c2c1]

In analogy to example 118), 139 mg (0.48 mmol) 1-(2,4-difluorobenzyl)-5-methyl-3-(trifluoromethyl)-1H-pyrazol-4-amine (intermediate 24C) and 80 mg (0.40 mmol) 2,6-dimethylquinoline-4-carboxylic acid were reacted to give after purification via preparative HPLC (method 4) 133 mg (68%) of the desired title compound.

¹H-NMR (300 MHz, DMSO d₆) δ (ppm)=2.31 (s, 3H), 2.47 (s, 3H), 2.69 (s, 3H), 5.47 (s, 2H), 7.10-7.20 (m, 1H), 7.26-7.38 (m, 2H), 7.52 (s, 1H), 7.61 (dd, 1H), 7.81 (s, 1H), 7.90 (d, 1H), 10.19 (s, 1H).

### Example 205

### 6,8-dichloro-N-[1-(2,4-difluorobenzyl)-5-methyl-3-(trifluoromethyl)-1H-pyrazol-4-yl]-2-(trifluoromethyl)quinoline-4-carboxamide

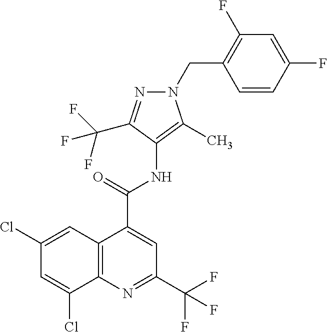 [SMILES: Cc1c(NC(=O)c2cc(C(F)(F)F)nc3c(Cl)cc(Cl)cc23)c(C(F)(F)F)nn1Cc1ccc(F)cc1F]

In analogy to example 118), 90 mg (0.31 mmol) 1-(2,4-difluorobenzyl)-5-methyl-3-(trifluoromethyl)-1H-pyrazol-4-amine (intermediate 24C) and 80 mg (0.26 mmol) 6,8-dichloro-2-(trifluoromethyl)quinoline-4-carboxylic acid (intermediate 11A) were reacted to give after purification via preparative HPLC (method 4) 76 mg (49%) of the desired title compound.

¹H-NMR (300 MHz, DMSO d₆) δ (ppm)=2.35 (s, 3H), 5.48 (s, 2H), 7.08-7.20 (m, 1H), 7.25-7.41 (m, 2H), 8.19 (d, 1H), 8.36-8.44 (m, 2H), 10.55 (s, 1H).

### Example 206

### N-[1-(cyclohexylmethyl)-3,5-dimethyl-1H-pyrazol-4-yl]-2,6-dimethylquinoline-4-carboxamide

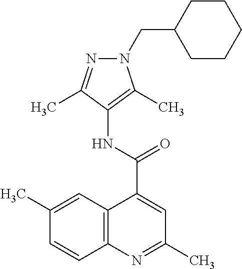 [SMILES: Cc1ccc2nc(C)cc(C(=O)Nc3c(C)nn(CC4CCCCC4)c3C)c2c1]

In analogy to example 118), 124 mg (0.60 mmol) 1-(cyclohexylmethyl)-3,5-dimethyl-1H-pyrazol-4-amine (intermediate 33C) and 100 mg (0.50 mmol) 2,6-dimethylquinoline-4-carboxylic acid were reacted to give after purification via preparative HPLC (method 3) 123 mg (60%) of the desired title compound.

¹H-NMR (300 MHz, DMSO d₆) δ (ppm)=0.88-1.29 (m, 5H), 1.48-1.84 (m, 6H), 2.10 (s, 3H), 2.17 (s, 3H), 2.48 (s, 3H), 2.70 (s, 3H), 3.79 (d, 2H), 7.57-7.67 (m, 2H), 7.85-7.94 (m, 2H), 9.82 (s, 1H).

### Example 207

### N-[1-(3,4-difluorobenzyl)-5-methyl-3-(trifluoromethyl)-1H-pyrazol-4-yl]-6,7-difluoro-2-(trifluoromethyl)quinoline-4-carboxamide

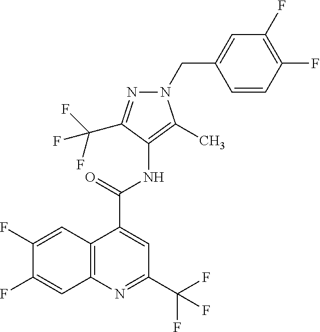 [SMILES: Cc1c(NC(=O)c2cc(C(F)(F)F)nc3cc(F)c(F)cc23)c(C(F)(F)F)nn1Cc1ccc(F)c(F)c1]

In analogy to example 118), 109 mg (0.35 mmol) 1-(3,4-difluorobenzyl)-5-methyl-3-(trifluoromethyl)-1H-pyrazol-4-amine (intermediate 23C) and 80 mg (0.29 mmol) 6,7-difluoro-2-(trifluoromethyl)quinoline-4-carboxylic acid (intermediate 19A) were reacted to give after purification via preparative HPLC (method 4) 117 mg (70%) of the desired title compound.

¹H-NMR (400 MHz, DMSO d₆) δ (ppm)=2.30 (s, 3H), 5.47 (s, 2H), 7.07 (ddd, 1H), 7.33 (ddd, 1H), 7.48 (dt, 1H), 8.14 (dd, 1H), 8.25 (s, 1H), 8.42 (dd, 1H), 10.52 (s, 1H).

### Example 208

### N-[3,5-dimethyl-1-(pyridin-3-ylmethyl)-1H-pyrazol-4-yl]-2,6-dimethylquinoline-4-carboxamide

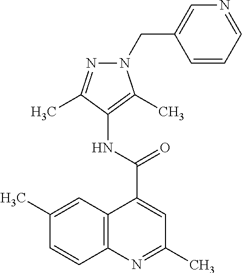 [SMILES: Cc1ccc2nc(C)cc(C(=O)Nc3c(C)nn(Cc4cccnc4)c3C)c2c1]

In analogy to example 118), 90 mg (0.45 mmol) 3,5-dimethyl-1-(pyridin-3-ylmethyl)-1H-pyrazol-4-amine (intermediate 17C) and 75 mg (0.37 mmol) 2,6-dimethylquinoline-4-carboxylic acid were reacted to give after purification via preparative HPLC (method 3) 68 mg (45%) of the desired title compound.

¹H-NMR (400 MHz, DMSO d₆) δ (ppm)=2.14 (s, 3H), 2.24 (s, 3H), 2.51 (s, 3H), 2.77 (s, 3H), 5.38 (s, 2H), 7.68 (dd, 1H), 7.71-7.77 (m, 2H), 7.88-7.93 (m, 2H), 7.97 (d, 1H), 8.60 (d, 1H), 8.66 (dd, 1H), 10.00 (s, 1H).

### Example 209

### N⁴-[1-(4-fluorobenzyl)-3,5-dimethyl-1H-pyrazol-4-yl]-N²,N²-dimethylquinoline-2,4-dicarboxamide

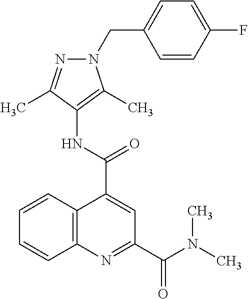 [SMILES: Cc1nn(Cc2ccc(F)cc2)c(C)c1NC(=O)c1cc(C(=O)N(C)C)nc2ccccc12]

In analogy to example 118), 59 mg (0.27 mmol) 1-(4-fluorobenzyl)-3,5-dimethyl-1H-pyrazol-4-amine (intermediate 1C) and 68 mg (0.22 mmol, puritiy 80%) 2-(dimethylcarbamoyl)quinoline-4-carboxylic acid (intermediate 7A) were reacted to give after purification via preparative HPLC (method 3) 67 mg (62%) of the desired title compound.

¹H-NMR (400 MHz, DMSO d₆) δ (ppm)=2.13 (s, 3H), 2.17 (s, 3H), 3.04 (s, 3H), 3.09 (s, 3H), 5.23 (s, 2H), 7.14-7.27 (m, 4H), 7.76 (ddd, 1H), 7.81 (s, 1H), 7.89 (ddd, 1H), 8.12 (d, 1H), 8.19 (d, 1H), 10.00 (s, 1H).

### Example 210

### N-[1-(2,4-difluorobenzyl)-5-methyl-3-(trifluoromethyl)-1H-pyrazol-4-yl]-6,7-difluoro-2-(trifluoromethyl)quinoline-4-carboxamide

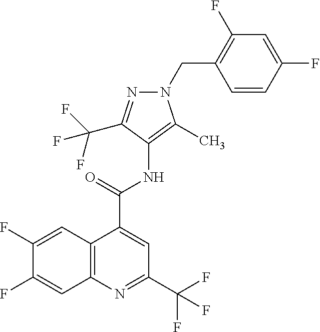 [SMILES: Cc1c(NC(=O)c2cc(C(F)(F)F)nc3cc(F)c(F)cc23)c(C(F)(F)F)nn1Cc1ccc(F)cc1F]

In analogy to example 118), 101 mg (0.35 mmol) 1-(2,4-difluorobenzyl)-5-methyl-3-(trifluoromethyl)-1H-pyrazol-4-amine (intermediate 24C) and 80 mg (0.29 mmol, purity 80%) 6,7-difluoro-2-(trifluoromethyl)quinoline-4-carboxylic acid (intermediate 19A) were reacted to give after purification via preparative HPLC (method 4) 111 mg (68%) of the desired title compound.

¹H-NMR (300 MHz, DMSO d₆) δ (ppm)=2.34 (s, 3H), 5.48 (s, 2H), 7.11-7.20 (m, 1H), 7.26-7.39 (m, 2H), 8.14 (dd, 1H), 8.26 (s, 1H), 8.43 (dd, 1H), 10.53 (s, 1H).

### Example 211

### N-[1-(4-fluorobenzyl)-3,5-dimethyl-1H-pyrazol-4-yl]quinoline-4-carboxamide

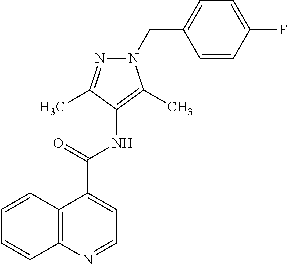 [SMILES: Cc1nn(Cc2ccc(F)cc2)c(C)c1NC(=O)c1ccnc2ccccc12]

In analogy to example 118), 152 mg (0.69 mmol) 1-(4-fluorobenzyl)-3,5-dimethyl-1H-pyrazol-4-amine (intermediate 1C) and 100 mg (0.58 mmol) quinoline-4-carboxylic acid were reacted to give after purification via preparative HPLC (method 4) 162 mg (68%) of the desired title compound.

¹H-NMR (300 MHz, DMSO d₆) δ (ppm)=2.13 (s, 3H), 2.17 (s, 3H), 5.24 (s, 2H), 7.13-7.28 (m, 4H), 7.66-7.74 (m, 2H), 7.83 (td, 1H), 8.11 (d, 1H), 8.17 (d, 1H), 9.02 (d, 1H), 9.92 (s, 1H).

### Example 212

### 6-chloro-N⁴-[1-(4-cyanobenzyl)-3,5-dimethyl-1H-pyrazol-4-yl]-7-fluoroquinoline-2,4-dicarboxamide

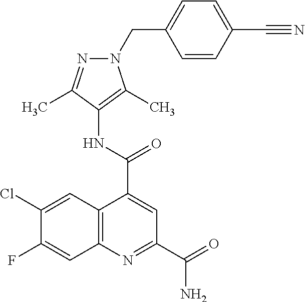 [SMILES: Cc1nn(Cc2ccc(C#N)cc2)c(C)c1NC(=O)c1cc(C(N)=O)nc2cc(F)c(Cl)cc12]

In analogy to example 118), 96 mg (0.42 mmol) 4-[(4-amino-3,5-dimethyl-1H-pyrazol-1-yl)methyl]benzonitrile (intermediate 8C) and 95 mg (0.35 mmol) 2-carbamoyl-6-chloro-7-fluoroquinoline-4-carboxylic acid (intermediate 32A) were reacted to give after purification via preparative HPLC (method 3) 8.7 mg (4.6%) of the desired title compound.

¹H-NMR (300 MHz, DMSO d₆) δ (ppm)=2.13 (s, 3H), 2.16 (s, 3H), 5.37 (s, 2H), 7.28-7.36 (m, 2H), 7.79-7.87 (m, 2H), 7.98 (br. s., 1H), 8.12 (d, 1H), 8.33-8.42 (m, 2H), 8.51 (d, 1H), 10.22 (s, 1H).

### Example 213

### 6-chloro-N⁴-[1-(4-cyanobenzyl)-5-methyl-3-(trifluoromethyl)-1H-pyrazol-4-yl]-7-fluoroquinoline-2,4-dicarboxamide

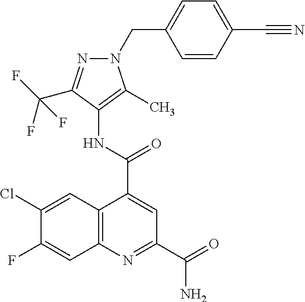 [SMILES: Cc1c(NC(=O)c2cc(C(N)=O)nc3cc(F)c(Cl)cc23)c(C(F)(F)F)nn1Cc1ccc(C#N)cc1]

In analogy to example 118), 119 mg (0.42 mmol) 4-{[4-amino-5-methyl-3-(trifluoromethyl)-1H-pyrazol-1-yl]methyl}benzonitrile (intermediate 26C) and 95 mg (0.35 mmol) 2-carbamoyl-6-chloro-7-fluoroquinoline-4-carboxylic acid (intermediate 32A) were reacted to give after purification via preparative HPLC (method 3) 72 mg (37%) of the desired title compound.

¹H-NMR (300 MHz, DMSO d₆) δ (ppm)=2.26 (s, 3H), 5.60 (s, 2H), 7.35-7.43 (m, 2H), 7.85-7.92 (m, 2H), 7.99 (br. s., 1H), 8.14 (d, 1H), 8.36 (s, 1H), 8.38 (br. s., 1H), 8.43 (d, 1H), 10.54 (s, 1H).

### Example 214

### 6-chloro-7-fluoro-N⁴-[1-(4-methoxybenzyl)-3,5-dimethyl-1H-pyrazol-4-yl]quinoline-2,4-dicarboxamide

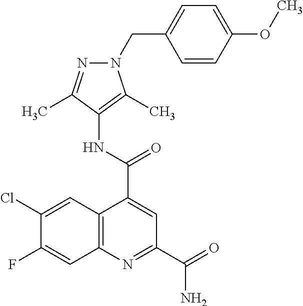 [SMILES: COc1ccc(Cn2nc(C)c(NC(=O)c3cc(C(N)=O)nc4cc(F)c(Cl)cc34)c2C)cc1]

In analogy to example 118), 98 mg (0.42 mmol) 1-(4-methoxybenzyl)-3,5-dimethyl-1H-pyrazol-4-amine (intermediate 11C) and 95 mg (0.35 mmol) 2-carbamoyl-6-chloro-7-fluoroquinoline-4-carboxylic acid (intermediate 32A) were reacted to give after purification via preparative HPLC (method 3) 9.1 mg (4.8%) of the desired title compound.

¹H-NMR (300 MHz, DMSO d₆) δ (ppm)=2.12 (s, 3H), 2.16 (s, 3H), 3.73 (s, 3H), 5.16 (s, 2H), 6.87-6.94 (m, 2H), 7.12-7.19 (m, 2H), 7.97 (br. s., 1H), 8.12 (d, 1H), 8.34-8.40 (m, 2H), 8.50 (d, 1H), 10.16 (s, 1H).

### Example 215

### 6-chloro-N⁴-[1-(2-cyanobenzyl)-3,5-dimethyl-1H-pyrazol-4-yl]-7-fluoroquinoline-2,4-dicarboxamide

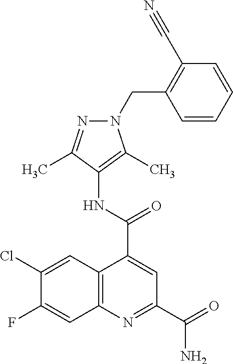 [SMILES: Cc1nn(Cc2ccccc2C#N)c(C)c1NC(=O)c1cc(C(N)=O)nc2cc(F)c(Cl)cc12]

In analogy to example 118), 96 mg (0.42 mmol) 2-[(4-amino-3,5-dimethyl-1H-pyrazol-1-yl)methyl]benzonitrile (intermediate 10C) and 95 mg (0.35 mmol) 2-carbamoyl-6-chloro-7-fluoroquinoline-4-carboxylic acid (intermediate 32A) were reacted to give after purification via preparative HPLC (method 3) 14 mg (6.8%) of the desired title compound.

¹H-NMR (300 MHz, DMSO d₆) δ (ppm)=2.12 (s, 3H), 2.24 (s, 3H), 5.43 (s, 2H), 7.11 (d, 1H), 7.47-7.56 (m, 1H), 7.71 (td, 1H), 7.89 (dd, 1H), 7.98 (br. s., 1H), 8.13 (d, 1H), 8.34-8.42 (m, 2H), 8.52 (d, 1H), 10.25 (s, 1H).

### Example 216

### N⁴-[1-(4-cyanobenzyl)-5-methyl-3-(trifluoromethyl)-1H-pyrazol-4-yl]-6,7-difluoroquinoline-2,4-dicarboxamide

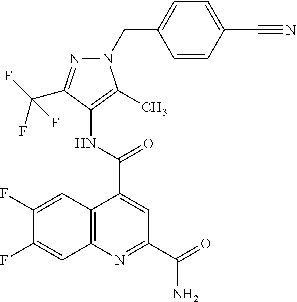 [SMILES: Cc1c(NC(=O)c2cc(C(N)=O)nc3cc(F)c(F)cc23)c(C(F)(F)F)nn1Cc1ccc(C#N)cc1]

In analogy to example 118), 133 mg (0.48 mmol) 4-{[4-amino-5-methyl-3-(trifluoromethyl)-1H-pyrazol-1-yl]methyl}benzonitrile (intermediate 26C) and 100 mg (0.40 mmol) 2-carbamoyl-6,7-difluoroquinoline-4-carboxylic acid (intermediate 3A) were reacted to give after purification via preparative HPLC (method 3) 20 mg (9.4%) of the desired title compound.

¹H-NMR (300 MHz, DMSO d₆) δ (ppm)=2.26 (s, 3H), 5.60 (s, 2H), 7.34-7.42 (m, 2H), 7.85-7.92 (m, 2H), 7.99 (s, 1H), 8.09-8.22 (m, 1H), 8.34-8.41 (m, 2H), 10.54 (s, 1H).

### Example 217

### 6-bromo-N-{3,5-dimethyl-1-[4-(methylsulfonyl)benzyl]-1H-pyrazol-4-yl}-2-(trifluoromethyl)quinoline-4-carboxamide

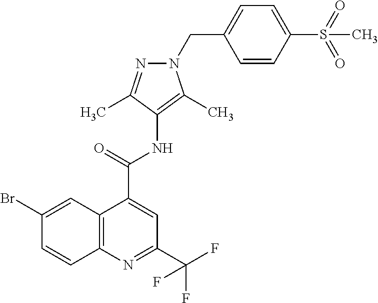 [SMILES: Cc1nn(Cc2ccc(S(C)(=O)=O)cc2)c(C)c1NC(=O)c1cc(C(F)(F)F)nc2ccc(Br)cc12]

In analogy to example 118), 112 mg (0.40 mmol) 3,5-dimethyl-1-[4-(methylsulfonyl)benzyl]-1H-pyrazol-4-amine (intermediate 37C) and 107 mg (0.33 mmol) 6-bromo-2-(trifluoromethyl)quinoline-4-carboxylic acid (intermediate 1A) were reacted to give after purification via preparative HPLC (method 3) 105 mg (52%) of the desired title compound.

¹H-NMR (400 MHz, DMSO d₆) δ (ppm)=2.15 (s, 3H), 2.20 (s, 3H), 3.19 (s, 3H), 5.39 (s, 2H), 7.42 (d, 2H), 7.92 (d, 2H), 8.14 (dd, 1H), 8.22 (d, 1H), 8.29 (s, 1H), 8.50 (d, 1H), 10.18 (s, 1H).

### Example 218

### 6-bromo-N-[1-(4-cyano-3-fluorobenzyl)-3,5-dimethyl-1H-pyrazol-4-yl]-2-(trifluoromethyl)quinoline-4-carboxamide

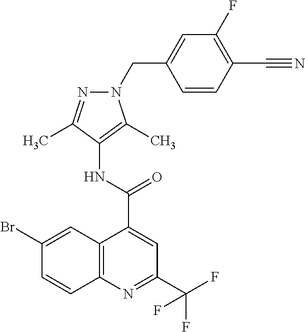 [SMILES: Cc1nn(Cc2ccc(C#N)c(F)c2)c(C)c1NC(=O)c1cc(C(F)(F)F)nc2ccc(Br)cc12]

In analogy to example 118), 92 mg (0.38 mmol) 4-[(4-amino-3,5-dimethyl-1H-pyrazol-1-yl)methyl]-2-fluorobenzonitrile (intermediate 38C) and 100 mg (0.31 mmol) 6-bromo-2-(trifluoromethyl)quinoline-4-carboxylic acid (intermediate 1A) were reacted to give after purification via preparative HPLC (method 3) 74 mg (42%) of the desired title compound.

¹H-NMR (300 MHz, DMSO d₆) δ (ppm)=2.13 (s, 3H), 2.23 (s, 3H), 5.39 (s, 2H), 7.15 (t, 1H), 7.71 (dd, 1H), 7.90 (dd, 1H), 8.14 (dd, 1H), 8.22 (d, 1H), 8.29 (s, 1H), 8.50 (d, 1H), 10.19 (s, 1H).

### Example 219

### N⁴-[1-(4-cyanobenzyl)-5-methyl-3-(trifluoromethyl)-1H-pyrazol-4-yl]-6-fluoroquinoline-2,4-dicarboxamide

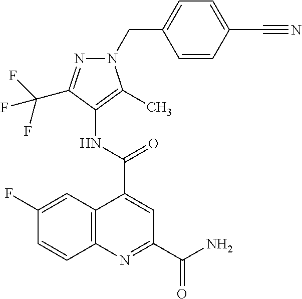 [SMILES: Cc1c(NC(=O)c2cc(C(N)=O)nc3ccc(F)cc23)c(C(F)(F)F)nn1Cc1ccc(C#N)cc1]

In analogy to example 118), 145 mg (0.52 mmol) 4-{[4-amino-5-methyl-3-(trifluoromethyl)-1H-pyrazol-1-yl]methyl}benzonitrile (intermediate 26C) and 125 mg (0.43 mmol, purity 81%) 6-fluoro-2-carbamoylquinoline-4-carboxylic acid (intermediate 8A) were reacted to give after purification via preparative HPLC (method 3) 97 mg (44%) of the desired title compound.

¹H-NMR (300 MHz, DMSO d₆) δ (ppm)=2.26 (s, 3H), 5.60 (s, 2H), 7.39 (d, 2H), 7.84-7.95 (m, 5H), 8.25-8.33 (m, 1H), 8.36 (s, 1H), 8.39 (br. s., 1H), 10.47 (s, 1H).

### Example 220

### N⁴-{1-[(2-cyanopyridin-3-yl)methyl]-3,5-dimethyl-1H-pyrazol-4-yl}-6,7-difluoroquinoline-2,4-dicarboxamide

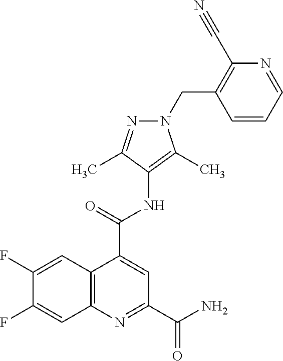 [SMILES: Cc1nn(Cc2cccnc2C#N)c(C)c1NC(=O)c1cc(C(N)=O)nc2cc(F)c(F)cc12]

In analogy to example 118), 100 mg (0.44 mmol) 3-[(4-amino-3,5-dimethyl-1H-pyrazol-1-yl)methyl]pyridine-2-carbonitrile (intermediate 39C) and 92 mg (0.37 mmol) 2-carbamoyl-6,7-difluoroquinoline-4-carboxylic acid (intermediate 3A) were reacted to give after purification via preparative HPLC (method 3) 48 mg (27%) of the desired title compound.

¹H-NMR (300 MHz, DMSO d₆) δ (ppm)=2.10 (s, 3H), 2.27 (s, 3H), 5.47 (s, 2H), 7.61 (dd, 1H), 7.76 (dd, 1H), 7.97 (s, 1H), 8.11-8.29 (m, 2H), 8.37 (s, 1H), 8.40 (s, 1H), 8.71 (dd, 1H), 10.25 (s, 1H).

### Example 221

### N⁴-[1-(2-cyanobenzyl)-3,5-dimethyl-1H-pyrazol-4-yl]-6-fluoroquinoline-2,4-dicarboxamide

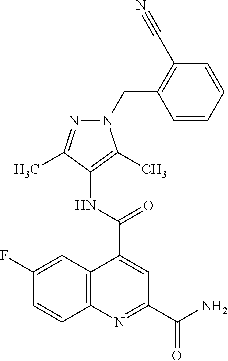 [SMILES: Cc1nn(Cc2ccccc2C#N)c(C)c1NC(=O)c1cc(C(N)=O)nc2ccc(F)cc12]

In analogy to example 118), 117 mg (0.52 mmol) 2-[(4-amino-3,5-dimethyl-1H-pyrazol-1-yl)methyl]benzonitrile (intermediate 10C) and 125 mg (0.43 mmol, purity 81%) 6-fluoro-2-carbamoylquinoline-4-carboxylic acid (intermediate 8A) were reacted to give after purification via preparative HPLC (method 3) 96 mg (48%) of the desired title compound.

¹H-NMR (300 MHz, DMSO d₆) δ (ppm)=2.12 (s, 3H), 2.24 (s, 3H), 5.43 (s, 2H), 7.11 (d, 1H), 7.47-7.57 (m, 1H), 7.66-7.75 (m, 1H), 7.83-7.94 (m, 3H), 7.99 (dd, 1H), 8.28 (dd, 1H), 8.35-8.43 (m, 2H), 10.18 (s, 1H).

### Example 222

### N⁴-{1-[(3-cyanopyridin-2-yl)methyl]-3,5-dimethyl-1H-pyrazol-4-yl}-6,7-difluoroquinoline-2,4-dicarboxamide

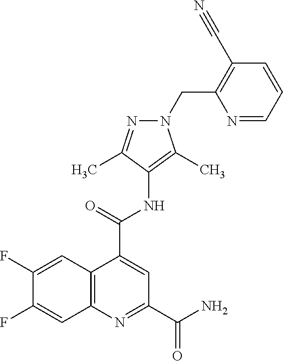 [SMILES: Cc1nn(Cc2ncccc2C#N)c(C)c1NC(=O)c1cc(C(N)=O)nc2cc(F)c(F)cc12]

In analogy to example 118), 100 mg (0.44 mmol) 2-[(4-amino-3,5-dimethyl-1H-pyrazol-1-yl)methyl]nicotinonitrile (intermediate 40C) and 92 mg (0.37 mmol) 2-carbamoyl-6,7-difluoroquinoline-4-carboxylic acid (intermediate 3A) were reacted to give after purification via preparative HPLC (method 3) 40 mg (22%) of the desired title compound.

¹H-NMR (400 MHz, DMSO d) δ (ppm)=2.06 (s, 3H), 2.30 (s, 3H), 5.52 (s, 2H), 7.57 (dd, 1H), 7.95 (s, 1H), 8.13-8.26 (m, 2H), 8.34-8.41 (m, 3H), 8.79 (dd, 1H), 10.20 (s, 1H).

### Example 223

### N⁴-{1-[(2-cyanopyridin-3-yl)methyl]-3,5-dimethyl-1H-pyrazol-4-yl}quinoline-2,4-dicarboxamide

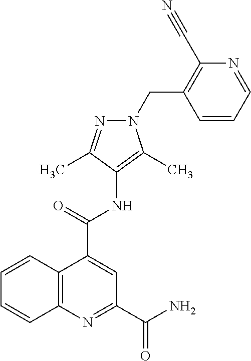 [SMILES: Cc1nn(Cc2cccnc2C#N)c(C)c1NC(=O)c1cc(C(N)=O)nc2ccccc12]

In analogy to example 118), 100 mg (0.44 mmol) 3-[(4-amino-3,5-dimethyl-1H-pyrazol-1-yl)methyl]pyridine-2-carbonitrile (intermediate 39C) and 79 mg (0.37 mmol) 2-carbamoylquinoline-4-carboxylic acid (intermediate 4A) were reacted to give after purification via preparative HPLC (method 3) 46 mg (28%) of the desired title compound.

¹H-NMR (400 MHz, DMSO d) δ (ppm)=2.12 (s, 3H), 2.29 (s, 3H), 5.48 (s, 2H), 7.61 (dd, 1H), 7.73-7.86 (m, 2H), 7.88-7.99 (m, 2H), 8.21 (d, 1H), 8.26 (d, 1H), 8.29 (s, 1H), 8.39 (br. s., 1H), 8.71 (dd, 1H), 10.13 (s, 1H).

### Example 224

### N⁴-{3,5-dimethyl-1-[4-(methylsulfonyl)benzyl]-1H-pyrazol-4-yl}quinoline-2,4-dicarboxamide

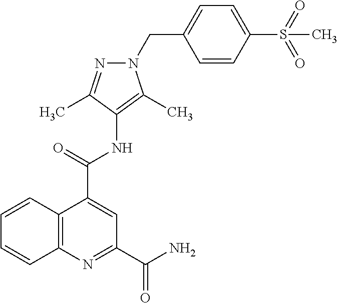 [SMILES: Cc1nn(Cc2ccc(S(C)(=O)=O)cc2)c(C)c1NC(=O)c1cc(C(N)=O)nc2ccccc12]

In analogy to example 118), 112 mg (0.40 mmol) 3,5-dimethyl-1-[4-(methylsulfonyl)benzyl]-1H-pyrazol-4-amine (intermediate 37C) and 72 mg (0.33 mmol) 2-carbamoylquinoline-4-carboxylic acid (intermediate 4A) were reacted to give after purification via preparative HPLC (method 3) 70 mg (42%) of the desired title compound.

¹H-NMR (400 MHz, DMSO d₆) δ (ppm)=2.15 (s, 3H), 2.20 (s, 3H), 3.19 (s, 3H), 5.39 (s, 2H), 7.43 (d, 2H), 7.77-7.84 (m, 1H), 7.86-7.96 (m, 4H), 8.20 (d, 1H), 8.25 (d, 1H), 8.27 (s, 1H), 8.35-8.40 (m, 1H), 10.08 (s, 1H).

### Example 225

### N⁴-{1-[(6-cyanopyridin-3-yl)methyl]-3,5-dimethyl-1H-pyrazol-4-yl}quinoline-2,4-dicarboxamide

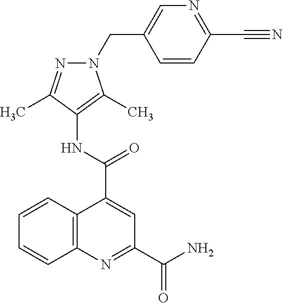 [SMILES: Cc1nn(Cc2ccc(C#N)nc2)c(C)c1NC(=O)c1cc(C(N)=O)nc2ccccc12]

In analogy to example 118), 105 mg (0.46 mmol) 5-[(4-amino-3,5-dimethyl-1H-pyrazol-1-yl)methyl]pyridine-2-carbonitrile (intermediate 29C) and 91 mg (0.42 mmol) 2-carbamoylquinoline-4-carboxylic acid (intermediate 4A) were reacted to give after purification via preparative HPLC (method 3) 28 mg (15%) of the desired title compound.

¹H-NMR (300 MHz, DMSO d₆) δ (ppm)=2.14 (s, 3H), 2.22 (s, 3H), 5.43 (s, 2H), 7.72-7.85 (m, 2H), 7.86-7.97 (m, 2H), 8.04 (d, 1H), 8.17-8.29 (m, 3H), 8.37 (br. s., 1H), 8.60 (d, 1H), 10.09 (s, 1H).

### Example 226

### 6,7-difluoro-N⁴-[1-(4-methoxybenzyl)-3,5-dimethyl-1H-pyrazol-4-yl]quinoline-2,4-dicarboxamide

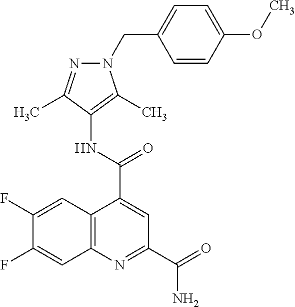 [SMILES: COc1ccc(Cn2nc(C)c(NC(=O)c3cc(C(N)=O)nc4cc(F)c(F)cc34)c2C)cc1]

In analogy to example 118), 105 mg (0.46 mmol) 1-(4-methoxybenzyl)-3,5-dimethyl-1H-pyrazol-4-amine (intermediate 11C) and 91 mg (0.42 mmol) 2-carbamoyl-6,7-difluoroquinoline-4-carboxylic acid (intermediate 3A) were reacted to give after filtration of the reaction mixture a solid which was used without further purification and yielded 128 mg (62%) of the desired title compound.

¹H-NMR (400 MHz, DMSO d₆) δ (ppm)=2.11 (s, 3H), 2.16 (s, 3H), 3.72 (s, 3H), 5.16 (s, 2H), 6.87-6.94 (m, 2H), 7.12-7.18 (m, 2H), 7.95 (s, 1H), 8.15 (dd, 1H), 8.22 (dd, 1H), 8.35 (s, 1H), 8.37 (s, 1H), 10.14 (s, 1H).

### Example 227

### 6-fluoro-N⁴-[1-(4-methoxybenzyl)-3,5-dimethyl-1H-pyrazol-4-yl]quinoline-2,4-dicarboxamide

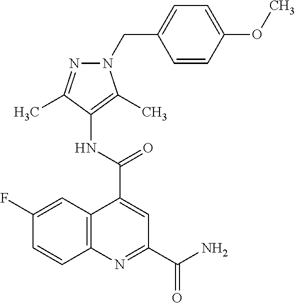 [SMILES: COc1ccc(Cn2nc(C)c(NC(=O)c3cc(C(N)=O)nc4ccc(F)cc34)c2C)cc1]

In analogy to example 118), 120 mg (0.52 mmol) 1-(4-methoxybenzyl)-3,5-dimethyl-1H-pyrazol-4-amine (intermediate 11C) and 125 mg (0.43 mmol, purity 81%) 6-fluoro-2-carbamoylquinoline-4-carboxylic acid (intermediate 8A) were reacted to give after purification via preparative HPLC (method 3) 95 mg (47%) of the desired title compound.

¹H-NMR (300 MHz, DMSO d₆) δ (ppm)=2.12 (s, 3H), 2.16 (s, 3H), 3.73 (s, 3H), 5.16 (s, 2H), 6.86-6.95 (m, 2H), 7.11-7.19 (m, 2H), 7.81-7.92 (m, 2H), 7.97 (dd, 1H), 8.27 (dd, 1H), 8.35-8.41 (m, 2H), 10.09 (s, 1H).

### Example 228

### N⁴-[1-(4-cyanobenzyl)-3,5-dimethyl-1H-pyrazol-4-yl]-6-fluoroquinoline-2,4-dicarboxamide

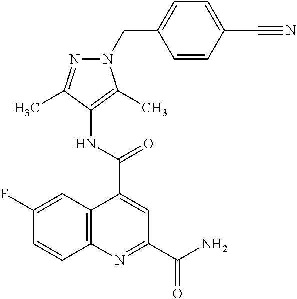 [SMILES: Cc1nn(Cc2ccc(C#N)cc2)c(C)c1NC(=O)c1cc(C(N)=O)nc2ccc(F)cc12]

In analogy to example 118), 117 mg (0.52 mmol) 4-[(4-amino-3,5-dimethyl-1H-pyrazol-1-yl)methyl]benzonitrile (intermediate 8C) and 125 mg (0.43 mmol, purity 81%) 6-fluoro-2-carbamoylquinoline-4-carboxylic acid (intermediate 8A) were reacted to give after purification via preparative HPLC (method 3) 115 mg (57%) of the desired title compound.

¹H-NMR (300 MHz, DMSO d₆) δ (ppm)=2.14 (s, 3H), 2.16 (s, 3H), 5.37 (s, 2H), 7.32 (d, 2H), 7.80-7.94 (m, 4H), 7.98 (dd, 1H), 8.28 (dd, 1H), 8.34-8.42 (m, 2H), 10.15 (s, 1H).

### Example 229

### 6-fluoro-N⁴-[1-(4-fluorobenzyl)-3,5-dimethyl-1H-pyrazol-4-yl]quinoline-2,4-dicarboxamide

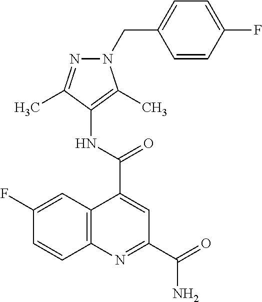 [SMILES: Cc1nn(Cc2ccc(F)cc2)c(C)c1NC(=O)c1cc(C(N)=O)nc2ccc(F)cc12]

In analogy to example 118), 105 mg (0.48 mmol) 1-(4-fluorobenzyl)-3,5-dimethyl-1H-pyrazol-4-amine (intermediate 1C) and 156 mg (0.40 mmol, purity 60%) 6-fluoro-2-carbamoylquinoline-4-carboxylic acid (intermediate 8A) were reacted to give after purification via preparative HPLC (method 3) 57 mg (31%) of the desired title compound.

¹H-NMR (300 MHz, DMSO d₆) δ (ppm)=2.12 (s, 3H), 2.17 (s, 3H), 5.24 (s, 2H), 7.14-7.29 (m, 4H), 7.83-8.01 (m, 3H), 8.27 (dd, 1H), 8.35-8.43 (m, 2H), 10.13 (s, 1H).

### Example 230

### N⁴-[1-(4-cyanobenzyl)-3,5-dimethyl-1H-pyrazol-4-yl]-6,7-difluoroquinoline-2,4-dicarboxamide

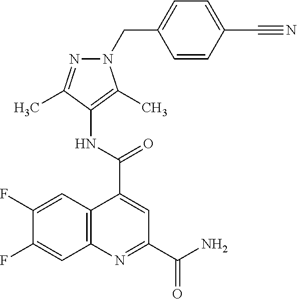 [SMILES: Cc1nn(Cc2ccc(C#N)cc2)c(C)c1NC(=O)c1cc(C(N)=O)nc2cc(F)c(F)cc12]

In analogy to example 118), 105 mg (0.46 mmol) 4-[(4-amino-3,5-dimethyl-1H-pyrazol-1-yl)methyl]benzonitrile (intermediate 8C) and 91 mg (0.42 mmol) 2-carbamoyl-6,7-difluoroquinoline-4-carboxylic acid (intermediate 3A) were reacted to give after purification using a Biotage chromatography system (10 g snap KP-Sil column, hexane/90-100% ethyl acetate, then ethyl acetate/0-75% methanol) a crude product, which was purified via HPLC (method 3) and yielded 46 mg (34%) of the desired title compound.

¹H-NMR (400 MHz, DMSO d₆) δ (ppm)=2.13 (s, 3H), 2.16 (s, 3H), 5.37 (s, 2H), 7.29-7.35 (m, 2H), 7.81-7.87 (m, 2H), 7.95 (s, 1H), 8.16 (dd, 1H), 8.24 (dd, 1H), 8.35 (s, 1H), 8.39 (s, 1H), 10.20 (s, 1H).

### Example 231

### N⁴-{3,5-dimethyl-1-[4-(methylsulfonyl)benzyl]-1H-pyrazol-4-yl}-6,7-difluoroquinoline-2,4-dicarboxamide

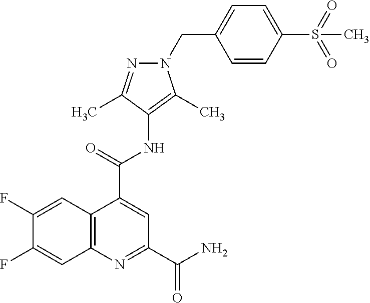 [SMILES: Cc1nn(Cc2ccc(S(C)(=O)=O)cc2)c(C)c1NC(=O)c1cc(C(N)=O)nc2cc(F)c(F)cc12]

In analogy to example 118), 112 mg (0.40 mmol) 3,5-dimethyl-1-[4-(methylsulfonyl)benzyl]-1H-pyrazol-4-amine (intermediate 37C) and 84 mg (0.33 mmol) 2-carbamoyl-6,7-difluoroquinoline-4-carboxylic acid (intermediate 3A) were reacted to give after purification via preparative HPLC (method 3) 45 mg (25%) of the desired title compound.

¹H-NMR (300 MHz, DMSO d₆) δ (ppm)=2.13 (s, 3H), 2.18 (s, 3H), 3.20 (s, 3H), 5.39 (s, 2H), 7.38-7.46 (m, 2H), 7.88-7.95 (m, 2H), 7.97 (br. s., 1H), 8.10-8.28 (m, 2H), 8.34-8.41 (m, 2H), 10.21 (s, 1H).

### Example 232

### N⁴-[1-(2-cyanobenzyl)-3,5-dimethyl-1H-pyrazol-4-yl]quinoline-2,4-dicarboxamide

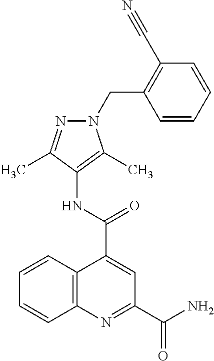 [SMILES: Cc1nn(Cc2ccccc2C#N)c(C)c1NC(=O)c1cc(C(N)=O)nc2ccccc12]

In analogy to example 118), 126 mg (0.56 mmol) 2-[(4-amino-3,5-dimethyl-1H-pyrazol-1-yl)methyl]benzonitrile (intermediate 10C) and 100 mg (0.46 mmol) 2-carbamoylquinoline-4-carboxylic acid (intermediate 4A) were reacted to give after purification via preparative HPLC (method 3) 118 mg (55%) of the desired title compound.

¹H-NMR (300 MHz, DMSO d₆) δ (ppm)=2.13 (s, 3H), 2.26 (s, 3H), 5.44 (s, 2H), 7.10 (d, 1H), 7.47-7.56 (m, 1H), 7.71 (td, 1H), 7.77-7.86 (m, 1H), 7.86-7.97 (m, 3H), 8.17-8.30 (m, 3H), 8.37 (br. s., 1H), 10.10 (s, 1H).

### Example 233

### 2-cyclopropyl-N-[1-(4-fluorobenzyl)-3,5-dimethyl-1H-pyrazol-4-yl]-6-methoxyquinoline-4-carboxamide

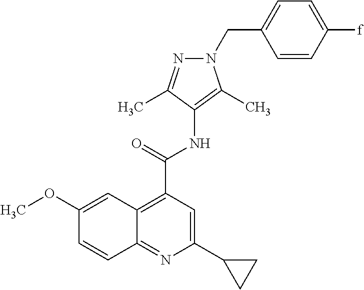

In analogy to example 118), 108 mg (0.49 mmol) 1-(4-fluorobenzyl)-3,5-dimethyl-1H-pyrazol-4-amine (intermediate 1C) and 100 mg (0.41 mmol) 2-cyclopropyl-6-methoxyquinoline-4-carboxylic acid were reacted to give after purification via preparative HPLC (method 4) 127 mg (64%) of the desired title compound.

¹H-NMR (300 MHz, DMSO d₆) δ (ppm)=1.00-1.13 (m, 4H), 2.13 (s, 3H), 2.17 (s, 3H), 2.27-2.38 (m, 1H), 3.80-3.83 (m, 3H), 5.23 (s, 2H), 7.14-7.28 (m, 4H), 7.35-7.46 (m, 2H), 7.57 (s, 1H), 7.82 (d, 1H), 9.85 (s, 1H).

### Example 234

### 6-bromo-N-{1-[(3-cyanopyridin-2-yl)methyl]-3,5-dimethyl-1H-pyrazol-4-yl}-2-(trifluoromethyl)quinoline-4-carboxamide

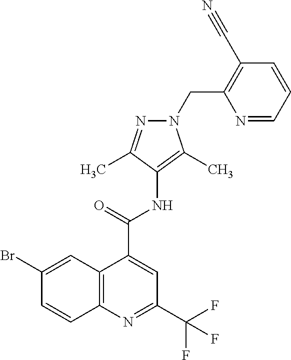 [SMILES: Cc1nn(Cc2ncccc2C#N)c(C)c1NC(=O)c1cc(C(F)(F)F)nc2ccc(Br)cc12]

In analogy to example 118), 100 mg (0.44 mmol) 2-[(4-amino-3,5-dimethyl-1H-pyrazol-1-yl)methyl]nicotinonitrile (intermediate 40C) and 117 mg (0.37 mmol) 6-bromo-2-(trifluoromethyl)quinoline-4-carboxylic acid (intermediate 1A) were reacted to give after purification via preparative HPLC (method 3) 7.7 mg (3.8%) of the desired title compound.

¹H-NMR (400 MHz, DMSO d₆) δ (ppm)=2.08 (s, 3H), 2.32 (s, 3H), 5.53 (s, 2H), 7.57 (dd, 1H), 8.12-8.17 (m, 1H), 8.23 (d, 1H), 8.29 (s, 1H), 8.37 (dd, 1H), 8.50 (d, 1H), 8.79 (dd, 1H), 10.18 (s, 1H).

### Example 235

### N⁴-[1-(2-cyanobenzyl)-3,5-dimethyl-1H-pyrazol-4-yl]-6,7-difluoroquinoline-2,4-dicarboxamide

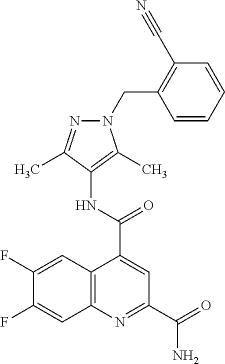 [SMILES: Cc1nn(Cc2ccccc2C#N)c(C)c1NC(=O)c1cc(C(N)=O)nc2cc(F)c(F)cc12]

In analogy to example 118), 108 mg (0.48 mmol) 2-[(4-amino-3,5-dimethyl-1H-pyrazol-1-yl)methyl]benzonitrile (intermediate 10C) and 100 mg (0.40 mmol) 2-carbamoyl-6,7-difluoroquinoline-4-carboxylic acid (intermediate 3A) were reacted to give after purification via preparative HPLC (method 3) 35 mg (18%) of the desired title compound.

¹H-NMR (300 MHz, DMSO d₆) δ (ppm)=2.11 (s, 3H), 2.24 (s, 3H), 5.43 (s, 2H), 7.10 (d, 1H), 7.48-7.56 (m, 1H), 7.67-7.75 (m, 1H), 7.90 (dd, 1H), 7.97 (s, 1H), 8.11-8.30 (m, 2H), 8.36-8.43 (m, 2H), 10.24 (s, 1H).

### Example 236

### 6,7-difluoro-N⁴-[1-(3-fluoro-4-methoxybenzyl)-3,5-dimethyl-1H-pyrazol-4-yl]quinoline-2,4-dicarboxamide

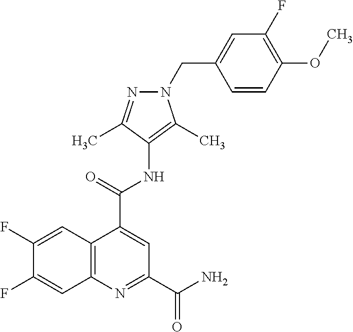 [SMILES: COc1ccc(Cn2nc(C)c(NC(=O)c3cc(C(N)=O)nc4cc(F)c(F)cc34)c2C)cc1F]

In analogy to example 118), 118 mg (0.48 mmol) 1-(3-fluoro-4-methoxybenzyl)-3,5-dimethyl-1H-pyrazol-4-amine (intermediate 42C) and 100 mg (0.40 mmol) 2-carbamoyl-6,7-difluoroquinoline-4-carboxylic acid (intermediate 3A) were reacted to give after purification via preparative HPLC (method 3) 80 mg (38%) of the desired title compound.

¹H-NMR (300 MHz, DMSO d₆) δ (ppm)=2.12 (s, 3H), 2.16 (s, 3H), 3.81 (s, 3H), 5.18 (s, 2H), 6.94-7.07 (m, 2H), 7.14 (t, 1H), 7.97 (br. s., 1H), 8.11-8.30 (m, 2H), 8.32-8.42 (m, 2H), 10.17 (s, 1H).

### Example 237

### 6-chloro-7-fluoro-N⁴-[1-(3-fluoro-4-methoxybenzyl)-3,5-dimethyl-1H-pyrazol-4-yl]quinoline-2,4-dicarboxamide

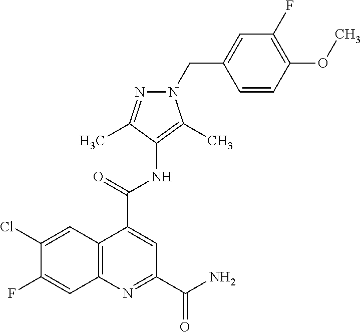 [SMILES: COc1ccc(Cn2nc(C)c(NC(=O)c3cc(C(N)=O)nc4cc(F)c(Cl)cc34)c2C)cc1F]

In analogy to example 118), 111 mg (0.45 mmol) 1-(3-fluoro-4-methoxybenzyl)-3,5-dimethyl-1H-pyrazol-4-amine (intermediate 42C) and 100 mg (0.37 mmol) 2-carbamoyl-6-chloro-7-fluoroquinoline-4-carboxylic acid (intermediate 32A) were reacted to give after purification via preparative HPLC (method 3) 24 mg (12%) of the desired title compound.

¹H-NMR (300 MHz, DMSO d₆) δ (ppm)=2.16 (s, 3H), 2.12 (s, 3H), 3.81 (s, 3H), 5.18 (s, 2H), 6.95-7.07 (m, 2H), 7.15 (t, 1H), 7.99 (br. s., 1H), 8.12 (d, 1H), 8.34-8.42 (m, 2H), 8.51 (d, 1H), 10.20 (s, 1H).

### Example 238

### N⁴-[1-(3-fluoro-4-methoxybenzyl)-3,5-dimethyl-1H-pyrazol-4-yl]quinoline-2,4-dicarboxamide

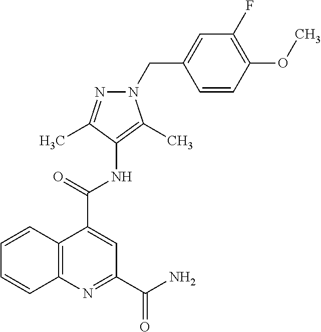 [SMILES: COc1ccc(Cn2nc(C)c(NC(=O)c3cc(C(N)=O)nc4ccccc34)c2C)cc1F]

In analogy to example 118), 138 mg (0.45 mmol) 1-(3-fluoro-4-methoxybenzyl)-3,5-dimethyl-1H-pyrazol-4-amine (intermediate 42C) and 100 mg (0.46 mmol) 2-carbamoylquinoline-4-carboxylic acid (intermediate 4A) were reacted to give after purification via preparative HPLC (method 3) 91 mg (43%) of the desired title compound.

¹H-NMR (300 MHz, DMSO d₆) δ (ppm)=2.14 (s, 3H), 2.18 (s, 3H), 3.81 (s, 3H), 5.18 (s, 2H), 6.95-7.07 (m, 2H), 7.15 (t, 1H), 7.77-7.85 (m, 1H), 7.87-7.97 (m, 2H), 8.18-8.30 (m, 3H), 8.39 (br. s., 1H), 10.05 (s, 1H).

### Example 239

### N-[1-(3-fluoro-4-methoxybenzyl)-3,5-dimethyl-1H-pyrazol-4-yl]-2-methoxyquinoline-4-carboxamide

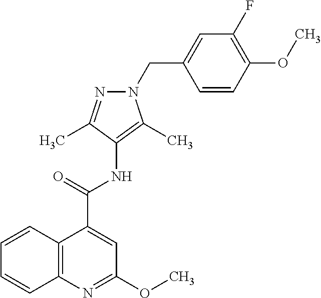 [SMILES: COc1cc(C(=O)Nc2c(C)nn(Cc3ccc(OC)c(F)c3)c2C)c2ccccc2n1]

In analogy to example 118), 147 mg (0.59 mmol) 1-(3-fluoro-4-methoxybenzyl)-3,5-dimethyl-1H-pyrazol-4-amine (intermediate 42C) and 100 mg (0.49 mmol) 2-methoxyquinoline-4-carboxylic acid were reacted to give after purification via preparative HPLC (method 3) 142 mg (65%) of the desired title compound.

¹H-NMR (300 MHz, DMSO d₆) δ (ppm)=2.12 (s, 3H), 2.16 (s, 3H), 3.80 (s, 3H), 4.03 (s, 3H), 5.17 (s, 2H), 6.94-7.05 (m, 2H), 7.14 (t, 1H), 7.21 (s, 1H), 7.44-7.55 (m, 1H), 7.72 (td, 1H), 7.82-7.89 (m, 1H), 8.04 (d, 1H), 9.88 (s, 1H).

### Example 240

### N⁴-[1-(4-cyanobenzyl)-5-methyl-3-(trifluoromethyl)-1H-pyrazol-4-yl]-7-fluoroquinoline-2,4-dicarboxamide

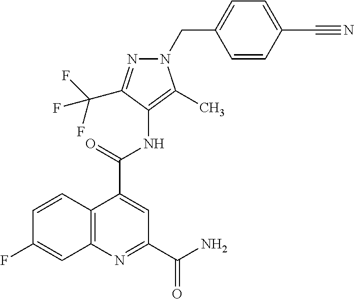 [SMILES: Cc1c(NC(=O)c2cc(C(N)=O)nc3cc(F)ccc23)c(C(F)(F)F)nn1Cc1ccc(C#N)cc1]

In analogy to example 118), 144 mg (0.51 mmol) 4-{[4-amino-5-methyl-3-(trifluoromethyl)-1H-pyrazol-1-yl]methyl}benzonitrile (intermediate 26C) and 100 mg (0.43 mmol) 2-carbamoyl-7-fluoroquinoline-4-carboxylic acid (intermediate 37A) were reacted to give after purification via preparative HPLC (method 3) 98 mg (44%) of the desired title compound.

¹H-NMR (300 MHz, DMSO d₆) δ (ppm)=2.27 (s, 3H), 5.61 (s, 2H), 7.38 (d, 2H), 7.73-7.84 (m, 1H), 7.86-7.95 (m, 3H), 7.97 (br. s., 1H), 8.23-8.33 (m, 2H), 8.40 (br. s., 1H), 10.48 (s, 1H).

### Example 241

### 6-chloro-N⁴-[1-(4-cyanobenzyl)-5-methyl-3-(trifluoromethyl)-1H-pyrazol-4-yl]-8-fluoroquinoline-2,4-dicarboxamide

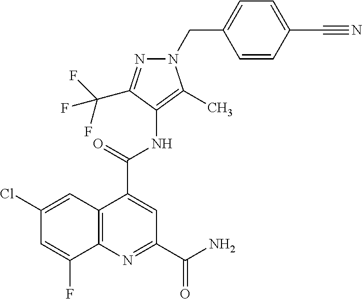 [SMILES: Cc1c(NC(=O)c2cc(C(N)=O)nc3c(F)cc(Cl)cc23)c(C(F)(F)F)nn1Cc1ccc(C#N)cc1]

In analogy to example 118), 125 mg (0.45 mmol) 4-{[4-amino-5-methyl-3-(trifluoromethyl)-1H-pyrazol-1-yl]methyl}benzonitrile (intermediate 26C) and 100 mg (0.37 mmol) 2-carbamoyl-6-chloro-8-fluoroquinoline-4-carboxylic acid (intermediate 49A) were reacted to give after purification via preparative HPLC (method 3) 35 mg (16%) of the desired title compound.

¹H-NMR (300 MHz, DMSO d₆) δ (ppm)=2.26 (s, 3H), 5.60 (s, 2H), 7.38 (d, 2H), 7.89 (d, 2H), 8.00-8.10 (m, 3H), 8.29 (br. s., 1H), 8.42 (s, 1H), 10.56 (s, 1H).

### Example 242

### N⁴-[1-(4-cyanobenzyl)-5-methyl-3-(trifluoromethyl)-1H-pyrazol-4-yl]-7-fluoro-6-methoxyquinoline-2,4-dicarboxamide

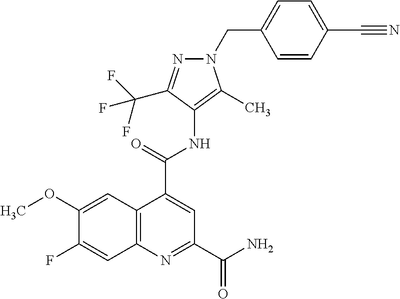 [SMILES: COc1cc2c(C(=O)Nc3c(C(F)(F)F)nn(Cc4ccc(C#N)cc4)c3C)cc(C(N)=O)nc2cc1F]

In analogy to example 118), 76 mg (0.27 mmol) 4-{[4-amino-5-methyl-3-(trifluoromethyl)-1H-pyrazol-1-yl]methyl}benzonitrile (intermediate 26C) and 100 mg (0.23 mmol, about 60% purity) 2-carbamoyl-7-fluoro-6-methoxyquinoline-4-carboxylic acid (intermediate 45A) were reacted to give after purification via preparative HPLC (method 3) 19 mg (15%) of the desired title compound.

¹H-NMR (400 MHz, DMSO d₆) δ (ppm)=2.27 (s, 3H), 3.96 (s, 3H), 5.59 (s, 2H), 7.40 (d, 2H), 7.69 (d, 1H), 7.84-7.97 (m, 4H), 8.24 (s, 1H), 8.27-8.30 (m, 1H), 10.43 (s, 1H).

### Example 243

### N⁴-[1-(4-cyanobenzyl)-5-methyl-3-(trifluoromethyl)-1H-pyrazol-4-yl]-5,7-difluoroquinoline-2,4-dicarboxamide

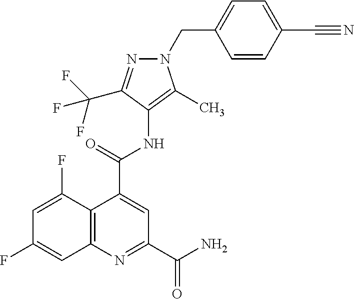 [SMILES: Cc1c(NC(=O)c2cc(C(N)=O)nc3cc(F)cc(F)c23)c(C(F)(F)F)nn1Cc1ccc(C#N)cc1]

In analogy to example 118), 106 mg (0.38 mmol) 4-{[4-amino-5-methyl-3-(trifluoromethyl)-1H-pyrazol-1-yl]methyl}benzonitrile (intermediate 26C) and 130 mg (0.31 mmol, about 60% purity) 2-carbamoyl-5,7-difluoroquinoline-4-carboxylic acid (intermediate 44A) were reacted to give after purification via preparative HPLC (method 5d) 18 mg (11%) of the desired title compound.

¹H-NMR (400 MHz, DMSO d₆) δ (ppm)=2.26 (s, 3H), 5.59 (s, 2H), 7.37 (d, 2H), 7.80-7.91 (m, 4H), 8.01 (s, 1H), 8.04 (s, 1H), 8.40 (s, 1H), 10.29 (s, 1H).

### Example 244

### N⁴-[1-(4-cyanobenzyl)-5-methyl-3-(trifluoromethyl)-1H-pyrazol-4-yl]-5-methylquinoline-2,4-dicarboxamide

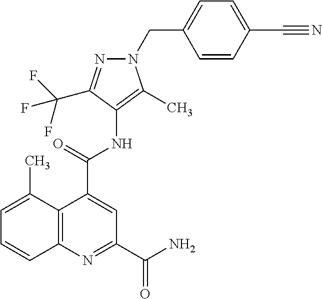 [SMILES: Cc1cccc2nc(C(N)=O)cc(C(=O)Nc3c(C(F)(F)F)nn(Cc4ccc(C#N)cc4)c3C)c12]

In analogy to example 118), 58 mg (0.21 mmol) 4-{[4-amino-5-methyl-3-(trifluoromethyl)-1H-pyrazol-1-yl]methyl}benzonitrile (intermediate 26C) and 60 mg (0.26 mmol) 2-carbamoyl-5-methylquinoline-4-carboxylic acid (intermediate 40A) were reacted to give after purification via preparative HPLC (method 4) 16 mg (11%) of the desired title compound.

¹H-NMR (400 MHz, DMSO d₆) δ (ppm)=2.30 (s, 3H), 3.33 (s, 3H), 5.62 (s, 2H), 7.41 (d, 2H), 7.64 (d, 1H), 7.83 (dd, 1H), 7.88-7.93 (m, 3H), 8.06-8.11 (m, 2H), 8.36-8.42 (m, 1H), 10.47 (s, 1H).

### Example 245

### N⁴-[1-(4-cyanobenzyl)-5-methyl-3-(trifluoromethyl)-1H-pyrazol-4-yl]-6-methoxyquinoline-2,4-dicarboxamide

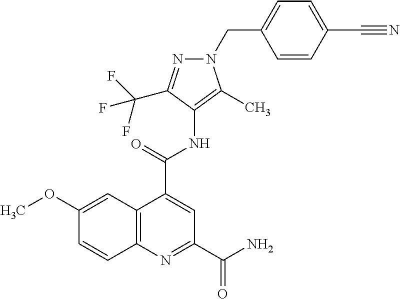 [SMILES: COc1ccc2nc(C(N)=O)cc(C(=O)Nc3c(C(F)(F)F)nn(Cc4ccc(C#N)cc4)c3C)c2c1]

In analogy to example 118), 130 mg (0.46 mmol) 4-{[4-amino-5-methyl-3-(trifluoromethyl)-1H-pyrazol-1-yl]methyl}benzonitrile (intermediate 26C) and 100 mg (0.26 mmol) 2-carbamoyl-6-methoxyquinoline-4-carboxylic acid (intermediate 46A) were reacted to give after purification via preparative HPLC (method 3) 95 mg (47%) of the desired title compound.

¹H-NMR (300 MHz, DMSO d₆) δ (ppm)=2.27 (s, 3H), 3.88 (s, 3H), 5.60 (s, 2H), 7.39 (d, 2H), 7.51 (d, 1H), 7.60 (dd, 1H), 7.83 (br. s., 1H), 7.89 (d, 2H), 8.11 (d, 1H), 8.22 (s, 1H), 8.32 (br. s., 1H), 10.40 (s, 1H).

### Example 246

### 7-chloro-N⁴-[1-(4-cyanobenzyl)-5-methyl-3-(trifluoromethyl)-1H-pyrazol-4-yl]quinoline-2,4-dicarboxamide

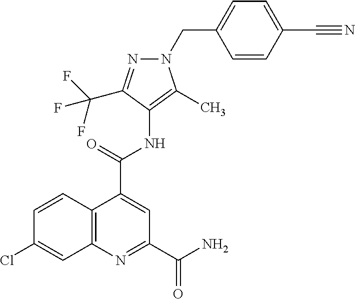 [SMILES: Cc1c(NC(=O)c2cc(C(N)=O)nc3cc(Cl)ccc23)c(C(F)(F)F)nn1Cc1ccc(C#N)cc1]

In analogy to example 118), 112 mg (0.40 mmol) 4-{[4-amino-5-methyl-3-(trifluoromethyl)-1H-pyrazol-1-yl]methyl}benzonitrile (intermediate 26C) and 100 mg (0.40 mmol) 2-carbamoyl-7-chloroquinoline-4-carboxylic acid (intermediate 48A) were reacted to give after purification via a Biotage chromatography system (10 g snap KP-Sil column, hexane/80-100% ethyl acetate, then ethyl acetate/0-30% methanol) 88 mg (40%) of the desired title compound.

¹H-NMR (300 MHz, DMSO d₆) δ (ppm)=2.26 (s, 3H), 5.60 (s, 2H), 7.38 (d, 2H), 7.80-7.93 (m, 3H), 7.98 (s, 1H), 8.16-8.26 (m, 2H), 8.30 (s, 1H), 8.40 (s, 1H), 10.49 (s, 1H).

### Example 247

### N⁴-[1-(4-cyanobenzyl)-5-methyl-3-(trifluoromethyl)-1H-pyrazol-4-yl]-8-fluoroquinoline-2,4-dicarboxamide

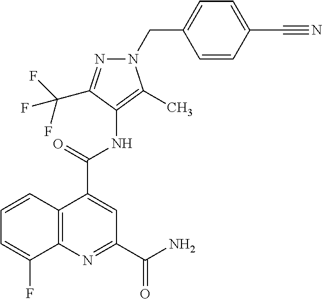 [SMILES: Cc1c(NC(=O)c2cc(C(N)=O)nc3c(F)cccc23)c(C(F)(F)F)nn1Cc1ccc(C#N)cc1]

In analogy to example 118), 108 mg (0.38 mmol) 4-{[4-amino-5-methyl-3-(trifluoromethyl)-1H-pyrazol-1-yl]methyl}benzonitrile (intermediate 26C) and 90 mg (0.38 mmol) 2-carbamoyl-8-fluoroquinoline-4-carboxylic acid (intermediate 47A) were reacted to give after purification via preparative HPLC (method 3) 64 mg (31%) of the desired title compound.

¹H-NMR (400 MHz, DMSO d₆) δ (ppm)=2.27 (s, 3H), 5.60 (s, 2H), 7.38 (d, 2H), 7.76-7.85 (m, 2H), 7.86-7.92 (m, 2H), 7.97-8.04 (m, 2H), 8.26 (s, 1H), 8.33 (s, 1H), 10.49 (s, 1H).

### Example 248

### 6-chloro-N⁴-[1-(4-cyanobenzyl)-5-methyl-3-(trifluoromethyl)-1H-pyrazol-4-yl]quinoline-2,4-dicarboxamide

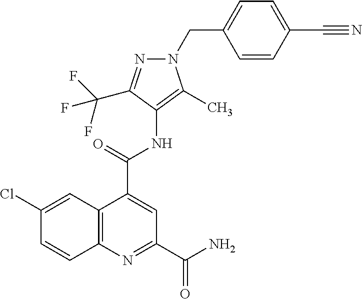 [SMILES: Cc1c(NC(=O)c2cc(C(N)=O)nc3ccc(Cl)cc23)c(C(F)(F)F)nn1Cc1ccc(C#N)cc1]

In analogy to example 118), 268 mg (0.96 mmol) 4-{[4-amino-5-methyl-3-(trifluoromethyl)-1H-pyrazol-1-yl]methyl}benzonitrile (intermediate 26C) and 200 mg (0.80 mmol) 2-carbamoyl-6-chloroquinoline-4-carboxylic acid (intermediate 38A) were reacted to give after purification via preparative HPLC (method 4) 91 mg (22%) of the desired title compound.

¹H-NMR (400 MHz, DMSO d₆) δ (ppm)=2.26 (s, 3H), 5.60 (s, 2H), 7.39 (d, 2H), 7.89 (d, 2H), 7.94 (s, 1H), 7.98 (dd, 1H), 8.20-8.25 (m, 2H), 8.35 (s, 1H), 8.41 (s, 1H), 10.49 (s, 1H).

### Example 249

### N⁴-[1-(4-cyanobenzyl)-5-methyl-3-(trifluoromethyl)-1H-pyrazol-4-yl]-5-fluoroquinoline-2,4-dicarboxamide

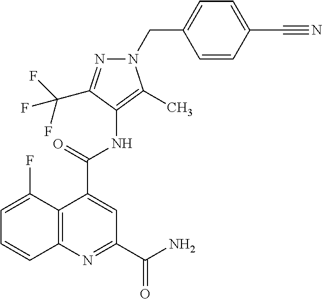 [SMILES: Cc1c(NC(=O)c2cc(C(N)=O)nc3cccc(F)c23)c(C(F)(F)F)nn1Cc1ccc(C#N)cc1]

In analogy to example 118), 120 mg (0.43 mmol) 4-{[4-amino-5-methyl-3-(trifluoromethyl)-1H-pyrazol-1-yl]methyl}benzonitrile (intermediate 26C) and 100 mg (0.43 mmol) 2-carbamoyl-5-fluoroquinoline-4-carboxylic acid (intermediate 39A) were reacted to give after purification via a Biotage chromatography system (10 g snap KP-Sil column, hexane/80-100% ethyl acetate, then ethyl acetate/0-50% methanol) 110 mg (48%) of the desired title compound.

¹H-NMR (300 MHz, DMSO d₆) δ (ppm)=2.26 (s, 3H), 5.60 (s, 2H), 7.38 (d, 2H), 7.74-7.84 (m, 1H), 7.84-7.92 (m, 3H), 7.97 (br. s., 1H), 8.24-8.32 (m, 2H), 8.39 (s, 1H), 10.48 (s, 1H).

### Example 250

### N⁴-[1-(4-cyanobenzyl)-5-methyl-3-(trifluoromethyl)-1H-pyrazol-4-yl]-5-fluoroquinoline-2,4-dicarboxamide

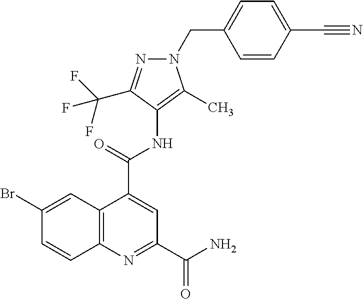 [SMILES: Cc1c(NC(=O)c2cc(C(N)=O)nc3ccc(Br)cc23)c(C(F)(F)F)nn1Cc1ccc(C#N)cc1]

In analogy to example 118), 617 mg (2.20 mmol) 4-{[4-amino-5-methyl-3-(trifluoromethyl)-1H-pyrazol-1-yl]methyl}benzonitrile (intermediate 26C) and 570 mg (1.84 mmol) 6-bromo-2-carbamoylquinoline-4-carboxylic acid (intermediate 2A) were reacted to give after two subsequent purifications via a Biotage chromatography system (two times 25 g snap KP-Sil column, hexane/0-100% ethyl acetate, then ethyl acetate/0-60% methanol) 480 mg (45%) of the desired title compound.

¹H-NMR (400 MHz, DMSO d₆) δ (ppm)=2.26 (s, 3H), 5.60 (s, 2H), 7.39 (d, 2H), 7.89 (d, 2H), 7.94 (s, 1H), 8.08 (dd, 1H), 8.14 (d, 1H), 8.34 (s, 1H), 8.38-8.44 (m, 2H), 10.49 (s, 1H).

### Example 251

### N⁴-[1-(4-cyanobenzyl)-3,5-dimethyl-1H-pyrazol-4-yl]-7-fluoroquinoline-2,4-dicarboxamide

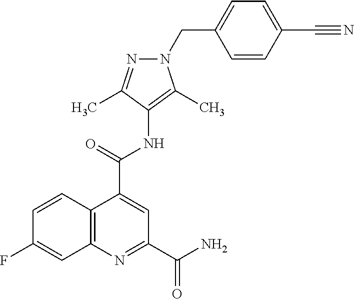 [SMILES: Cc1nn(Cc2ccc(C#N)cc2)c(C)c1NC(=O)c1cc(C(N)=O)nc2cc(F)ccc12]

In analogy to example 118), 116 mg (0.51 mmol) 4-[(4-amino-3,5-dimethyl-1H-pyrazol-1-yl)methyl]benzonitrile (intermediate 8C) and 100 mg (0.40 mmol) 2-carbamoyl-7-fluoroquinoline-4-carboxylic acid (intermediate 37A) were reacted to give after purification via preparative HPLC (method 3) 103 mg (53%) of the desired title compound.

¹H-NMR (300 MHz, DMSO d₆) δ (ppm)=2.14 (s, 3H), 2.17 (s, 3H), 5.37 (s, 2H), 7.31 (d, 2H), 7.77 (ddd, 1H), 7.84 (d, 2H), 7.91 (dd, 1H), 7.95 (br. s., 1H), 8.28 (s, 1H), 8.31-8.42 (m, 2H), 10.14 (s, 1H).

### Example 252

### 6-chloro-N⁴-[1-(4-cyanobenzyl)-3,5-dimethyl-1H-pyrazol-4-yl]-8-fluoroquinoline-2,4-dicarboxamide

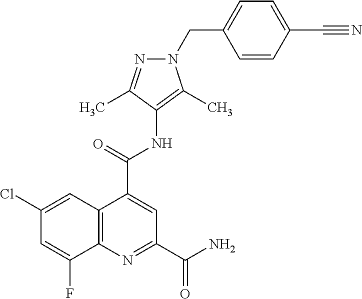 [SMILES: Cc1nn(Cc2ccc(C#N)cc2)c(C)c1NC(=O)c1cc(C(N)=O)nc2c(F)cc(Cl)cc12]

In analogy to example 118), 101 mg (0.45 mmol) 4-[(4-amino-3,5-dimethyl-1H-pyrazol-1-yl)methyl]benzonitrile (intermediate 8C) and 100 mg (0.37 mmol) 2-carbamoyl-6-chloro-8-fluoroquinoline-4-carboxylic acid (intermediate 49A) were reacted to give after purification via preparative HPLC (method 3) 85 mg (44%) of the desired title compound.

¹H-NMR (400 MHz, DMSO d₆) δ (ppm)=2.16 (s, 3H), 2.13 (s, 3H), 5.37 (s, 2H), 7.32 (d, 2H), 7.82-7.87 (m, 2H), 7.96-8.05 (m, 2H), 8.14 (d, 1H), 8.25 (s, 1H), 8.45 (s, 1H), 10.22 (s, 1H).

### Example 253

### N⁴-[1-(4-cyanobenzyl)-3,5-dimethyl-1H-pyrazol-4-yl]-5,7-difluoroquinoline-2,4-dicarboxamide

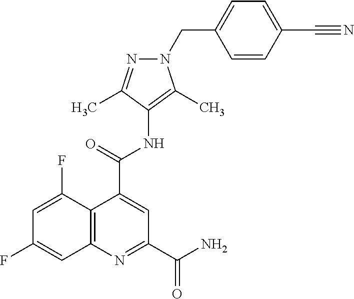 [SMILES: Cc1nn(Cc2ccc(C#N)cc2)c(C)c1NC(=O)c1cc(C(N)=O)nc2cc(F)cc(F)c12]

In analogy to example 118), 162 mg (0.71 mmol) 4-[(4-amino-3,5-dimethyl-1H-pyrazol-1-yl)methyl]benzonitrile (intermediate 8C) and 150 mg (0.37 mmol) 2-carbamoyl-5,7-difluoroquinoline-4-carboxylic acid (intermediate 44A) were reacted to give after purification via preparative HPLC (method 5c) 165 mg (57%) of the desired title compound.

¹H-NMR (400 MHz, DMSO d₆) δ (ppm)=2.17 (s, 3H), 2.13 (s, 3H), 5.36 (s, 2H), 7.31 (d, 2H), 7.75-7.90 (m, 4H), 8.00 (s, 1H), 8.10 (s, 1H), 8.39 (s, 1H), 9.90 (s, 1H).

### Example 254

### N⁴-[1-(4-cyanobenzyl)-3,5-dimethyl-1H-pyrazol-4-yl]-7-fluoro-6-methoxyquinoline-2,4-dicarboxamide

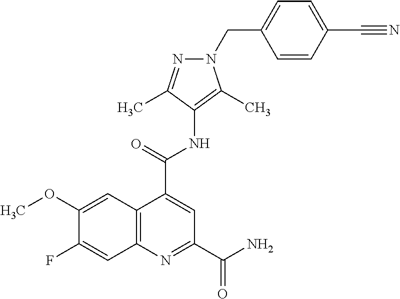 [SMILES: COc1cc2c(C(=O)Nc3c(C)nn(Cc4ccc(C#N)cc4)c3C)cc(C(N)=O)nc2cc1F]

In analogy to example 118), 62 mg (0.27 mmol) 4-[(4-amino-3,5-dimethyl-1H-pyrazol-1-yl)methyl]benzonitrile (intermediate 8C) and 100 mg (0.23 mmol, about 60% purity) 2-carbamoyl-7-fluoro-6-methoxyquinoline-4-carboxylic acid (intermediate 45A) were reacted to give after purification via preparative HPLC (method 3) 26 mg (24%) of the desired title compound.

¹H-NMR (400 MHz, DMSO d₆) δ (ppm)=2.15 (s, 3H), 2.17 (s, 3H), 3.97 (s, 3H), 5.37 (s, 2H), 7.33 (d, 2H), 7.78 (d, 1H), 7.82-7.86 (m, 3H), 7.93 (d, 1H), 8.25-8.29 (m, 2H), 10.11 (s, 1H).

### Example 255

### 8-chloro-N⁴-[1-(4-cyanobenzyl)-3,5-dimethyl-1H-pyrazol-4-yl]quinoline-2,4-dicarboxamide

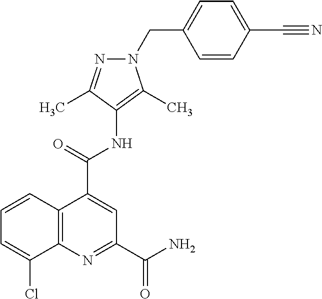 [SMILES: Cc1nn(Cc2ccc(C#N)cc2)c(C)c1NC(=O)c1cc(C(N)=O)nc2c(Cl)cccc12]

In analogy to example 118), 108 mg (0.48 mmol) 4-[(4-amino-3,5-dimethyl-1H-pyrazol-1-yl)methyl]benzonitrile (intermediate 8C) and 100 mg (0.40 mmol) 2-carbamoyl-8-chloroquinoline-4-carboxylic acid (intermediate 43A) were reacted to give after purification via preparative HPLC (method 4) 6.7 mg (3.3%) of the desired title compound.

¹H-NMR (400 MHz, DMSO d₆) δ (ppm)=2.16 (s, 3H), 2.19 (s, 3H), 5.39 (s, 2H), 7.33 (d, 2H), 7.80 (dd, 1H), 7.86 (d, 2H), 8.08 (d, 1H), 8.12-8.17 (m, 2H), 8.23 (dd, 1H), 8.37 (s, 1H), 10.17 (s, 1H).

### Example 256

### N⁴-[1-(4-cyanobenzyl)-3,5-dimethyl-1H-pyrazol-4-yl]-7-fluoro-6-methylquinoline-2,4-dicarboxamide

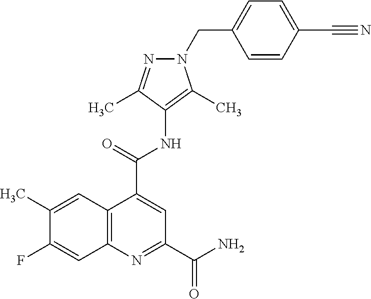 [SMILES: Cc1cc2c(C(=O)Nc3c(C)nn(Cc4ccc(C#N)cc4)c3C)cc(C(N)=O)nc2cc1F]

In analogy to example 118), 104 mg (0.48 mmol) 4-[(4-amino-3,5-dimethyl-1H-pyrazol-1-yl)methyl]benzonitrile (intermediate 8C) and 100 mg (0.40 mmol) 2-carbamoyl-7-fluoro-6-methylquinoline-4-carboxylic acid (intermediate 50A) were reacted to give after purification via preparative HPLC (method 4) 93 mg (49%) of the desired title compound.

¹H-NMR (400 MHz, DMSO d₆) δ (ppm)=2.16 (s, 3H), 2.18 (s, 3H), 2.50 (s, 3H), 5.39 (s, 2H), 7.34 (d, 2H), 7.80-7.90 (m, 3H), 7.92 (s, 1H), 8.18 (d, 1H), 8.26 (s, 1H), 8.35 (s, 1H), 10.13 (s, 1H).

### Example 257

### N⁴-[1-(4-cyanobenzyl)-3,5-dimethyl-1H-pyrazol-4-yl]-5-methylquinoline-2,4-dicarboxamide

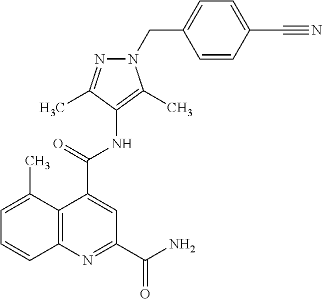 [SMILES: Cc1nn(Cc2ccc(C#N)cc2)c(C)c1NC(=O)c1cc(C(N)=O)nc2cccc(C)c12]

In analogy to example 118), 47 mg (0.21 mmol) 4-[(4-amino-3,5-dimethyl-1H-pyrazol-1-yl)methyl]benzonitrile (intermediate 8C) and 60 mg (0.40 mmol) 2-carbamoyl-5-methylquinoline-4-carboxylic acid (intermediate 40A) were reacted to give after purification via preparative HPLC (method 4) 44 mg (37%) of the desired title compound.

¹H-NMR (400 MHz, DMSO d₆) δ (ppm)=2.17 (s, 3H), 2.20 (s, 3H), 2.51 (s, 3H), 5.38 (s, 2H), 7.34 (d, 2H), 7.63 (d, 1H), 7.79-7.87 (m, 3H), 7.90 (d, 1H), 8.07-8.11 (m, 2H), 8.34-8.43 (m, 1H), 10.10 (s, 1H).

### Example 258

### N⁴-[1-(4-cyanobenzyl)-3,5-dimethyl-1H-pyrazol-4-yl]-6-methoxyquinoline-2,4-dicarboxamide

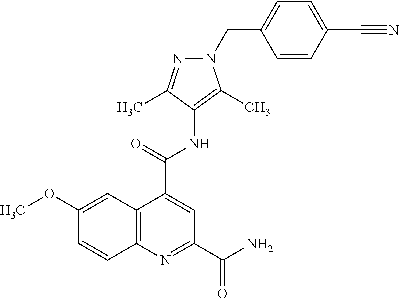 [SMILES: COc1ccc2nc(C(N)=O)cc(C(=O)Nc3c(C)nn(Cc4ccc(C#N)cc4)c3C)c2c1]

In analogy to example 118), 105 mg (0.46 mmol) 4-[(4-amino-3,5-dimethyl-1H-pyrazol-1-yl)methyl]benzonitrile (intermediate 8C) and 100 mg (0.39 mmol) 2-carbamoyl-6-methoxyquinoline-4-carboxylic acid (intermediate 46A) were reacted to give after purification via preparative HPLC (method 3) 120 mg (66%) of the desired title compound.

¹H-NMR (300 MHz, DMSO d₆) δ (ppm)=2.15 (s, 3H), 2.17 (s, 3H), 3.89 (s, 3H), 5.37 (s, 2H), 7.33 (d, 2H), 7.55-7.63 (m, 2H), 7.84 (d, 3H), 8.10 (d, 1H), 8.25 (s, 1H), 8.30 (d, 1H), 10.07 (s, 1H).

### Example 259

### N⁴-[1-(4-cyanobenzyl)-3,5-dimethyl-1H-pyrazol-4-yl]quinoline-2,4-dicarboxamide

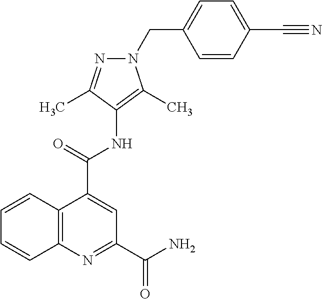 [SMILES: Cc1nn(Cc2ccc(C#N)cc2)c(C)c1NC(=O)c1cc(C(N)=O)nc2ccccc12]

In analogy to example 118), 105 mg (0.46 mmol) 4-[(4-amino-3,5-dimethyl-1H-pyrazol-1-yl)methyl]benzonitrile (intermediate 8C) and 100 mg (0.46 mmol) 2-carbamoylquinoline-4-carboxylic acid (intermediate 4A) were reacted to give after purification via preparative HPLC (method 5c) 51 mg (24%) of the desired title compound.

¹H-NMR (400 MHz, DMSO d₆) δ (ppm)=2.15 (s, 3H), 2.17 (s, 3H), 5.37 (s, 2H), 7.32 (d, 2H), 7.78-7.96 (m, 5H), 8.20 (d, 1H), 8.25 (d, 1H), 8.27 (s, 1H), 8.38 (s, 1H), 10.08 (s, 1H).

### Example 260

### 7-chloro-N⁴-[1-(4-cyanobenzyl)-3,5-dimethyl-1H-pyrazol-4-yl]quinoline-2,4-dicarboxamide

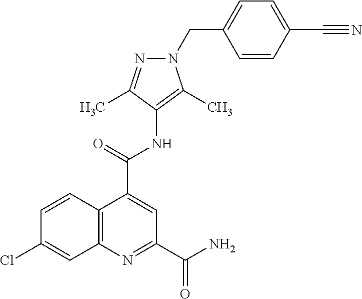 [SMILES: Cc1nn(Cc2ccc(C#N)cc2)c(C)c1NC(=O)c1cc(C(N)=O)nc2cc(Cl)ccc12]

In analogy to example 118), 90 mg (0.40 mmol) 4-[(4-amino-3,5-dimethyl-1H-pyrazol-1-yl)methyl]benzonitrile (intermediate 8C) and 100 mg (0.40 mmol) 2-carbamoyl-7-chloroquinoline-4-carboxylic acid (intermediate 48A) were reacted to give after purification via preparative HPLC (method 3) 41 mg (24%) of the desired title compound.

¹H-NMR (300 MHz, DMSO d₆) δ (ppm)=2.14 (s, 3H), 2.16 (s, 3H), 5.37 (s, 2H), 7.31 (d, 2H), 7.81-7.88 (m, 3H), 7.97 (s, 1H), 8.23 (d, 1H), 8.27-8.33 (m, 2H), 8.40 (s, 1H), 10.15 (s, 1H).

### Example 261

### 6-chloro-N⁴-[1-(4-cyanobenzyl)-3,5-dimethyl-1H-pyrazol-4-yl]quinoline-2,4-dicarboxamide

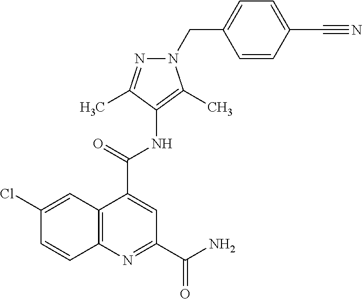 [SMILES: Cc1nn(Cc2ccc(C#N)cc2)c(C)c1NC(=O)c1cc(C(N)=O)nc2ccc(Cl)cc12]

In analogy to example 118), 217 mg (0.96 mmol) 4-[(4-amino-3,5-dimethyl-1H-pyrazol-1-yl)methyl]benzonitrile (intermediate 8C) and 200 mg (0.80 mmol) 2-carbamoyl-6-chloroquinoline-4-carboxylic acid (intermediate 38A) were reacted to give after purification via preparative HPLC (method 4) 33 mg (8.6%) of the desired title compound.

¹H-NMR (400 MHz, DMSO d₆) δ (ppm)=2.14 (s, 3H), 2.16 (s, 3H), 5.37 (s, 2H), 7.32 (d, 2H), 7.84 (d, 2H), 7.93 (s, 1H), 7.97 (dd, 1H), 8.22 (d, 1H), 8.31 (d, 1H), 8.38 (s, 1H), 8.40 (br. s., 1H), 10.17 (s, 1H).

### Example 262

### N⁴-[1-(4-cyanobenzyl)-3,5-dimethyl-1H-pyrazol-4-yl]-5-fluoroquinoline-2,4-dicarboxamide

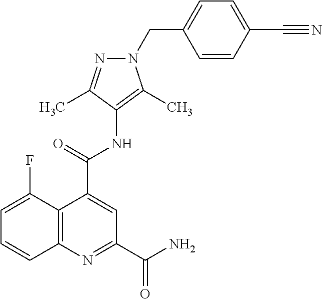 [SMILES: Cc1nn(Cc2ccc(C#N)cc2)c(C)c1NC(=O)c1cc(C(N)=O)nc2cccc(F)c12]

In analogy to example 118), 97 mg (0.43 mmol) 4-[(4-amino-3,5-dimethyl-1H-pyrazol-1-yl)methyl]benzonitrile (intermediate 8C) and 100 mg (0.43 mmol) 2-carbamoyl-5-fluoroquinoline-4-carboxylic acid (intermediate 39A) were reacted to give after purification via preparative HPLC (method 3) 97 mg (47%) of the desired title compound.

¹H-NMR (400 MHz, DMSO d₆) δ (ppm)=2.14 (s, 3H), 2.16 (s, 3H), 5.37 (s, 2H), 7.31 (d, 2H), 7.73-7.80 (m, 1H), 7.82-7.87 (m, 2H), 7.91 (dd, 1H), 7.96 (d, 1H), 8.27 (s, 1H), 8.34 (dd, 1H), 8.39 (d, 1H), 10.15 (s, 1H).

### Example 263

### N⁴-[1-(4-cyanobenzyl)-3,5-dimethyl-1H-pyrazol-4-yl]-8-fluoroquinoline-2,4-dicarboxamide

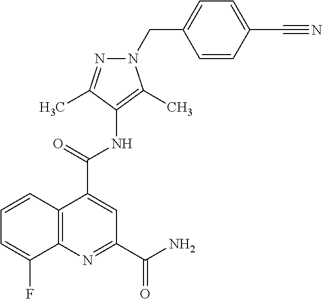 [SMILES: Cc1nn(Cc2ccc(C#N)cc2)c(C)c1NC(=O)c1cc(C(N)=O)nc2c(F)cccc12]

In analogy to example 118), 87 mg (0.38 mmol) 4-[(4-amino-3,5-dimethyl-1H-pyrazol-1-yl)methyl]benzonitrile (intermediate 8C) and 90 mg (0.38 mmol) 2-carbamoyl-8-fluoroquinoline-4-carboxylic acid (intermediate 47A) were reacted to give after purification via preparative HPLC (method 3) 77 mg (41%) of the desired title compound.

¹H-NMR (400 MHz, DMSO d₆) δ (ppm)=2.14 (s, 3H), 2.17 (s, 3H), 5.37 (s, 2H), 7.31 (d, 2H), 7.74-7.88 (m, 4H), 7.99 (s, 1H), 8.04-8.08 (m, 1H), 8.25 (s, 1H), 8.35 (s, 1H), 10.15 (s, 1H).

### Example 264

### N⁴-[1-(4-cyanobenzyl)-3,5-dimethyl-1H-pyrazol-4-yl]-6-methylquinoline-2,4-dicarboxamide

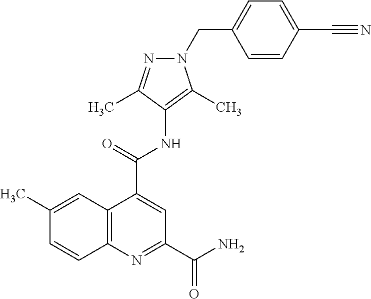 [SMILES: Cc1ccc2nc(C(N)=O)cc(C(=O)Nc3c(C)nn(Cc4ccc(C#N)cc4)c3C)c2c1]

In analogy to example 118), 98 mg (0.43 mmol) 4-[(4-amino-3,5-dimethyl-1H-pyrazol-1-yl)methyl]benzonitrile (intermediate 8C) and 100 mg (0.43 mmol) 2-carbamoyl-6-methylquinoline-4-carboxylic acid (intermediate 41A) were reacted to give after purification via preparative HPLC (method 3) 17 mg (8.5%) of the desired title compound.

¹H-NMR (300 MHz, DMSO d₆) δ (ppm)=2.15 (s, 3H), 2.17 (s, 3H), 2.54 (s, 3H), 5.37 (s, 2H), 7.33 (d, 2H), 7.77 (dd, 1H), 7.81-7.88 (m, 3H), 8.01 (s, 1H), 8.09 (d, 1H), 8.23 (s, 1H), 8.35 (s, 1H), 10.07 (s, 1H).

### Example 265

### N-[1-(4-cyanobenzyl)-3,5-dimethyl-1H-pyrazol-4-yl]-6-fluoro-2-methoxyquinoline-4-carboxamide

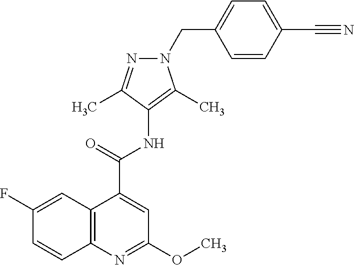 [SMILES: COc1cc(C(=O)Nc2c(C)nn(Cc3ccc(C#N)cc3)c2C)c2cc(F)ccc2n1]

In analogy to example 118), 86 mg (0.38 mmol) 4-[(4-amino-3,5-dimethyl-1H-pyrazol-1-yl)methyl]benzonitrile (intermediate 8C) and 70 mg (0.32 mmol) 6-fluoro-2-methoxyquinoline-4-carboxylic acid (intermediate 51A) were reacted to give after purification via preparative HPLC (method 4) 112 mg (78%) of the desired title compound.

¹H-NMR (300 MHz, DMSO d₆) δ (ppm)=2.12 (s, 3H), 2.14 (s, 3H), 4.03 (s, 3H), 5.36 (s, 2H), 7.30 (d, 2H), 7.34 (s, 1H), 7.61-7.69 (m, 1H), 7.78 (dd, 1H), 7.84 (d, 2H), 7.92 (dd, 1H), 9.98 (s, 1H).

### Example 266

### N⁴-[1-(2-cyanobenzyl)-3,5-dimethyl-1H-pyrazol-4-yl]-7-fluoroquinoline-2,4-dicarboxamide

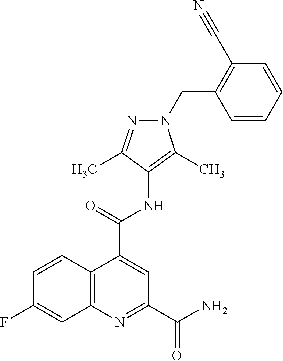 [SMILES: Cc1nn(Cc2ccccc2C#N)c(C)c1NC(=O)c1cc(C(N)=O)nc2cc(F)ccc12]

In analogy to example 118), 116 mg (0.51 mmol) 2-[(4-amino-3,5-dimethyl-1H-pyrazol-1-yl)methyl]benzonitrile (intermediate 10C) and 100 mg (0.43 mmol) 2-carbamoyl-7-fluoroquinoline-4-carboxylic acid (intermediate 37A) were reacted to give after purification via preparative HPLC (method 3) 86 mg (43%) of the desired title compound.

¹H-NMR (300 MHz, DMSO d₆) δ (ppm)=2.12 (s, 3H), 2.25 (s, 3H), 5.44 (s, 2H), 7.09 (d, 1H), 7.48-7.56 (m, 1H), 7.67-7.83 (m, 2H), 7.87-8.00 (m, 3H), 8.29 (s, 1H), 8.32-8.42 (m, 2H), 10.17 (s, 1H).

### Example 267

### N⁴-[1-(2-cyanobenzyl)-3,5-dimethyl-1H-pyrazol-4-yl]-5-fluoroquinoline-2,4-dicarboxamide

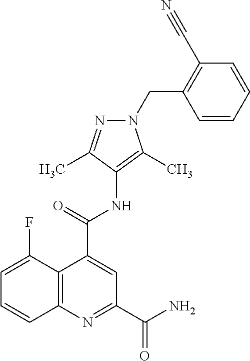 [SMILES: Cc1nn(Cc2ccccc2C#N)c(C)c1NC(=O)c1cc(C(N)=O)nc2cccc(F)c12]

In analogy to example 118), 87 mg (0.38 mmol) 2-[(4-amino-3,5-dimethyl-1H-pyrazol-1-yl)methyl]benzonitrile (intermediate 10C) and 75 mg (0.32 mmol) 2-carbamoyl-5-fluoroquinoline-4-carboxylic acid (intermediate 39A) were reacted to give after purification via preparative HPLC (method 3) 35 mg (23%) of the desired title compound.

¹H-NMR (400 MHz, DMSO d₆) δ (ppm)=2.14 (s, 3H), 2.26 (s, 3H), 5.45 (s, 2H), 7.11 (d, 1H), 7.50-7.58 (m, 1H), 7.69-7.75 (m, 1H), 7.75-7.82 (m, 1H), 7.86-8.01 (m, 3H), 8.30 (s, 1H), 8.33-8.44 (m, 2H), 10.19 (s, 1H).

### Example 268

### 6-bromo-N⁴-[1-(2-cyanobenzyl)-3,5-dimethyl-1H-pyrazol-4-yl]quinoline-2,4-dicarboxamide

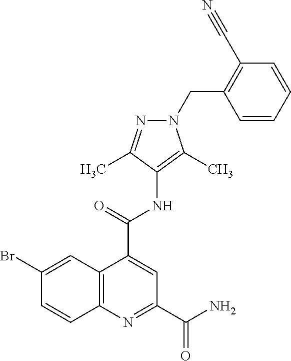 [SMILES: Cc1nn(Cc2ccccc2C#N)c(C)c1NC(=O)c1cc(C(N)=O)nc2ccc(Br)cc12]

In analogy to example 118), 92 mg (0.41 mmol) 2-[(4-amino-3,5-dimethyl-1H-pyrazol-1-yl)methyl]benzonitrile (intermediate 10C) and 100 mg (0.34 mmol) 6-bromo-2-carbamoylquinoline-4-carboxylic acid (intermediate 2A) were reacted to give after purification via preparative HPLC (method 3) and subsequent additional purification via a Biotage chromatography system (10 g snap KP-Sil column, hexane/50-100% ethyl acetate, then ethyl acetate/0-100% methanol) 26 mg (14%) of the desired title compound.

¹H-NMR (400 MHz, DMSO d₆) δ (ppm)=2.14 (s, 3H), 2.26 (s, 3H), 5.45 (s, 2H), 7.12 (d, 1H), 7.49-7.58 (m, 1H), 7.73 (td, 1H), 7.91 (dd, 1H), 7.96 (br. s., 1H), 8.07-8.13 (m, 1H), 8.13-8.19 (m, 1H), 8.39 (s, 1H), 8.43 (br. s., 1H), 8.50 (d, 1H), 10.23 (s, 1H).

### Example 269

### 6-chloro-N⁴-[1-(2-cyanobenzyl)-3,5-dimethyl-1H-pyrazol-4-yl]-8-fluoroquinoline-2,4-dicarboxamide

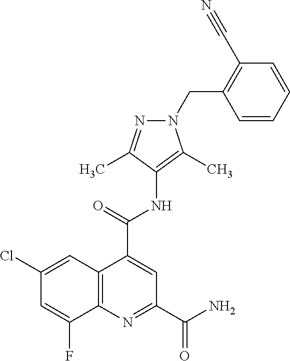 [SMILES: Cc1nn(Cc2ccccc2C#N)c(C)c1NC(=O)c1cc(C(N)=O)nc2c(F)cc(Cl)cc12]

In analogy to example 118), 101 mg (0.45 mmol) 2-[(4-amino-3,5-dimethyl-1H-pyrazol-1-yl)methyl]benzonitrile (intermediate 10C) and 100 mg (0.37 mmol) 2-carbamoyl-6-chloro-8-fluoroquinoline-4-carboxylic acid (intermediate 49A) were reacted to give after purification via preparative HPLC (method 3) 33 mg (17%) of the desired title compound.

¹H-NMR (300 MHz, DMSO d) δ (ppm)=2.11 (s, 3H), 2.24 (s, 3H), 5.43 (s, 2H), 7.10 (d, 1H), 7.52 (t, 1H), 7.71 (t, 1H), 7.90 (d, 1H), 7.99-8.07 (m, 2H), 8.15 (s, 1H), 8.28 (s, 1H), 8.46 (s, 1H), 10.27 (s, 1H).

### Example 270

### 6-chloro-N⁴-[1-(2-cyanobenzyl)-5-methyl-3-(trifluoromethyl)-1H-pyrazol-4-yl]-7-fluoroquinoline-2,4-dicarboxamide

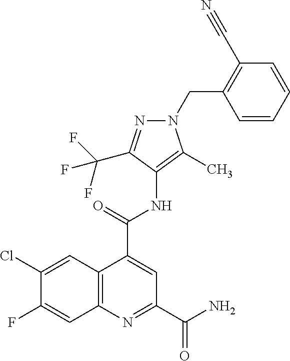 [SMILES: Cc1c(NC(=O)c2cc(C(N)=O)nc3cc(F)c(Cl)cc23)c(C(F)(F)F)nn1Cc1ccccc1C#N]

In analogy to example 118), 94 mg (0.34 mmol) 2-{[4-amino-5-methyl-3-(trifluoromethyl)-1H-pyrazol-1-yl]methyl}benzonitrile (intermediate 48C) and 75 mg (0.28 mmol) 2-carbamoyl-6-chloro-7-fluoroquinoline-4-carboxylic acid (intermediate 32A) were reacted to give after purification via preparative HPLC (method 3) 45 mg (29%) of the desired title compound.

¹H-NMR (400 MHz, DMSO d₆) δ (ppm)=2.36 (s, 3H), 5.67 (s, 2H), 7.24 (d, 1H), 7.59 (td, 1H), 7.78 (td, 1H), 7.95 (dd, 1H), 8.00-8.06 (m, 1H), 8.15 (d, 1H), 8.39 (s, 1H), 8.42 (d, 1H), 8.45 (d, 1H), 10.60 (s, 1H).

### Example 271

### N⁴-[1-(2-cyanobenzyl)-5-methyl-3-(trifluoromethyl)-1H-pyrazol-4-yl]quinoline-2,4-dicarboxamide

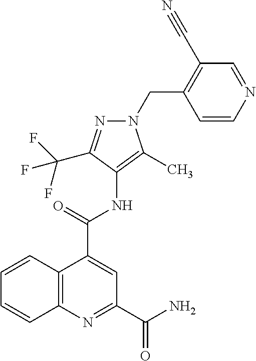 [SMILES: Cc1c(NC(=O)c2cc(C(N)=O)nc3ccccc23)c(C(F)(F)F)nn1Cc1ccncc1C#N]

In analogy to example 118), 117 mg (0.42 mmol) 2-{[4-amino-5-methyl-3-(trifluoromethyl)-1H-pyrazol-1-yl]methyl}benzonitrile (intermediate 48C) and 75 mg (0.28 mmol) 2-carbamoylquinoline-4-carboxylic acid (intermediate 4A) were reacted to give after purification via preparative HPLC (method 3) 53 mg (31%) of the desired title compound.

¹H-NMR (400 MHz, DMSO d) δ (ppm)=2.37 (s, 3H), 5.68 (s, 2H), 7.23 (d, 1H), 7.59 (td, 1H), 7.78 (td, 1H), 7.84 (ddd, 1H), 7.91-7.99 (m, 3H), 8.20-8.25 (m, 2H), 8.29 (s, 1H), 8.42 (d, 1H), 10.47 (s, 1H).

### Example 272

### N⁴-[1-(2-cyanobenzyl)-5-methyl-3-(trifluoromethyl)-1H-pyrazol-4-yl]-7-fluoroquinoline-2,4-dicarboxamide

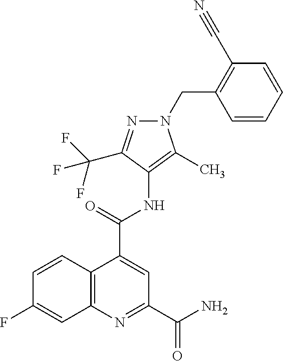 [SMILES: Cc1c(NC(=O)c2cc(C(N)=O)nc3cc(F)ccc23)c(C(F)(F)F)nn1Cc1ccccc1C#N]

In analogy to example 118), 108 mg (0.38 mmol) 2-{[4-amino-5-methyl-3-(trifluoromethyl)-1H-pyrazol-1-yl]methyl}benzonitrile (intermediate 48C) and 75 mg (0.32 mmol) 2-carbamoyl-7-fluoroquinoline-4-carboxylic acid (intermediate 37A) were reacted to give after purification via preparative HPLC (method 3) 65 mg (39%) of the desired title compound.

¹H-NMR (400 MHz, DMSO d) δ (ppm)=2.36 (s, 3H), 5.68 (s, 2H), 7.22 (d, 1H), 7.59 (td, 1H), 7.73-7.86 (m, 2H), 7.91-8.01 (m, 3H), 8.27-8.34 (m, 2H), 8.41 (d, 1H), 10.52 (s, 1H).

### Example 273

### N⁴-[1-(2-cyanobenzyl)-5-methyl-3-(trifluoromethyl)-1H-pyrazol-4-yl]-5-fluoroquinoline-2,4-dicarboxamide

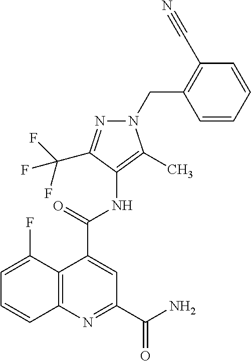 [SMILES: Cc1c(NC(=O)c2cc(C(N)=O)nc3cccc(F)c23)c(C(F)(F)F)nn1Cc1ccccc1C#N]

In analogy to example 118), 108 mg (0.38 mmol) 2-{[4-amino-5-methyl-3-(trifluoromethyl)-1H-pyrazol-1-yl]methyl}benzonitrile (intermediate 48C) and 75 mg (0.32 mmol) 2-carbamoyl-5-fluoroquinoline-4-carboxylic acid (intermediate 39A) were reacted to give after purification via preparative HPLC (method 3) 64 mg (39%) of the desired title compound.

¹H-NMR (400 MHz, DMSO d) δ (ppm)=2.36 (s, 3H), 5.68 (s, 2H), 7.22 (d, 1H), 7.59 (td, 1H), 7.73-7.85 (m, 2H), 7.90-8.02 (m, 3H), 8.27-8.34 (m, 2H), 8.41 (d, 1H), 10.52 (s, 1H).

### Example 274

### 6-bromo-N⁴-[1-(2-cyanobenzyl)-5-methyl-3-(trifluoromethyl)-1H-pyrazol-4-yl]quinoline-2,4-dicarboxamide

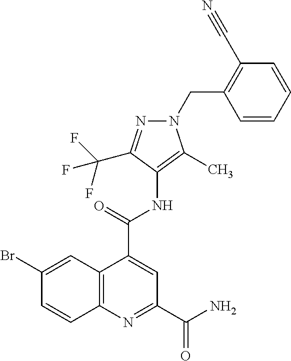 [SMILES: Cc1c(NC(=O)c2cc(C(N)=O)nc3ccc(Br)cc23)c(C(F)(F)F)nn1Cc1ccccc1C#N]

In analogy to example 118), 85 mg (0.31 mmol) 2-{[4-amino-5-methyl-3-(trifluoromethyl)-1H-pyrazol-1-yl]methyl}benzonitrile (intermediate 48C) and 75 mg (0.25 mmol) 6-bromo-2-carbamoylquinoline-4-carboxylic acid (intermediate 2A) were reacted to give after purification via preparative HPLC (method 3) 39 mg (26%) of the desired title compound.

¹H-NMR (400 MHz, DMSO d₆) δ (ppm)=2.36 (s, 3H), 5.67 (s, 2H), 7.24 (d, 1H), 7.56-7.61 (m, 1H), 7.75-7.81 (m, 1H), 7.93-8.00 (m, 2H), 8.10 (dd, 1H), 8.16 (d, 1H), 8.37 (s, 1H), 8.41-8.48 (m, 2H), 10.55 (s, 1H).

### Example 275

### 7-fluoro-N⁴-[1-(4-methoxybenzyl)-3,5-dimethyl-1H-pyrazol-4-yl]quinoline-2,4-dicarboxamide

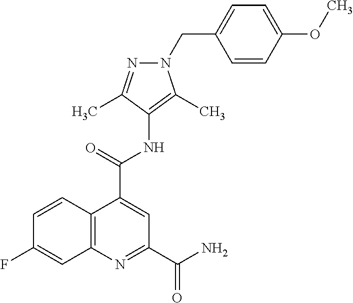 [SMILES: COc1ccc(Cn2nc(C)c(NC(=O)c3cc(C(N)=O)nc4cc(F)ccc34)c2C)cc1]

In analogy to example 118), 119 mg (0.51 mmol) 1-(4-methoxybenzyl)-3,5-dimethyl-1H-pyrazol-4-amine (intermediate 11C) and 100 mg (0.43 mmol) 2-carbamoyl-7-fluoroquinoline-4-carboxylic acid (intermediate 37A) were reacted to give after purification via preparative HPLC (method 3) 100 mg (48%) of the desired title compound.

¹H-NMR (300 MHz, DMSO d₆) δ (ppm)=2.12 (s, 3H), 2.16 (s, 3H), 3.72 (s, 3H), 5.16 (s, 2H), 6.90 (d, 2H), 7.15 (d, 2H), 7.76 (td, 1H), 7.90 (dd, 1H), 7.95 (br. s., 1H), 8.26 (s, 1H), 8.33 (dd, 1H), 8.38 (br. s., 1H), 10.09 (s, 1H).

### Example 276

### 6-chloro-8-fluoro-N⁴-[1-(4-methoxybenzyl)-3,5-dimethyl-1H-pyrazol-4-yl]quinoline-2,4-dicarboxamide

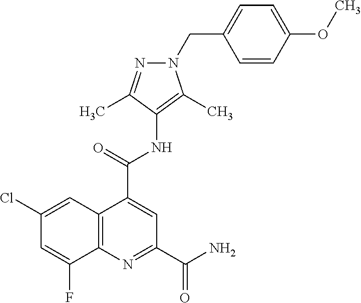 [SMILES: COc1ccc(Cn2nc(C)c(NC(=O)c3cc(C(N)=O)nc4c(F)cc(Cl)cc34)c2C)cc1]

In analogy to example 118), 103 mg (0.48 mmol) 1-(4-methoxybenzyl)-3,5-dimethyl-1H-pyrazol-4-amine (intermediate 11C) and 100 mg (0.37 mmol) 2-carbamoyl-6-chloro-8-fluoroquinoline-4-carboxylic acid (intermediate 49A) were reacted to give after purification via preparative HPLC (method 3) 28 mg (15%) of the desired title compound.

¹H-NMR (300 MHz, DMSO d₆) δ (ppm)=2.11 (s, 3H), 2.16 (s, 3H), 3.72 (s, 3H), 5.16 (s, 2H), 6.91 (d, 2H), 7.15 (d, 2H), 7.97-8.07 (m, 2H), 8.13 (s, 1H), 8.27 (br. s., 1H), 8.43 (s, 1H), 10.19 (s, 1H).

### Example 277

### N⁴-[1-(4-methoxybenzyl)-5-methyl-3-(trifluoromethyl)-1H-pyrazol-4-yl]quinoline-2,4-dicarboxamide

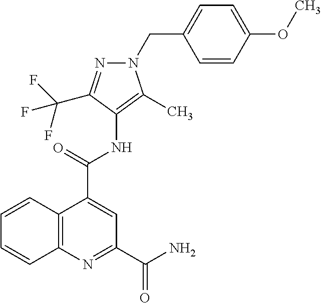 [SMILES: COc1ccc(Cn2nc(C(F)(F)F)c(NC(=O)c3cc(C(N)=O)nc4ccccc34)c2C)cc1]

In analogy to example 118), 198 mg (0.69 mmol) 1-(4-methoxybenzyl)-5-methyl-3-(trifluoromethyl)-1H-pyrazol-4-amine (intermediate 49C) and 125 mg (0.58 mmol) 2-carbamoylquinoline-4-carboxylic acid (intermediate 4A) were reacted to give after purification via preparative HPLC (method 3) 126 mg (44%) of the desired title compound.

¹H-NMR (300 MHz, DMSO d₆) δ (ppm)=2.27 (s, 3H), 3.74 (s, 3H), 5.38 (s, 2H), 6.95 (d, 2H), 7.22 (d, 2H), 7.77-7.86 (m, 1H), 7.88-7.98 (m, 2H), 8.16-8.29 (m, 3H), 8.40 (br. s., 1H), 10.37 (s, 1H).

### Example 278

### 6-chloro-7-fluoro-N⁴-[1-(4-methoxybenzyl)-5-methyl-3-(trifluoromethyl)-1H-pyrazol-4-yl]quinoline-2,4-dicarboxamide

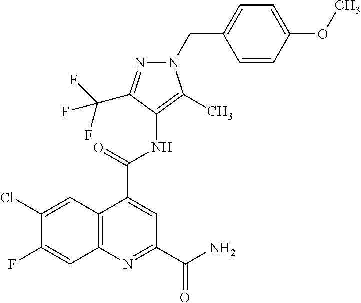 [SMILES: COc1ccc(Cn2nc(C(F)(F)F)c(NC(=O)c3cc(C(N)=O)nc4cc(F)c(Cl)cc34)c2C)cc1]

In analogy to example 118), 159 mg (0.56 mmol) 1-(4-methoxybenzyl)-5-methyl-3-(trifluoromethyl)-1H-pyrazol-4-amine (intermediate 49C) and 125 mg (0.47 mmol) 2-carbamoyl-6-chloro-7-fluoroquinoline-4-carboxylic acid (intermediate 32A) were reacted to give after purification via preparative HPLC (method 3) 93 mg (35%) of the desired title compound.

¹H-NMR (300 MHz, DMSO d₆) δ (ppm)=2.26 (s, 3H), 3.74 (s, 3H), 5.38 (s, 2H), 6.95 (d, 2H), 7.22 (d, 2H), 8.01 (s, 1H), 8.13 (d, 1H), 8.34 (s, 1H), 8.38-8.45 (m, 2H), 10.50 (s, 1H).

### Example 279

### 6,7-difluoro-N⁴-[1-(4-methoxybenzyl)-5-methyl-3-(trifluoromethyl)-1H-pyrazol-4-yl]quinoline-2,4-dicarboxamide

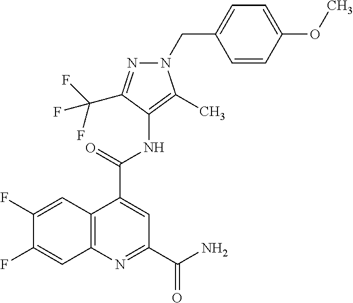 [SMILES: COc1ccc(Cn2nc(C(F)(F)F)c(NC(=O)c3cc(C(N)=O)nc4cc(F)c(F)cc34)c2C)cc1]

In analogy to example 118), 170 mg (0.60 mmol) 1-(4-methoxybenzyl)-5-methyl-3-(trifluoromethyl)-1H-pyrazol-4-amine (intermediate 49C) and 125 mg (0.50 mmol) 2-carbamoyl-6,7-difluoroquinoline-4-carboxylic acid (intermediate 3A) were reacted to give after purification via preparative HPLC (method 3) 29 mg (11%) of the desired title compound.

¹H-NMR (300 MHz, DMSO d₆) δ (ppm)=2.26 (s, 3H), 3.74 (s, 3H), 5.38 (s, 2H), 6.95 (d, 2H), 7.22 (d, 2H), 7.98 (s, 1H), 8.09-8.21 (m, 2H), 8.35 (s, 1H), 8.38 (br. s., 1H), 10.48 (s, 1H).

### Example 280

### N⁴-{1-[(6-methoxypyridin-3-yl)methyl]-5-methyl-3-(trifluoromethyl)-1H-pyrazol-4-yl}quinoline-2,4-dicarboxamide

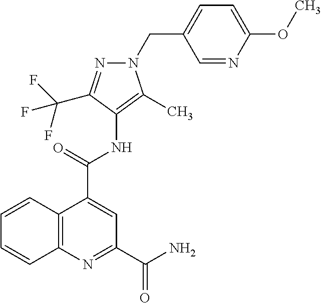 [SMILES: COc1ccc(Cn2nc(C(F)(F)F)c(NC(=O)c3cc(C(N)=O)nc4ccccc34)c2C)cn1]

In analogy to example 118), 156 mg (0.56 mmol) 1-[(6-methoxypyridin-3-yl)methyl]-5-methyl-3-(trifluoromethyl)-1H-pyrazol-4-amine (intermediate 43C) and 100 mg (0.46 mmol) 2-carbamoylquinoline-4-carboxylic acid (intermediate 4A) were reacted to give after purification via preparative HPLC (method 3) 96 mg (42%) of the desired title compound.

¹H-NMR (400 MHz, DMSO d₆) δ (ppm)=2.32 (s, 3H), 3.84 (s, 3H), 5.42 (s, 2H), 6.86 (d, 1H), 7.63 (dd, 1H), 7.78-7.85 (m, 1H), 7.90 (d, 1H), 7.94 (ddd, 1H), 8.15-8.23 (m, 3H), 8.25 (s, 1H), 8.35-8.42 (m, 1H), 10.37 (s, 1H).

### Example 281

### 6-chloro-7-fluoro-N⁴-{1-[(6-methoxypyridin-3-yl)methyl]-5-methyl-3-(trifluoromethyl)-1H-pyrazol-4-yl}quinoline-2,4-dicarboxamide

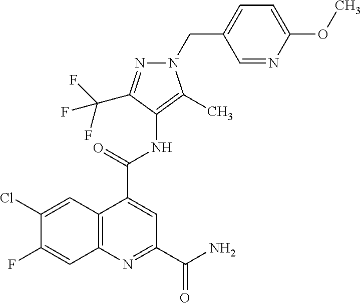 [SMILES: COc1ccc(Cn2nc(C(F)(F)F)c(NC(=O)c3cc(C(N)=O)nc4cc(F)c(Cl)cc34)c2C)cn1]

In analogy to example 118), 128 mg (0.45 mmol) 1-[(6-methoxypyridin-3-yl)methyl]-5-methyl-3-(trifluoromethyl)-1H-pyrazol-4-amine (intermediate 43C) and 100 mg (0.37 mmol) 2-carbamoyl-6-chloro-7-fluoroquinoline-4-carboxylic acid (intermediate 32A) were reacted to give after purification via preparative HPLC (method 3) 72 mg (35%) of the desired title compound.

¹H-NMR (300 MHz, DMSO d₆) δ (ppm)=2.31 (s, 3H), 3.84 (s, 3H), 5.42 (s, 2H), 6.86 (d, 1H), 7.64 (dd, 1H), 8.01 (br. s., 1H), 8.07-8.21 (m, 2H), 8.32-8.46 (m, 3H), 10.52 (s, 1H).

### Example 282

### N⁴-{1-[(6-methoxypyridin-3-yl)methyl]-3,5-dimethyl-1H-pyrazol-4-yl}quinoline-2,4-dicarboxamide

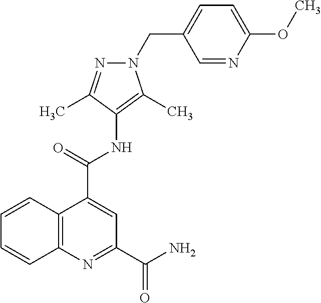 [SMILES: COc1ccc(Cn2nc(C)c(NC(=O)c3cc(C(N)=O)nc4ccccc34)c2C)cn1]

In analogy to example 118), 129 mg (0.56 mmol) 1-[(6-methoxypyridin-3-yl)methyl]-3,5-dimethyl-1H-pyrazol-4-amine (intermediate 50C) and 100 mg (0.46 mmol) 2-carbamoylquinoline-4-carboxylic acid (intermediate 4A) were reacted to give after purification via preparative HPLC (method 3) 92 mg (44%) of the desired title compound.

¹H-NMR (400 MHz, DMSO d₆) δ (ppm)=2.15 (s, 3H), 2.24 (s, 3H), 3.85 (s, 3H), 5.22 (s, 2H), 6.83 (d, 1H), 7.59 (dd, 1H), 7.83 (ddd, 1H), 7.88-7.98 (m, 2H), 8.10 (d, 1H), 8.22 (d, 1H), 8.25-8.28 (m, 1H), 8.29 (s, 1H), 8.37-8.43 (m, 1H), 10.06 (s, 1H).

### Example 283

### 6-chloro-7-fluoro-N⁴-{1-[(6-methoxypyridin-3-yl)methyl]-3,5-dimethyl-1H-pyrazol-4-yl}quinoline-2,4-dicarboxamide

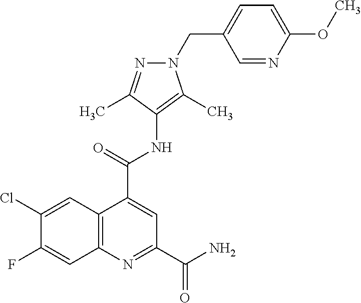 [SMILES: COc1ccc(Cn2nc(C)c(NC(=O)c3cc(C(N)=O)nc4cc(F)c(Cl)cc34)c2C)cn1]

In analogy to example 118), 104 mg (0.45 mmol) 1-[(6-methoxypyridin-3-yl)methyl]-3,5-dimethyl-1H-pyrazol-4-amine (intermediate 50C) and 100 mg (0.37 mmol) 2-carbamoyl-6-chloro-7-fluoroquinoline-4-carboxylic acid (intermediate 32A) were reacted to give after purification via preparative HPLC (method 3) 32 mg (17%) of the desired title compound.

¹H-NMR (300 MHz, DMSO d₆) δ (ppm)=2.11 (s, 3H), 2.20 (s, 3H), 3.83 (s, 3H), 5.19 (s, 2H), 6.81 (d, 1H), 7.57 (dd, 1H), 7.99 (br. s., 1H), 8.05-8.18 (m, 2H), 8.35-8.43 (m, 2H), 8.50 (d, 1H), 10.19 (s, 1H).

### Example 284

### N⁴-{1-[3-(4-methoxyphenyl)propyl]-3,5-dimethyl-1H-pyrazol-4-yl}quinoline-2,4-dicarboxamide

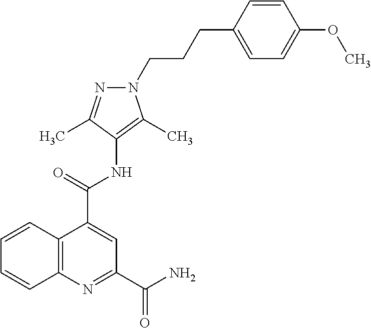 [SMILES: COc1ccc(CCCn2nc(C)c(NC(=O)c3cc(C(N)=O)nc4ccccc34)c2C)cc1]

In analogy to example 118), 144 mg (0.56 mmol) 1-[3-(4-methoxyphenyl)propyl]-3,5-dimethyl-1H-pyrazol-4-amine (intermediate 44C) and 100 mg (0.46 mmol) 2-carbamoylquinoline-4-carboxylic acid (intermediate 4A) were reacted to give after purification via preparative HPLC (method 4) 114 mg (48%) of the desired title compound.

¹H-NMR (400 MHz, DMSO d₆) δ (ppm)=2.01 (tt, 2H), 2.14 (s, 3H), 2.19 (s, 3H), 2.56 (t, 2H), 3.73 (s, 3H), 3.97 (t, 2H), 6.84-6.90 (m, 2H), 7.16 (dd, 2H), 7.83 (ddd, 1H), 7.89-7.98 (m, 2H), 8.20-8.30 (m, 3H), 8.38-8.44 (m, 1H), 10.03 (s, 1H).

### Example 285

### 6-chloro-N⁴-{1-[(3-cyanopyridin-4-yl)methyl]-3,5-dimethyl-1H-pyrazol-4-yl}-7-fluoroquinoline-2,4-dicarboxamide

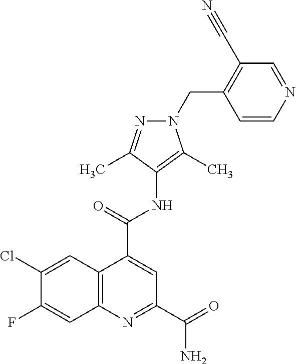 [SMILES: Cc1nn(Cc2ccncc2C#N)c(C)c1NC(=O)c1cc(C(N)=O)nc2cc(F)c(Cl)cc12]

In analogy to example 118), 102 mg (0.45 mmol) 4-[(4-amino-3,5-dimethyl-1H-pyrazol-1-yl)methyl]nicotinonitrile (intermediate 45C) and 100 mg (0.40 mmol) 2-carbamoyl-6-chloro-7-fluoroquinoline-4-carboxylic acid (intermediate 32A) were reacted to give after purification via preparative HPLC (method 3) 31 mg (18%) of the desired title compound.

¹H-NMR (400 MHz, DMSO d₆) δ (ppm)=2.14 (s, 3H), 2.25 (s, 3H), 5.52 (s, 2H), 6.98 (d, 1H), 8.01 (d, 1H), 8.15 (d, 1H), 8.39-8.43 (m, 2H), 8.54 (d, 1H), 8.83 (d, 1H), 9.07 (d, 1H), 10.32 (s, 1H).

### Example 286

### N⁴-{1-[(3-cyanopyridin-4-yl)methyl]-3,5-dimethyl-1H-pyrazol-4-yl}quinoline-2,4-dicarboxamide

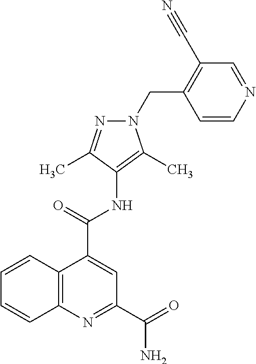 [SMILES: Cc1nn(Cc2ccncc2C#N)c(C)c1NC(=O)c1cc(C(N)=O)nc2ccccc12]

In analogy to example 118), 126 mg (0.56 mmol) 4-[(4-amino-3,5-dimethyl-1H-pyrazol-1-yl)methyl]nicotinonitrile (intermediate 45C) and 100 mg (0.46 mmol) 2-carbamoylquinoline-4-carboxylic acid (intermediate 4A) were reacted to give after purification via preparative HPLC (method 3) 94 mg (44%) of the desired title compound.

¹H-NMR (400 MHz, DMSO d₆) δ (ppm)=2.16 (s, 3H), 2.26 (s, 3H), 5.52 (s, 2H), 6.98 (d, 1H), 7.83 (ddd, 1H), 7.88-7.99 (m, 2H), 8.23 (d, 1H), 8.26-8.30 (m, 1H), 8.31 (s, 1H), 8.41 (d, 1H), 8.83 (d, 1H), 9.07 (s, 1H), 10.17 (s, 1H).

### Example 287

### 6-chloro-N⁴-{1-[(3-cyanopyridin-4-yl)methyl]-5-methyl-3-(trifluoromethyl)-1H-pyrazol-4-yl}-7-fluoroquinoline-2,4-dicarboxamide

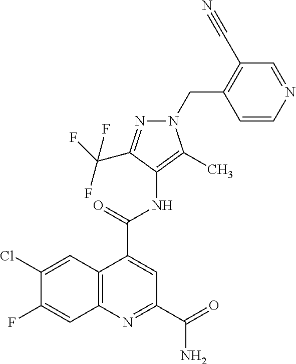 [SMILES: Cc1c(NC(=O)c2cc(C(N)=O)nc3cc(F)c(Cl)cc23)c(C(F)(F)F)nn1Cc1ccncc1C#N]

In analogy to example 118), 126 mg (0.45 mmol) 4-{[4-amino-5-methyl-3-(trifluoromethyl)-1H-pyrazol-1-yl]methyl}nicotinonitrile (intermediate 51C) and 100 mg (0.37 mmol) 2-carbamoyl-6-chloro-7-fluoroquinoline-4-carboxylic acid (intermediate 32A) were reacted to give after purification via preparative HPLC (method 3) 23 mg (11%) of the desired title compound.

¹H-NMR (400 MHz, DMSO d₆) δ (ppm)=2.34 (s, 3H), 5.76 (s, 2H), 7.08 (d, 1H), 8.03 (s, 1H), 8.16 (d, 1H), 8.40 (s, 1H), 8.42 (d, 1H), 8.45 (d, 1H), 8.89 (d, 1H), 9.11 (s, 1H), 10.64 (s, 1H).

### Example 288

### N⁴-{1-[(3-cyanopyridin-4-yl)methyl]-5-methyl-3-(trifluoromethyl)-1H-pyrazol-4-yl}quinoline-2,4-dicarboxamide

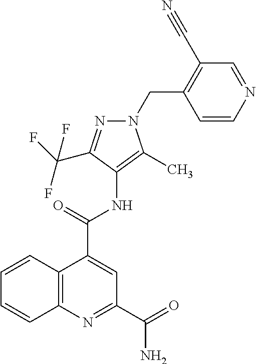 [SMILES: Cc1c(NC(=O)c2cc(C(N)=O)nc3ccccc23)c(C(F)(F)F)nn1Cc1ccncc1C#N]

In analogy to example 118), 156 mg (0.56 mmol) 4-{[4-amino-5-methyl-3-(trifluoromethyl)-1H-pyrazol-1-yl]methyl}nicotinonitrile (intermediate 51C) and 100 mg (0.46 mmol) 2-carbamoylquinoline-4-carboxylic acid (intermediate 4A) were reacted to give after purification via preparative HPLC (method 3) 75 mg (33%) of the desired title compound.

¹H-NMR (400 MHz, DMSO d₆) δ (ppm)=2.35 (s, 3H), 5.77 (s, 2H), 7.07 (d, 1H), 7.80-7.88 (m, 1H), 7.90-8.01 (m, 2H), 8.23 (dd, 2H), 8.30 (s, 1H), 8.42 (s, 1H), 8.88 (d, 1H), 9.11 (s, 1H), 10.51 (s, 1H).

### Example 289

### N⁴-{1-[(6-cyanopyridin-3-yl)methyl]-5-methyl-3-(trifluoromethyl)-1H-pyrazol-4-yl}quinoline-2,4-dicarboxamide

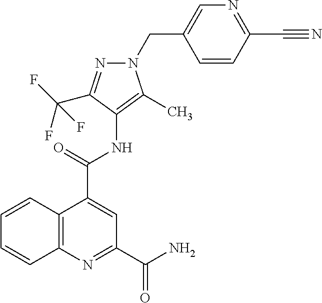 [SMILES: Cc1c(NC(=O)c2cc(C(N)=O)nc3ccccc23)c(C(F)(F)F)nn1Cc1ccc(C#N)nc1]

In analogy to example 118), 115 mg (0.41 mmol) 5-{[4-amino-5-methyl-3-(trifluoromethyl)-1H-pyrazol-1-yl]methyl}pyridine-2-carbonitrile (intermediate 52C) and 74 mg (0.34 mmol) 2-carbamoylquinoline-4-carboxylic acid (intermediate 4A) were reacted to give after purification via preparative HPLC (method 3) 38 mg (23%) of the desired title compound.

¹H-NMR (400 MHz, DMSO d₆) δ (ppm)=2.33 (s, 3H), 5.66 (s, 2H), 7.79-7.86 (m, 2H), 7.90 (d, 1H), 7.94 (ddd, 1H), 8.09 (dd, 1H), 8.18-8.23 (m, 2H), 8.26 (s, 1H), 8.39 (d, 1H), 8.69 (d, 1H), 10.42 (s, 1H).

### Example 290

### N⁴-{1-[(6-cyanopyridin-3-yl)methyl]-5-methyl-3-(trifluoromethyl)-1H-pyrazol-4-yl}-7-fluoroquinoline-2,4-dicarboxamide

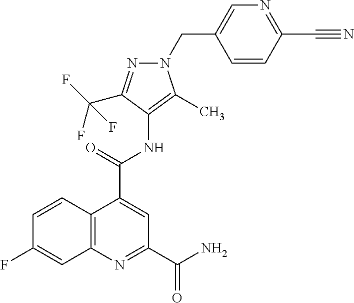 [SMILES: Cc1c(NC(=O)c2cc(C(N)=O)nc3cc(F)ccc23)c(C(F)(F)F)nn1Cc1ccc(C#N)nc1]

In analogy to example 118), 108 mg (0.38 mmol) 5-{[4-amino-5-methyl-3-(trifluoromethyl)-1H-pyrazol-1-yl]methyl}pyridine-2-carbonitrile (intermediate 52C) and 75 mg (0.32 mmol) 2-carbamoyl-7-fluoroquinoline-4-carboxylic acid (intermediate 37A) were reacted to give after purification via preparative HPLC (method 4) 55 mg (23%) of the desired title compound.

¹H-NMR (400 MHz, DMSO d₆) δ (ppm)=2.34 (s, 3H), 5.68 (s, 2H), 7.78-7.88 (m, 2H), 7.94 (dd, 1H), 7.98 (d, 1H), 8.11 (dd, 1H), 8.27-8.33 (m, 2H), 8.41 (d, 1H), 8.70 (d, 1H), 10.50 (s, 1H).

### Example 291

### N⁴-{1-[(6-cyanopyridin-3-yl)methyl]-5-methyl-3-(trifluoromethyl)-1H-pyrazol-4-yl}-5-fluoroquinoline-2,4-dicarboxamide

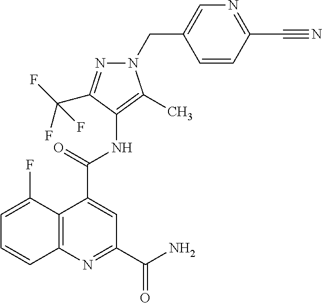 [SMILES: Cc1c(NC(=O)c2cc(C(N)=O)nc3cccc(F)c23)c(C(F)(F)F)nn1Cc1ccc(C#N)nc1]

In analogy to example 118), 108 mg (0.38 mmol) 5-{[4-amino-5-methyl-3-(trifluoromethyl)-1H-pyrazol-1-yl]methyl}pyridine-2-carbonitrile (intermediate 52C) and 75 mg (0.32 mmol) 2-carbamoyl-5-fluoroquinoline-4-carboxylic acid (intermediate 39A) were reacted to give after purification via preparative HPLC (method 4) 67 mg (23%) of the desired title compound.

¹H-NMR (400 MHz, DMSO d₆) δ (ppm)=2.34 (s, 3H), 5.68 (s, 2H), 7.78-7.87 (m, 2H), 7.94 (dd, 1H), 7.98 (d, 1H), 8.05-8.17 (m, 1H), 8.26-8.33 (m, 2H), 8.41 (d, 1H), 8.70 (d, 1H), 10.50 (s, 1H).

### Example 292

### N⁴-{1-[(6-cyanopyridin-3-yl)methyl]-3,5-dimethyl-1H-pyrazol-4-yl}-7-fluoroquinoline-2,4-dicarboxamide

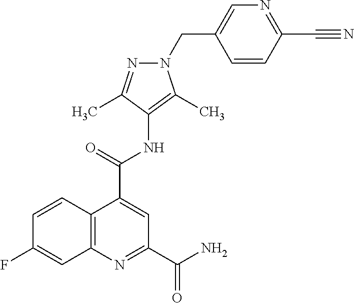 [SMILES: Cc1nn(Cc2ccc(C#N)nc2)c(C)c1NC(=O)c1cc(C(N)=O)nc2cc(F)ccc12]

In analogy to example 118), 116 mg (0.51 mmol) 5-[(4-amino-3,5-dimethyl-1H-pyrazol-1-yl)methyl]pyridine-2-carbonitrile (intermediate 29C) and 100 mg (0.43 mmol) 2-carbamoyl-7-fluoroquinoline-4-carboxylic acid (intermediate 37A) were reacted to give after purification via preparative HPLC (method 3) 37 mg (17%) of the desired title compound.

¹H-NMR (400 MHz, DMSO d₆) δ (ppm)=2.15 (s, 3H), 2.23 (s, 3H), 5.45 (s, 2H), 7.74-7.84 (m, 2H), 7.87-7.95 (m, 1H), 7.97 (s, 1H), 8.06 (d, 1H), 8.29 (s, 1H), 8.34-8.42 (m, 2H), 8.62 (d, 1H), 10.17 (s, 1H).

### Example 293

### 6-bromo-N⁴-{1-[(6-cyanopyridin-3-yl)methyl]-3,5-dimethyl-1H-pyrazol-4-yl}quinoline-2,4-dicarboxamide

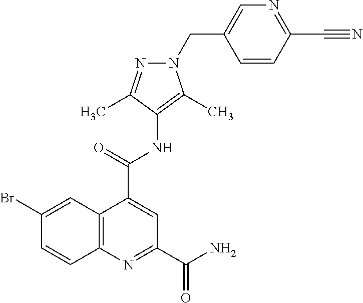 [SMILES: Cc1nn(Cc2ccc(C#N)nc2)c(C)c1NC(=O)c1cc(C(N)=O)nc2ccc(Br)cc12]

In analogy to example 118), 92 mg (0.41 mmol) 5-[(4-amino-3,5-dimethyl-1H-pyrazol-1-yl)methyl]pyridine-2-carbonitrile (intermediate 29C) and 100 mg (0.34 mmol) 6-bromo-2-carbamoylquinoline-4-carboxylic acid (intermediate 2A) were reacted to give after purification via preparative HPLC (method 3) 27 mg (14%) of the desired title compound.

¹H-NMR (400 MHz, DMSO d₆) δ (ppm)=2.15 (s, 3H), 2.23 (s, 3H), 5.45 (s, 2H), 7.78 (dd, 1H), 7.96 (br. s., 1H), 8.01-8.19 (m, 3H), 8.38 (s, 1H), 8.43 (s, 1H), 8.49 (d, 1H), 8.63 (d, 1H), 10.22 (s, 1H).

### Example 294

### 6-chloro-N⁴-{1-[2-(4-cyanophenoxy)ethyl]-3,5-dimethyl-1H-pyrazol-4-yl}-7-fluoroquinoline-2,4-dicarboxamide

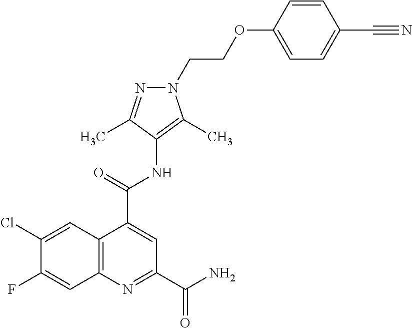 [SMILES: Cc1nn(CCOc2ccc(C#N)cc2)c(C)c1NC(=O)c1cc(C(N)=O)nc2cc(F)c(Cl)cc12]

In analogy to example 118), 114 mg (0.45 mmol) 3,5-dimethyl-1-(2-phenoxyethyl)-1H-pyrazol-4-amine (intermediate 46C) and 100 mg (0.37 mmol) 2-carbamoyl-6-chloro-7-fluoroquinoline-4-carboxylic acid (intermediate 32A) were reacted to give after filtration a solid raw material which further purified by washing with ethyl acetate to give 107 mg (51%) of the desired title compound.

¹H-NMR (400 MHz, DMSO d₆) δ (ppm)=2.10 (s, 3H), 2.25 (s, 3H), 4.36-4.46 (m, 4H), 7.07-7.13 (m, 2H), 7.72-7.77 (m, 2H), 7.98 (s, 1H), 8.12 (d, 1H), 8.35-8.40 (m, 2H), 8.49 (d, 1H), 10.17 (s, 1H).

### Example 295

### N⁴-{1-[2-(4-cyanophenoxy)ethyl]-3,5-dimethyl-1H-pyrazol-4-yl}quinoline-2,4-dicarboxamide

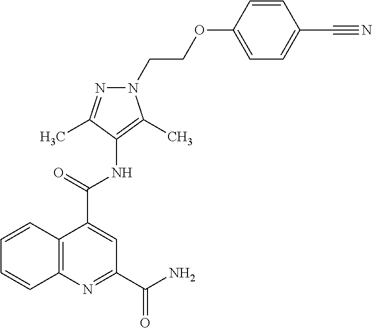 [SMILES: Cc1nn(CCOc2ccc(C#N)cc2)c(C)c1NC(=O)c1cc(C(N)=O)nc2ccccc12]

In analogy to example 118), 142 mg (0.56 mmol) 3,5-dimethyl-1-(2-phenoxyethyl)-1H-pyrazol-4-amine (intermediate 46C) and 100 mg (0.46 mmol) 2-carbamoylquinoline-4-carboxylic acid (intermediate 4A) were reacted to give after purification via preparative HPLC (method 4) 47 mg (21%) of the desired title compound.

¹H-NMR (400 MHz, DMSO d₆) δ (ppm)=2.12 (s, 3H), 2.26 (s, 3H), 4.36-4.46 (m, 4H), 7.08-7.13 (m, 2H), 7.72-7.77 (m, 2H), 7.78-7.84 (m, 1H), 7.88 (s, 1H), 7.93 (ddd, 1H), 8.20 (d, 1H), 8.24 (d, 1H), 8.27 (s, 1H), 8.37 (d, 1H), 10.02 (s, 1H).

### Example 296

### 6-chloro-N⁴-[1-(4-cyano-2-fluorobenzyl)-3,5-dimethyl-1H-pyrazol-4-yl]-7-fluoroquinoline-2,4-dicarboxamide

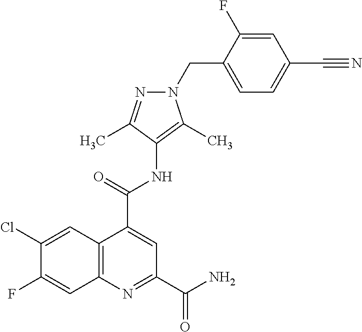 [SMILES: Cc1nn(Cc2ccc(C#N)cc2F)c(C)c1NC(=O)c1cc(C(N)=O)nc2cc(F)c(Cl)cc12]

In analogy to example 118), 142 mg (0.55 mmol) 4-[(4-amino-3,5-dimethyl-1H-pyrazol-1-yl)methyl]-3-fluorobenzonitrile (intermediate 47C) and 124 mg (0.46 mmol) 2-carbamoyl-6-chloro-7-fluoroquinoline-4-carboxylic acid (intermediate 32A) were reacted to give after filtration 105 mg (44%) of the desired title compound.

¹H-NMR (400 MHz, DMSO d) δ (ppm)=2.12 (s, 3H), 2.22 (s, 3H), 5.40 (s, 2H), 7.17 (t, 1H), 7.73 (dd, 1H), 7.92 (dd, 1H), 8.01 (br. s., 1H), 8.14 (d, 1H), 8.38-8.45 (m, 2H), 8.53 (d, 1H), 10.26 (s, 1H).

### Example 297

### N⁴-[1-(4-cyano-2-fluorobenzyl)-3,5-dimethyl-1H-pyrazol-4-yl]quinoline-2,4-dicarboxamide

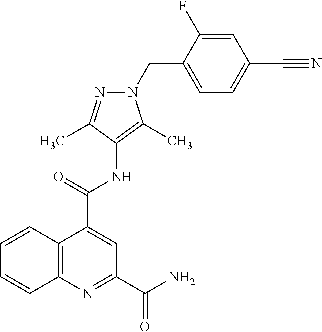 [SMILES: Cc1nn(Cc2ccc(C#N)cc2F)c(C)c1NC(=O)c1cc(C(N)=O)nc2ccccc12]

In analogy to example 118), 166 mg (0.65 mmol) 4-[(4-amino-3,5-dimethyl-1H-pyrazol-1-yl)methyl]-3-fluorobenzonitrile (intermediate 47C) and 116 mg (0.54 mmol) 2-carbamoylquinoline-4-carboxylic acid (intermediate 4A) were reacted to give after purification via preparative HPLC (method 3) 113 mg (45%) of the desired title compound.

¹H-NMR (400 MHz, DMSO d₆) δ (ppm)=2.14 (s, 3H), 2.24 (s, 3H), 5.40 (s, 2H), 7.16 (t, 1H), 7.73 (dd, 1H), 7.80-7.86 (m, 1H), 7.88-7.98 (m, 3H), 8.22 (d, 1H), 8.27 (d, 1H), 8.30 (s, 1H), 8.39-8.43 (m, 1H), 10.12 (s, 1H).

### Example 298

### N⁴-[1-(4-cyano-2-fluorobenzyl)-3,5-dimethyl-1H-pyrazol-4-yl]-6,7-difluoroquinoline-2,4-dicarboxamide

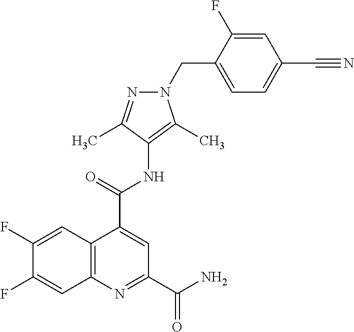 [SMILES: Cc1nn(Cc2ccc(C#N)cc2F)c(C)c1NC(=O)c1cc(C(N)=O)nc2cc(F)c(F)cc12]

In analogy to example 118), 152 mg (0.59 mmol) 4-[(4-amino-3,5-dimethyl-1H-pyrazol-1-yl)methyl]-3-fluorobenzonitrile (intermediate 47C) and 124 mg (0.49 mmol) 2-carbamoyl-6,7-difluoroquinoline-4-carboxylic acid (intermediate 3A) were reacted to give after purification via preparative HPLC (method 3) 35 mg (14%) of the desired title compound.

¹H-NMR (400 MHz, DMSO d₆) δ (ppm)=2.12 (s, 3H), 2.22 (s, 3H), 5.40 (s, 2H), 7.16 (t, 1H), 7.73 (dd, 1H), 7.92 (dd, 1H), 7.99 (s, 1H), 8.18 (dd, 1H), 8.26 (dd, 1H), 8.39 (s, 1H), 8.41 (s, 1H), 10.24 (s, 1H).

### Example 299

### 6-bromo-N⁴-[1-(4-cyano-2-fluorobenzyl)-3,5-dimethyl-1H-pyrazol-4-yl]quinoline-2,4-dicarboxamide

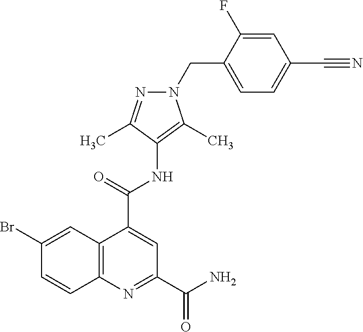 [SMILES: Cc1nn(Cc2ccc(C#N)cc2F)c(C)c1NC(=O)c1cc(C(N)=O)nc2ccc(Br)cc12]

In analogy to example 118), 99 mg (0.41 mmol) 4-[(4-amino-3,5-dimethyl-1H-pyrazol-1-yl)methyl]-3-fluorobenzonitrile (intermediate 47C) and 100 mg (0.34 mmol) 6-bromo-2-carbamoylquinoline-4-carboxylic acid (intermediate 2A) were reacted to give after purification via preparative HPLC (method 3) 47 mg (25%) of the desired title compound.

¹H-NMR (400 MHz, DMSO d₆) δ (ppm)=2.12 (s, 3H), 2.21 (s, 3H), 5.38 (s, 2H), 7.16 (t, 1H), 7.71 (dd, 1H), 7.90 (dd, 1H), 7.93 (d, 1H), 8.07 (dd, 1H), 8.14 (d, 1H), 8.37 (s, 1H), 8.40 (d, 1H), 8.48 (d, 1H), 10.19 (s, 1H).

### Example 300

### N⁴-[1-(4-cyano-2-fluorobenzyl)-3,5-dimethyl-1H-pyrazol-4-yl]-7-fluoroquinoline-2,4-dicarboxamide

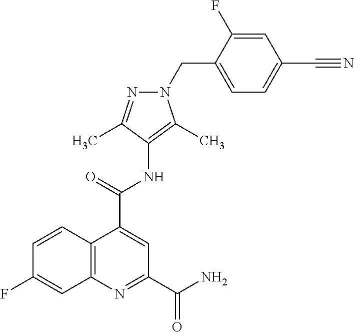 [SMILES: Cc1nn(Cc2ccc(C#N)cc2F)c(C)c1NC(=O)c1cc(C(N)=O)nc2cc(F)ccc12]

In analogy to example 118), 94 mg (0.38 mmol) 4-[(4-amino-3,5-dimethyl-1H-pyrazol-1-yl)methyl]-3-fluorobenzonitrile (intermediate 47C) and 75 mg (0.32 mmol) 2-carbamoyl-7-fluoroquinoline-4-carboxylic acid (intermediate 37A) were reacted to give after purification via preparative HPLC (method 3) 40 mg (40%) of the desired title compound.

¹H-NMR (400 MHz, DMSO d₆) δ (ppm)=2.12 (s, 3H), 2.22 (s, 3H), 5.38 (s, 2H), 7.15 (t, 1H), 7.71 (dd, 1H), 7.77 (ddd, 1H), 7.88-7.95 (m, 3H), 8.28 (s, 1H), 8.32-8.39 (m, 2H), 10.14 (s, 1H).

### Example 301

### N⁴-[1-(4-cyano-2-fluorobenzyl)-3,5-dimethyl-1H-pyrazol-4-yl]-5-fluoroquinoline-2,4-dicarboxamide

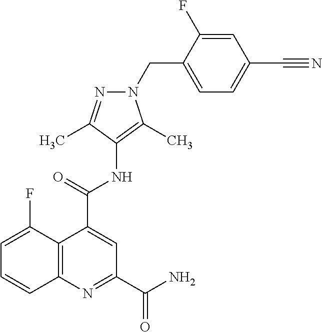 [SMILES: Cc1nn(Cc2ccc(C#N)cc2F)c(C)c1NC(=O)c1cc(C(N)=O)nc2cccc(F)c12]

In analogy to example 118), 94 mg (0.38 mmol) 4-[(4-amino-3,5-dimethyl-1H-pyrazol-1-yl)methyl]-3-fluorobenzonitrile (intermediate 47C) and 75 mg (0.32 mmol) 2-carbamoyl-5-fluoroquinoline-4-carboxylic acid (intermediate 39A) were reacted to give after purification via preparative HPLC (method 3) 63 mg (39%) of the desired title compound.

¹H-NMR (400 MHz, DMSO d₆) δ (ppm)=2.12 (s, 3H), 2.22 (s, 3H), 5.38 (s, 2H), 7.15 (t, 1H), 7.71 (dd, 1H), 7.77 (ddd, 1H), 7.88-7.95 (m, 3H), 8.28 (s, 1H), 8.33-8.39 (m, 2H), 10.14 (s, 1H).

### Example 302

### N⁴-[1-(4-cyano-2-fluorobenzyl)-5-methyl-3-(trifluoromethyl)-1H-pyrazol-4-yl]quinoline-2,4-dicarboxamide

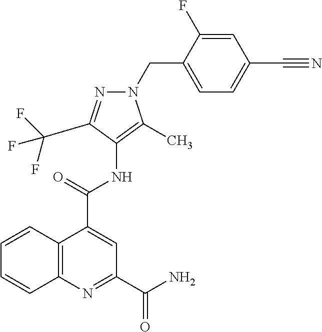 [SMILES: Cc1c(NC(=O)c2cc(C(N)=O)nc3ccccc23)c(C(F)(F)F)nn1Cc1ccc(C#N)cc1F]

In analogy to example 118), 178 mg (0.57 mmol) 4-{[4-amino-5-methyl-3-(trifluoromethyl)-1H-pyrazol-1-yl]methyl}-3-fluorobenzonitrile (intermediate 53C) and 102 mg (0.47 mmol) 2-carbamoylquinoline-4-carboxylic acid (intermediate 4A) were reacted to give after purification via preparative HPLC (method 3) 75 mg (31%) of the desired title compound.

¹H-NMR (400 MHz, DMSO d₆) δ (ppm)=2.33 (s, 3H), 5.63 (s, 2H), 7.33 (t, 1H), 7.78 (dd, 1H), 7.84 (ddd, 1H), 7.91-8.01 (m, 3H), 8.22 (ddd, 2H), 8.28 (s, 1H), 8.42 (d, 1H), 10.44 (s, 1H).

### Example 303

### 6-chloro-N⁴-[1-(4-cyano-2-fluorobenzyl)-5-methyl-3-(trifluoromethyl)-1H-pyrazol-4-yl]-7-fluoroquinoline-2,4-dicarboxamide

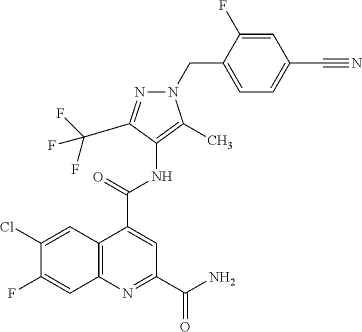 [SMILES: Cc1c(NC(=O)c2cc(C(N)=O)nc3cc(F)c(Cl)cc23)c(C(F)(F)F)nn1Cc1ccc(C#N)cc1F]

In analogy to example 118), 166 mg (0.53 mmol) 4-{[4-amino-5-methyl-3-(trifluoromethyl)-1H-pyrazol-1-yl]methyl}-3-fluorobenzonitrile (intermediate 53C) and 118 mg (0.44 mmol) 2-carbamoyl-6-chloro-7-fluoroquinoline-4-carboxylic acid (intermediate 32A) were reacted to give after purification via preparative HPLC (method 4) 52 mg (21%) of the desired title compound.

¹H-NMR (400 MHz, DMSO d₆) δ (ppm)=2.32 (s, 3H), 5.63 (s, 2H), 7.33 (t, 1H), 7.78 (dd, 1H), 7.97 (dd, 1H), 8.02 (d, 1H), 8.15 (d, 1H), 8.38 (s, 1H), 8.42 (br. s., 1H), 8.44 (d, 1H), 10.57 (s, 1H).

### Example 304

### N⁴-[1-(4-cyano-2-fluorobenzyl)-5-methyl-3-(trifluoromethyl)-1H-pyrazol-4-yl]-6,7-difluoroquinoline-2,4-dicarboxamide

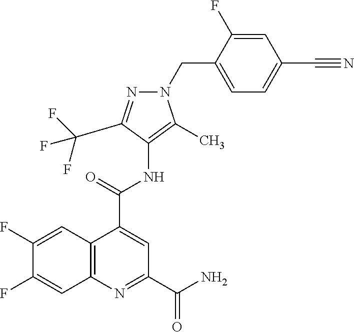 [SMILES: Cc1c(NC(=O)c2cc(C(N)=O)nc3cc(F)c(F)cc23)c(C(F)(F)F)nn1Cc1ccc(C#N)cc1F]

In analogy to example 118), 166 mg (0.53 mmol) 4-{[4-amino-5-methyl-3-(trifluoromethyl)-1H-pyrazol-1-yl]methyl}-3-fluorobenzonitrile (intermediate 53C) and 111 mg (0.44 mmol) 2-carbamoyl-6,7-difluoroquinoline-4-carboxylic acid (intermediate 3A) were reacted to give after purification via preparative HPLC (method 3) 107 mg (43%) of the desired title compound.

¹H-NMR (400 MHz, DMSO d₆) δ (ppm)=2.32 (s, 3H), 5.63 (s, 2H), 7.33 (t, 1H), 7.78 (dd, 1H), 7.97 (dd, 1H), 8.00 (s, 1H), 8.12-8.23 (m, 2H), 8.37-8.42 (m, 2H), 10.56 (s, 1H).

### Example 305

### 6-bromo-N⁴-[1-(4-cyano-2-fluorobenzyl)-5-methyl-3-(trifluoromethyl)-1H-pyrazol-4-yl]quinoline-2,4-dicarboxamide

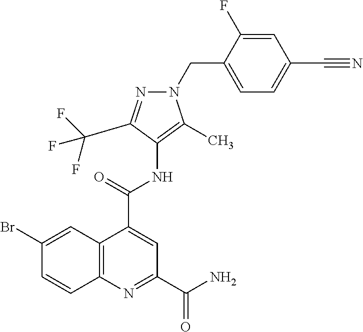 [SMILES: Cc1c(NC(=O)c2cc(C(N)=O)nc3ccc(Br)cc23)c(C(F)(F)F)nn1Cc1ccc(C#N)cc1F]

In analogy to example 118), 121 mg (0.41 mmol) 4-{[4-amino-5-methyl-3-(trifluoromethyl)-1H-pyrazol-1-yl]methyl}-3-fluorobenzonitrile (intermediate 53C) and 100 mg (0.34 mmol) 6-bromo-2-carbamoylquinoline-4-carboxylic acid (intermediate 2A) were reacted to give after purification via preparative HPLC (method 3) 71 mg (35%) of the desired title compound.

¹H-NMR (400 MHz, DMSO d₆) δ (ppm)=2.31 (s, 3H), 5.61 (s, 2H), 7.32 (t, 1H), 7.77 (dd, 1H), 7.91-7.98 (m, 2H), 8.08 (dd, 1H), 8.15 (d, 1H), 8.35 (s, 1H), 8.38-8.44 (m, 2H), 10.50 (s, 1H).

### Example 306

### N⁴-[1-(4-cyano-2-fluorobenzyl)-5-methyl-3-(trifluoromethyl)-1H-pyrazol-4-yl]-5-fluoroquinoline-2,4-dicarboxamide

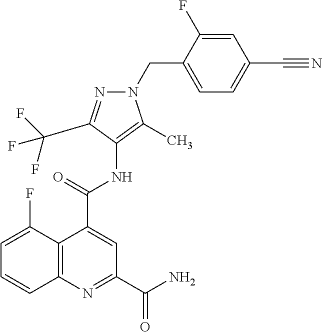 [SMILES: Cc1c(NC(=O)c2cc(C(N)=O)nc3cccc(F)c23)c(C(F)(F)F)nn1Cc1ccc(C#N)cc1F]

In analogy to example 118), 115 mg (0.38 mmol) 4-{[4-amino-5-methyl-3-(trifluoromethyl)-1H-pyrazol-1-yl]methyl}-3-fluorobenzonitrile (intermediate 53C) and 75 mg (0.32 mmol) 2-carbamoyl-5-fluoroquinoline-4-carboxylic acid (intermediate 39A) were reacted to give after purification via preparative HPLC (method 3) 65 mg (38%) of the desired title compound.

¹H-NMR (400 MHz, DMSO d₆) δ (ppm)=2.33 (s, 3H), 5.63 (s, 2H), 7.32 (t, 1H), 7.72-7.88 (m, 2H), 7.88-8.04 (m, 3H), 8.22-8.37 (m, 2H), 8.41 (br. s., 1H), 10.50 (s, 1H).

### Example 307

### 6-chloro-N⁴-[3-cyano-1-(4-fluorobenzyl)-5-methyl-1H-pyrazol-4-yl]-7-fluoroquinoline-2,4-dicarboxamide

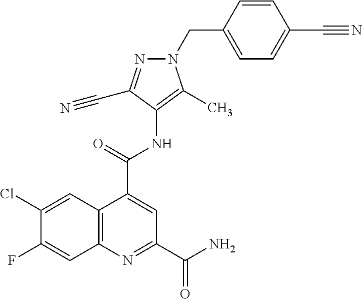 [SMILES: Cc1c(NC(=O)c2cc(C(N)=O)nc3cc(F)c(Cl)cc23)c(C#N)nn1Cc1ccc(C#N)cc1]

In analogy to example 118), 100 mg (0.41 mmol) 4-amino-1-(4-fluorobenzyl)-5-methyl-1H-pyrazole-3-carbonitrile (intermediate 54C) and 92 mg (0.34 mmol) 2-carbamoyl-6-chloro-7-fluoroquinoline-4-carboxylic acid (intermediate 32A) were reacted to give after purification via preparative HPLC (method 3) 71 mg (38%) of the desired title compound.

¹H-NMR (400 MHz, DMSO d₆) δ (ppm)=2.31 (s, 3H), 5.48 (s, 2H), 7.18-7.29 (m, 2H), 7.30-7.40 (m, 2H), 8.02 (s, 1H), 8.15 (d, 1H), 8.35-8.49 (m, 2H), 8.57 (d, 1H), 10.86 (s, 1H).

### Example 308

### N⁴-[3-cyano-1-(4-fluorobenzyl)-5-methyl-1H-pyrazol-4-yl]quinoline-2,4-dicarboxamide

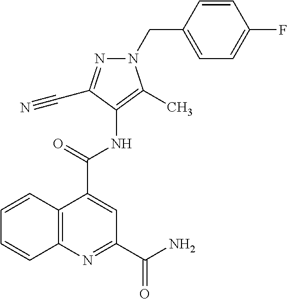 [SMILES: Cc1c(NC(=O)c2cc(C(N)=O)nc3ccccc23)c(C#N)nn1Cc1ccc(F)cc1]

In analogy to example 118), 100 mg (0.41 mmol) 4-amino-1-(4-fluorobenzyl)-5-methyl-1H-pyrazole-3-carbonitrile (intermediate 54C) and 74 mg (0.34 mmol) 2-carbamoylquinoline-4-carboxylic acid (intermediate 4A) were reacted to give after purification via preparative HPLC (method 3) 23 mg (15%) of the desired title compound.

¹H-NMR (400 MHz, DMSO d₆) δ (ppm)=2.31 (s, 3H), 5.49 (s, 2H), 7.25 (t, 2H), 7.35 (dd, 2H), 7.84 (d, 1H), 7.89-8.03 (m, 2H), 8.23 (d, 1H), 8.30 (d, 1H), 8.34 (s, 1H), 8.42 (br. s., 1H), 10.73 (s, 1H).

### Example 309

### 6-bromo-N-[1-(4-cyanobenzyl)-3,5-diethyl-1H-pyrazol-4-yl]-2-(trifluoromethyl)quinoline-4-carboxamide

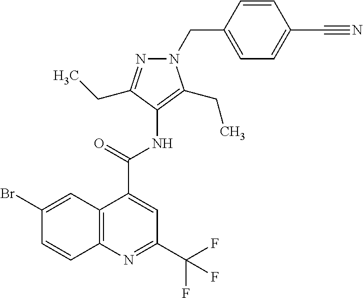 [SMILES: CCc1nn(Cc2ccc(C#N)cc2)c(CC)c1NC(=O)c1cc(C(F)(F)F)nc2ccc(Br)cc12]

In analogy to example 118), 95 mg (0.36 mmol) 4-[(4-amino-3,5-diethyl-1H-pyrazol-1-yl)methyl]benzonitrile (intermediate 55C) and 100 mg (0.31 mmol) 6-bromo-2-(trifluoromethyl)quinoline-4-carboxylic acid (intermediate 1A) were reacted to give after purification via preparative HPLC (method 4) 129 mg (71%) of the desired title compound.

¹H-NMR (300 MHz, DMSO d₆) δ (ppm)=1.02 (t, 3H), 1.19 (t, 3H), 2.52-2.66 (m, 4H), 5.42 (s, 2H), 7.30 (d, 2H), 7.85 (d, 2H), 8.15 (dd, 1H), 8.21-8.27 (m, 2H), 8.45 (d, 1H), 10.14 (s, 1H).

### Example 310

### N⁴-[1-(4-cyanobenzyl)-3,5-diethyl-1H-pyrazol-4-yl]quinoline-2,4-dicarboxamide

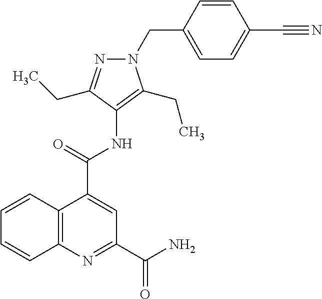 [SMILES: CCc1nn(Cc2ccc(C#N)cc2)c(CC)c1NC(=O)c1cc(C(N)=O)nc2ccccc12]

In analogy to example 118), 141 mg (0.56 mmol) 4-[(4-amino-3,5-diethyl-1H-pyrazol-1-yl)methyl]benzonitrile (intermediate 55C) and 100 mg (0.46 mmol) 2-carbamoylquinoline-4-carboxylic acid (intermediate 4A) were reacted to give after purification via preparative HPLC (method 4) 112 mg (51%) of the desired title compound.

¹H-NMR (300 MHz, DMSO d₆) δ (ppm)=1.03 (t, 3H), 1.19 (t, 3H), 2.51-2.66 (m, 4H), 5.41 (s, 2H), 7.31 (d, 2H), 7.78-7.98 (m, 5H), 8.17-8.28 (m, 2H), 8.40 (s, 1H), 10.05 (s, 1H).

### Example 311

### 6-chloro-N⁴-[1-(4-cyanobenzyl)-3,5-diethyl-1H-pyrazol-4-yl]-7-fluoroquinoline-2,4-dicarboxamide

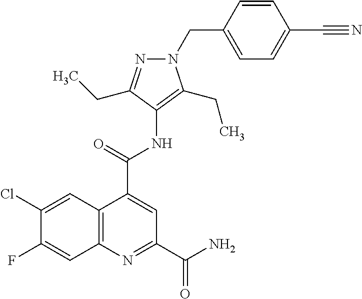 [SMILES: CCc1nn(Cc2ccc(C#N)cc2)c(CC)c1NC(=O)c1cc(C(N)=O)nc2cc(F)c(Cl)cc12]

In analogy to example 118), 113 mg (0.45 mmol) 4-[(4-amino-3,5-diethyl-1H-pyrazol-1-yl)methyl]benzonitrile (intermediate 55C) and 100 mg (0.37 mmol) 2-carbamoyl-6-chloro-7-fluoroquinoline-4-carboxylic acid (intermediate 32A) were reacted to give after purification via preparative HPLC (method 4) 78 mg (39%) of the desired title compound.

¹H-NMR (300 MHz, DMSO d₆) δ (ppm)=1.01 (t, 3H), 1.18 (t, 3H), 2.52-2.66 (m, 4H), 5.41 (s, 2H), 7.31 (d, 2H), 7.85 (d, 2H), 8.01 (s, 1H), 8.13 (d, 1H), 8.33 (s, 1H), 8.37-8.47 (m, 2H), 10.18 (s, 1H).

### Example 312

### N⁴-[1-(4-cyanobenzyl)-3,5-diethyl-1H-pyrazol-4-yl]-7-fluoroquinoline-2,4-dicarboxamide

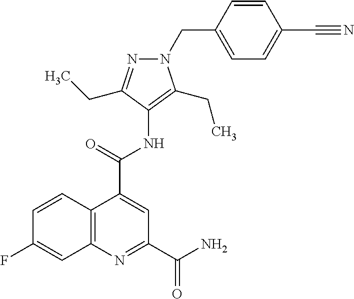 [SMILES: CCc1nn(Cc2ccc(C#N)cc2)c(CC)c1NC(=O)c1cc(C(N)=O)nc2cc(F)ccc12]

In analogy to example 118), 98 mg (0.38 mmol) 4-[(4-amino-3,5-diethyl-1H-pyrazol-1-yl)methyl]benzonitrile (intermediate 55C) and 75 mg (0.32 mmol) 2-carbamoyl-7-fluoroquinoline-4-carboxylic acid (intermediate 37A) were reacted to give after purification via preparative HPLC (method 4) 109 mg (69%) of the desired title compound.

¹H-NMR (300 MHz, DMSO d₆) δ (ppm)=1.02 (t, 3H), 1.18 (t, 3H), 2.50-2.66 (m, 4H), 5.41 (s, 2H), 7.31 (d, 2H), 7.75-7.87 (m, 3H), 7.91 (dd, 1H), 7.97 (s, 1H), 8.24 (s, 1H), 8.30 (dd, 1H), 8.40 (s, 1H), 10.10 (s, 1H).

### Example 313

### 7-chloro-N⁴-[1-(4-cyanobenzyl)-3,5-diethyl-1H-pyrazol-4-yl]quinoline-2,4-dicarboxamide

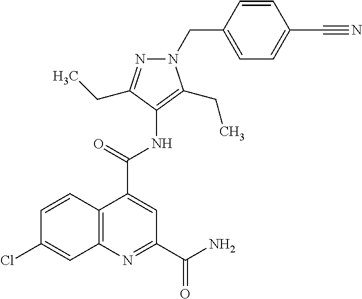 [SMILES: CCc1nn(Cc2ccc(C#N)cc2)c(CC)c1NC(=O)c1cc(C(N)=O)nc2cc(Cl)ccc12]

In analogy to example 118), 122 mg (0.48 mmol) 4-[(4-amino-3,5-diethyl-1H-pyrazol-1-yl)methyl]benzonitrile (intermediate 55C) and 100 mg (0.40 mmol) 2-carbamoyl-7-chloroquinoline-4-carboxylic acid (intermediate 48A) were reacted to give after purification via preparative HPLC (method 4) 96 mg (49%) of the desired title compound.

¹H-NMR (300 MHz, DMSO d₆) δ (ppm)=1.01 (t, 3H), 1.18 (t, 3H), 2.51-2.67 (m, 4H), 5.41 (s, 2H), 7.31 (d, 2H), 7.81-7.93 (m, 3H), 7.99 (br. s., 1H), 8.21-8.29 (m, 2H), 8.41 (s, 1H), 10.10 (s, 1H).

### Example 314

### 6-chloro-N⁴-[1-(4-cyanobenzyl)-5-ethyl-3-(trifluoromethyl)-1H-pyrazol-4-yl]-7-fluoroquinoline-2,4-dicarboxamide

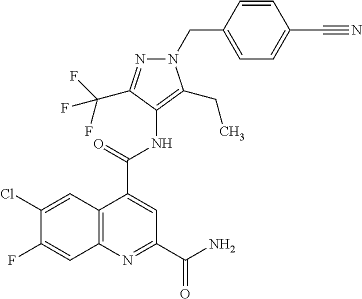 [SMILES: CCc1c(NC(=O)c2cc(C(N)=O)nc3cc(F)c(Cl)cc23)c(C(F)(F)F)nn1Cc1ccc(C#N)cc1]

In analogy to example 118), 105 mg (0.36 mmol) 4-{[4-amino-5-ethyl-3-(trifluoromethyl)-1H-pyrazol-1-yl]methyl}benzonitrile (intermediate 56C) and 80 mg (0.30 mmol) 2-carbamoyl-6-chloro-7-fluoroquinoline-4-carboxylic acid (intermediate 32A) were reacted to give after purification via preparative HPLC (method 3) 61 mg (35%) of the desired title compound.

¹H-NMR (400 MHz, DMSO d₆) δ (ppm)=1.05 (t, 3H), 2.73 (q, 2H), 5.62 (s, 2H), 7.38 (d, 2H), 7.89 (d, 2H), 8.00 (s, 1H), 8.14 (d, 1H), 8.32 (s, 1H), 8.35-8.44 (m, 2H), 10.52 (s, 1H).

### Example 315

### N⁴-[1-(4-cyanobenzyl)-5-ethyl-3-(trifluoromethyl)-1H-pyrazol-4-yl]-7-fluoroquinoline-2,4-dicarboxamide

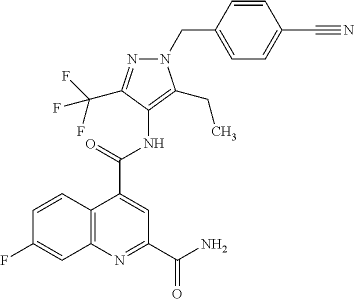 [SMILES: CCc1c(NC(=O)c2cc(C(N)=O)nc3cc(F)ccc23)c(C(F)(F)F)nn1Cc1ccc(C#N)cc1]

In analogy to example 118), 121 mg (0.41 mmol) 4-{[4-amino-5-ethyl-3-(trifluoromethyl)-1H-pyrazol-1-yl]methyl}benzonitrile (intermediate 56C) and 80 mg (0.34 mmol) 2-carbamoyl-7-fluoroquinoline-4-carboxylic acid (intermediate 37A) were reacted to give after purification via preparative HPLC (method 3) 66 mg (36%) of the desired title compound.

¹H-NMR (400 MHz, DMSO d₆) δ (ppm)=1.05 (t, 3H), 2.72 (q, 2H), 5.62 (s, 2H), 7.38 (d, 2H), 7.74-7.85 (m, 1H), 7.86-7.99 (m, 4H), 8.22-8.30 (m, 2H), 8.38 (br. s., 1H), 10.44 (s, 1H).

### Example 316

### N⁴-[1-(4-cyanobenzyl)-5-ethyl-3-(trifluoromethyl)-1H-pyrazol-4-yl]-5-fluoroquinoline-2,4-dicarboxamide

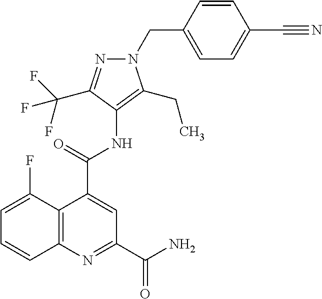 [SMILES: CCc1c(NC(=O)c2cc(C(N)=O)nc3cccc(F)c23)c(C(F)(F)F)nn1Cc1ccc(C#N)cc1]

In analogy to example 118), 121 mg (0.41 mmol) 4-{[4-amino-5-ethyl-3-(trifluoromethyl)-1H-pyrazol-1-yl]methyl}benzonitrile (intermediate 56C) and 80 mg (0.34 mmol) 2-carbamoyl-5-fluoroquinoline-4-carboxylic acid (intermediate 39A) were reacted to give after purification via preparative HPLC (method 3) 78 mg (36%) of the desired title compound.

¹H-NMR (400 MHz, DMSO d₆) δ (ppm)=1.05 (t, 3H), 2.72 (q, 2H), 5.62 (s, 2H), 7.38 (d, 2H), 7.75-7.85 (m, 1H), 7.87-7.98 (m, 4H), 8.22-8.29 (m, 2H), 8.38 (s, 1H), 10.44 (s, 1H).

### Example 317

### N⁴-[1-(4-cyanobenzyl)-5-isopropyl-3-(trifluoromethyl)-1H-pyrazol-4-yl]quinoline-2,4-dicarboxamide

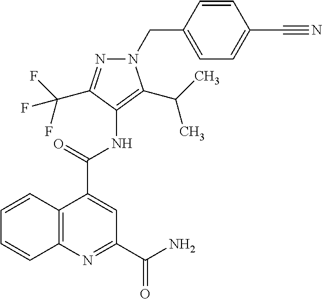 [SMILES: CC(C)c1c(NC(=O)c2cc(C(N)=O)nc3ccccc23)c(C(F)(F)F)nn1Cc1ccc(C#N)cc1]

In analogy to example 118), 232 mg (0.41 mmol, purity about 80%) 4-{[4-amino-5-isopropyl-3-(trifluoromethyl)-1H-pyrazol-1-yl]methyl}benzonitrile (intermediate 57C) and 203 mg (0.75 mmol) 2-carbamoylquinoline-4-carboxylic acid (intermediate 4A) were reacted to give after purification via two subsequent preparative HPLC (method 4) 27 mg (6.7%) of the desired title compound.

¹H-NMR (500 MHz, DMSO d₆) δ (ppm)=1.22 (d, 6H), 3.24 (spt, 1H), 5.64 (s, 2H), 7.36 (d, 2H), 7.82 (ddd, 1H), 7.87-7.97 (m, 4H), 8.18-8.24 (m, 3H), 8.38-8.42 (m, 1H), 10.36 (s, 1H).

### Example 318

### N⁴-[1-(4-cyanobenzyl)-5-isopropyl-3-(trifluoromethyl)-1H-pyrazol-4-yl]-7-fluoroquinoline-2,4-dicarboxamide

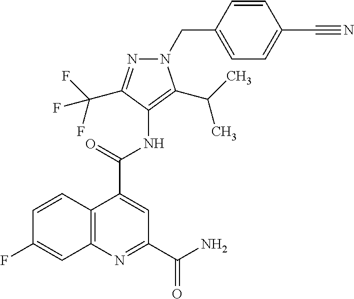 [SMILES: CC(C)c1c(NC(=O)c2cc(C(N)=O)nc3cc(F)ccc23)c(C(F)(F)F)nn1Cc1ccc(C#N)cc1]

In analogy to example 118), 148 mg (0.38 mmol, purity about 80%) 4-{[4-amino-5-isopropyl-3-(trifluoromethyl)-1H-pyrazol-1-yl]methyl}benzonitrile (intermediate 57C) and 75 mg (0.332 mmol) 2-carbamoyl-7-fluoroquinoline-4-carboxylic acid (intermediate 37A) were reacted to give after purification via preparative HPLC (method 5d) 12 mg (6.7%) of the desired title compound.

¹H-NMR (400 MHz, DMSO d₆) δ (ppm)=1.20 (d, 6H), 3.24 (spt, 1H), 5.64 (s, 2H), 7.36 (d, 2H), 7.78-7.85 (m, 1H), 7.89 (d, 3H), 7.96 (d, 1H), 8.22 (s, 1H), 8.27 (dd, 1H), 8.39 (d, 1H), 10.41 (s, 1H).

### Example 319

### N⁴-[1-(4-cyanobenzyl)-3-isopropyl-5-(trifluoromethyl)-1H-pyrazol-4-yl]quinoline-2,4-dicarboxamide

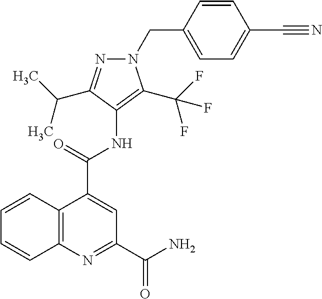 [SMILES: CC(C)c1nn(Cc2ccc(C#N)cc2)c(C(F)(F)F)c1NC(=O)c1cc(C(N)=O)nc2ccccc12]

In analogy to example 118), 175 mg (0.57 mmol) 4-{[4-amino-3-isopropyl-5-(trifluoromethyl)-1H-pyrazol-1-yl]methyl}benzonitrile (intermediate 58C) and 102 mg (0.47 mmol) 2-carbamoylquinoline-4-carboxylic acid (intermediate 4A) were reacted to give after purification via a preparative HPLC (method 3) and finally via a Biotage chromatography system (10 g snap KP-Sil column, hexane/0-100% ethyl acetate, then ethyl acetate/0-50% methanol) 20 mg (7.6%) of the desired title compound.

¹H-NMR (400 MHz, DMSO ds) δ (ppm)=1.30 (d, 6H), 3.09 (spt, 1H), 5.63 (s, 2H), 7.34 (d, 2H), 7.79-8.00 (m, 5H), 8.17-8.29 (m, 3H), 8.40-8.47 (m, 1H), 10.53 (s, 1H).

### Example 320

### N⁴-[1-(4-cyanobenzyl)-3-isopropyl-5-(trifluoromethyl)-1H-pyrazol-4-yl]-7-fluoroquinoline-2,4-dicarboxamide

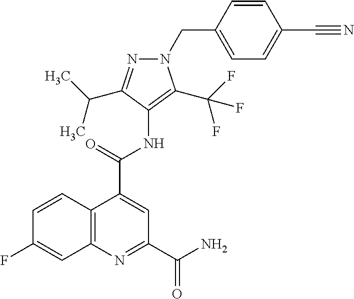 [SMILES: CC(C)c1nn(Cc2ccc(C#N)cc2)c(C(F)(F)F)c1NC(=O)c1cc(C(N)=O)nc2cc(F)ccc12]

In analogy to example 118), 175 mg (0.57 mmol) 4-{[4-amino-3-isopropyl-5-(trifluoromethyl)-1H-pyrazol-1-yl]methyl}benzonitrile (intermediate 58C) and 111 mg (0.47 mmol) 2-carbamoyl-7-fluoroquinoline-4-carboxylic acid (intermediate 37A) were reacted to give after purification via preparative HPLC (method 5d) 27 mg (9.9%) of the desired title compound.

¹H-NMR (400 MHz, DMSO d₆) δ (ppm)=1.29 (d, 6H), 3.08 (spt, 1H), 5.63 (s, 2H), 7.34 (d, 2H), 7.83 (ddd, 1H), 7.89 (d, 2H), 7.94 (dd, 1H), 8.00 (d, 1H), 8.24 (s, 1H), 8.28 (dd, 1H), 8.42 (s, 1H), 10.58 (s, 1H).

### Example 321

### N⁴-[1-(4-cyanobenzyl)-3-isopropyl-5-methyl-1H-pyrazol-4-yl]-7-fluoroquinoline-2,4-dicarboxamide

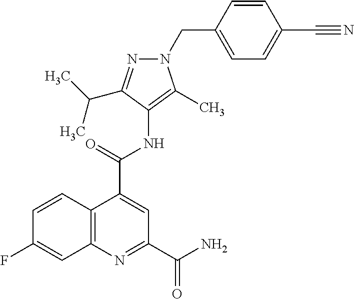 [SMILES: Cc1c(NC(=O)c2cc(C(N)=O)nc3cc(F)ccc23)c(C(C)C)nn1Cc1ccc(C#N)cc1]

In analogy to example 118), 163 mg (0.64 mmol) 4-[(4-amino-3-isopropyl-5-methyl-1H-pyrazol-1-yl)methyl]benzonitrile (intermediate 59C) together with a small amount of 4-[(4-amino-5-isopropyl-3-methyl-1H-pyrazol-1-yl)methyl]benzonitrile (intermediate 60C) and 125 mg (0.53 mmol) 2-carbamoyl-7-fluoroquinoline-4-carboxylic acid (intermediate 37A) were reacted to give after purification via preparative HPLC (method 5d) 80 mg (30%) of the desired title compound.

¹H-NMR (400 MHz, DMSO d₆) δ (ppm)=1.23 (d, 6H), 2.13 (s, 3H), 2.98 (spt, 1H), 5.41 (s, 2H), 7.29 (d, 2H), 7.77 (ddd, 1H), 7.84 (d, 2H), 7.91 (dd, 1H), 7.94 (d, 1H), 8.25 (s, 1H), 8.31 (dd, 1H), 8.37 (d, 1H), 10.07 (s, 1H).

### Example 322 and Example 323

### N⁴-[1-(4-cyanobenzyl)-3-isopropyl-5-methyl-1H-pyrazol-4-yl]quinoline-2,4-dicarboxamide and N⁴-[1-(4-cyanobenzyl)-5-isopropyl-3-methyl-1H-pyrazol-4-yl]quinoline-2,4-dicarboxamide

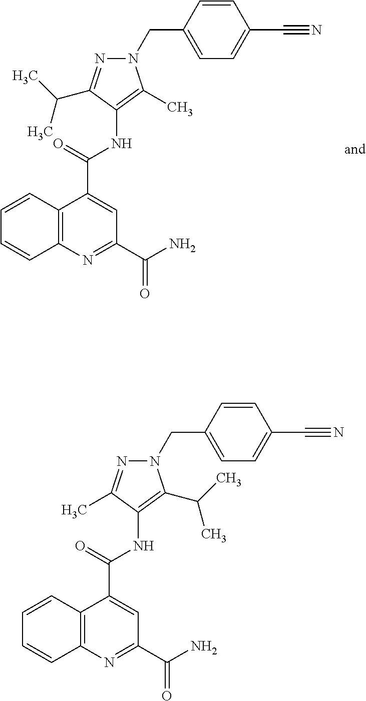 [SMILES: Cc1c(NC(=O)c2cc(C(N)=O)nc3ccccc23)c(C(C)C)nn1Cc1ccc(C#N)cc1 ; Cc1nn(Cc2ccc(C#N)cc2)c(C(C)C)c1NC(=O)c1cc(C(N)=O)nc2ccccc12]

In analogy to example 118), 176 mg (0.69 mmol) 4-[(4-amino-3-isopropyl-5-methyl-1H-pyrazol-1-yl)methyl]benzonitrile (intermediate 59C) together with a small amount of 4-[(4-amino-5-isopropyl-3-methyl-1H-pyrazol-1-yl)methyl]benzonitrile (intermediate 60C) and 125 mg (0.58 mmol) 2-carbamoylquinoline-4-carboxylic acid (intermediate 4A) were reacted to give after purification via preparative HPLC (method 5d) 99 mg (36%) of N⁴-[1-(4-cyanobenzyl)-3-isopropyl-5-methyl-1H-pyrazol-4-yl]quinoline-2,4-dicarboxamide and 3.4 mg (1.2%) of its regioisomer N⁴-[1-(4-cyanobenzyl)-5-isopropyl-3-methyl-1H-pyrazol-4-yl]quinoline-2,4-dicarboxamide as the desired title compounds.

NMR of N⁴-[1-(4-cyanobenzyl)-3-isopropyl-5-methyl-1H-pyrazol-4-yl]quinoline-2,4-dicarboxamide as example 322:

¹H-NMR (400 MHz, DMSO d₆) δ (ppm)=1.25 (d, 6H), 2.16 (s, 3H), 3.01 (spt, 1H), 5.43 (s, 2H), 7.31 (d, 2H), 7.80-7.88 (m, 3H), 7.90-7.97 (m, 2H), 8.23 (t, 2H), 8.27 (s, 1H), 8.41 (s, 1H), 10.05 (s, 1H).

NMR of N⁴-[1-(4-cyanobenzyl)-5-isopropyl-3-methyl-1H-pyrazol-4-yl]quinoline-2,4-dicarboxamide as example 323:

¹H-NMR (400 MHz, CDCl₃) δ (ppm)=1.29 (d, 6H), 2.31 (s, 3H), 3.02 (spt, 1H), 5.38 (s, 2H), 5.74 (d, 1H), 7.22 (d, 2H), 7.32 (s, 1H), 7.66 (d, 2H), 7.78 (ddd, 1H), 7.89 (ddd, 1H), 8.09 (d, 1H), 8.23 (d, 1H), 8.46-8.53 (m, 2H).

### Example 324

### 6-chloro-N⁴-[1-(4-cyanobenzyl)-3-isopropyl-5-methyl-1H-pyrazol-4-yl]-7-fluoroquinoline-2,4-dicarboxamide

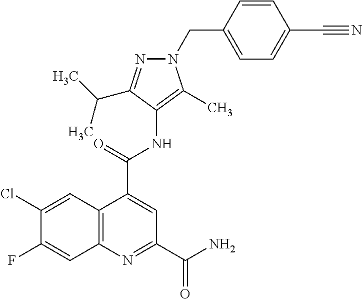 [SMILES: Cc1c(NC(=O)c2cc(C(N)=O)nc3cc(F)c(Cl)cc23)c(C(C)C)nn1Cc1ccc(C#N)cc1]

In analogy to example 118), 142 mg (0.56 mmol) 4-[(4-amino-3-isopropyl-5-methyl-1H-pyrazol-1-yl)methyl]benzonitrile (intermediate 59C) and 125 mg (0.47 mmol) 2-carbamoyl-6-chloro-7-fluoroquinoline-4-carboxylic acid (intermediate 32A) were reacted to give a solid during the work-up procedure, yielding 45 mg (18%) of the desired title compound.

¹H-NMR (400 MHz, DMSO d₆) δ (ppm)=1.24 (d, 6H), 2.14 (s, 3H), 2.99 (spt, 1H), 5.42 (s, 2H), 7.31 (d, 2H), 7.86 (d, 2H), 8.01 (br. s., 1H), 8.14 (d, 1H), 8.37 (s, 1H), 8.41 (s, 1H), 8.48 (d, 1H), 10.18 (br. s., 1H).

### Example 325 and Example 326

N⁴-[1-(4-cyanobenzyl)-3-ethyl-5-methyl-1H-pyrazol-4-yl]quinoline-2,4-dicarboxamide and N⁴-[1-(4-cyanobenzyl)-5-ethyl-3-methyl-1H-pyrazol-4-yl]quinoline-2,4-dicarboxamide

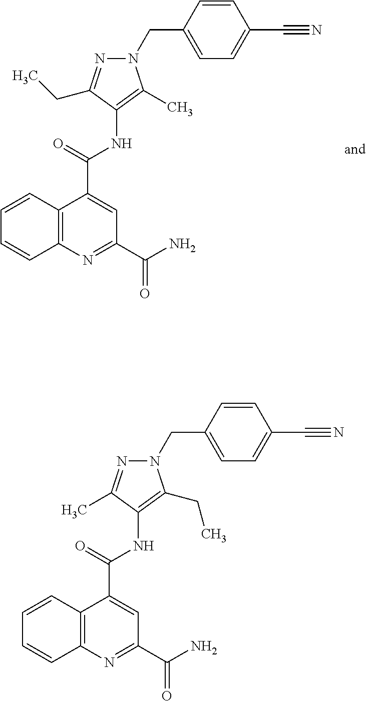 [SMILES: CCc1nn(Cc2ccc(C#N)cc2)c(C)c1NC(=O)c1cc(C(N)=O)nc2ccccc12 ; CCc1c(NC(=O)c2cc(C(N)=O)nc3ccccc23)c(C)nn1Cc1ccc(C#N)cc1]

In analogy to example 118), 267 mg (1.10 mmol) of a mixture of 4-[(4-amino-3-ethyl-5-methyl-1H-pyrazol-1-yl)methyl]benzonitrile and 4-[(4-amino-5-ethyl-3-methyl-1H-pyrazol-1-yl)methyl]benzonitrile (intermediate 61C and 62C) and 200 mg (0.93 mmol) 2-carbamoylquinoline-4-carboxylic acid (intermediate 4A) were reacted to give after a first purification via a Biotage chromatography system (25 g snap KP-Sil column, ethyl acetate/0-30% methanol) and finally a purification/separation via preparative HPLC (method 7) 72 mg (16%) of N⁴-[1-(4-cyanobenzyl)-3-ethyl-5-methyl-1H-pyrazol-4-yl]quinoline-2,4-dicarboxamide and 65 mg (14%) of its regioisomer N⁴-[1-(4-cyanobenzyl)-5-ethyl-3-methy-1H-pyrazol-4-yl]quinoline-2,4-dicarboxamide as the desired title compounds.

NMR of N⁴-[1-(4-cyanobenzyl)-3-ethyl-5-methyl-1H-pyrazol-4-yl]quinoline-2,4-dicarboxamide as example 325:

¹H-NMR (400 MHz, DMSO d₆) δ (ppm)=1.18 (t, 3H), 2.16 (s, 3H), 2.56 (q, 2H), 5.40 (s, 2H), 7.31 (d, 2H), 7.78-7.86 (m, 3H), 7.87-7.90 (m, 1H), 7.91-7.96 (m, 1H), 8.20 (d, 1H), 8.23 (d, 1H), 8.26 (s, 1H), 8.37 (s, 1H), 10.06 (s, 1H). NMR of N⁴-[1-(4-cyanobenzyl)-5-ethyl-3-methyl-1H-pyrazol-4-yl]quinoline-2,4-dicarboxamide as example 326:

¹H-NMR (400 MHz, DMSO d₆) δ (ppm)=1.04 (t, 3H), 2.16 (s, 3H), 2.64 (q, 2H), 5.40 (s, 2H), 7.33 (d, 2H), 7.75-7.87 (m, 3H), 7.90 (br. s., 1H), 7.92-7.97 (m, 1H), 8.20-8.26 (m, 2H), 8.26 (s, 1H), 8.39 (s, 1H), 10.06 (s, 1H).

### Example 327 and Example 328

### N⁴-[1-(4-cyanobenzyl)-3-ethyl-5-methyl-1H-pyrazol-4-yl]-7-fluoroquinoline-2,4-dicarboxamide and N⁴-[1-(4-cyanobenzyl)-5-ethyl-3-methyl-1H-pyrazol-4-yl]-7-fluoroquinoline-2,4-dicarboxamide

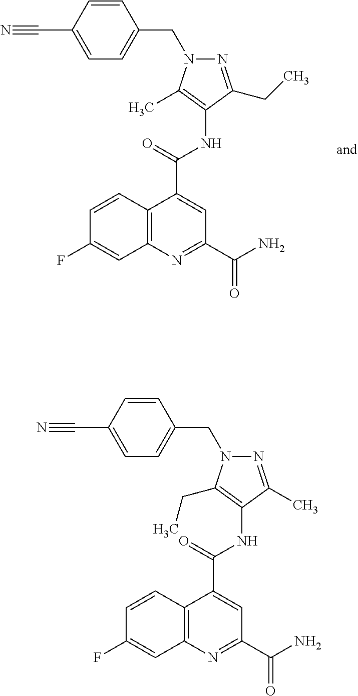 [SMILES: CCc1nn(Cc2ccc(C#N)cc2)c(C)c1NC(=O)c1cc(C(N)=O)nc2cc(F)ccc12 ; CCc1c(NC(=O)c2cc(C(N)=O)nc3cc(F)ccc23)c(C)nn1Cc1ccc(C#N)cc1]

In analogy to example 118), 247 mg (1.03 mmol) of a mixture of 4-[(4-amino-3-ethyl-5-methyl-1H-pyrazol-1-yl)methyl]benzonitrile and 4-[(4-amino-5-ethyl-3-methyl-1H-pyrazol-1-yl)methyl]benzonitrile (intermediate 61C and 62C) and 200 mg (0.85 mmol) 2-carbamoyl-7-fluoroquinoline-4-carboxylic acid (intermediate 37A) were reacted to give after a first purification via a Biotage chromatography system (25 g snap KP-Sil column, ethyl acetate/0-30% methanol) and finally a purification/separation via preparative HPLC (method 8) 39 mg (9.0%) of N⁴-[1-(4-cyanobenzyl)-3-ethyl-5-methyl-1H-pyrazol-4-yl]-7-fluoroquinoline-2,4-dicarboxamide and 11 mg (2.9%) of its regioisomer N⁴-[1-(4-cyanobenzyl)-5-ethyl-3-methyl-1H-pyrazol-4-yl]-7-fluoroquinoline-2,4-dicarboxamide as the desired title compounds.

### NMR of N⁴-[1-(4-cyanobenzyl)-3-ethyl-5-methyl-1H-pyrazol-4-yl]-7-fluoroquinoline-2,4-dicarboxamide as example 327

¹H-NMR (400 MHz, DMSO d₆) δ (ppm)=1.19 (t, 3H), 2.17 (s, 3H), 2.57 (q, 2H), 5.41 (s, 2H), 7.30-7.35 (m, 2H), 7.76-7.82 (m, 1H), 7.86 (d, 2H), 7.93 (dd, 1H), 7.97 (br. s., 1H), 8.28 (s, 1H), 8.31-8.37 (m, 1H), 8.40 (br. s., 1H), 10.13 (s, 1H).

### NMR of N⁴-[1-(4-cyanobenzyl)-5-ethyl-3-methyl-1H-pyrazol-4-yl]-7-fluoroquinoline-2,4-dicarboxamide as example 328

¹H-NMR (400 MHz, DMSO d₆) δ (ppm)=1.04 (t, 3H), 2.16 (s, 3H), 2.64 (q, 2H), 5.41 (s, 2H), 7.31-7.36 (m, 2H), 7.77-7.83 (m, 1H), 7.86 (d, 2H), 7.91-7.95 (m, 1H), 7.96-7.98 (m, 1H), 8.27 (s, 1H), 8.31-8.37 (m, 1H), 8.39-8.42 (m, 1H), 10.13 (s, 1H).

### Example 329

### (±)-N⁴-{1-[1-(4-cyanophenyl)ethyl]-3,5-dimethyl-1H-pyrazol-4-yl}quinoline-2,4-dicarboxamide

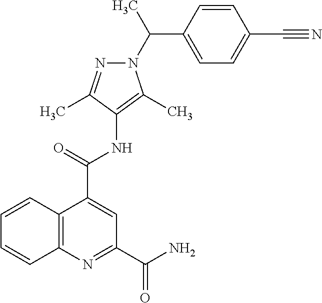 [SMILES: Cc1nn(C(C)c2ccc(C#N)cc2)c(C)c1NC(=O)c1cc(C(N)=O)nc2ccccc12]

In analogy to example 118), 133 mg (0.56 mmol) (±)-4-[1-(4-amino-3,5-dimethyl-1H-pyrazol-1-yl)ethyl]benzonitrile (intermediate 63C) and 100 mg (0.46 mmol) 2-carbamoylquinoline-4-carboxylic acid (intermediate 4A) were reacted to give after purification via preparative HPLC (method 4) 81 mg (40%) of the desired title compound.

¹H-NMR (400 MHz, DMSO d₆) δ (ppm)=1.79 (d, 3H), 2.13 (s, 3H), 2.18 (s, 3H), 5.70 (q, 1H), 7.40 (d, 2H), 7.77-7.84 (m, 3H), 7.88 (d, 1H), 7.90-7.95 (m, 1H), 8.20 (d, 1H), 8.24 (dd, 1H), 8.26 (s, 1H), 8.37 (d, 1H), 10.04 (s, 1H).

### Example 330

### (±)-6-chloro-N⁴-{1-[1-(4-cyanophenyl)ethyl]-3,5-dimethyl-1H-pyrazol-4-yl}-7-fluoroquinoline-2,4-dicarboxamide

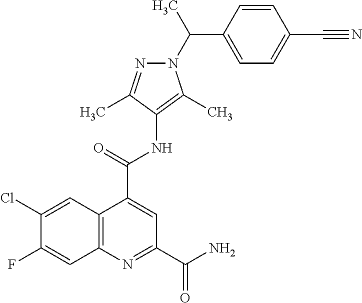 [SMILES: Cc1nn(C(C)c2ccc(C#N)cc2)c(C)c1NC(=O)c1cc(C(N)=O)nc2cc(F)c(Cl)cc12]

In analogy to example 118), 107 mg (0.45 mmol) (±)-4-[1-(4-amino-3,5-dimethyl-1H-pyrazol-1-yl)ethyl]benzonitrile (intermediate 63C) and 100 mg (0.37 mmol) 2-carbamoyl-6-chloro-7-fluoroquinoline-4-carboxylic acid (intermediate 32A) were reacted to give after purification via preparative HPLC (method 4) 66 mg (35%) of the desired title compound.

¹H-NMR (400 MHz, DMSO d₆) δ (ppm)=1.79 (d, 3H), 2.16 (s, 3H), 2.12 (s, 3H), 5.69 (q, 1H), 7.40 (d, 2H), 7.80-7.88 (m, 2H), 7.92-8.01 (m, 1H), 8.10 (d, 1H), 8.34-8.40 (m, 2H), 8.50 (d, 1H), 10.18 (s, 1H).

### Example 331

### (±)-N⁴-{1-[1-(4-cyanophenyl)ethyl]-3,5-dimethyl-1H-pyrazol-4-yl}-8-fluoroquinoline-2,4-dicarboxamide

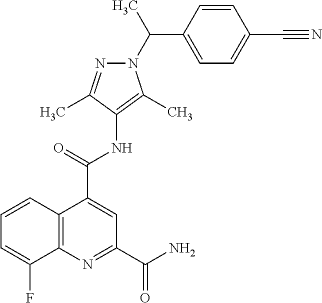 [SMILES: Cc1nn(C(C)c2ccc(C#N)cc2)c(C)c1NC(=O)c1cc(C(N)=O)nc2c(F)cccc12]

In analogy to example 118), 123 mg (0.51 mmol) (±)-4-[1-(4-amino-3,5-dimethyl-1H-pyrazol-1-yl)ethyl]benzonitrile (intermediate 63C) and 100 mg (0.43 mmol) 2-carbamoyl-8-fluoroquinoline-4-carboxylic acid (intermediate 47A) were reacted to give after purification via preparative HPLC (method 4) 80 mg (38%) of the desired title compound.

¹H-NMR (400 MHz, DMSO d₆) δ (ppm)=1.79 (d, 3H), 2.13 (s, 3H), 2.17 (s, 3H), 5.70 (q, 1H), 7.40 (d, 2H), 7.74-7.86 (m, 4H), 7.96 (s, 1H), 8.02-8.07 (m, 1H), 8.22 (s, 1H), 8.33 (s, 1H), 10.09 (s, 1H).

### Example 332

### (±)-N⁴-{1-[1-(4-cyanophenyl)ethyl]-3,5-dimethyl-1H-pyrazol-4-yl}-8-fluoroquinoline-2,4-dicarboxamide

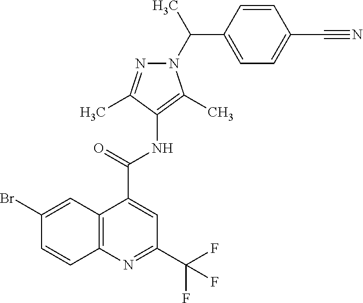 [SMILES: Cc1nn(C(C)c2ccc(C#N)cc2)c(C)c1NC(=O)c1cc(C(F)(F)F)nc2ccc(Br)cc12]

In analogy to example 118), 90 mg (0.36 mmol) (±)-4-[1-(4-amino-3,5-dimethyl-1H-pyrazol-1-yl)ethyl]benzonitrile (intermediate 63C) and 100 mg (0.31 mmol) 6-bromo-2-(trifluoromethyl)quinoline-4-carboxylic acid (intermediate 1A) were reacted to give after purification via preparative HPLC (method 4) 119 mg (68%) of the desired title compound.

¹H-NMR (400 MHz, DMSO d₆) δ (ppm)=1.79 (d, 3H), 2.13 (s, 3H), 2.18 (s, 3H), 5.70 (q, 1H), 7.39 (d, 2H), 7.80-7.85 (m, 2H), 8.11-8.16 (m, 1H), 8.20-8.24 (m, 1H), 8.27 (s, 1H), 8.49 (d, 1H), 10.14 (s, 1H).

### Example 333

### (±)-N⁴-{1-[1-(4-cyanophenyl)ethyl]-5-methyl-3-(trifluoromethyl)-1H-pyrazol-4-yl}quinoline-2,4-dicarboxamide

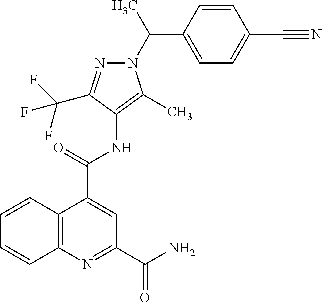 [SMILES: Cc1c(NC(=O)c2cc(C(N)=O)nc3ccccc23)c(C(F)(F)F)nn1C(C)c1ccc(C#N)cc1]

In analogy to example 118), 163 mg (0.56 mmol) (±)-4-{1-[4-amino-5-methyl-3-(trifluoromethyl)-1H-pyrazol-1-yl]ethyl}benzonitrile (intermediate 64C) and 100 mg (0.46 mmol) 2-carbamoylquinoline-4-carboxylic acid (intermediate 4A) were reacted to give after purification via a Biotage chromatography system (10 g snap KP-Sil column, hexane/10-70% ethyl acetate) 93 mg (39%) of the desired title compound.

¹H-NMR (400 MHz, DMSO d) δ (ppm)=1.85 (d, 3H), 2.22 (s, 3H), 5.96 (q, 1H), 7.43 (d, 2H), 7.81 (ddd, 1H), 7.85-7.91 (m, 3H), 7.91-7.96 (m, 1H), 8.16-8.22 (m, 2H), 8.25 (s, 1H), 8.36-8.40 (m, 1H), 10.37 (s, 1H).

### Example 334

### (±)-7-fluoro-N⁴-[5-methyl-1-(1-phenylethyl)-3-(trifluoromethyl)-1H-pyrazol-4-yl]quinoline-2,4-dicarboxamide

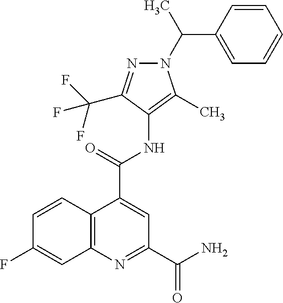 [SMILES: Cc1c(NC(=O)c2cc(C(N)=O)nc3cc(F)ccc23)c(C(F)(F)F)nn1C(C)c1ccccc1]

In analogy to example 118), 560 mg (2.08 mmol) (±)-5-methyl-1-(1-phenylethyl)-3-(trifluoromethyl)-1H-pyrazol-4-amine (intermediate 65C) and 406 mg (1.73 mmol) 2-carbamoyl-7-fluoroquinoline-4-carboxylic acid (intermediate 37A) were reacted to give after purification via a Biotage chromatography system (25 g snap KP-Sil column, hexane/0-100% ethyl acetate, then ethyl acetate/0-100% methanol) 816 mg (97%) and a subsequent purification of 200 mg of the 816 mg via HPLC (method 5d) 86 mg (9.9%) of the desired title compound.

¹H-NMR (400 MHz, DMSO d₆) δ (ppm)=1.85 (d, 3H), 2.22 (s, 3H), 5.84 (q, 1H), 7.24-7.43 (m, 5H), 7.76-7.85 (m, 1H), 7.93 (dd, 1H), 7.97 (br. S., 1H), 8.24-8.33 (m, 2H), 8.40 (br. S., 1H), 10.42 (s, 1H).

### Example 335 and Example 336

### (R or S)-7-fluoro-N⁴-[5-methyl-1-(1-phenylethyl)-3-(trifluoromethyl)-1H-pyrazol-4-yl]quinoline-2,4-dicarboxamide and (S or R)-7-fluoro-N⁴-[5-methyl-1-(1-phenylethyl)-3-(trifluoromethyl)-1H-pyrazol-4-yl]quinoline-2,4-dicarboxamide

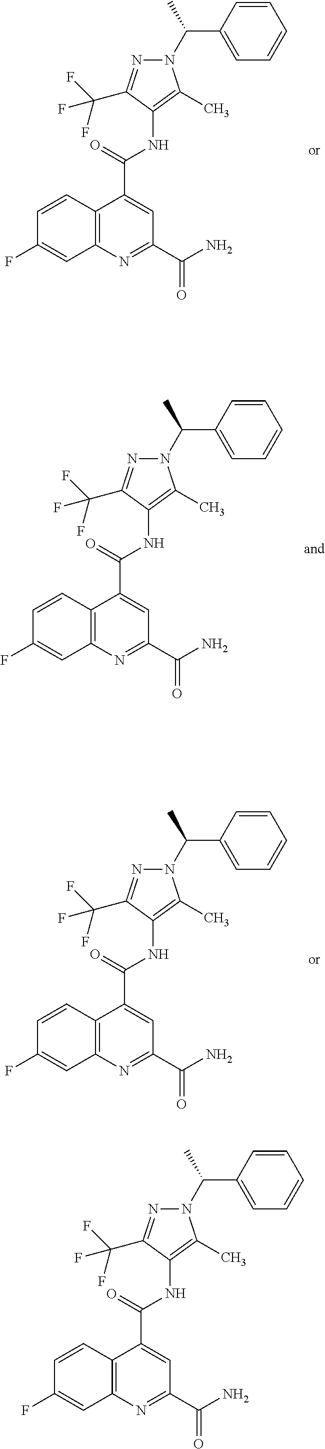 [SMILES: Cc1c(NC(=O)c2cc(C(N)=O)nc3cc(F)ccc23)c(C(F)(F)F)nn1[C@H](C)c1ccccc1 ; Cc1c(NC(=O)c2cc(C(N)=O)nc3cc(F)ccc23)c(C(F)(F)F)nn1[C@@H](C)c1ccccc1 ; Cc1c(NC(=O)c2cc(C(N)=O)nc3cc(F)ccc23)c(C(F)(F)F)nn1[C@@H](C)c1ccccc1 ; Cc1c(NC(=O)c2cc(C(N)=O)nc3cc(F)ccc23)c(C(F)(F)F)nn1[C@H](C)c1ccccc1]

816 mg of the racemic mixture of the title compound from example 334 was separated via a chiral HPLC (method 8) to give 128 mg of example 335 (Rt 7.5-10 min.) and 45 mg (Rt 11.5-15.5 min.) of example 336 together with 279 mg of a mixture of both enantiomers.

### Example 337

### 6-cyano N⁴-[1-(4-cyanobenzyl)-5-methyl-3-(trifluoromethyl)-1H-pyrazol-4-yl]quinoline-2,4-dicarboxamide

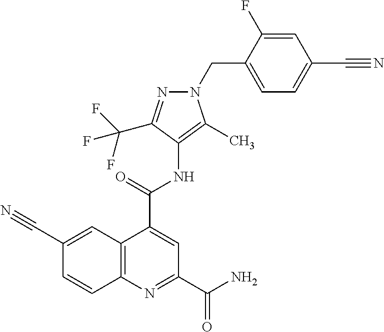 [SMILES: Cc1c(NC(=O)c2cc(C(N)=O)nc3ccc(C#N)cc23)c(C(F)(F)F)nn1Cc1ccc(C#N)cc1F]

A solution of 150 mg (0.27 mmol) N⁴-[1-(4-cyanobenzyl)-5-methyl-3-(trifluoromethyl)-1H-pyrazol-4-yl]-5-fluoroquinoline-2,4-dicarboxamide (example 250), 34.8 mg (0.30 mmol) zinc cyanide and 31.1 mg (0.027 mmol) Pd(PPh₃)₄ in 6.1 mL DMF was heated up to 150° C. for 2 hours. Then the same amount of zinc cyanide and Pd(PPh₃)₄ was added and the mixture was heated for 2 hours at 150° C., 60° C. for 14 hours and 7 hours at 150° C. After cooling to room temperature the reaction mixture was diluted with ethyl acetate. The organic phase was washed with sodium hydrogencarbonate and brine, dried over sodium sulfate, filtered and evaporated to dryness. The crude product was purified via preparative HPLC (method 5d) to give 50 mg (35%) of the desired title compound.

¹H-NMR (400 MHz, DMSO d₆) δ (ppm)=2.28 (s, 3H), 5.60 (s, 2H), 7.39 (d, 2H), 7.89 (d, 2H), 8.04 (br. s., 1H), 8.24 (dd, 1H), 8.35 (d, 1H), 8.44 (s, 1H), 8.49 (s, 1H), 8.69-8.72 (m, 1H), 10.57 (br. s., 1H).

### Example 338

### 8-chloro-N⁴-[1-(4-cyanobenzyl)-5-methyl-3-(trifluoromethyl)-1H-pyrazol-4-yl]quinoline-2,4-dicarboxamide

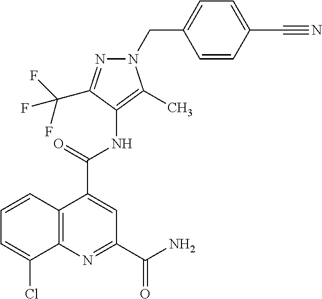 [SMILES: Cc1c(NC(=O)c2cc(C(N)=O)nc3c(Cl)cccc23)c(C(F)(F)F)nn1Cc1ccc(C#N)cc1]

In analogy to example 118), 134 mg (0.48 mmol) 4-{[4-amino-5-methyl-3-(trifluoromethyl)-1H-pyrazol-1-yl]methyl}benzonitrile (intermediate 26C) and 100 mg (0.40 mmol) 2-carbamoyl-8-chloroquinoline-4-carboxylic acid (intermediate 43A) were reacted to give after purification via preparative HPLC (method 4) 29 mg (14%) of the desired title compound.

¹H-NMR (400 MHz, DMSO d₆) δ (ppm)=2.27 (s, 3H), 5.60 (s, 2H), 7.38 (d, 2H), 7.77-7.82 (m, 1H), 7.87-7.91 (m, 2H), 8.05-8.18 (m, 4H), 8.34 (s, 1H), 10.48 (s, 1H).

### Example 339

### N⁴-[1-(4-cyanobenzyl)-5-methyl-3-(trifluoromethyl)-1H-pyrazol-4-yl]-7-methoxyquinoline-2,4-dicarboxamide

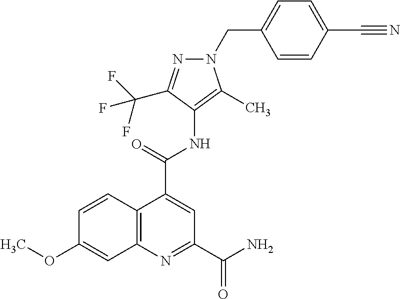 [SMILES: COc1ccc2c(C(=O)Nc3c(C(F)(F)F)nn(Cc4ccc(C#N)cc4)c3C)cc(C(N)=O)nc2c1]

In analogy to example 118), 205 mg (0.73 mmol) 4-{[4-amino-5-methyl-3-(trifluoromethyl)-1H-pyrazol-1-yl]methyl}benzonitrile (intermediate 26C) and 150 mg (0.61 mmol) 2-carbamoyl-7-methoxyquinoline-4-carboxylic acid (intermediate 42A) were reacted to give after purification via a Biotage chromatography system (10 g snap KP-Sil column, hexane/40-100% ethyl acetate), then via two subsequent preparative HPLC (method 5d) 36 mg (10%) of the desired title compound.

¹H-NMR (400 MHz, DMSO d₆) δ (ppm)=2.25 (s, 3H), 3.97 (s, 3H), 5.60 (s, 2H), 7.38 (d, 2H), 7.47 (dd, 1H), 7.55 (d, 1H), 7.84-7.91 (m, 3H), 8.08-8.12 (m, 2H), 8.30-8.34 (m, 1H), 10.37 (s, 1H).

### Example 340

### 2-(azetidin-1-ylcarbonyl)-N-[1-(4-cyanobenzyl)-5-methyl-3-(trifluoromethyl)-1H-pyrazol-4-yl]-7-fluoroquinoline-4-carboxamide

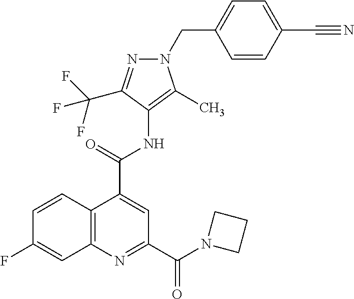 [SMILES: Cc1c(NC(=O)c2cc(C(=O)N3CCC3)nc3cc(F)ccc23)c(C(F)(F)F)nn1Cc1ccc(C#N)cc1]

In analogy to example 118), 97 mg (0.35 mmol) 4-{[4-amino-5-methyl-3-(trifluoromethyl)-1H-pyrazol-1-yl]methyl}benzonitrile (intermediate 26C) and 105 mg (0.29 mmol) 2-(azetidin-1-ylcarbonyl)-7-fluoroquinoline-4-carboxylic acid (intermediate 52A) were reacted to give a solid which was filtered and washed with THF. After drying of the solid we obtained 38 mg (24%) of the desired title compound.

¹H-NMR (400 MHz, DMSO d₆) δ (ppm)=2.25 (s, 3H), 2.34 (quin, 2H), 4.16 (t, 2H), 4.74 (t, 2H), 5.60 (s, 2H), 7.38 (d, 2H), 7.77 (td, 1H), 7.89 (d, 2H), 7.95 (dd, 1H), 8.12 (s, 1H), 8.25 (dd, 1H), 10.44 (s, 1H).

### Example 341

### N⁴-[1-(4-cyanobenzyl)-5-methyl-3-(trifluoromethyl)-1H-pyrazol-4-yl]-7-fluoro-N²-(3-hydroxypropyl)quinoline-2,4-dicarboxamide

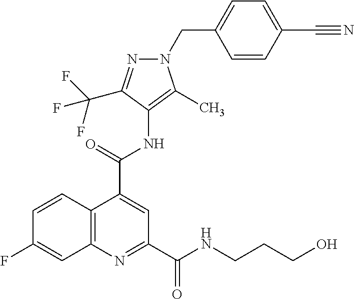 [SMILES: Cc1c(NC(=O)c2cc(C(=O)NCCCO)nc3cc(F)ccc23)c(C(F)(F)F)nn1Cc1ccc(C#N)cc1]

In analogy to example 118), 139 mg (0.50 mmol) 4-{[4-amino-5-methyl-3-(trifluoromethyl)-1H-pyrazol-1-yl]methyl}benzonitrile (intermediate 26C) and 192 mg (0.50 mmol) of the raw material in which 7-fluoro-2-[(3-hydroxypropyl)carbamoyl]quinoline-4-carboxylic acid (intermediate 53A) is included, were reacted to give after two subsequent purification via preparative HPLC (method 4) 10 mg (4.2%) of the desired title compound.

¹H-NMR (400 MHz, DMSO d₆) δ (ppm)=1.76 (quin, 2H), 2.28 (s, 3H), 3.42-3.56 (m, 4H), 4.57 (t, 1H), 5.62 (s, 2H), 7.40 (d, 2H), 7.80 (td, 1H), 7.86-7.96 (m, 3H), 8.26-8.36 (m, 2H), 9.08 (t, 1H), 10.49 (s, 1H).

### Example 342

### N⁴-[1-(4-cyanobenzyl)-5-methyl-3-(trifluoromethyl)-1H-pyrazol-4-yl]-7-fluoro-N²-[2-(morpholin-4-yl)ethyl]quinoline-2,4-dicarboxamide

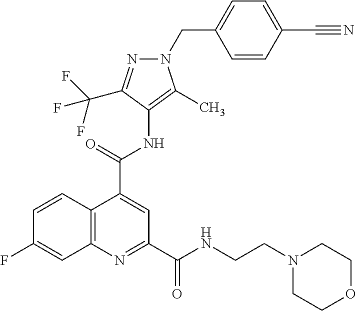 [SMILES: Cc1c(NC(=O)c2cc(C(=O)NCCN3CCOCC3)nc3cc(F)ccc23)c(C(F)(F)F)nn1Cc1ccc(C#N)cc1]

In analogy to example 118), 121 mg (0.43 mmol) 4-{[4-amino-5-methyl-3-(trifluoromethyl)-1H-pyrazol-1-yl]methyl}benzonitrile (intermediate 26C) and 121 mg (0.43 mmol) of the raw material in which 7-fluoro-2-{[2-(morpholin-4-yl)ethyl]carbamoyl}quinoline-4-carboxylic acid (intermediate 54A) is included, were reacted to give after a purification via preparative HPLC (method 4) 43 mg (18%) of the desired title compound.

¹H-NMR (400 MHz, DMSO d) δ (ppm)=2.28 (s, 3H), 2.42-2.48 (m, 4H), 2.55 (t, 2H), 3.52 (q, 2H), 3.58-3.63 (m, 4H), 5.62 (s, 2H), 7.40 (d, 2H), 7.78-7.86 (m, 1H), 7.87-7.99 (m, 3H), 8.26-8.34 (m, 2H), 9.00 (t, 1H), 10.50 (s, 1H).

### Example 343

### N⁴-[1-(4-cyanobenzyl)-5-methyl-3-(trifluoromethyl)-1H-pyrazol-4-yl]-7-fluoro-6-methylquinoline-2,4-dicarboxamide

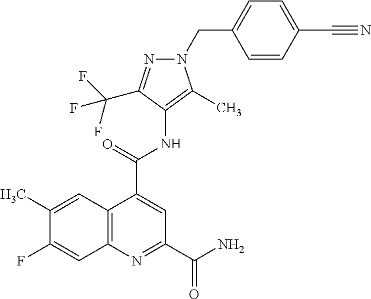 [SMILES: Cc1cc2c(C(=O)Nc3c(C(F)(F)F)nn(Cc4ccc(C#N)cc4)c3C)cc(C(N)=O)nc2cc1F]

In analogy to example 118), 135 mg (0.48 mmol) 4-{[4-amino-5-methyl-3-(trifluoromethyl)-1H-pyrazol-1-yl]methyl}benzonitrile (intermediate 26C) and 100 mg (0.40 mmol) 2-carbamoyl-7-fluoro-6-methylquinoline-4-carboxylic acid (intermediate 50A) were reacted to give after a purification via preparative HPLC (method 4) 83 mg (39%) of the desired title compound.

¹H-NMR (500 MHz, DMSO d₆) δ (ppm)=2.26 (s, 3H), 2.47 (s, 3H), 5.60 (s, 2H), 7.39 (d, 2H), 7.86-7.93 (m, 4H), 8.10 (d, 1H), 8.23 (s, 1H), 8.34 (d, 1H), 10.44 (s, 1H).

### Example 344

### N⁴-[1-(4-cyanobenzyl)-5-methyl-3-(trifluoromethyl)-1H-pyrazol-4-yl]-6-[(2-methoxyethyl)amino]quinoline-2,4-dicarboxamide

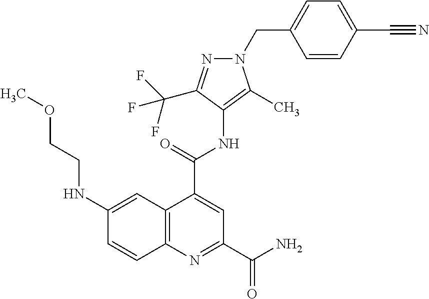 [SMILES: COCCNc1ccc2nc(C(N)=O)cc(C(=O)Nc3c(C(F)(F)F)nn(Cc4ccc(C#N)cc4)c3C)c2c1]

In analogy to example 118), 82 mg (0.29 mmol) 4-{[4-amino-5-methyl-3-(trifluoromethyl)-1H-pyrazol-1-yl]methyl}benzonitrile (intermediate 26C) and 71 mg (0.24 mmol) 2-carbamoyl-6-[(2-methoxyethyl)amino]quinoline-4-carboxylic acid (intermediate 55A) were reacted to give after a purification via preparative HPLC (method 4) 33 mg (23%) of the desired title compound.

¹H-NMR (500 MHz, DMSO d₆) δ (ppm)=2.25 (s, 3H), 3.23-3.28 (m, 5H), 3.53 (t, 2H), 5.59 (s, 2H), 6.76 (t, 1H), 6.92 (d, 1H), 7.37-7.41 (m, 3H), 7.61 (d, 1H), 7.85 (d, 1H), 7.88 (d, 2H), 8.03 (s, 1H), 8.09 (d, 1H), 10.19 (s, 1H).

### Example 345

### N⁴-[1-(4-cyanobenzyl)-5-methyl-3-(trifluoromethyl)-1H-pyrazol-4-yl]-6-(piperidin-1-yl)quinoline-2,4-dicarboxamide

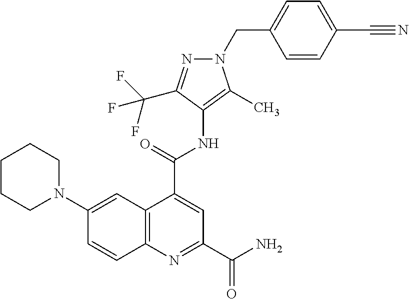 [SMILES: Cc1c(NC(=O)c2cc(C(N)=O)nc3ccc(N4CCCCC4)cc23)c(C(F)(F)F)nn1Cc1ccc(C#N)cc1]

In analogy to example 118), 27 mg (0.096 mmol) 4-{[4-amino-5-methyl-3-(trifluoromethyl)-1H-pyrazol-1-yl]methyl}benzonitrile (intermediate 26C) and 24 mg (0.080 mmol) 2-carbamoyl-6-(piperidin-1-yl)quinoline-4-carboxylic acid (intermediate 56A) were reacted to give after a purification via preparative HPLC (method 5d) 11 mg (23%) of the desired title compound.

¹H-NMR (500 MHz, DMSO d₆) δ (ppm)=1.59-1.69 (m, 6H), 2.27 (s, 3H), 3.35-3.43 (m, 4H), 5.61 (s, 2H), 7.32 (d, 1H), 7.41 (d, 2H), 7.72 (d, 1H), 7.79 (dd, 1H), 7.90 (d, 2H), 7.98 (d, 1H), 8.10 (s, 1H), 8.16-8.24 (m, 1H), 10.29 (s, 1H).

### Example 346

### 6-chloro-N⁴-{1-[3-(4-cyanophenyl)propyl]-3,5-dimethyl-1H-pyrazol-4-yl}-7-fluoroquinoline-2,4-dicarboxamide

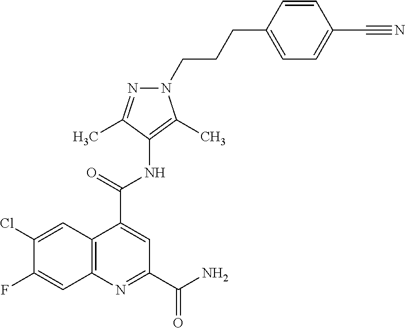 [SMILES: Cc1nn(CCCc2ccc(C#N)cc2)c(C)c1NC(=O)c1cc(C(N)=O)nc2cc(F)c(Cl)cc12]

In analogy to example 118), 114 mg (0.45 mmol) 4-[3-(4-amino-3,5-dimethyl-1H-pyrazol-1-yl)propyl]benzonitrile (intermediate 66C) and 100 mg (0.37 mmol) 2-carbamoyl-6-chloro-7-fluoroquinoline-4-carboxylic acid (intermediate 32A) were reacted to give after a purification via preparative HPLC (method 4) 26 mg (13%) of the desired title compound.

¹H-NMR (400 MHz, DMSO d₆) δ (ppm)=2.07 (quin, 2H), 2.12 (s, 3H), 2.18 (s, 3H), 2.72 (t, 2H), 4.00 (t, 2H), 7.47 (d, 2H), 7.75-7.80 (m, 2H), 8.01 (s, 1H), 8.14 (d, 1H), 8.38 (s, 1H), 8.41 (d, 1H), 8.51 (d, 1H), 10.18 (s, 1H).

### Example 347

### N⁴-{1-[3-(4-cyanophenyl)propyl]-3,5-dimethyl-1H-pyrazol-4-yl}quinoline-2,4-dicarboxamide

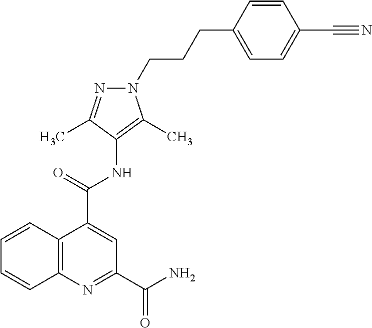 [SMILES: Cc1nn(CCCc2ccc(C#N)cc2)c(C)c1NC(=O)c1cc(C(N)=O)nc2ccccc12]

In analogy to example 118), 141 mg (0.56 mmol) 4-[3-(4-amino-3,5-dimethyl-1H-pyrazol-1-yl)propyl]benzonitrile (intermediate 66C) and 100 mg (0.46 mmol) 2-carbamoylquinoline-4-carboxylic acid (intermediate 4A) were reacted to give after a purification via preparative HPLC (method 4) 74 mg (34%) of the desired title compound.

¹H-NMR (400 MHz, DMSO d₆) δ (ppm)=2.07 (quin, 2H), 2.13 (s, 3H), 2.20 (s, 3H), 2.73 (t, 2H), 4.00 (t, 2H), 7.47 (d, 2H), 7.75-7.80 (m, 2H), 7.80-7.85 (m, 1H), 7.91 (d, 1H), 7.93-7.98 (m, 1H), 8.22 (d, 1H), 8.26 (dd, 1H), 8.28 (s, 1H), 8.41 (d, 1H), 10.04 (s, 1H).

### Example 348

### 6-bromo-N-{1-[3-(4-cyanophenyl)propyl]-3,5-dimethyl-1H-pyrazol-4-yl}-2-(trifluoromethyl)quinoline-4-carboxamide

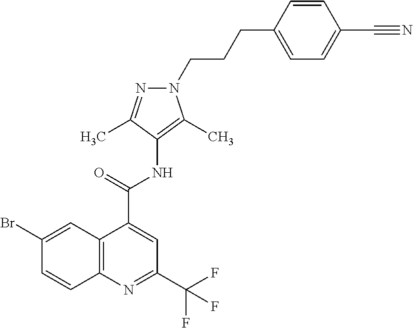 [SMILES: Cc1nn(CCCc2ccc(C#N)cc2)c(C)c1NC(=O)c1cc(C(F)(F)F)nc2ccc(Br)cc12]

In analogy to example 118), 95 mg (0.38 mmol) 4-[3-(4-amino-3,5-dimethyl-1H-pyrazol-1-yl)propyl]benzonitrile (intermediate 66C) and 100 mg (0.31 mmol) 6-bromo-2-(trifluoromethyl)quinoline-4-carboxylic acid (intermediate 1A) were reacted to give after a purification via preparative HPLC (method 4) 66 mg (37%) of the desired title compound.

¹H-NMR (400 MHz, DMSO d₆) δ (ppm)=2.07 (quin, 2H), 2.14 (s, 3H), 2.20 (s, 3H), 2.72 (t, 2H), 4.00 (t, 2H), 7.47 (d, 2H), 7.75-7.81 (m, 2H), 8.16 (dd, 1H), 8.24 (d, 1H), 8.29 (s, 1H), 8.50 (d, 1H), 10.14 (s, 1H).

### Example 349

### N-{1-[3-(4-cyanophenyl)propyl]-3,5-dimethyl-1H-pyrazol-4-yl}-2-methoxyquinoline-4-carboxamide

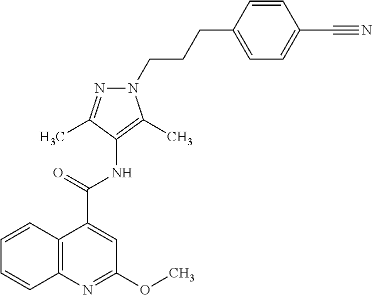 [SMILES: COc1cc(C(=O)Nc2c(C)nn(CCCc3ccc(C#N)cc3)c2C)c2ccccc2n1]

In analogy to example 118), 150 mg (0.59 mmol) 4-[3-(4-amino-3,5-dimethyl-1H-pyrazol-1-yl)propyl]benzonitrile (intermediate 66C) and 100 mg (0.49 mmol) 2-methoxyquinoline-4-carboxylic acid were reacted to give after a purification via preparative HPLC (method 4) 43 mg (19%) of the desired title compound.

¹H-NMR (400 MHz, DMSO d₆) δ (ppm)=2.06 (quin, 2H), 2.11 (s, 3H), 2.18 (s, 3H), 2.72 (t, 2H), 3.98 (t, 2H), 4.05 (s, 3H), 7.22 (s, 1H), 7.47 (d, 2H), 7.50-7.56 (m, 1H), 7.68-7.81 (m, 3H), 7.87 (d, 1H), 8.06 (d, 1H), 9.86 (s, 1H).

### Example 350

### 6-bromo-N⁴-{1-[(5-cyanopyridin-2-yl)methyl]-3,5-dimethyl-1H-pyrazol-4-yl}quinoline-2,4-dicarboxamide

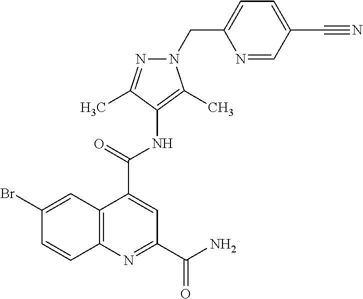 [SMILES: Cc1nn(Cc2ccc(C#N)cn2)c(C)c1NC(=O)c1cc(C(N)=O)nc2ccc(Br)cc12]

In analogy to example 118), 69 mg (0.31 mmol) 6-[(4-amino-3,5-dimethyl-1H-pyrazol-1-yl)methyl]nicotinonitrile (intermediate 67C) and 75 mg (0.25 mmol) 6-bromo-2-carbamoylquinoline-4-carboxylic acid (intermediate 2A) were reacted to give after a purification via preparative HPLC (method 4) 18 mg (13%) of the desired title compound.

¹H-NMR (500 MHz, DMSO d₆) δ (ppm)=2.12 (s, 3H), 2.21 (s, 3H), 5.46 (s, 2H), 7.23 (d, 1H), 7.94 (s, 1H), 8.08 (dd, 1H), 8.14 (d, 1H), 8.32 (dd, 1H), 8.37 (s, 1H), 8.41 (d, 1H), 8.48 (d, 1H), 9.00 (dd, 1H), 10.20 (s, 1H).

### Example 351

### 6-chloro-N⁴-{1-[(5-cyanopyridin-2-yl)methyl]-3,5-dimethyl-1H-pyrazol-4-yl}-7-fluoroquinoline-2,4-dicarboxamide

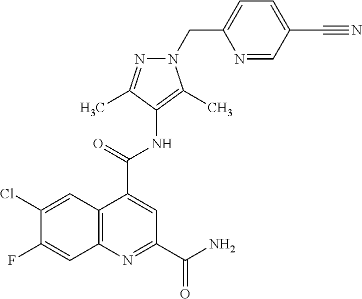 [SMILES: Cc1nn(Cc2ccc(C#N)cn2)c(C)c1NC(=O)c1cc(C(N)=O)nc2cc(F)c(Cl)cc12]

In analogy to example 118), 108 mg (0.48 mmol) 6-[(4-amino-3,5-dimethyl-1H-pyrazol-1-yl)methyl]nicotinonitrile (intermediate 67C) and 106 mg (0.40 mmol) 2-carbamoyl-6-chloro-7-fluoroquinoline-4-carboxylic acid (intermediate 32A) were reacted to give after a purification via preparative HPLC (method 3) 15 mg (7.7%) of the desired title compound.

¹H-NMR (400 MHz, DMSO d₆) δ (ppm)=2.13 (s, 3H), 2.22 (s, 3H), 5.47 (s, 2H), 7.24 (d, 1H), 8.01 (s, 1H), 8.14 (d, 1H), 8.34 (dd, 1H), 8.38-8.46 (m, 2H), 8.53 (d, 1H), 9.01 (dd, 1H), 10.26 (s, 1H).

### Example 352

### N⁴-{1-[(5-cyanopyridin-2-yl)methyl]-3,5-dimethyl-1H-pyrazol-4-yl}-5-fluoroquinoline-2,4-dicarboxamide

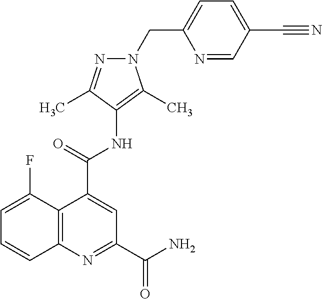 [SMILES: Cc1nn(Cc2ccc(C#N)cn2)c(C)c1NC(=O)c1cc(C(N)=O)nc2cccc(F)c12]

In analogy to example 118), 87 mg (0.38 mmol) 6-[(4-amino-3,5-dimethyl-1H-pyrazol-1-yl)methyl]nicotinonitrile (intermediate 67C) and 75 mg (0.32 mmol) 2-carbamoyl-5-fluoroquinoline-4-carboxylic acid (intermediate 39A) were reacted to give after a purification via preparative HPLC (method 4) 58 mg (36%) of the desired title compound.

¹H-NMR (400 MHz, DMSO d₆) δ (ppm)=2.12 (s, 3H), 2.21 (s, 3H), 5.45 (s, 2H), 7.22 (d, 1H), 7.73-7.81 (m, 1H), 7.89-7.97 (m, 2H), 8.28 (s, 1H), 8.30-8.39 (m, 3H), 8.99 (dd, 1H), 10.14 (s, 1H).

### Example 353

### 6-bromo-N-{1-[(5-cyanopyridin-2-yl)methyl]-3,5-dimethyl-1H-pyrazol-4-yl}-2-(trifluoromethyl)quinoline-4-carboxamide

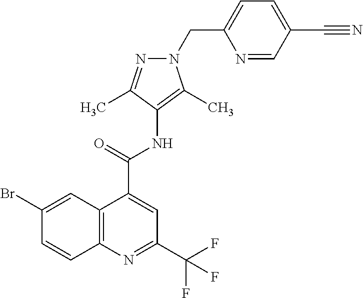 [SMILES: Cc1nn(Cc2ccc(C#N)cn2)c(C)c1NC(=O)c1cc(C(F)(F)F)nc2ccc(Br)cc12]

In analogy to example 118), 108 mg (0.48 mmol) 6-[(4-amino-3,5-dimethyl-1H-pyrazol-1-yl)methyl]nicotinonitrile (intermediate 67C) and 127 mg (0.40 mmol) 6-bromo-2-(trifluoromethyl)quinoline-4-carboxylic acid (intermediate 1A) were reacted to give after a purification via preparative HPLC (method 3) 76 mg (34%) of the desired title compound.

¹H-NMR (400 MHz, DMSO d₆) δ (ppm)=2.15 (s, 3H), 2.24 (s, 3H), 5.48 (s, 2H), 7.24 (d, 1H), 8.16 (dd, 1H), 8.24 (d, 1H), 8.30-8.39 (m, 2H), 8.52 (d, 1H), 9.01 (d, 1H), 10.23 (s, 1H).

### Example 354

### N⁴-{1-[(5-cyanopyridin-2-yl)methyl]-3,5-dimethyl-1H-pyrazol-4-yl}-6,7-difluoroquinoline-2,4-dicarboxamide

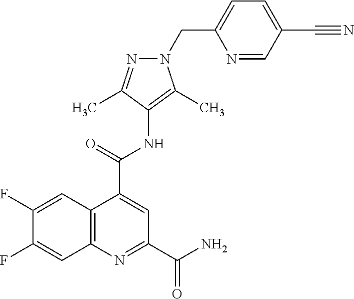 [SMILES: Cc1nn(Cc2ccc(C#N)cn2)c(C)c1NC(=O)c1cc(C(N)=O)nc2cc(F)c(F)cc12]

In analogy to example 118), 108 mg (0.48 mmol) 6-[(4-amino-3,5-dimethyl-1H-pyrazol-1-yl)methyl]nicotinonitrile (intermediate 67C) and 100 mg (0.40 mmol) 2-carbamoyl-6,7-difluoroquinoline-4-carboxylic acid (intermediate 3A) were reacted to give after a purification via preparative HPLC (method 3) 58 mg (30%) of the desired title compound.

¹H-NMR (400 MHz, DMSO d₆) δ (ppm)=2.13 (s, 3H), 2.22 (s, 3H), 5.47 (s, 2H), 7.24 (d, 1H), 7.99 (s, 1H), 8.18 (dd, 1H), 8.26 (dd, 1H), 8.34 (dd, 1H), 8.39 (br. d, 1H), 8.41 (s, 1H), 9.01 (dd, 1H), 10.24 (s, 1H).

### Example 355

### N⁴-{1-[(5-cyanopyridin-2-yl)methyl]-3,5-dimethyl-1H-pyrazol-4-yl}quinoline-2,4-dicarboxamide

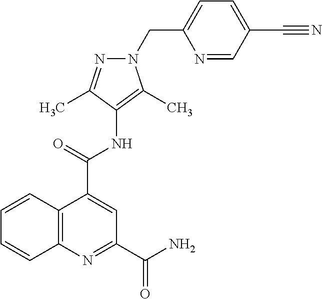 [SMILES: Cc1nn(Cc2ccc(C#N)cn2)c(C)c1NC(=O)c1cc(C(N)=O)nc2ccccc12]

In analogy to example 118), 108 mg (0.48 mmol) 6-[(4-amino-3,5-dimethyl-1H-pyrazol-1-yl)methyl]nicotinonitrile (intermediate 67C) and 86 mg (0.40 mmol) 2-carbamoylquinoline-4-carboxylic acid (intermediate 4A) were reacted to give after a purification via preparative HPLC (method 3) 46 mg (26%) of the desired title compound.

¹H-NMR (400 MHz, DMSO d₆) δ (ppm)=2.15 (s, 3H), 2.24 (s, 3H), 5.47 (s, 2H), 7.24 (d, 1H), 7.83 (ddd, 1H), 7.88-8.01 (m, 2H), 8.22 (d, 1H), 8.27 (d, 1H), 8.30 (s, 1H), 8.34 (dd, 1H), 8.41 (br. d, 1H), 9.02 (dd, 1H), 10.12 (s, 1H).

### Example 356

### N⁴-{1-[(5-cyanopyridin-2-yl)methyl]-3,5-dimethyl-1H-pyrazol-4-yl}-7-fluoroquinoline-2,4-dicarboxamide

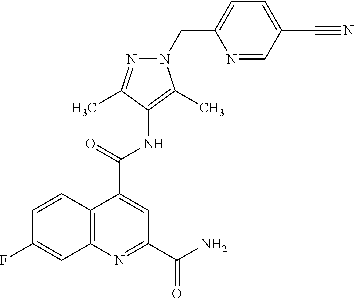 [SMILES: Cc1nn(Cc2ccc(C#N)cn2)c(C)c1NC(=O)c1cc(C(N)=O)nc2cc(F)ccc12]

In analogy to example 118), 87 mg (0.38 mmol) 6-[(4-amino-3,5-dimethyl-1H-pyrazol-1-yl)methyl]nicotinonitrile (intermediate 67C) and 75 mg (0.32 mmol) 2-carbamoyl-7-fluoroquinoline-4-carboxylic acid (intermediate 37A) were reacted to give after a purification via preparative HPLC (method 4) 51 mg (33%) of the desired title compound.

¹H-NMR (400 MHz, DMSO d₆) δ (ppm)=2.12 (s, 3H), 2.21 (s, 3H), 5.45 (s, 2H), 7.22 (d, 1H), 7.77 (ddd, 1H), 7.88-7.97 (m, 2H), 8.28 (s, 1H), 8.30-8.39 (m, 3H), 8.99 (dd, 1H), 10.14 (s, 1H).

### Example 357

### N-{1-[(5-cyanopyridin-2-yl)methyl]-3,5-dimethyl-1H-pyrazol-4-yl}-2-methoxyquinoline-4-carboxamide

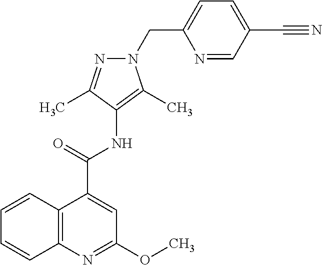 [SMILES: COc1cc(C(=O)Nc2c(C)nn(Cc3ccc(C#N)cn3)c2C)c2ccccc2n1]

In analogy to example 118), 108 mg (0.48 mmol) 6-[(4-amino-3,5-dimethyl-1H-pyrazol-1-yl)methyl]nicotinonitrile (intermediate 67C) and 80 mg (0.40 mmol) 2-methoxyquinoline-4-carboxylic acid were reacted to give after a purification via preparative HPLC (method 3) 98 mg (54%) of the desired title compound.

¹H-NMR (400 MHz, DMSO d₆) δ (ppm)=2.13 (s, 3H), 2.22 (s, 3H), 4.05 (s, 3H), 5.46 (s, 2H), 7.22 (d, 1H), 7.24 (s, 1H), 7.53 (ddd, 1H), 7.74 (ddd, 1H), 7.87 (d, 1H), 8.07 (dd, 1H), 8.33 (dd, 1H), 9.01 (dd, 1H), 9.95 (s, 1H).

### Example 358

### 6-bromo-N⁴-{1-[(5-cyanopyridin-2-yl)methyl]-5-methyl-3-(trifluoromethyl)-1H-pyrazol-4-yl}quinoline-2,4-dicarboxamide

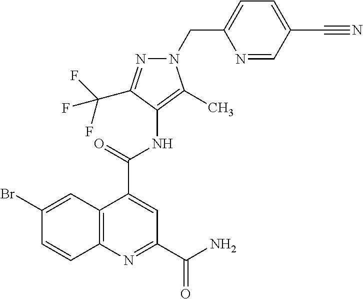 [SMILES: Cc1c(NC(=O)c2cc(C(N)=O)nc3ccc(Br)cc23)c(C(F)(F)F)nn1Cc1ccc(C#N)cn1]

In analogy to example 118), 86 mg (0.31 mmol) 6-{[4-amino-5-methyl-3-(trifluoromethyl)-1H-pyrazol-1-yl]methyl}nicotinonitrile (intermediate 68C) and 75 mg (0.25 mmol) 6-bromo-2-carbamoylquinoline-4-carboxylic acid (intermediate 2A) were reacted to give after a purification via preparative HPLC (method 4) 37 mg (33%) of the desired title compound.

¹H-NMR (500 MHz, DMSO d₆) δ (ppm)=2.30 (s, 3H), 5.71 (s, 2H), 7.48 (d, 1H), 7.95 (d, 1H), 8.07-8.11 (m, 1H), 8.15 (d, 1H), 8.35 (s, 1H), 8.38 (dd, 1H), 8.40-8.43 (m, 2H), 9.00-9.02 (m, 1H), 10.50 (s, 1H).

### Example 359

### 6-chloro-N⁴-{1-[(5-cyanopyridin-2-yl)methyl]-5-methyl-3-(trifluoromethyl)-1H-pyrazol-4-yl}-7-fluoroquinoline-2,4-dicarboxamide

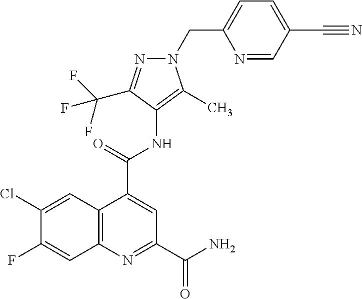 [SMILES: Cc1c(NC(=O)c2cc(C(N)=O)nc3cc(F)c(Cl)cc23)c(C(F)(F)F)nn1Cc1ccc(C#N)cn1]

In analogy to example 118), 175 mg (0.56 mmol) 6-{[4-amino-5-methyl-3-(trifluoromethyl)-1H-pyrazol-1-yl]methyl}nicotinonitrile (intermediate 68C) and 125 mg (0.47 mmol) 2-carbamoyl-6-chloro-7-fluoroquinoline-4-carboxylic acid (intermediate 32A) were reacted to give after a purification via preparative HPLC (method 3) 53 mg (20%) of the desired title compound.

¹H-NMR (400 MHz, DMSO d₆) δ (ppm)=2.31 (s, 3H), 5.73 (s, 2H), 7.50 (d, 1H), 8.03 (s, 1H), 8.16 (d, 1H), 8.35-8.51 (m, 5H), 9.01-9.07 (m, 1H), 10.57 (s, 1H).

### Example 360

### N⁴-{1-[(5-cyanopyridin-2-yl)methyl]-5-methyl-3-(trifluoromethyl)-1H-pyrazol-4-yl}-7-fluoroquinoline-2,4-dicarboxamide

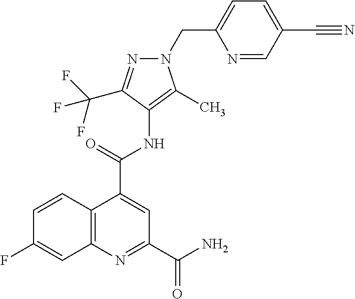 [SMILES: Cc1c(NC(=O)c2cc(C(N)=O)nc3cc(F)ccc23)c(C(F)(F)F)nn1Cc1ccc(C#N)cn1]

In analogy to example 118), 108 mg (0.38 mmol) 6-{[4-amino-5-methyl-3-(trifluoromethyl)-1H-pyrazol-1-yl]methyl}nicotinonitrile (intermediate 68C) and 75 mg (0.32 mmol) 2-carbamoyl-7-fluoroquinoline-4-carboxylic acid (intermediate 37A) were reacted to give after a purification via preparative HPLC (method 4) 52 mg (32%) of the desired title compound.

¹H-NMR (400 MHz, DMSO d₆) δ (ppm)=2.32 (s, 3H), 5.73 (s, 2H), 7.50 (d, 1H), 7.79-7.85 (m, 1H), 7.94 (dd, 1H), 7.98 (d, 1H), 8.27-8.34 (m, 2H), 8.37-8.45 (m, 2H), 9.02 (dd, 1H), 10.49 (s, 1H).

### Example 361

### N⁴-{1-[(5-cyanopyridin-2-yl)methyl]-5-methyl-3-(trifluoromethyl)-1H-pyrazol-4-yl}quinoline-2,4-dicarboxamide

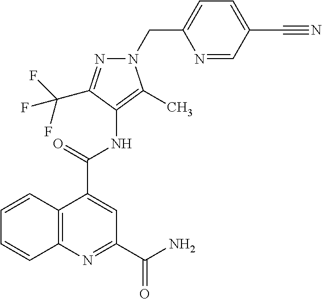 [SMILES: Cc1c(NC(=O)c2cc(C(N)=O)nc3ccccc23)c(C(F)(F)F)nn1Cc1ccc(C#N)cn1]

In analogy to example 118), 195 mg (0.62 mmol, purity about 90%) 6-{[4-amino-5-methyl-3-(trifluoromethyl)-1H-pyrazol-1-yl]methyl}nicotinonitrile (intermediate 68C) and 112 mg (0.52 mmol) 2-carbamoylquinoline-4-carboxylic acid (intermediate 4A) were reacted to give after a purification via preparative HPLC (method 3) 61 mg (24%) of the desired title compound.

¹H-NMR (400 MHz, DMSO d₆) δ (ppm)=2.31 (s, 3H), 5.71 (s, 2H), 7.48 (d, 1H), 7.82 (ddd, 1H), 7.90 (d, 1H), 7.94 (ddd, 1H), 8.19-8.23 (m, 2H), 8.27 (s, 1H), 8.34-8.41 (m, 2H), 9.01 (dd, 1H), 10.41 (s, 1H).

### Example 362

### 6-bromo-N-{1-[(5-cyanopyridin-2-yl)methyl]-5-methyl-3-(trifluoromethyl)-1H-pyrazol-4-yl}-2-(trifluoromethyl)quinoline-4-carboxamide

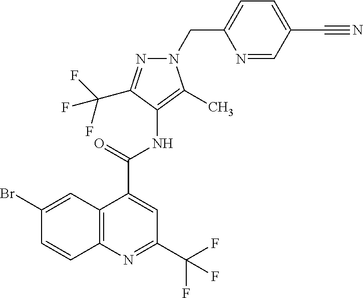 [SMILES: Cc1c(NC(=O)c2cc(C(F)(F)F)nc3ccc(Br)cc23)c(C(F)(F)F)nn1Cc1ccc(C#N)cn1]

In analogy to example 118), 150 mg (0.62 mmol, purity about 90%) 6-{[4-amino-5-methyl-3-(trifluoromethyl)-1H-pyrazol-1-yl]methyl}nicotinonitrile (intermediate 68C) and 128 mg (0.40 mmol) 6-bromo-2-(trifluoromethyl)quinoline-4-carboxylic acid (intermediate 1A) were reacted to give after a purification via preparative HPLC (method 3) 120 mg (50%) of the desired title compound.

¹H-NMR (400 MHz, DMSO d₆) δ (ppm)=2.34 (s, 3H), 5.73 (s, 2H), 7.50 (d, 1H), 8.17 (dd, 1H), 8.25 (d, 1H), 8.28 (s, 1H), 8.40 (dd, 1H), 8.44 (d, 1H), 9.02 (dd, 1H), 10.55 (s, 1H).

### Example 363

### N⁴-{1-[(5-cyanopyridin-2-yl)methyl]-5-methyl-3-(trifluoromethyl)-1H-pyrazol-4-yl}-5-fluoroquinoline-2,4-dicarboxamide

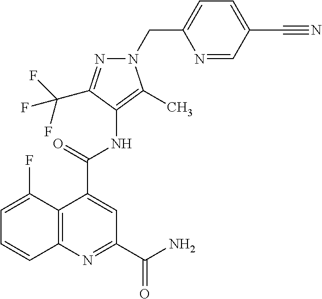 [SMILES: Cc1c(NC(=O)c2cc(C(N)=O)nc3cccc(F)c23)c(C(F)(F)F)nn1Cc1ccc(C#N)cn1]

In analogy to example 118), 108 mg (0.38 mmol) 6-{[4-amino-5-methyl-3-(trifluoromethyl)-1H-pyrazol-1-yl]methyl}nicotinonitrile (intermediate 68C) and 75 mg (0.32 mmol) 2-carbamoyl-5-fluoroquinoline-4-carboxylic acid (intermediate 39A) were reacted to give after a purification via preparative HPLC (method 4) 63 mg (38%) of the desired title compound.

¹H-NMR (400 MHz, DMSO d₆) δ (ppm)=2.30 (s, 3H), 5.71 (s, 2H), 7.48 (d, 1H), 7.80 (ddd, 1H), 7.92 (dd, 1H), 7.95 (br. s., 1H), 8.25-8.32 (m, 2H), 8.35-8.41 (m, 3H), 9.00 (dd, 1H), 10.46 (s, 1H).

### Example 364

### N⁴-{1-[(5-cyanopyridin-2-yl)methyl]-5-methyl-3-(trifluoromethyl)-1H-pyrazol-4-yl}-6,7-difluoroquinoline-2,4-dicarboxamide

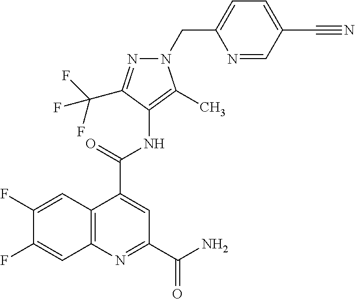 [SMILES: Cc1c(NC(=O)c2cc(C(N)=O)nc3cc(F)c(F)cc23)c(C(F)(F)F)nn1Cc1ccc(C#N)cn1]

In analogy to example 118), 188 mg (0.58 mmol, purity about 90%) 6-{[4-amino-5-methyl-3-(trifluoromethyl)-1H-pyrazol-1-yl]methyl}nicotinonitrile (intermediate 68C) and 121 mg (0.48 mmol) 2-carbamoyl-6,7-difluoroquinoline-4-carboxylic acid (intermediate 3A) were reacted to give after a purification via preparative HPLC (method 3) 65 mg (22%) of the desired title compound.

¹H-NMR (400 MHz, DMSO d₆) δ (ppm)=2.31 (s, 3H), 5.73 (s, 2H), 7.50 (d, 1H), 8.01 (br. s., 1H), 8.13-8.23 (m, 2H), 8.37-8.42 (m, 3H), 9.01-9.04 (m, 1H), 10.55 (s, 1H).

### Example 365

### N-{1-[(5-cyanopyridin-2-yl)methyl]-5-methyl-3-(trifluoromethyl)-1H-pyrazol-4-yl}-2-methoxyquinoline-4-carboxamide

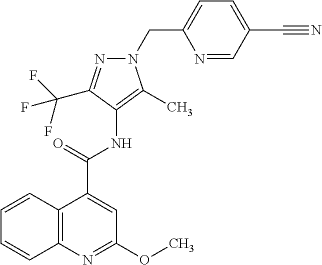 [SMILES: COc1cc(C(=O)Nc2c(C(F)(F)F)nn(Cc3ccc(C#N)cn3)c2C)c2ccccc2n1]

In analogy to example 118), 200 mg (0.64 mmol, purity about 90%) 6-{[4-amino-5-methyl-3-(trifluoromethyl)-1H-pyrazol-1-yl]methyl}nicotinonitrile (intermediate 68C) and 108 mg (0.53 mmol) 2-methoxyquinoline-4-carboxylic acid were reacted to give after a purification via preparative HPLC (method 3) 118 mg (46%) of the desired title compound.

¹H-NMR (400 MHz, DMSO d₆) δ (ppm)=2.30 (s, 3H), 4.05 (s, 3H), 5.72 (s, 2H), 7.19 (s, 1H), 7.49 (dd, 1H), 7.53 (ddd, 1H), 7.75 (ddd, 1H), 7.88 (d, 1H), 8.02 (dd, 1H), 8.39 (dd, 1H), 9.02 (dd, 1H), 10.28 (s, 1H).

### Example 366

### N⁴-[1-(4-cyanobenzyl)-5-methyl-3-(trifluoromethyl)-1H-pyrazol-4-yl]-6-methylquinoline-2,4-dicarboxamide

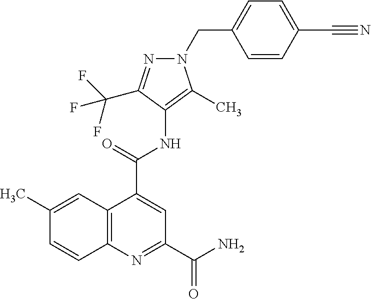 [SMILES: Cc1ccc2nc(C(N)=O)cc(C(=O)Nc3c(C(F)(F)F)nn(Cc4ccc(C#N)cc4)c3C)c2c1]

In analogy to example 118), 121 mg (0.43 mmol) 4-{[4-amino-5-methyl-3-(trifluoromethyl)-1H-pyrazol-1-yl]methyl}benzonitrile (intermediate 26C) and 100 mg (0.43 mmol) 2-carbamoyl-6-methylquinoline-4-carboxylic acid (intermediate 41A) were reacted to give after a purification via preparative HPLC (method 3) 18 mg (8.3%) of the desired title compound.

¹H-NMR (300 MHz, DMSO d₆) δ (ppm)=2.26 (s, 3H), 2.54 (s, 3H), 5.60 (s, 2H), 7.39 (d, 2H), 7.78 (dd, 1H), 7.85-7.92 (m, 3H), 7.95 (s, 1H), 8.10 (d, 1H), 8.22 (s, 1H), 8.35 (d, 1H), 10.39 (s, 1H).

### Example 367

### methyl 4-{[4-{[(2-carbamoyl-7-fluoroquinolin-4-yl)carbonyl]amino}-5-methyl-3-(trifluoromethyl)-1H-pyrazol-1-yl]methyl}benzoate

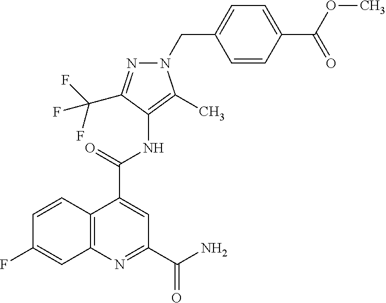 [SMILES: COC(=O)c1ccc(Cn2nc(C(F)(F)F)c(NC(=O)c3cc(C(N)=O)nc4cc(F)ccc34)c2C)cc1]

In analogy to example 118), 2.62 g (8.36 mmol) methyl 4-{[4-amino-5-methyl-3-(trifluoromethyl)-1H-pyrazol-1-yl]methyl}benzoate (intermediate 69C) and 1.63 g (6.97 mmol) 2-carbamoyl-7-fluoroquinoline-4-carboxylic acid (intermediate 37A) were reacted to give crude product, which was stirred in a mixture of ethzl acetate E methanol 9>1 for 1 hour at room temperature. After filtration we got a solid, which was dried to give 1.82 (47%). of the desired title compound.

¹H-NMR (300 MHz, DMSO d₆) δ (ppm)=2.26 (s, 3H), 3.84 (s, 3H), 5.58 (s, 2H), 7.36 (d, 2H), 7.79 (ddd, 1H), 7.92 (dd, 1H), 7.96-8.03 (m, 3H), 8.23-8.34 (m, 2H), 8.40 (br. s., 1H), 10.47 (s, 1H).

### Example 368

### 4-{[4-{[(2-carbamoyl-7-fluoroquinolin-4-yl)carbonyl]amino}-5-methyl-3-(trifluoromethyl)-1H-pyrazol-1-yl]methyl}benzoic acid

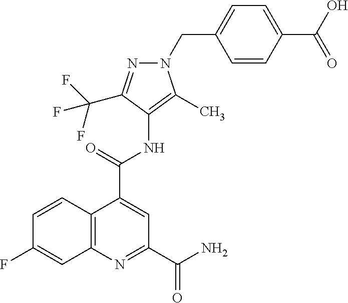 [SMILES: Cc1c(NC(=O)c2cc(C(N)=O)nc3cc(F)ccc23)c(C(F)(F)F)nn1Cc1ccc(C(=O)O)cc1]

To a solution of 1.00 g (1.89 mmol) of methyl 4-{[4-{[(2-carbamoyl-7-fluoroquinolin-4-yl)carbonyl]amino}-5-methyl-3-(trifluoromethyl)-1H-pyrazol-1-yl]methyl}benzoate (example 367) in 13.1 mL methanol and 10 mL THF was added a solution of 680 mg sodium hydroxide in 26.1 mL water. This mixture was stirred for 2 hours at 25° C. and then concentrated in vacuum. The residue was diluted with water and 10% aq. sulfuric acid was added up to pH 5. The formed solid was isolated by filtration and dried in vacuum to give 1.10 g (107%, containing moisture) of the desired title compound.

¹H-NMR (400 MHz, DMSO d₆) δ (ppm)=2.26 (s, 3H), 5.57 (s, 2H), 7.33 (d, 2H), 7.78 (ddd, 1H), 7.89-7.99 (m, 4H), 8.24-8.31 (m, 2H), 8.37 (d, 1H), 10.45 (s, 1H), 12.53 (br. s., 1H).

### Example 369

### N⁴-[1-(4-carbamoylbenzyl)-5-methyl-3-(trifluoromethyl)-1H-pyrazol-4-yl]-7-fluoroquinoline-2,4-dicarboxamide

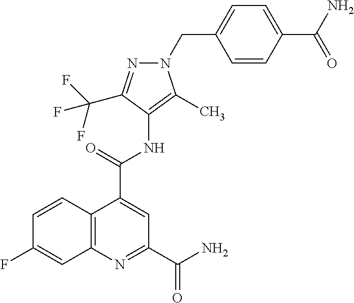 [SMILES: Cc1c(NC(=O)c2cc(C(N)=O)nc3cc(F)ccc23)c(C(F)(F)F)nn1Cc1ccc(C(N)=O)cc1]

In analogy to example 118), 0.51 mL (0.25 mmol) of a 0.5 M solution of ammonia in diaxane and 90 mg (0.17 mmol) 4-{[4-{[(2-carbamoyl-7-fluoroquinolin-4-yl)carbonyl]amino}-5-methyl-3-(trifluoromethyl)-1H-pyrazol-1-yl]methyl}benzoic acid (example 368) were reacted to give after a purification via preparative HPLC (method 5c) 33 mg (35%) of the desired title compound.

¹H-NMR (400 MHz, DMSO d₆) δ (ppm)=2.28 (s, 3H), 5.55 (s, 2H), 7.29 (d, 2H), 7.39 (br. s., 1H), 7.80 (ddd, 1H), 7.89 (d, 2H), 7.93 (dd, 1H), 7.98 (br. s., 2H), 8.27 (s, 1H), 8.30 (dd, 1H), 8.41 (s, 1H), 10.47 (s, 1H).

### Example 370

### 7-fluoro-N⁴-{5-methyl-1-[4-(methylcarbamoyl)benzyl]-3-(trifluoromethyl)-1H-pyrazol-4-yl}quinoline-2,4-dicarboxamide

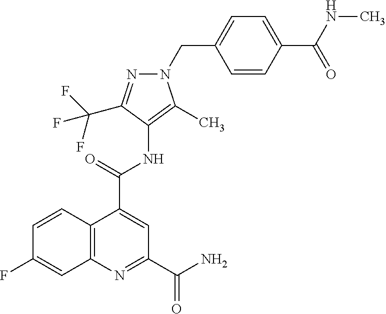 [SMILES: CNC(=O)c1ccc(Cn2nc(C(F)(F)F)c(NC(=O)c3cc(C(N)=O)nc4cc(F)ccc34)c2C)cc1]

In analogy to example 118), 0.21 mL (0.42 mmol) of a 2M solution of methylamine in THF and 180 mg (0.35 mmol) 4-{[4-{[(2-carbamoyl-7-fluoroquinolin-4-yl)carbonyl]amino}-5-methyl-3-(trifluoromethyl)-1H-pyrazol-1-yl]methyl}benzoic acid (example 368) were reacted to give after a purification via preparative HPLC (method 3) 14 mg (7.4%) of the desired title compound.

¹H-NMR (400 MHz, DMSO d₆) δ (ppm)=2.28 (s, 3H), 2.78 (d, 3H), 5.55 (s, 2H), 7.31 (d, 2H), 7.77-7.86 (m, 3H), 7.93 (dd, 1H), 7.98 (d, 1H), 8.27 (s, 1H), 8.30 (dd, 1H), 8.39-8.46 (m, 2H), 10.47 (s, 1H).

### Example 371

### N⁴-{1-[4-(dimethylcarbamoyl)benzyl]-5-methyl-3-(trifluoromethyl)-H-pyrazol-4-yl}-7-fluoroquinoline-2,4-dicarboxamide

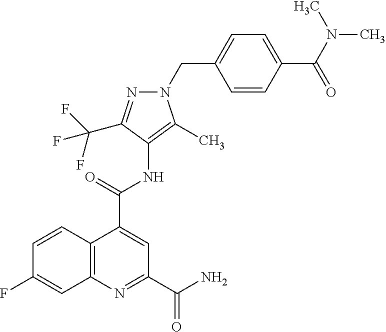 [SMILES: Cc1c(NC(=O)c2cc(C(N)=O)nc3cc(F)ccc23)c(C(F)(F)F)nn1Cc1ccc(C(=O)N(C)C)cc1]

In analogy to example 118), 0.21 mL (0.42 mmol) of a 2M solution of dimethylamine in THF and 180 mg (0.35 mmol) 4-{[4-{[(2-carbamoyl-7-fluoroquinolin-4-yl)carbonyl]amino}-5-methyl-3-(trifluoromethyl)-1H-pyrazol-1-yl]methyl}benzoic acid (example 368) were reacted to give after a purification via preparative HPLC (method 3) 115 mg (7.4%) of the desired title compound.

¹H-NMR (400 MHz, DMSO d₆) δ (ppm)=2.29 (s, 3H), 2.94 (d, 6H), 5.54 (s, 2H), 7.28 (d, 2H), 7.43-7.47 (m, 2H), 7.80 (ddd, 1H), 7.94 (dd, 1H), 7.98 (d, 1H), 8.27 (s, 1H), 8.30 (dd, 1H), 8.41 (d, 1H), 10.47 (s, 1H).

### Example 372

### N⁴-{1-[4-(azetidin-1-ylcarbonyl)benzyl]-5-methyl-3-(trifluoromethyl)-1H-pyrazol-4-yl}-7-fluoroquinoline-2,4-dicarboxamide

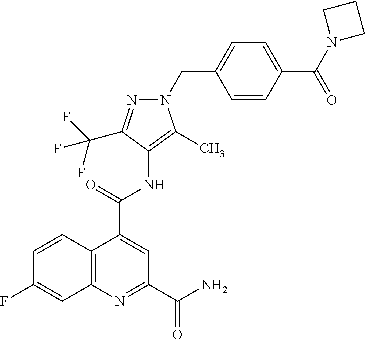 [SMILES: Cc1c(NC(=O)c2cc(C(N)=O)nc3cc(F)ccc23)c(C(F)(F)F)nn1Cc1ccc(C(=O)N2CCC2)cc1]

In analogy to example 118), 0.028 mL (0.42 mmol) of azetidine and 180 mg (0.35 mmol) 4-{[4-{[(2-carbamoyl-7-fluoroquinolin-4-yl)carbonyl]amino}-5-methyl-3-(trifluoromethyl)-1H-pyrazol-1-yl]methyl}benzoic acid (example 368) were reacted to give after a purification via preparative HPLC (method 4) 53 mg (26%) of the desired title compound.

¹H-NMR (400 MHz, DMSO d₆) δ (ppm)=0.95 (d, 2H), 2.21-2.31 (m, 5H), 4.04 (t, 2H), 4.30 (t, 2H), 5.55 (s, 2H), 7.30 (d, 2H), 7.64-7.68 (m, 2H), 7.81 (ddd, 1H), 7.94 (dd, 1H), 7.98 (d, 1H), 8.27 (s, 1H), 8.30 (dd, 1H), 8.41 (d, 1H), 10.48 (s, 1H).

### Example 373

### 7-fluoro-N⁴-[1-{4-[(2-methoxyethyl)carbamoyl]benzyl}-5-methyl-3-(trifluoromethyl)-1H-pyrazol-4-yl]quinoline-2,4-dicarboxamide

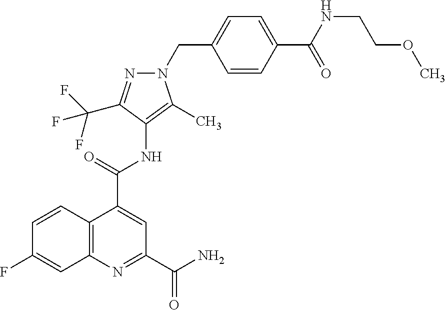 [SMILES: COCCNC(=O)c1ccc(Cn2nc(C(F)(F)F)c(NC(=O)c3cc(C(N)=O)nc4cc(F)ccc34)c2C)cc1]

In analogy to example 118), 31.5 mg (0.35 mmol) of 2-methoxyethanamine and 180 mg (0.35 mmol) 4-{[4-{[(2-carbamoyl-7-fluoroquinolin-4-yl)carbonyl]amino}-5-methyl-3-(trifluoromethyl)-1H-pyrazol-1-yl]methyl}benzoic acid (example 368) were reacted to give after a purification via preparative HPLC (method 3) 105 mg (51%) of the desired title compound.

¹H-NMR (400 MHz, DMSO d₆) δ (ppm)=2.28 (s, 3H), 3.26 (s, 3H), 3.39-3.49 (m, 4H), 5.55 (s, 2H), 7.31 (d, 2H), 7.80 (ddd, 1H), 7.87 (d, 2H), 7.93 (dd, 1H), 7.98 (d, 1H), 8.27 (s, 1H), 8.30 (dd, 1H), 8.41 (d, 1H), 8.52 (t, 1H), 10.47 (s, 1H).

### Example 374

### N⁴-[1-(4-{[2-(dimethylamino)ethyl]carbamoyl}benzyl)-5-methyl-3-(trifluoromethyl)-1H-pyrazol-4-yl]-7-fluoroquinoline-2,4-dicarboxamide

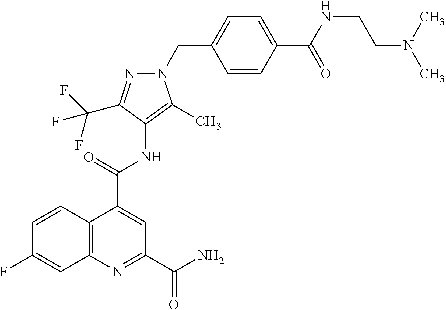 [SMILES: Cc1c(NC(=O)c2cc(C(N)=O)nc3cc(F)ccc23)c(C(F)(F)F)nn1Cc1ccc(C(=O)NCCN(C)C)cc1]

In analogy to example 118), 36.9 mg (0.42 mmol) of N,N-dimethylethane-1,2-diamine and 180 mg (0.35 mmol) 4-{[4-{[(2-carbamoyl-7-fluoroquinolin-4-yl)carbonyl]amino}-5-methyl-3-(trifluoromethyl)-1H-pyrazol-1-yl]methyl}benzoic acid (example 368) were reacted to give after a purification via preparative HPLC (method 4) 67 mg (32%) of the desired title compound.

¹H-NMR (400 MHz, DMSO d₆) δ (ppm)=2.17 (s, 6H), 2.28 (s, 3H), 2.39 (t, 2H), 3.35 (q, 2H), 5.55 (s, 2H), 7.31 (d, 2H), 7.77-7.87 (m, 1H), 7.93 (dd, 1H), 7.98 (d, 1H), 8.26-8.32 (m, 2H), 8.35-8.43 (m, 2H), 10.47 (s, 1H).

### Example 375

### 7-fluoro-N⁴-[1-{4-[(2-hydroxyethyl)carbamoyl]benzyl}-5-methyl-3-(trifluoromethyl)-1H-pyrazol-4-yl]quinoline-2,4-dicarboxamide

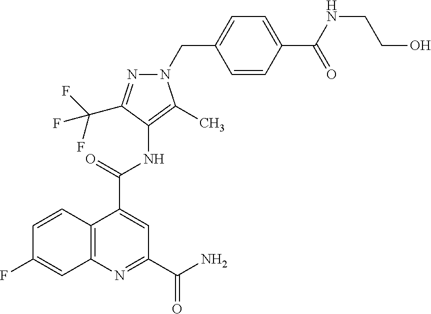 [SMILES: Cc1c(NC(=O)c2cc(C(N)=O)nc3cc(F)ccc23)c(C(F)(F)F)nn1Cc1ccc(C(=O)NCCO)cc1]

In analogy to example 118), 25.6 mg (0.42 mmol) of 2-aminoethanol and 180 mg (0.35 mmol) 4-{[4-{[(2-carbamoyl-7-fluoroquinolin-4-yl)carbonyl]amino}-5-methyl-3-(trifluoromethyl)-1H-pyrazol-1-yl]methyl}benzoic acid (example 368) were reacted to give after two subsequent purifications via preparative HPLC (method 3 and 5c) 22 mg (11%) of the desired title compound.

¹H-NMR (300 MHz, DMSO d₆) δ (ppm)=2.26 (s, 3H), 3.30 (q, 2H), 3.48 (t, 3H), 4.70 (br. s., 1H), 5.53 (s, 2H), 7.29 (d, 2H), 7.75-7.99 (m, 5H), 8.23-8.32 (m, 2H), 8.37-8.47 (m, 2H), 10.46 (s, 1H).

### Example 376

### (±)-ethyl [{4-[(4-{[(2-carbamoylquinolin-4-yl)carbonyl]amino}-3,5-dimethyl-1H-pyrazol-1-yl)methyl]phenyl}(methyl)oxido-λ⁶-sulfanylidene]carbamate

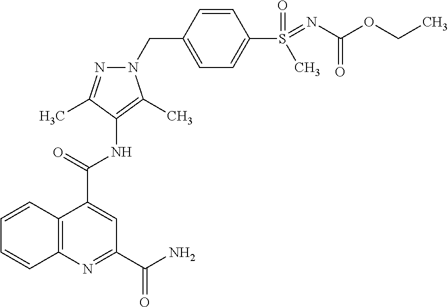 [SMILES: CCOC(=O)N=S(C)(=O)c1ccc(Cn2nc(C)c(NC(=O)c3cc(C(N)=O)nc4ccccc34)c2C)cc1]

In analogy to example 118), 500 mg (1.43 mmol) of (±)-ethyl [{4-[(4-amino-3,5-dimethyl-1H-pyrazol-1-yl)methyl]phenyl}(methyl)oxido-λ⁶-sulfanylidene]carbamate (intermediate 70C) and 257 mg (1.19 mmol) 2-carbamoylquinoline-4-carboxylic acid (intermediate 4A) were reacted to give after a purification via a Biotage chromatography system (25 g snap KP-Sil column, hexane/0-100% ethyl acetate, then ethyl acetate/0-10% methanol) 390 mg (60%) of the desired title compound. 40 mg thereof was further purified via preparative HPLC (method 5c) to yield 36 mg the desired title compound with a better purity.

¹H-NMR (400 MHz, DMSO d₆) δ (ppm)=1.09 (t, 3H), 2.16 (s, 3H), 2.23 (s, 3H), 3.45 (s, 3H), 3.85-3.98 (m, 2H), 5.41 (s, 2H), 7.46 (d, 2H), 7.80-7.85 (m, 1H), 7.89-7.99 (m, 4H), 8.22 (d, 1H), 8.27 (d, 1H), 8.29 (s, 1H), 8.40 (d, 1H), 10.11 (s, 1H).

### Example 377

### (±)-N⁴-{3,5-dimethyl-1-[4-(S-methylsulfonimidoyl)benzyl]-1H-pyrazol-4-yl}quinoline-2,4-dicarboxamide

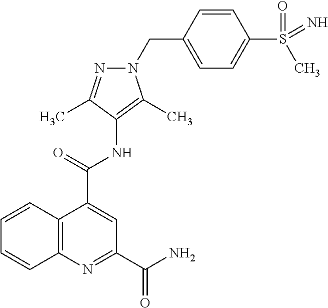 [SMILES: Cc1nn(Cc2ccc(S(C)(=N)=O)cc2)c(C)c1NC(=O)c1cc(C(N)=O)nc2ccccc12]

To a solution of 225 mg (0.41 mmol) of (±)-ethyl [{4-[(4-{[(2-carbamoylquinolin-4-yl)carbonyl]amino}-3,5-dimethyl-1H-pyrazol-1-yl)methyl]phenyl}(methyl)oxido-λ⁶-sulfanylidene]carbamate (example 376) in 4.2 mL ethanol was added 551 μL of a 21% sodium ethylate solution in ethanol. This reaction mixture was stirred at 60° C. for one hour and after cooling to room temperature 50 ml of water was added. This mixture was extracted twotimes with ethyl acetate. The combined organic phases were washed with saturated aq. sodium bicarbonate, brine, dried over sodium sulfate, filtered and evaporated to dryness. The crude product was purified via a Biotage chromatography system (25 g snap KP-Sil column, hexane/0-100% ethyl acetate, then ethyl acetate/0-100% methanol) to give 110 mg (53%) of the desired title compound.

¹H-NMR (400 MHz, DMSO d₆) δ (ppm)=2.15 (s, 3H), 2.20 (s, 3H), 3.04 (d, 3H), 4.17 (s, 1H), 5.36 (s, 2H), 7.38 (d, 2H), 7.77-7.83 (m, 1H), 7.87-7.96 (m, 4H), 8.20 (d, 1H), 8.24 (d, 1H), 8.27 (s, 1H), 8.37 (d, 1H), 10.09 (s, 1H).

### Example 378 and Example 379

### (S or R)-7-fluoro-N⁴-[5-methyl-1-(1-phenylethyl)-3-(trifluoromethyl)-1H-pyrazol-4-yl]quinoline-2,4-dicarboxamide and (R or S)-7-fluoro-N⁴-[5-methyl-1-(1-phenylethyl)-3-(trifluoromethyl)-1H-pyrazol-4-yl]quinoline-2,4-dicarboxamide

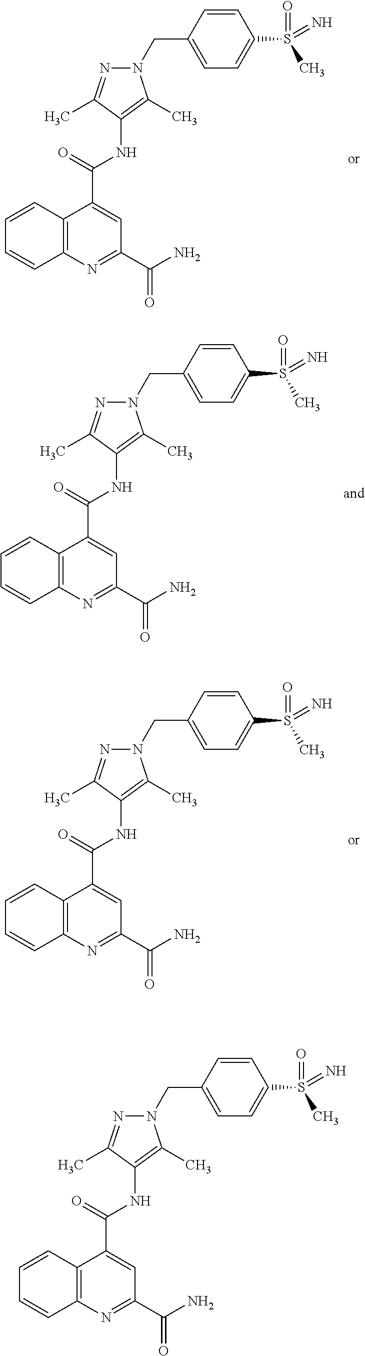 [SMILES: Cc1nn(Cc2ccc([S@@](C)(=N)=O)cc2)c(C)c1NC(=O)c1cc(C(N)=O)nc2ccccc12 ; Cc1nn(Cc2ccc([S@](C)(=N)=O)cc2)c(C)c1NC(=O)c1cc(C(N)=O)nc2ccccc12 ; Cc1nn(Cc2ccc([S@](C)(=N)=O)cc2)c(C)c1NC(=O)c1cc(C(N)=O)nc2ccccc12 ; Cc1nn(Cc2ccc([S@@](C)(=N)=O)cc2)c(C)c1NC(=O)c1cc(C(N)=O)nc2ccccc12]

110 mg of the racemic mixture of the title compound from example 377 was separated via a chiral HPLC (method 10) to give 23 mg of example 378 (Rt 7.8-8.9 min.) and 25 mg (Rt 9.0-10.1 min.) of example 379.

### Example 380

### 2-bromo-N-[1-(4-cyanobenzyl)-3,5-dimethyl-1H-pyrazol-4-yl]quinoline-4-carboxamide

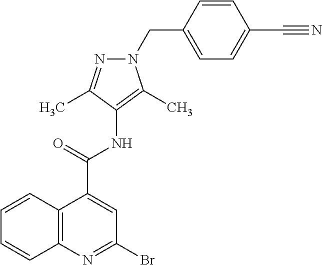 [SMILES: Cc1nn(Cc2ccc(C#N)cc2)c(C)c1NC(=O)c1cc(Br)nc2ccccc12]

In analogy to example 118), 898 mg (3.97 mmol) 4-[(4-amino-3,5-dimethyl-1H-pyrazol-1-yl)methyl]benzonitrile (intermediate 8C) and 1.00 g (3.97 mmol) 2-bromoquinoline-4-carboxylic acid were reacted to give after three subsequent purification via a Biotage chromatography system (25 g snap KP-Sil column, hexane/0-100% ethyl acetate, then ethyl acetate/0-100% methanol) 1.69 g (93%) of the desired title compound.

¹H-NMR (300 MHz, DMSO d₆) δ (ppm)=2.14 (s, 3H), 2.16 (s, 3H), 5.37 (s, 2H), 7.30 (d, 2H), 7.76 (ddd, 1H), 7.81-7.93 (m, 3H), 7.95 (s, 1H), 8.06 (d, 1H), 8.15 (d, 1H), 10.06 (s, 1H).

### Example 381

### N-[1-(4-cyanobenzyl)-3,5-dimethyl-1H-pyrazol-4-yl]-2-(methylsulfanyl)quinoline-4-carboxamide

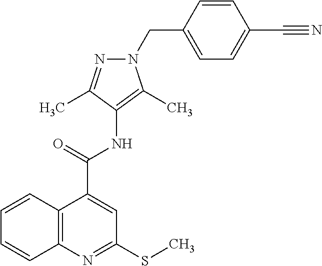 [SMILES: CSc1cc(C(=O)Nc2c(C)nn(Cc3ccc(C#N)cc3)c2C)c2ccccc2n1]

In analogy to example 118), 124 mg (0.557 mmol) 4-[(4-amino-3,5-dimethyl-1H-pyrazol-1-yl)methyl]benzonitrile (intermediate 8C) and 100 mg (0.46 mmol) 2-(methylsulfanyl)quinoline-4-carboxylic acid (prepared according to U.S. Pat. No. 6,699,879, 2004; page column 17-18) were reacted to give a solid. After filtration the solid was washed with THF, then dried to yield 103 mg (49%) of a white solid as the desired title compound.

¹H-NMR (400 MHz, DMSO d₆) δ (ppm)=2.15 (s, 3H), 2.17 (s, 3H), 2.70 (s, 3H), 5.38 (s, 2H), 7.32 (d, 2H), 7.55-7.63 (m, 2H), 7.75-7.80 (m, 1H), 7.85 (d, 2H), 7.96 (d, 1H), 8.08 (d, 1H), 9.96 (s, 1H).

### Example 382

### N-[1-(4-cyanobenzyl)-5-methyl-3-(trifluoromethyl)-1H-pyrazol-4-yl]-2-(methylsulfanyl)quinoline-4-carboxamide

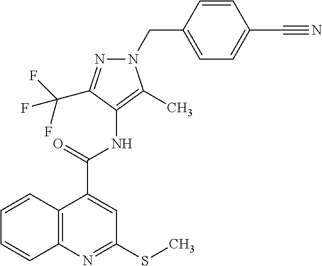 [SMILES: CSc1cc(C(=O)Nc2c(C(F)(F)F)nn(Cc3ccc(C#N)cc3)c2C)c2ccccc2n1]

In analogy to example 118), 1.41 g (5.02 mmol) 4-{[4-amino-5-methyl-3-(trifluoromethyl)-1H-pyrazol-1-yl]methyl}benzonitrile (intermediate 26C) and 1.00 g (4.56 mmol) 2-(methylsulfanyl)quinoline-4-carboxylic acid (prepared according to U.S. Pat. No. 6,699,879, 2004; page column 17-18) were reacted to give after two subsequent purification via a Biotage chromatography system (50 g then 25 g snap KP-Sil column, hexane/0-100% ethyl acetate, then ethyl acetate/0-80% methanol) 1.25 g (54%) of the desired title compound.

¹H-NMR (400 MHz, DMSO d₆) δ (ppm)=2.26 (s, 3H), 2.69 (s, 3H), 5.59 (s, 2H), 7.37 (d, 2H), 7.54 (s, 1H), 7.57 (ddd, 1H), 7.77 (ddd, 1H), 7.88 (d, 2H), 7.96 (d, 1H), 8.01 (d, 1H), 10.28 (s, 1H).

### Example 383 and Example 384

### (±)-N-[1-(4-cyanobenzyl)-5-methyl-3-(trifluoromethyl)-1H-pyrazol-4-yl]-2-(methylsulfinyl)quinoline-4-carboxamide and N-[1-(4-cyanobenzyl)-5-methyl-3-(trifluoromethyl)-1H-pyrazol-4-yl]-2-(methylsulfonyl)quinoline-4-carboxamide

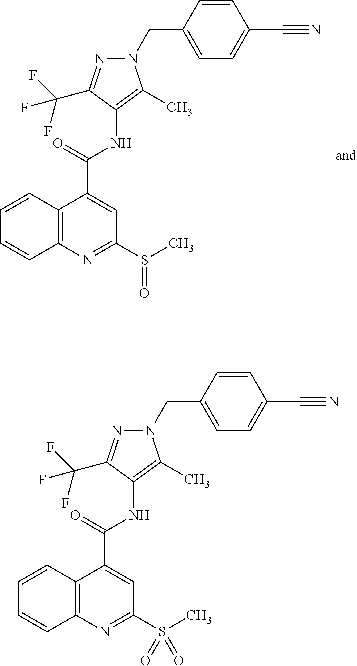 [SMILES: Cc1c(NC(=O)c2cc(S(C)=O)nc3ccccc23)c(C(F)(F)F)nn1Cc1ccc(C#N)cc1 ; Cc1c(NC(=O)c2cc(S(C)(=O)=O)nc3ccccc23)c(C(F)(F)F)nn1Cc1ccc(C#N)cc1]

To a stirred solution of 150 mg (0.31 mmol) of N-[1-(4-cyanobenzyl)-5-methyl-3-(trifluoromethyl)-1H-pyrazol-4-yl]-2-(methylsulfanyl)quinoline-4-carboxamide (example 382) in 5.0 mL dichloromethane was given portionwise 108 mg (0.62 mmol) meta-chloroperoxybenzoic acid (MCPBA) at room temperature. Then the mixture was stirred for an additional two hours at this temperature. After diluting the mixture with 30 mL water and separation of the organic phase, the aqueous phase was extracted three time with dichloromethane. Then the combined organic phases were washed three times with 50 mL saturated aq. NaHSO3-solution, dried over sodium sulfate, filtered and evaporated to dryness. This raw material was purified via preparative HPLC (method 5c) to yield 32 mg (19%) of (±)-N-[1-(4-cyanobenzyl)-5-methyl-3-(trifluoromethyl)-1H-pyrazol-4-yl]-2-(methylsulfinyl)quinoline-4-carboxamide and 41 mg (24%) of N-[1-(4-cyanobenzyl)-5-methyl-3-(trifluoromethyl)-1H-pyrazol-4-yl]-2-(methylsulfonyl)quinoline-4-carboxamide as the desired title compounds.

### NMR of (±)-N-[1-(4-cyanobenzyl)-5-methyl-3-(trifluoromethyl)-1H-pyrazol-4-yl]-2-(methylsulfinyl)quinoline-4-carboxamide as example 383

¹H-NMR (400 MHz, DMSO d₆) δ (ppm)=2.30 (s, 3H), 3.49 (s, 3H), 5.62 (s, 2H), 7.40 (d, 2H), 7.88-7.92 (m, 2H), 7.93-7.97 (m, 1H), 8.06 (ddd, 1H), 8.20-8.27 (m, 2H), 8.31 (d, 1H), 10.57 (s, 1H).

### NMR of N-[1-(4-cyanobenzyl)-5-methyl-3-(trifluoromethyl)-1H-pyrazol-4-yl]-2-(methylsulfonyl)quinoline-4-carboxamide as example 384

¹H-NMR (400 MHz, DMSO d₆) δ (ppm)=2.29 (s, 3H), 2.96 (s, 3H), 5.63 (s, 2H), 7.40 (d, 2H), 7.85 (ddd, 1H), 7.89-7.93 (m, 2H), 7.98 (ddd, 1H), 8.17 (s, 1H), 8.19 (d, 1H), 8.23 (d, 1H), 10.52 (s, 1H).

### Example 385

### (±)-N-[1-(4-cyanobenzyl)-5-methyl-3-(trifluoromethyl)-1H-pyrazol-4-yl]-2-(N-cyano-S-methylsulfinimidoyl)quinoline-4-carboxamide

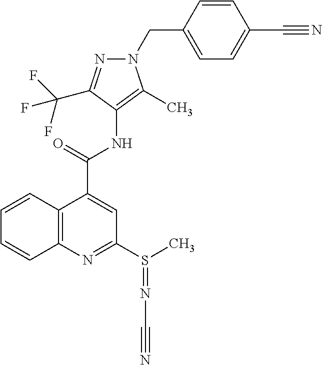 [SMILES: Cc1c(NC(=O)c2cc(S(C)=NC#N)nc3ccccc23)c(C(F)(F)F)nn1Cc1ccc(C#N)cc1]

To a stirred solution of 1.00 g (2.08 mmol) of N-[1-(4-cyanobenzyl)-5-methyl-3-(trifluoromethyl)-1H-pyrazol-4-yl]-2-(methylsulfanyl)quinoline-4-carboxamide (example 382) and 175 mg (4.15 mmol) cyanamide in 40 mL dichloromethane was added 736 mg (2.29 mmol) iodobenzene diacetete at 0° C. Then the mixture was stirred for an additional three hours at this temperature. After evaporation of the solvent the residue was purified via a Biotage chromatography system (10 g snap KP-Sil column, hexane/0-100% ethyl acetate, then ethyl acetate/0-80% methanol) to give 1.06 g (95%) of the desired title compound.

¹H-NMR (400 MHz, DMSO d₆) δ (ppm)=2.29 (s, 3H), 3.34 (s, 3H), 5.61 (s, 2H), 7.38 (d, 2H), 7.87-7.94 (m, 3H), 8.03 (ddd, 1H), 8.19-8.27 (m, 3H), 10.55 (s, 1H).

### Example 386

### (±)-N-[1-(4-cyanobenzyl)-5-methyl-3-(trifluoromethyl)-1H-pyrazol-4-yl]-2-(N-cyano-S-methylsulfonimidoyl)quinoline-4-carboxamide

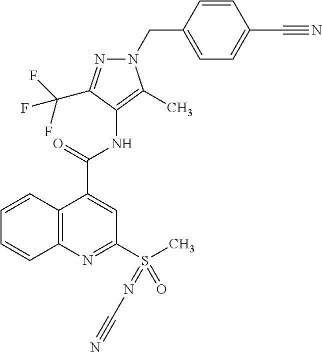 [SMILES: Cc1c(NC(=O)c2cc(S(C)(=O)=NC#N)nc3ccccc23)c(C(F)(F)F)nn1Cc1ccc(C#N)cc1]

To a stirred solution of 1.74 g (8.13 mmol) of sodium metaperiodate was added 92 mg (0.41 mmol) ruthenium (III) chloride hydrate in 15 mL dichloromethane followed by 1.06 g (2.03 mmol) of (±)-N-[1-(4-cyanobenzyl)-5-methyl-3-(trifluoromethyl)-1H-pyrazol-4-yl]-2-(N-cyano-S-methylsulfinimidoyl)quinoline-4-carboxamide (example 385) in 8 mL dichloromethane dropwise over a period of 5 minutes at room temperature. Then the mixture was stirred for three hours at this temperature. After filtration of the solid the liquid phase was diluted with 50 mL dichlormethane and 30 mL water. After extraction and separation of the organic phase, the aqueous phase was extracted again with dichloromethane. Then the combine organic phases were washed with brine, dried over sodium sulfate, filtered and evaporated to dryness. The obtained residue was purified via a Biotage chromatography system (25 g snap KP-Sil column, hexane/0-100% ethyl acetate, then ethyl acetate/0-100% methanol) to give 440 mg (37%) of the desired title compound.

¹H-NMR (400 MHz, DMSO d₆) δ (ppm)=2.32 (s, 3H), 3.92 (s, 3H), 5.63 (s, 2H), 7.39 (d, 2H), 7.89-7.93 (m, 2H), 8.03 (ddd, 1H), 8.13 (ddd, 1H), 8.28 (d, 1H), 8.35-8.40 (m, 2H), 10.67 (s, 1H).

### Example 387

### (±)-N-[1-(4-cyanobenzyl)-5-methyl-3-(trifluoromethyl)-1H-pyrazol-4-yl]-2-(S-methylsulfonimidoyl)quinoline-4-carboxamide

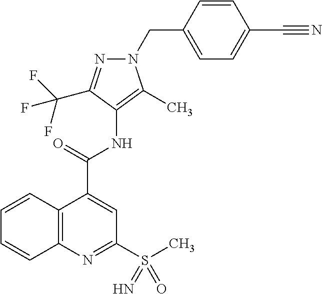 [SMILES: Cc1c(NC(=O)c2cc(S(C)(=N)=O)nc3ccccc23)c(C(F)(F)F)nn1Cc1ccc(C#N)cc1]

To a stirred solution of 440 mg (0.82 mmol) of (±)-N-[1-(4-cyanobenzyl)-5-methyl-3-(trifluoromethyl)-1H-pyrazol-4-yl]-2-(N-cyano-S-methylsulfonimidoyl)quinoline-4-carboxamide (example 386) in 47 mL dichloromethane was added at 0° C. 347 μL (2.46 mmol) trifluoroacetic acid anhydride. After stirring for 2 hours at room temperature the mixture was evaporated to dryness. The residue was taken up in 7.45 mL methanol and to this mixture was added 566 mg (4.09 mmol) potassium carbonate and stirring was continued for 2 hours at room temperature. The mixture was then diluted with brine and extracted twice with ethyl acetate. The combined organic phases were dried over sodium sulfate, filtered and evaporated to dryness. The obtained residue was purified via a Biotage chromatography system (10 g snap KP-Sil column, hexane/0-100% ethyl acetate, then ethyl acetate/0-90% methanol) to give 125 mg (28%) of the desired title compound.

¹H-NMR (400 MHz, DMSO d₆) δ (ppm)=2.24 (s, 3H), 3.33 (s, 3H), 5.60 (s, 2H), 6.68 (d, 1H), 7.25 (ddd, 1H), 7.36-7.42 (m, 3H), 7.58 (ddd, 1H), 7.70 (d, 1H), 7.88-7.92 (m, 2H), 10.27 (s, 1H), 12.04 (s, 1H).

### Example 388

### N⁴-(1-{4-[(dimethylamino)methyl]benzyl}-3,5-dimethyl-1H-pyrazol-4-yl)-7-fluoroquinoline-2,4-dicarboxamide

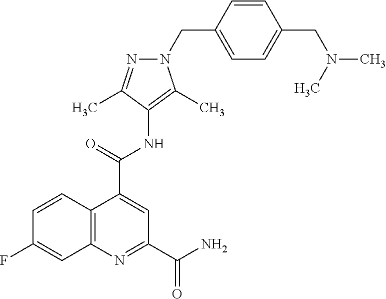 [SMILES: Cc1nn(Cc2ccc(CN(C)C)cc2)c(C)c1NC(=O)c1cc(C(N)=O)nc2cc(F)ccc12]

In analogy to example 118), 79 mg (0.30 mmol) 1-{4-[(dimethylamino)methyl]benzyl}-3,5-dimethyl-1H-pyrazol-4-amine (intermediate 71C) and 59 mg (0.25 mmol) 2-carbamoyl-7-fluoroquinoline-4-carboxylic acid (intermediate 37A) were reacted to give after a purification via preparative HPLC (method 5c) 26 mg (20%) of the desired title compound.

¹H-NMR (400 MHz, DMSO d₆) δ (ppm)=2.12 (s, 6H), 2.15 (s, 3H), 2.18 (s, 3H), 3.35 (s, 2H), 5.24 (s, 2H), 7.15 (d, 2H), 7.27 (d, 2H), 7.78 (ddd, 1H), 7.92 (dd, 1H), 7.96 (d, 1H), 8.28 (s, 1H), 8.36 (dd, 1H), 8.39 (d, 1H), 10.12 (s, 1H).

### Example 389

### N⁴-[1-(4-cyanobenzyl)-3-methyl-5-(trifluoromethyl)-1H-pyrazol-4-yl]-7-fluoroquinoline-2,4-dicarboxamide

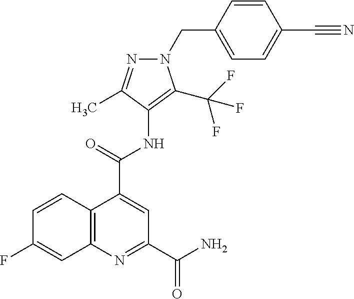 [SMILES: Cc1nn(Cc2ccc(C#N)cc2)c(C(F)(F)F)c1NC(=O)c1cc(C(N)=O)nc2cc(F)ccc12]

In analogy to example 118), 200 mg (0.71 mmol) 4-{[4-amino-3-methyl-5-(trifluoromethyl)-1H-pyrazol-1-yl]methyl}benzonitrile (intermediate 72C) and 139 mg (0.56 mmol) 2-carbamoyl-7-fluoroquinoline-4-carboxylic acid (intermediate 37A) were reacted to give after a purification via preparative HPLC 136 mg (46%) of the desired title compound.

¹H-NMR (400 MHz, DMSO d₆) δ (ppm)=2.25 (s, 3H), 5.60 (s, 2H), 7.34-7.38 (m, 2H), 7.81 (td, 1H), 7.87-7.91 (m, 2H), 7.94 (dd, 1H), 7.99 (d, 1H), 8.27 (s, 1H), 8.32 (dd, 1H), 8.41 (d, 1H), 10.64 (s, 1H).

### Example 390

### N⁴-[1-(4-cyano-2, 6-difluorobenzyl)-3,5-dimethyl-1H-pyrazol-4-yl]-7-fluoroquinoline-2,4-dicarboxamide

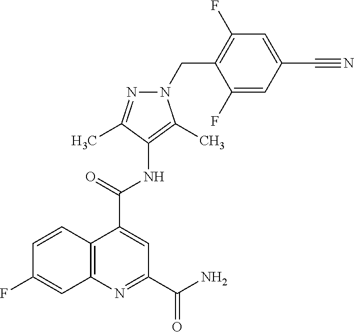 [SMILES: Cc1nn(Cc2c(F)cc(C#N)cc2F)c(C)c1NC(=O)c1cc(C(N)=O)nc2cc(F)ccc12]

In analogy to example 118), 101 mg (0.38 mmol) 4-[(4-amino-3,5-dimethyl-1H-pyrazol-1-yl)methyl]-3,5-difluorobenzonitrile (intermediate 73C) and 75 mg (0.32 mmol) 2-carbamoyl-7-fluoroquinoline-4-carboxylic acid (intermediate 37A) were reacted to give after a purification via preparative HPLC (method 3) 57 mg (36%) of the desired title compound.

¹H-NMR (400 MHz, DMSO d) δ (ppm)=2.06 (s, 3H), 2.32 (s, 3H), 5.32 (s, 2H), 7.78 (td, 1H), 7.84-7.90 (m, 2H), 7.92 (dd, 1H), 7.96 (d, 1H), 8.29 (s, 1H), 8.35 (dd, 1H), 8.40 (d, 1H), 10.12 (s, 1H).

### Example 391

### N⁴-[1-(4-cyano-2, 6-difluorobenzyl)-3,5-dimethyl-1H-pyrazol-4-yl]quinoline-2,4-dicarboxamide

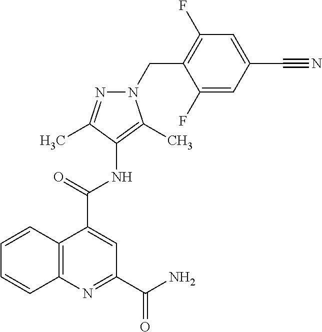 [SMILES: Cc1nn(Cc2c(F)cc(C#N)cc2F)c(C)c1NC(=O)c1cc(C(N)=O)nc2ccccc12]

In analogy to example 118), 109 mg (0.42 mmol) 4-[(4-amino-3,5-dimethyl-1H-pyrazol-1-yl)methyl]-3,5-difluorobenzonitrile (intermediate 73C) and 75 mg (0.35 mmol) 2-carbamoylquinoline-4-carboxylic acid (intermediate 4A) were reacted to give after a purification via preparative HPLC (method 3) 45 mg (27%) of the desired title compound.

¹H-NMR (400 MHz, DMSO d) δ (ppm)=2.07 (s, 3H), 2.33 (s, 3H), 5.32 (s, 2H), 7.82 (ddd, 1H), 7.85-7.90 (m, 2H), 7.91 (d, 1H), 7.95 (ddd, 1H), 8.22 (d, 1H), 8.26 (d, 1H), 8.29 (s, 1H), 8.40 (d, 1H), 10.07 (s, 1H).

### Example 392

### 6-chloro-N⁴-[1-(4-cyano-2, 6-difluorobenzyl)-3,5-dimethyl-1H-pyrazol-4-yl]-7-fluoroquinoline-2,4-dicarboxamide

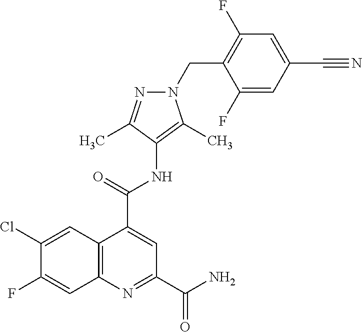 [SMILES: Cc1nn(Cc2c(F)cc(C#N)cc2F)c(C)c1NC(=O)c1cc(C(N)=O)nc2cc(F)c(Cl)cc12]

In analogy to example 118), 88 mg (0.34 mmol) 4-[(4-amino-3,5-dimethyl-1H-pyrazol-1-yl)methyl]-3,5-difluorobenzonitrile (intermediate 73C) and 75 mg (0.28 mmol) 2-carbamoyl-6-chloro-7-fluoroquinoline-4-carboxylic acid (intermediate 32A) were reacted to give after a purification via preparative HPLC (method 3) 17 mg (27%) of the desired title compound.

¹H-NMR (400 MHz, DMSO d₆) δ (ppm)=2.05 (s, 3H), 2.31 (s, 3H), 5.32 (s, 2H), 7.84-7.91 (m, 2H), 8.01 (d, 1H), 8.14 (d, 1H), 8.39 (s, 1H), 8.41 (d, 1H), 8.52 (d, 1H), 10.21 (s, 1H).

### Example 393

### N⁴-[1-(4-cyano-2, 6-difluorobenzyl)-3,5-dimethyl-1H-pyrazol-4-yl]-5-fluoroquinoline-2,4-dicarboxamide

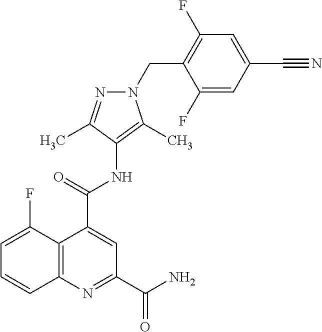 [SMILES: Cc1nn(Cc2c(F)cc(C#N)cc2F)c(C)c1NC(=O)c1cc(C(N)=O)nc2cccc(F)c12]

In analogy to example 118), 101 mg (0.38 mmol) 4-[(4-amino-3,5-dimethyl-1H-pyrazol-1-yl)methyl]-3,5-difluorobenzonitrile (intermediate 73C) and 75 mg (0.32 mmol) 2-carbamoyl-5-fluoroquinoline-4-carboxylic acid (intermediate 39A) were reacted to give after a purification via preparative HPLC (method 3) 55 mg (35%) of the desired title compound.

¹H-NMR (400 MHz, DMSO d) δ (ppm)=2.06 (s, 3H), 2.32 (s, 3H), 5.31 (s, 2H), 7.78 (td, 1H), 7.84-7.90 (m, 2H), 7.92 (dd, 1H), 7.97 (d, 1H), 8.29 (s, 1H), 8.35 (dd, 1H), 8.40 (d, 1H), 10.12 (s, 1H).

### Example 394

### 6-bromo-N⁴-[1-(4-cyano-2, 6-difluorobenzyl)-3,5-dimethyl-1H-pyrazol-4-yl]quinoline-2,4-dicarboxamide

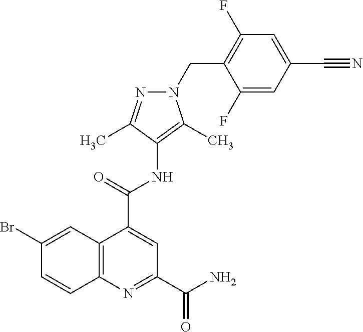 [SMILES: Cc1nn(Cc2c(F)cc(C#N)cc2F)c(C)c1NC(=O)c1cc(C(N)=O)nc2ccc(Br)cc12]

In analogy to example 118), 80 mg (0.31 mmol) 4-[(4-amino-3,5-dimethyl-1H-pyrazol-1-yl)methyl]-3,5-difluorobenzonitrile (intermediate 73C) and 75 mg (0.25 mmol) 6-bromo-2-carbamoylquinoline-4-carboxylic acid (intermediate 2A) were reacted to give after a purification via preparative HPLC (method 3) 19 mg (14%) of the desired title compound.

¹H-NMR (400 MHz, DMSO d₆) δ (ppm)=2.05 (s, 3H), 2.32 (s, 3H), 5.32 (s, 2H), 7.83-7.91 (m, 2H), 7.96 (d, 1H), 8.09 (dd, 1H), 8.15 (d, 1H), 8.37 (s, 1H), 8.43 (d, 1H), 8.48 (d, 1H), 10.17 (s, 1H).

### Example 395

### N⁴-[1-(4-cyano-2, 6-difluorobenzyl)-5-methyl-3-(trifluoromethyl)-1H-pyrazol-4-yl]-7-fluoroquinoline-2,4-dicarboxamide

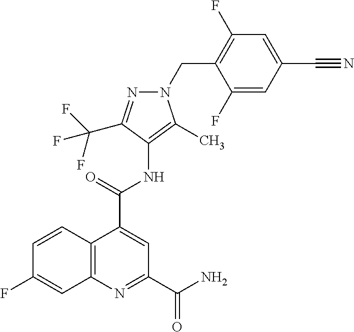 [SMILES: Cc1c(NC(=O)c2cc(C(N)=O)nc3cc(F)ccc23)c(C(F)(F)F)nn1Cc1c(F)cc(C#N)cc1F]

In analogy to example 118), 122 mg (0.38 mmol) 4-{[4-amino-5-methyl-3-(trifluoromethyl)-1H-pyrazol-1-yl]methyl}-3,5-difluorobenzonitrile (intermediate 74C) and 75 mg (0.32 mmol) 2-carbamoyl-7-fluoroquinoline-4-carboxylic acid (intermediate 37A) were reacted to give after a purification via preparative HPLC (method 3) 52 mg (30%) of the desired title compound.

¹H-NMR (400 MHz, DMSO d₆) δ (ppm)=2.40 (s, 3H), 5.57 (s, 2H), 7.81 (ddd, 1H), 7.89-7.96 (m, 3H), 7.98 (d, 1H), 8.26-8.32 (m, 2H), 8.41 (d, 1H), 10.47 (s, 1H).

### Example 396

### N⁴-[1-(4-cyano-2, 6-difluorobenzyl)-5-methyl-3-(trifluoromethyl)-1H-pyrazol-4-yl]quinoline-2,4-dicarboxamide

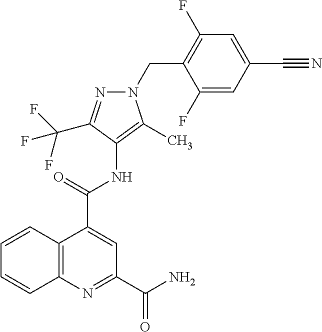 [SMILES: Cc1c(NC(=O)c2cc(C(N)=O)nc3ccccc23)c(C(F)(F)F)nn1Cc1c(F)cc(C#N)cc1F]

In analogy to example 118), 132 mg (0.42 mmol) 4-{[4-amino-5-methyl-3-(trifluoromethyl)-1H-pyrazol-1-yl]methyl}-3,5-difluorobenzonitrile (intermediate 74C) and 75 mg (0.35 mmol) 2-carbamoylquinoline-4-carboxylic acid (intermediate 4A) were reacted to give after a purification via preparative HPLC (method 3) 46 mg (25%) of the desired title compound.

¹H-NMR (400 MHz, DMSO d₆) δ (ppm)=2.40 (s, 3H), 5.57 (s, 2H), 7.83 (ddd, 1H), 7.89-8.00 (m, 4H), 8.17-8.30 (m, 3H), 8.42 (d, 1H), 10.41 (s, 1H).

### Example 397

### N⁴-[1-(4-cyano-2, 6-difluorobenzyl)-5-methyl-3-(trifluoromethyl)-1H-pyrazol-4-yl]-5-fluoroquinoline-2,4-dicarboxamide

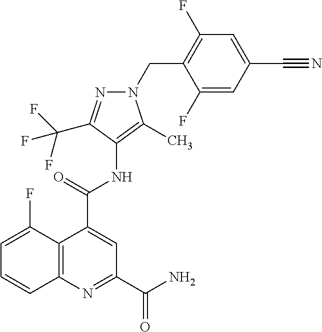 [SMILES: Cc1c(NC(=O)c2cc(C(N)=O)nc3cccc(F)c23)c(C(F)(F)F)nn1Cc1c(F)cc(C#N)cc1F]

In analogy to example 118), 122 mg (0.38 mmol) 4-{[4-amino-5-methyl-3-(trifluoromethyl)-1H-pyrazol-1-yl]methyl}-3,5-difluorobenzonitrile (intermediate 74C) and 75 mg (0.32 mmol) 2-carbamoyl-5-fluoroquinoline-4-carboxylic acid (intermediate 39A) were reacted to give after a purification via preparative HPLC (method 3) 39 mg (22%) of the desired title compound.

¹H-NMR (400 MHz, DMSO d₆) δ (ppm)=2.40 (s, 3H), 5.57 (s, 2H), 7.81 (ddd, 1H), 7.89-7.96 (m, 3H), 7.98 (d, 1H), 8.26-8.32 (m, 2H), 8.41 (d, 1H), 10.47 (s, 1H).

### Example 398

### 6-chloro-N⁴-[1-(4-cyano-2, 6-difluorobenzyl)-5-methyl-3-(trifluoromethyl)-1H-pyrazol-4-yl]-7-fluoroquinoline-2,4-dicarboxamide

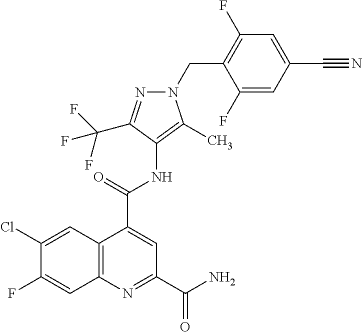 [SMILES: Cc1c(NC(=O)c2cc(C(N)=O)nc3cc(F)c(Cl)cc23)c(C(F)(F)F)nn1Cc1c(F)cc(C#N)cc1F]

In analogy to example 118), 106 mg (0.34 mmol) 4-{[4-amino-5-methyl-3-(trifluoromethyl)-1H-pyrazol-1-yl]methyl}-3,5-difluorobenzonitrile (intermediate 74C) and 75 mg (0.28 mmol) 2-carbamoyl-6-chloro-7-fluoroquinoline-4-carboxylic acid (intermediate 32A) were reacted to give after a purification via preparative HPLC (method 3) 55 mg (33%) of the desired title compound.

¹H-NMR (400 MHz, DMSO d₆) δ (ppm)=2.39 (s, 3H), 5.57 (s, 2H), 7.89-7.96 (m, 2H), 8.02 (d, 1H), 8.15 (d, 1H), 8.37 (s, 1H), 8.40-8.46 (m, 2H), 10.55 (s, 1H).

### Example 399

### 6-bromo-N⁴-[1-(4-cyano-2, 6-difluorobenzyl)-5-methyl-3-(trifluoromethyl)-1H-pyrazol-4-yl]quinoline-2,4-dicarboxamide

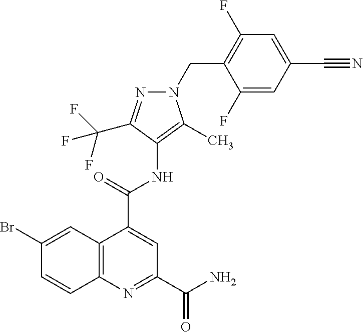 [SMILES: Cc1c(NC(=O)c2cc(C(N)=O)nc3ccc(Br)cc23)c(C(F)(F)F)nn1Cc1c(F)cc(C#N)cc1F]

In analogy to example 118), 96 mg (0.31 mmol) 4-{[4-amino-5-methyl-3-(trifluoromethyl)-1H-pyrazol-1-yl]methyl}-3,5-difluorobenzonitrile (intermediate 74C) and 75 mg (0.25 mmol) 6-bromo-2-carbamoylquinoline-4-carboxylic acid (intermediate 2A) were reacted to give after a purification via preparative HPLC (method 3) 58 mg (37%) of the desired title compound.

¹H-NMR (400 MHz, DMSO d₆) δ (ppm)=2.40 (s, 3H), 5.57 (s, 2H), 7.89-7.96 (m, 2H), 7.98 (d, 1H), 8.10 (dd, 1H), 8.16 (d, 1H), 8.36 (s, 1H), 8.41 (d, 1H), 8.44 (d, 1H), 10.50 (s, 1H).

### Example 400

### 7-fluoro-N⁴-{5-methyl-1-[4-(1H-tetrazol-5-yl)benzyl]-3-(trifluoromethyl)-1H-pyrazol-4-yl}quinoline-2,4-dicarboxamide

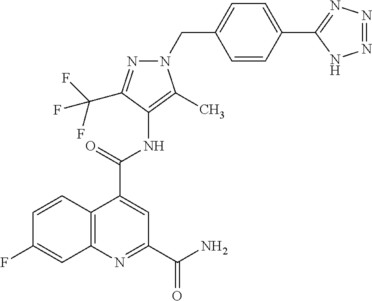 [SMILES: Cc1c(NC(=O)c2cc(C(N)=O)nc3cc(F)ccc23)c(C(F)(F)F)nn1Cc1ccc(-c2nnn[nH]2)cc1]

A mixture of 100 mg (0.20 mmol) N⁴-[1-(4-cyanobenzyl)-5-methyl-3-(trifluoromethyl)-1H-pyrazol-4-yl]-7-fluoroquinoline-2,4-dicarboxamide (example 240), 48 mg (0.75 mmol) sodium azide and 48 mg (0.91 mmol) ammonium chloride in 1.0 mL DMF was heated up to 115° C. for 3 hours. After cooling to room temperature 1M aq. hydrochloric acid was added carefully. The formed solid was isolated by filtration and purified via preparative HPLC (method 5c) to give 12 mg (10%) of the desired title compound.

¹H-NMR (400 MHz, DMSO d₆) δ (ppm)=2.31 (s, 3H), 5.56 (s, 2H), 7.39 (d, 2H), 7.80 (ddd, 1H), 7.93 (dd, 1H), 7.98 (d, 1H), 8.04 (d, 2H), 8.14 (s, 1H), 8.27 (s, 1H), 8.28-8.33 (m, 1H), 8.40 (d, 1H), 10.47 (s, 1H).

### Example 401

### 7-fluoro-N⁴-{1-[4-(methoxymethyl)benzyl]-5-methyl-3-(trifluoromethyl)-1H-pyrazol-4-yl}quinoline-2,4-dicarboxamide

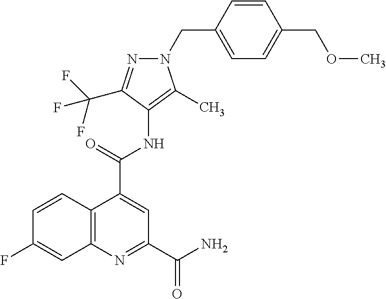 [SMILES: COCc1ccc(Cn2nc(C(F)(F)F)c(NC(=O)c3cc(C(N)=O)nc4cc(F)ccc34)c2C)cc1]

In analogy to example 118), 135 mg (0.45 mmol) 1-[4-(methoxymethyl)benzyl]-5-methyl-3-(trifluoromethyl)-1H-pyrazol-4-amine (intermediate 75C) and 88 mg (0.25 mmol) 2-carbamoyl-7-fluoroquinoline-4-carboxylic acid (intermediate 37A) were reacted to give after a purification via preparative HPLC (method 5e) 84 mg (42%) of the desired title compound.

¹H-NMR (400 MHz, DMSO d₆) δ (ppm)=2.26 (s, 3H), 3.27 (s, 3H), 4.39 (s, 2H), 5.46 (s, 2H), 7.22 (d, 2H), 7.33 (d, 2H), 7.78 (ddd, 1H), 7.91 (dd, 1H), 7.95 (d, 1H), 8.25 (s, 1H), 8.28 (dd, 1H), 8.37 (d, 1H), 10.42 (s, 1H).

### Example 402

### 5-fluoro-N⁴-{1-[4-(methoxymethyl)benzyl]-5-methyl-3-(trifluoromethyl)-1H-pyrazol-4-yl}quinoline-2,4-dicarboxamide

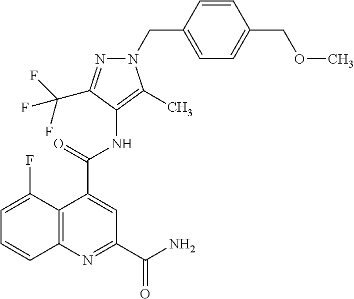 [SMILES: COCc1ccc(Cn2nc(C(F)(F)F)c(NC(=O)c3cc(C(N)=O)nc4cccc(F)c34)c2C)cc1]

In analogy to example 118), 135 mg (0.45 mmol) 1-[4-(methoxymethyl)benzyl]-5-methyl-3-(trifluoromethyl)-1H-pyrazol-4-amine (intermediate 75C) and 88 mg (0.25 mmol) 2-carbamoyl-5-fluoroquinoline-4-carboxylic acid (intermediate 39A) were reacted to give after a purification via preparative HPLC (method 5e) 82 mg (41%) of the desired title compound.

¹H-NMR (400 MHz, DMSO d₆) δ (ppm)=2.28 (s, 3H), 3.29 (s, 3H), 4.41 (s, 2H), 5.48 (s, 2H), 7.24 (d, 2H), 7.35 (d, 2H), 7.80 (ddd, 1H), 7.93 (dd, 1H), 7.98 (d, 1H), 8.27 (s, 1H), 8.30 (dd, 1H), 8.40 (d, 1H), 10.45 (s, 1H).

### Example 403

### N⁴-{1-[4-(cyanomethyl)benzyl]-5-methyl-3-(trifluoromethyl)-1H-pyrazol-4-yl}-7-fluoroquinoline-2,4-dicarboxamide

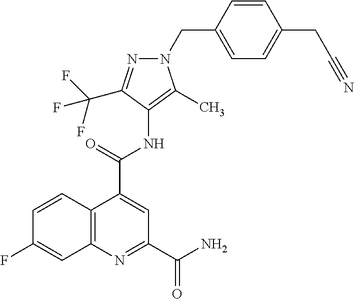 [SMILES: Cc1c(NC(=O)c2cc(C(N)=O)nc3cc(F)ccc23)c(C(F)(F)F)nn1Cc1ccc(CC#N)cc1]

In analogy to example 118), 53 mg (0.18 mmol) (4-{[4-amino-5-methyl-3-(trifluoromethyl)-1H-pyrazol-1-yl]methyl}phenyl)acetonitrile (intermediate 76C) and 35 mg (0.15 mmol) 2-carbamoyl-7-fluoroquinoline-4-carboxylic acid (intermediate 37A) were reacted to give after a purification via preparative HPLC (method 5e) 40 mg (50%) of the desired title compound.

¹H-NMR (400 MHz, DMSO d₆) δ (ppm)=2.28 (s, 3H), 4.05 (s, 2H), 5.49 (s, 2H), 7.29 (d, 2H), 7.39 (d, 2H), 7.80 (ddd, 1H), 7.93 (dd, 1H), 7.98 (d, 1H), 8.26-8.27 (m, 1H), 8.30 (dd, 1H), 8.41 (d, 1H), 10.45 (s, 1H).

### Example 404

### 6-bromo-N-(3,5-dimethyl-1-{[5-(methylcarbamoyl)-1,2,4-oxadiazol-3-yl]methyl}-1H-pyrazol-4-yl)-2-(trifluoromethyl)quinoline-4-carboxamide

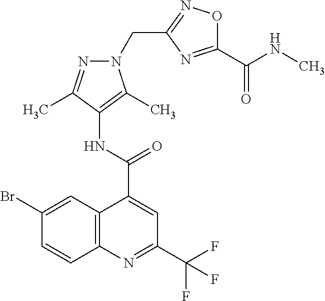 [SMILES: CNC(=O)c1nc(Cn2nc(C)c(NC(=O)c3cc(C(F)(F)F)nc4ccc(Br)cc34)c2C)no1]

In analogy to example 118), 100 mg (0.32 mmol) 3-[(4-amino-3,5-dimethyl-1H-pyrazol-1-yl)methyl]-N-methyl-1,2,4-oxadiazole-5-carboxamide (intermediate 77C) and 85 mg (0.27 mmol) 6-bromo-2-(trifluoromethyl)quinoline-4-carboxylic acid (intermediate 1A) were reacted to give after a purification via preparative HPLC (method 3) 85 mg (47%) of the desired title compound.

¹H NMR (400 MHz, DMSO d₆): δ (ppm)=2.13 (s, 3H), 2.31 (s, 3H), 2.79 (d, 3H), 5.53 (s, 2H), 8.15 (dd, 1H), 8.24 (d, 1H), 8.30 (s, 1H), 8.49 (d, 1H), 9.32 (br. q., 1H), 10.21 (s, 1H).

### Example 405

### tert-butyl 4-[(4-{[(2-carbamoylquinolin-4-yl)carbonyl]amino}-3,5-dimethyl-1H-pyrazol-1-yl)methyl]piperidine-1-carboxylate

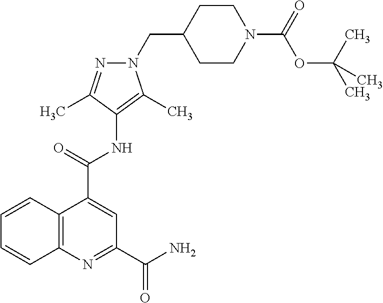 [SMILES: Cc1nn(CC2CCN(C(=O)OC(C)(C)C)CC2)c(C)c1NC(=O)c1cc(C(N)=O)nc2ccccc12]

In analogy to example 118), 1.58 g (4.61 mmol) tert-butyl 4-[(4-amino-3,5-dimethyl-1H-pyrazol-1-yl)methyl]piperidine-1-carboxylate (intermediate 78C) and 1.04 g (3.84 mmol) 2-carbamoylquinoline-4-carboxylic acid (intermediate 4A) were reacted to give after a purification via preparative HPLC (method 3) 1.34 g (65%) of the desired title compound.

¹H-NMR (300 MHz, DMSO d₆) δ (ppm)=1.11 (dddd, 2H), 1.39 (s, 9H), 1.51 (br. d., 2H), 2.00 (m, 1H), 2.13 (s, 3H), 2.21 (s, 3H), 2.65 (m, 2H), 3.87 (d, 2H), 3.95 (br. d., 2H), 7.82 (br. dd., 1H), 7.89 (br. s., 1H), 7.94 (br. dd., 1H), 8.21 (br. d., 1H), 8.26 (br. d., 1H), 8.27 (s, 1H), 8.38 (br. s., 1H), 10.01 (s, 1H).

### Example 406

### N⁴-[3,5-dimethyl-1-(piperidin-4-ylmethyl)-1H-pyrazol-4-yl]quinoline-2,4-dicarboxamide

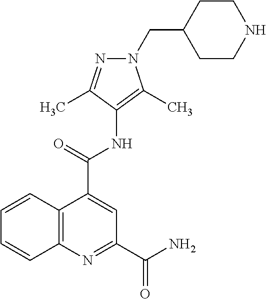 [SMILES: Cc1nn(CC2CCNCC2)c(C)c1NC(=O)c1cc(C(N)=O)nc2ccccc12]

A solution of 1.34 g (2.65 mmol) tert-butyl 4-[(4-{[(2-carbamoylquinolin-4-yl)carbonyl]amino}-3,5-dimethyl-1H-pyrazol-1-yl)methyl]piperidine-1-carboxylate (example 405) in 18 mL dichloromethane was stirred with 2.04 mL (26.5 mmol) trifluoroacetic acid for 70 hours. The reaction mixture was filtered over NH₂ derivatized silica gel, and the filtrate was evaporated yielding 1.29 g (96%) of the desired title compound.

¹H-NMR (300 MHz, DMSO d₆) δ (ppm)=1.40 (dddd, 2H), 1.72 (br. d., 2H), 2.11 (m, 1H), 2.13 (s, 3H), 2.23 (s, 3H), 2.88 (m, 2H), 3.29 (br. d., 2H), 3.93 (d, 2H), 7.82 (br. dd., 1H), 7.92 (br. s., 1H), 7.95 (br. dd., 1H), 8.21 (br. d., 1H), 8.24 (br. d., 1H), 8.28 (s, 1H), 8.41 (br. s., 1H), 10.06 (s, 1H).

### Example 407

### N⁴-(1-{[1-(ethylsulfonyl)piperidin-4-yl]methyl}-3,5-dimethyl-1H-pyrazol-4-yl)quinoline-2,4-dicarboxamide

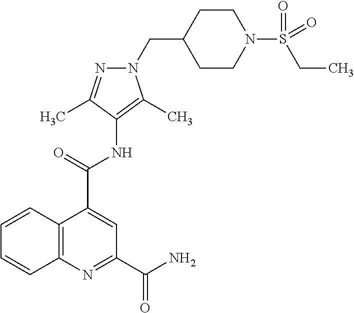 [SMILES: CCS(=O)(=O)N1CCC(Cn2nc(C)c(NC(=O)c3cc(C(N)=O)nc4ccccc34)c2C)CC1]

A solution of 100 mg (246 μmol) N⁴-[3,5-dimethyl-1-(piperidin-4-ylmethyl)-1H-pyrazol-4-yl]quinoline-2,4-dicarboxamide (example 406) in 3 mL DMF was stirred with 32.6 μL (344 μmol) ethanesulfonyl chloride and 206 μL (1.48 mmol) triethylamine overnight. Saturated aqueous sodium bicarbonate and ethyl acetate were added to the reaction. The mixture was extracted with butanol, and the combined organic phase was washed with brine, dried, filtered, and evaporated. Purification by preparative HPLC (method 3) yielded 58 mg (47%) of the desired title compound.

¹H-NMR (300 MHz, DMSO d₆) δ (ppm)=1.20 (t, 3H), 1.27 (m, 2H), 1.61 (br. d., 2H), 1.98 (m, 1H), 2.13 (s, 3H), 2.22 (s, 3H), 2.77 (m, 2H), 3.01 (q, 2H), 3.60 (br. d., 2H), 3.91 (d, 2H), 7.82 (br. dd., 1H), 7.92 (br. s., 1H), 7.95 (br. dd., 1H), 8.21 (br. d., 1H), 8.25 (br. d., 1H), 8.27 (s, 1H), 8.41 (br. s., 1H), 10.04 (s, 1H).

### Example 408

### 6-chloro-7-fluoro-N⁴-{5-methyl-1-[(5-methyl-1,2-oxazol-3-yl)methyl]-3-(trifluoromethyl)-1H-pyrazol-4-yl}quinoline-2,4-dicarboxamide

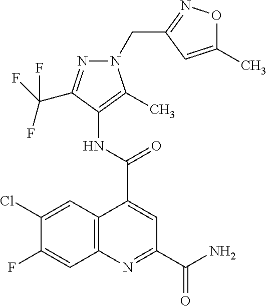 [SMILES: Cc1cc(Cn2nc(C(F)(F)F)c(NC(=O)c3cc(C(N)=O)nc4cc(F)c(Cl)cc34)c2C)no1]

In analogy to example 118), 100 mg (0.38 mmol) 5-methyl-1-[(5-methyl-1,2-oxazol-3-yl)methyl]-3-(trifluoromethyl)-1H-pyrazol-4-amine (intermediate 79C) and 86 mg (0.32 mmol) 2-carbamoyl-6-chloro-7-fluoroquinoline-4-carboxylic acid (intermediate 32A) were reacted to give after a purification via preparative HPLC (method 4) 18 mg (9%) of the desired title compound.

¹H NMR (400 MHz, DMSO d₆): δ (ppm)=2.32 (s, 3H), 2.41 (s, 3H), 5.54 (s, 2H), 6.21 (s, 1H), 8.02 (br. s., 1H), 8.15 (d, 1H), 8.36 (s, 1H), 8.41 (br. s., 1H), 8.43 (d, 1H), 10.54 (s, 1H).

### Example 409

### 6-chloro-N⁴-{1-[(5-ethyl-1,2,4-oxadiazol-3-yl)methyl]-5-methyl-3-(trifluoromethyl)-1H-pyrazol-4-yl}-7-fluoroquinoline-2,4-dicarboxamide

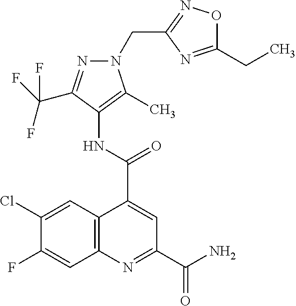 [SMILES: CCc1nc(Cn2nc(C(F)(F)F)c(NC(=O)c3cc(C(N)=O)nc4cc(F)c(Cl)cc34)c2C)no1]

In analogy to example 118), 100 mg (0.33 mmol) 1-[(5-ethyl-1,2,4-oxadiazol-3-yl)methyl]-5-methyl-3-(trifluoromethyl)-1H-pyrazol-4-amine (intermediate 80C) and 92 mg (0.27 mmol) 2-carbamoyl-6-chloro-7-fluoroquinoline-4-carboxylic acid (intermediate 32A) were reacted to give after a purification via preparative HPLC (method 4) 30 mg (17%) of the desired title compound.

¹H NMR (400 MHz, DMSO d₆): δ (ppm)=1.28 (t, 3H), 2.36 (s, 3H), 2.96 (q, 2H), 5.68 (s, 2H), 8.02 (br. s., 1H), 8.15 (d, 1H), 8.37 (s, 1H), 8.41 (br. s., 1H), 8.43 (d, 1H), 10.57 (s, 1H).

### Example 410

### 6-chloro-N⁴-{1-[(3-ethyl-1,2-oxazol-5-yl)methyl]-5-methyl-3-(trifluoromethyl)-1H-pyrazol-4-yl}-7-fluoroquinoline-2,4-dicarboxamide

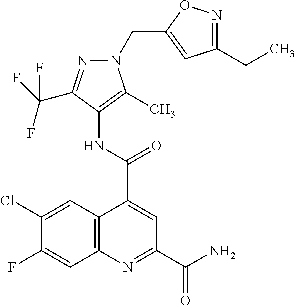 [SMILES: CCc1cc(Cn2nc(C(F)(F)F)c(NC(=O)c3cc(C(N)=O)nc4cc(F)c(Cl)cc34)c2C)on1]

In analogy to example 118), 90 mg (0.33 mmol) 1-[(3-ethyl-1,2-oxazol-5-yl)methyl]-5-methyl-3-(trifluoromethyl)-1H-pyrazol-4-amine (intermediate 81C) and 73.5 mg (0.27 mmol) 2-carbamoyl-6-chloro-7-fluoroquinoline-4-carboxylic acid (intermediate 32A) were reacted to give after a purification via preparative HPLC (method 3) 46 mg (27%) of the desired title compound.

¹H NMR (400 MHz, DMSO d₆): δ (ppm)=1.19 (t, 3H), 2.37 (s, 3H), 2.64 (q, 2H), 5.68 (s, 2H), 6.50 (s, 1H), 8.02 (br. s., 1H), 8.15 (d, 1H), 8.37 (s, 1H), 8.41 (br. s., 1H), 8.43 (d, 1H), 10.56 (s, 1H).

### Example 411

### 6-chloro-N⁴-{3,5-dimethyl-1-[(3-methyl-1,2-oxazol-5-yl)methyl]-1H-pyrazol-4-yl}-7-fluoroquinoline-2,4-dicarboxamide

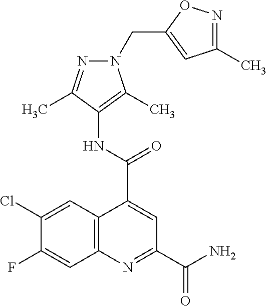 [SMILES: Cc1cc(Cn2nc(C)c(NC(=O)c3cc(C(N)=O)nc4cc(F)c(Cl)cc34)c2C)on1]

In analogy to example 118), 150 mg (0.73 mmol) 3,5-dimethyl-1-[(3-methyl-1,2-oxazol-5-yl)methyl]-1H-pyrazol-4-amine (intermediate 82C) and 163 mg (0.61 mmol) 2-carbamoyl-6-chloro-7-fluoroquinoline-4-carboxylic acid (intermediate 32A) were reacted to give after a purification via preparative HPLC (method 3) 77 mg (22%) of the desired title compound.

¹H NMR (400 MHz, DMSO d₆): δ (ppm)=2.11 (s, 3H), 2.22 (s, 3H), 2.26 (s, 3H), 5.41 (s, 2H), 6.28 (s, 1H), 7.99 (br. s., 1H), 8.13 (d, 1H), 8.39 (s, 1H), 8.39 (br. s., 1H), 8.52 (d, 1H), 10.22 (s, 1H).

### Example 412

### 6-chloro-N⁴-{3,5-dimethyl-1-[(5-methyl-1,2-oxazol-3-yl)methyl]-1H-pyrazol-4-yl}-7-fluoroquinoline-2,4-dicarboxamide

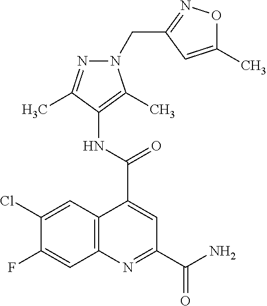 [SMILES: Cc1cc(Cn2nc(C)c(NC(=O)c3cc(C(N)=O)nc4cc(F)c(Cl)cc34)c2C)no1]

In analogy to example 118), 92 mg (0.45 mmol) 3,5-dimethyl-1-[(5-methyl-1,2-oxazol-3-yl)methyl]-1H-pyrazol-4-amine (intermediate 83C) and 100 mg (0.37 mmol) 2-carbamoyl-6-chloro-7-fluoroquinoline-4-carboxylic acid (intermediate 32A) were reacted to give after a purification via preparative HPLC (method 4) 40 mg (19%) of the desired title compound.

¹H NMR (400 MHz, DMSO d₆): δ (ppm)=2.11 (s, 3H), 2.22 (s, 3H), 2.38 (s, 3H), 5.28 (s, 2H), 6.09 (s, 1H), 8.00 (br. s., 1H), 8.13 (d, 1H), 8.38 (s, 1H), 8.40 (br. s., 1H), 8.51 (d, 1H), 10.22 (s, 1H).

### Example 413

### 6-chloro-N⁴-[1-{[1-(ethylsulfonyl)piperidin-4-yl]methyl}-5-methyl-3-(trifluoromethyl)-1H-pyrazol-4-yl]-7-fluoroquinoline-2,4-dicarboxamide

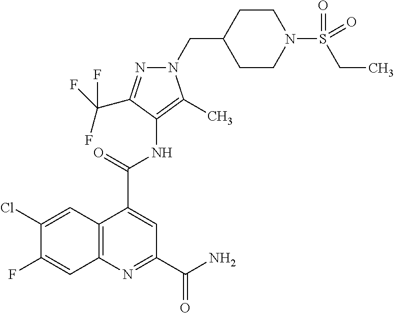 [SMILES: CCS(=O)(=O)N1CCC(Cn2nc(C(F)(F)F)c(NC(=O)c3cc(C(N)=O)nc4cc(F)c(Cl)cc34)c2C)CC1]

In analogy to example 118), 120 mg (0.31 mmol) 1-{[1-(ethylsulfonyl)piperidin-4-yl]methyl}-5-methyl-3-(trifluoromethyl)-1H-pyrazol-4-amine (intermediate 86C) and 85 mg (0.25 mmol) 2-carbamoyl-6-chloro-7-fluoroquinoline-4-carboxylic acid (intermediate 32A) were reacted to give after a purification via preparative HPLC (method 3) 81 mg (44%) of the desired title compound.

¹H-NMR (400 MHz, DMSO d₆) δ (ppm)=1.21 (t, 3H), 1.31 (m, 2H), 1.61 (br. d., 2H), 2.05 (m, 1H), 2.31 (s, 3H), 2.81 (m, 2H), 3.02 (q, 2H), 3.62 (br. d., 2H), 4.13 (d, 2H), 8.00 (br. s., 1H), 8.15 (d, 1H), 8.36 (s, 1H), 8.40 (br. s., 1H), 8.43 (d, 1H), 10.49 (s, 1H).

### Example 414

### 6-chloro-N⁴-{1-[(3-ethyl-1,2-oxazol-5-yl)methyl]-3,5-dimethyl-1H-pyrazol-4-yl}-7-fluoroquinoline-2,4-dicarboxamide

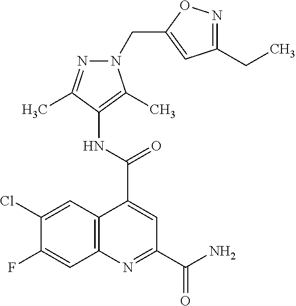 [SMILES: CCc1cc(Cn2nc(C)c(NC(=O)c3cc(C(N)=O)nc4cc(F)c(Cl)cc34)c2C)on1]

In analogy to example 118), 90 mg (0.38 mmol) 1-[(3-ethyl-1,2-oxazol-5-yl)methyl]-3,5-dimethyl-1H-pyrazol-4-amine (intermediate 87C) and 84.1 mg (0.31 mmol) 2-carbamoyl-6-chloro-7-fluoroquinoline-4-carboxylic acid (intermediate 32A) were reacted to give after a purification via preparative HPLC (method 3) 39 mg (21%) of the desired title compound.

¹H NMR (400 MHz, DMSO d₆): δ (ppm)=1.18 (t, 3H), 2.11 (s, 3H), 2.27 (s, 3H), 2.61 (q, 2H), 5.41 (s, 2H), 6.35 (s, 1H), 7.99 (br. s., 1H), 8.13 (d, 1H), 8.39 (s, 1H), 8.39 (br. s., 1H), 8.52 (d, 1H), 10.23 (s, 1H).

### Example 415

### 6-chloro-N⁴-(1-{[1-(ethylsulfonyl)piperidin-4-yl]methyl}-3,5-dimethyl-1H-pyrazol-4-yl)-7-fluoroquinoline-2,4-dicarboxamide

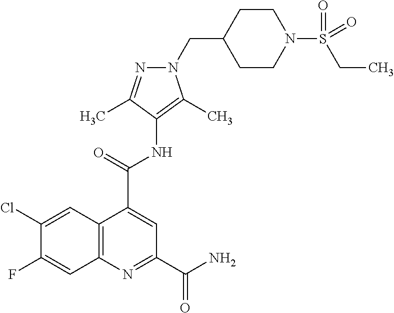 [SMILES: CCS(=O)(=O)N1CCC(Cn2nc(C)c(NC(=O)c3cc(C(N)=O)nc4cc(F)c(Cl)cc34)c2C)CC1]

In analogy to example 118), 100 mg (0.31 mmol) 1-{[1-(ethylsulfonyl)piperidin-4-yl]methyl}-3,5-dimethyl-1H-pyrazol-4-amine (intermediate 88C) and 72 mg (0.26 mmol) 2-carbamoyl-6-chloro-7-fluoroquinoline-4-carboxylic acid (intermediate 32A) were reacted to give after a purification via preparative HPLC (method 3) 51 mg (29%) of the desired title compound.

¹H-NMR (400 MHz, DMSO d₆) δ (ppm)=1.20 (t, 3H), 1.27 (dddd, 2H), 1.61 (br. d., 2H), 1.99 (m, 1H), 2.11 (s, 3H), 2.20 (s, 3H), 2.78 (m, 2H), 3.02 (q, 2H), 3.61 (br. d., 2H), 3.91 (d, 2H), 7.99 (br. s., 1H), 8.13 (d, 1H), 8.38 (s, 1H), 8.39 (br. s., 1H), 8.52 (d, 1H), 10.17 (s, 1H).

### Example 416

### 6-chloro-N⁴-(3,5-dimethyl-1-{[3-(propan-2-yl)-1,2-oxazol-5-yl]methyl}-1H-pyrazol-4-yl)-7-fluoroquinoline-2,4-dicarboxamide

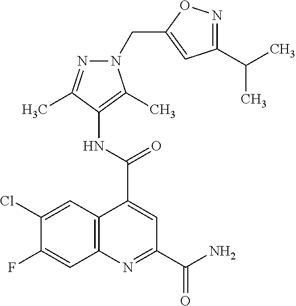 [SMILES: Cc1nn(Cc2cc(C(C)C)no2)c(C)c1NC(=O)c1cc(C(N)=O)nc2cc(F)c(Cl)cc12]

In analogy to example 118), 100 mg (0.41 mmol) 3,5-dimethyl-1-{[3-(propan-2-yl)-1,2-oxazol-5-yl]methy}-1H-pyrazol-4-amine (intermediate 89C) and 90.8 mg (0.34 mmol) 2-carbamoyl-6-chloro-7-fluoroquinoline-4-carboxylic acid (intermediate 32A) were reacted to give after a purification via preparative HPLC (method 3) 38 mg (18%) of the desired title compound.

¹H NMR (400 MHz, DMSO d₆): δ (ppm)=1.21 (d, 6H), 2.11 (s, 3H), 2.27 (s, 3H), 2.99 (sept, 1H), 5.41 (s, 2H), 6.41 (s, 1H), 7.99 (br. s., 1H), 8.14 (d, 1H), 8.38 (br. s., 1H), 8.39 (s, 1H), 8.52 (d, 1H), 10.22 (s, 1H).

### Example 417

### 6-chloro-N⁴-{1-[(5-cyclopropyl-1,2-oxazol-3-yl)methyl]-3,5-dimethyl-1H-pyrazol-4-yl}-7-fluoroquinoline-2,4-dicarboxamide

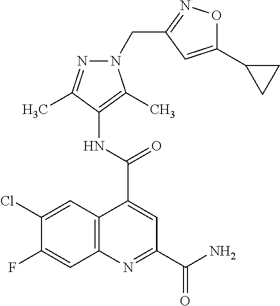 [SMILES: Cc1nn(Cc2cc(C3CC3)on2)c(C)c1NC(=O)c1cc(C(N)=O)nc2cc(F)c(Cl)cc12]

In analogy to example 118), 100 mg (0.34 mmol) 1-[(5-cyclopropyl-1,2-oxazol-3-yl)methyl]-3,5-dimethyl-1H-pyrazol-4-amine (intermediate 90C) and 77 mg (0.29 mmol) 2-carbamoyl-6-chloro-7-fluoroquinoline-4-carboxylic acid (intermediate 32A) were reacted to give after a purification via preparative HPLC (method 3) 45 mg (26%) of the desired title compound.

¹H NMR (400 MHz, DMSO d₆): δ (ppm)=0.88 (m, 2H), 1.04 (m, 2H), 2.12 (s, 3H), 2.13 (m, 1H), 2.22 (s, 3H), 5.25 (s, 2H), 6.08 (s, 1H), 7.99 (br. s., 1H), 8.14 (d, 1H), 8.38 (s, 1H), 8.38 (br. s., 1H), 8.51 (d, 1H), 10.21 (s, 1H).

### Example 418

### N⁴-{1-[(5-cyclopropyl-1,2-oxazol-3-yl)methyl]-3,5-dimethyl-1H-pyrazol-4-yl}-7-fluoroquinoline-2,4-dicarboxamide

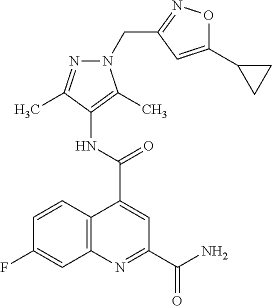 [SMILES: Cc1nn(Cc2cc(C3CC3)on2)c(C)c1NC(=O)c1cc(C(N)=O)nc2cc(F)ccc12]

In analogy to example 118), 100 mg (0.34 mmol) 1-[(5-cyclopropyl-1,2-oxazol-3-yl)methyl]-3,5-dimethyl-1H-pyrazol-4-amine (intermediate 90C) and 67 mg (0.29 mmol) 2-carbamoyl-7-fluoroquinoline-4-carboxylic acid (intermediate 37A) were reacted to give after a purification via preparative HPLC (method 3) 109 mg (67%) of the desired title compound.

¹H NMR (400 MHz, DMSO d₆): δ (ppm)=0.88 (m, 2H), 1.03 (m, 2H), 2.12 (s, 3H), 2.12 (m, 1H), 2.23 (s, 3H), 5.25 (s, 2H), 6.07 (s, 1H), 7.78 (ddd, 1H), 7.92 (dd, 1H), 7.95 (br. s., 1H), 8.28 (s, 1H), 8.38 (br. s., 1H), 8.35 (dd, 1H), 10.11 (s, 1H).

### Example 419

### N⁴-{1-[(5-cyclopropyl-1,2-oxazol-3-yl)methyl]-3,5-dimethyl-1H-pyrazol-4-yl}quinoline-2,4-dicarboxamide

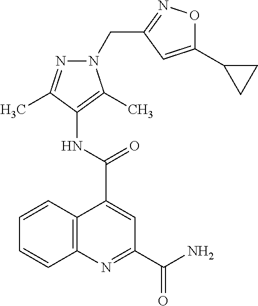 [SMILES: Cc1nn(Cc2cc(C3CC3)on2)c(C)c1NC(=O)c1cc(C(N)=O)nc2ccccc12]

In analogy to example 118), 100 mg (0.34 mmol) 1-[(5-cyclopropyl-1,2-oxazol-3-yl)methyl]-3,5-dimethyl-1H-pyrazol-4-amine (intermediate 90C) and 62 mg (0.29 mmol) 2-carbamoylquinoline-4-carboxylic acid (intermediate 4A) were reacted to give after a purification via preparative HPLC (method 3) 100 mg (64%) of the desired title compound.

¹H NMR (400 MHz, DMSO d₆): δ (ppm)=0.88 (m, 2H), 1.03 (m, 2H), 2.12 (m, 1H), 2.13 (s, 3H), 2.24 (s, 3H), 5.25 (s, 2H), 6.08 (s, 1H), 7.82 (ddd, 1H), 7.90 (br. s., 1H), 7.94 (ddd, 1H), 8.28 (s, 1H), 8.39 (br. s., 1H), 8.21 (dd, 1H), 8.26 (dd, 1H), 10.06 (s, 1H).

### Example 420

### 6-chloro-7-fluoro-N⁴-{5-methyl-1-[(3-methyl-1,2-oxazol-5-yl)methyl]-3-(trifluoromethyl)-1H-pyrazol-4-yl}quinoline-2,4-dicarboxamide

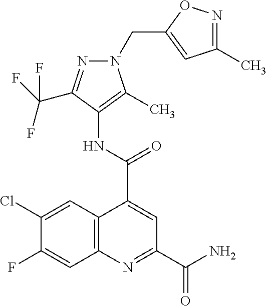 [SMILES: Cc1cc(Cn2nc(C(F)(F)F)c(NC(=O)c3cc(C(N)=O)nc4cc(F)c(Cl)cc34)c2C)on1]

In analogy to example 118), 100 mg (0.35 mmol) 5-methyl-1-[(3-methyl-1,2-oxazol-5-yl)methyl]-3-(trifluoromethyl)-1H-pyrazol-4-amine (intermediate 91C) and 82 mg (0.29 mmol) 2-carbamoyl-6-chloro-7-fluoroquinoline-4-carboxylic acid (intermediate 32A) were reacted to give after a purification via preparative HPLC (method 3) 19 mg (10%) of the desired title compound.

¹H NMR (400 MHz, DMSO d₆): δ (ppm)=2.24 (s, 3H), 2.36 (s, 3H), 5.68 (s, 2H), 6.43 (s, 1H), 8.01 (br. s., 1H), 8.15 (d, 1H), 8.37 (s, 1H), 8.40 (br. s., 1H), 8.43 (d, 1H), 10.55 (s, 1H).

### Example 421

### 6-chloro-N⁴-{1-[(5-cyano-2-thienyl)methyl]-5-methyl-3-(trifluoromethyl)-1H-pyrazol-4-yl}-7-fluoroquinoline-2,4-dicarboxamide

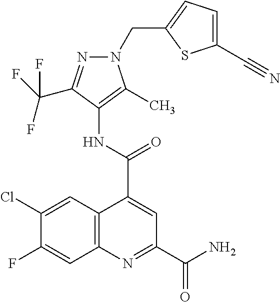 [SMILES: Cc1c(NC(=O)c2cc(C(N)=O)nc3cc(F)c(Cl)cc23)c(C(F)(F)F)nn1Cc1ccc(C#N)s1]

In analogy to example 118), 255 mg (0.58 mmol) 5-{[4-amino-5-methyl-3-(trifluoromethyl)-1H-pyrazol-1-yl]methyl}thiophene-2-carbonitrile (intermediate 92C) and 136 mg (0.48 mmol) 2-carbamoyl-6-chloro-7-fluoroquinoline-4-carboxylic acid (intermediate 32A) were reacted to give after a purification via preparative HPLC (method 3) 38 mg (12%) of the desired title compound.

¹H NMR (400 MHz, DMSO d₆): δ (ppm)=2.35 (s, 3H), 5.80 (s, 2H), 7.31 (d, 1H), 7.91 (d, 1H), 8.00 (br. s., 1H), 8.15 (d, 1H), 8.37 (s, 1H), 8.40 (br. s., 1H), 8.43 (d, 1H), 10.55 (s, 1H).

### Example 422

### N⁴-{1-[(5-cyano-2-thienyl)methyl]-5-methyl-3-(trifluoromethyl)-1H-pyrazol-4-yl}-7-fluoroquinoline-2,4-dicarboxamide

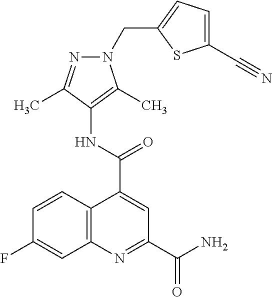 [SMILES: Cc1nn(Cc2ccc(C#N)s2)c(C)c1NC(=O)c1cc(C(N)=O)nc2cc(F)ccc12]

In analogy to example 118), 180 mg (0.50 mmol) 5-[(4-amino-3,5-dimethyl-1H-pyrazol-1-yl)methyl]thiophene-2-carbonitrile (intermediate 93C) and 115 mg (0.42 mmol) 2-carbamoyl-7-fluoroquinoline-4-carboxylic acid (intermediate 37A) were reacted to give after a purification via preparative HPLC (method 3) 26 mg (11%) of the desired title compound.

¹H NMR (400 MHz, DMSO d₆): δ (ppm)=2.15 (s, 3H), 2.25 (s, 3H), 5.55 (s, 2H), 7.23 (d, 1H), 7.88 (d, 1H), 7.77 (ddd, 1H), 7.92 (dd, 1H), 7.95 (br. s., 1H), 8.28 (s, 1H), 8.38 (br. s., 1H), 8.35 (dd, 1H), 10.13 (s, 1H).

### Example 423

### N⁴-{1-[(5-cyanopyrimidin-2-yl)methyl]-5-methyl-3-(trifluoromethyl)-1H-pyrazol-4-yl}-7-fluoroquinoline-2,4-dicarboxamide

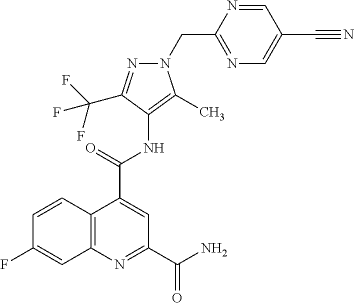 [SMILES: Cc1c(NC(=O)c2cc(C(N)=O)nc3cc(F)ccc23)c(C(F)(F)F)nn1Cc1ncc(C#N)cn1]

In analogy to example 118), 145 mg (0.51 mmol, purity 50%) 2-{[4-amino-5-methyl-3-(trifluoromethyl)-1H-pyrazol-1-yl]methyl}pyrimidine-5-carbonitrile (intermediate 94C) and 100 mg (0.43 mmol) 2-carbamoyl-7-fluoroquinoline-4-carboxylic acid (intermediate 37A) were reacted to give after a purification via preparative HPLC (method 5c) 26 mg (11%) of the desired title compound.

¹H-NMR (400 MHz, DMSO d₆) δ (ppm)=2.29 (s, 3H), 5.87 (s, 2H), 7.82 (td, 1H), 7.94 (dd, 1H), 7.98 (s, 1H), 8.27-8.35 (m, 2H), 8.41 (s, 1H), 9.34 (s, 2H), 10.49 (s, 1H).

### Example 424

### N⁴-{1-[(5-cyanopyrimidin-2-yl)methyl]-5-methyl-3-(trifluoromethyl)-1H-pyrazol-4-yl}-5-fluoroquinoline-2,4-dicarboxamide

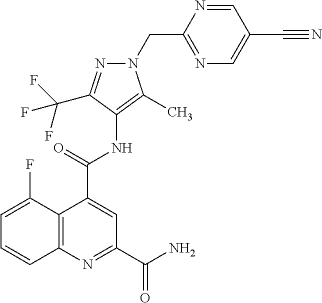 [SMILES: Cc1c(NC(=O)c2cc(C(N)=O)nc3cccc(F)c23)c(C(F)(F)F)nn1Cc1ncc(C#N)cn1]

In analogy to example 118), 400 mg (0.71 mmol, purity 50%) 2-{[4-amino-5-methyl-3-(trifluoromethyl)-1H-pyrazol-1-yl]methyl}pyrimidine-5-carbonitrile (intermediate 94C) and 138 mg (0.59 mmol) 2-carbamoyl-5-fluoroquinoline-4-carboxylic acid (intermediate 39A) were reacted to give after a purification via preparative HPLC (method 5c) 93 mg (31%) of the desired title compound.

¹H-NMR (400 MHz, DMSO d₆) δ (ppm)=2.27 (s, 3H), 5.85 (s, 2H), 7.80 (td, 1H), 7.88-7.99 (m, 2H), 8.25-8.34 (m, 2H), 8.38 (s, 1H), 9.32 (s, 2H), 10.46 (s, 1H).

### Example 425

### N⁴-[1-(4-cyanobenzyl)-5-methyl-3-(trifluoromethyl)-1H-pyrazol-4-yl]-6-hydroxyquinoline-2,4-dicarboxamide

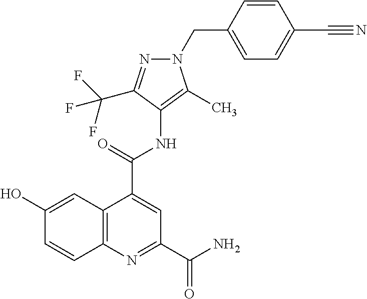 [SMILES: Cc1c(NC(=O)c2cc(C(N)=O)nc3ccc(O)cc23)c(C(F)(F)F)nn1Cc1ccc(C#N)cc1]

In analogy to example 164), 250 mg (0.49 mmol) N4-[1-(4-cyanobenzyl)-5-methyl-3-(trifluoromethyl)-1H-pyrazol-4-yl]-6-methoxyquinoline-2,4-dicarboxamide (example 245) were reacted to give after a purification of the raw material via a Biotage chromatography system (25 g snap KP-Sil column, ethyl acetate/40-100% methanol) and followed by a preparative HPLC (method 5c) 32 mg (12%) of the desired title compound.

¹H-NMR (400 MHz, DMSO d₆) δ (ppm)=2.27 (s, 3H), 5.62 (s, 2H), 7.39 (d, 2H), 7.44-7.51 (m, 2H), 7.78 (d, 1H), 7.88-7.95 (m, 2H), 8.04-8.08 (m, 1H), 8.17 (s, 1H), 8.26 (d, 1H), 10.34 (s, 1H), 10.55 (s, 1H).

### Example 426

### N⁴-[1-(4-cyanobenzyl)-5-methyl-3-(trifluoromethyl)-1H-pyrazol-4-yl]-N²-(methylsulfonyl)quinoline-2,4-dicarboxamide

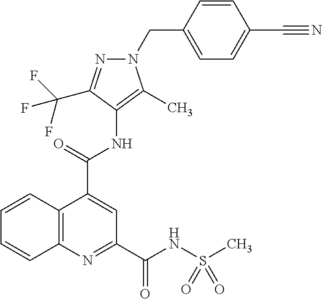 [SMILES: Cc1c(NC(=O)c2cc(C(=O)NS(C)(=O)=O)nc3ccccc23)c(C(F)(F)F)nn1Cc1ccc(C#N)cc1]

In analogy to example 118), 137 mg (0.492 mmol) 4-{[4-amino-5-methyl-3-(trifluoromethyl)-1H-pyrazol-1-yl]methyl}benzonitrile (intermediate 26C) and 120 mg (0.41 mmol) 2-[(methylsulfonyl)carbamoyl]quinoline-4-carboxylic acid (intermediate 60A) were reacted to give after a purification via preparative HPLC (method 5d) 76 mg (30%) of the desired title compound.

¹H-NMR (400 MHz, DMSO d₆) δ (ppm)=2.29 (s, 3H), 3.45 (s, 3H), 5.62 (s, 2H), 7.4 (d, 2H), 7.87-793 (m, 3H), 8.01 (dt, 1H), 8.22-8.27 (m, 2H), 8.33 (d, 1H), 10.47 (s, 1H), 11.76 (s, 1H).

### Example 427

### N⁴-{1-[(6-cyanopyridazin-3-yl)methyl]-5-methyl-3-(trifluoromethyl)-1H-pyrazol-4-yl}-7-fluoroquinoline-2,4-dicarboxamide

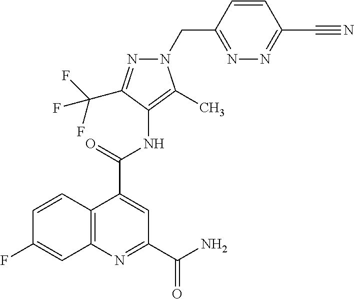 [SMILES: Cc1c(NC(=O)c2cc(C(N)=O)nc3cc(F)ccc23)c(C(F)(F)F)nn1Cc1ccc(C#N)nn1]

In analogy to example 118), 80 mg (0.11 mmol, purity about 40%) 6-{[4-amino-5-methyl-3-(trifluoromethyl)-1H-pyrazol-1-yl]methyl}pyridazine-3-carbonitrile (intermediate 95C) and 22 mg (0.094 mmol) 2-carbamoyl-7-fluoroquinoline-4-carboxylic acid (intermediate 37A) were reacted to give after a purification via preparative HPLC (method 5c) 12 mg (25%) of the desired title compound.

¹H-NMR (400 MHz, DMSO d₆) δ (ppm)=2.37 (s, 3H), 5.96 (s, 2H), 7.82 (td, 1H), 7.90-7.96 (m, 2H), 8.00 (d, 1H), 8.27-8.33 (m, 2H), 8.41-8.46 (m, 2H), 10.53 (s, 1H).

### Additional Examples

In addition to the examples described with explicit synthesis protocols supra, we have produced further compounds Listed in the following table using compounds and synthetic methods either described in the protocols and the general synthesis description, supra, or which are well known to the person skilled in the art.

Lengthy table referenced here
US20170037028A1-20170209-T00001
Please refer to the end of the specification for access instructions.

Further, the compounds of formula (I) of the present invention can be converted to any salt as described herein, by any method which is known to the person skilled in the art. Similarly, any salt of a compound of formula (I) of the present invention can be converted into the free compound, by any method which is known to the person skilled in the art.

### Biological In Vitro Assays

The example testing experiments described herein serve to illustrate the present invention and the invention is not limited to the examples given.

### Biological Evaluation

In order that this invention may be better understood, the following examples are set forth. These examples are for the purpose of illustration only, and are not to be construed as limiting the scope of the invention in any manner. All publications mentioned herein are incorporated by reference in their entirety. Demonstration of the activity of the compounds of the present invention may be accomplished through in vitro and in vivo assays that are well known in the art. For example, to demonstrate the efficacy of a pharmaceutical agent to inhibit glucose transporter GLUT1 and/or GLUT2 the following assays may be used.

### Indirect Measurement of GLUT1 and GLUT2 Activity by Quantification of Intracellular ATP Levels

It is well known that a combination of small-molecule inhibitors of mitochondrial electron transport chain and glucose catabolism synergistically suppress ATP production and impair cellular viability (Ulanovskaya et al., 2008, 2011; Liu, et al. 2001). We therefore used DLD1 or CHO-K1 cells in combination with an oxidative phosphorylation inhibitor to identify GLUT1 and GLUT2 inhibitors. Cell lines were maintained in DMEM medium supplemented with 10% FCS and 1% Penicillin-Streptomycin solution and 2% Glutamax. The cells were treated with trypsin and seeded into 384 plates at a density of 4000 cells/well. The cells were then cultured overnight in glucose free media containing 1% FCS to reduce intracellular ATP levels. After 24 h the cells were incubated at 37° C. containing the respective appropriate concentrations of glucose (0.1 mM, for determining GLUT1 activity) or fructose (10 mM, for determining GLUT2 activity) with or without example compounds and 1 μM Rotenone for 15 min. The CellTiter-Glo® Luminescent Cell Viability Assay from Promega was then used to measure ATP Levels. Compounds able to reduce the ATP levels within 15 min of glucose/fructose application were considered to be glucose/fructose uptake inhibitors, respectively.

TABLE 1
Measured IC₅₀ values of compounds regarding glucose induced ATP
increase (GLUT1 inhibition)

| Example | IC₅₀ [nM]¹ |
|---|---|
| 1 | 1.6 |
| 2 | 2.8 |
| 3 | 3.1 |
| 4 | 3.8 |
| 5 | 4.0 |
| 6 | 4.7 |
| 7 | 4.9 |
| 8 | 5.4 |
| 9 | 6.2 |
| 10 | 6.4 |
| 11 | 6.6 |
| 12 | 7.7 |
| 13 | 11.3 |
| 14 | 11.5 |
| 15 | 11.6 |
| 16 | 12.1 |
| 17 | 12.2 |
| 18 | 12.4 |
| 19 | 12.7 |
| 20 | 12.7 |
| 21 | 14.8 |
| 22 | 15.4 |
| 23 | 15.4 |
| 24 | 15.5 |
| 25 | 15.7 |
| 26 | 15.7 |
| 27 | 16.5 |
| 28 | 16.8 |
| 29 | 19.0 |
| 30 | 19.5 |
| 31 | 20.0 |
| 32 | 20.5 |
| 33 | 25.0 |
| 34 | 25.2 |
| 35 | 25.6 |
| 36 | 88.9 |
| 37 | 26.7 |
| 38 | 29.5 |
| 39 | 30.0 |
| 40 | 30.0 |
| 41 | 30.0 |
| 42 | 30.9 |
| 43 | 31.7 |
| 44 | 32.1 |
| 45 | 35.4 |
| 46 | 40.0 |
| 47 | 44.4 |
| 48 | 44.4 |
| 49 | 50.0 |
| 50 | 50.0 |
| 51 | 50.0 |
| 52 | 52.1 |
| 53 | 52.4 |
| 54 | 58.3 |
| 55 | 59.3 |
| 56 | 60.4 |
| 57 | 66.7 |
| 58 | 77.1 |
| 59 | 92.6 |
| 60 | 97.9 |
| 61 | 110.0 |
| 62 | 127.0 |
| 63 | 129.0 |
| 64 | 130.0 |
| 65 | 133.0 |
| 66 | 135.0 |
| 67 | 146.0 |
| 68 | 148.0 |
| 69 | 164.0 |
| 70 | 173.0 |
| 71 | 175.0 |
| 72 | 177.0 |
| 73 | 177.0 |
| 74 | 181.0 |
| 75 | 182.0 |
| 76 | 188.0 |
| 77 | 204.0 |
| 78 | 223.0 |
| 79 | 225.0 |
| 80 | 229.0 |
| 81 | 229.0 |
| 82 | 250.0 |
| 83 | 250.0 |
| 84 | 255.0 |
| 85 | 257.0 |
| 86 | 257.0 |
| 87 | 267.0 |
| 88 | 281.0 |
| 89 | 281.0 |
| 90 | 291.0 |
| 91 | 292.0 |
| 92 | 292.0 |
| 93 | 292.0 |
| 94 | 304.0 |
| 95 | 309.0 |
| 96 | 344.0 |
| 97 | 354.0 |
| 98 | 362.0 |
| 99 | 364.0 |
| 100 | 371.0 |
| 101 | 396.0 |
| 102 | 446.0 |
| 103 | 462.0 |
| 104 | 519.0 |
| 105 | 528.0 |
| 106 | 571.0 |
| 107 | 593.0 |
| 108 | 671.0 |
| 109 | 688.0 |
| 110 | 708.0 |
| 111 | 708.0 |
| 112 | 771.0 |
| 113 | 813.0 |
| 114 | 833.0 |
| 115 | 833.0 |
| 116 | 889.0 |
| 117 | 938.0 |
| 118 | 0.5 |
| 119 | 1.3 |
| 120 | 1.5 |
| 121 | 1.7 |
| 122 | 1.9 |
| 123 | 2.0 |
| 124 | 2.3 |
| 125 | 2.5 |
| 126 | 4.7 |
| 127 | 3.1 |
| 128 | 3.3 |
| 129 | 4.1 |
| 130 | 4.6 |
| 131 | 8.3 |
| 132 | 5560.0 |
| 133 | 5.6 |
| 134 | 6.6 |
| 135 | 8.1 |
| 136 | 9.0 |
| 137 | 10.7 |
| 138 | 18.1 |
| 139 | 31.7 |
| 140 | 50.0 |
| 141 | 69.4 |
| 142 | 70.7 |
| 143 | 85.3 |
| 144 | 5.7 |
| 145 | 6.1 |
| 146 | 7.9 |
| 147 | 8.2 |
| 148 | 11.4 |
| 149 | 11.7 |
| 150 | 11.7 |
| 151 | 1280.0 |
| 152 | 58.3 |
| 153 | 95.8 |
| 154 | 196.0 |
| 155 | 312.0 |
| 156 | 354.0 |
| 157 | 437.0 |
| 158 | 521.0 |
| 159 | 875.0 |
| 160 | 14.3 |
| 161 | 15.1 |
| 162 | 24.8 |
| 163 | 25.0 |
| 164 | 27.1 |
| 165 | 30.4 |
| 166 | 41.1 |
| 167 | 46.2 |
| 168 | 47.8 |
| 169 | 50.5 |
| 170 | 54.4 |
| 171 | 57.1 |
| 172 | 59.1 |
| 173 | 65.2 |
| 174 | 69.6 |
| 175 | 70.7 |
| 176 | 72.2 |
| 177 | 73.9 |
| 178 | 78.5 |
| 179 | 78.5 |
| 180 | 93.7 |
| 181 | 93.8 |
| 182 | 101.0 |
| 183 | 107.0 |
| 184 | 110.0 |
| 185 | 122.0 |
| 186 | 126.0 |
| 187 | 131.0 |
| 188 | 161.0 |
| 189 | 165.0 |
| 190 | 171.0 |
| 191 | 171.0 |
| 192 | 177.0 |
| 193 | 215.0 |
| 194 | 229.0 |
| 195 | 272.0 |
| 196 | 286.0 |
| 197 | 333.0 |
| 198 | 438.0 |
| 199 | 441.0 |
| 200 | 539.0 |
| 201 | 557.0 |
| 202 | 562.0 |
| 203 | 602.0 |
| 204 | 613.0 |
| 205 | 613.0 |
| 206 | 667.0 |
| 207 | 688.0 |
| 208 | 741.0 |
| 209 | 817.0 |
| 210 | 849.0 |
| 211 | 957.0 |
| 212 | 0.6 |
| 213 | 0.7 |
| 214 | 1.1 |
| 215 | 1.6 |
| 216 | 2.1 |
| 217 | 3.2 |
| 218 | 3.4 |
| 219 | 5.4 |
| 220 | 6.4 |
| 221 | 6.7 |
| 222 | 7.1 |
| 223 | 11.1 |
| 224 | 11.2 |
| 225 | 14.0 |
| 226 | 4.0 |
| 227 | 4.6 |
| 228 | 4.6 |
| 229 | 4.6 |
| 230 | 5.0 |
| 231 | 5.8 |
| 232 | 7.0 |
| 233 | 7.2 |
| 234 | 9.2 |
| 235 | 10.9 |
| 235 | 10.9 |
| 236 | 0.4 |
| 237 | 0.4 |
| 238 | 1.1 |
| 239 | 2.9 |
| 240 | 2.2 |
| 241 | 1.1 |
| 242 | 1.0 |
| 243 | 0.5 |
| 244 | 17.6 |
| 245 | 0.3 |
| 246 | 2.6 |
| 247 | 4.9 |
| 248 | 0.6 |
| 249 | 1.0 |
| 250 | 3.6 |
| 251 | 0.9 |
| 252 | 1.2 |
| 253 | 0.5 |
| 254 | 1.2 |
| 255 | 1.4 |
| 256 | 2.4 |
| 257 | 2.4 |
| 258 | 1.0 |
| 259 | 9.7 |
| 260 | 4.7 |
| 261 | 0.5 |
| 262 | 1.2 |
| 263 | 3.9 |
| 264 | 2.6 |
| 265 | 6.6 |
| 266 | 1.0 |
| 267 | 2.6 |
| 268 | 0.6 |
| 269 | 1.5 |
| 270 | 1.8 |
| 271 | 3.6 |
| 272 | 2.5 |
| 273 | 5.9 |
| 274 | 1.0 |
| 275 | 1.1 |
| 276 | 1.3 |
| 277 | 1.8 |
| 278 | 1.4 |
| 279 | 1.4 |
| 280 | 1.1 |
| 281 | 1.3 |
| 282 | 3.2 |
| 283 | 1.2 |
| 284 | 9.0 |
| 285 | 3.4 |
| 286 | 35.6 |
| 287 | 4.8 |
| 288 | 40.1 |
| 289 | 21.5 |
| 290 | 31.7 |
| 291 | 6.9 |
| 292 | 17.0 |
| 293 | 1.0 |
| 294 | 6.6 |
| 295 | 1020.0 |
| 296 | 0.6 |
| 297 | 0.8 |
| 298 | 1.0 |
| 299 | 6.8 |
| 300 | 0.5 |
| 301 | 0.5 |
| 302 | 1.1 |
| 303 | 1.8 |
| 304 | 3.6 |
| 305 | 1.4 |
| 306 | 4.7 |
| 307 | 5.1 |
| 308 | 2.7 |
| 309 | 363.0 |
| 310 | 47.2 |
| 311 | 6.0 |
| 312 | 35.5 |
| 313 | 185.0 |
| 314 | 1.6 |
| 315 | 16.0 |
| 316 | 19.8 |
| 317 | 1050.0 |
| 318 | 352.0 |
| 319 | 1.0 |
| 320 | 8.0 |
| 321 | 7.2 |
| 322 | 1.9 |
| 323 | 39.4 |
| 324 | 1.9 |
| 325 | 3.9 |
| 326 | 14.3 |
| 327 | 3.7 |
| 329 | 1.1 |
| 330 | 1.3 |
| 331 | 2.5 |
| 332 | 3.5 |
| 333 | 0.6 |
| 334 | 7.5 |
| 335 | 1.6 |
| 336 | 150.0 |
| 337 | 1.9 |
| 338 | 2.7 |
| 339 | 23.9 |
| 340 | 81.8 |
| 341 | 290.0 |
| 342 | 336.0 |
| 343 | 0.7 |
| 344 | 5.8 |
| 345 | 7.2 |
| 346 | 0.6 |
| 347 | 2.2 |
| 348 | 6.1 |
| 349 | 15.7 |
| 350 | 1.1 |
| 351 | 1.6 |
| 352 | 2.5 |
| 353 | 2.3 |
| 354 | 3.6 |
| 355 | 3.9 |
| 356 | 9.3 |
| 357 | 28.8 |
| 358 | 1.2 |
| 359 | 1.3 |
| 360 | 2.0 |
| 361 | 4.4 |
| 362 | 5.5 |
| 363 | 6.0 |
| 364 | 6.6 |
| 365 | 26.6 |
| 366 | 7.0 |
| 367 | 2.3 |
| 368 | 418.0 |
| 369 | 12.4 |
| 370 | 12.8 |
| 371 | 243.0 |
| 372 | 20.0 |
| 373 | 17.5 |
| 374 | 895.0 |
| 375 | 123.0 |
| 376 | 642.0 |
| 377 | 1100.0 |
| 378 | 665.0 |
| 379 | 862.0 |
| 380 | 45.9 |
| 381 | 9.0 |
| 382 | 26.1 |
| 383 | 8.3 |
| 384 | 3.6 |
| 385 | 81.7 |
| 386 | 43.2 |
| 387 | 146.0 |
| 388 | 108.0 |
| 389 | 1.9 |
| 390 | 5.5 |
| 391 | 7.0 |
| 392 | 21.6 |
| 393 | 4.1 |
| 394 | 2.6 |
| 395 | 5.3 |
| 396 | 15.9 |
| 397 | 7.5 |
| 398 | 5.5 |
| 399 | 4.5 |
| 400 | 1260.0 |
| 401 | 10.0 |
| 402 | 6.7 |
| 403 | 1.0 |
| 404 | 1.4 |
| 405 | 242.0 |
| 406 | 1530.0 |
| 407 | 2.1 |
| 408 | 7.8 |
| 409 | 5.5 |
| 410 | 8.9 |
| 411 | 1.1 |
| 412 | 2.1 |
| 413 | 5.6 |
| 414 | 1.3 |
| 415 | 10.0 |
| 416 | 2.7 |
| 417 | 0.7 |
| 418 | 4.1 |
| 419 | 4.2 |
| 420 | 4.9 |
| 421 | 2.9 |
| 422 | 5.0 |
| 423 | 6.7 |
| 424 | 6.7 |
| 425 | 4.9 |
| 426 | 2530.0 |
| 427 | 78.3 |
| 1018 | 1.9 |
| 1019 | 1.9 |
| 1020 | 1.9 |
| 1021 | 2.3 |
| 1022 | 2.4 |
| 1024 | 2.7 |
| 1025 | 2.8 |
| 1027 | 3.0 |
| 1028 | 3.0 |
| 1029 | 3.0 |
| 1030 | 3.2 |
| 1033 | 4.2 |
| 1035 | 5.0 |
| 1036 | 5.1 |
| 1037 | 5.3 |
| 1038 | 5.7 |
| 1039 | 5.7 |
| 1040 | 6.7 |
| 1041 | 8.6 |
| 1042 | 8.7 |
| 1043 | 8.8 |
| 1044 | 8.8 |
| 1045 | 9.4 |
| 1046 | 9.6 |
| 1047 | 11.2 |
| 1048 | 11.5 |
| 1049 | 11.7 |
| 1051 | 11.8 |
| 1052 | 13.1 |
| 1053 | 13.7 |
| 1054 | 13.9 |
| 1055 | 14.4 |
| 1056 | 15.0 |
| 1057 | 15.1 |
| 1058 | 15.3 |
| 1059 | 15.6 |
| 1060 | 15.8 |
| 1061 | 15.8 |
| 1062 | 16.4 |
| 1063 | 16.8 |
| 1064 | 18.6 |
| 1065 | 18.7 |
| 1066 | 19.1 |
| 1067 | 19.2 |
| 1068 | 19.5 |
| 1069 | 19.8 |
| 1070 | 20.2 |
| 1071 | 21.0 |
| 1072 | 21.0 |
| 1073 | 21.7 |
| 1074 | 22.3 |
| 1075 | 22.5 |
| 1076 | 22.7 |
| 1077 | 23.1 |
| 1078 | 23.9 |
| 1079 | 24.4 |
| 1080 | 25.8 |
| 1081 | 26.3 |
| 1082 | 28.2 |
| 1083 | 29.4 |
| 1084 | 30.8 |
| 1085 | 31.2 |
| 1086 | 31.6 |
| 1087 | 32.2 |
| 1088 | 34.9 |
| 1089 | 35.5 |
| 1091 | 36.0 |
| 1092 | 37.9 |
| 1093 | 38.4 |
| 1094 | 39.3 |
| 1096 | 41.1 |
| 1097 | 41.3 |
| 1098 | 41.4 |
| 1099 | 41.9 |
| 1100 | 42.1 |
| 1101 | 42.8 |
| 1102 | 43.5 |
| 1103 | 44.7 |
| 1104 | 47.8 |
| 1105 | 48.2 |
| 1106 | 49.6 |
| 1107 | 52.2 |
| 1108 | 52.5 |
| 1109 | 54.2 |
| 1110 | 55.4 |
| 1111 | 56.8 |
| 1112 | 57.0 |
| 1113 | 58.5 |
| 1114 | 59.2 |
| 1115 | 59.4 |
| 1116 | 59.5 |
| 1117 | 59.7 |
| 1118 | 63.5 |
| 1119 | 65.1 |
| 1120 | 65.5 |
| 1121 | 65.5 |
| 1122 | 67.3 |
| 1123 | 71.1 |
| 1124 | 72.6 |
| 1125 | 78.5 |
| 1126 | 80.0 |
| 1127 | 83.3 |
| 1128 | 86.5 |
| 1129 | 86.6 |
| 1130 | 86.6 |
| 1131 | 86.7 |
| 1132 | 88.2 |
| 1133 | 90.1 |
| 1134 | 97.3 |
| 1135 | 98.1 |
| 1136 | 98.2 |
| 1137 | 100.1 |
| 1138 | 101.8 |
| 1139 | 104.7 |
| 1140 | 105.1 |
| 1141 | 108.0 |
| 1142 | 111.9 |
| 1143 | 113.8 |
| 1144 | 114.0 |
| 1145 | 116.2 |
| 1146 | 116.6 |
| 1147 | 117.5 |
| 1148 | 122.3 |
| 1149 | 126.5 |
| 1150 | 127.9 |
| 1151 | 128.4 |
| 1152 | 132.4 |
| 1153 | 135.8 |
| 1154 | 138.9 |
| 1155 | 139.7 |
| 1156 | 149.4 |
| 1157 | 149.6 |
| 1158 | 154.7 |
| 1159 | 160.0 |
| 1160 | 161.2 |
| 1161 | 161.7 |
| 1162 | 164.4 |
| 1163 | 165.6 |
| 1164 | 167.0 |
| 1165 | 168.2 |
| 1166 | 170.5 |
| 1167 | 171.3 |
| 1168 | 179.3 |
| 1169 | 182.2 |
| 1170 | 189.4 |
| 1171 | 194.6 |
| 1172 | 197.6 |
| 1173 | 198.4 |
| 1174 | 203.2 |
| 1175 | 205.0 |
| 1176 | 207.9 |
| 1177 | 220.6 |
| 1178 | 224.1 |
| 1179 | 228.3 |
| 1180 | 230.7 |
| 1181 | 237.0 |
| 1182 | 238.3 |
| 1183 | 241.4 |
| 1184 | 242.1 |
| 1185 | 246.5 |
| 1186 | 277.1 |
| 1187 | 288.4 |
| 1188 | 289.8 |
| 1189 | 310.4 |
| 1190 | 311.8 |
| 1191 | 335.4 |
| 1192 | 339.4 |
| 1193 | 342.7 |
| 1194 | 363.3 |
| 1195 | 368.1 |
| 1196 | 375.3 |
| 1197 | 385.0 |
| 1198 | 389.9 |
| 1199 | 394.4 |
| 1200 | 407.8 |
| 1201 | 427.8 |
| 1202 | 430.4 |
| 1203 | 432.8 |
| 1204 | 436.3 |
| 1205 | 450.0 |
| 1206 | 451.4 |
| 1207 | 476.5 |
| 1208 | 480.8 |
| 1209 | 501.5 |
| 1210 | 572.3 |
| 1211 | 580.8 |
| 1212 | 598.2 |
| 1213 | 614.3 |
| 1214 | 622.1 |
| 1215 | 648.7 |
| 1216 | 672.7 |
| 1217 | 675.5 |
| 1218 | 694.5 |
| 1219 | 708.5 |
| 1220 | 723.0 |
| 1221 | 748.8 |
| 1222 | 883.2 |
| 1223 | 891.5 |
| 1224 | 912.3 |
| 1225 | 914.0 |
| 1226 | 923.7 |
| 1227 | 961.3 |
| 1228 | 961.6 |
| 1229 | 999.4 |
| 1230 | 1000.0 |
| 1231 | 1043.8 |
| 1232 | 1045.5 |
| 1233 | 1113.4 |
| 1234 | 1175.1 |
| 1235 | 1282.1 |
| 1236 | 1291.7 |
| 1237 | 1318.2 |
| 1238 | 1370.4 |
| 1239 | 1473.7 |
| 1240 | 1500.0 |
| 1241 | 1528.1 |
| 1242 | 1562.4 |
| 1243 | 1592.6 |
| 1244 | 1592.6 |
| 1245 | 1604.5 |
| 1246 | 1657.1 |
| 1247 | 1666.6 |
| 1248 | 1666.7 |
| 1249 | 1666.7 |
| 1250 | 1671.3 |
| 1251 | 1868.1 |
| 1252 | 1869.6 |
| 1253 | 1971.4 |
| 1254 | 1979.2 |
| 1255 | 2009.2 |
| 1256 | 2037.0 |
| 1257 | 2053.4 |
| 1258 | 2061.4 |
| 1259 | 2100.7 |
| 1260 | 2166.7 |
| 1261 | 2291.7 |
| 1262 | 2292.0 |
| 1263 | 2315.8 |
| 1264 | 2378.8 |
| 1265 | 2484.1 |
| 1266 | 2491.7 |
| 1267 | 2708.3 |
| 1268 | 2846.2 |
| 1269 | 2888.9 |
| 1270 | 2890.8 |
| 1271 | 3057.1 |
| 1272 | 3181.8 |
| 1273 | 3372.3 |
| 1274 | 3405.7 |
| 1275 | 3496.2 |
| 1276 | 3809.5 |
| 1277 | 3846.2 |
| 1278 | 3909.1 |
| 1279 | 3932.0 |
| 1280 | 3934.9 |
| 1281 | 5185.2 |
| 1282 | 6023.6 |
| 1283 | 6250.0 |
| 1284 | 6250.0 |
| 1285 | 6250.0 |
| 1286 | 6250.0 |
| 1287 | 6250.0 |
| 1288 | 6250.0 |
| 1289 | 6250.0 |
| 1290 | 7105.8 |
| 1291 | 7194.5 |
| 1292 | 7384.5 |
| 1293 | 7790.7 |
| 1294 | 8125.0 |
| 1295 | 8148.1 |
| 1296 | 11111.1 |
| 1297 | 11111.1 |
| 1298 | 11111.1 |
| 1299 | 11111.1 |
| 1300 | 11111.1 |
| 1301 | 11111.1 |
| 1302 | 11111.1 |
| 1303 | 11111.1 |
| 1304 | 11111.1 |
| 1305 | 11111.1 |
| 1306 | 11111.1 |
| 1307 | 14285.7 |
| 1308 | 16923.1 |
| 1309 | 17532.5 |
| 1310 | 27272.7 |
| 1338 | 26.3 |
| 1339 | 72.2 |
| 1341 | 297.0 |
| 1342 | 26.5 |
| 1344 | 323.0 |
| 1346 | 10.6 |
| 1347 | 3950.0 |
| 1348 | 188.0 |
| 1349 | 7.5 |
| 1350 | 41.6 |
| 1351 | 134.0 |
| 1352 | 35.1 |
| 1353 | 38.9 |
| 1354 | 48.9 |
| 1355 | >13043.5 |
| 1356 | >13043.5 |
| 1357 | >13043.5 |
| 1358 | 4.3 |
| 1359 | 22.0 |
| 1360 | 21.6 |
| 1361 | 36.9 |
| 1362 | 5.8 |
| 1363 | 1.8 |
| 1364 | 125.0 |
| 1365 | 167.0 |
| 1366 | 38.1 |
| 1367 | 275.0 |
| 1368 | 29.3 |
| 1369 | 143.0 |
| 1370 | 1090.0 |
| 1371 | 144.0 |
| 1372 | 950.0 |
| 1373 | 1990.0 |
| 1374 | 42.6 |
| 1375 | 15.1 |
| 1376 | 18.9 |
| 1377 | 1.7 |
| 1378 | 643.0 |
| 1379 | 39.4 |
| 1380 | 3.7 |
| 1381 | 1180.0 |
| 1382 | 5370.0 |
| 1383 | 140.0 |
| 1384 | 425.0 |
| 1385 | 60.7 |
| 1386 | 13.6 |
| 1387 | 36.7 |
| 1388 | 1530.0 |
| 1389 | 1930.0 |
| 1390 | 229.0 |
| 1391 | 362.0 |
| 1392 | 15.5 |
| 1393 | 15.0 |
| 1394 | 127.0 |
| 1395 | 12.6 |
| 1396 | 4.6 |
| 1397 | 4.3 |
| 1398 | 38.9 |
| 1399 | 132.0 |
| 1400 | 2610.0 |
| 1401 | 1240.0 |
| 1402 | 238.0 |
| 1403 | 245.0 |
| 1404 | 3.8 |
| 1405 | 8.2 |
| 1406 | 119.0 |
| 1407 | 28.7 |
| 1408 | 4.9 |
| 1409 | 11.1 |
| 1410 | 30.6 |
| 1411 | 3.2 |
| 1412 | 0.9 |
| 1413 | 1.1 |
| 1414 | 0.8 |
| 1415 | 3.3 |
| 1416 | 97.0 |
| 1417 | 164.0 |
| 1418 | 34.1 |
| 1419 | 24.6 |
| 1420 | 354.0 |
| 1421 | 39.0 |
| 1422 | 7110.0 |
| 1423 | 29.6 |
| 1424 | 69.1 |
| 1425 | 56.3 |
| 1426 | 80.0 |
| 1427 | 26.9 |
| 1428 | 12.3 |
| 1429 | 29.4 |
| 1430 | 66.3 |
| 1431 | 138.0 |
| 1432 | 27.4 |
| 1433 | 717.0 |
| 1434 | 51.3 |
| 1435 | 2.8 |
| 1436 | 63.2 |
| 1437 | 13.9 |
| 1438 | 3.1 |
| 1439 | 1.9 |
| 1440 | 5.4 |
| 1441 | 6.5 |
| 1442 | 2.0 |
| 1443 | 4.0 |
| 1444 | 11.6 |
| 1445 | 5.7 |
| 1446 | 1.9 |
| 1447 | 23.2 |
| 1448 | 6.9 |
| 1449 | 18.7 |
| 1450 | 2.3 |
| 1451 | 8.5 |
| 1452 | 8.8 |
| 1453 | 1.4 |
| 1454 | 13.3 |
| 1455 | 20.8 |
| 1456 | 1.5 |
| 1457 | 15.2 |
| 1458 | 2.3 |
| 1459 | 9.8 |
| 1460 | 1.9 |
| 1461 | 8.7 |
| 1462 | 2.9 |
| 1463 | 4.1 |
| 1464 | 5.5 |
| 1465 | 5.1 |
| 1466 | 5.4 |
| 1467 | 7.6 |
| 1468 | 12.9 |
| 1469 | 5.5 |
| 1470 | 2.8 |
| 1471 | 12.3 |
| 1472 | 0.9 |
| 1473 | 5.2 |
| 1474 | 2.1 |
| 1475 | 8.4 |
| 1476 | 2.9 |
| 1477 | 4.3 |
| 1478 | 6.2 |
| 1479 | 22.7 |
| 1480 | 10.5 |
| 1481 | 5.5 |
| 1482 | 5.3 |
| 1483 | 48.8 |
| 1484 | 23.4 |
| 1485 | 5.3 |
| 1486 | 9.6 |
| 1487 | 1.2 |
| 1488 | 6.8 |
| 1489 | 6.0 |
| 1490 | 3.7 |
| 1491 | 41.5 |
| 1492 | 113.0 |
| 1493 | 69.6 |
| 1494 | 8.3 |
| 1495 | 7.3 |
| 1496 | 6.2 |
| 1497 | 4.3 |
| 1498 | 8.0 |
| 1499 | 13.4 |
| 1500 | 20.1 |
| 1501 | 73.7 |
| 1502 | 213.0 |
| 1503 | 93.2 |
| 1504 | 42.4 |
| 1505 | 323.0 |
| 1506 | 35.1 |
| 1507 | 711.0 |
| 1508 | 107.0 |
| 1509 | 16.3 |
| 1510 | 20.0 |
| 1511 | 31.8 |
| 1512 | 105.0 |
| 1513 | 60.8 |
| 1514 | 493.0 |
| 1515 | 319.0 |
| 1516 | 26.1 |
| 1517 | 98.7 |
| 1518 | 69.2 |
| 1519 | 644.0 |
| 1520 | 747.0 |
| 1521 | 1670.0 |
| 1522 | 600.0 |
| 1523 | 4140.0 |
| 1524 | 2020.0 |
| 1525 | 4810.0 |
| 1526 | 556.0 |
| 1527 | 228.0 |
| 1528 | 59.4 |
| 1529 | 402.0 |
| 1530 | 893.0 |
| 1531 | 37.6 |
| 1532 | 16.1 |
| 1533 | 168.0 |
| 1534 | 19.7 |
| 1535 | 22.5 |
| 1536 | 79.1 |
| 1537 | 185.0 |
| 1538 | 73.1 |
| 1539 | 47.3 |
| 1540 | 275.0 |
| 1541 | 15.4 |
| 1542 | 84.9 |
| 1543 | 112.0 |
| 1544 | 15.7 |
| 1545 | 43.1 |
| 1546 | 33.7 |
| 1547 | 24.0 |
| 1548 | 26.5 |
| 1549 | 13.6 |
| 1550 | 34.8 |
| 1551 | 11.1 |
| 1552 | 194.0 |
| 1553 | 159.0 |
| 1554 | 116.0 |
| 1555 | 18.4 |
| 1556 | 41.7 |
| 1557 | 53.8 |
| 1558 | 11.9 |
| 1559 | 72.7 |
| 1560 | 39.8 |
| 1561 | 10.4 |
| 1562 | 55.9 |
| 1563 | 35.5 |
| 1564 | 112.0 |
| 1565 | 21.0 |
| 1566 | 14.2 |
| 1567 | 84.4 |
| 1568 | 27.3 |
| 1569 | 67.5 |
| 1570 | 48.2 |
| 1571 | 64.1 |
| 1572 | 219.0 |
| 1573 | 419.0 |
| 1574 | 49.3 |
| 1575 | 37.9 |
| 1576 | 20.6 |
| 1577 | 9.7 |
| 1578 | 25.0 |
| 1579 | 23.4 |
| 1580 | 1.8 |
| 1581 | 26.9 |
| 1582 | 3.9 |
| 1583 | 11.4 |
| 1584 | 544.0 |
| 1585 | 45.5 |
| 1586 | 36.9 |
| 1587 | 41.8 |
| 1588 | 175.0 |
| 1589 | 13.9 |
| 1590 | 9.8 |
| 1591 | 16.6 |
| 1592 | 1.3 |
| 1593 | 1.3 |
| 1594 | 1.1 |
| 1595 | 1.6 |
| 1596 | 67.2 |
| 1597 | 89.9 |
| 1598 | 1.4 |
| 1599 | 6.5 |
| 1600 | 3.5 |
| 1601 | 24.6 |
| 1602 | 29.3 |
| 1603 | 5.8 |
| 1604 | 4.7 |
| 1605 | 2.6 |
| 1606 | 2.4 |
| 1607 | 3.5 |
| 1608 | 4.0 |
| 1609 | 1.2 |
| 1610 | 8.4 |
| 1611 | 0.8 |
| 1612 | 2.4 |
| 1613 | 0.7 |
| 1614 | 0.8 |
| 1615 | 59.4 |
| 1616 | 67.4 |
| 1617 | 54.9 |
| 1618 | 22.3 |
| 1619 | 40.1 |
| 1620 | 96.3 |
| 1621 | 15.3 |
| 1622 | 50.1 |
| 1623 | 0.8 |
| 1624 | 18.7 |
| 1625 | 40.9 |
| 1626 | 107.0 |
| 1627 | 30.5 |
| 1628 | 15.3 |
| 1629 | 16.2 |
| 1630 | 4.0 |
| 1631 | 6.5 |
| 1632 | 10.0 |
| 1633 | 4.6 |
| 1634 | 2.2 |
| 1635 | 45.4 |
| 1636 | 292.0 |
| 1637 | 288.0 |
| 1638 | 61.3 |
| 1639 | 273.0 |
| 1640 | 277.0 |
| 1641 | 168.0 |
| 1642 | 289.0 |
| 1643 | 34.4 |
| 1644 | 184.0 |
| 1645 | 319.0 |
| 1646 | 2.3 |
| 1647 | 2.7 |
| 1648 | 2.3 |
| 1649 | 67.2 |
| 1650 | 10.3 |
| 1651 | 3.2 |
| 1652 | 112.0 |
| 1653 | 266.0 |
| 1654 | 221.0 |
| 1655 | 238.0 |
| 1656 | 12.5 |
| 1657 | 3.4 |
| 1658 | 3.2 |
| 1659 | 4.7 |
| 1660 | 1.3 |
| 1661 | 3.4 |
| 1662 | 4.0 |
| 1663 | 1.1 |
| 1664 | 2.6 |
| 1665 | 3.8 |
| 1666 | 0.8 |
| 1667 | 46.6 |
| 1668 | 23.6 |
| 1669 | 88.8 |
| 1670 | 17.1 |
| 1671 | 149.0 |
| 1672 | 151.0 |
| 1673 | 56.1 |
| 1674 | 21.0 |
| 1675 | 12.0 |
| 1676 | 81.8 |
| 1677 | 353.0 |
| 1678 | 57.7 |
| 1679 | 18.6 |
| 1680 | 146.0 |
| 1681 | 86.7 |
| 1682 | 36.7 |
| 1683 | 109.0 |
| 1684 | 721.0 |
| 1686 | 1290.0 |
| 1687 | 128.0 |
| 1688 | 86.8 |
| 1689 | 30.9 |
| 1690 | 353.0 |
| 1691 | 556.0 |
| 1692 | 208.0 |
| 1693 | 43.6 |
| 1694 | 1.2 |
| 1695 | 4.7 |
| 1696 | 0.9 |
| 1697 | 0.5 |
| 1698 | 1.2 |
| 1699 | 11.3 |
| 1700 | 38.6 |
| 1701 | 165.0 |
| 1702 | 140.0 |
| 1703 | 387.0 |
| 1704 | 110.0 |
| 1705 | 172.0 |
| 1706 | 616.0 |
| 1707 | 124.0 |
| 1708 | 147.0 |
| 1709 | 28.1 |
| 1710 | 3.8 |
| 1711 | 4.4 |
| 1712 | 6.5 |
| 1713 | 5.0 |
| 1714 | 6.4 |
| 1715 | 1.5 |
| 1716 | 3.0 |
| 1717 | 2.5 |

¹DLD1 cells used for ATP level measurements, all IC₅₀ values were standardized to cytochalasin B IC₅₀ values;

TABLE 2
Measured IC₅₀ values of compounds regarding fructose induced
ATP increase (GLUT2 inhibition)

| | IC₅₀ |
|---|---|
| Example | [nM]¹ |
| 9 | 18371 |
| 15 | 85363 |
| 126 | 85363 |
| 240 | 10800 |
| 352 | 45100 |
| 360 | 20200 |
| 388 | 51200 |
| 415 | 18400 |
| 1571 | 36100 |
| 1558 | 51200 |

### Biological Assay: Glucose Uptake Assay

Cells (e.g. H460 or CHO-K1) were cultured under standard conditions. 10000 cells per well were seeded in clear 96 well tissue culture isoplate plates and cultured overnight (PerkinElmer, 1450-516) under standard conditions. Culture medium was removed and cells were washed two times with 100 μL KRP buffer and then incubated for 45 minutes at 37° C. (KRP buffer: 10 mM sodium hydrogen phosphate, 130 mM sodium chloride, 5 mM potassium chloride, 1.3 mM magnesium sulfate, 1.3 mM calcium chloride (pH 7.5), 50 mM HEPES (pH 7.5), 4.7 mM potassium chloride, 1.25 mM magnesium sulfate, 1.25 mM calcium chloride) each. KRP wash buffer was removed and compound 126 (diluted in KRP buffer) was added and incubated for 30 minutes at 37° C. 200 nM radioligand (radioligand 2[1,2] 3H-Deoxy D-Glucose in KRP buffer) were added and incubated for 5 minutes at room temperature. The supernatant was removed and cells were washed with 100 μL ice-cold KRP for two times each. 25 μL of Lysis buffer (1% Triton-X, 0.5N sodium hydroxide) were added and incubated at room temperature for 5 minutes. 75 μL scintillation solution (Microscint-20, PerkinElmer) were added and the plates were shaken for 1 minute. The plates were incubated for 3 h at room temperature and the counts were determined by using a Wallace MicroBeta counter (60 seconds per well).

TABLE 3
Measured IC₅₀ values of example 126 regarding GLUT1
inhibition in different cell lines

| | | IC₅₀ |
|---|---|---|
| Example | Cell line | [nM] |
| 126 | H460 | 15.4 |

Biological assay: Proliferation Assay

Cultivated tumor cells (MCF7, hormone dependent human mammary carcinoma cells, ATCC HTB22; NCI-H460, human non-small cell lung carcinoma cells, ATCC HTB-177; DU 145, hormone-independent human prostate carcinoma cells, ATCC HTB-81; HeLa-MaTu, human cervical carcinoma cells, EPO-GmbH, Berlin; HeLa-MaTu-ADR, multidrug-resistant human cervical carcinoma cells, EPO-GmbH, Berlin; HeLa human cervical tumor cells, ATCC CCL-2; B16F10 mouse melanoma cells, ATCC CRL-6475) were plated at a density of 5000 cells/well (MCF7, DU145, HeLa-MaTu-ADR), 3000 cells/well (NCI-H460, HeLa-MaTu, HeLa), or 1000 cells/well (B16F10) in a 96-well multititer plate in 200 μL of their respective growth medium supplemented 10% fetal calf serum. After 24 hours, the cells of one plate (zero-point plate) were stained with crystal violet (see below), while the medium of the other plates was replaced by fresh culture medium (200 μL), to which the test substances were added in various concentrations (0 μM, as well as in the range of 0.01-30 μM; the final concentration of the solvent dimethyl sulfoxide was 0.5%). The cells were incubated for 4 days in the presence of test substances. Cell proliferation was determined by staining the cells with crystal violet: the cells were fixed by adding 20 μL/measuring point of an 11% glutaric aldehyde solution for 15 minutes at room temperature. After three washing cycles of the fixed cells with water, the plates were dried at room temperature. The cells were stained by adding 100 μL/measuring point of a 0.1% crystal violet solution (pH 3.0). After three washing cycles of the stained cells with water, the plates were dried at room temperature. The dye was dissolved by adding 100 μL/measuring point of a 10% acetic acid solution. The extinction was determined by photometry at a wavelength of 595 nm. The change of cell number, in percent, was calculated by normalization of the measured values to the extinction values of the zero-point plate (=0%) and the extinction of the untreated (0 μm) cells (=100%). The IC₅₀ values were determined by means of a 4 parameter fit.

### Determination of Metabolic Stability In Vitro

(including calculation of hepatic in vivo blood clearance (CL) and of maximal oral bioavailability (Fₘₐₓ))

The metabolic stability of test compounds in vitro was determined by incubating them at 1 μM with a suspension Liver microsomes in 100 mM phosphate buffer, pH7.4 (NaH₂PO₄×H₂O+Na₂HPO₄×2H₂O) at a protein concentration of 0.5 mg/mL and at 37° C. The reaction was activated by adding a co-factor mix containing 1.2 mg NADP, 3 IU glucose-6-phosphate dehydrogenase, 14.6 mg glucose-6-phosphate and 4.9 mg MgCl₂ in phosphate buffer, pH 7.4. Organic solvent in the incubations was limited to <0.2% dimethylsulfoxide (DMSO) and <1% methanol. During incubation, the microsomal suspensions were continuously shaken and aliquots were taken at 2, 8, 16, 30, 45 and 60 min, to which equal volumes of cold methanol were immediately added. Samples were frozen at −20° C. over night, subsequently centrifuged for 15 minutes at 3000 rpm and the supernatant was analyzed with an Agilent 1200 HPLC-system with LCMS/MS detection.

The half-life of a test compound was determined from the concentration-time plot. From the half-life the intrinsic clearances were calculated. Together with the additional parameters liver blood flow, specific Liver weight and microsomal protein content the hepatic in vivo blood clearance (CL) and the maximal oral bioavailability (Fₘₐₓ) were calculated for the different species. The following parameter values were used: Liver blood flow—1.3 L/h/kg (human), 2.1 L/h/kg (dog), 4.2 L/h/kg (rat); specific liver weight—21 g/kg (human), 39 g/kg (dog), 32 g/kg (rat); microsomal protein content—40 mg/g.

With the described assay only phase-I metabolism of microsomes is reflected, e.g. typically oxidoreductive reactions by cytochrome P450 enzymes and flavin mono-oxygenases (FMO) and hydrolytic reactions by esterases (esters and amides).

### LITERATURE

Liu H, Hu Y P, Savarai N, Priebe W, Lampadis T. Hypersensitization of tumor cells to glycolytic inhibitors. Biochemistry. 2001; 40:5542-5547.
Ulanovskaya O, Janjic J, Matsumoto K, Schumacker P T, Kron S J, Kozmin S A. Synthesis enables identification of the cellular target of Leucascandrolide A and neopettotide. Nat Chem Biol. 2008; 4:418-424.
Ulanovskaya O, Jiayue Cui, Stephen J. Kron, and Sergey A. Kozmin. A pairwise chemical genetic screen identifies new inhibitors of glucose transport. Chem Biol. 2011 Feb. 25; 18(2): 222-230.

LENGTHY TABLES
The patent application contains a lengthy table section. A copy of the table is available in electronic form from the USPTO web site (http://seqdata.uspto.gov/?pageRequest=docDetail&DocID=US20170037028A1). An electronic copy of the table will also be available from the USPTO upon request and payment of the fee set forth in 37 CFR 1.19(b)(3).

1. A compound of formula (I): 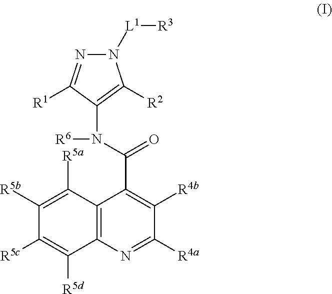
in which:
R¹ represents a C₁-C₃-alkyl-, halo-C₁-C₃-alkyl-, cyano-, —C(═O)O—R¹⁰ or —C(═O)N(R^{10a})R^{10b} group;
R² represents a C₁-C₃-alkyl-, halo-C₁-C₃-alkyl-, cyano-, —C(═O)O—R¹⁰ or —C(═O)N(R^{10a})R^{10b} group;
R³ represents a group selected from: aryl-, heteroaryl-, C₅-C₆-cycloalkyl-, and 5- to 6-membered heterocycloalkyl-;
wherein said 5- to 6-membered heterocycloalkyl- group is optionally benzocondensed;
wherein said aryl-, heteroaryl-, C₅-C₆-cycloalkyl-, and 5- to 6-membered heterocycloalkyl- group is optionally substituted, one or more times, identically or differently, with -(L²)ₚ-R⁷;
and wherein two -(L²)ₚ-R⁷ groups, if being present ortho to each other on an aryl- or heteroaryl- group optionally form a bridge selected from:
*—C₃-C₈-alkylene-*, *—O(CH₂)₂O—*, *—O(CH₂)O—*, *—O(CF₂)O—*, *—CH₂C(R^{10a})(R^{10b})O—*, *—C(═O)N(R^{10a})CH₂—*, *—N(R^{10a})C(═O)CH₂O—*, *—NHC(═O)NH—*; wherein each * represents the point of attachment to said aryl- or heteroaryl- group;
R^{4a} represents a hydrogen atom or a halogen atom or a group selected from:
cyano-, hydroxy-, C₁-C₃-alkyl-, halo-C₁-C₃-alkyl-, C₁-C₃-alkoxy-, halo-C₁-C₃-alkoxy-, C₃-C₇-cycloalkyl-, 4- to 7-membered heterocycloalkyl-, —C(═O)—OR¹⁰, —C(═O)N(R^{10a})R^{10b},
—C(═O)—N(R^{10a})—S(═O)₂—R¹⁰, —SR¹⁰, —S(═O)—R¹⁰, —S(═NR¹¹)—R¹⁰, —S(═O)₂—R¹⁰, —S(═O)₂—N(R^{10a})R^{10b}, —S(═O)(═NR¹¹)—R¹⁰, —N(R^{10a})R^{10b};
R^{4b} represents a hydrogen atom or a group selected from: C₁-C₃-alkoxy-, C₁-C₃-alkyl-, cyano-;
or
R^{4a} and together R^{4b} form a —C₃-C₅-alkylene- group;
R^{5a}, R^{5b}, R^{5c}, R^{5d}
independently from each other represent a hydrogen atom, a halogen atom or a group selected from:
cyano-, —NO₂, C₁-C₃-alkyl-, halo-C₁-C₃-alkyl-, C₁-C₃-alkoxy-, halo-C₁-C₃-alkoxy-, phenyl-, heteroaryl-,
—C(═O)R¹⁰, —C(═O)N(H)R¹⁰, —C(═O)N(R^{10a})R^{10b}, —C(═O)O—R¹⁰, —N(R^{10a})R^{10b}, —N(H)C(═O)R¹⁰, —N(R^{10a})C(═O)R^{10b}, —N(H)C(═O)N(R^{10a})R^{10b}, —N(R^{10a})C(═O)N(R^{10b})R^{10c}, —N(R^{10a})C(═O)C(═O)N(R^{10b})R^{10c}, —N(H)C(═O)OR¹⁰, —N(R^{10a})C(═O)OR^{10b}, —N(H)S(═O)₂R¹⁰, —N (R^{10a})S(═O)₂R^{10b}, —OR¹⁰, —O(C═O)R¹⁰, —O(C═O)N(R^{10a})R^{10b}, —O(C═O)OR¹⁰, —SR¹⁰, —S(═O)R¹⁰, —S(═O)₂R¹⁰, —S(═O)₂N(H)R¹⁰, —S(═O)₂N(R^{10a})R^{10b} or —S(═O)(═NR^{10a})R^{10b}, said phenyl- or heteroaryl- group being optionally substituted one or more times, identically or differently, with a group selected from:
halo-, cyano-, C₁-C₃-alkyl-, halo-C₁-C₃-alkyl-, C₁-C₃-alkoxy-;
R⁶ represents a hydrogen atom or group selected from: C₁-C₃-alkyl-, C₁-C₃-alkoxy-(L²)-, hydroxy-C₁-C₃-alkyl-, aryl-(L²)-, heteroaryl-(L²)-;
R⁷ represents a group selected from: oxo, C₁-C₆-alkyl-, C₃-C₇-cycloalkyl-, 4- to 7-membered heterocycloalkyl-, halo-C₁-C₄-alkyl-, hydroxy-C₁-C₄-alkyl-, cyano-C₁-C₄-alkyl-, C₂-C₄-alkenyl-, C₂-C₄-alkynyl-, C₁-C₄-alkoxy-, halo-C₁-C₄-alkoxy-, —OH, —CN, halo-, —C(═O)R⁸, —C(═O)—O—R⁸, —C(═O)N(R^{8a})R^{8b}, —N(R^{10a})R^{10b}, —S(═O)₂R⁸, —S(═O)(═NR¹¹)—R¹⁰, phenyl-,
5- to 6-membered heteroaryl-;
R⁸ represents a hydrogen atom or a C₁-C₆-alkyl-, halo-C₁-C₃-alkyl-, cyano-C₁-C₄-alkyl-, C₁-C₃-alkoxy-C₁-C₃-alkyl-, C₃-C₇-cycloalkyl-, phenyl-, 5- to 6-membered heteroaryl- or benzyl- group;
R^{8a}, R^{8b}
represent, independently from each other, a hydrogen atom, or a C₁-C₁₀-alkyl-, C₃-C₇-cycloalkyl-, (C₃-C₇-cycloalkyl)-(L³)-, C₃-C₆-alkenyl-, C₃-C₆-alkynyl-, 4- to 10-membered heterocycloalkyl-,
(4- to 10-membered heterocycloalkyl)-(L³)-, phenyl-, heteroaryl-, phenyl-(L³)-, (phenyl)-O-(L³)-, heteroaryl-(L³)-, or
(aryl)-(4- to 10-membered heterocycloalkyl)- group;
said C₁-C₁₀-alkyl-, C₃-C₇-cycloalkyl-, (C₃-C₇-cycloalkyl)-(L³)-, C₃-C₆-alkenyl-, C₃-C₆-alkynyl-, 4- to 10-membered heterocycloalkyl-, (4- to 10-membered heterocycloalkyl)-(L³)-, phenyl-, heteroaryl-, phenyl-(L³)-, (phenyl)-O-(L³)-, heteroaryl-(L³)-, and (aryl)-(4- to 10-membered heterocycloalkyl)- group being optionally substituted one or more times, identically or differently, with R⁹;
or
R^{8a} and R^{8b}, together with the nitrogen atom they are attached to,
represent a 4- to 10-membered heterocycloalkyl-group, said 4- to 10-membered heterocycloalkyl- group being optionally substituted one or more times, identically or differently, with R⁹;
R⁹ represents a halogen atom, or a oxo, C₁-C₃-alkyl-, halo-C₁-C₃-alkyl-, hydroxy-C₁-C₃-alkyl-,
—CN, —C(═O)R¹⁰, —C(═O)N(H)R¹⁰, —C(═O)N(R^{10a})R^{10b}, —C(═O)O—R¹⁰, —N(R^{10a})R^{10b}, —N O₂, —N(H)C(═O)R¹⁰, —N(R^{10a})C(═O)R^{10b}, —N(H)C(═O)N(R^{10a})R^{10b}, —N(R^{10a})C(═O)N(R^{10b})R^{10c}, —N(H)C(═O)OR¹⁰, —N(R^{10a})C(═O)OR^{10b}, —N(H)S(═O)₂R¹⁰, —N(R^{10a})S(═O)₂R^{10b}, —O R¹⁰, —O(C═O)R¹⁰, —O(C═O)N(R^{10a})R^{10b}, —O(C═O)OR¹⁰, —SR¹⁰, —S(═O)R¹⁰, —S(═O)₂R¹⁰, —S(═O)₂N(H)R¹⁰, —S(═O)₂N(R^{10a})R^{10b}, —S(═O)(═NR^{10a})R^{10b} or a tetrazolyl- group;
or
two R⁹ groups present ortho to each other on a phenyl- or heteroaryl- ring form a bridge selected from: *—C₃-C₅-alkylene-*, *—O(CH₂)₂O—*, *—O(CH₂)O—*, *—O(CF₂)O—*, *—CH₂C(R^{10a})(R^{10b})O—*, *—C(═O)N(R^{10a})CH₂—*, *—N(R^{10a})C(═O)CH₂O—*, *—NHC(═O)NH—*; wherein each * represents the point of attachment to said phenyl- or heteroaryl- ring;
R¹⁰, R^{10a}, R^{10b}, R^{10c}
represent, independently from each other, a hydrogen atom or a group selected from: C₁-C₃-alkyl-, halo-C₁-C₃-alkyl-, hydroxy-C₁-C₃-alkyl-, C₁-C₃-alkoxy-C₁-C₃-alkyl-, C₃-C₇-cycloalkyl-, said C₁-C₃-alkyl- group being optionally substituted once with —N(R¹²)R^{12a};
or
R^{10a} and R^{10b}, together with the nitrogen atom they are attached to,
represent a 4- to 7-membered heterocycloalkyl- group, said 4- to 7-membered heterocycloalkyl-group being optionally substituted one or more times, identically or differently, with R¹³;
R¹¹ represents a hydrogen atom or a cyano-, C₁-C₃-alkyl-, —C(═O)R¹⁰, —C(═O)N(H)R¹⁰, —C(═O)N(R^{10a})R^{10b} or —C(═O)O—R¹⁰ group;
R¹², R^{12a}
represent, independently from each other, a hydrogen atom or a C₁-C₃-alkyl-group,
or,
R¹², R^{12a},
together with the nitrogen atom they are attached to, represent a 4- to 7-membered heterocycloalkyl- group;
R¹³ represents a halogen atom or a cyano, hydroxy, oxo, C₁-C₃-alkyl-, trifluoromethyl-, —C(═O)R¹⁰ or —C(═O)O—R¹⁰ group;
L¹ represents a group selected from: —C₁-C₄-alkylene-, —CH₂—CH═CH—, —C(phenyl)(H)—, —CH₂—CH₂—O—, —CH₂—C(═O)—N(H)—, —CH₂—C(═O)—N(R^{10a})—;
L² represents a group selected from: —CH₂—, —CH₂—CH₂—, —CH₂—CH₂—CH₂—;
L³ represents a —C₁-C₆-alkylene- group;
p is an integer of 0 or 1;
or a tautomer, a stereoisomer, an N-oxide, a hydrate, a solvate, or a salt thereof, or a mixture of same.

2. A compound according to claim 1, wherein at least one of R¹ and R² is different from iso-propyl-.

3. A compound according to claim 1, wherein R¹ represents a C₁-C₃-alkyl-, trifluoromethyl- or cyano- group, and wherein R² represents a methyl-, ethyl- or trifluoromethyl- group.

4. A compound according to claim 1, wherein R³ represents a group selected from: phenyl- or 5- to 6-membered heteroaryl-;
wherein said phenyl- or 5- to 6-membered heteroaryl- group is optionally substituted, one or more times, identically or differently, with -(L²)ₚ-R⁷,
or wherein R³ represents a group 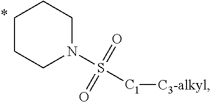
wherein * represents the point of attachment to the rest of the molecule.

5. A compound according to claim 1, wherein R^{4a} represents a group selected from: C₁-C₃-alkyl-, fluoro-C₁-C₃-alkyl-, C₁-C₃-alkoxy-, C₃-C₅-cycloalkyl, —C(═O)N(R^{10a})R^{10b}, —SR¹⁰, —S(═O)—R¹⁰, —S(═NR¹¹)—R¹⁰, —S(═O)₂—R¹⁰, —S(═O)₂—N(R^{10a})R^{10b}, —N(R^{10a})R^{10b}.

6. A compound according to claim 1, wherein R^{4a} represents a group selected from: iso-propyl-, trifluoromethyl-, methoxy-, cyclopropyl-,
—C(═O)—NH₂.

7. A compound according to claim 1, wherein R^{4b} represents a hydrogen atom.

8. A compound according to claim 1, wherein R^{5a}, R^{5b}, R^{5c}, and R^{5d} independently from each other represent a hydrogen atom, a halogen atom or a group selected from:
cyano-, C₁-C₃-alkyl-, C₁-C₃-alkoxy-, —N(R^{10a})R^{10b}, —OR¹⁰.

9. A compound according to claim 1, wherein R⁶ represents a hydrogen atom.

10. A compound according to claim 1, wherein R⁷ represents a group selected from: C₁-C₃-alkyl-, cyclopropyl-, trifluoromethyl-, C₁-C₃-alkoxy-, trifluoromethoxy-, —CN, fluoro-, chloro-, —C(═O)—C₁-C₃-alkyl, —C(═O)N(R^{8a})R^{8b}, —S(═O)₂)—C₁-C₃-alkyl.

11. A compound according to claim 1, wherein R⁸ represents a hydrogen atom or a C₁-C₆-alkyl- group.

12. A compound according to claim 1, wherein R^{8a} and R^{8b}, independently from each other, represent a hydrogen atom, or a C₁-C₄-alkyl-, C₃-C₅-cycloalkyl-, 4- to 7-membered heterocycloalkyl-, (4- to 7-membered heterocycloalkyl)-(L³)-, phenyl- or heteroaryl-(L³)- group; said C₁-C₄-alkyl-, C₃-C₅-cycloalkyl-, 4- to 7-membered heterocycloalkyl-, (4- to 7-membered heterocycloalkyl)-(L³)-, phenyl- or heteroaryl-(L³)- group being optionally substituted one or more times, identically or differently, with R⁹;
or wherein R^{8a} and R^{8b}, together with the nitrogen atom they are attached to, represent a 4- to 7-membered heterocycloalkyl-group.

13. A compound according to claim 1, wherein R⁹ represents a halogen atom, or a C₁-C₃-alkyl-, fluoro-C₁-C₃-alkyl-, hydroxy-C₁-C₃-alkyl-,
—CN, —C(═O)R¹⁰, —C(═O)N(H)R¹⁰, —C(═O)N(R^{10a})R^{10b}, —N(R^{10a})R^{10b}, —N(H)C(═O)R¹⁰, —N(R^{10a}) C(═O)R^{10b}, —OR¹⁰ group.

14. A compound according to claim 1, wherein L¹ represents a group selected from: —CH₂—, —C(CH₃)(H)—.

15. A compound according to claim 1, which is selected from the group consisting of:
2-methoxy-N-[1-(4-methoxybenzyl)-3,5-dimethyl-1H-pyrazol-4-yl]quinoline-4-carboxamide,
6-bromo-N-[1-(4-fluorobenzyl)-3,5-dimethyl-1H-pyrazol-4-yl]-2-(propan-2-yl)quinoline-4-carboxamide,
6-bromo-N-[1-(4-methoxybenzyl)-3,5-dimethyl-1H-pyrazol-4-yl]-2-(trifluoromethyl)quinoline-4-carboxamide,
N-[1-(4-methoxybenzyl)-3,5-dimethyl-1H-pyrazol-4-yl]-2,6-dimethylquinoline-4-carboxamide,
6-bromo-N-[1-(4-cyanobenzyl)-3,5-dimethyl-1H-pyrazol-4-yl]-2-(trifluoromethyl)quinoline-4-carboxamide,
6-bromo-N-[1-(2-cyanobenzyl)-3,5-dimethyl-1H-pyrazol-4-yl]-2-(trifluoromethyl)quinoline-4-carboxamide,
2-cyclopropyl-N-[1-(4-fluorobenzyl)-3,5-dimethyl-1H-pyrazol-4-yl]-6-methylquinoline-4-carboxamide,
6,8-dichloro-N-[1-(4-methoxybenzyl)-3,5-dimethyl-1H-pyrazol-4-yl]-2-(trifluoromethyl)quinoline-4-carboxamide,
6-bromo-N-[1-(4-fluorobenzyl)-3,5-dimethyl-1H-pyrazol-4-yl]-2-(trifluoromethyl)quinoline-4-carboxamide,
2-cyclopropyl-6-fluoro-N-[1-(4-fluorobenzyl)-3,5-dimethyl-1H-pyrazol-4-yl]quinoline-4-carboxamide,
6,8-dichloro-N-[1-(4-cyanobenzyl)-3,5-dimethyl-1H-pyrazol-4-yl]-2-(trifluoromethyl)quinoline-4-carboxamide,
6-bromo-2-cyclopropyl-N-[1-(4-fluorobenzyl)-3,5-dimethyl-1H-pyrazol-4-yl]quinoline-4-carboxamide,
6-bromo-N-[1-(2,6-difluorobenzyl)-3,5-dimethyl-1H-pyrazol-4-yl]-2-(trifluoromethyl)quinoline-4-carboxamide,
6-chloro-N-[1-(4-fluorobenzyl)-3,5-dimethyl-1H-pyrazol-4-yl]-2-(trifluoromethyl)quinoline-4-carboxamide,
N-[1-(4-cyanobenzyl)-3,5-dimethyl-1H-pyrazol-4-yl]-2-methoxyquinoline-4-carboxamide,
8-bromo-2-cyclopropyl-N-[1-(4-fluorobenzyl)-3,5-dimethyl-1H-pyrazol-4-yl]quinoline-4-carboxamide,
N-[1-(2,4-difluorobenzyl)-3,5-dimethyl-1H-pyrazol-4-yl]-2-methoxyquinoline-4-carboxamide,
8-bromo-N-[1-(4-fluorobenzyl)-3,5-dimethyl-1H-pyrazol-4-yl]-2-(trifluoromethyl)quinoline-4-carboxamide,
2-cyclopropyl-N-[1-(4-fluorobenzyl)-3,5-dimethyl-1H-pyrazol-4-yl]quinoline-4-carboxamide,
6,8-dichloro-N-[1-(2,6-difluorobenzyl)-3,5-dimethyl-1H-pyrazol-4-yl]-2-(trifluoromethyl)quinoline-4-carboxamide,
8-bromo-N-[1-(4-fluorobenzyl)-3,5-dimethyl-1H-pyrazol-4-yl]-6-methyl-2-(trifluoromethyl)quinoline-4-carboxamide,
5,6-dichloro-N-[1-(4-fluorobenzyl)-3,5-dimethyl-1H-pyrazol-4-yl]-2-(trifluoromethyl)quinoline-4-carboxamide,
N-{3,5-dimethyl-1-[4-(trifluoromethoxy)benzyl]-1H-pyrazol-4-yl}-2-methoxyquinoline-4-carboxamide,
6-bromo-N-[3,5-dimethyl-1-(2-methylbenzyl)-1H-pyrazol-4-yl]-2-(trifluoromethyl)quinoline-4-carboxamide,
6,8-dichloro-N-[1-(4-fluorobenzyl)-3,5-dimethyl-1H-pyrazol-4-yl]-2-(trifluoromethyl)quinoline-4-carboxamide,
5-fluoro-N-[1-(4-fluorobenzyl)-3,5-dimethyl-1H-pyrazol-4-yl]-2-(trifluoromethyl)quinoline-4-carboxamide,
N-[1-(4-fluorobenzyl)-3,5-dimethyl-1H-pyrazol-4-yl]-2-methoxyquinoline-4-carboxamide,
6,8-dichloro-N-[3,5-dimethyl-1-(3-methylbenzyl)-1H-pyrazol-4-yl]-2-(trifluoromethyl)quinoline-4-carboxamide,
7-fluoro-N-[1-(4-fluorobenzyl)-3,5-dimethyl-1H-pyrazol-4-yl]-2-(trifluoromethyl)quinoline-4-carboxamide,
6-fluoro-N-[1-(4-fluorobenzyl)-3,5-dimethyl-1H-pyrazol-4-yl]-2-(trifluoromethyl)quinoline-4-carboxamide,
6,8-dichloro-N-[3,5-dimethyl-1-(4-methylbenzyl)-1H-pyrazol-4-yl]-2-(trifluoromethyl)quinoline-4-carboxamide,
6-bromo-N-[1-(4-fluorobenzyl)-5-methyl-3-(trifluoromethyl)-1H-pyrazol-4-yl]-2-(trifluoromethyl)quinoline-4-carboxamide,
N-(1-benzyl-3,5-dimethyl-1H-pyrazol-4-yl)-6-bromo-2-(trifluoromethyl)quinoline-4-carboxamide,
5-bromo-N-[1-(4-fluorobenzyl)-3,5-dimethyl-1H-pyrazol-4-yl]-2-(trifluoromethyl)quinoline-4-carboxamide,
6,8-dichloro-N-[1-(3,4-difluorobenzyl)-3,5-dimethyl-1H-pyrazol-4-yl]-2-(trifluoromethyl)quinoline-4-carboxamide,
2-bromo-N-[1-(4-fluorobenzyl)-3,5-dimethyl-1H-pyrazol-4-yl]quinoline-4-carboxamide,
7-bromo-N-[1-(4-fluorobenzyl)-3,5-dimethyl-1H-pyrazol-4-yl]-1,2,3,4-tetrahydroacridine-9-carboxamide,
6,8-dichloro-N-[1-(4-fluorobenzyl)-5-methyl-3-(trifluoromethyl)-1H-pyrazol-4-yl]-2-(trifluoromethyl)quinoline-4-carboxamide,
6,7-difluoro-N-[1-(4-fluorobenzyl)-3,5-dimethyl-1H-pyrazol-4-yl]-2-(trifluoromethyl)quinoline-4-carboxamide,
2-cyclopropyl-8-fluoro-N-[1-(4-fluorobenzyl)-3,5-dimethyl-1H-pyrazol-4-yl]quinoline-4-carboxamide,
6-bromo-N-[1-(2,4-difluorobenzyl)-3,5-dimethyl-1H-pyrazol-4-yl]-2-(trifluoromethyl)quinoline-4-carboxamide,
N-[1-(2,6-difluorobenzyl)-3,5-dimethyl-1H-pyrazol-4-yl]-2-methoxyquinoline-4-carboxamide,
6-bromo-N-[1-(4-fluorobenzyl)-3,5-dimethyl-1H-pyrazol-4-yl]-2-methylquinoline-4-carboxamide,
N-[1-(4-cyanobenzyl)-3,5-dimethyl-1H-pyrazol-4-yl]-2,6-dimethylquinoline-4-carboxamide,
N-[3,5-dimethyl-1-(4-methylbenzyl)-1H-pyrazol-4-yl]-2,6-dimethylquinoline-4-carboxamide,
2-cyclopropyl-5-fluoro-N-[1-(4-fluorobenzyl)-3,5-dimethyl-1H-pyrazol-4-yl]quinoline-4-carboxamide,
6-bromo-N-[1-(3,4-difluorobenzyl)-3,5-dimethyl-1H-pyrazol-4-yl]-2-(trifluoromethyl)quinoline-4-carboxamide,
6,8-dichloro-N-[1-(2,4-difluorobenzyl)-3,5-dimethyl-1H-pyrazol-4-yl]-2-(trifluoromethyl)quinoline-4-carboxamide,
6-bromo-N-[3,5-dimethyl-1-(2,4,6-trifluorobenzyl)-1H-pyrazol-4-yl]-2-(trifluoromethyl)quinoline-4-carboxamide,
N-[3,5-dimethyl-1-(2,4,6-trifluorobenzyl)-1H-pyrazol-4-yl]-2-methoxyquinoline-4-carboxamide,
N-[3,5-dimethyl-1-(2-methylbenzyl)-1H-pyrazol-4-yl]-2-methoxyquinoline-4-carboxamide,
6-bromo-N-[3,5-dimethyl-1-(3-methylbenzyl)-1H-pyrazol-4-yl]-2-(trifluoromethyl)quinoline-4-carboxamide,
8-bromo-N-[1-(4-fluorobenzyl)-3,5-dimethyl-1H-pyrazol-4-yl]-2-methylquinoline-4-carboxamide,
6-bromo-N-[3,5-dimethyl-1-(4-methylbenzyl)-1H-pyrazol-4-yl]-2-(trifluoromethyl)quinoline-4-carboxamide,
N-[1-(3,4-difluorobenzyl)-3,5-dimethyl-1H-pyrazol-4-yl]-2-methoxyquinoline-4-carboxamide,
6-bromo-N-{3,5-dimethyl-1-[4-(trifluoromethoxy)benzyl]-1H-pyrazol-4-yl}-2-(trifluoromethyl)quinoline-4-carboxamide,
6,8-dichloro-N-[3,5-dimethyl-1-(2,4,6-trifluorobenzyl)-1H-pyrazol-4-yl]-2-(trifluoromethyl)quinoline-4-carboxamide,
N-{3,5-dimethyl-1-[4-(trifluoromethoxy)benzyl]-1H-pyrazol-4-yl}-2,6-dimethylquinoline-4-carboxamide,
N-(1-benzyl-3,5-dimethyl-1H-pyrazol-4-yl)-6,8-dichloro-2-(trifluoromethyl)quinoline-4-carboxamide,
6,8-dichloro-N-{3,5-dimethyl-1-[4-(trifluoromethoxy)benzyl]-1H-pyrazol-4-yl}-2-(trifluoromethyl)quinoline-4-carboxamide,
N-[3,5-dimethyl-1-(4-methylbenzyl)-1H-pyrazol-4-yl]-2-methoxyquinoline-4-carboxamide,
N-[1-(4-fluorobenzyl)-3,5-dimethyl-1H-pyrazol-4-yl]-2,6-dimethylquinoline-4-carboxamide,
N-[1-(4-fluorobenzyl)-3,5-dimethyl-1H-pyrazol-4-yl]-6-(trifluoromethoxy)-2-(trifluoromethyl)quinoline-4-carboxamide,
N-[1-(3,4-difluorobenzyl)-3,5-dimethyl-1H-pyrazol-4-yl]-2,6-dimethylquinoline-4-carboxamide,
N-(1-benzyl-3,5-dimethyl-1H-pyrazol-4-yl)-2-methoxyquinoline-4-carboxamide,
6,8-dichloro-N-[3,5-dimethyl-1-(pyridin-2-ylmethyl)-1H-pyrazol-4-yl]-2-(trifluoromethyl)quinoline-4-carboxamide,
6,8-dichloro-N-[1-(3-methoxybenzyl)-3,5-dimethyl-1H-pyrazol-4-yl]-2-(trifluoromethyl)quinoline-4-carboxamide,
6,8-dichloro-N-[1-(2-cyanobenzyl)-3,5-dimethyl-1H-pyrazol-4-yl]-2-(trifluoromethyl)quinoline-4-carboxamide,
2-ethyl-N-[1-(4-fluorobenzyl)-3,5-dimethyl-1H-pyrazol-4-yl]quinoline-4-carboxamide,
6-bromo-N-[3,5-dimethyl-1-(pyridin-2-ylmethyl)-1H-pyrazol-4-yl]-2-(trifluoromethyl)quinoline-4-carboxamide,
6-bromo-N-[1-(3-cyanobenzyl)-3,5-dimethyl-1H-pyrazol-4-yl]-2-(trifluoromethyl)quinoline-4-carboxamide,
6-bromo-N-[1-(3-fluorobenzyl)-3,5-dimethyl-1H-pyrazol-4-yl]-2-(trifluoromethyl)quinoline-4-carboxamide,
N-[1-(4-fluorobenzyl)-5-methyl-3-(trifluoromethyl)-1H-pyrazol-4-yl]-2,6-dimethylquinoline-4-carboxamide,
6-bromo-N-[1-(2-fluorobenzyl)-3,5-dimethyl-1H-pyrazol-4-yl]-2-(trifluoromethyl)quinoline-4-carboxamide,
N-[1-(4-fluorobenzyl)-3,5-dimethyl-1H-pyrazol-4-yl]-2-hydroxy-6-methoxyquinoline-4-carboxamide,
N-[3,5-dimethyl-1-(3-methylbenzyl)-1H-pyrazol-4-yl]-2-methoxyquinoline-4-carboxamide,
N-[1-(2-fluorobenzyl)-3,5-dimethyl-1H-pyrazol-4-yl]-2-methoxyquinoline-4-carboxamide,
2-cyano-N-[1-(4-fluorobenzyl)-3,5-dimethyl-1H-pyrazol-4-yl]quinoline-4-carboxamide,
2-cyclopropyl-N-[1-(4-fluorobenzyl)-3,5-dimethyl-1H-pyrazol-4-yl]-6-(trifluoromethyl)quinoline-4-carboxamide,
N-[1-(2-cyanobenzyl)-3,5-dimethyl-1H-pyrazol-4-yl]-2-methoxyquinoline-4-carboxamide,
2-methoxy-N-[1-(3-methoxybenzyl)-3,5-dimethyl-1H-pyrazol-4-yl]quinoline-4-carboxamide,
6,8-dichloro-N-{3,5-dimethyl-1-[(3-methylpyridin-2-yl)methyl]-1H-pyrazol-4-yl}-2-(trifluoromethyl)quinoline-4-carboxamide,
6-bromo-N-{3,5-dimethyl-1-[2-(trifluoromethoxy)benzyl]-1H-pyrazol-4-yl}-2-(trifluoromethyl)quinoline-4-carboxamide,
N-[1-(2,6-difluorobenzyl)-3,5-dimethyl-1H-pyrazol-4-yl]-2,6-dimethylquinoline-4-carboxamide,
8-fluoro-N-[1-(4-fluorobenzyl)-3,5-dimethyl-1H-pyrazol-4-yl]-2-(trifluoromethyl)quinoline-4-carboxamide,
N-[1-(4-fluorobenzyl)-3,5-dimethyl-1H-pyrazol-4-yl]-2,8-dimethylquinoline-4-carboxamide,
N-[1-(3-fluorobenzyl)-3,5-dimethyl-1H-pyrazol-4-yl]-2-methoxyquinoline-4-carboxamide,
7-bromo-N-[1-(4-fluorobenzyl)-3,5-dimethyl-1H-pyrazol-4-yl]-2-(trifluoromethyl)quinoline-4-carboxamide,
6,8-dichloro-N-[3,5-dimethyl-1-(2-methylbenzyl)-1H-pyrazol-4-yl]-2-(trifluoromethyl)quinoline-4-carboxamide,
6-fluoro-N-[1-(4-fluorobenzyl)-3,5-dimethyl-1H-pyrazol-4-yl]-2-methylquinoline-4-carboxamide,
6,8-dichloro-N-[1-(3-cyanobenzyl)-3,5-dimethyl-1H-pyrazol-4-yl]-2-(trifluoromethyl)quinoline-4-carboxamide,
N-(1-benzyl-3,5-dimethyl-1H-pyrazol-4-yl)-2,6-dimethylquinoline-4-carboxamide,
6,8-dichloro-N-{3,5-dimethyl-1-[3-(trifluoromethoxy)benzyl]-1H-pyrazol-4-yl}-2-(trifluoromethyl)quinoline-4-carboxamide,
6,8-dichloro-N-[1-(2-fluorobenzyl)-3,5-dimethyl-1H-pyrazol-4-yl]-2-(trifluoromethyl)quinoline-4-carboxamide,
N-[1-(4-fluorobenzyl)-3,5-dimethyl-1H-pyrazol-4-yl]-3-methoxy-2-methylquinoline-4-carboxamide,
N-[1-(2,4-difluorobenzyl)-3,5-dimethyl-1H-pyrazol-4-yl]-2,6-dimethylquinoline-4-carboxamide,
6-bromo-N-[1-(3-methoxybenzyl)-3,5-dimethyl-1H-pyrazol-4-yl]-2-(trifluoromethyl)quinoline-4-carboxamide,
N-[1-(4-fluorobenzyl)-3,5-dimethyl-1H-pyrazol-4-yl]-2,6-bis(trifluoromethyl)quinoline-4-carboxamide,
2-chloro-N-[1-(4-fluorobenzyl)-3,5-dimethyl-1H-pyrazol-4-yl]quinoline-4-carboxamide,
7-bromo-2-cyclopropyl-N-[1-(4-fluorobenzyl)-3,5-dimethyl-1H-pyrazol-4-yl]quinoline-4-carboxamide,
N-[3,5-dimethyl-1-(3-methylbenzyl)-1H-pyrazol-4-yl]-2,6-dimethylquinoline-4-carboxamide,
8-cyano-N-[1-(4-fluorobenzyl)-3,5-dimethyl-1H-pyrazol-4-yl]-6-methyl-2-(trifluoromethyl)quinoline-4-carboxamide,
6-cyano-N-[1-(4-fluorobenzyl)-3,5-dimethyl-1H-pyrazol-4-yl]-2-(trifluoromethyl)quinoline-4-carboxamide,
6,8-dichloro-N-[1-(3-fluorobenzyl)-3,5-dimethyl-1H-pyrazol-4-yl]-2-(trifluoromethyl)quinoline-4-carboxamide,
N-[1-(4-fluorobenzyl)-3,5-dimethyl-1H-pyrazol-4-yl]-2-methylquinoline-4-carboxamide,
6,7-dichloro-N-[1-(4-fluorobenzyl)-3,5-dimethyl-1H-pyrazol-4-yl]-2-(trifluoromethyl)quinoline-4-carboxamide,
N-{3,5-dimethyl-1-[4-(trifluoromethyl)benzyl]-1H-pyrazol-4-yl}-2,6-dimethylquinoline-4-carboxamide,
2-cyclopropyl-N-[1-(4-fluorobenzyl)-3,5-dimethyl-1H-pyrazol-4-yl]-8-(trifluoromethyl)quinoline-4-carboxamide,
N-[1-(3-methoxybenzyl)-3,5-dimethyl-1H-pyrazol-4-yl]-2,6-dimethylquinoline-4-carboxamide,
N-[3,5-dimethyl-1-(pyridin-2-ylmethyl)-1H-pyrazol-4-yl]-2-methoxyquinoline-4-carboxamide,
N-[3,5-dimethyl-1-(2-methylbenzyl)-1H-pyrazol-4-yl]-2,6-dimethylquinoline-4-carboxamide,
N-[1-(2-fluorobenzyl)-3,5-dimethyl-1H-pyrazol-4-yl]-2,6-dimethylquinoline-4-carboxamide,
6-bromo-N-{3,5-dimethyl-1-[3-(trifluoromethoxy)benzyl]-1H-pyrazol-4-yl}-2-(trifluoromethyl)quinoline-4-carboxamide,
N-[1-(2-cyanobenzyl)-3,5-dimethyl-1H-pyrazol-4-yl]-2,6-dimethylquinoline-4-carboxamide,
N-{3,5-dimethyl-1-[3-(trifluoromethoxy)benzyl]-1H-pyrazol-4-yl}-2-methoxyquinoline-4-carboxamide,
6-bromo-N-{3,5-dimethyl-1-[(3-methylpyridin-2-yl)methyl]-1H-pyrazol-4-yl}-2-(trifluoromethyl)quinoline-4-carboxamide,
N-{3,5-dimethyl-1-[3-(trifluoromethoxy)benzyl]-1H-pyrazol-4-yl}-2,6-dimethylquinoline-4-carboxamide,
6-bromo-N-[1-(2-cyanobenzyl)-3,5-dimethyl-1H-pyrazol-4-yl]-2-(propan-2-yl)quinoline-4-carboxamide,
6-bromo-N⁴-[1-(4-fluorobenzyl)-3,5-dimethyl-1H-pyrazol-4-yl]quinoline-2,4-dicarboxamide,
6,7-difluoro-N⁴-[1-(4-fluorobenzyl)-3,5-dimethyl-1H-pyrazol-4-yl]quinoline-2,4-dicarboxamide,
6-chloro-7-fluoro-N-[1-(4-fluorobenzyl)-3,5-dimethyl-1H-pyrazol-4-yl]-2-(trifluoromethyl)quinoline-4-carboxamide,
N⁴-[1-(4-fluorobenzyl)-3,5-dimethyl-1H-pyrazol-4-yl]quinoline-2,4-dicarboxamide,
6-bromo-2-cyclopropyl-N-[1-(3,4-difluorobenzyl)-3,5-dimethyl-1H-pyrazol-4-yl]quinoline-4-carboxamide,
6-bromo-7-fluoro-N-[1-(4-fluorobenzyl)-3,5-dimethyl-1H-pyrazol-4-yl]-2-(trifluoromethyl)quinoline-4-carboxamide,
6-chloro-2-cyclopropyl-N-[1-(4-fluorobenzyl)-3,5-dimethyl-1H-pyrazol-4-yl]quinoline-4-carboxamide,
N⁴-[1-(4-cyanobenzyl)-5-methyl-3-(trifluoromethyl)-1H-pyrazol-4-yl]quinoline-2,4-dicarboxamide,
6,8-dichloro-N⁴-[1-(4-fluorobenzyl)-3,5-dimethyl-1H-pyrazol-4-yl]quinoline-2,4-dicarboxamide,
N⁴-[1-(3,4-difluorobenzyl)-5-methyl-3-(trifluoromethyl)-1H-pyrazol-4-yl]quinoline-2,4-dicarboxamide,
6-bromo-N-[1-(3,4-difluorobenzyl)-3,5-dimethyl-1H-pyrazol-4-yl]-2-(propan-2-yl)quinoline-4-carboxamide,
N⁴-[1-(4-fluorobenzyl)-5-methyl-3-(trifluoromethyl)-1H-pyrazol-4-yl]quinoline-2,4-dicarboxamide,
methyl 4-{[4-({[6-bromo-2-(trifluoromethyl)quinolin-4-yl]carbonyl}amino)-3,5-dimethyl-1H-pyrazol-1-yl]methyl}benzoate,
4-{[4-({[6-bromo-2-(trifluoromethyl)quinolin-4-yl]carbonyl}amino)-3,5-dimethyl-1H-pyrazol-1-yl]methyl}benzoic acid,
6-bromo-N-[1-(4-carbamoylbenzyl)-3,5-dimethyl-1H-pyrazol-4-yl]-2-(trifluoromethyl)quinoline-4-carboxamide,
6-bromo-N-{3,5-dimethyl-1-[4-(phenylcarbamoyl)benzyl]-1H-pyrazol-4-yl}-2-(trifluoromethyl)quinoline-4-carboxamide,
6-bromo-N-{3,5-dimethyl-1-[4-(methylcarbamoyl)benzyl]-1H-pyrazol-4-yl}-2-(trifluoromethyl)quinoline-4-carboxamide,
6-bromo-N-[3,5-dimethyl-1-(4-{[2-(morpholin-4-yl)ethyl]carbamoyl}benzyl)-1H-pyrazol-4-yl]-2-(trifluoromethyl)quinoline-4-carboxamide,
6-bromo-N-(1-{4-[(2-methoxyethyl)carbamoyl]benzyl}-3,5-dimethyl-1H-pyrazol-4-yl)-2-(trifluoromethyl)quinoline-4-carboxamide,
6-bromo-N-(3,5-dimethyl-1-{4-[(pyridin-3-ylmethyl)carbamoyl]benzyl}-1H-pyrazol-4-yl)-2-(trifluoromethyl)quinoline-4-carboxamide,
6-bromo-N-(1-{4-[(2-hydroxyethyl)carbamoyl]benzyl}-3,5-dimethyl-1H-pyrazol-4-yl)-2-(trifluoromethyl)quinoline-4-carboxamide,
N-{1-[4-(benzylcarbamoyl)benzyl]-3,5-dimethyl-1H-pyrazol-4-yl}-6-bromo-2-(trifluoromethyl)quinoline-4-carboxamide,
6-bromo-N-{3,5-dimethyl-1-[4-(morpholin-4-ylcarbonyl)benzyl]-1H-pyrazol-4-yl}-2-(trifluoromethyl)quinoline-4-carboxamide,
6-bromo-N-[1-(4-{[2-(dimethylamino)ethyl]carbamoyl}benzyl)-3,5-dimethyl-1H-pyrazol-4-yl]-2-(trifluoromethyl)quinoline-4-carboxamide,
6-bromo-N-{1-[4-(dimethylcarbamoyl)benzyl]-3,5-dimethyl-1H-pyrazol-4-yl}-2-(trifluoromethyl)quinoline-4-carboxamide,
6-bromo-2-cyclobutyl-N-[1-(3,4-difluorobenzyl)-3,5-dimethyl-1H-pyrazol-4-yl]quinoline-4-carboxamide,
6-chloro-2-cyclopropyl-N-[1-(3,4-difluorobenzyl)-3,5-dimethyl-1H-pyrazol-4-yl]quinoline-4-carboxamide,
6-bromo-2-cyclobutyl-N-[1-(4-fluorobenzyl)-3,5-dimethyl-1H-pyrazol-4-yl]quinoline-4-carboxamide,
6-bromo-N-{1-[(6-cyanopyridin-3-yl)methyl]-3,5-dimethyl-1H-pyrazol-4-yl}-2-(trifluoromethyl)quinoline-4-carboxamide,
6-bromo-N-[1-(4-chlorobenzyl)-3,5-dimethyl-1H-pyrazol-4-yl]-2-(trifluoromethyl)quinoline-4-carboxamide,
methyl 4-{[1-(4-fluorobenzyl)-3,5-dimethyl-1H-pyrazol-4-yl]carbamoyl}-2-(trifluoromethyl)quinoline-6-carboxylate,
methyl (4-{[4-({[6-bromo-2-(trifluoromethyl)quinolin-4-yl]carbonyl}amino)-3,5-dimethyl-1H-pyrazol-1-yl]methyl}phenyl)acetate,
(4-{[4-({[6-bromo-2-(trifluoromethyl)quinolin-4-yl]carbonyl}amino)-3,5-dimethyl-1H-pyrazol-1-yl]methyl}phenyl)acetic acid,
N-{1-[4-(2-amino-2-oxoethyl)benzyl]-3,5-dimethyl-1H-pyrazol-4-yl}-6-bromo-2-(trifluoromethyl)quinoline-4-carboxamide,
6-bromo-N-(3,5-dimethyl-1-{4-[2-(methylamino)-2-oxoethyl]benzyl}-1H-pyrazol-4-yl)-2-(trifluoromethyl)quinoline-4-carboxamide,
N-(1-{4-[2-(benzylamino)-2-oxoethyl]benzyl}-3,5-dimethyl-1H-pyrazol-4-yl)-6-bromo-2-(trifluoromethyl)quinoline-4-carboxamide,
6-bromo-N-(3,5-dimethyl-1-{4-[2-oxo-2-(phenylamino)ethyl]benzyl}-1H-pyrazol-4-yl)-2-(trifluoromethyl)quinoline-4-carboxamide,
6-bromo-N-(1-{4-[2-(dimethylamino)-2-oxoethyl]benzyl}-3,5-dimethyl-1H-pyrazol-4-yl)-2-(trifluoromethyl)quinoline-4-carboxamide,
6-bromo-N-[3,5-dimethyl-1-(4-{2-oxo-2-[(pyridin-3-ylmethyl)amino]ethyl}benzyl)-1H-pyrazol-4-yl]-2-(trifluoromethyl)quinoline-4-carboxamide,
6-bromo-N-(3,5-dimethyl-1-{4-[2-(morpholin-4-yl)-2-oxoethyl]benzyl}-1H-pyrazol-4-yl)-2-(trifluoromethyl)quinoline-4-carboxamide,
6-bromo-N-[1-(4-{2-[(2-hydroxyethyl)amino]-2-oxoethyl}benzyl)-3,5-dimethyl-1H-pyrazol-4-yl]-2-(trifluoromethyl)quinoline-4-carboxamide,
6-bromo-N-[3,5-dimethyl-1-(pyridin-4-ylmethyl)-1H-pyrazol-4-yl]-2-(trifluoromethyl)quinoline-4-carboxamide,
6-bromo-N-[1-(4-cyanobenzyl)-5-methyl-3-(trifluoromethyl)-1H-pyrazol-4-yl]-2-(trifluoromethyl)quinoline-4-carboxamide,
2-cyclopropyl-N-[1-(3,4-difluorobenzyl)-3,5-dimethyl-1H-pyrazol-4-yl]-6-fluoroquinoline-4-carboxamide,
2-(dimethylamino)-N-[1-(4-fluorobenzyl)-3,5-dimethyl-1H-pyrazol-4-yl]quinoline-4-carboxamide,
6-bromo-N-[1-(4-hydroxybenzyl)-3,5-dimethyl-1H-pyrazol-4-yl]-2-(trifluoromethyl)quinoline-4-carboxamide,
6-chloro-N-[1-(3,4-difluorobenzyl)-3,5-dimethyl-1H-pyrazol-4-yl]-2-(trifluoromethyl)quinoline-4-carboxamide,
N-[1-(4-fluorobenzyl)-3,5-dimethyl-1H-pyrazol-4-yl]-6-phenyl-2-(trifluoromethyl)quinoline-4-carboxamide,
6,8-dichloro-N-[1-(4-cyanobenzyl)-5-methyl-3-(trifluoromethyl)-1H-pyrazol-4-yl]-2-(trifluoromethyl)quinoline-4-carboxamide,
N-[1-(3,4-difluorobenzyl)-3,5-dimethyl-1H-pyrazol-4-yl]-7-fluoro-2-(trifluoromethyl)quinoline-4-carboxamide,
N-[1-(4-cyanobenzyl)-3,5-dimethyl-1H-pyrazol-4-yl]-6,7-difluoro-2-(trifluoromethyl)quinoline-4-carboxamide,
6-bromo-N-[1-(cyclohexylmethyl)-3,5-dimethyl-1H-pyrazol-4-yl]-2-(trifluoromethyl)quinoline-4-carboxamide,
6-bromo-N-[3,5-dimethyl-1-(pyridin-3-ylmethyl)-1H-pyrazol-4-yl]-2-(trifluoromethyl)quinoline-4-carboxamide,
N⁴-[1-(4-fluorobenzyl)-3,5-dimethyl-1H-pyrazol-4-yl]-N²-methylquinoline-2,4-dicarboxamide,
N-[1-(3,4-difluorobenzyl)-3,5-dimethyl-1H-pyrazol-4-yl]-5-fluoro-2-(trifluoromethyl)quinoline-4-carboxamide,
N-[1-(3,4-difluorobenzyl)-3,5-dimethyl-1H-pyrazol-4-yl]-6-fluoro-2-(trifluoromethyl)quinoline-4-carboxamide,
N-[1-(2-cyanobenzyl)-3,5-dimethyl-1H-pyrazol-4-yl]-6,7-difluoro-2-(trifluoromethyl)quinoline-4-carboxamide,
6-bromo-N-ethyl-N-[1-(4-fluorobenzyl)-3,5-dimethyl-1H-pyrazol-4-yl]-2-(trifluoromethyl)quinoline-4-carboxamide,
N-[1-(3,4-difluorobenzyl)-3,5-dimethyl-1H-pyrazol-4-yl]-6,7-difluoro-2-(trifluoromethyl)quinoline-4-carboxamide,
N⁴-[1-(4-fluorobenzyl)-5-methyl-3-(trifluoromethyl)-1H-pyrazol-4-yl]-N2-methylquinoline-2,4-dicarboxamide,
6-bromo-N-[1-(2,4-difluorobenzyl)-5-methyl-3-(trifluoromethyl)-1H-pyrazol-4-yl]-2-(trifluoromethyl)quinoline-4-carboxamide,
6-bromo-N-{1-[(5-chlorothiophen-2-yl)methyl]-3,5-dimethyl-1H-pyrazol-4-yl}-2-(trifluoromethyl)quinoline-4-carboxamide,
N-[1-(4-fluorobenzyl)-3,5-dimethyl-1H-pyrazol-4-yl]-2-(trifluoromethyl)quinoline-4-carboxamide,
N-[1-(4-cyanobenzyl)-5-methyl-3-(trifluoromethyl)-1H-pyrazol-4-yl]-6,7-difluoro-2-(trifluoromethyl)quinoline-4-carboxamide,
N-{1-[(6-cyanopyridin-3-yl)methyl]-3,5-dimethyl-1H-pyrazol-4-yl}-6,7-difluoro-2-(trifluoromethyl)quinoline-4-carboxamide,
6-bromo-N-[1-(4-fluorobenzyl)-3,5-dimethyl-1H-pyrazol-4-yl]quinoline-4-carboxamide,
6-bromo-N-[1-(3,4-difluorobenzyl)-3,5-dimethyl-1H-pyrazol-4-yl]-2-methylquinoline-4-carboxamide,
N-[1-(3-chloro-4-fluorobenzyl)-3,5-dimethyl-1H-pyrazol-4-yl]-2,6-dimethylquinoline-4-carboxamide,
6-bromo-N-{3,5-dimethyl-1-[(1-methyl-1H-pyrazol-3-yl)methyl]-1H-pyrazol-4-yl}-2-(trifluoromethyl)quinoline-4-carboxamide,
N-[1-(4-cyanobenzyl)-5-methyl-3-(trifluoromethyl)-1H-pyrazol-4-yl]-2,6-dimethylquinoline-4-carboxamide,
7-bromo-N-[1-(3,4-difluorobenzyl)-3,5-dimethyl-1H-pyrazol-4-yl]-2-(trifluoromethyl)quinoline-4-carboxamide,
6-bromo-N-[1-(3-chloro-4-fluorobenzyl)-3,5-dimethyl-1H-pyrazol-4-yl]-2-(trifluoromethyl)quinoline-4-carboxamide,
N-[1-(3-chloro-4-fluorobenzyl)-3,5-dimethyl-1H-pyrazol-4-yl]-6,7-difluoro-2-(trifluoromethyl)quinoline-4-carboxamide,
N-[1-(4-fluorobenzyl)-3,5-dimethyl-1H-pyrazol-4-yl]-6-methoxyquinoline-4-carboxamide,
6,7-difluoro-N-[1-(4-fluorobenzyl)-5-methyl-3-(trifluoromethyl)-1H-pyrazol-4-yl]-2-(trifluoromethyl)quinoline-4-carboxamide,
6-bromo-N-[1-(4-fluorobenzyl)-3,5-dimethyl-1H-pyrazol-4-yl]-N-(2-methoxyethyl)-2-(trifluoromethyl)quinoline-4-carboxamide,
N-[1-(cyclohexylmethyl)-3,5-dimethyl-1H-pyrazol-4-yl]-6,7-difluoro-2-(trifluoromethyl)quinoline-4-carboxamide,
N-[3,5-dimethyl-1-(pyridin-3-ylmethyl)-1H-pyrazol-4-yl]-6,7-difluoro-2-(trifluoromethyl)quinoline-4-carboxamide,
6-bromo-N-[1-(3,4-difluorobenzyl)-5-methyl-3-(trifluoromethyl)-1H-pyrazol-4-yl]-2-(trifluoromethyl)quinoline-4-carboxamide,
N-[1-(4-fluorobenzyl)-3,5-dimethyl-1H-pyrazol-4-yl]-2-(morpholin-4-yl)quinoline-4-carboxamide,
6,8-dichloro-N-[1-(3,4-difluorobenzyl)-5-methyl-3-(trifluoromethyl)-1H-pyrazol-4-yl]-2-(trifluoromethyl)quinoline-4-carboxamide,
N-benzyl-6-bromo-N-[1-(4-fluorobenzyl)-3,5-dimethyl-1H-pyrazol-4-yl]-2-(trifluoromethyl)quinoline-4-carboxamide,
N-{1-[(6-cyanopyridin-3-yl)methyl]-3,5-dimethyl-1H-pyrazol-4-yl}-2,6-dimethylquinoline-4-carboxamide,
N-(1-{4-[(2-hydroxyethyl)carbamoyl]benzyl}-3,5-dimethyl-1H-pyrazol-4-yl)-2, 6-dimethylquinoline-4-carboxamide,
N-[1-(3,4-difluorobenzyl)-5-methyl-3-(trifluoromethyl)-1H-pyrazol-4-yl]-2,6-dimethylquinoline-4-carboxamide,
N-[1-(2,4-difluorobenzyl)-5-methyl-3-(trifluoromethyl)-1H-pyrazol-4-yl]-2,6-dimethylquinoline-4-carboxamide,
6,8-dichloro-N-[1-(2,4-difluorobenzyl)-5-methyl-3-(trifluoromethyl)-1H-pyrazol-4-yl]-2-(trifluoromethyl)quinoline-4-carboxamide
N-[1-(cyclohexylmethyl)-3,5-dimethyl-1H-pyrazol-4-yl]-2,6-dimethylquinoline-4-carboxamide,
N-[1-(3,4-difluorobenzyl)-5-methyl-3-(trifluoromethyl)-1H-pyrazol-4-yl]-6,7-difluoro-2-(trifluoromethyl)quinoline-4-carboxamide,
N-[3,5-dimethyl-1-(pyridin-3-ylmethyl)-1H-pyrazol-4-yl]-2,6-dimethylquinoline-4-carboxamide,
N⁴-[1-(4-fluorobenzyl)-3,5-dimethyl-1H-pyrazol-4-yl]-N²,N²-dimethylquinoline-2,4-dicarboxamide,
N-[1-(2,4-difluorobenzyl)-5-methyl-3-(trifluoromethyl)-1H-pyrazol-4-yl]-6,7-difluoro-2-(trifluoromethyl)quinoline-4-carboxamide,
N-[1-(4-fluorobenzyl)-3,5-dimethyl-1H-pyrazol-4-yl]quinoline-4-carboxamide,
6-chloro-N⁴-[1-(4-cyanobenzyl)-3,5-dimethyl-1H-pyrazol-4-yl]-7-fluoroquinoline-2,4-dicarboxamide,
6-chloro-N⁴-[1-(4-cyanobenzyl)-5-methyl-3-(trifluoromethyl)-1H-pyrazol-4-yl]-7-fluoroquinoline-2,4-dicarboxamide,
6-chloro-7-fluoro-N⁴-[1-(4-methoxybenzyl)-3,5-dimethyl-1H-pyrazol-4-yl]quinoline-2,4-dicarboxamide,
6-chloro-N⁴-[1-(2-cyanobenzyl)-3,5-dimethyl-1H H-pyrazol-4-yl]-7-fluoroquinoline-2,4-dicarboxamide,
N⁴-[1-(4-cyanobenzyl)-5-methyl-3-(trifluoromethyl)-1H-pyrazol-4-yl]-6,7-difluoroquinoline-2,4-dicarboxamide,
6-bromo-N-{3,5-dimethyl-1-[4-(methylsulfonyl)benzyl]-1H-pyrazol-4-yl}-2-(trifluoromethyl)quinoline-4-carboxamide,
6-bromo-N-[1-(4-cyano-3-fluorobenzyl)-3,5-dimethyl-1H-pyrazol-4-yl]-2-(trifluoromethyl)quinoline-4-carboxamide,
N⁴-[1-(4-cyanobenzyl)-5-methyl-3-(trifluoromethyl)-1H-pyrazol-4-yl]-6-fluoroquinoline-2,4-dicarboxamide,
N⁴-{1-[(2-cyanopyridin-3-yl)methyl]-3,5-dimethyl-1H-pyrazol-4-yl}-6,7-difluoroquinoline-2,4-dicarboxamide,
N⁴-[1-(2-cyanobenzyl)-3,5-dimethyl-1H-pyrazol-4-yl]-6-fluoroquinoline-2,4-dicarboxamide,
N⁴-{1-[(3-cyanopyridin-2-yl)methyl]-3,5-dimethyl-1H-pyrazol-4-yl}-6,7-difluoroquinoline-2,4-dicarboxamide,
N⁴-{1-[(2-cyanopyridin-3-yl)methyl]-3,5-dimethyl-1H-pyrazol-4-yl}quinoline-2,4-dicarboxamide,
N⁴-{3,5-dimethyl-1-[4-(methylsulfonyl)benzyl]-1H-pyrazol-4-yl}quinoline-2,4-dicarboxamide,
N⁴-{1-[(6-cyanopyridin-3-yl)methyl]-3,5-dimethyl-1H-pyrazol-4-yl}quinoline-2,4-dicarboxamide,
6,7-difluoro-N⁴-[1-(4-methoxybenzyl)-3,5-dimethyl-1H-pyrazol-4-yl]quinoline-2,4-dicarboxamide,
6-fluoro-N⁴-[1-(4-methoxybenzyl)-3,5-dimethyl-1H-pyrazol-4-yl]quinoline-2,4-dicarboxamide,
N⁴-[1-(4-cyanobenzyl)-3,5-dimethyl-1H-pyrazol-4-yl]-6-fluoroquinoline-2,4-dicarboxamide,
6-fluoro-N⁴-[1-(4-fluorobenzyl)-3,5-dimethyl-1H-pyrazol-4-yl]quinoline-2,4-dicarboxamide,
N⁴-[1-(4-cyanobenzyl)-3,5-dimethyl-1H-pyrazol-4-yl]-6,7-difluoroquinoline-2,4-dicarboxamide,
N⁴-{3,5-dimethyl-1-[4-(methylsulfonyl)benzyl]-1H-pyrazol-4-yl}-6,7-difluoroquinoline-2,4-dicarboxamide,
N⁴-[1-(2-cyanobenzyl)-3,5-dimethyl-1H-pyrazol-4-yl]quinoline-2,4-dicarboxamide,
2-cyclopropyl-N-[1-(4-fluorobenzyl)-3,5-dimethyl-1H-pyrazol-4-yl]-6-methoxyquinoline-4-carboxamide,
6-bromo-N-{1-[(3-cyanopyridin-2-yl)methyl]-3,5-dimethyl-1H-pyrazol-4-yl}-2-(trifluoromethyl)quinoline-4-carboxamide,
N⁴-[1-(2-cyanobenzyl)-3,5-dimethyl-1H-pyrazol-4-yl]-6,7-difluoroquinoline-2,4-dicarboxamide,
6,7-difluoro-N⁴-[1-(3-fluoro-4-methoxybenzyl)-3,5-dimethyl-1H-pyrazol-4-yl]quinoline-2,4-dicarboxamide,
6-chloro-7-fluoro-N⁴-[1-(3-fluoro-4-methoxybenzyl)-3,5-dimethyl-1H-pyrazol-4-yl]quinoline-2,4-dicarboxamide,
N⁴-[1-(3-fluoro-4-methoxybenzyl)-3,5-dimethyl-1H-pyrazol-4-yl]quinoline-2,4-dicarboxamide,
N-[1-(3-fluoro-4-methoxybenzyl)-3,5-dimethyl-1H-pyrazol-4-yl]-2-methoxyquinoline-4-carboxamide,
N⁴-[1-(4-cyanobenzyl)-5-methyl-3-(trifluoromethyl)-1H-pyrazol-4-yl]-7-fluoroquinoline-2,4-dicarboxamide,
6-chloro-N⁴-[1-(4-cyanobenzyl)-5-methyl-3-(trifluoromethyl)-1H-pyrazol-4-yl]-8-fluoroquinoline-2,4-dicarboxamide,
N⁴-[1-(4-cyanobenzyl)-5-methyl-3-(trifluoromethyl)-1H-pyrazol-4-yl]-7-fluoro-6-methoxyquinoline-2,4-dicarboxamide,
N⁴-[1-(4-cyanobenzyl)-5-methyl-3-(trifluoromethyl)-1H-pyrazol-4-yl]-5,7-difluoroquinoline-2,4-dicarboxamide,
N⁴-[1-(4-cyanobenzyl)-5-methyl-3-(trifluoromethyl)-1H-pyrazol-4-yl]-5-methylquinoline-2,4-dicarboxamide,
N⁴-[1-(4-cyanobenzyl)-5-methyl-3-(trifluoromethyl)-1H-pyrazol-4-yl]-6-methoxyquinoline-2,4-dicarboxamide,
7-chloro-N⁴-[1-(4-cyanobenzyl)-5-methyl-3-(trifluoromethyl)-1H-pyrazol-4-yl]quinoline-2,4-dicarboxamide,
N⁴-[1-(4-cyanobenzyl)-5-methyl-3-(trifluoromethyl)-1H-pyrazol-4-yl]-8-fluoroquinoline-2,4-dicarboxamide,
6-chloro-N⁴-[1-(4-cyanobenzyl)-5-methyl-3-(trifluoromethyl)-1H-pyrazol-4-yl]quinoline-2,4-dicarboxamide,
N⁴-[1-(4-cyanobenzyl)-5-methyl-3-(trifluoromethyl)-1H-pyrazol-4-yl]-5-fluoroquinoline-2,4-dicarboxamide,
N⁴-[1-(4-cyanobenzyl)-5-methyl-3-(trifluoromethyl)-1H-pyrazol-4-yl]-5-fluoroquinoline-2,4-dicarboxamide,
N⁴-[1-(4-cyanobenzyl)-3,5-dimethyl-1H H-pyrazol-4-yl]-7-fluoroquinoline-2,4-dicarboxamide,
6-chloro-N⁴-[1-(4-cyanobenzyl)-3,5-dimethyl-1H-pyrazol-4-yl]-8-fluoroquinoline-2,4-dicarboxamide,
N⁴-[1-(4-cyanobenzyl)-3,5-dimethyl-1H-pyrazol-4-yl]-5,7-difluoroquinoline-2,4-dicarboxamide,
N⁴-[1-(4-cyanobenzyl)-3,5-dimethyl-1H-pyrazol-4-yl]-7-fluoro-6-methoxyquinoline-2,4-dicarboxamide,
8-chloro-N⁴-[1-(4-cyanobenzyl)-3,5-dimethyl-1H-pyrazol-4-yl]quinoline-2,4-dicarboxamide,
N⁴-[1-(4-cyanobenzyl)-3,5-dimethyl-1H-pyrazol-4-yl]-7-fluoro-6-methylquinoline-2,4-dicarboxamide,
N⁴-[1-(4-cyanobenzyl)-3,5-dimethyl-1H-pyrazol-4-yl]-5-methylquinoline-2,4-dicarboxamide,
N⁴-[1-(4-cyanobenzyl)-3,5-dimethyl-1H-pyrazol-4-yl]-6-methoxyquinoline-2,4-dicarboxamide,
N⁴-[1-(4-cyanobenzyl)-3,5-dimethyl-1H-pyrazol-4-yl]quinoline-2,4-dicarboxamide,
7-chloro-N⁴-[1-(4-cyanobenzyl)-3,5-dimethyl-1H-pyrazol-4-yl]quinoline-2,4-dicarboxamide,
6-chloro-N⁴-[1-(4-cyanobenzyl)-3,5-dimethyl-1H-pyrazol-4-yl]quinoline-2,4-dicarboxamide,
N⁴-[1-(4-cyanobenzyl)-3,5-dimethyl-1H-pyrazol-4-yl]-5-fluoroquinoline-2,4-dicarboxamide,
N⁴-[1-(4-cyanobenzyl)-3,5-dimethyl-1H-pyrazol-4-yl]-8-fluoroquinoline-2,4-dicarboxamide,
N⁴-[1-(4-cyanobenzyl)-3,5-dimethyl-1H-pyrazol-4-yl]-6-methylquinoline-2,4-dicarboxamide,
N-[1-(4-cyanobenzyl)-3,5-dimethyl-1H-pyrazol-4-yl]-6-fluoro-2-methoxyquinoline-4-carboxamide,
N⁴-[1-(2-cyanobenzyl)-3,5-dimethyl-1H-pyrazol-4-yl]-7-fluoroquinoline-2,4-dicarboxamide,
N⁴-[1-(2-cyanobenzyl)-3,5-dimethyl-1H-pyrazol-4-yl]-5-fluoroquinoline-2,4-dicarboxamide,
6-bromo-N⁴-[1-(2-cyanobenzyl)-3,5-dimethyl-1H-pyrazol-4-yl]quinoline-2,4-dicarboxamide,
6-chloro-N⁴-[1-(2-cyanobenzyl)-3,5-dimethyl-1H-pyrazol-4-yl]-8-fluoroquinoline-2,4-dicarboxamide,
6-chloro-N⁴-[1-(2-cyanobenzyl)-5-methyl-3-(trifluoromethyl)-1H-pyrazol-4-yl]-7-fluoroquinoline-2,4-dicarboxamide,
N⁴-[1-(2-cyanobenzyl)-5-methyl-3-(trifluoromethyl)-1H-pyrazol-4-yl]quinoline-2,4-dicarboxamide,
N⁴-[1-(2-cyanobenzyl)-5-methyl-3-(trifluoromethyl)-1H-pyrazol-4-yl]-7-fluoroquinoline-2,4-dicarboxamide,
N⁴-[1-(2-cyanobenzyl)-5-methyl-3-(trifluoromethyl)-1H-pyrazol-4-yl]-5-fluoroquinoline-2,4-dicarboxamide,
6-bromo-N⁴-[1-(2-cyanobenzyl)-5-methyl-3-(trifluoromethyl)-1H-pyrazol-4-yl]quinoline-2,4-dicarboxamide,
7-fluoro-N⁴-[1-(4-methoxybenzyl)-3,5-dimethyl-1H-pyrazol-4-yl]quinoline-2,4-dicarboxamide,
6-chloro-8-fluoro-N⁴-[1-(4-methoxybenzyl)-3,5-dimethyl-1H-pyrazol-4-yl]quinoline-2,4-dicarboxamide,
N⁴-[1-(4-methoxybenzyl)-5-methyl-3-(trifluoromethyl)-1H-pyrazol-4-yl]quinoline-2,4-dicarboxamide,
6-chloro-7-fluoro-N⁴-[1-(4-methoxybenzyl)-5-methyl-3-(trifluoromethyl)-1H-pyrazol-4-yl]quinoline-2,4-dicarboxamide,
6,7-difluoro-N⁴-[1-(4-methoxybenzyl)-5-methyl-3-(trifluoromethyl)-1H-pyrazol-4-yl]quinoline-2,4-dicarboxamide,
N⁴-{[(6-methoxypyridin-3-yl)methyl]-5-methyl-3-(trifluoromethyl)-1H-pyrazol-4-yl}quinoline-2,4-dicarboxamide,
6-chloro-7-fluoro-N⁴-{1-[(6-methoxypyridin-3-yl)methyl]-5-methyl-3-(trifluoromethyl)-1H-pyrazol-4-yl}quinoline-2,4-dicarboxamide,
N⁴-{1[(6-methoxypyridin-3-yl)methyl]-3,5-dimethyl-1H-pyrazol-4-yl}quinoline-2,4-dicarboxamide,
6-chloro-7-fluoro-N⁴-{1-[(6-methoxypyridin-3-yl)methyl]-3,5-dimethyl-1H-pyrazol-4-yl}quinoline-2,4-dicarboxamide,
N⁴-{1-[3-(4-methoxyphenyl)propyl]-3,5-dimethyl-1H-pyrazol-4-yl}quinoline-2,4-dicarboxamide,
6-chloro-N⁴-{1-[(3-cyanopyridin-4-yl)methyl]-3,5-dimethyl-1H-pyrazol-4-yl}-7-fluoroquinoline-2,4-dicarboxamide,
N⁴-{1-[(3-cyanopyridin-4-yl)methyl]-3,5-dimethyl-1H-pyrazol-4-yl}quinoline-2,4-dicarboxamide,
6-chloro-N⁴-{1-[(3-cyanopyridin-4-yl)methyl]-5-methyl-3-(trifluoromethyl)-1H-pyrazol-4-yl}-7-fluoroquinoline-2,4-dicarboxamide,
N⁴-{1[(3-cyanopyridin-4-yl)methyl]-5-methyl-3-(trifluoromethyl)-1H-pyrazol-4-yl}quinoline-2,4-dicarboxamide,
N⁴-{[(6-cyanopyridin-3-yl)methyl]-5-methyl-3-(trifluoromethyl)-1H-pyrazol-4-yl}quinoline-2,4-dicarboxamide,
N⁴-{[(6-cyanopyridin-3-yl)methyl]-5-methyl-3-(trifluoromethyl)-1H-pyrazol-4-yl}-7-fluoroquinoline-2,4-dicarboxamide,
N⁴-{[(6-cyanopyridin-3-yl)methyl]-5-methyl-3-(trifluoromethyl)-1H-pyrazol-4-yl}-5-fluoroquinoline-2,4-dicarboxamide,
N⁴-{1-[(6-cyanopyridin-3-yl)methyl]-3,5-dimethyl-1H-pyrazol-4-yl}-7-fluoroquinoline-2,4-dicarboxamide,
6-bromo-N⁴-{1-[(6-cyanopyridin-3-yl)methyl]-3,5-dimethyl-1H-pyrazol-4-yl}quinoline-2,4-dicarboxamide,
6-chloro-N⁴-{1-[2-(4-cyanophenoxy)ethyl]-3,5-dimethyl-1H-pyrazol-4-yl}-7-fluoroquinoline-2,4-dicarboxamide,
N⁴-{1-[2-(4-cyanophenoxy)ethyl]-3,5-dimethyl-1H-pyrazol-4-yl}quinoline-2,4-dicarboxamide,
6-chloro-N⁴-[1-(4-cyano-2-fluorobenzyl)-3,5-dimethyl-1H-pyrazol-4-yl]-7-fluoroquinoline-2,4-dicarboxamide,
N⁴-[1-(4-cyano-2-fluorobenzyl)-3,5-dimethyl-1H-pyrazol-4-yl]quinoline-2,4-dicarboxamide,
N⁴-[1-(4-cyano-2-fluorobenzyl)-3,5-dimethyl-1H-pyrazol-4-yl]-6,7-difluoroquinoline-2,4-dicarboxamide,
6-bromo-N⁴-[1-(4-cyano-2-fluorobenzyl)-3,5-dimethyl-1H-pyrazol-4-yl]quinoline-2,4-dicarboxamide,
N⁴-[1-(4-cyano-2-fluorobenzyl)-3,5-dimethyl-1H-pyrazol-4-yl]-7-fluoroquinoline-2,4-dicarboxamide,
N⁴-[1-(4-cyano-2-fluorobenzyl)-3,5-dimethyl-1H-pyrazol-4-yl]-5-fluoroquinoline-2,4-dicarboxamide,
N⁴-[1-(4-cyano-2-fluorobenzyl)-5-methyl-3-(trifluoromethyl)-1H-pyrazol-4-yl]quinoline-2,4-dicarboxamide,
6-chloro-N⁴-[1-(4-cyano-2-fluorobenzyl)-5-methyl-3-(trifluoromethyl)-1H-pyrazol-4-yl]-7-fluoroquinoline-2,4-dicarboxamide,
N⁴-[1-(4-cyano-2-fluorobenzyl)-5-methyl-3-(trifluoromethyl)-1H-pyrazol-4-yl]-6,7-difluoroquinoline-2,4-dicarboxamide,
6-bromo-N⁴-[1-(4-cyano-2-fluorobenzyl)-5-methyl-3-(trifluoromethyl)-1H-pyrazol-4-yl]quinoline-2,4-dicarboxamide,
N⁴-[1-(4-cyano-2-fluorobenzyl)-5-methyl-3-(trifluoromethyl)-1H-pyrazol-4-yl]-5-fluoroquinoline-2,4-dicarboxamide,
6-chloro-N⁴-[3-cyano-1-(4-fluorobenzyl)-5-methyl-1H-pyrazol-4-yl]-7-fluoroquinoline-2,4-dicarboxamide,
N⁴-[3-cyano-1-(4-fluorobenzyl)-5-methyl-1H-pyrazol-4-yl]quinoline-2,4-dicarboxamide,
6-bromo-N-[1-(4-cyanobenzyl)-3,5-diethyl-1H-pyrazol-4-yl]-2-(trifluoromethyl)quinoline-4-carboxamide,
N⁴-[1-(4-cyanobenzyl)-3,5-diethyl-1H-pyrazol-4-yl]quinoline-2,4-dicarboxamide,
6-chloro-N⁴-[1-(4-cyanobenzyl)-3,5-diethyl-1H-pyrazol-4-yl]-7-fluoroquinoline-2,4-dicarboxamide,
N⁴-[1-(4-cyanobenzyl)-3,5-diethyl-1H-pyrazol-4-yl]-7-fluoroquinoline-2,4-dicarboxamide,
7-chloro-N⁴-[1-(4-cyanobenzyl)-3,5-diethyl-1H-pyrazol-4-yl]quinoline-2,4-dicarboxamide,
6-chloro-N⁴-[1-(4-cyanobenzyl)-5-ethyl-3-(trifluoromethyl)-1H-pyrazol-4-yl]-7-fluoroquinoline-2,4-dicarboxamide,
N⁴-[1-(4-cyanobenzyl)-5-ethyl-3-(trifluoromethyl)-1H-pyrazol-4-yl]-7-fluoroquinoline-2,4-dicarboxamide,
N⁴-[1-(4-cyanobenzyl)-5-ethyl-3-(trifluoromethyl)-1H-pyrazol-4-yl]-5-fluoroquinoline-2,4-dicarboxamide,
N⁴-[1-(4-cyanobenzyl)-5-isopropyl-3-(trifluoromethyl)-1H-pyrazol-4-yl]quinoline-2,4-dicarboxamide,
N⁴-[1-(4-cyanobenzyl)-5-isopropyl-3-(trifluoromethyl)-1H-pyrazol-4-yl]-7-fluoroquinoline-2,4-dicarboxamide,
N⁴-[1-(4-cyanobenzyl)-3-isopropyl-5-(trifluoromethyl)-1H-pyrazol-4-yl]quinoline-2,4-dicarboxamide,
N⁴-[1-(4-cyanobenzyl)-3-isopropyl-5-(trifluoromethyl)-1H-pyrazol-4-yl]-7-fluoroquinoline-2,4-dicarboxamide,
N⁴-[1-(4-cyanobenzyl)-3-isopropyl-5-methyl-1H-pyrazol-4-yl]-7-fluoroquinoline-2,4-dicarboxamide,
N⁴-[1-(4-cyanobenzyl)-3-isopropyl-5-methyl-1H-pyrazol-4-yl]quinoline-2,4-dicarboxamide,
N⁴-[1-(4-cyanobenzyl)-5-isopropyl-3-methyl-1H-pyrazol-4-yl]quinoline-2,4-dicarboxamide,
6-chloro-N⁴-[1-(4-cyanobenzyl)-3-isopropyl-5-methyl-1H-pyrazol-4-yl]-7-fluoroquinoline-2,4-dicarboxamide,
N⁴-[1-(4-cyanobenzyl)-3-ethyl-5-methyl-1H-pyrazol-4-yl]quinoline-2,4-dicarboxamide,
N⁴-[1-(4-cyanobenzyl)-5-ethyl-3-methyl-1H-pyrazol-4-yl]quinoline-2,4-dicarboxamide,
N⁴-[1-(4-cyanobenzyl)-3-ethyl-5-methyl-1H-pyrazol-4-yl]-7-fluoroquinoline-2,4-dicarboxamide,
N⁴-[1-(4-cyanobenzyl)-5-ethyl-3-methyl-1H-pyrazol-4-yl]-7-fluoroquinoline-2,4-dicarboxamide,
(±)-N⁴-{1-[1-(4-cyanophenyl)ethyl]-3,5-dimethyl-1H-pyrazol-4-yl}quinoline-2,4-dicarboxamide,
(±)-6-chloro-N⁴-{1-[1-(4-cyanophenyl)ethyl]-3,5-dimethyl-1H-pyrazol-4-yl}-7-fluoroquinoline-2,4-dicarboxamide,
(±)-N⁴-{1-[1-(4-cyanophenyl)ethyl]-3,5-dimethyl-1H-pyrazol-4-yl}-8-fluoroquinoline-2,4-dicarboxamide,
(±)-N⁴-{1-[1-(4-cyanophenyl)ethyl]-3,5-dimethyl-1H-pyrazol-4-yl}-8-fluoroquinoline-2,4-dicarboxamide,
(±)-N⁴-{1-[1-(4-cyanophenyl)ethyl]-5-methyl-3-(trifluoromethyl)-1H-pyrazol-4-yl}quinoline-2,4-dicarboxamide,
(±)-7-fluoro-N⁴-[5-methyl-1-(1-phenylethyl)-3-(trifluoromethyl)-1H-pyrazol-4-yl]quinoline-2,4-dicarboxamide,
(R)-7-fluoro-N⁴-[5-methyl-1-(1-phenylethyl)-3-(trifluoromethyl)-1H-pyrazol-4-yl]quinoline-2,4-dicarboxamide,
(S)-7-fluoro-N⁴-[5-methyl-1-(1-phenylethyl)-3-(trifluoromethyl)-1H-pyrazol-4-yl]quinoline-2,4-dicarboxamide,
6-cyano N⁴-[1-(4-cyanobenzyl)-5-methyl-3-(trifluoromethyl)-1H-pyrazol-4-yl]quinoline-2,4-dicarboxamide,
8-chloro-N⁴-[1-(4-cyanobenzyl)-5-methyl-3-(trifluoromethyl)-1H-pyrazol-4-yl]quinoline-2,4-dicarboxamide,
N⁴-[1-(4-cyanobenzyl)-5-methyl-3-(trifluoromethyl)-1H-pyrazol-4-yl]-7-methoxyquinoline-2,4-dicarboxamide,
2-(azetidin-1-ylcarbonyl)-N-[1-(4-cyanobenzyl)-5-methyl-3-(trifluoromethyl)-1H-pyrazol-4-yl]-7-fluoroquinoline-4-carboxamide,
N⁴-[1-(4-cyanobenzyl)-5-methyl-3-(trifluoromethyl)-1H-pyrazol-4-yl]-7-fluoro-N²-(3-hydroxypropyl)quinoline-2,4-dicarboxamide,
N⁴-[1-(4-cyanobenzyl)-5-methyl-3-(trifluoromethyl)-1H-pyrazol-4-yl]-7-fluoro-N²-[2-(morpholin-4-yl)ethyl]quinoline-2,4-dicarboxamide,
N⁴-[1-(4-cyanobenzyl)-5-methyl-3-(trifluoromethyl)-1H-pyrazol-4-yl]-7-fluoro-6-methylquinoline-2,4-dicarboxamide,
N⁴-[1-(4-cyanobenzyl)-5-methyl-3-(trifluoromethyl)-1H-pyrazol-4-yl]-6-[(2-methoxyethyl)amino]quinoline-2,4-dicarboxamide,
N⁴-[1-(4-cyanobenzyl)-5-methyl-3-(trifluoromethyl)-1H-pyrazol-4-yl]-6-(piperidin-1-yl)quinoline-2,4-dicarboxamide,
6-chloro-N⁴-{1-[3-(4-cyanophenyl)propyl]-3,5-dimethyl-1H-pyrazol-4-yl}-7-fluoroquinoline-2,4-dicarboxamide,
N⁴-{1-[3-(4-cyanophenyl)propyl]-3,5-dimethyl-1H-pyrazol-4-yl}quinoline-2,4-dicarboxamide,
6-bromo-N-{1-[3-(4-cyanophenyl)propyl]-3,5-dimethyl-1H-pyrazol-4-yl}-2-(trifluoromethyl)quinoline-4-carboxamide,
N-{1-[3-(4-cyanophenyl)propyl]-3,5-dimethyl-1H-pyrazol-4-yl}-2-methoxyquinoline-4-carboxamide,
6-bromo-N⁴-{1-[(5-cyanopyridin-2-yl)methyl]-3,5-dimethyl-1H-pyrazol-4-yl}quinoline-2,4-dicarboxamide,
6-chloro-N⁴-{1-[(5-cyanopyridin-2-yl)methyl]-3,5-dimethyl-1H-pyrazol-4-yl}-7-fluoroquinoline-2,4-dicarboxamide,
N-{1[(5-cyanopyridin-2-yl)methyl]-3,5-dimethyl-1H-pyrazol-4-yl}-5-fluoroquinoline-2,4-dicarboxamide,
6-bromo-N-{1-[(5-cyanopyridin-2-yl)methyl]-3,5-dimethyl-1H-pyrazol-4-yl}-2-(trifluoromethyl)quinoline-4-carboxamide,
N⁴{-[(5-cyanopyridin-2-yl)methyl]-3,5-dimethyl-1H-pyrazol-4-yl}-6,7-difluoroquinoline-2,4-dicarboxamide,
N⁴{-[(5-cyanopyridin-2-yl)methyl]-3,5-dimethyl-1H-pyrazol-4-yl}quinoline-2,4-dicarboxamide,
N⁴{-[(5-cyanopyridin-2-yl)methyl]-3,5-dimethyl-1H-pyrazol-4-yl}-7-fluoroquinoline-2,4-dicarboxamide,
N-{1-[(5-cyanopyridin-2-yl)methyl]-3,5-dimethyl-1H-pyrazol-4-yl}-2-methoxyquinoline-4-carboxamide,
6-bromo-N⁴-{1-[(5-cyanopyridin-2-yl)methyl]-5-methyl-3-(trifluoromethyl)-1H-pyrazol-4-yl}quinoline-2,4-dicarboxamide,
6-chloro-N⁴-{1-[(5-cyanopyridin-2-yl)methyl]-5-methyl-3-(trifluoromethyl)-1H-pyrazol-4-yl}-7-fluoroquinoline-2,4-dicarboxamide,
N⁴-{[(5-cyanopyridin-2-yl)methyl]-5-methyl-3-(trifluoromethyl)-1H-pyrazol-4-yl}-7-fluoroquinoline-2,4-dicarboxamide,
N⁴-{[(5-cyanopyridin-2-yl)methyl]-5-methyl-3-(trifluoromethyl)-1H-pyrazol-4-yl}quinoline-2,4-dicarboxamide,
6-bromo-N-{1-[(5-cyanopyridin-2-yl)methyl]-5-methyl-3-(trifluoromethyl)-1H-pyrazol-4-yl}-2-(trifluoromethyl)quinoline-4-carboxamide,
N⁴-{[(5-cyanopyridin-2-yl)methyl]-5-methyl-3-(trifluoromethyl)-1H-pyrazol-4-yl}-5-fluoroquinoline-2,4-dicarboxamide,
N⁴-{[(5-cyanopyridin-2-yl)methyl]-5-methyl-3-(trifluoromethyl)-1H-pyrazol-4-yl}-6,7-difluoroquinoline-2,4-dicarboxamide,
N-{1-[(5-cyanopyridin-2-yl)methyl]-5-methyl-3-(trifluoromethyl)-1H-pyrazol-4-yl}-2-methoxyquinoline-4-carboxamide,
N⁴-[1-(4-cyanobenzyl)-5-methyl-3-(trifluoromethyl)-1H-pyrazol-4-yl]-6-methylquinoline-2,4-dicarboxamide,
methyl 4-{[4-{[(2-carbamoyl-7-fluoroquinolin-4-yl)carbonyl]amino}-5-methyl-3-(trifluoromethyl)-1H-pyrazol-1-yl]methyl}benzoate,
4-{[4-{[(2-carbamoyl-7-fluoroquinolin-4-yl)carbonyl]amino}-5-methyl-3-(trifluoromethyl)-1H-pyrazol-1-yl]methyl}benzoic acid,
N⁴-[1-(4-carbamoylbenzyl)-5-methyl-3-(trifluoromethyl)-1H-pyrazol-4-yl]-7-fluoroquinoline-2,4-dicarboxamide,
7-fluoro-N⁴-{5-methyl-1-[4-(methylcarbamoyl)benzyl]-3-(trifluoromethyl)-1H-pyrazol-4-yl}quinoline-2,4-dicarboxamide,
N⁴-{[4-(dimethylcarbamoyl)benzyl]-5-methyl-3-(trifluoromethyl)-1H-pyrazol-4-yl}-7-fluoroquinoline-2,4-dicarboxamide,
N⁴{1-[4-(azetidin-1-ylcarbonyl)benzyl]-5-methyl-3-(trifluoromethyl)-1H-pyrazol-4-yl}-7-fluoroquinoline-2,4-dicarboxamide,
7-fluoro-N⁴-[1-{4-[(2-methoxyethyl)carbamoyl]benzyl}-5-methyl-3-(trifluoromethyl)-1H-pyrazol-4-yl]quinoline-2,4-dicarboxamide,
N⁴-[1-(4-{[2-(dimethylamino)ethyl]carbamoyl}benzyl)-5-methyl-3-(trifluoromethyl)-1H-pyrazol-4-yl]-7-fluoroquinoline-2,4-dicarboxamide,
7-fluoro-N⁴-[1-{4-[(2-hydroxyethyl)carbamoyl]benzyl}-5-methyl-3-(trifluoromethyl)-1H-pyrazol-4-yl]quinoline-2,4-dicarboxamide,
(±)-ethyl [{4-[(4-{[(2-carbamoylquinolin-4-yl)carbonyl]amino}-3,5-dimethyl-1H-pyrazol-1-yl)methyl]phenyl}(methyl)oxido-λ⁶-sulfanylidene]carbamate,
(±)-N⁴-{3,5-dimethyl-1-[4-(S-methylsulfonimidoyl)benzyl]-1H-pyrazol-4-yl}quinoline-2,4-dicarboxamide,
(S)-7-fluoro-N⁴-[5-methyl-1-(1-phenylethyl)-3-(trifluoromethyl)-1H-pyrazol-4-yl]quinoline-2,4-dicarboxamide,
(R)-7-fluoro-N⁴-[5-methyl-1-(1-phenylethyl)-3-(trifluoromethyl)-1H-pyrazol-4-yl]quinoline-2,4-dicarboxamide,
2-bromo-N-[1-(4-cyanobenzyl)-3,5-dimethyl-1H-pyrazol-4-yl]quinoline-4-carboxamide,
N-[1-(4-cyanobenzyl)-3,5-dimethyl-1H-pyrazol-4-yl]-2-(methylsulfanyl)quinoline-4-carboxamide,
N-[1-(4-cyanobenzyl)-5-methyl-3-(trifluoromethyl)-1H-pyrazol-4-yl]-2-(methylsulfanyl)quinoline-4-carboxamide,
(±)-N-[1-(4-cyanobenzyl)-5-methyl-3-(trifluoromethyl)-1H-pyrazol-4-yl]-2-(methylsulfinyl)quinoline-4-carboxamide,
N-[1-(4-cyanobenzyl)-5-methyl-3-(trifluoromethyl)-1H-pyrazol-4-yl]-2-(methylsulfonyl)quinoline-4-carboxamide,
(±)-N-[1-(4-cyanobenzyl)-5-methyl-3-(trifluoromethyl)-1H-pyrazol-4-yl]-2-(N-cyano-S-methylsulfinimidoyl)quinoline-4-carboxamide,
(±)-N-[1-(4-cyanobenzyl)-5-methyl-3-(trifluoromethyl)-1H-pyrazol-4-yl]-2-(N-cyano-S-methylsulfonimidoyl)quinoline-4-carboxamide,
(±)-N-[1-(4-cyanobenzyl)-5-methyl-3-(trifluoromethyl)-1H-pyrazol-4-yl]-2-(S-methylsulfonimidoyl)quinoline-4-carboxamide,
N⁴-(1-{4-[(dimethylamino)methyl]benzyl}-3,5-dimethyl-1H-pyrazol-4-yl)-7-fluoroquinoline-2,4-dicarboxamide,
N⁴-[1-(4-cyanobenzyl)-3-methyl-5-(trifluoromethyl)-1H-pyrazol-4-yl]-7-fluoroquinoline-2,4-dicarboxamide,
N⁴-[1-(4-cyano-2,6-difluorobenzyl)-3,5-dimethyl-1H-pyrazol-4-yl]-7-fluoroquinoline-2,4-dicarboxamide,
N⁴-[1-(4-cyano-2,6-difluorobenzyl)-3,5-dimethyl-1H-pyrazol-4-yl]quinoline-2,4-dicarboxamide,
6-chloro-N⁴-[1-(4-cyano-2, 6-difluorobenzyl)-3,5-dimethyl-1H-pyrazol-4-yl]-7-fluoroquinoline-2,4-dicarboxamide,
N⁴-[1-(4-cyano-2,6-difluorobenzyl)-3,5-dimethyl-1H-pyrazol-4-yl]-5-fluoroquinoline-2,4-dicarboxamide,
6-bromo-N⁴-[1-(4-cyano-2,6-difluorobenzyl)-3,5-dimethyl-1H-pyrazol-4-yl]quinoline-2,4-dicarboxamide,
N⁴-[1-(4-cyano-2,6-difluorobenzyl)-5-methyl-3-(trifluoromethyl)-1H-pyrazol-4-yl]-7-fluoroquinoline-2,4-dicarboxamide,
N⁴-[1-(4-cyano-2,6-difluorobenzyl)-5-methyl-3-(trifluoromethyl)-1H-pyrazol-4-yl]quinoline-2,4-dicarboxamide,
N⁴-[1-(4-cyano-2,6-difluorobenzyl)-5-methyl-3-(trifluoromethyl)-1H-pyrazol-4-yl]-5-fluoroquinoline-2,4-dicarboxamide,
6-chloro-N⁴-[1-(4-cyano-2, 6-difluorobenzyl)-5-methyl-3-(trifluoromethyl)-1H-pyrazol-4-yl]-7-fluoroquinoline-2,4-dicarboxamide,
6-bromo-N⁴-[1-(4-cyano-2,6-difluorobenzyl)-5-methyl-3-(trifluoromethyl)-1H-pyrazol-4-yl]quinoline-2,4-dicarboxamide,
7-fluoro-N⁴-{5-methyl-1-[4-(1H-tetrazol-5-yl)benzyl]-3-(trifluoromethyl)-1H-pyrazol-4-yl}quinoline-2,4-dicarboxamide,
7-fluoro-N⁴-{1-[4-(methoxymethyl) benzyl]-5-methyl-3-(trifluoromethyl)-1H-pyrazol-4-yl}quinoline-2,4-dicarboxamide,
5-fluoro-N⁴-{1-[4-(methoxymethyl) benzyl]-5-methyl-3-(trifluoromethyl)-1H-pyrazol-4-yl}quinoline-2,4-dicarboxamide,
N⁴-{1-[4-(cyanomethyl)benzyl]-5-methyl-3-(trifluoromethyl)-1H-pyrazol-4-yl}-7-fluoroquinoline-2,4-dicarboxamide,
6-bromo-N-(3,5-dimethyl-1-{[5-(methylcarbamoyl)-1,2,4-oxadiazol-3-yl]methyl}-1H-pyrazol-4-yl)-2-(trifluoromethyl)quinoline-4-carboxamide,
tert-butyl 4-[(4-{[(2-carbamoylquinolin-4-yl)carbonyl]amino}-3,5-dimethyl-1H-pyrazol-1-yl)methyl]piperidine-1-carboxylate,
N⁴-[3,5-dimethyl-1-(piperidin-4-ylmethyl)-1H-pyrazol-4-yl]quinoline-2,4-dicarboxamide,
N⁴-(1-{[1-(ethylsulfonyl)piperidin-4-yl]methyl}-3,5-dimethyl-1H-pyrazol-4-yl)quinoline-2,4-dicarboxamide,
6-chloro-7-fluoro-N⁴-{5-methyl-1-[(5-methyl-1,2-oxazol-3-yl)methyl]-3-(trifluoromethyl)-1H-pyrazol-4-yl}quinoline-2,4-dicarboxamide,
6-chloro-N⁴-{1-[(5-ethyl-1,2,4-oxadiazol-3-yl)methyl]-5-methyl-3-(trifluoromethyl)-1H-pyrazol-4-yl}-7-fluoroquinoline-2,4-dicarboxamide,
6-chloro-N⁴-{1-[(3-ethyl-1,2-oxazol-5-yl)methyl]-5-methyl-3-(trifluoromethyl)-1H-pyrazol-4-yl}-7-fluoroquinoline-2,4-dicarboxamide,
6-chloro-N⁴-{3,5-dimethyl-1-[(3-methyl-1,2-oxazol-5-yl)methyl]-1H-pyrazol-4-yl}-7-fluoroquinoline-2,4-dicarboxamide,
6-chloro-N⁴-{3,5-dimethyl-1-[(5-methyl-1,2-oxazol-3-yl)methyl]-1H-pyrazol-4-yl}-7-fluoroquinoline-2,4-dicarboxamide,
6-chloro-N⁴-[1-{[1-(ethylsulfonyl)piperidin-4-yl]methyl}-5-methyl-3-(trifluoromethyl)-1H-pyrazol-4-yl]-7-fluoroquinoline-2,4-dicarboxamide,
6-chloro-N⁴-{1-[(3-ethyl-1,2-oxazol-5-yl)methyl]-3,5-dimethyl-1H-pyrazol-4-yl}-7-fluoroquinoline-2,4-dicarboxamide,
6-chloro-N⁴-(1-{[1-(ethylsulfonyl)piperidin-4-yl]methyl}-3,5-dimethyl-1H-pyrazol-4-yl)-7-fluoroquinoline-2,4-dicarboxamide,
6-chloro-N⁴-(3,5-dimethyl-1-{[3-(propan-2-yl)-1,2-oxazol-5-yl]methyl}-1H-pyrazol-4-yl)-7-fluoroquinoline-2,4-dicarboxamide,
6-chloro-N⁴-{1-[(5-cyclopropyl-1,2-oxazol-3-yl)methyl]-3,5-dimethyl-1H-pyrazol-4-yl}-7-fluoroquinoline-2,4-dicarboxamide,
N⁴-{1-[(5-cyclopropyl-1,2-oxazol-3-yl)methyl]-3,5-dimethyl-1H-pyrazol-4-yl}-7-fluoroquinoline-2,4-dicarboxamide,
N⁴-{1-[(5-cyclopropyl-1,2-oxazol-3-yl)methyl]-3,5-dimethyl-1H-pyrazol-4-yl}quinoline-2,4-dicarboxamide,
6-chloro-7-fluoro-N⁴-{5-methyl-1-[(3-methyl-1,2-oxazol-5-yl)methyl]-3-(trifluoromethyl)-1H-pyrazol-4-yl}quinoline-2,4-dicarboxamide,
6-chloro-N⁴-{1-[(5-cyano-2-thienyl)methyl]-5-methyl-3-(trifluoromethyl)-1H-pyrazol-4-yl}-7-fluoroquinoline-2,4-dicarboxamide,
N⁴-{1-[(5-cyano-2-thienyl)methyl]-5-methyl-3-(trifluoromethyl)-1H-pyrazol-4-yl}-7-fluoroquinoline-2,4-dicarboxamide,
N⁴-{1-[(5-cyanopyrimidin-2-yl)methyl]-5-methyl-3-(trifluoromethyl)-1H-pyrazol-4-yl}-7-fluoroquinoline-2,4-dicarboxamide,
N⁴-{1-[(5-cyanopyrimidin-2-yl)methyl]-5-methyl-3-(trifluoromethyl)-1H-pyrazol-4-yl}-5-fluoroquinoline-2,4-dicarboxamide,
N⁴-[1-(4-cyanobenzyl)-5-methyl-3-(trifluoromethyl)-1H-pyrazol-4-yl]-6-hydroxyquinoline-2,4-dicarboxamide,
N⁴-[1-(4-cyanobenzyl)-5-methyl-3-(trifluoromethyl)-1H-pyrazol-4-yl]-N²-(methylsulfonyl)quinoline-2,4-dicarboxamide,
N⁴-{1-[(6-cyanopyridazin-3-yl)methyl]-5-methyl-3-(trifluoromethyl)-1H-pyrazol-4-yl}-7-fluoroquinoline-2,4-dicarboxamide,
N-{1-[4-(allylcarbamoyl)benzyl]-3,5-dimethyl-1H-pyrazol-4-yl}-6-bromo-2-(trifluoromethyl)quinoline-4-carboxamide,
6-bromo-N-[1-(4-{[(2R)-2-hydroxypropyl]carbamoyl}benzyl)-3,5-dimethyl-1H-pyrazol-4-yl]-2-(trifluoromethyl)quinoline-4-carboxamide,
6-bromo-N-(3,5-dimethyl-1-{4-[(pyridin-4-ylmethyl)carbamoyl]benzyl}-1H-pyrazol-4-yl)-2-(trifluoromethyl)quinoline-4-carboxamide,
6-bromo-N-(1-{4-[(cyanomethyl)carbamoyl]benzyl}-3,5-dimethyl-1H-pyrazol-4-yl)-2-(trifluoromethyl)quinoline-4-carboxamide,
6-bromo-N-{1-[4-(cyclopropylcarbamoyl)benzyl]-3,5-dimethyl-1H-pyrazol-4-yl}-2-(trifluoromethyl)quinoline-4-carboxamide,
6-bromo-N-{1-[(6-methoxypyridin-3-yl)methyl]-3,5-dimethyl-1H-pyrazol-4-yl}-2-(trifluoromethyl)quinoline-4-carboxamide,
6-bromo-N-{1-[(6-methoxypyridin-3-yl)methyl]-5-methyl-3-(trifluoromethyl)-1H-pyrazol-4-yl}-2-(trifluoromethyl)quinoline-4-carboxamide,
6-bromo-N-{1-[4-(ethylcarbamoyl)benzyl]-3,5-dimethyl-1H-pyrazol-4-yl}-2-(trifluoromethyl)quinoline-4-carboxamide,
6-bromo-N-[3,5-dimethyl-1-(4-{[(1-methyl-1H-pyrazol-3-yl)methyl]carbamoyl}benzyl)-1H-pyrazol-4-yl]-2-(trifluoromethyl)quinoline-4-carboxamide,
6-bromo-N-(1-{4-[(2-fluoroethyl)carbamoyl]benzyl}-3,5-dimethyl-1H-pyrazol-4-yl)-2-(trifluoromethyl)quinoline-4-carboxamide,
6-bromo-N-{3,5-dimethyl-1-[4-(pyrrolidin-1-ylcarbonyl)benzyl]-1H-pyrazol-4-yl}-2-(trifluoromethyl)quinoline-4-carboxamide,
6-bromo-N-[1-(4-methoxybenzyl)-5-methyl-3-(trifluoromethyl)-1H-pyrazol-4-yl]-2-(trifluoromethyl)quinoline-4-carboxamide,
6-bromo-N-[1-(4-fluorobenzyl)-3-methyl-5-(methylcarbamoyl)-1H-pyrazol-4-yl]-2-(trifluoromethyl)quinoline-4-carboxamide,
N-{1-[4-(azetidin-1-ylcarbonyl)benzyl]-3,5-dimethyl-1H-pyrazol-4-yl}-6-bromo-2-(trifluoromethyl)quinoline-4-carboxamide,
6-bromo-N-(1-{4-[(2-carbamoylphenyl)carbamoyl]benzyl}-3,5-dimethyl-1H-pyrazol-4-yl)-2-(trifluoromethyl)quinoline-4-carboxamide,
6-bromo-N-[1-(3-fluoro-4-methoxybenzyl)-3,5-dimethyl-1H-pyrazol-4-yl]-2-(trifluoromethyl)quinoline-4-carboxamide,
6-bromo-N-(1-{4-[(2-chlorophenyl)carbamoyl]benzyl}-3,5-dimethyl-1H-pyrazol-4-yl)-2-(trifluoromethyl)quinoline-4-carboxamide,
6-bromo-N-(3,5-dimethyl-1-{[5-(phenylcarbamoyl)pyridin-2-yl]methyl}-1H-pyrazol-4-yl)-2-(trifluoromethyl)quinoline-4-carboxamide,
6-bromo-N-(3,5-dimethyl-1-{[5-(phenylcarbamoyl)pyridin-2-yl]methyl}-1H-pyrazol-4-yl)-2-(trifluoromethyl)quinoline-4-carboxamide,
6-bromo-N-{3,5-dimethyl-1-[4-({2-[methyl(methylcarbamoyl)amino]ethyl}carbamoyl)benzyl]-1H-pyrazol-4-yl}-2-(trifluoromethyl)quinoline-4-carboxamide,
6-bromo-N-[1-(4-{[4-(dimethylamino)-4-oxobutyl]carbamoyl}benzyl)-3,5-dimethyl-1H-pyrazol-4-yl]-2-(trifluoromethyl)quinoline-4-carboxamide,
6-bromo-N-{3,5-dimethyl-1-[4-(pyridin-3-ylcarbamoyl)benzyl]-1H-pyrazol-4-yl}-2-(trifluoromethyl)quinoline-4-carboxamide,
6-bromo-N-[1-(4-{[(2S)-2-hydroxypropyl]carbamoyl}benzyl)-3,5-dimethyl-1H-pyrazol-4-yl]-2-(trifluoromethyl)quinoline-4-carboxamide,
6-bromo-N-{3,5-dimethyl-1-[4-(1,3-thiazol-2-ylcarbamoyl)benzyl]-1H-pyrazol-4-yl}-2-(trifluoromethyl)quinoline-4-carboxamide,
6,8-dichloro-N-[1-(4-hydroxybenzyl)-3,5-dimethyl-1H-pyrazol-4-yl]-2-(trifluoromethyl)quinoline-4-carboxamide,
6-bromo-N-[1-(4-{[2-(1,1-dioxido-1-thia-6-azaspiro[3.3]hept-6-yl)ethyl]carbamoyl}benzyl)-3,5-dimethyl-1H-pyrazol-4-yl]-2-(trifluoromethyl)quinoline-4-carboxamide,
6-bromo-N-(1-{4-[(3-hydroxypropyl)carbamoyl]benzyl}-3,5-dimethyl-1H-pyrazol-4-yl)-2-(trifluoromethyl)quinoline-4-carboxamide,
N-{3,5-dimethyl-1-[4-(methylsulfonyl)benzyl]-1H-pyrazol-4-yl}-2-methoxyquinoline-4-carboxamide,
6-bromo-N-(1-{4-[(trans-4-hydroxycyclohexyl)carbamoyl]benzyl}-3,5-dimethyl-1H-pyrazol-4-yl)-2-(trifluoromethyl)quinoline-4-carboxamide,
6-bromo-N-(1-{4-[(2-cyanoethyl)(2-methoxyethyl)carbamoyl]benzyl}-3,5-dimethyl-1H-pyrazol-4-yl)-2-(trifluoromethyl)quinoline-4-carboxamide,
6-bromo-N-{3,5-dimethyl-1-[4-(1H-pyrazol-3-ylcarbamoyl)benzyl]-1H-pyrazol-4-yl}-2-(trifluoromethyl)quinoline-4-carboxamide,
6-bromo-N-{1-[(6-cyanopyridin-3-yl)methyl]-5-methyl-3-(trifluoromethyl)-1H-pyrazol-4-yl}-2-(trifluoromethyl)quinoline-4-carboxamide,
rel-6-bromo-N-[1-(4-{[(2R,3R)-3-hydroxybutan-2-yl]carbamoyl}benzyl)-3,5-dimethyl-1H-pyrazol-4-yl]-2-(trifluoromethyl)quinoline-4-carboxamide,
6-bromo-N-(3,5-dimethyl-1-{4-[(tetrahydrofuran-2-ylmethyl)carbamoyl]benzyl}-1H-pyrazol-4-yl)-2-(trifluoromethyl)quinoline-4-carboxamide,
6-bromo-N-(3,5-dimethyl-1-{4-[(2,2,2-trifluoroethyl)carbamoyl]benzyl}-1H-pyrazol-4-yl)-2-(trifluoromethyl)quinoline-4-carboxamide,
6-bromo-N-[3,5-dimethyl-1-(4-{[4-(1H-tetrazol-5-yl)benzyl]carbamoyl}benzyl)-1H-pyrazol-4-yl]-2-(trifluoromethyl)quinoline-4-carboxamide,
6-bromo-N-(3,5-dimethyl-1-{4-[(tetrahydro-2H-pyran-2-ylmethyl)carbamoyl]benzyl}-1H-pyrazol-4-yl)-2-(trifluoromethyl)quinoline-4-carboxamide,
6-bromo-N-(1-{4-[(cis-4-hydroxycyclohexyl)carbamoyl]benzyl}-3,5-dimethyl-1H-pyrazol-4-yl)-2-(trifluoromethyl)quinoline-4-carboxamide,
6-bromo-N-[1-(4-{[2-(4-methoxyphenyl)ethyl]carbamoyl}benzyl)-3,5-dimethyl-1H-pyrazol-4-yl]-2-(trifluoromethyl)quinoline-4-carboxamide,
6-bromo-N-{3,5-dimethyl-1-[4-(pyridin-2-ylcarbamoyl)benzyl]-1H-pyrazol-4-yl}-2-(trifluoromethyl)quinoline-4-carboxamide,
6-bromo-N-[1-({5-[(2-hydroxyethyl)carbamoyl]pyridin-2-yl}methyl)-5-methyl-3-(trifluoromethyl)-1H-pyrazol-4-yl]-2-(trifluoromethyl)quinoline-4-carboxamide,
6-bromo-N-(1-{4-[(3-chlorophenyl)carbamoyl]benzyl}-3,5-dimethyl-1H-pyrazol-4-yl)-2-(trifluoromethyl)quinoline-4-carboxamide,
N-(1-{4-[(3-amino-3-oxopropyl)carbamoyl]benzyl}-3,5-dimethyl-1H-pyrazol-4-yl)-6-bromo-2-(trifluoromethyl)quinoline-4-carboxamide,
6-bromo-N-(3,5-dimethyl-1-{4-[(pyridin-2-ylmethyl)carbamoyl]benzyl}-1H-pyrazol-4-yl)-2-(trifluoromethyl)quinoline-4-carboxamide,
6-bromo-N-[1-(4-{[4-(dimethylamino)phenyl]carbamoyl}benzyl)-3,5-dimethyl-1H-pyrazol-4-yl]-2-(trifluoromethyl)quinoline-4-carboxamide,
6-bromo-N-(3,5-dimethyl-1-{[5-(methylcarbamoyl)pyridin-2-yl]methyl}-1H-pyrazol-4-yl)-2-(trifluoromethyl)quinoline-4-carboxamide,
6,8-dichloro-N-[1-(2-hydroxybenzyl)-3,5-dimethyl-1H-pyrazol-4-yl]-2-(trifluoromethyl)quinoline-4-carboxamide,
6-bromo-N-(3,5-dimethyl-1-{[1-(methylsulfonyl)piperidin-4-yl]methyl}-1H-pyrazol-4-yl)-2-(trifluoromethyl)quinoline-4-carboxamide,
6-bromo-N-[1-({5-[(cyclopropylmethyl)carbamoyl]pyridin-2-yl}methyl)-3,5-dimethyl-1H-pyrazol-4-yl]-2-(trifluoromethyl)quinoline-4-carboxamide,
6-bromo-N-{1-[(3-cyanopyridin-4-yl)methyl]-3,5-dimethyl-1H-pyrazol-4-yl}-2-(trifluoromethyl)quinoline-4-carboxamide,
N-[1-(4-cyanobenzyl)-5-methyl-3-(trifluoromethyl)-1H-pyrazol-4-yl]-2-methoxyquinoline-4-carboxamide,
6-bromo-N-{1-[(5-carbamoylpyridin-2-yl)methyl]-5-methyl-3-(trifluoromethyl)-1H-pyrazol-4-yl}-2-(trifluoromethyl)quinoline-4-carboxamide,
6-bromo-N-(1-{4-[(5-hydroxypentyl)carbamoyl]benzyl}-3,5-dimethyl-1H-pyrazol-4-yl)-2-(trifluoromethyl)quinoline-4-carboxamide,
6-bromo-N-[5-methyl-1-{[5-(methylcarbamoyl)pyridin-2-yl]methyl}-3-(trifluoromethyl)-1H-pyrazol-4-yl]-2-(trifluoromethyl)quinoline-4-carboxamide,
6-bromo-N-(1-{4-[(cyclopropylmethyl)carbamoyl]benzyl}-3,5-dimethyl-1H-pyrazol-4-yl)-2-(trifluoromethyl)quinoline-4-carboxamide,
6-bromo-N-(1-{4-[(2, 2-difluoroethyl)carbamoyl]benzyl}-3,5-dimethyl-1H-pyrazol-4-yl)-2-(trifluoromethyl)quinoline-4-carboxamide,
6-bromo-N-[1-(4-{[(2R)-2-(hydroxymethyl)pyrrolidin-1-yl]carbonyl}benzyl)-3,5-dimethyl-1H-pyrazol-4-yl]-2-(trifluoromethyl)quinoline-4-carboxamide,
N⁴-{1-[(3-cyanopyridin-2-yl)methyl]-3,5-dimethyl-1H-pyrazol-4-yl}quinoline-2,4-dicarboxamide,
6-bromo-N-(1-{4-[(4-hydroxybutyl)carbamoyl]benzyl}-3,5-dimethyl-1H-pyrazol-4-yl)-2-(trifluoromethyl)quinoline-4-carboxamide,
6-bromo-N-[1-(4-{[2-(2-fluorophenoxy)ethyl]carbamoyl}benzyl)-3,5-dimethyl-1H-pyrazol-4-yl]-2-(trifluoromethyl)quinoline-4-carboxamide,
6-bromo-N-(1-{4-[(2-hydroxy-2-methylpropyl)carbamoyl]benzyl}-3,5-dimethyl-1H-pyrazol-4-yl)-2-(trifluoromethyl)quinoline-4-carboxamide,
N⁴-{1-[(6-cyanopyridin-3-yl)methyl]-3,5-dimethyl-1H-pyrazol-4-yl}-6-fluoroquinoline-2,4-dicarboxamide,
6-bromo-N-(1-{4-[(1,1-dioxido-1-thia-6-azaspiro[3.3]hept-6-yl)carbonyl]benzyl}-3,5-dimethyl-1H-pyrazol-4-yl)-2-(trifluoromethyl)quinoline-4-carboxamide,
N-[1-(4-fluorobenzyl)-3,5-dimethyl-1H-pyrazol-4-yl]-6-nitro-2-(trifluoromethyl)quinoline-4-carboxamide,
2-methoxy-N-[1-(4-methoxybenzyl)-5-methyl-3-(trifluoromethyl)-1H-pyrazol-4-yl]quinoline-4-carboxamide,
6-bromo-N-[1-(4-{[3-(1H-imidazol-1-yl)propyl]carbamoyl}benzyl)-3,5-dimethyl-1H-pyrazol-4-yl]-2-(trifluoromethyl)quinoline-4-carboxamide,
N-[1-(4-cyanobenzyl)-3,5-dimethyl-1H-pyrazol-4-yl]-2-(dimethylamino)quinoline-4-carboxamide,
6-bromo-N-[1-({5-[(2-hydroxyethyl)carbamoyl]pyridin-2-yl}methyl)-3,5-dimethyl-1H-pyrazol-4-yl]-2-(trifluoromethyl)quinoline-4-carboxamide,
N⁴-(3,5-dimethyl-1-{[1-(methylsulfonyl)piperidin-4-yl]methyl}-1H-pyrazol-4-yl)quinoline-2,4-dicarboxamide,
6-bromo-N-(1-{4-[(1,3-dihydroxy-2-methylpropan-2-yl)carbamoyl]benzyl}-3,5-dimethyl-1H-pyrazol-4-yl)-2-(trifluoromethyl)quinoline-4-carboxamide,
N-[3,5-dimethyl-1-(4-vinylbenzyl)-1H-pyrazol-4-yl]-2,6-dimethylquinoline-4-carboxamide,
6-bromo-N-{1-[4-({2-[(cyclopropylcarbonyl)amino]ethyl}carbamoyl)benzyl]-3,5-dimethyl-1H-pyrazol-4-yl}-2-(trifluoromethyl)quinoline-4-carboxamide,
6-bromo-N-[1-(4-{[4-(hydroxymethyl)piperidin-1-yl]carbonyl}benzyl)-3,5-dimethyl-1H-pyrazol-4-yl]-2-(trifluoromethyl)quinoline-4-carboxamide,
N⁴-{1-[2-(4-fluorophenyl)ethyl]-3,5-dimethyl-1H-pyrazol-4-yl}quinoline-2,4-dicarboxamide,
6-bromo-N-[1-(4-{[(2S)-1-hydroxypropan-2-yl]carbamoyl}benzyl)-3,5-dimethyl-1H-pyrazol-4-yl]-2-(trifluoromethyl)quinoline-4-carboxamide,
N-{1-[(3-cyanopyridin-2-yl)methyl]-3,5-dimethyl-1H-pyrazol-4-yl}-2-methoxyquinoline-4-carboxamide,
N⁴-[1-(2, 3-dihydro-1,4-benzodioxin-2-ylmethyl)-3,5-dimethyl-1H-pyrazol-4-yl]quinoline-2,4-dicarboxamide,
methyl N-(4-{[4-({[6-bromo-2-(trifluoromethyl)quinolin-4-yl]carbonyl}amino)-3,5-dimethyl-1H-pyrazol-1-yl]methyl}benzoyl)-L-threoninate,
6-bromo-N-{1-[(3-cyanopyridin-4-yl)methyl]-5-methyl-3-(trifluoromethyl)-1H-pyrazol-4-yl}-2-(trifluoromethyl)quinoline-4-carboxamide,
6-bromo-N-[3,5-dimethyl-1-(4-{[2-(1H-pyrazol-1-yl)ethyl]carbamoyl}benzyl)-1H-pyrazol-4-yl]-2-(trifluoromethyl)quinoline-4-carboxamide,
6-bromo-N-(1-{4-[(5-hydroxy-4,4-dimethylpentyl)carbamoyl]benzyl}-3,5-dimethyl-1H-pyrazol-4-yl)-2-(trifluoromethyl)quinoline-4-carboxamide,
6-bromo-N-(3,5-dimethyl-1-{4-[(4-methylbenzyl)carbamoyl]benzyl}-1H-pyrazol-4-yl)-2-(trifluoromethyl)quinoline-4-carboxamide,
2-methoxy-N-{1-[3-(4-methoxyphenyl)propyl]-3,5-dimethyl-1H-pyrazol-4-yl}quinoline-4-carboxamide,
6-bromo-N-(3,5-dimethyl-1-{4-[(2-thienylmethyl)carbamoyl]benzyl}-1H-pyrazol-4-yl)-2-(trifluoromethyl)quinoline-4-carboxamide,
6-bromo-N-(3,5-dimethyl-1-{[1-(methylcarbamoyl)piperidin-4-yl]methyl}-1H-pyrazol-4-yl)-2-(trifluoromethyl)quinoline-4-carboxamide,
6-bromo-N-[1-(4-{[(2S)-2, 3-dihydroxypropyl]carbamoyl}benzyl)-3,5-dimethyl-1H-pyrazol-4-yl]-2-(trifluoromethyl)quinoline-4-carboxamide,
N-[1-(4-bromobenzyl)-3,5-dimethyl-1H-pyrazol-4-yl]-2,6-dimethylquinoline-4-carboxamide,
6-bromo-N-[3,5-dimethyl-1-(tetrahydro-2H-pyran-4-ylmethyl)-1H-pyrazol-4-yl]-2-(trifluoromethyl)quinoline-4-carboxamide,
6-bromo-N-[1-({5-[(2-methoxyethyl)carbamoyl]pyridin-2-yl}methyl)-5-methyl-3-(trifluoromethyl)-1H-pyrazol-4-yl]-2-(trifluoromethyl)quinoline-4-carboxamide,
6-bromo-N-[1-(4-{[2-(1H-imidazol-1-yl)ethyl]carbamoyl}benzyl)-3,5-dimethyl-1H-pyrazol-4-yl]-2-(trifluoromethyl)quinoline-4-carboxamide,
6-amino-N-[1-(4-fluorobenzyl)-3,5-dimethyl-1H-pyrazol-4-yl]-2-(trifluoromethyl)quinoline-4-carboxamide,
6-bromo-N-{1-[(2-cyanopyridin-3-yl)methyl]-3,5-dimethyl-1H-pyrazol-4-yl}-2-(trifluoromethyl)quinoline-4-carboxamide,
6-bromo-N-(1-{4-[(4-fluorobenzyl)carbamoyl]benzyl}-3,5-dimethyl-1H-pyrazol-4-yl)-2-(trifluoromethyl)quinoline-4-carboxamide,
6-bromo-N-(1-{4-[(3-methoxypropyl)carbamoyl]benzyl}-3,5-dimethyl-1H-pyrazol-4-yl)-2-(trifluoromethyl)quinoline-4-carboxamide,
N-{1-[4-({2-[bis(2-hydroxyethyl)amino]ethyl}carbamoyl)benzyl]-3,5-dimethyl-1H-pyrazol-4-yl}-6-bromo-2-(trifluoromethyl)quinoline-4-carboxamide,
6-bromo-N-(1-{4-[(1,3-dihydroxypropan-2-yl)carbamoyl]benzyl}-3,5-dimethyl-1H-pyrazol-4-yl)-2-(trifluoromethyl)quinoline-4-carboxamide,
6-bromo-N-[1-(4-{[(2,2-difluorocyclopropyl)methyl]carbamoyl}benzyl)-3,5-dimethyl-1H-pyrazol-4-yl]-2-(trifluoromethyl)quinoline-4-carboxamide,
(±)-6-bromo-N-[3,5-dimethyl-1-({5-[(oxetan-2-ylmethyl)carbamoyl]pyridin-2-yl}methyl)-1H-pyrazol-4-yl]-2-(trifluoromethyl)quinoline-4-carboxamide,
6-bromo-N-[3,5-dimethyl-1-(4-{[(1-methyl-1H-imidazol-4-yl)methyl]carbamoyl}benzyl)-1H-pyrazol-4-yl]-2-(trifluoromethyl)quinoline-4-carboxamide,
6-bromo-N-[1-(4-fluorobenzyl)-5-methyl-3-(methylcarbamoyl)-1H-pyrazol-4-yl]-2-(trifluoromethyl)quinoline-4-carboxamide,
N-[1-(4-cyano-3-fluorobenzyl)-3,5-dimethyl-1H-pyrazol-4-yl]-2,6-dimethylquinoline-4-carboxamide,
N-{1-[(6-cyanopyridin-3-yl)methyl]-5-methyl-3-(trifluoromethyl)-1H-pyrazol-4-yl}-2-methoxyquinoline-4-carboxamide,
6-bromo-N-{1-[2-(4-fluorophenyl)ethyl]-3,5-dimethyl-1H-pyrazol-4-yl}-2-(trifluoromethyl)quinoline-4-carboxamide,
6-bromo-N-(3,5-dimethyl-1-{4-[(1,3-thiazol-2-ylmethyl)carbamoyl]benzyl}-1H-pyrazol-4-yl)-2-(trifluoromethyl)quinoline-4-carboxamide,
6-bromo-N-[1-({5-[(3-hydroxypropyl)carbamoyl]pyridin-2-yl}methyl)-3,5-dimethyl-1H-pyrazol-4-yl]-2-(trifluoromethyl)quinoline-4-carboxamide,
6-bromo-N-(1-{4-[(3-methoxybenzyl)carbamoyl]benzyl}-3,5-dimethyl-1H-pyrazol-4-yl)-2-(trifluoromethyl)quinoline-4-carboxamide,
6-bromo-N-[3,5-dimethyl-1-(4-{[3-(morpholin-4-yl)propyl]carbamoyl}benzyl)-1H-pyrazol-4-yl]-2-(trifluoromethyl)quinoline-4-carboxamide,
6-bromo-N-(1-{4-[(3-hydroxy-2,2-dimethylpropyl)carbamoyl]benzyl}-3,5-dimethyl-1H-pyrazol-4-yl)-2-(trifluoromethyl)quinoline-4-carboxamide,
6-bromo-N-[3-carbamoyl-1-(4-fluorobenzyl)-5-methyl-1H-pyrazol-4-yl]-2-(trifluoromethyl)quinoline-4-carboxamide,
6-bromo-N-[1-(4-{[1,3-dihydroxy-2-(hydroxymethyl)propan-2-yl]carbamoyl}benzyl)-3,5-dimethyl-1H-pyrazol-4-yl]-2-(trifluoromethyl)quinoline-4-carboxamide,
6-bromo-N-(3,5-dimethyl-1-{4-[(2-phenoxyethyl)carbamoyl]benzyl}-1H-pyrazol-4-yl)-2-(trifluoromethyl)quinoline-4-carboxamide,
6-bromo-N-{1-[(5-carbamoylpyridin-2-yl)methyl]-3,5-dimethyl-1H-pyrazol-4-yl}-2-(trifluoromethyl)quinoline-4-carboxamide,
6-bromo-N-[3,5-dimethyl-1-(4-{[2-(pyridin-2-yl)ethyl]carbamoyl}benzyl)-1H-pyrazol-4-yl]-2-(trifluoromethyl)quinoline-4-carboxamide,
6-bromo-N-(3,5-dimethyl-1-{4-[(2-phenylethyl)carbamoyl]benzyl}-1H-pyrazol-4-yl)-2-(trifluoromethyl)quinoline-4-carboxamide,
6-bromo-N-{3,5-dimethyl-1-[4-(prop-2-yn-1-ylcarbamoyl)benzyl]-1H-pyrazol-4-yl}-2-(trifluoromethyl)quinoline-4-carboxamide,
6-bromo-N-[3,5-dimethyl-1-(4-{[(1-methyl-1H-imidazol-5-yl)methyl]carbamoyl}benzyl)-1H-pyrazol-4-yl]-2-(trifluoromethyl)quinoline-4-carboxamide,
6,8-dichloro-N-[3,5-dimethyl-1-(tetrahydro-2H-pyran-4-ylmethyl)-1H-pyrazol-4-yl]-2-(trifluoromethyl)quinoline-4-carboxamide,
6-bromo-N-[1-(4-{[2-(2-hydroxyethoxy)ethyl]carbamoyl}benzyl)-3,5-dimethyl-1H-pyrazol-4-yl]-2-(trifluoromethyl)quinoline-4-carboxamide,
N-(1-{4-[bis(2-hydroxyethyl)carbamoyl]benzyl}-3,5-dimethyl-1H-pyrazol-4-yl)-6-bromo-2-(trifluoromethyl)quinoline-4-carboxamide,
6-bromo-N-[1-(4-{[2-(1-hydroxycyclopentyl)ethyl](methyl)carbamoyl}benzyl)-3,5-dimethyl-1H-pyrazol-4-yl]-2-(trifluoromethyl)quinoline-4-carboxamide,
6-bromo-N-(1-{4-[(3-cyanoazetidin-1-yl)carbonyl]benzyl}-3,5-dimethyl-1H-pyrazol-4-yl)-2-(trifluoromethyl)quinoline-4-carboxamide,
N⁴-{1-[2-(3-fluorophenyl)ethyl]-3,5-dimethyl-1H-pyrazol-4-yl}quinoline-2,4-dicarboxamide,
6-bromo-N-(1-{4-[(2-fluorobenzyl)carbamoyl]benzyl}-3,5-dimethyl-1H-pyrazol-4-yl)-2-(trifluoromethyl)quinoline-4-carboxamide,
6-bromo-N-[3,5-dimethyl-1-(4-{[2-(piperidin-1-yl)ethyl]carbamoyl}benzyl)-1H-pyrazol-4-yl]-2-(trifluoromethyl)quinoline-4-carboxamide,
N-(1-{[5-(benzylcarbamoyl)pyridin-2-yl]methyl}-3,5-dimethyl-1H-pyrazol-4-yl)-6-bromo-2-(trifluoromethyl)quinoline-4-carboxamide,
6-bromo-N-[1-(4-{[(2R)-2, 3-dihydroxypropyl]carbamoyl}benzyl)-3,5-dimethyl-1H-pyrazol-4-yl]-2-(trifluoromethyl)quinoline-4-carboxamide,
N-{1-[(2-cyanopyridin-3-yl)methyl]-3,5-dimethyl-1H-pyrazol-4-yl}-2-methoxyquinoline-4-carboxamide,
6-bromo-N-[3,5-dimethyl-1-(4-{[(1-methyl-1H-pyrazol-4-yl)methyl]carbamoyl}benzyl)-1H-pyrazol-4-yl]-2-(trifluoromethyl)quinoline-4-carboxamide,
methyl 4-[(4-{[(2-carbamoylquinolin-4-yl)carbonyl]amino}-3,5-dimethyl-1H-pyrazol-1-yl)methyl]piperidine-1-carboxylate,
N-{1-[(1-acetylpiperidin-4-yl)methyl]-3,5-dimethyl-1H-pyrazol-4-yl}-6-bromo-2-(trifluoromethyl)quinoline-4-carboxamide,
6-bromo-N-[3,5-dimethyl-1-(4-{[3-(methylamino)-3-oxopropyl]carbamoyl}benzyl)-1H-pyrazol-4-yl]-2-(trifluoromethyl)quinoline-4-carboxamide,
6-bromo-N-{3,5-dimethyl-1-[4-(pyrimidin-4-ylcarbamoyl)benzyl]-1H-pyrazol-4-yl}-2-(trifluoromethyl)quinoline-4-carboxamide,
6-bromo-N-{3,5-dimethyl-1-[(5-{[(3-methyloxetan-3-yl)methyl]carbamoyl}pyridin-2-yl)methyl]-1H-pyrazol-4-yl}-2-(trifluoromethyl)quinoline-4-carboxamide,
6-bromo-N-(1-{4-[(2-cyanoethyl)(methyl)carbamoyl]benzyl}-3,5-dimethyl-1H-pyrazol-4-yl)-2-(trifluoromethyl)quinoline-4-carboxamide,
(±)-6-bromo-N-(1-{4-[(3-hydroxypyrrolidin-1-yl)carbonyl]benzyl}-3,5-dimethyl-1H-pyrazol-4-yl)-2-(trifluoromethyl)quinoline-4-carboxamide,
(±)-6-bromo-N-(1-{4-[(3-hydroxypiperidin-1-yl)carbonyl]benzyl}-3,5-dimethyl-1H-pyrazol-4-yl)-2-(trifluoromethyl)quinoline-4-carboxamide,
6-bromo-N-(1-{4-[(3-fluorobenzyl)carbamoyl]benzyl}-3,5-dimethyl-1H-pyrazol-4-yl)-2-(trifluoromethyl)quinoline-4-carboxamide,
6-bromo-N-[3,5-dimethyl-1-(4-{[(2S)-tetrahydrofuran-2-ylmethyl]carbamoyl}benzyl)-1H-pyrazol-4-yl]-2-(trifluoromethyl)quinoline-4-carboxamide,
6-bromo-N-{1-[3-(4-methoxyphenyl)propyl]-3,5-dimethyl-1H-pyrazol-4-yl}-2-(trifluoromethyl)quinoline-4-carboxamide,
(±)-6-bromo-N-(3,5-dimethyl-1-{4-[(2-oxopyrrolidin-3-yl)carbamoyl]benzyl}-1H-pyrazol-4-yl)-2-(trifluoromethyl)quinoline-4-carboxamide,
6-bromo-N-[1-(4-{[3-(dimethylamino)propyl]carbamoyl}benzyl)-3,5-dimethyl-1H-pyrazol-4-yl]-2-(trifluoromethyl)quinoline-4-carboxamide,
N-(1-{4-[(2-acetamidoethyl)carbamoyl]benzyl}-3,5-dimethyl-1H-pyrazol-4-yl)-6-bromo-2-(trifluoromethyl)quinoline-4-carboxamide,
6-acetamido-N-[1-(4-fluorobenzyl)-3,5-dimethyl-1H-pyrazol-4-yl]-2-(trifluoromethyl)quinoline-4-carboxamide,
6-bromo-N-[3,5-dimethyl-1-({5-[(pyridin-3-ylmethyl)carbamoyl]pyridin-2-yl}methyl)-1H-pyrazol-4-yl]-2-(trifluoromethyl)quinoline-4-carboxamide,
methyl 4-{[4-({[6-bromo-2-(trifluoromethyl)quinolin-4-yl]carbonyl}amino)-3,5-dimethyl-1H-pyrazol-1-yl]methyl}piperidine-1-carboxylate,
N-[3,5-dimethyl-1-(4-vinylbenzyl)-1H-pyrazol-4-yl]-2-methoxyquinoline-4-carboxamide,
6-bromo-N-{3,5-dimethyl-1-[4-(pyridin-4-ylcarbamoyl)benzyl]-1H-pyrazol-4-yl}-2-(trifluoromethyl)quinoline-4-carboxamide,
6-bromo-N-{1-[4-(tert-butylcarbamoyl)benzyl]-3,5-dimethyl-1H-pyrazol-4-yl}-2-(trifluoromethyl)quinoline-4-carboxamide,
6-bromo-N-[3,5-dimethyl-1-(tetrahydrofuran-2-ylmethyl)-1H-pyrazol-4-yl]-2-(trifluoromethyl)quinoline-4-carboxamide,
N-[3,5-dimethyl-1-(tetrahydro-2H-pyran-4-ylmethyl)-1H-pyrazol-4-yl]-2,6-dimethylquinoline-4-carboxamide,
6-bromo-N-{1-[(5-{[2-(dimethylamino)ethyl]carbamoyl}pyridin-2-yl)methyl]-5-methyl-3-(trifluoromethyl)-1H-pyrazol-4-yl}-2-(trifluoromethyl)quinoline-4-carboxamide,
6-bromo-N-(3,5-dimethyl-1-{4-[(3-phenylpropyl)carbamoyl]benzyl}-1H-pyrazol-4-yl)-2-(trifluoromethyl)quinoline-4-carboxamide,
6-bromo-N-[1-(4-{[(2R)-1-hydroxypropan-2-yl]carbamoyl}benzyl)-3,5-dimethyl-1H-pyrazol-4-yl]-2-(trifluoromethyl)quinoline-4-carboxamide,
6-bromo-N-[5-(dimethylcarbamoyl)-1-(4-fluorobenzyl)-3-methyl-1H-pyrazol-4-yl]-2-(trifluoromethyl)quinoline-4-carboxamide,
N-{1-[2-(4-fluorophenyl)ethyl]-3,5-dimethyl-1H-pyrazol-4-yl}-2-methoxyquinoline-4-carboxamide,
6-bromo-N-{1-[4-(cyclopentylcarbamoyl)benzyl]-3,5-dimethyl-1H-pyrazol-4-yl}-2-(trifluoromethyl)quinoline-4-carboxamide,
6-bromo-N-(1-{4-[(2-methoxybenzyl)carbamoyl]benzyl}-3,5-dimethyl-1H-pyrazol-4-yl)-2-(trifluoromethyl)quinoline-4-carboxamide,
tert-butyl 4-[(4-{[(2-carbamoylquinolin-4-yl)carbonyl]amino}-3,5-dimethyl-1H-pyrazol-1-yl)methyl]piperidine-1-carboxylate,
6-bromo-N-(1-{4-[(3-carbamoylphenyl)carbamoyl]benzyl}-3,5-dimethyl-1H-pyrazol-4-yl)-2-(trifluoromethyl)quinoline-4-carboxamide,
6-bromo-N-(1-{4-[(2-hydroxyethyl)(methyl)carbamoyl]benzyl}-3,5-dimethyl-1H-pyrazol-4-yl)-2-(trifluoromethyl)quinoline-4-carboxamide,
6-bromo-N-[3,5-dimethyl-1-(4-{methyl[2-(methylamino)-2-oxoethyl]carbamoyl}benzyl)-1H-pyrazol-4-yl]-2-(trifluoromethyl)quinoline-4-carboxamide,
N-{1-[(3-cyanopyridin-4-yl)methyl]-5-methyl-3-(trifluoromethyl)-1H-pyrazol-4-yl}-2-methoxyquinoline-4-carboxamide,
N-{1-[(3-cyanopyridin-4-yl)methyl]-3,5-dimethyl-1H-pyrazol-4-yl}-2-methoxyquinoline-4-carboxamide,
6-bromo-N-(3,5-dimethyl-1-{4-[(3-methylbenzyl)carbamoyl]benzyl}-1H-pyrazol-4-yl)-2-(trifluoromethyl)quinoline-4-carboxamide,
6-bromo-N-{3,5-dimethyl-1-[(5-{[2-(morpholin-4-yl)ethyl]carbamoyl}pyridin-2-yl)methyl]-1H-pyrazol-4-yl}-2-(trifluoromethyl)quinoline-4-carboxamide,
6-bromo-N-[1-(4-{[4-(2-hydroxyethyl)piperazin-1-yl]carbonyl}benzyl)-3,5-dimethyl-1H-pyrazol-4-yl]-2-(trifluoromethyl)quinoline-4-carboxamide,
6-bromo-N-(3,5-dimethyl-1-{4-[(tetrahydro-2H-pyran-4-ylmethyl)carbamoyl]benzyl}-1H-pyrazol-4-yl)-2-(trifluoromethyl)quinoline-4-carboxamide,
6-bromo-N-[1-{[5-(dimethylcarbamoyl)pyridin-2-yl]methyl}-5-methyl-3-(trifluoromethyl)-1H-pyrazol-4-yl]-2-(trifluoromethyl)quinoline-4-carboxamide,
N-[1-(2-chloro-4-fluorobenzyl)-3,5-dimethyl-1H-pyrazol-4-yl]-2,6-dimethylquinoline-4-carboxamide,
N-{3,5-dimethyl-1-[(5-methyl-1,2-oxazol-3-yl)methyl]-1H-pyrazol-4-yl}-2-methoxyquinoline-4-carboxamide,
6-bromo-N-[5-carbamoyl-1-(4-fluorobenzyl)-3-methyl-1H-pyrazol-4-yl]-2-(trifluoromethyl)quinoline-4-carboxamide,
N-[1-(4-cyanobenzyl)-3,5-dimethyl-1H-pyrazol-4-yl]-6-(3,5-dimethyl-1,2-oxazol-4-yl)-2-(trifluoromethyl)quinoline-4-carboxamide,
N-[1-(2,4-dichlorobenzyl)-3,5-dimethyl-1H-pyrazol-4-yl]-2,6-dimethylquinoline-4-carboxamide,
6-bromo-N-[5-methyl-1-{[5-(morpholin-4-ylcarbonyl)pyridin-2-yl]methyl}-3-(trifluoromethyl)-1H-pyrazol-4-yl]-2-(trifluoromethyl)quinoline-4-carboxamide,
tert-butyl 4-{[4-({[6-bromo-2-(trifluoromethyl)quinolin-4-yl]carbonyl}amino)-3,5-dimethyl-1H-pyrazol-1-yl]methyl}piperidine-1-carboxylate,
N-{1-[2-(3-fluorophenyl)ethyl]-3,5-dimethyl-1H-pyrazol-4-yl}-2-methoxyquinoline-4-carboxamide,
6-bromo-N-{1-[4-(diethylcarbamoyl)benzyl]-3,5-dimethyl-1H-pyrazol-4-yl}-2-(trifluoromethyl)quinoline-4-carboxamide,
6-bromo-N-(1-{4-[(3-hydroxy-3-methylbutyl)carbamoyl]benzyl}-3,5-dimethyl-1H-pyrazol-4-yl)-2-(trifluoromethyl)quinoline-4-carboxamide,
N-{1-[(7-fluoro-2,1,3-benzothiadiazol-4-yl)methyl]-3,5-dimethyl-1H-pyrazol-4-yl}-2,6-dimethylquinoline-4-carboxamide,
6-bromo-N-{3,5-dimethyl-1-[(2-oxo-1,3-oxazolidin-5-yl)methyl]-1H-pyrazol-4-yl}-2-(trifluoromethyl)quinoline-4-carboxamide,
N⁴-{1-[(5-carbamoylpyridin-2-yl)methyl]-3,5-dimethyl-1H-pyrazol-4-yl}-6-fluoroquinoline-2,4-dicarboxamide,
6-bromo-N-[1-(2,3-dihydro-1,4-benzodioxin-2-ylmethyl)-3,5-dimethyl-1H-pyrazol-4-yl]-2-(trifluoromethyl)quinoline-4-carboxamide,
6-bromo-N-(1-{4-[(3-hydroxybenzyl)carbamoyl]benzyl}-3,5-dimethyl-1H-pyrazol-4-yl)-2-(trifluoromethyl)quinoline-4-carboxamide,
6-bromo-N-[3,5-dimethyl-1-(4-{[2-(4-methylphenyl)ethyl]carbamoyl}benzyl)-1H-pyrazol-4-yl]-2-(trifluoromethyl)quinoline-4-carboxamide,
6-bromo-N-(1-{4-[(3-hydroxybutyl)carbamoyl]benzyl}-3,5-dimethyl-1H-pyrazol-4-yl)-2-(trifluoromethyl)quinoline-4-carboxamide,
6-bromo-N-(3,5-dimethyl-1-{4-[(4-methylpiperazin-1-yl)carbonyl]benzyl}-1H-pyrazol-4-yl)-2-(trifluoromethyl)quinoline-4-carboxamide,
N⁴-{1-[(1-acetylpiperidin-4-yl)methyl]-3,5-dimethyl-1H-pyrazol-4-yl}quinoline-2,4-dicarboxamide,
N⁴-[3-(dimethylcarbamoyl)-1-(4-fluorobenzyl)-5-methyl-1H-pyrazol-4-yl]quinoline-2,4-dicarboxamide,
6-bromo-N-[1-(4-{[(1S,2S)-2-hydroxycyclohexyl]carbamoyl}benzyl)-3,5-dimethyl-1H-pyrazol-4-yl]-2-(trifluoromethyl)quinoline-4-carboxamide,
6-bromo-N-[3,5-dimethyl-1-(4-{[2-(1-methylpyrrolidin-2-yl)ethyl]carbamoyl}benzyl)-1H-pyrazol-4-yl]-2-(trifluoromethyl)quinoline-4-carboxamide,
6-bromo-N-(1-{4-[(4-hydroxypiperidin-1-yl)carbonyl]benzyl}-3,5-dimethyl-1H-pyrazol-4-yl)-2-(trifluoromethyl)quinoline-4-carboxamide,
6-bromo-N-{3,5-dimethyl-1-[2-(morpholin-4-yl)ethyl]-1H-pyrazol-4-yl}-2-(trifluoromethyl)quinoline-4-carboxamide,
6-bromo-N-{1-(4-fluorobenzyl)-5-[(2-hydroxyethyl)carbamoyl]-3-methyl-1H-pyrazol-4-yl}-2-(trifluoromethyl)quinoline-4-carboxamide,
6-bromo-N-{3,5-dimethyl-1-[2-(piperidin-1-yl)ethyl]-1H-pyrazol-4-yl}-2-(trifluoromethyl)quinoline-4-carboxamide,
6-bromo-N-(1-{[5-(dimethylcarbamoyl)pyridin-2-yl]methyl}-3,5-dimethyl-1H-pyrazol-4-yl)-2-(trifluoromethyl)quinoline-4-carboxamide,
6-bromo-N-[3,5-dimethyl-1-(piperidin-4-ylmethyl)-1H-pyrazol-4-yl]-2-(trifluoromethyl)quinoline-4-carboxamide,
N-(1-{4-[(2-amino-2-oxoethyl)carbamoyl]benzyl}-3,5-dimethyl-1H-pyrazol-4-yl)-6-bromo-2-(trifluoromethyl)quinoline-4-carboxamide,
6-bromo-N-[1-(4-{[2-(dimethylamino)ethyl](methyl)carbamoyl}benzyl)-3,5-dimethyl-1H-pyrazol-4-yl]-2-(trifluoromethyl)quinoline-4-carboxamide,
ethyl 4-({[6-bromo-2-(trifluoromethyl)quinolin-4-yl]carbonyl}amino)-1-(4-fluorobenzyl)-5-methyl-1H-pyrazole-3-carboxylate,
N-{3,5-dimethyl-1-[(2E)-3-phenylprop-2-en-1-yl]-1H-pyrazol-4-yl}-2,6-dimethylquinoline-4-carboxamide,
6-bromo-N-(1-{4-[(2,2-dimethylpropyl)carbamoyl]benzyl}-3,5-dimethyl-1H-pyrazol-4-yl)-2-(trifluoromethyl)quinoline-4-carboxamide,
6-bromo-N-(1-{4-[(2-hydroxyethyl)(pentyl)carbamoyl]benzyl}-3,5-dimethyl-1H-pyrazol-4-yl)-2-(trifluoromethyl)quinoline-4-carboxamide,
6-bromo-N-(1-{4-[(4-methoxybenzyl)carbamoyl]benzyl}-3,5-dimethyl-1H-pyrazol-4-yl)-2-(trifluoromethyl)quinoline-4-carboxamide,
6-bromo-N-[1-({5-[(1,3-dihydroxypropan-2-yl)carbamoyl]pyridin-2-yl}methyl)-3,5-dimethyl-1H-pyrazol-4-yl]-2-(trifluoromethyl)quinoline-4-carboxamide,
N⁴-[1-(4-fluorobenzyl)-5-methyl-3-(methylcarbamoyl)-1H-pyrazol-4-yl]quinoline-2,4-dicarboxamide,
N-[1-(2-methoxybenzyl)-3,5-dimethyl-1H-pyrazol-4-yl]-2,6-dimethylquinoline-4-carboxamide,
6-bromo-N-{1-[2-(3,5-dimethyl-1H-pyrazol-1-yl)ethyl]-3,5-dimethyl-1H-pyrazol-4-yl}-2-(trifluoromethyl)quinoline-4-carboxamide,
6-bromo-N-(3,5-dimethyl-1-{[5-(morpholin-4-ylcarbonyl)pyridin-2-yl]methyl}-1H-pyrazol-4-yl)-2-(trifluoromethyl)quinoline-4-carboxamide,
6-bromo-N-{1-(4-fluorobenzyl)-3-[(2-hydroxyethyl)carbamoyl]-5-methyl-1H-pyrazol-4-yl}-2-(trifluoromethyl)quinoline-4-carboxamide,
N-[1-(3-fluorobenzyl)-3,5-dimethyl-1H-pyrazol-4-yl]-2,6-dimethylquinoline-4-carboxamide,
6-cyano-N-[1-(4-fluorobenzyl)-3,5-dimethyl-1H-pyrazol-4-yl]-2-methylquinoline-4-carboxamide,
6-bromo-N-[1-(2,5-difluorobenzyl)-3,5-dimethyl-1H-pyrazol-4-yl]-2-(trifluoromethyl)quinoline-4-carboxamide,
6-bromo-N-(1-{[5-({[3-(hydroxymethyl)oxetan-3-yl]methyl}carbamoyl)pyridin-2-yl]methyl}-3,5-dimethyl-1H-pyrazol-4-yl)-2-(trifluoromethyl)quinoline-4-carboxamide,
6-bromo-N-{3,5-dimethyl-1-[4-(2-{[2-(morpholin-4-yl)ethyl]amino}-2-oxoethyl)benzyl]-1H-pyrazol-4-yl}-2-(trifluoromethyl)quinoline-4-carboxamide,
N⁴-[3,5-dimethyl-1-(piperidin-4-ylmethyl)-1H-pyrazol-4-yl]quinoline-2,4-dicarboxamide,
N-[4-{[1-(4-fluorobenzyl)-3,5-dimethyl-1H-pyrazol-4-yl]carbamoyl}-2-(trifluoromethyl)quinolin-6-yl]ethanediamide,
6,8-dichloro-N-{3,5-dimethyl-1-[4-(trifluoromethyl)benzyl]-1H-pyrazol-4-yl}-2-(trifluoromethyl)quinoline-4-carboxamide,
6,8-dichloro-N-[1-(3,5-difluorobenzyl)-3,5-dimethyl-1H-pyrazol-4-yl]-2-(trifluoromethyl)quinoline-4-carboxamide,
N-[5-(dimethylcarbamoyl)-1-(4-fluorobenzyl)-3-methyl-1H-pyrazol-4-yl]-2-methoxyquinoline-4-carboxamide,
6-fluoro-N⁴-[1-({5-[(2-hydroxyethyl)carbamoyl]pyridin-2-yl}methyl)-3,5-dimethyl-1H-pyrazol-4-yl]quinoline-2,4-dicarboxamide,
N⁴-(3,5-dimethyl-1-{[1-(methylcarbamoyl)piperidin-4-yl]methyl}-1H-pyrazol-4-yl)quinoline-2,4-dicarboxamide,
N-{3,5-dimethyl-1-[(3-methylpyridin-2-yl)methyl]-1H-pyrazol-4-yl}-2-methoxyquinoline-4-carboxamide,
N-[1-(4-fluorobenzyl)-3,5-dimethyl-1H-pyrazol-4-yl]-2,3-dihydro-1H-cyclopenta[b]quinoline-9-carboxamide,
N⁴-[5-(dimethylcarbamoyl)-1-(4-fluorobenzyl)-3-methyl-1H-pyrazol-4-yl]quinoline-2,4-dicarboxamide,
6-bromo-N-(1-{4-[(cyclohexylmethyl)carbamoyl]benzyl}-3,5-dimethyl-1H-pyrazol-4-yl)-2-(trifluoromethyl)quinoline-4-carboxamide,
N-[1-(4-fluorobenzyl)-3,5-dimethyl-1H-pyrazol-4-yl]-3-methylquinoline-4-carboxamide,
N-[1-(4-fluorobenzyl)-5-methyl-3-(methylcarbamoyl)-1H-pyrazol-4-yl]-2-methoxyquinoline-4-carboxamide,
6-bromo-N-{1-[4-(2-{[2-(dimethylamino)ethyl]amino}-2-oxoethyl)benzyl]-3,5-dimethyl-1H-pyrazol-4-yl}-2-(trifluoromethyl)quinoline-4-carboxamide,
ethyl 4-({[6-bromo-2-(trifluoromethyl)quinolin-4-yl]carbonyl}amino)-1-(4-fluorobenzyl)-3-methyl-1H-pyrazole-5-carboxylate,
6-bromo-N-[1-(3,5-difluorobenzyl)-3,5-dimethyl-1H-pyrazol-4-yl]-2-(trifluoromethyl)quinoline-4-carboxamide,
N-[1-(4-fluorobenzyl)-3,5-dimethyl-1H-pyrazol-4-yl]-6-[(methylsulfonyl)amino]-2-(trifluoromethyl)quinoline-4-carboxamide,
N⁴-[1-(4-fluorobenzyl)-3-methyl-5-(methylcarbamoyl)-1H-pyrazol-4-yl]quinoline-2,4-dicarboxamide,
6-bromo-N-[3-(dimethylcarbamoyl)-1-(4-fluorobenzyl)-5-methyl-1H-pyrazol-4-yl]-2-(trifluoromethyl)quinoline-4-carboxamide,
N-[1-(4-fluorobenzyl)-3,5-dimethyl-1H-pyrazol-4-yl]-2-hydroxyquinoline-4-carboxamide,
N-[3,5-dimethyl-1-(pyridin-2-ylmethyl)-1H-pyrazol-4-yl]-2,6-dimethylquinoline-4-carboxamide,
N-[3,5-dimethyl-1-(tetrahydro-2H-pyran-4-ylmethyl)-1H-pyrazol-4-yl]-6,7-difluoro-2-(trifluoromethyl)quinoline-4-carboxamide,
6-bromo-N-[1-({5-[(2,3-dihydroxypropyl)carbamoyl]pyridin-2-yl}methyl)-3,5-dimethyl-1H-pyrazol-4-yl]-2-(trifluoromethyl)quinoline-4-carboxamide,
3-cyano-N-[1-(4-fluorobenzyl)-3,5-dimethyl-1H-pyrazol-4-yl]-2-methylquinoline-4-carboxamide,
N-[3-(dimethylcarbamoyl)-1-(4-fluorobenzyl)-5-methyl-1H-pyrazol-4-yl]-2-methoxyquinoline-4-carboxamide,
6-bromo-N-(3,5-dimethyl-1-{[(2S)-5-oxopyrrolidin-2-yl]methyl}-1H-pyrazol-4-yl)-2-(trifluoromethyl)quinoline-4-carboxamide,
4-{[1-(4-fluorobenzyl)-3,5-dimethyl-1H-pyrazol-4-yl]carbamoyl}-2-(trifluoromethyl)quinoline-6-carboxylic acid,
8-cyano-N-[1-(4-fluorobenzyl)-3,5-dimethyl-1H-pyrazol-4-yl]-2-(trifluoromethyl)quinoline-4-carboxamide,
N-{3,5-dimethyl-1-[4-(trifluoromethyl)benzyl]-1H-pyrazol-4-yl}-2-methoxyquinoline-4-carboxamide,
6,7-difluoro-N-[1-(2-methoxybenzyl)-3,5-dimethyl-1H-pyrazol-4-yl]-2-(trifluoromethyl)quinoline-4-carboxamide,
4-({[6-bromo-2-(trifluoromethyl)quinolin-4-yl]carbonyl}amino)-1-(4-fluorobenzyl)-5-methyl-1H-pyrazole-3-carboxylic acid,
N-[1-(4-fluorobenzyl)-3,5-dimethyl-1H-pyrazol-4-yl]-2-hydroxy-3-methylquinoline-4-carboxamide,
N-{1-[(3,5-dimethyl-1,2-oxazol-4-yl)methyl]-3,5-dimethyl-1H-pyrazol-4-yl}-2-methoxyquinoline-4-carboxamide,
6-bromo-N-[1-(2-methoxybenzyl)-3,5-dimethyl-1H-pyrazol-4-yl]-2-(trifluoromethyl)quinoline-4-carboxamide,
6-[(4-{[(2-carbamoyl-6-fluoroquinolin-4-yl)carbonyl]amino}-3,5-dimethyl-1H-pyrazol-1-yl)methyl]nicotinic acid,
N-[1-(4-fluorobenzyl)-3,5-dimethyl-1H-pyrazol-4-yl]-2,7-bis(trifluoromethyl)quinoline-4-carboxamide,
7-bromo-N-[1-(4-fluorobenzyl)-3,5-dimethyl-1H-pyrazol-4-yl]-2-methylquinoline-4-carboxamide,
N-[1-(4-fluorobenzyl)-3,5-dimethyl-1H-pyrazol-4-yl]-2,7-dimethylquinoline-4-carboxamide,
6-bromo-N-{3,5-dimethyl-1-[2-(4-methylpiperazin-1-yl)ethyl]-1H-pyrazol-4-yl}-2-(trifluoromethyl)quinoline-4-carboxamide,
N-{3,5-dimethyl-1-[(5,5,8,8-tetramethyl-5,6,7,8-tetrahydronaphthalen-2-yl)methyl]-1H-pyrazol-4-yl}-2,6-dimethylquinoline-4-carboxamide,
N-[1-(2,5-difluorobenzyl)-3,5-dimethyl-1H-pyrazol-4-yl]-2,6-dimethylquinoline-4-carboxamide,
6-bromo-N-{1-[2-(3-fluorophenyl)ethyl]-3,5-dimethyl-1H-pyrazol-4-yl}-2-(trifluoromethyl)quinoline-4-carboxamide,
N-[1-(3-cyanobenzyl)-3,5-dimethyl-1H-pyrazol-4-yl]-2,6-dimethylquinoline-4-carboxamide,
N-[1-(3,5-difluorobenzyl)-3,5-dimethyl-1H-pyrazol-4-yl]-2-methoxyquinoline-4-carboxamide,
N-{3,5-dimethyl-1-[(3-methylpyridin-2-yl)methyl]-1H-pyrazol-4-yl}-2, 6-dimethylquinoline-4-carboxamide,
N-[1-(3-cyanobenzyl)-3,5-dimethyl-1H-pyrazol-4-yl]-2-methoxyquinoline-4-carboxamide,
N-{3,5-dimethyl-1-[2-(trifluoromethoxy)benzyl]-1H-pyrazol-4-yl}-2,6-dimethylquinoline-4-carboxamide,
6,8-dichloro-N-{3,5-dimethyl-1-[2-(trifluoromethoxy)benzyl]-1H-pyrazol-4-yl}-2-(trifluoromethyl)quinoline-4-carboxamide,
N-{3,5-dimethyl-1-[2-(trifluoromethoxy)benzyl]-1H-pyrazol-4-yl}-2-methoxyquinoline-4-carboxamide,
N-[1-(4-tert-butylbenzyl)-3,5-dimethyl-1H-pyrazol-4-yl]-2,6-dimethylquinoline-4-carboxamide,
N-{3,5-dimethyl-1-[2-(morpholin-4-yl)ethyl]-1H-pyrazol-4-yl}-2-methoxyquinoline-4-carboxamide,
6-bromo-N-{1-[(5-{[2-(dimethylamino)ethyl]carbamoyl}pyridin-2-yl)methyl]-3,5-dimethyl-1H-pyrazol-4-yl}-2-(trifluoromethyl)quinoline-4-carboxamide,
N-[1-(3,5-dimethoxybenzyl)-3,5-dimethyl-1H-pyrazol-4-yl]-2,6-dimethylquinoline-4-carboxamide,
N⁴-[1-(4-fluorobenzyl)-3,5-dimethyl-1H-pyrazol-4-yl]-2-(trifluoromethyl)quinoline-4, 6-dicarboxamide,
6,8-dichloro-N-[1-(2,5-difluorobenzyl)-3,5-dimethyl-1H-pyrazol-4-yl]-2-(trifluoromethyl)quinoline-4-carboxamide,
N-[1-(3,5-difluorobenzyl)-3,5-dimethyl-1H-pyrazol-4-yl]-2,6-dimethylquinoline-4-carboxamide,
6-bromo-N-{3,5-dimethyl-1-[3-(trifluoromethyl) benzyl]-1H-pyrazol-4-yl}-2-(trifluoromethyl)quinoline-4-carboxamide,
N-{3,5-dimethyl-1-[2-(trifluoromethyl)benzyl]-1H-pyrazol-4-yl}-2,6-dimethylquinoline-4-carboxamide,
6,8-dichloro-N-{3,5-dimethyl-1-[2-(trifluoromethyl)benzyl]-1H-pyrazol-4-yl}-2-(trifluoromethyl)quinoline-4-carboxamide,
N-{3,5-dimethyl-1-[3-(trifluoromethyl)benzyl]-1H-pyrazol-4-yl}-2,6-dimethylquinoline-4-carboxamide,
6-bromo-N-{3,5-dimethyl-1-[2-(trifluoromethyl)benzyl]-1H-pyrazol-4-yl}-2-(trifluoromethyl)quinoline-4-carboxamide,
6-bromo-N-{3,5-dimethyl-1-[4-(trifluoromethyl) benzyl]-1H-pyrazol-4-yl}-2-(trifluoromethyl)quinoline-4-carboxamide,
N-{3,5-dimethyl-1-[3-(trifluoromethyl)benzyl]-1H-pyrazol-4-yl}-2-methoxyquinoline-4-carboxamide,
N-[1-(2,5-difluorobenzyl)-3,5-dimethyl-1H-pyrazol-4-yl]-2-methoxyquinoline-4-carboxamide,
N-{3,5-dimethyl-1-[2-(trifluoromethyl)benzyl]-1H-pyrazol-4-yl}-2-methoxyquinoline-4-carboxamide,
6,8-dichloro-N-{3,5-dimethyl-1-[3-(trifluoromethyl)benzyl]-1H-pyrazol-4-yl}-2-(trifluoromethyl)quinoline-4-carboxamide,
N-[1-(4-fluorobenzyl)-3,5-dimethyl-1H-pyrazol-4-yl]-2,8-bis(trifluoromethyl)quinoline-4-carboxamide,
8-cyano-N-[1-(4-fluorobenzyl)-3,5-dimethyl-1H-pyrazol-4-yl]-2-methylquinoline-4-carboxamide,
7-cyano-N-[1-(4-fluorobenzyl)-3,5-dimethyl-1H-pyrazol-4-yl]-2-methylquinoline-4-carboxamide,
3-cyano-N-[1-(4-fluorobenzyl)-3,5-dimethyl-1H-pyrazol-4-yl]-2-hydroxyquinoline-4-carboxamide,
6-bromo-N-[1-(2-cyanobenzyl)-3,5-dimethyl-1H-pyrazol-4-yl]-2-cyclopropylquinoline-4-carboxamide,
N-[3,5-dimethyl-1-(2-naphthylmethyl)-1H-pyrazol-4-yl]-2-methoxyquinoline-4-carboxamide,
N-[1-(2-chlorobenzyl)-3,5-dimethyl-1H-pyrazol-4-yl]-2-methoxyquinoline-4-carboxamide,
N-[1-(biphenyl-2-ylmethyl)-3,5-dimethyl-1H-pyrazol-4-yl]-2-methoxyquinoline-4-carboxamide,
N-[1-(biphenyl-4-ylmethyl)-3,5-dimethyl-1H-pyrazol-4-yl]-2-methoxyquinoline-4-carboxamide,
N-[1-(2,4-dichlorobenzyl)-3,5-dimethyl-1H-pyrazol-4-yl]-2-methoxyquinoline-4-carboxamide,
N-[1-(2-bromobenzyl)-3,5-dimethyl-1H-pyrazol-4-yl]-2-methoxyquinoline-4-carboxamide,
N-[3,5-dimethyl-1-(quinolin-8-ylmethyl)-1H-pyrazol-4-yl]-2,6-dimethylquinoline-4-carboxamide,
N-[1-(2,6-dichlorobenzyl)-3,5-dimethyl-1H-pyrazol-4-yl]-2,6-dimethylquinoline-4-carboxamide,
N-{1-[4-fluoro-2-(trifluoromethyl)benzyl]-3,5-dimethyl-1H-pyrazol-4-yl}-2,6-dimethylquinoline-4-carboxamide,
N-[3,5-dimethyl-1-(quinolin-8-ylmethyl)-1H-pyrazol-4-yl]-2-methoxyquinoline-4-carboxamide,
N-[1-(4-tert-butylbenzyl)-3,5-dimethyl-1H-pyrazol-4-yl]-2-methoxyquinoline-4-carboxamide,
N-[1-(3-chloro-2-fluorobenzyl)-3,5-dimethyl-1H-pyrazol-4-yl]-2-methoxyquinoline-4-carboxamide,
N-[1-(4-bromobenzyl)-3,5-dimethyl-1H-pyrazol-4-yl]-2-methoxyquinoline-4-carboxamide,
N-[1-(diphenylmethyl)-3,5-dimethyl-1H-pyrazol-4-yl]-2-methoxyquinoline-4-carboxamide,
N-[1-(3,5-dimethylbenzyl)-3,5-dimethyl-1H-pyrazol-4-yl]-2-methoxyquinoline-4-carboxamide,
N-[3,5-dimethyl-1-(quinolin-2-ylmethyl)-1H-pyrazol-4-yl]-2-methoxyquinoline-4-carboxamide,
N-{1-[(3,5-dimethyl-1,2-oxazol-4-yl)methyl]-3,5-dimethyl-1H-pyrazol-4-yl}-2,6-dimethylquinoline-4-carboxamide,
N-[1-(3,4-dichlorobenzyl)-3,5-dimethyl-1H-pyrazol-4-yl]-2-methoxyquinoline-4-carboxamide,
N-[3,5-dimethyl-1-(pyridin-4-ylmethyl)-1H-pyrazol-4-yl]-2,6-dimethylquinoline-4-carboxamide,
N-{1-[3-fluoro-5-(trifluoromethyl)benzyl]-3,5-dimethyl-1H-pyrazol-4-yl}-2,6-dimethylquinoline-4-carboxamide,
N-[1-(2,1,3-benzothiadiazol-4-ylmethyl)-3,5-dimethyl-1H-pyrazol-4-yl]-2-methoxyquinoline-4-carboxamide,
N-{3,5-dimethyl-1-[(5,5,8,8-tetramethyl-5,6,7,8-tetrahydronaphthalen-2-yl)methyl]-1H-pyrazol-4-yl}-2-methoxyquinoline-4-carboxamide,
N-{1-[2-(1H-indol-3-yl)ethyl]-3,5-dimethyl-1H-pyrazol-4-yl}-2-methoxyquinoline-4-carboxamide,
N-{3,5-dimethyl-1-[2-(1H-pyrrol-1-yl)ethyl]-1H-pyrazol-4-yl}-2-methoxyquinoline-4-carboxamide,
N-[1-(2-chloro-4-fluorobenzyl)-3,5-dimethyl-1H-pyrazol-4-yl]-2-methoxyquinoline-4-carboxamide,
N-[1-(3,5-dimethoxybenzyl)-3,5-dimethyl-1H-pyrazol-4-yl]-2-methoxyquinoline-4-carboxamide,
6-bromo-N-[3-cyano-1-(4-fluorobenzyl)-5-methyl-1H-pyrazol-4-yl]-2-(trifluoromethyl)quinoline-4-carboxamide,
N⁴-[3,5-dimethyl-1-(1,3-thiazol-4-ylmethyl)-1H-pyrazol-4-yl]quinoline-2,4-dicarboxamide,
2-methoxy-N-{1-[2-(4-methoxyphenyl)ethyl]-3,5-dimethyl-1H-pyrazol-4-yl}quinoline-4-carboxamide,
6-bromo-N-{1-[(1,5-dimethyl-1H-pyrazol-4-yl)methyl]-3,5-dimethyl-1H-pyrazol-4-yl}-2-(trifluoromethyl)quinoline-4-carboxamide,
6-bromo-N-{1-[2-(4-cyanophenoxy)ethyl]-3,5-dimethyl-1H-pyrazol-4-yl}-2-(trifluoromethyl)quinoline-4-carboxamide,
6-chloro-7-fluoro-N⁴-{1-[2-(4-methoxyphenyl)ethyl]-3,5-dimethyl-1H-pyrazol-4-yl}quinoline-2,4-dicarboxamide,
N⁴-{3,5-dimethyl-1-[(2-oxo-1,3-oxazolidin-5-yl)methyl]-1H-pyrazol-4-yl}quinoline-2,4-dicarboxamide,
N-{1-[2-(4-cyanophenoxy)ethyl]-3,5-dimethyl-1H-pyrazol-4-yl}-2-methoxyquinoline-4-carboxamide,
6-bromo-N-[1-(4-cyano-2-fluorobenzyl)-3,5-dimethyl-1H-pyrazol-4-yl]-2-(trifluoromethyl)quinoline-4-carboxamide,
6-bromo-N-{1-[2-(4-methoxyphenyl)ethyl]-3,5-dimethyl-1H-pyrazol-4-yl}-2-(trifluoromethyl)quinoline-4-carboxamide,
6-bromo-N-[3,5-dimethyl-1-(1,3-thiazol-4-ylmethyl)-1H-pyrazol-4-yl]-2-(trifluoromethyl)quinoline-4-carboxamide,
N-[1-(4-cyano-2-fluorobenzyl)-3,5-dimethyl-1H-pyrazol-4-yl]-2-methoxyquinoline-4-carboxamide,
N⁴{1-[2-(4-methoxyphenyl)ethyl]-3,5-dimethyl-1H-pyrazol-4-yl}quinoline-2,4-dicarboxamide,
6-bromo-N-{3,5-dimethyl-1-[(2-methyl-1,3-thiazol-4-yl)methyl]-1H-pyrazol-4-yl}-2-(trifluoromethyl)quinoline-4-carboxamide,
6-bromo-N-[1-(4-fluorobenzyl)-3,5-di(propan-2-yl)-1H-pyrazol-4-yl]-2-(trifluoromethyl)quinoline-4-carboxamide,
N-[1-(4-fluorobenzyl)-3,5-di(propan-2-yl)-1H-pyrazol-4-yl]-2,6-dimethylquinoline-4-carboxamide,
6,7-difluoro-N-[1-(4-fluorobenzyl)-3,5-di(propan-2-yl)-1H-pyrazol-4-yl]-2-(trifluoromethyl)quinoline-4-carboxamide,
6-bromo-N-[1-(4-cyano-2-fluorobenzyl)-5-methyl-3-(trifluoromethyl)-1H-pyrazol-4-yl]-2-(trifluoromethyl)quinoline-4-carboxamide,
N-[1-(4-cyano-2-fluorobenzyl)-5-methyl-3-(trifluoromethyl)-1H-pyrazol-4-yl]-2-methoxyquinoline-4-carboxamide,
(±)-6-bromo-N-[3,5-dimethyl-1-(1-phenylethyl)-1H-pyrazol-4-yl]-2-(trifluoromethyl)quinoline-4-carboxamide,
(±)-N⁴-[3,5-dimethyl-1-(1-phenylethyl)-1H-pyrazol-4-yl]-2-methoxyquinoline-4-carboxamide,
(±)-N⁴-[3,5-dimethyl-1-(1-phenylethyl)-1H-pyrazol-4-yl]quinoline-2,4-dicarboxamide,
(±)-6-chloro-N⁴-[3,5-dimethyl-1-(1-phenylethyl)-1H-pyrazol-4-yl]-7-fluoroquinoline-2,4-dicarboxamide,
(±)-N⁴-{1-[1-(4-fluorophenyl)ethyl]-3,5-dimethyl-1H-pyrazol-4-yl}quinoline-2,4-dicarboxamide,
6-bromo-N-[5-methyl-1-(pyridin-4-ylmethyl)-3-(trifluoromethyl)-1H-pyrazol-4-yl]-2-(trifluoromethyl)quinoline-4-carboxamide,
2-methoxy-N-[5-methyl-1-(pyridin-4-ylmethyl)-3-(trifluoromethyl)-1H-pyrazol-4-yl]quinoline-4-carboxamide,
N⁴-[5-methyl-1-(pyridin-4-ylmethyl)-3-(trifluoromethyl)-1H-pyrazol-4-yl]quinoline-2,4-dicarboxamide,
6-chloro-7-fluoro-N⁴-[5-methyl-1-(pyridin-4-ylmethyl)-3-(trifluoromethyl)-1H-pyrazol-4-yl]quinoline-2,4-dicarboxamide,
N⁴-[1-(4-tert-butylbenzyl)-3,5-dimethyl-1H-pyrazol-4-yl]quinoline-2,4-dicarboxamide,
N⁴-[1-(4-tert-butylbenzyl)-5-methyl-3-(trifluoromethyl)-1H-pyrazol-4-yl]quinoline-2,4-dicarboxamide,
N⁴-{1-[2-(4-fluorophenyl)ethyl]-5-methyl-3-(trifluoromethyl)-1H-pyrazol-4-yl}quinoline-2,4-dicarboxamide,
N-{1-[2-(4-fluorophenyl)ethyl]-5-methyl-3-(trifluoromethyl)-1H-pyrazol-4-yl}-2-methoxyquinoline-4-carboxamide,
6-bromo-N-{1-[2-(4-fluorophenyl)ethyl]-5-methyl-3-(trifluoromethyl)-1H-pyrazol-4-yl}-2-(trifluoromethyl)quinoline-4-carboxamide,
(±)-6-bromo-N-{1-[1-(4-fluorophenyl)ethyl]-3,5-dimethyl-1H-pyrazol-4-yl}-2-(trifluoromethyl)quinoline-4-carboxamide,
N-{1-[1-(4-fluorophenyl)ethyl]-3,5-dimethyl-1H-pyrazol-4-yl}-2-methoxyquinoline-4-carboxamide,
N-[1-(4-cyanobenzyl)-5-methyl-3-(trifluoromethyl)-1H-pyrazol-4-yl]-6-fluoro-2-methoxyquinoline-4-carboxamide,
N⁴-[1-(4-fluorobenzyl)-3,5-dimethyl-1H-pyrazol-4-yl]-6-methylquinoline-2,4-dicarboxamide,
(±)-6-bromo-N-{1-[4-(4-fluorophenyl)butan-2-yl]-3,5-dimethyl-1H-pyrazol-4-yl}-2-(trifluoromethyl)quinoline-4-carboxamide,
(±)-N⁴-{1-[4-(4-fluorophenyl)butan-2-yl]-3,5-dimethyl-1H-pyrazol-4-yl}quinoline-2,4-dicarboxamide,
(±)-6-chloro-7-fluoro-N⁴-{1-[4-(4-fluorophenyl)butan-2-yl]-3,5-dimethyl-1H-pyrazol-4-yl}quinoline-2,4-dicarboxamide,
N⁴-[1-(4-tert-butylbenzyl)-5-methyl-3-(trifluoromethyl)-1H-pyrazol-4-yl]-7-fluoroquinoline-2,4-dicarboxamide,
N⁴-[1-(4-cyanobenzyl)-5-methyl-3-(trifluoromethyl)-1H-pyrazol-4-yl]-6-sulfamoylquinoline-2,4-dicarboxamide,
N⁴-[1-(4-tert-butylbenzyl)-3,5-dimethyl-1H-pyrazol-4-yl]-7-fluoroquinoline-2,4-dicarboxamide,
N⁴-[3,5-diethyl-1-(4-fluorobenzyl)-1H-pyrazol-4-yl]-8-fluoroquinoline-2,4-dicarboxamide,
N⁴-[3,5-diethyl-1-(4-fluorobenzyl)-1H-pyrazol-4-yl]-7-fluoroquinoline-2,4-dicarboxamide,
6-chloro-N⁴-[3,5-diethyl-1-(4-fluorobenzyl)-1H-pyrazol-4-yl]-7-fluoroquinoline-2,4-dicarboxamide,
N⁴-[3,5-diethyl-1-(4-fluorobenzyl)-1H-pyrazol-4-yl]quinoline-2,4-dicarboxamide,
6-bromo-N-[3,5-diethyl-1-(4-fluorobenzyl)-1H-pyrazol-4-yl]-2-(trifluoromethyl)quinoline-4-carboxamide,
N⁴-[1-(4-cyanobenzyl)-5-methyl-3-(trifluoromethyl)-1H-pyrazol-4-yl]-6-(methylsulfonyl)quinoline-2,4-dicarboxamide,
methyl 4-{[1-(4-cyanobenzyl)-3,5-dimethyl-1H-pyrazol-4-yl]carbamoyl}-7-methoxyquinoline-2-carboxylate,
7-chloro-N⁴-[3,5-diethyl-1-(4-fluorobenzyl)-1H-pyrazol-4-yl]quinoline-2,4-dicarboxamide,
N⁴-{1-[(3-cyanopyridin-2-yl)methyl]-5-methyl-3-(trifluoromethyl)-1H-pyrazol-4-yl}-7-fluoroquinoline-2,4-dicarboxamide,
N⁴-{1-[(3-cyanopyridin-2-yl)methyl]-5-methyl-3-(trifluoromethyl)-1H-pyrazol-4-yl}quinoline-2,4-dicarboxamide,
N⁴-[1-(4-cyanobenzyl)-5-methyl-3-(trifluoromethyl)-1H-pyrazol-4-yl]-6-(4-methylpiperazin-1-yl)quinoline-2,4-dicarboxamide,
N⁴-{1-[(3-cyanopyridin-2-yl)methyl]-5-methyl-3-(trifluoromethyl)-1H-pyrazol-4-yl}-5-fluoroquinoline-2,4-dicarboxamide,
N⁴-[1-(3,4-difluorobenzyl)-5-methyl-3-(trifluoromethyl)-1H-pyrazol-4-yl]-7-fluoroquinoline-2,4-dicarboxamide,
(±)-N⁴-[5-methyl-1-(1-phenylethyl)-3-(trifluoromethyl)-1H-pyrazol-4-yl]quinoline-2,4-dicarboxamide,
(±)-6-bromo-N-[5-methyl-1-(1-phenylethyl)-3-(trifluoromethyl)-1H-pyrazol-4-yl]-2-(trifluoromethyl)quinoline-4-carboxamide,
N⁴-[1-(4-cyanobenzyl)-3,5-dimethyl-1H-pyrazol-4-yl]-7-fluoro-N²-(2-hydroxyethyl)quinoline-2,4-dicarboxamide,
N⁴-[1-(4-cyanobenzyl)-5-methyl-3-(trifluoromethyl)-1H-pyrazol-4-yl]-N²-[2-(dimethylamino)ethyl]-7-fluoroquinoline-2,4-dicarboxamide,
N⁴-[1-(4-cyanobenzyl)-3,5-dimethyl-1H-pyrazol-4-yl]-N²-[2-(dimethylamino)ethyl]-7-fluoroquinoline-2,4-dicarboxamide,
N⁴-[1-(4-cyanobenzyl)-3,5-dimethyl-1H H-pyrazol-4-yl]-7-fluoro-N<sup>2</sup>-[2-(morpholin-4-yl)ethyl]quinoline-2,4-dicarboxamide,
N⁴-[1-(4-cyanobenzyl)-3,5-dimethyl-1H H-pyrazol-4-yl]-7-fluoro-N²-[2-(morpholin-4-yl)ethyl]quinoline-2,4-dicarboxamide,
N⁴-[1-(4-cyanobenzyl)-5-methyl-3-(trifluoromethyl)-1H-pyrazol-4-yl]-7-fluoro-N²-(2-hydroxyethyl)quinoline-2,4-dicarboxamide,
7-fluoro-N⁴-[1-(4-isopropylbenzyl)-3,5-dimethyl-1H-pyrazol-4-yl]quinoline-2,4-dicarboxamide,
N⁴-[1-(4-isopropylbenzyl)-3,5-dimethyl-1H-pyrazol-4-yl]quinoline-2,4-dicarboxamide,
7-fluoro-N⁴-[1-(4-isopropylbenzyl)-5-methyl-3-(trifluoromethyl)-1H-pyrazol-4-yl]quinoline-2,4-dicarboxamide,
N⁴-[1-(4-isopropylbenzyl)-5-methyl-3-(trifluoromethyl)-1H-pyrazol-4-yl]quinoline-2,4-dicarboxamide,
6-bromo-N⁴-{1-[(3-cyanopyridin-2-yl)methyl]-3,5-dimethyl-1H-pyrazol-4-yl}quinoline-2,4-dicarboxamide,
N⁴-{1-[(3-cyanopyridin-2-yl)methyl]-3,5-dimethyl-1H-pyrazol-4-yl}-7-fluoroquinoline-2,4-dicarboxamide,
N⁴-{1[(3-cyanopyridin-2-yl)methyl]-3,5-dimethyl-1H-pyrazol-4-yl}-5-fluoroquinoline-2,4-dicarboxamide,
6-chloro-N⁴-{1-[(3-cyanopyridin-2-yl)methyl]-3,5-dimethyl-1H-pyrazol-4-yl}-7-fluoroquinoline-2,4-dicarboxamide,
N⁴-[1-(4-ethylbenzyl)-5-methyl-3-(trifluoromethyl)-1H-pyrazol-4-yl]-7-fluoroquinoline-2,4-dicarboxamide,
N⁴-[1-(4-ethylbenzyl)-5-methyl-3-(trifluoromethyl)-1H-pyrazol-4-yl]quinoline-2,4-dicarboxamide,
6-bromo-N⁴-[1-(4-ethylbenzyl)-5-methyl-3-(trifluoromethyl)-1H-pyrazol-4-yl]quinoline-2,4-dicarboxamide,
6-chloro-N⁴-[1-(4-ethylbenzyl)-5-methyl-3-(trifluoromethyl)-1H-pyrazol-4-yl]-7-fluoroquinoline-2,4-dicarboxamide,
6-bromo-N-[3,5-dimethyl-1-(pyrimidin-4-ylmethyl)-1H-pyrazol-4-yl]-2-(trifluoromethyl)quinoline-4-carboxamide,
N⁴-[3,5-dimethyl-1-(pyrimidin-4-ylmethyl)-1H-pyrazol-4-yl]quinoline-2,4-dicarboxamide,
N-[3-cyano-1-(4-fluorobenzyl)-5-methyl-1H-pyrazol-4-yl]-2-methoxyquinoline-4-carboxamide,
N⁴-{1-[4-(azetidin-1-ylcarbonyl)benzyl]-3,5-dimethyl-1H-pyrazol-4-yl}-7-fluoroquinoline-2,4-dicarboxamide,
7-fluoro-N⁴-(1-{4-[(2-hydroxyethyl)carbamoyl]benzyl}-3,5-dimethyl-1H-pyrazol-4-yl)quinoline-2,4-dicarboxamide,
7-fluoro-N⁴-(1-{4-[(2-methoxyethyl)carbamoyl]benzyl}-3,5-dimethyl-1H-pyrazol-4-yl)quinoline-2,4-dicarboxamide,
N⁴-[1-(4-{[2-(dimethylamino)ethyl]carbamoyl}benzyl)-3,5-dimethyl-1H-pyrazol-4-yl]-7-fluoroquinoline-2,4-dicarboxamide,
4-[(4-{[(2-carbamoyl-7-fluoroquinolin-4-yl)carbonyl]amino}-3,5-dimethyl-1H-pyrazol-1-yl)methyl]benzoic acid,
N⁴-[1-(4-carbamoylbenzyl)-3,5-dimethyl-1H-pyrazol-4-yl]-7-fluoroquinoline-2,4-dicarboxamide,
N⁴-{3,5-dimethyl-1-[4-(methylcarbamoyl)benzyl]-1H-pyrazol-4-yl}-7-fluoroquinoline-2,4-dicarboxamide,
N⁴-{1-[4-(dimethylcarbamoyl)benzyl]-3,5-dimethyl-1H-pyrazol-4-yl}-7-fluoroquinoline-2,4-dicarboxamide,
(±_)-6-bromo-N-{3,5-dimethyl-1-[4-(S-methylsulfonimidoyl)benzyl]-1H-pyrazol-4-yl}-2-(trifluoromethyl)quinoline-4-carboxamide,
2-(azetidin-1-ylcarbonyl)-N-[1-(4-cyanobenzyl)-3,5-dimethyl-1H-pyrazol-4-yl]-7-fluoroquinoline-4-carboxamide,
7-fluoro-N⁴-{5-methyl-1-[4-(methylsulfonyl)benzyl]-3-(trifluoromethyl)-1H-pyrazol-4-yl}quinoline-2,4-dicarboxamide,
6-bromo-N-{5-methyl-1-[4-(methylsulfonyl)benzyl]-3-(trifluoromethyl)-1H-pyrazol-4-yl}-2-(trifluoromethyl)quinoline-4-carboxamide,
2-methoxy-N-{5-methyl-1-[4-(methylsulfonyl)benzyl]-3-(trifluoromethyl)-1H-pyrazol-4-yl}quinoline-4-carboxamide,
N⁴-{5-methyl-1-[4-(methylsulfonyl)benzyl]-3-(trifluoromethyl)-1H-pyrazol-4-yl}quinoline-2,4-dicarboxamide,
(±)-ethyl [(4-{[4-({[6-bromo-2-(trifluoromethyl)quinolin-4-yl]carbonyl}amino)-3,5-dimethyl-1H-pyrazol-1-yl]methyl}phenyl)(methyl)oxido-λ⁶-sulfanylidene]carbamate,
N⁴-[1-(4-cyanobenzyl)-5-methyl-3-(trifluoromethyl)-1H-pyrazol-4-yl]-6-{[2-(dimethylamino)ethyl]amino}quinoline-2,4-dicarboxamide,
N⁴-[1-(2,4-difluorobenzyl)-5-methyl-3-(trifluoromethyl)-1H-pyrazol-4-yl]-7-fluoroquinoline-2,4-dicarboxamide,
N-[1-(4-fluorobenzyl)-5-methyl-3-(trifluoromethyl)-1H-pyrazol-4-yl]-2-methoxyquinoline-4-carboxamide,
5-fluoro-N⁴-{5-methyl-1-[4-(methylsulfonyl)benzyl]-3-(trifluoromethyl)-1H-pyrazol-4-yl}quinoline-2,4-dicarboxamide,
6-chloro-7-fluoro-N⁴-{5-methyl-1-[4-(methylsulfonyl)benzyl]-3-(trifluoromethyl)-1H-pyrazol-4-yl}quinoline-2,4-dicarboxamide,
6-bromo-N⁴-{5-methyl-1-[4-(methylsulfonyl)benzyl]-3-(trifluoromethyl)-1H-pyrazol-4-yl}quinoline-2,4-dicarboxamide,
N⁴-[1-(2,4-difluorobenzyl)-5-methyl-3-(trifluoromethyl)-1H-pyrazol-4-yl]-5-fluoroquinoline-2,4-dicarboxamide,
6-chloro-N⁴-[1-(2,4-difluorobenzyl)-5-methyl-3-(trifluoromethyl)-1H-pyrazol-4-yl]-7-fluoroquinoline-2,4-dicarboxamide,
6-bromo-N⁴-[1-(2,4-difluorobenzyl)-5-methyl-3-(trifluoromethyl)-1H-pyrazol-4-yl]quinoline-2,4-dicarboxamide,
N⁴-[1-(3,4-difluorobenzyl)-5-methyl-3-(trifluoromethyl)-1H-pyrazol-4-yl]-5-fluoroquinoline-2,4-dicarboxamide,
6-chloro-N⁴-[1-(3,4-difluorobenzyl)-5-methyl-3-(trifluoromethyl)-1H-pyrazol-4-yl]-7-fluoroquinoline-2,4-dicarboxamide,
6-bromo-N⁴-[1-(3,4-difluorobenzyl)-5-methyl-3-(trifluoromethyl)-1H-pyrazol-4-yl]quinoline-2,4-dicarboxamide,
6-chloro-7-fluoro-N⁴-[1-(3-methoxybenzyl)-5-methyl-3-(trifluoromethyl)-1H-pyrazol-4-yl]quinoline-2,4-dicarboxamide,
7-fluoro-N⁴-[1-(3-methoxybenzyl)-5-methyl-3-(trifluoromethyl)-1H-pyrazol-4-yl]quinoline-2,4-dicarboxamide,
N⁴-[1-(3-methoxybenzyl)-5-methyl-3-(trifluoromethyl)-1H-pyrazol-4-yl]quinoline-2,4-dicarboxamide,
5-fluoro-N⁴-[1-(3-methoxybenzyl)-5-methyl-3-(trifluoromethyl)-1H-pyrazol-4-yl]quinoline-2,4-dicarboxamide,
6-bromo-N⁴-[1-(3-methoxybenzyl)-5-methyl-3-(trifluoromethyl)-1H-pyrazol-4-yl]quinoline-2,4-dicarboxamide,
5-fluoro-N⁴-[1-(3-methoxybenzyl)-3,5-dimethyl-1H-pyrazol-4-yl]quinoline-2,4-dicarboxamide,
N⁴-[1-(3-methoxybenzyl)-3,5-dimethyl-1H-pyrazol-4-yl]quinoline-2,4-dicarboxamide,
6-chloro-7-fluoro-N⁴-[1-(3-methoxybenzyl)-3,5-dimethyl-1H-pyrazol-4-yl]quinoline-2,4-dicarboxamide,
N⁴-[1-(3-cyanobenzyl)-3,5-dimethyl-1H-pyrazol-4-yl]-7-fluoroquinoline-2,4-dicarboxamide,
N⁴-[1-(3-cyanobenzyl)-3,5-dimethyl-1H-pyrazol-4-yl]quinoline-2,4-dicarboxamide,
6-chloro-N⁴-[1-(3-cyanobenzyl)-3,5-dimethyl-1H-pyrazol-4-yl]-7-fluoroquinoline-2,4-dicarboxamide,
N⁴-[1-(3-cyanobenzyl)-3,5-dimethyl-1H-pyrazol-4-yl]-5-fluoroquinoline-2,4-dicarboxamide,
6-bromo-N⁴-[1-(3-methoxybenzyl)-3,5-dimethyl-1H-pyrazol-4-yl]quinoline-2,4-dicarboxamide,
7-fluoro-N⁴-[1-(3-methoxybenzyl)-3,5-dimethyl-1H-pyrazol-4-yl]quinoline-2,4-dicarboxamide,
N⁴-{3,5-dimethyl-1-[4-(trifluoromethoxy)benzyl]-1H-pyrazol-4-yl}quinoline-2,4-dicarboxamide,
N⁴-{3,5-dimethyl-1-[4-(trifluoromethoxy)benzyl]-1H-pyrazol-4-yl}-7-fluoroquinoline-2,4-dicarboxamide,
N⁴-{3,5-dimethyl-1-[4-(trifluoromethoxy)benzyl]-1H-pyrazol-4-yl}-5-fluoroquinoline-2,4-dicarboxamide,
6-chloro-N⁴-{3,5-dimethyl-1-[4-(trifluoromethoxy)benzyl]-1H-pyrazol-4-yl}-7-fluoroquinoline-2,4-dicarboxamide,
6-bromo-N⁴-{3,5-dimethyl-1-[4-(trifluoromethoxy)benzyl]-1H-pyrazol-4-yl}quinoline-2,4-dicarboxamide,
N⁴-{5-methyl-1-[4-(trifluoromethoxy)benzyl]-3-(trifluoromethyl)-1H-pyrazol-4-yl}quinoline-2,4-dicarboxamide,
7-fluoro-N⁴-{5-methyl-1-[4-(trifluoromethoxy)benzyl]-3-(trifluoromethyl)-1H-pyrazol-4-yl}quinoline-2,4-dicarboxamide,
5-fluoro-N⁴-{5-methyl-1-[4-(trifluoromethoxy)benzyl]-3-(trifluoromethyl)-1H-pyrazol-4-yl}quinoline-2,4-dicarboxamide,
6-chloro-7-fluoro-N⁴-{5-methyl-1-[4-(trifluoromethoxy)benzyl]-3-(trifluoromethyl)-1H-pyrazol-4-yl}quinoline-2,4-dicarboxamide,
6-bromo-N⁴-[1-(3,4-difluorobenzyl)-3,5-dimethyl-1H-pyrazol-4-yl]quinoline-2,4-dicarboxamide,
N⁴-[1-(3,4-difluorobenzyl)-3,5-dimethyl-1H-pyrazol-4-yl]-7-fluoroquinoline-2,4-dicarboxamide,
N⁴-[1-(3,4-difluorobenzyl)-3,5-dimethyl-1H-pyrazol-4-yl]-5-fluoroquinoline-2,4-dicarboxamide,
6-chloro-N⁴-[1-(3,4-difluorobenzyl)-3,5-dimethyl-1H-pyrazol-4-yl]-7-fluoroquinoline-2,4-dicarboxamide,
6-chloro-N⁴-[3,5-dimethyl-1-(3-methylbenzyl)-1H-pyrazol-4-yl]-7-fluoroquinoline-2,4-dicarboxamide,
N⁴-[3,5-dimethyl-1-(3-methylbenzyl)-1H-pyrazol-4-yl]-7-fluoroquinoline-2,4-dicarboxamide,
N⁴-[3,5-dimethyl-1-(3-methylbenzyl)-1H-pyrazol-4-yl]-5-fluoroquinoline-2,4-dicarboxamide,
6-chloro-N⁴-[3,5-dimethyl-1-(3-methylbenzyl)-1H-pyrazol-4-yl]-7-fluoroquinoline-2,4-dicarboxamide,
6-bromo-N⁴-[3,5-dimethyl-1-(3-methylbenzyl)-1H-pyrazol-4-yl]quinoline-2,4-dicarboxamide,
N⁴-[5-methyl-1-(3-methylbenzyl)-3-(trifluoromethyl)-1H-pyrazol-4-yl]quinoline-2,4-dicarboxamide,
5-fluoro-N⁴-[5-methyl-1-(3-methylbenzyl)-3-(trifluoromethyl)-1H-pyrazol-4-yl]quinoline-2,4-dicarboxamide,
7-fluoro-N⁴-[5-methyl-1-(3-methylbenzyl)-3-(trifluoromethyl)-1H-pyrazol-4-yl]quinoline-2,4-dicarboxamide,
6-chloro-7-fluoro-N⁴-[5-methyl-1-(3-methylbenzyl)-3-(trifluoromethyl)-1H-pyrazol-4-yl]quinoline-2,4-dicarboxamide,
6-bromo-N⁴-[5-methyl-1-(3-methylbenzyl)-3-(trifluoromethyl)-1H-pyrazol-4-yl]quinoline-2,4-dicarboxamide,
N⁴-{3,5-dimethyl-1-[4-(methylsulfonyl)benzyl]-1H-pyrazol-4-yl}-7-fluoroquinoline-2,4-dicarboxamide,
N⁴-{3,5-dimethyl-1-[4-(methylsulfonyl)benzyl]-1H-pyrazol-4-yl}-5-fluoroquinoline-2,4-dicarboxamide,
6-chloro-N⁴-{3,5-dimethyl-1-[4-(methylsulfonyl)benzyl]-1H-pyrazol-4-yl}-7-fluoroquinoline-2,4-dicarboxamide,
6-bromo-N⁴-{3,5-dimethyl-1-[4-(methylsulfonyl)benzyl]-1H-pyrazol-4-yl}quinoline-2,4-dicarboxamide,
N⁴-[1-(2,4-difluorobenzyl)-3,5-dimethyl-1H-pyrazol-4-yl]-7-fluoroquinoline-2,4-dicarboxamide,
N⁴-[1-(2,4-difluorobenzyl)-3,5-dimethyl-1H-pyrazol-4-yl]-5-fluoroquinoline-2,4-dicarboxamide,
6-chloro-N⁴-[1-(2,4-difluorobenzyl)-3,5-dimethyl-1H-pyrazol-4-yl]-7-fluoroquinoline-2,4-dicarboxamide,
6-bromo-N⁴-[1-(2,4-difluorobenzyl)-3,5-dimethyl-1H-pyrazol-4-yl]quinoline-2,4-dicarboxamide,
6-chloro-7-fluoro-N⁴-[1-(2-methoxybenzyl)-5-methyl-3-(trifluoromethyl)-1H-pyrazol-4-yl]quinoline-2,4-dicarboxamide,
N⁴-[1-(2-methoxybenzyl)-5-methyl-3-(trifluoromethyl)-1H-pyrazol-4-yl]quinoline-2,4-dicarboxamide,
5-fluoro-N⁴-[1-(2-methoxybenzyl)-5-methyl-3-(trifluoromethyl)-1H-pyrazol-4-yl]quinoline-2,4-dicarboxamide,
N⁴-[5-methyl-1-(2-methylbenzyl)-3-(trifluoromethyl)-1H-pyrazol-4-yl]quinoline-2,4-dicarboxamide,
7-fluoro-N⁴-[5-methyl-1-(2-methylbenzyl)-3-(trifluoromethyl)-1H-pyrazol-4-yl]quinoline-2,4-dicarboxamide,
5-fluoro-N⁴-[5-methyl-1-(2-methylbenzyl)-3-(trifluoromethyl)-1H-pyrazol-4-yl]quinoline-2,4-dicarboxamide,
6-chloro-7-fluoro-N⁴-[5-methyl-1-(2-methylbenzyl)-3-(trifluoromethyl)-1H-pyrazol-4-yl]quinoline-2,4-dicarboxamide,
6-bromo-N⁴-[5-methyl-1-[4-(trifluoromethoxy)benzyl]-3-(trifluoromethyl)-1H-pyrazol-4-yl]quinoline-2,4-dicarboxamide,
6-bromo-N-{1-[(5-ethyl-1,2,4-oxadiazol-3-yl)methyl]-3,5-dimethyl-1H-pyrazol-4-yl}-2-(trifluoromethyl)quinoline-4-carboxamide,
N⁴-{1-[(5-ethyl-1,2,4-oxadiazol-3-yl)methyl]-3,5-dimethyl-1H-pyrazol-4-yl}quinoline-2,4-dicarboxamide,
N⁴-{1-[(3-cyclopropyl-1,2,4-oxadiazol-5-yl)methyl]-3,5-dimethyl-1H-pyrazol-4-yl}quinoline-2,4-dicarboxamide,
6-bromo-N-{1-[(3-cyclopropyl-1,2,4-oxadiazol-5-yl)methyl]-3,5-dimethyl-1H-pyrazol-4-yl}-2-(trifluoromethyl)quinoline-4-carboxamide,
N⁴-{3,5-dimethyl-1-[(5-methyl-1,2-oxazol-3-yl)methyl]-1H-pyrazol-4-yl}quinoline-2,4-dicarboxamide,
N⁴-[3,5-dimethyl-1-(1,3-oxazol-2-ylmethyl)-1H-pyrazol-4-yl]quinoline-2,4-dicarboxamide,
6-bromo-N-(1-{[3-(methoxymethyl)-1,2,4-oxadiazol-5-yl]methyl}-3,5-dimethyl-1H-pyrazol-4-yl)-2-(trifluoromethyl)quinoline-4-carboxamide,
N⁴-(1-{[3-(methoxymethyl)-1, 2,4-oxadiazol-5-yl]methyl}-3,5-dimethyl-1H-pyrazol-4-yl)quinoline-2,4-dicarboxamide,
N⁴-(3,5-dimethyl-1-{[5-(methylcarbamoyl)-1,2,4-oxadiazol-3-yl]methyl}-1H-pyrazol-4-yl)quinoline-2,4-dicarboxamide,
N⁴-(3,5-dimethyl-1-{[1-(propylsulfonyl)piperidin-4-yl]methyl}-1H-pyrazol-4-yl)quinoline-2,4-dicarboxamide,
N⁴-(1-{[1-(butylsulfonyl)piperidin-4-yl]methyl}-3,5-dimethyl-1H-pyrazol-4-yl)quinoline-2,4-dicarboxamide,
N⁴-[1-({1-[(3-cyanopropyl)sulfonyl]piperidin-4-yl}methyl)-3,5-dimethyl-1H-pyrazol-4-yl]quinoline-2,4-dicarboxamide,
N⁴-[1-({1-[(3-methoxypropyl)sulfonyl]piperidin-4-yl}methyl)-3,5-dimethyl-1H-pyrazol-4-yl]quinoline-2,4-dicarboxamid,
N⁴-(3,5-dimethyl-1-{[1-(pyridin-3-ylsulfonyl)piperidin-4-yl]methyl}-1H-pyrazol-4-yl)quinoline-2,4-dicarboxamid,
N⁴-{3,5-dimethyl-1-[(4-methyl-1,2,5-oxadiazol-3-yl)methyl]-1H-pyrazol-4-yl}quinoline-2,4-dicarboxamide,
N⁴-(1-{[1-(cyclopropylsulfonyl)piperidin-4-yl]methyl}-3,5-dimethyl-1H-pyrazol-4-yl)quinoline-2,4-dicarboxamide,
N⁴-(1-{[1-(isopropylsulfonyl)piperidin-4-yl]methyl}-3,5-dimethyl-1H-pyrazol-4-yl)quinoline-2,4-dicarboxamide,
N⁴-(1-{[1-(cyclopentylsulfonyl)piperidin-4-yl]methyl}-3,5-dimethyl-1H-pyrazol-4-yl)quinoline-2,4-dicarboxamide,
N⁴-(3,5-dimethyl-1-{[1-(phenylsulfonyl)piperidin-4-yl]methyl}-1H-pyrazol-4-yl)quinoline-2,4-dicarboxamide,
6-bromo-N-{3,5-dimethyl-1-[(4-methyl-1,2,5-oxadiazol-3-yl)methyl]-1H-pyrazol-4-yl}-2-(trifluoromethyl)quinoline-4-carboxamide,
N⁴-(1-{[5-(dimethylcarbamoyl)-1,2,4-oxadiazol-3-yl]methyl}-3,5-dimethyl-1H-pyrazol-4-yl)quinoline-2,4-dicarboxamide,
6-bromo-N-(1-{[5-(dimethylcarbamoyl)-1,2,4-oxadiazol-3-yl]methyl}-3,5-dimethyl-1H-pyrazol-4-yl)-2-(trifluoromethyl)quinoline-4-carboxamide,
N⁴-{3,5-dimethyl-1-[(1-methyl-1H-imidazol-2-yl)methyl]-1H-pyrazol-4-yl}quinoline-2,4-dicarboxamide,
6-bromo-N-{3,5-dimethyl-1-[(1-methyl-1H-imidazol-2-yl)methyl]-1H-pyrazol-4-yl}-2-(trifluoromethyl)quinoline-4-carboxamide,
N-{3,5-dimethyl-1-[(2-oxo-1,3-oxazolidin-5-yl)methyl]-1H-pyrazol-4-yl}-2-methoxyquinoline-4-carboxamide,
N-{3,5-dimethyl-1-[(1-methyl-1H-pyrazol-3-yl)methyl]-1H-pyrazol-4-yl}-2-methoxyquinoline-4-carboxamide,
N-[1-(imidazo[1,2-a]pyridin-2-ylmethyl)-3,5-dimethyl-1H-pyrazol-4-yl]-2-methoxyquinoline-4-carboxamide,
6-bromo-N-[1-(imidazo[1,2-a]pyridin-2-ylmethyl)-3,5-dimethyl-1H-pyrazol-4-yl]-2-(trifluoromethyl)quinoline-4-carboxamide,
7-fluoro-N⁴-[5-methyl-1-(1,3-oxazol-2-ylmethyl)-3-(trifluoromethyl)-1H-pyrazol-4-yl]quinoline-2,4-dicarboxamide,
N-(3,5-dimethyl-1-{[2-(morpholin-4-yl)-1,3-thiazol-4-yl]methyl}-1H-pyrazol-4-yl)-2-methoxyquinoline-4-carboxamide,
N⁴-{1-[(3-ethyl-1,2-oxazol-5-yl)methyl]-3,5-dimethyl-1H-pyrazol-4-yl}quinoline-2,4-dicarboxamide,
N⁴-{1-[(3-ethyl-1,2-oxazol-5-yl)methyl]-3,5-dimethyl-1H-pyrazol-4-yl}-7-fluoroquinoline-2,4-dicarboxamide,
6-bromo-N-(1-{[1-(ethylsulfonyl)piperidin-4-yl]methyl}-3,5-dimethyl-1H-pyrazol-4-yl)-2-(trifluoromethyl)quinoline-4-carboxamide,
N⁴-(1-{[1-(ethylsulfonyl)piperidin-4-yl]methyl}-3,5-dimethyl-1H-pyrazol-4-yl)-7-fluoroquinoline-2,4-dicarboxamide,
N⁴-{1-[(5-ethyl-1,2,4-oxadiazol-3-yl)methyl]-5-methyl-3-(trifluoromethyl)-1H-pyrazol-4-yl}quinoline-2,4-dicarboxamide,
N⁴-{1-[(3-ethyl-1,2,4-oxadiazol-5-yl)methyl]-5-methyl-3-(trifluoromethyl)-1H-pyrazol-4-yl}quinoline-2,4-dicarboxamide,
N⁴-{1-[(3-ethyl-1,2,4-oxadiazol-5-yl)methyl]-5-methyl-3-(trifluoromethyl)-1H-pyrazol-4-yl}-7-fluoroquinoline-2,4-dicarboxamide,
N⁴-{5-methyl-1-[(5-methyl-1,2-oxazol-3-yl)methyl]-3-(trifluoromethyl)-1H-pyrazol-4-yl}quinoline-2,4-dicarboxamide,
7-fluoro-N⁴-{5-methyl-1-[(5-methyl-1,2-oxazol-3-yl)methyl]-3-(trifluoromethyl)-1H-pyrazol-4-yl}quinoline-2,4-dicarboxamide,
N⁴-[5-methyl-1-(1,3-oxazol-2-ylmethyl)-3-(trifluoromethyl)-1H-pyrazol-4-yl]quinoline-2,4-dicarboxamide,
N⁴-{3,5-dimethyl-1-[(5-methyl-1,2-oxazol-3-yl)methyl]-1H-pyrazol-4-yl}-7-fluoroquinoline-2,4-dicarboxamide,
7-fluoro-N⁴-{5-methyl-1-[(3-methyl-1,2-oxazol-5-yl)methyl]-3-(trifluoromethyl)-1H-pyrazol-4-yl}quinoline-2,4-dicarboxamide,
N⁴-{5-methyl-1-[(3-methyl-1,2-oxazol-5-yl)methyl]-3-(trifluoromethyl)-1H-pyrazol-4-yl}quinoline-2,4-dicarboxamide,
6-chloro-N⁴-{1-[(3-ethyl-1,2,4-oxadiazol-5-yl)methyl]-5-methyl-3-(trifluoromethyl)-1H-pyrazol-4-yl}-7-fluoroquinoline-2,4-dicarboxamide,
N⁴-[1-{[1-(ethylsulfonyl)piperidin-4-yl]methyl}-5-methyl-3-(trifluoromethyl)-1H-pyrazol-4-yl]-7-fluoroquinoline-2,4-dicarboxamide,
N⁴-[1-{[1-(ethylsulfonyl)piperidin-4-yl]methyl}-5-methyl-3-(trifluoromethyl)-1H-pyrazol-4-yl]quinoline-2,4-dicarboxamide,
6-chloro-7-fluoro-N⁴-[5-methyl-1-(1,3-oxazol-2-ylmethyl)-3-(trifluoromethyl)-1H-pyrazol-4-yl]quinoline-2,4-dicarboxamide,
N⁴-{1-[(5-ethyl-1,2,4-oxadiazol-3-yl)methyl]-5-methyl-3-(trifluoromethyl)-1H-pyrazol-4-yl}-7-fluoroquinoline-2,4-dicarboxamide,
N⁴-{1-[(5-cyclopropyl-1,2-oxazol-3-yl)methyl]-5-methyl-3-(trifluoromethyl)-1H-pyrazol-4-yl}quinoline-2,4-dicarboxamide,
N⁴-{1-[(5-cyclopropyl-1,2-oxazol-3-yl)methyl]-5-methyl-3-(trifluoromethyl)-1H-pyrazol-4-yl}-7-fluoroquinoline-2,4-dicarboxamide,
{1-[(5-cyclopropyl-1,2-oxazol-3-yl)methyl]-5-methyl-3-(trifluoromethyl)-1H-pyrazol-4-yl}-7-fluoroquinoline-2,4-dicarboxamide,
N⁴-{1-[(3-isopropyl-1,2-oxazol-5-yl)methyl]-5-methyl-3-(trifluoromethyl)-1H-pyrazol-4-yl}quinoline-2,4-dicarboxamide,
7-fluoro-N⁴-{1-[(3-isopropyl-1,2-oxazol-5-yl)methyl]-5-methyl-3-(trifluoromethyl)-1H-pyrazol-4-yl}quinoline-2,4-dicarboxamide,
6-chloro-7-fluoro-N⁴-{1-[(3-isopropyl-1,2-oxazol-5-yl)methyl]-5-methyl-3-(trifluoromethyl)-1H-pyrazol-4-yl}quinoline-2,4-dicarboxamide,
N⁴-{1-[(3-ethyl-1,2-oxazol-5-yl)methyl]-5-methyl-3-(trifluoromethyl)-1H-pyrazol-4-yl}quinoline-2,4-dicarboxamide,
N⁴-{1-[(3-ethyl-1,2-oxazol-5-yl)methyl]-5-methyl-3-(trifluoromethyl)-1H-pyrazol-4-yl}-7-fluoroquinoline-2,4-dicarboxamide,
N⁴-[5-methyl-1-{[5-(methylcarbamoyl)-1,2,4-oxadiazol-3-yl]methyl}-3-(trifluoromethyl)-1H-pyrazol-4-yl]quinoline-2,4-dicarboxamide,
6-chloro-7-fluoro-N⁴-[5-methyl-1-{[5-(methylcarbamoyl)-1,2,4-oxadiazol-3-yl]methyl}-3-(trifluoromethyl)-1H-pyrazol-4-yl]quinoline-2,4-dicarboxamide,
7-fluoro-N⁴-[5-methyl-1-{[5-(methylcarbamoyl)-1,2,4-oxadiazol-3-yl]methyl}-3-(trifluoromethyl)-1H-pyrazol-4-yl]quinoline-2,4-dicarboxamide,
N⁴-{3,5-dimethyl-1-[(3-methyl-1,2-oxazol-5-yl)methyl]-1H-pyrazol-4-yl}quinoline-2,4-dicarboxamide,
N⁴-{3,5-dimethyl-1-[(3-methyl-1,2-oxazol-5-yl)methyl]-1H-pyrazol-4-yl}-7-fluoroquinoline-2,4-dicarboxamide,
N⁴-{1-[(3-isopropyl-1,2-oxazol-5-yl)methyl]-3,5-dimethyl-1H-pyrazol-4-yl}quinoline-2,4-dicarboxamide,
7-fluoro-N⁴-{1-[(3-isopropyl-1,2-oxazol-5-yl)methyl]-3,5-dimethyl-1H-pyrazol-4-yl}quinoline-2,4-dicarboxamide,
N-[1-(4-cyanobenzyl)-5-methyl-3-(trifluoromethyl)-1H-pyrazol-4-yl]-6-fluoro-2-(morpholin-4-ylcarbonyl)quinoline-4-carboxamide,
N⁴-[1-(4-cyanobenzyl)-5-methyl-3-(trifluoromethyl)-1H-pyrazol-4-yl]-5-hydroxyquinoline-2,4-dicarboxamide,
N⁴-{1-[(3-cyano-1,2-oxazol-5-yl)methyl]-3,5-dimethyl-1H-pyrazol-4-yl}-7-fluoroquinoline-2,4-dicarboxamide,
N⁴-{1-[(1,5-dimethyl-1H-pyrazol-4-yl)methyl]-3,5-dimethyl-1H-pyrazol-4-yl}quinoline-2,4-dicarboxamide,
N⁴-{3,5-dimethyl-1-[(2-methyl-1,3-thiazol-4-yl)methyl]-1H-pyrazol-4-yl}quinoline-2,4-dicarboxamide,
N⁴-{3,5-dimethyl-1-[(1-methyl-1H-pyrazol-3-yl)methyl]-1H-pyrazol-4-yl}quinoline-2,4-dicarboxamide,
6-bromo-N-{1-[(3-ethyl-1,2,4-oxadiazol-5-yl)methyl]-3,5-dimethyl-1H-pyrazol-4-yl}-2-(trifluoromethyl)quinoline-4-carboxamide,
N⁴-{1-[(3-ethyl-1,2,4-oxadiazol-5-yl)methyl]-3,5-dimethyl-1H-pyrazol-4-yl}quinoline-2,4-dicarboxamide,
N-[1-(4-cyanobenzyl)-5-methyl-3-(trifluoromethyl)-1H-pyrazol-4-yl]-2-sulfamoylquinoline-4-carboxamide,
N-[1-(4-cyanobenzyl)-3,5-dimethyl-1H-pyrazol-4-yl]-2-sulfamoylquinoline-4-carboxamide,
N-[1-(4-cyanobenzyl)-5-methyl-3-(trifluoromethyl)-1H-pyrazol-4-yl]-2-(methylsulfamoyl)quinoline-4-carboxamide,
N-[1-(4-cyanobenzyl)-3,5-dimethyl-1H-pyrazol-4-yl]-2-(methylsulfamoyl)quinoline-4-carboxamide,
N-[1-(4-cyanobenzyl)-5-methyl-3-(trifluoromethyl)-1H-pyrazol-4-yl]-2-(dimethylsulfamoyl)quinoline-4-carboxamide,
N-[1-(4-cyanobenzyl)-3,5-dimethyl-1H-pyrazol-4-yl]-2-(dimethylsulfamoyl)quinoline-4-carboxamide,
N⁴-[1-(3-cyanobenzyl)-5-methyl-3-(trifluoromethyl)-1H-pyrazol-4-yl]-7-fluoroquinoline-2,4-dicarboxamide,
N⁴-[1-(3-cyanobenzyl)-5-methyl-3-(trifluoromethyl)-1H-pyrazol-4-yl]quinoline-2,4-dicarboxamide,
6-chloro-N⁴-[1-(3-cyanobenzyl)-5-methyl-3-(trifluoromethyl)-1H-pyrazol-4-yl]-7-fluoroquinoline-2,4-dicarboxamide,
N⁴-[1-(3-cyanobenzyl)-5-methyl-3-(trifluoromethyl)-1H-pyrazol-4-yl]-5-fluoroquinoline-2,4-dicarboxamide,
7-fluoro-N⁴-{1-[4-(methoxymethyl) benzyl]-3,5-dimethyl-1H-pyrazol-4-yl}quinoline-2,4-dicarboxamide,
6-chloro-N⁴-{3,5-dimethyl-1-[3-(trifluoromethoxy)benzyl]-1H-pyrazol-4-yl}-7-fluoroquinoline-2,4-dicarboxamide,
5-fluoro-N⁴-{5-methyl-1-[3-(trifluoromethoxy)benzyl]-3-(trifluoromethyl)-1H-pyrazol-4-yl}quinoline-2,4-dicarboxamide,
N⁴-{3,5-dimethyl-1-[3-(trifluoromethoxy)benzyl]-1H-pyrazol-4-yl}quinoline-2,4-dicarboxamide,
N⁴-{3,5-dimethyl-1-[3-(trifluoromethoxy)benzyl]-1H-pyrazol-4-yl}-7-fluoroquinoline-2,4-dicarboxamide,
N⁴-{3,5-dimethyl-1-[3-(trifluoromethoxy)benzyl]-1H-pyrazol-4-yl}-5-fluoroquinoline-2,4-dicarboxamide,
7-fluoro-N⁴-[1-(2-methoxybenzyl)-5-methyl-3-(trifluoromethyl)-1H-pyrazol-4-yl]quinoline-2,4-dicarboxamide,
6-chloro-7-fluoro-N⁴-{5-methyl-3-(trifluoromethyl)-1-[4-(trifluoromethyl)benzyl]-1H-pyrazol-4-yl}quinoline-2,4-dicarboxamide,
7-fluoro-N⁴-{5-methyl-3-(trifluoromethyl)-1-[4-(trifluoromethyl) benzyl]-1H-pyrazol-4-yl}quinoline-2,4-dicarboxamide,
5-fluoro-N⁴-{5-methyl-3-(trifluoromethyl)-1-[4-(trifluoromethyl) benzyl]-1H-pyrazol-4-yl}quinoline-2,4-dicarboxamide,
N⁴-{1-[4-(cyanomethyl)benzyl]-5-methyl-3-(trifluoromethyl)-1H-pyrazol-4-yl}quinoline-2,4-dicarboxamide,
N⁴-{1-[4-(cyanomethyl)benzyl]-5-methyl-3-(trifluoromethyl)-1H-pyrazol-4-yl}-5-fluoroquinoline-2,4-dicarboxamide,
6-chloro-N⁴-{1-[4-(cyanomethyl)benzyl]-5-methyl-3-(trifluoromethyl)-1H-pyrazol-4-yl}-7-fluoroquinoline-2,4-dicarboxamide,
6-bromo-N⁴-{1-[4-(cyanomethyl) benzyl]-5-methyl-3-(trifluoromethyl)-1H-pyrazol-4-yl}quinoline-2,4-dicarboxamide,
N⁴-{[4-(2-cyanopropan-2-yl)benzyl]-5-methyl-3-(trifluoromethyl)-1H-pyrazol-4-yl}-7-fluoroquinoline-2,4-dicarboxamide,
N⁴-{[4-(2-cyanopropan-2-yl)benzyl]-5-methyl-3-(trifluoromethyl)-1H-pyrazol-4-yl}-5-fluoroquinoline-2,4-dicarboxamide,
6-chloro-N⁴-{3,5-dimethyl-1-[4-(trifluoromethyl) benzyl]-1H-pyrazol-4-yl}-7-fluoroquinoline-2,4-dicarboxamide,
N⁴-{3,5-dimethyl-1-[4-(trifluoromethyl) benzyl]-1H-pyrazol-4-yl}-7-fluoroquinoline-2,4-dicarboxamide,
N⁴-{3,5-dimethyl-1-[4-(trifluoromethyl) benzyl]-1H-pyrazol-4-yl}-5-fluoroquinoline-2,4-dicarboxamide,
N⁴-[1-(3-carbamoylbenzyl)-5-methyl-3-(trifluoromethyl)-1H-pyrazol-4-yl]-7-fluoroquinoline-2,4-dicarboxamide,
N⁴-[1-(3-carbamoylbenzyl)-5-methyl-3-(trifluoromethyl)-1H-pyrazol-4-yl]-5-fluoroquinoline-2,4-dicarboxamide,
N⁴-[1-(3-carbamoylbenzyl)-5-methyl-3-(trifluoromethyl)-1H-pyrazol-4-yl]-6-chloro-7-fluoroquinoline-2,4-dicarboxamide,
N⁴-[1-(4-acetylbenzyl)-5-methyl-3-(trifluoromethyl)-1H-pyrazol-4-yl]quinoline-2,4-dicarboxamide,
N⁴-[1-(4-acetylbenzyl)-5-methyl-3-(trifluoromethyl)-1H-pyrazol-4-yl]-7-fluoroquinoline-2,4-dicarboxamide,
N⁴-[1-(4-acetylbenzyl)-5-methyl-3-(trifluoromethyl)-1H-pyrazol-4-yl]-5-fluoroquinoline-2,4-dicarboxamide,
N⁴-[1-(4-acetylbenzyl)-3,5-dimethyl-1H-pyrazol-4-yl]quinoline-2,4-dicarboxamide,
N⁴-[1-(4-acetylbenzyl)-3,5-dimethyl-1H-pyrazol-4-yl]-7-fluoroquinoline-2,4-dicarboxamide,
N⁴-[1-(4-acetylbenzyl)-3,5-dimethyl-1H-pyrazol-4-yl]-5-fluoroquinoline-2,4-dicarboxamide,
N⁴-[1-(4-cyanobenzyl)-5-methyl-3-(trifluoromethyl)-1H-pyrazol-4-yl]-7-methylquinoline-2,4-dicarboxamide,
6-bromo-N⁴-[1-(4-cyanobenzyl)-5-methyl-3-(trifluoromethyl)-1H-pyrazol-4-yl]-7-fluoroquinoline-2,4-dicarboxamide,
N⁴-{1-[4-(cyanomethyl)benzyl]-3,5-dimethyl-1H-pyrazol-4-yl}-5-fluoroquinoline-2,4-dicarboxamide,
N⁴-{1-[4-(cyanomethyl)benzyl]-3,5-dimethyl-1H-pyrazol-4-yl}-7-fluoroquinoline-2,4-dicarboxamide,
N⁴-{1-[4-(cyanomethyl)benzyl]-3,5-dimethyl-1H-pyrazol-4-yl}quinoline-2,4-dicarboxamide,
(±)-N⁴-{1-[4-(1-hydroxyethyl)benzyl]-5-methyl-3-(trifluoromethyl)-1H-pyrazol-4-yl}quinoline-2,4-dicarboxamide,
N⁴-[1-(4-cyclobutylbenzyl)-5-methyl-3-(trifluoromethyl)-1H-pyrazol-4-yl]-7-fluoroquinoline-2,4-dicarboxamide,
N⁴-[1-(4-cyclobutylbenzyl)-5-methyl-3-(trifluoromethyl)-1H-pyrazol-4-yl]quinoline-2,4-dicarboxamide,
N⁴-[1-(4-cyclobutylbenzyl)-5-methyl-3-(trifluoromethyl)-1H-pyrazol-4-yl]-5-fluoroquinoline-2,4-dicarboxamide,
N⁴-[1-(3-acetylbenzyl)-5-methyl-3-(trifluoromethyl)-1H-pyrazol-4-yl]-7-fluoroquinoline-2,4-dicarboxamide,
N⁴-[1-(3-acetylbenzyl)-5-methyl-3-(trifluoromethyl)-1H-pyrazol-4-yl]-5-fluoroquinoline-2,4-dicarboxamide,
N⁴-[1-(3-acetylbenzyl)-5-methyl-3-(trifluoromethyl)-1H-pyrazol-4-yl]-6-chloro-7-fluoroquinoline-2,4-dicarboxamide,
N⁴-[1-(3-acetylbenzyl)-5-methyl-3-(trifluoromethyl)-1H-pyrazol-4-yl]quinoline-2,4-dicarboxamide,
N⁴-[1-(3-acetylbenzyl)-3,5-dimethyl-1H-pyrazol-4-yl]-6-chloro-7-fluoroquinoline-2,4-dicarboxamide,
N⁴-[1-(3-acetylbenzyl)-3,5-dimethyl-1H-pyrazol-4-yl]-5-fluoroquinoline-2,4-dicarboxamide,
N⁴-[1-(3-acetylbenzyl)-3,5-dimethyl-1H-pyrazol-4-yl]-7-fluoroquinoline-2,4-dicarboxamide,
N⁴-[1-(3-acetylbenzyl)-3,5-dimethyl-1H-pyrazol-4-yl]quinoline-2,4-dicarboxamide,
N⁴-[1-(2,6-difluorobenzyl)-5-methyl-3-(trifluoromethyl)-1H-pyrazol-4-yl]-7-fluoroquinoline-2,4-dicarboxamide,
N⁴-[1-(2,6-difluorobenzyl)-5-methyl-3-(trifluoromethyl)-1H-pyrazol-4-yl]quinoline-2,4-dicarboxamide,
N⁴-[1-(2,6-difluorobenzyl)-5-methyl-3-(trifluoromethyl)-1H-pyrazol-4-yl]-5-fluoroquinoline-2,4-dicarboxamide,
6-chloro-N⁴-[1-(2,6-difluorobenzyl)-5-methyl-3-(trifluoromethyl)-1H-pyrazol-4-yl]-7-fluoroquinoline-2,4-dicarboxamide,
N⁴-[1-(2,6-difluorobenzyl)-3,5-dimethyl-1H-pyrazol-4-yl]-7-fluoroquinoline-2,4-dicarboxamide,
N⁴-[1-(2,6-difluorobenzyl)-3,5-dimethyl-1H-pyrazol-4-yl]quinoline-2,4-dicarboxamide,
N⁴-[1-(2,6-difluorobenzyl)-3,5-dimethyl-1H-pyrazol-4-yl]-5-fluoroquinoline-2,4-dicarboxamide,
6-chloro-N⁴-[1-(2,6-difluorobenzyl)-3,5-dimethyl-1H-pyrazol-4-yl]-7-fluoroquinoline-2,4-dicarboxamide,
(±)-N⁴-{1-[4-(1-hydroxyethyl)benzyl]-3,5-dimethyl-1H-pyrazol-4-yl}quinoline-2,4-dicarboxamide,
N⁴-{5-methyl-1-[3-(methylsulfonyl)benzyl]-3-(trifluoromethyl)-1H-pyrazol-4-yl}quinoline-2,4-dicarboxamide,
7-fluoro-N⁴-{5-methyl-1-[3-(methylsulfonyl)benzyl]-3-(trifluoromethyl)-1H-pyrazol-4-yl}quinoline-2,4-dicarboxamide,
6-chloro-7-fluoro-N⁴-{5-methyl-1-[3-(methylsulfonyl)benzyl]-3-(trifluoromethyl)-1H-pyrazol-4-yl}quinoline-2,4-dicarboxamide,
5-fluoro-N⁴-{5-methyl-1-[3-(methylsulfonyl)benzyl]-3-(trifluoromethyl)-1H-pyrazol-4-yl}quinoline-2,4-dicarboxamide,
N⁴-{3,5-dimethyl-1-[3-(methylsulfonyl)benzyl]-1H-pyrazol-4-yl}-7-fluoroquinoline-2,4-dicarboxamide,
N⁴-{3,5-dimethyl-1-[3-(methylsulfonyl)benzyl]-1H-pyrazol-4-yl}-5-fluoroquinoline-2,4-dicarboxamide,
N⁴-{3,5-dimethyl-1-[3-(methylsulfonyl)benzyl]-1H-pyrazol-4-yl}quinoline-2,4-dicarboxamide,
6-chloro-N⁴-{3,5-dimethyl-1-[3-(methylsulfonyl)benzyl]-1H-pyrazol-4-yl}-7-fluoroquinoline-2,4-dicarboxamide,
(±)-N⁴-{1-[3-(1-hydroxyethyl)benzyl]-5-methyl-3-(trifluoromethyl)-1H-pyrazol-4-yl}quinoline-2,4-dicarboxamide,
(±)-N⁴-{1-[3-(1-hydroxyethyl)benzyl]-3,5-dimethyl-1H-pyrazol-4-yl}quinoline-2,4-dicarboxamide,
N⁴-[1-(4-cyano-2-methoxybenzyl)-5-methyl-3-(trifluoromethyl)-1H-pyrazol-4-yl]-7-fluoroquinoline-2,4-dicarboxamide,
N⁴-[1-(4-cyano-2-methoxybenzyl)-5-methyl-3-(trifluoromethyl)-1H-pyrazol-4-yl]-5-fluoroquinoline-2,4-dicarboxamide,
6-chloro-N⁴-[1-(4-cyano-2-methoxybenzyl)-5-methyl-3-(trifluoromethyl)-1H-pyrazol-4-yl]-7-fluoroquinoline-2,4-dicarboxamide,
N⁴-[1-(4-cyano-2-methoxybenzyl)-3,5-dimethyl-1H-pyrazol-4-yl]-7-fluoroquinoline-2,4-dicarboxamide,
N⁴-[1-(4-cyano-2-methoxybenzyl)-3,5-dimethyl-1H-pyrazol-4-yl]-5-fluoroquinoline-2,4-dicarboxamide,
6-chloro-N⁴-[1-(4-cyano-2-methoxybenzyl)-3,5-dimethyl-1H-pyrazol-4-yl]-7-fluoroquinoline-2,4-dicarboxamide,
N⁴-[1-(2-cyano-5-fluorobenzyl)-5-methyl-3-(trifluoromethyl)-1H-pyrazol-4-yl]quinoline-2,4-dicarboxamide,
N⁴-[1-(2-cyano-5-fluorobenzyl)-5-methyl-3-(trifluoromethyl)-1H-pyrazol-4-yl]-7-fluoroquinoline-2,4-dicarboxamide,
N⁴-[1-(2-cyano-5-fluorobenzyl)-5-methyl-3-(trifluoromethyl)-1H-pyrazol-4-yl]-5-fluoroquinoline-2,4-dicarboxamide,
6-chloro-N⁴-[1-(2-cyano-5-fluorobenzyl)-5-methyl-3-(trifluoromethyl)-1H-pyrazol-4-yl]-7-fluoroquinoline-2,4-dicarboxamide,
N⁴-[1-(2-cyano-4-fluorobenzyl)-5-methyl-3-(trifluoromethyl)-1H-pyrazol-4-yl]-5-fluoroquinoline-2,4-dicarboxamide,
6-chloro-N⁴-[1-(2-cyano-4-fluorobenzyl)-5-methyl-3-(trifluoromethyl)-1H-pyrazol-4-yl]-7-fluoroquinoline-2,4-dicarboxamide,
N⁴-[1-(2-cyano-4-fluorobenzyl)-5-methyl-3-(trifluoromethyl)-1H-pyrazol-4-yl]-7-fluoroquinoline-2,4-dicarboxamide,
N⁴-[1-(2-cyano-4-fluorobenzyl)-5-methyl-3-(trifluoromethyl)-1H-pyrazol-4-yl]quinoline-2,4-dicarboxamide,
N⁴-[1-(2-cyano-4-fluorobenzyl)-3,5-dimethyl-1H H-pyrazol-4-yl]-7-fluoroquinoline-2,4-dicarboxamide,
N⁴-[1-(2-cyano-4-fluorobenzyl)-3,5-dimethyl-1H-pyrazol-4-yl]quinoline-2,4-dicarboxamide,
N⁴-[1-(2-cyano-4-fluorobenzyl)-3,5-dimethyl-1H-pyrazol-4-yl]-5-fluoroquinoline-2,4-dicarboxamide,
6-chloro-N⁴-[1-(2-cyano-4-fluorobenzyl)-3,5-dimethyl-1H-pyrazol-4-yl]-7-fluoroquinoline-2,4-dicarboxamide,
N⁴-[1-(4-cyano-3-methoxybenzyl)-5-methyl-3-(trifluoromethyl)-1H-pyrazol-4-yl]-7-fluoroquinoline-2,4-dicarboxamide,
N⁴-[1-(4-cyano-3-methoxybenzyl)-5-methyl-3-(trifluoromethyl)-1H-pyrazol-4-yl]-5-fluoroquinoline-2,4-dicarboxamide,
6-chloro-N⁴-[1-(4-cyano-3-methoxybenzyl)-5-methyl-3-(trifluoromethyl)-1H-pyrazol-4-yl]-7-fluoroquinoline-2,4-dicarboxamide,
N⁴-{1-[4-(2-cyanopropan-2-yl)benzyl]-5-methyl-3-(trifluoromethyl)-1H-pyrazol-4-yl}quinoline-2,4-dicarboxamide,
N⁴-[1-(4-cyano-2-hydroxybenzyl)-5-methyl-3-(trifluoromethyl)-1H-pyrazol-4-yl]-7-fluoroquinoline-2,4-dicarboxamide,
N⁴-{1-[(3-cyano-1,2-oxazol-5-yl)methyl]-5-methyl-3-(trifluoromethyl)-1H-pyrazol-4-yl}-7-fluoroquinoline-2,4-dicarboxamide,
ethyl 5-[(4-{[(2-carbamoyl-7-fluoroquinolin-4-yl)carbonyl]amino}-3,5-dimethyl-1H-pyrazol-1-yl)methyl]-1,2-oxazole-3-carboxylate,
N⁴-[1-{2-[(2-hydroxyphenyl)amino]-2-oxoethyl}-5-methyl-3-(trifluoromethyl)-1H-pyrazol-4-yl]quinoline-2,4-dicarboxamide,
N⁴-{1-[(3-cyano-1,2-oxazol-5-yl)methyl]-5-methyl-3-(trifluoromethyl)-1H-pyrazol-4-yl}quinoline-2,4-dicarboxamide,
6-chloro-N⁴-{1-[(3-cyano-1,2-oxazol-5-yl)methyl]-5-methyl-3-(trifluoromethyl)-1H-pyrazol-4-yl}-7-fluoroquinoline-2,4-dicarboxamide,
N⁴-{1-[(2-cyano-1,3-thiazol-4-yl)methyl]-5-methyl-3-(trifluoromethyl)-1H-pyrazol-4-yl}-7-fluoroquinoline-2,4-dicarboxamide,
N⁴-{1-[(5-cyano-2-thienyl)methyl]-5-methyl-3-(trifluoromethyl)-1H-pyrazol-4-yl}-7-fluoroquinoline-2,4-dicarboxamide,
6-chloro-7-fluoro-N⁴-[5-methyl-1-{[5-(morpholin-4-ylmethyl)-1,2-oxazol-3-yl]methyl}-3-(trifluoromethyl)-1H-pyrazol-4-yl]quinoline-2,4-dicarboxamide,
ethyl 5-{[4-{[(2-carbamoyl-7-fluoroquinolin-4-yl)carbonyl]amino}-5-methyl-3-(trifluoromethyl)-1H-pyrazol-1-yl]methyl}-1,2-oxazole-3-carboxylate,
N⁴-[1-(1,3-benzoxazol-2-ylmethyl)-5-methyl-3-(trifluoromethyl)-1H-pyrazol-4-yl]-7-fluoroquinoline-2,4-dicarboxamide,
N⁴-{1-[(5-cyano-2-thienyl)methyl]-5-methyl-3-(trifluoromethyl)-1H-pyrazol-4-yl}quinoline-2,4-dicarboxamide,
methyl 2-{[4-{[(2-carbamoylquinolin-4-yl)carbonyl]amino}-5-methyl-3-(trifluoromethyl)-1H-pyrazol-1-yl]methyl}-1,3-oxazole-4-carboxylate,
methyl 2-{[4-{[(2-carbamoyl-7-fluoroquinolin-4-yl)carbonyl]amino}-5-methyl-3-(trifluoromethyl)-1H-pyrazol-1-yl]methyl}-1,3-oxazole-4-carboxylate,
methyl 2-{[4-{[(2-carbamoyl-6-chloro-7-fluoroquinolin-4-yl)carbonyl]amino}-5-methyl-3-(trifluoromethyl)-1H-pyrazol-1-yl]methyl}-1,3-oxazole-4-carboxylate,
ethyl 5-{[4-{[(2-carbamoyl-6-chloro-7-fluoroquinolin-4-yl)carbonyl]amino}-5-methyl-3-(trifluoromethyl)-1H-pyrazol-1-yl]methyl}-1,2-oxazole-3-carboxylate,
2-{[4-{[(2-carbamoylquinolin-4-yl)carbonyl]amino}-5-methyl-3-(trifluoromethyl)-1H-pyrazol-1-yl]methyl}-1,3-oxazole-4-carboxylic acid,
2-{[4-{[(2-carbamoyl-7-fluoroquinolin-4-yl)carbonyl]amino}-5-methyl-3-(trifluoromethyl)-1H-pyrazol-1-yl]methyl}-1,3-oxazole-4-carboxylic acid,
2-{[4-{[(2-carbamoyl-6-chloro-7-fluoroquinolin-4-yl)carbonyl]amino}-5-methyl-3-(trifluoromethyl)-1H-pyrazol-1-yl]methyl}-1,3-oxazole-4-carboxylic acid,
N⁴-{1-[(6-cyanopyridin-2-yl)methyl]-5-methyl-3-(trifluoromethyl)-1H-pyrazol-4-yl}-7-fluoroquinoline-2,4-dicarboxamide,
N⁴-{1-[(6-cyanopyridin-2-yl)methyl]-5-methyl-3-(trifluoromethyl)-1H-pyrazol-4-yl}-5-fluoroquinoline-2,4-dicarboxamide,
6-chloro-N⁴-{1-[(6-cyanopyridin-2-yl)methyl]-5-methyl-3-(trifluoromethyl)-1H-pyrazol-4-yl}-7-fluoroquinoline-2,4-dicarboxamide,
N⁴-{1-[(6-cyanopyridin-2-yl)methyl]-5-methyl-3-(trifluoromethyl)-1H-pyrazol-4-yl}quinoline-2,4-dicarboxamide,
N⁴-(1-{2-[(3-cyanophenyl)amino]-2-oxoethyl}-3,5-dimethyl-1H-pyrazol-4-yl)quinoline-2,4-dicarboxamide,
N⁴-(1-{2-[(4-fluorophenyl)amino]-2-oxoethyl}-3,5-dimethyl-1H-pyrazol-4-yl)quinoline-2,4-dicarboxamide,
N⁴-(1-{2-[(4-fluorophenyl)(methyl)amino]-2-oxoethyl}-3,5-dimethyl-1H-pyrazol-4-yl)quinoline-2,4-dicarboxamide,
N⁴-[1-(4-cyano-3-methylbenzyl)-5-methyl-3-(trifluoromethyl)-1H-pyrazol-4-yl]-5-fluoroquinoline-2,4-dicarboxamide,
N⁴-[1-(4-cyano-3-methylbenzyl)-5-methyl-3-(trifluoromethyl)-1H-pyrazol-4-yl]-7-fluoroquinoline-2,4-dicarboxamide,
6-chloro-N⁴-[1-(4-cyano-3-methylbenzyl)-3,5-dimethyl-1H-pyrazol-4-yl]-7-fluoroquinoline-2,4-dicarboxamide,
N⁴-[1-(4-cyano-3-methylbenzyl)-3,5-dimethyl-1H-pyrazol-4-yl]-5-fluoroquinoline-2,4-dicarboxamide,
N⁴-[1-(4-cyano-3-methylbenzyl)-3,5-dimethyl-1H-pyrazol-4-yl]-7-fluoroquinoline-2,4-dicarboxamide,
6-chloro-N⁴-[1-(2-cyano-5-methylbenzyl)-3,5-dimethyl-1H-pyrazol-4-yl]-7-fluoroquinoline-2,4-dicarboxamide,
N⁴-[1-(2-cyano-5-methylbenzyl)-5-methyl-3-(trifluoromethyl)-1H-pyrazol-4-yl]-7-fluoroquinoline-2,4-dicarboxamide,
N⁴-(1-{2-[(3-fluorophenyl)amino]-2-oxoethyl}-3,5-dimethyl-1H-pyrazol-4-yl)quinoline-2,4-dicarboxamide,
N⁴-[5-methyl-1-{[5-(morpholin-4-ylmethyl)-1,2-oxazol-3-yl]methyl}-3-(trifluoromethyl)-1H-pyrazol-4-yl]quinoline-2,4-dicarboxamide,
7-fluoro-N⁴-{5-methyl-1-[2-(trifluoromethoxy)benzyl]-3-(trifluoromethyl)-1H-pyrazol-4-yl}quinoline-2,4-dicarboxamide,
6-chloro-7-fluoro-N⁴-{5-methyl-1-[2-(trifluoromethoxy)benzyl]-3-(trifluoromethyl)-1H-pyrazol-4-yl}quinoline-2,4-dicarboxamide,
N⁴-{5-methyl-1-[2-(trifluoromethoxy)benzyl]-3-(trifluoromethyl)-1H-pyrazol-4-yl}quinoline-2,4-dicarboxamide,
5-fluoro-N⁴-{5-methyl-1-[2-(trifluoromethoxy)benzyl]-3-(trifluoromethyl)-1H-pyrazol-4-yl}quinoline-2,4-dicarboxamide,
N⁴-{1-[(6-cyanopyridin-2-yl)methyl]-3,5-dimethyl-1H-pyrazol-4-yl}-7-fluoroquinoline-2,4-dicarboxamide,
N⁴-{1-[(6-cyanopyridin-2-yl)methyl]-3,5-dimethyl-1H-pyrazol-4-yl}quinoline-2,4-dicarboxamide,
N⁴-{1[(6-cyanopyridin-2-yl)methyl]-3,5-dimethyl-1H-pyrazol-4-yl}-5-fluoroquinoline-2,4-dicarboxamide,
7-fluoro-N⁴-[1-(4-fluoro-2-methylbenzyl)-5-methyl-3-(trifluoromethyl)-1H-pyrazol-4-yl]quinoline-2,4-dicarboxamide,
5-fluoro-N⁴-[1-(4-fluoro-2-methylbenzyl)-5-methyl-3-(trifluoromethyl)-1H-pyrazol-4-yl]quinoline-2,4-dicarboxamide,
6-chloro-7-fluoro-N⁴-[1-(4-fluoro-2-methylbenzyl)-5-methyl-3-(trifluoromethyl)-1H-pyrazol-4-yl]quinoline-2,4-dicarboxamide,
N⁴-[1-(4-fluoro-2-methylbenzyl)-5-methyl-3-(trifluoromethyl)-1H-pyrazol-4-yl]quinoline-2,4-dicarboxamide,
7-fluoro-N⁴-{5-methyl-1-[4-(2,2,2-trifluoroethyl)benzyl]-3-(trifluoromethyl)-1H-pyrazol-4-yl}quinoline-2,4-dicarboxamide,
5-fluoro-N⁴-{5-methyl-1-[4-(2,2,2-trifluoroethyl)benzyl]-3-(trifluoromethyl)-1H-pyrazol-4-yl}quinoline-2,4-dicarboxamide,
6-chloro-7-fluoro-N⁴-{5-methyl-1-[4-(2,2,2-trifluoroethyl)benzyl]-3-(trifluoromethyl)-1H-pyrazol-4-yl}quinoline-2,4-dicarboxamide,
N⁴-{5-methyl-1-[4-(2,2,2-trifluoroethyl)benzyl]-3-(trifluoromethyl)-1H-pyrazol-4-yl}quinoline-2,4-dicarboxamide,
or a tautomer, an N-oxide, a hydrate, a solvate, or a salt thereof, or a mixture of same.

16. A method of preparing a compound according to claim 1, comprising reacting an intermediate of formula (II) 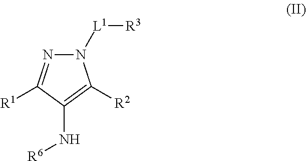
in which R¹, R², R³, R⁶ and L¹ are as defined in claim 1;
with a compound of formula (III) 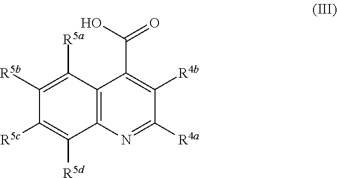
in which R^{4a}, R^{4b}, R^{5a}, R^{5b}, R^{5c}, and R^{5d} are as defined in claim 1;
thus providing a compound of formula (I) 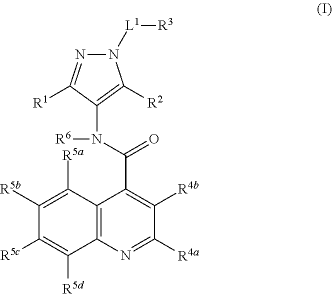
in which R¹, R², R³, R^{4a}, R^{4b}, R^{5a}, R^{5b}, R^{5b}, R^{5d}, R⁶, and L¹ are as defined in claim 1.

17. A compound of formula (II) 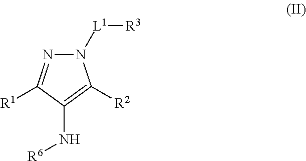
in which R¹, R², R³, R⁶ and L¹ are as defined in claim 1.

18. A compound of formula (III) 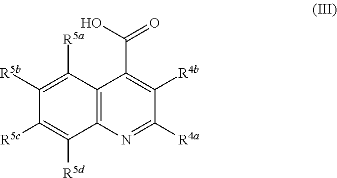
in which R^{4a}, R^{4b}, R^{5a}, R^{5b}, R^{5c}, and R^{5d} are as defined in claim 1.

19. (canceled)

20. (canceled)

21. A pharmaceutical composition comprising a compound of formula (I) according to claim 1, or a tautomer, an N-oxide, a hydrate, a solvate, or a pharmaceutically acceptable salt thereof, or a mixture of same, and a pharmaceutically acceptable diluent or carrier.

22. A pharmaceutical combination comprising:
one or more compounds of formula (I) according to claim 1, or a tautomer, an N-oxide, a hydrate, a solvate, or a pharmaceutically acceptable salt thereof, or a mixture of same;
and
one or more agents selected from: a taxane, Docetaxel, Paclitaxel, or Taxol; an epothilone, Ixabepilone, Patupilone, or Sagopilone; Mitoxantrone; Predinisolone; Dexamethasone; Estramustin; Vinblastin; Vincristin; Doxorubicin; Adriamycin; Idarubicin; Daunorubicin; Bleomycin; Etoposide; Cyclophosphamide; Ifosfamide; Procarbazine; Melphalan; 5-Fluorouracil; Capecitabine; Fludarabine; Cytarabine; Ara-C; 2-Chloro-2-deoxyadenosine; Thioguanine; an anti-androgen, Flutamide, Cyproterone acetate, or Bicalutamide; Bortezomib; a platinum derivative, Cisplatin, or Carboplatin; Chlorambucil; Methotrexate; and Rituximab.

23. (canceled)

24. (canceled)

25. A method for the treatment of a disease of uncontrolled cell growth, proliferation or survival, an inappropriate cellular immune response, or an inappropriate cellular inflammatory response mediated by GLUT1 comprising administering to a subject in need thereof a therapeutically effective amount of a compound of formula (I) according to claim 1, or a tautomer, an N-oxide, a hydrate, a solvate, or a pharmaceutically acceptable salt thereof, or a mixture of same.

26. The method according to claim 25, wherein the disease of uncontrolled cell growth, proliferation or survival, inappropriate cellular immune response, or inappropriate cellular inflammatory response is a haemotological tumour, solid tumour or metastases thereof.

27. The method according to claim 26, wherein the haemotological tumour, solid tumour or metastases thereof is selected from leukaemias and myelodysplastic syndrome, malignant lymphomas, head and neck tumours, brain tumours and brain metastases, tumours of the thorax, non-small cell and small cell lung tumours, gastrointestinal tumours, endocrine tumours, mammary and other gynaecological tumours, urological tumours, renal, bladder and prostate tumours, skin tumours, and sarcomas, and metastases thereof.
